(19) United States
(12) Patent Application Publication
(10) Pub. No.: US 2025/0179198 A1
(43) Pub. Date: Jun. 5, 2025

(54) COMPOSITIONS AND METHODS RELATED TO IL27 RECEPTOR BINDING

(71) Applicant: Synthekine, Inc., Menlo Park, CA (US)

(72) Inventors: Robert Kastelein, Menlo Park, CA (US); Patrick Lupardus, Menlo Park, CA (US); Deepti Rokkam, Menlo Park, CA (US)

(21) Appl. No.: 18/946,556

(22) Filed: Nov. 13, 2024

Related U.S. Application Data
(63) Continuation of application No. 18/464,998, filed on Sep. 11, 2023, now Pat. No. 12,209,132.
Application No. 18/464,998 is a continuation of application No. 18/164,386, filed on Feb. 3, 2023, now Pat. No. 11,873,349.
Application No. 18/164,386 is a continuation of application No. PCT/US2021/044577, filed on Aug. 4, 2021.
(60) Provisional application No. 63/078,745, filed on Sep. 15, 2020.
(60) Provisional application No. 63/135,884, filed on Jan. 11, 2021.

(51) Int. Cl. C07K 16/28; A61K 39/00; A61P 37/02; C07K 14/715; C07K 16/46; C07K 19/00; C12N 15/63; C12N 15/86

(52) U.S. Cl. CPC C07K 16/2866; A61P 37/02; C07K 14/7155; C07K 16/2803; C07K 16/46; C07K 16/468; C07K 19/00; C12N 15/63; C12N 15/86; A61K 2039/505; C07K 2317/22; C07K 2317/24; C07K 2317/31; C07K 2317/33; C07K 2317/522; C07K 2317/524; C07K 2317/526; C07K 2317/53; C07K 2317/565; C07K 2317/567; C07K 2317/569; C07K 2319/40

(57) ABSTRACT

Provided herein are IL27R binding proteins that bind to IL27Rα and gp130 and comprise an anti-IL27Rα V_{H}H antibody and an anti-gp130 V_{H}H antibody.

### CROSS-REFERENCES TO RELATED APPLICATIONS

This application is a continuation of U.S. application Ser. No. 18/464,998, filed Sep. 11, 2023, which is a continuation of U.S. application Ser. No. 18/164,386, filed Feb. 3, 2023 which is a continuation of PCT/US2021/044577, filed Aug. 4, 2021, which claims priority to U.S. Provisional Application No. 63/061,562, filed Aug. 5, 2020, U.S. Provisional Application No. 63/078,745, filed Sep. 15, 2020, and U.S. Provisional Application No. 63/135,884, filed Jan. 11, 2021, the disclosures of which are hereby incorporated by reference in their entirety for all purposes.

### SEQUENCE LISTING

The instant application contains a Sequence Listing which has been submitted electronically in XML format and is hereby incorporated by reference in its entirety. Said XML file, created on Feb. 2, 2023, is named 106249-1361753-006820US_SL.xml, and is 2,426,545 bytes in size.

### BACKGROUND OF THE DISCLOSURE

The interleukin-27 receptor (IL27R) is a type I cytokine receptor for interleukin-27 (IL27). It is a heterodimer composed of the IL27Rα subunit and glycoprotein 130 (gp130). IL27 is expressed by antigen presenting cells and induces differentiation of a diverse populations of T cells in the immune system. When IL27 binds to the IL27R, signaling pathways, such as the JAK-STAT and p38 MAPK pathways, are turned on to induce pro-inflammatory or anti-inflammatory responses, which involve different types of cells, such as macrophages, dendritic cells, T cells, and B cells. The response that is activated can be dependent on the external surrounding of IL27.

IL-27 is a heterodimeric cytokine consisting of two non-covalently linked subunits, p28 and EBI3. The p28 subunit belongs to the 4-helix bundle cytokine family, while EBI3 is the shortest form possible of a soluble cytokine receptor, with two typical cytokine binding domains (Pflanz S, et al., Immunity. 2002 June; 16 (6): 779-90).

The primary binding receptor for IL-27 is the IL-27R1 (also known as TCCR- or WSX-1 receptor). IL-27 and IL-27R1 form a complex of considerable affinity (nM). Gp130 is the second receptor that binds the complex of IL-27/IL-27R to create the active signaling complex. Gp130 binding to the IL-27/IL-27R1 complex is much weaker than the interaction between IL-27 and IL-27R1 (Pflanz S, et al., J Immunol. 2004 Feb. 15; 172 (4): 2225-31).

The IL-27 extracellular domain has 5 domains. The first two domains form the IL-27 binding domains. Typically the loops between D1 and D2 provide most of the binding energy.

The other 3 domains are called Fibronectin type III domains (Fn3). The sequence of each Fn3 domain varies.

The gp130 receptor has 6 domains. The top domain D1 of gp130 binds p28 of IL-27. D2 and D3 contribute little to binding IL-27. The membrane proximal 3 domains are Fn3 domains. The sequence of each Fn3 domain varies.

The structure of the IL-27R is not known but the domain structure is known. The structure of gp130 is known as complexed with IL-6. Based on that structure it is evident that the Fn3 domains do not energetically contribute to IL-27R complex formation. Rather the structure of the gp130 forms a ‘C’ with the domain 4 and 5 at 80% angle of each other. Certain residues in each of the ‘tall’ receptors are conserved similar to D4 and D5 in gp130. This indicates that all tall receptors of the gp130 family, including IL-27R, forms this ‘C’ structure. (Yibin Xu, et al., J Biol Chem. 2010 Jul. 9: 285 (28): 21214-8).

IL-27R has 5 extracellular domains. D1 and D2 are the cytokine binding domains. D3, D4 and D5 are Fn3 domains. The D5 domain of IL-27R and the D6 domain of gp130 will come close together at the membrane because of the ‘C’ shape of each receptor. This is required to be able for the receptor complex to trigger binding of JAKs at the intracellular domains of both receptors.

### SUMMARY OF THE DISCLOSURE

The present disclosure provides compositions useful in the pairing of cellular receptors to generate desirable effects useful in treatment of disease in mammalian subjects.

Several advantages flow from the binding molecules described herein. The natural ligand of the IL-27R, IL-27, causes gp130 and IL-27Rα to come into proximity (i.e., by their simultaneous binding of IL-27). However, when IL-27 is used as a therapeutic in mammalian, particularly human, subjects, it may also trigger a number of adverse and undesirable effects by a variety of mechanisms including the presence of gp130 and IL-27Rα on other cell types and the binding to gp130 and IL-27Rα on the other cell types may result in undesirable effects and/or undesired signaling on cells expressing gp130 and IL-27Rα. The present disclosure is directed to methods and compositions that modulate the multiple effects of gp130 and IL-27Rα binding so that desired therapeutic signaling occurs, particularly in a desired cellular or tissue subtype, while minimizing undesired activity and/or intracellular signaling.

In some embodiments, the IL-27R binding molecules described herein are partial agonists of the IL-27 receptor. In some embodiments, the binding molecules described herein are designed such that the binding molecules are full agonists. In some embodiments, the binding molecules described herein are designed such that the binding molecules are super agonists.

In some embodiments, the binding molecules provide the maximal desired IL-27 intracellular signaling from binding to gp130 and IL-27Rα on the desired cell types, while providing significantly less IL-27 signaling on other undesired cell types. This can be achieved, for example, by selection of binding molecules having differing affinities or causing different Eₘₐₓ, for gp130 and IL-27Rα as compared to the affinity of IL-27 for gp130 and IL-27Rα. Because different cell types respond to the binding of ligands to its cognate receptor with different sensitivity, by modulating the affinity of the dimeric ligand (or its individual binding moieties) for the IL-27 receptor relative to wild-type IL-27 binding may facilitate the stimulation of desired activities while reducing undesired activities on non-target cells.

The present disclosure provides bivalent binding molecules that are agonists of the IL-27 receptor, the bivalent binding molecule comprising:
a first single domain antibody (sdAb) that specifically binds to the extracellular domain of gp130 (an “anti-gp130 sdAb”), and
a second single domain antibody that specifically binds to extracellular domain IL-27Rα (an “anti-IL-27Rα sdAb”),
   wherein the anti-gp130 sdAb and anti-IL-27Rα sdAb are stably associated and first wherein contacting a cell expressing gp130 and IL-27Rα with an effective amount of the bivalent binding molecule results the dimerization of gp130 and IL-27Rα and results in intracellular signaling characteristic of the IL-27 receptor when activated by its natural cognate IL-27. In some embodiments, one or both of the sdAbs is an scFv. In some embodiments, one or both of the sdAbs is a V_{H}H.

In some embodiments, one sdAb of the bivalent binding molecule is an scFv and the other sdAb is a V_{H}H.

In some embodiments, the first and second sdAbs are covalently bound via a chemical linkage.

In some some embodiments, the first and second sdAbs are provided as single continuous polypeptide.

In some embodiments, the first and second sdAbs are provided as single continuous polypeptide optionally comprising an intervening polypeptide linker between the amino acid sequences of the first and second sdAbs.

In some embodiments the bivalent binding molecule optionally comprising a linker, is optionally expressed as a fusion protein with an additional amino acid sequence. In some embodiments, the additional amino acid sequence is a purification handle such as a chelating peptide or an additional protein such as a subunit of an Fc molecule.

In one aspect, the disclosure provides an IL27 receptor (IL27R) binding protein that specifically binds to IL27Rα subunit (IL27Rα) and glycoprotein 130 subunit (gp130), wherein the binding protein causes the multimerization of IL27Rα and gp130 when bound to IL27Rα and gp130 and the multimerization results in the activation of JAK kinases associated with the intracellular domains of IL27Rα and gp130 and intracelluar signaling, and wherein the binding protein comprises a single-domain antibody (sdAb) that specifically binds to IL27Rα (an anti-IL27Rα sdAb) and a sdAb that specifically binds to gp130 (an anti-gp130 sdAb). In some embodiments, the multimerization of IL27Rα and gp130 can cause downstream signaling.

In some embodiments, the anti-IL27Rα sdAb is a V_{H}H antibody (an anti IL27Rα V_{H}H antibody) and/or the anti-gp130 sdAb is a V_{H}H antibody (an anti gp130 V_{H}H antibody). In some embodiments, the anti-IL27Rα sdAb and the anti-gp130 sdAb are joined directly or via a peptide linker. In some embodiments, the peptide linker comprises between 1 and 50 amino acids. In particular embodiments, the peptide linker comprises a sequence of GGGS (SEQ ID NO: 108).

In some embodiments, the IL27R binding protein comprises:
a first V_{H}H antibody comprising a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR1 from a row of Table 1A; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR2 from the same row of Table 1A; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR3 from the same row of Table 1A; and
a second V_{H}H antibody comprising a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR4 from the same row of Table 1A; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR5 from the same row of Table 1A; and a CDR3 having at least 90% (e.g., 91%, 92%, 93%, 94%, 95%, 96%, 97%, 98%, 99%, or 100%) sequence identity, or having 0, 1, 2, or 3 amino acid changes optionally conservative amino acid changes relative, to the sequence of a CDR6 from the same row of Table 1A.

In some embodiments, the IL27R binding protein comprises a sequence at least 90% (e.g., 91%, 92%, 93%, 94%, 95%, 96%, 97%, 98%, 99%, or 100%) identical to any one of the dual V_{H}H dimer sequences shown in Table 1A.

In certain embodiments, the anti IL27Rα V_{H}H antibody comprises a CDR1 having 0, 1, 2, or 3 amino acid changes relative to the sequence of any one of SEQ ID NOS: 193-198; a CDR2 having 0, 1, 2, or 3 amino acid changes relative to the sequence of any one of SEQ ID NOS: 199-204; and a CDR3 having 0, 1, 2, or 3 amino acid changes relative to the sequence of any one of SEQ ID NOS: 205-210. In further embodiments, the anti gp130 V_{H}H antibody comprises a CDR1 having 0, 1, 2, or 3 amino acid changes relative to the sequence of any one of SEQ ID NOS: 211-217; a CDR2 having 0, 1, 2, or 3 amino acid changes relative to the sequence of any one of SEQ ID NOS: 218-224; and a CDR3 having 0, 1, 2, or 3 amino acid changes relative to the sequence of any one of SEQ ID NOS: 225-231.

In particular embodiments, the IL27R binding protein comprises a CDR1, a CDR2, and a CDR3 in the anti IL27Rα V_{H}H antibody and a CDR1, a CDR2, and a CDR3 in the anti gp130 V_{H}H antibody as listed in a row of Table 1.

In some embodiments, the binding protein comprises an anti gp130 V_{H}H antibody linked to the N-terminus of a linker and an anti IL27Rα V_{H}H antibody linked to the C-terminus of the linker. In some embodiments, the anti gp130 V_{H}H antibody comprises a sequence having at least 90% (e.g., 91%, 92%, 93%, 94%, 95%, 96%, 97%, 98%, 99%, or 100%) sequence identity to a sequence of any one of SEQ ID NOS: 232-237. In some embodiments, the anti IL27Rα V_{H}H antibody comprises a sequence having at least 90% (e.g., 91%, 92%, 93%, 94%, 95%, 96%, 97%, 98%, 99%, or 100%) sequence identity to a sequence of any one of SEQ ID NOS: 238-244.

In particular embodiments, each of the anti-gp130 V_{H}H antibody and the anti-IL27Rα V_{H}H antibody comprises a sequence having at least 90% (e.g., 91%, 92%, 93%, 94%, 95%, 96%, 97%, 98%, 99%, or 100%) identity to a sequence listed in a row of Table 2A.

In certain embodiments, the binding protein comprises a sequence that is substantially identical to a sequence of any one of SEQ ID NOS: 1-42. In certain embodiments, the binding protein comprises a sequence having at least 90% (e.g., 91%, 92%, 93%, 94%, 95%, 96%, 97%, 98%, 99%, or 100%) identity to a sequence of any one of SEQ ID NOS: 1-42.

In some embodiments, the binding protein comprises an anti IL27Rα V_{H}H antibody linked to the N-terminus of a linker and an anti gp130 V_{H}H antibody linked to the C-terminus of the linker. In some embodiments, the anti IL27Rα V_{H}H antibody comprises a sequence having at least 90% (e.g., 91%, 92%, 93%, 94%, 95%, 96%, 97%, 98%, 99%, or 100%) sequence identity to a sequence of any one of SEQ ID NOS: 245-251. In certain embodiments, the anti gp130 V_{H}H antibody comprises a sequence having at least 90% sequence identity to a sequence of any one of SEQ ID NOS: 252-257.

In particular embodiments, each of the anti IL27Rα V_{H}H antibody and the anti gp130 V_{H}H antibody comprises a sequence having at least 90% (e.g., 91%, 92%, 93%, 94%, 95%, 96%, 97%, 98%, 99%, or 100%) identity to a sequence listed in a row of Table 3A.

In certain embodiments, the binding protein comprises a sequence that is substantially identical to a sequence of any one of SEQ ID NOS: 43-84. In certain embodiments, the binding protein comprises a sequence having at least 90% (e.g., 91%, 92%, 93%, 94%, 95%, 96%, 97%, 98%, 99%, or 100%) identity to a sequence of any one of SEQ ID NOS: 43-84.

In another aspect, the disclosure provides an isolated nucleic acid encoding the IL27R binding protein described herein. In certain embodiments, the isolated nucleic acid comprises a sequence having at least 90% (e.g., 91%, 92%, 93%, 94%, 95%, 96%, 97%, 98%, 99%, or 100%) sequence identity to a sequence of any one of SEQ ID NOS: 109-192 or a sequence from Table 1B. The disclosure also provides an expression vector comprising the nucleic acid. The disclosure also provides an isolated host cell comprising the expression vector.

In another aspect, the disclosure provides a pharmaceutical composition comprising the IL27R binding protein described herein and a pharmaceutically acceptable carrier.

In another aspect, the disclosure provides a method of treating an autoimmune or inflammatory disease, disorder, or condition, a neoplastic disease, or a viral infection in a subject in need thereof, comprising administering to the subject a therapeutically effective amount of an IL27R binding protein described herein or a pharmaceutical composition described herein.

In some embodiments, the method further comprises administering one or more supplementary agents selected from the group consisting of a corticosteroid, a Janus kinase inhibitor, a calcineurin inhibitor, a mTor inhibitor, an IMDH inhibitor, a biologies, a vaccine, and a therapeutic antibody. In certain embodiments, the therapeutic antibody is an antibody that binds a protein selected from the group consisting of BLyS, CD11a, CD20, CD25, CD3, CD52, IgEIL12/IL23, IL17a, IL1ß, IL4Rα, IL5, IL6R, integrin-α4β7, RANKL, TNFα, VEGF-A, and VLA-4.

In certain embodiments, the disease, disorder, or condition is selected from viral infections, Helicobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathie arthritis, juvenile reactive arthritis, juvenile Reiter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathie arthritis, reactive arthritis, Reiter's syndrome, SEA Syndrome, psoriasis, psoriatic arthritis, dermatitis (eczema), exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid, urticaria, prokeratosis, rheumatoid arthritis; seborrheic dermatitis, solar dermatitis, seborrheic keratosis, senile keratosis, actinic keratosis, photo-induced keratosis, keratosis follicularis; acne vulgaris; keloids; nevi; warts including verruca, condyloma or condyloma acuminatum, and human papilloma viral (HPV) infections.

An IL27R binding proteins described herein are useful in the treatment of neoplastic diseases, such as cancer (e.g., a solid tumor cancer; e.g., non-small-cell lung carcinoma (NSCLC), renal cell carcinoma (RCC), or melanoma) and/or infectious diseases (e.g., bacterial infections and viral infections (e.g., viral infections caused by hepatitis C virus (HCV), human papillomavirus (HPV), or human immunodeficiency virus (HIV)) in a subject in need thereof. The IL27R binding protein binds to and activates CD8⁺ T cells, CD4⁺ T cells, and/or T regulatory (Treg) cells. The IL27R binding protein can trigger different levels of downstream signaling in different cell types. For example, by varying the length of the linker between the antiIL27Rα V_{H}H antibody and the antigp130 V_{H}H antibody in the IL27R binding protein, the IL27R binding protein can cause a higher level of downstream signaling in desired cell types compared to undesired cell types. In some embodiments, by varying the linker length, an IL27R binding protein can cause a higher level of downstream signaling in T cells (e.g., CD8⁺ T cells) compared to the level of downstream signaling in other cells. In other embodiments, different antiIL27Rα V_{H}H antibodies with different binding affinities and different antigp130 V_{H}H antibodies with different binding affinities can be combined to make different IL27R binding proteins. Further, the orientation of the two antibodies in the binding protein can also be changed to make a different binding protein (i.e., antiIL27Rα V_{H}H antibody-linker-antigp130 V_{H}H antibody, or antigp130 V_{H}H antibody-linker-antiIL27Rα V_{H}H antibody). Different IL27R binding proteins can be screened to find the ideal binding protein that causes a higher level of downstream signaling in desired cell types compared to undesired cell types. In some embodiments, the level of downstream signaling in T cells (e.g., CD8⁺ T cells) is at least 1.1, 1.5, 2, 3, 5, or 10 times of the level of downstream signaling in other cells.

In particular, the IL27R binding protein binds to and activates CD8⁺ T cells. In some embodiments, the IL27R binding protein binds to and activates CXCR5⁺ CD8⁺ T cells. It is known that IL27 can promote and sustain a rapid division of memory-like CXCR5⁺ CD8⁺ T cells during, for example, viral infection. The CXCR5⁺ CD8⁺ T cells can sustain T cell responses during persistent infection or cancer and drive the proliferative burst of CD8⁺ T cells after anti-PD1 treatment. Accordingly, an IL27R binding protein described herein is useful to sustain and augment self-renewing T cells in chronic infections and neoplastic diseases, such as cancer.

### BRIEF DESCRIPTION OF THE FIGURES

FIG. 1 of the attached drawings provides a schematic representation of one embodiment of the bivalent binding molecule of the present disclosure comprising a first single domain antibody (1) and a second single domain antibody (3) and a linker (2) depicted as interacting with a cell membrane (10) associated heterodimeric receptor comprising a first receptor subunit comprising an extracellular domain (4), and transmembrane domain (5) and an intracellular domain (6) interaction of a bivalent binding molecule and a second first receptor subunit comprising an extracellular domain (7), and transmembrane domain (8) and an intracellular domain (9) wherein the intracellular domain of the first receptor (6) and the intracellular domain of the second receptor (9) on of a bivalent binding molecule are within a proximal distance (11).

FIGS. 2A and 2B of the attached drawings provides a schematic representation of two illustrative configurations of bivalent binding molecules of the present disclosure. FIG. 2A provides a schematic representation of an illustrative single polypeptide chain bivalent binding molecule comprising, from amino to carboxy, a first single domain antibody (1) and a second single domain antibody (3) and a linker (2). FIG. 2B provides a schematic representation of a bivalent binding molecule comprising a first single domain antibody (1) and a second single domain antibody (3) and a linker (2) and a knob-into-hole Fc domain, the Fc domain comprising a first subunit which is a Fc knob (13) and a second subunit which is a Fc hole (14) wherein the bivalent binding molecule is covalently linked to an Fc domain subunit via a IgG hinge sequence (12).

FIGS. 3A and 3B of the attached drawings provides a schematic representations of two illustrative configurations of bivalent binding molecules of the present disclosure. FIG. 3A provides a schematic representation of an illustrative bivalent binding molecule construct comprising two bivalent binding molecules each attached to a subunit of a knob-into-hole Fc domain, the construct comprising two polypeptide chains, the first polypeptide chain comprising, from amino to carboxy, a first single domain antibody (1), a linker (2) and a second single domain antibody (3), a IgG hinge sequence (12) and a Fe knob subunit (13) and a second polypeptide chain comprising, from amino to carboxy, a first single domain antibody (1), a linker (2) and a second single domain antibody (3), a IgG hinge sequence (12) and a Fe hole subunit (14) wherein the first and second polypeptides are in stable associate via the interaction of the knob-into-hole Fc domain. FIG. 3B provides schematic representation of a an alternative arrangement of a bivalent binding molecule construct comprising two polypeptides a first polypeptide chain comprising, from amino to carboxy, a first single domain antibody (1), a linker (2) and a second single domain antibody (3), an IgG hinge sequence (12) and a Fe knob subunit (13) and a second polypeptide chain comprising, from amino to carboxy, a first second domain antibody (3), a linker (2) and a first single domain antibody (1), a IgG hinge sequence (12) and a Fc hole subunit (14), wherein the first and second polypeptides are in stable association via the interaction of the knob-into-hole Fc domain.

FIG. 4A provides alternative schematic representations of configurations of the bivalent binding molecules of the present disclosure where one single domain antibody is attached to each subunit of a knob-into-hole Fc domain comprising two polypeptides, the first polypeptide comprising from amino to carboxy, a first single domain antibody (1), an IgG hinge sequence (12) and a Fc knob subunit (13), the second polypeptide comprising from amino to carboxy, a second single domain antibody (3), an IgG binge sequence (12) and a Fe hole subunit (13), wherein the first and second single domain antibodies are in stable associate via the interaction of the knob-into-hole Fc domain.

FIG. 4B provides a schematic representation of a binding molecule the binding domains are single domain antibodies associated via transition metal coordinate covalent complex. As illustrated, the binding molecules comprises two polypeptide subunits: the first subunit comprising a first single domain antibody (1) is attached via a first linker (15) to a first chelating peptide (17) and second subunit comprising a second single domain antibody (3) is attached via a second linker (16) to a second chelating peptide (18), wherein the first chelating peptide (17) and second chelating peptide (18) form a coordinate covalent complex with a single transition metal ion (“M”). The transition metal ion may be in a kinetically labile or kinetically inert oxidation state.

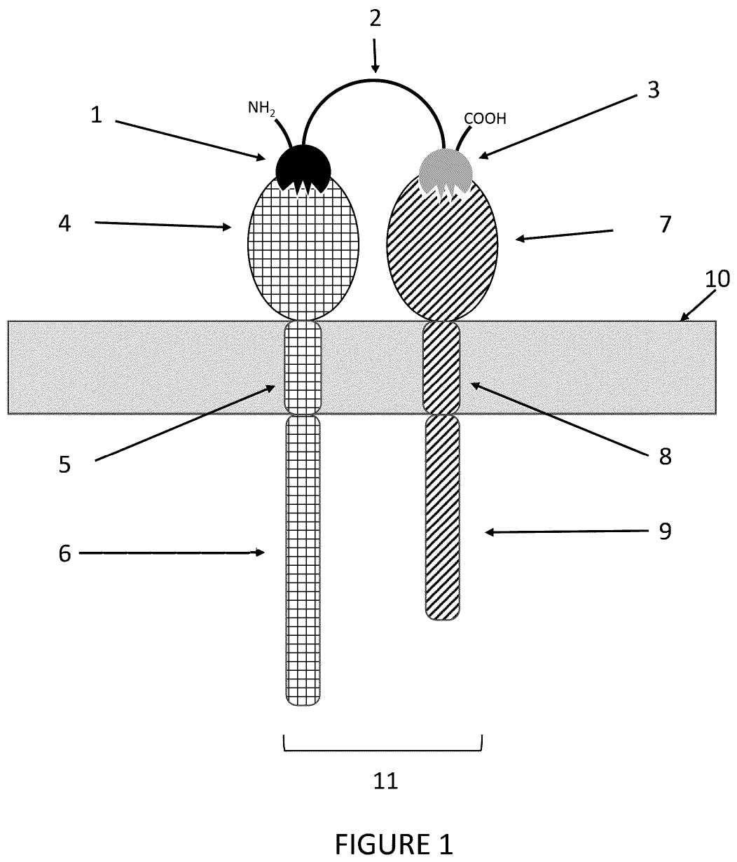

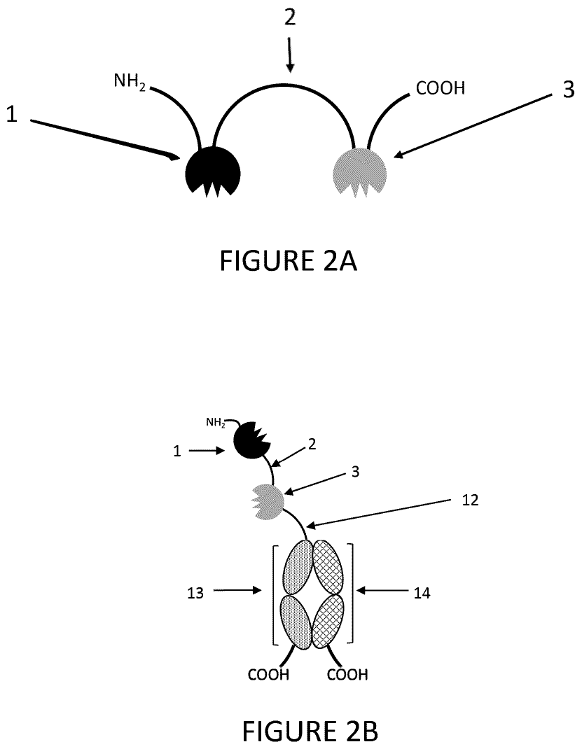

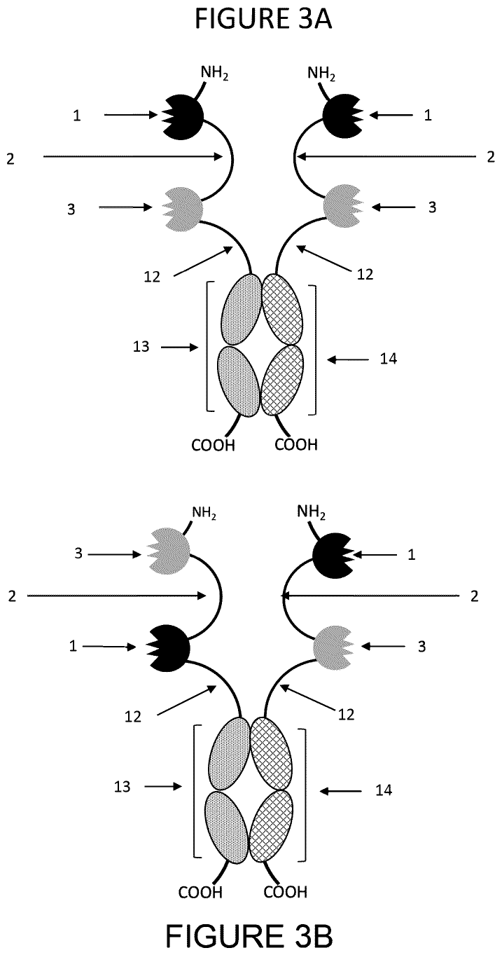

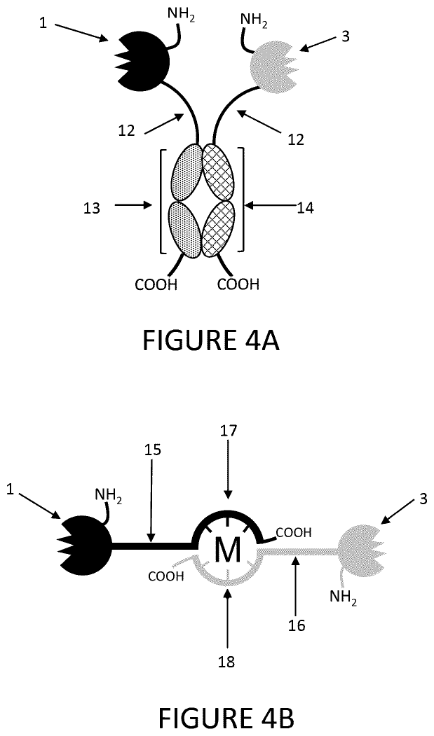

### DETAILED DESCRIPTION OF THE DISCLOSURE

### I. Introduction

The present disclosure provides compositions useful in the pairing of cellular receptors to generate desirable effects useful in treatment of diseases. In general, binding proteins are provided that comprise a first domain that binds to IL27Rα and a second domain that binds to gp130, such that upon contacting with a cell expressing IL27Rα and gp130, the binding protein causes the functional association of IL27Rα and gp130, thereby resulting in functional dimerization of the receptors and downstream signaling.

Several advantages flow from the binding proteins described herein. The natural ligand of IL27R, IL27, causes IL27Rα and gp130 to come into proximity (i.e., by their simultaneous binding of IL27). However, when IL27 is used as a therapeutic in mammalian, particularly human, subjects, it may also trigger a number of adverse and undesirable effects by a variety of mechanisms including the presence of IL27Rα and gp130 on other cell types and the binding to IL27Rα and gp130 on the other cell types may result in undesirable effects and/or undesired signaling on cells expressing IL27Rα and gp130. The present disclosure is directed to methods and compositions that modulate the multiple effects of IL27Rα and gp130 binding so that desired therapeutic signaling occurs, particularly in a desired cellular or tissue subtype, while minimizing undesired activity and/or intracellular signaling.

In some embodiments, the binding proteins described herein are designed such that the binding proteins provide the maximal desired IL27 intracellular signaling from binding to IL27Rα and gp130 on the desired cell types, while providing significantly less IL27 signaling on other undesired cell types. This can be achieved, for example, by selection of binding proteins having differing affinities or causing different Eₘₐₓ for IL27Rα and gp130 as compared to the affinity of IL27 for IL27Rα and gp130. Because different cell types respond to the binding of ligands to its cognate receptor with different sensitivity, by modulating the affinity of the dimeric ligand (or its individual binding moieties) for the IL27 receptor relative to wild-type IL27 binding facilitates the stimulation of desired activities while reducing undesired activities on non-target cells. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be affected by the level of downstream signaling caused by the binding protein, can also be measured.

### Interleukin 27 (IL27) Structure:

IL27 is a member of the IL-12 cytokine family. IL27 is a heterodimeric cytokine comprised of two subunits: IL27A (also referred to as IL-27p28) and IL27B (also referred to as Epstein-Barr Virus induced gene 3 or “EBI3”). The human p28 (hIL27A) is expressed as a 243 amino acid pre-protein comprising 28 amino acid signal sequence which is post-translationally removed to render a 215 amino acid mature protein. UniProtKB-QSNEV9 (IL27A_HUMAN). The mature form of p28 (less the signal peptide) possesses the amino acid sequence;

(SEQ ID NO: 400)
FPRPPGRPOLSLOELRREFTVSLHLARKLLSEVRGOAHRFAESHLPGVN
LYLLPLGEQLPDVSLTFQAWRRLSDPERLCFISTTLQPFHALLGGLGTQ
GRWTNMERMQLWAMRLDLRDLQRHLRFQVLAAGFNLPEEEEEEEEE
EEEERKGLLPGALGSALQGPAQVSWPQLLSTYRLLHSLELVLSRAVRE
LLLLSKAGHSVWPLGFPTLSPQP

The human IL27B (hIL27B) is expressed as a 229 amino acid pre-protein comprising 20 amino acid signal sequence which is post-translationally removed to render a 209 amino acid mature protein. UniProtKB-Q14213 (IL27B_HUMAN). The mature form of hIL27B (less the signal peptide) possesses the amino acid sequence

(SEQ ID NO: 401)
RKGPPAALTLPRVQCRASRYPIAVDCSWTLPPAPNSTSPVSFIATYRLG
MAARGHSWPCLQQTPTSTSCTITDVQLFSMAPYVLNVTAVHPWGSSS
SFVPFITEHIIKPDPPEGVRLSPLAERQLQVQWEPPGSWPFPEIFSLKYW
IRYKRQGAARFHRVGPIEATSFILRAVRPRARYYVQVAAQDLTDYGEL
SDWSLPATATMSLGK

### Interleukin 27 (IL27) Receptor:

IL27 results in intracellular signaling via its interaction with a heterodimeric receptor consisting of: IL-27Rα (or IL27RA) and gp130. The binding of IL27 to IL27 receptor activates signaling pathways including the JAK/STAT and p38 MAPK pathways. IL27 stimulates both pro-inflammatory and anti-inflammatory response in different cell types such as macrophages, dendritic cells, T cells and B cells. The type of response is dependent on the environment.

The human IL27 receptor subunit alpha (hIL27RA) is expressed as a 636 amino acid pre-protein comprising 32 amino acid signal sequence which is post-translationally removed to render a 604 amino acid mature protein. UniProtKB-Q6UWB1 (127RA_HUMAN)

The mature form of hIL27RA (less the signal peptide) possesses the amino acid sequence:

(SEQ ID NO: 402)
QGSAGPLQCYGVGPLGDLNCSWEPLGDLGAPSELHLQSQKYRSNKTQ
TVAVAAGRSWVAIPREQLTMSDKLLVWGTKAGQPLWPPVFVNLETQ
MKPNAPRLGPDVDFSEDDPLEATVHWAPPTWPSHKVLICQFHYRRCQ
EAAWTLLEPELKTIPLTPVEIQDLELATGYKVYGRCRMEKEEDLWGE
WSPILSFQTPPSAPKDVWVSGNLCGTPGGEEPLLLWKAPGPCVQVSYK
VWFWVGGRELSPEGITCCCSLIPSGAEWARVSAVNATSWEPLTNLSLV
CLDSASAPRSVAVSSIAGSTELLVTWQPGPGEPLEHVVDWARDGDPLE
KLNWVRLPPGNLSALLPGNFTVGVPYRITVTAVSASGLASASSVWGFR
EELAPLVGPTLWRLQDAPPGTPAIAWGEVPRHQLRGHLTHYTLCAQS
GTSPSVCMNVSGNTQSVTLPDLPWGPCELWVTASTIAGQGPPGPILRL
HLPDNTLRWKVLPGILFLWGLFLLGCGLSLATSGRCYHLRHKVLPRW
VWEKVPDPANSSSGQPHMEQVPEAQPLGDLPILEVEEMEPPPVMESSQ
PAQATAPLDSGYEKHELPTPEELGLLGPPRPQVLA

The extracellular domain of hIL27RA (IL27RA-ECD) is a 484 amino acid polypeptide corresponding to amino acids 33-516 of the hIL27RA preprotein and possesses the amino acid sequence:

(SEQ ID NO: 403)
QGSAGPLQCYGVGPLGDLNCSWEPLGDLGAPSELHLQSQKYRSNKTQ
TVAVAAGRSWVAIPREQLTMSDKLLVWGTKAGQPLWPPVFVNLETQ
MKPNAPRLGPDVDFSEDDPLEATVHWAPPTWPSHKVLICQFHYRRCQ
EAAWTLLEPELKTIPLTPVEIQDLELATGYKVYGRCRMEKEEDLWGE
WSPILSFQTPPSAPKDVWVSGNLCGTPGGEEPLLLWKAPGPCVQVSYK
VWFWVGGRELSPEGITCCCSLIPSGAEWARVSAVNATSWEPLTNLSLV
CLDSASAPRSVAVSSIAGSTELLVTWQPGPGEPLEHVVDWARDGDPLE
KLNWVRLPPGNLSALLPGNFTVGVPYRITVTAVSASGLASASSVWGFR
EELAPLVGPTLWRLQDAPPGTPAIAWGEVPRHQLRGHLTHYTLCAQS
GTSPSVCMNVSGNTQSVTLPDLPWGPCELWVTASTIAGQGPPGPILRL
HLPDNTLRWK

The human gp130 receptor subunit (hGP130) is also referred as the IL6 receptor beta subunit. UniProtKB-P40189 (IL6RB_HUMAN. hGP130 is expressed as a 918 amino acid pre-protein comprising 22 amino acid signal sequence which is post-translationally removed to render a 896 amino acid mature protein. The mature form of hGP130 possess the amino acid sequence:

(SEQ ID NO: 404)
ELLDPCGYISPESPVVQLHSNFTAVCVLKEKCMDYFHVNANYIVWKT
NHFTIPKEQYTIINRTASSVTFTDIASLNIQLTCNILTFGQLEQNVYGIT
SGLPPEKPKNLSCIVNEGKKMRCEWDGGRETHLETNFTLKSEWATHK
IIFADCKAKRDTPTSCTVDYSTVYFVNIEVWVEAENALGKVTSDHINED
PVYKVKPNPPHNLSVINSEELSSILKLTWTNPSIKSVIILKYNIQYRTKD
ASTWSQIPPEDTASTRSSFTVQDLKPFTEYVFRIRCMKEDGKGYWSDW
SEEASGITYEDRPSKAPSFWYKIDPSHTQGYRTVQLVWKTLPPFEANG
KILDYEVTLTRWKSHLQNYTVNATKLTVNLINDRYLATLTVRNLVGK
SDAAVLTIPACDFQATHPVMDLKAFPKDNMLWVEWTTPRESVKKYIL
EWCVLSDKAPCITDWQQEDGTVHRTYLRGNLAESKCYLITVTPVYAD
GPGSPESIKAYLKQAPPSKGPTVRTKKVGKNEAVLEWDQLPVDVQNG
FIRNYTIFYRTHIGNETAVNVDSSHTEYTLSSLTSDTLYMVRMAAYTDE
GGKDGPEFTFTTPKFAQGEIEAIVVPVCLAFLLTTLLGVLFCFNKRDLI
KKHIWPNVPDPSKSHIAQWSPHTPPRHNFNSKDQMYSDGNFTDVSVV
EIEANDKKPFPEDLKSLDLFKKEKINTEGHSSGIGGSSCMSSSRPSISSSD
ENESSQNTSSTVQYSTVVHSGYRHQVPSVQVFSRSESTQPLLDSEERPE
DLQLVDHVDGGDGILPRQQYFKQNCSQHESSPDISHFERSKQVSSVNE
EDFVRLKQQISDHISQSCGSGQMKMFQEVSAADAFGPGTEGQVERFET
VGMEAATDEGMPKSYLPQTVRQGGYMPQ

The extracellular domain of hGP130 (hGP130-ECD) is a 597 amino acid polypeptide corresponding to amino acids 23-619 of the hGP130 preprotein and possesses the amino acid sequence:

(SEQ ID NO: 405)
ELLDPCGYISPESPVVQLHSNFTAVCVLKEKCMDYFHVNANYIVWKT
NHFTIPKEQYTIINRTASSVTFTDIASLNIQLTCNILTFGQLEQNVYGIT
IISGLPPEKPKNLSCIVNEGKKMRCEWDGGRETHLETNFTLKSEWATHK
FADCKAKRDTPTSCTVDYSTVYFVNIEVWVEAENALGKVTSDHINFD
PVYKVKPNPPHNLSVINSEELSSILKLTWINPSIKSVIILKYNIQYRTKD
ASTWSQIPPEDTASTRSSFTVQDLKPFTEYVFRIRCMKEDGKGYWSDW
SEEASGITYEDRPSKAPSFWYKIDPSHTQGYRTVQLVWKTLPPFEANG
KILDYEVTLTRWKSHLQNYTVNATKLTVNLINDRYLATLTVRNLVGK
SDAAVLTIPACDFQATHPVMDLKAFPKDNMLWVEWTTPRESVKKYIL
EWCVLSDKAPCITDWQQEDGTVHRTYLRGNLAESKCYLITVTPVYAD
GPGSPESIKAYLKQAPPSKGPTVRTKKVGKNEAVLEWDQLPVDVQNG
FIRNYTIFYRTIIGNETAVNVDSSHTEYTLSSLTSDTLYMVRMAAYTDE
GGKDGPEFTFTTPKFAQGEIE

### IL27 Activity

IL27 is expressed by antigen presenting cells. hIL27 induces differentiation of the diverse populations of T cells in the immune system and also upregulates IL10. hIL27 has pro- and anti-inflammatory properties, that can regulate T-helper cell development, suppress T-cell proliferation, stimulate cytotoxic T-cell activity, induce isotype switching in B-cells, and that have diverse effects on innate immune cells. Among its target cells are CD4 T-helper cells which can differentiate in type 1 effector cells (TH1), type 2 effector cells (TH2) and IL17 producing helper T-cells (TH17).

### T Cell Differentiation

IL27 plays a significant role in the differentiation through inducing or suppressing of T cell subtypes including Th1, Th2, Th17, Tr1 and Treg cells. IL-27 is greatly involved in differentiation through inducing or suppressing of each T cell subset. Interferon gamma (IFNg) expressing Th1 cells are generated in response IL27 through STAT1 signaling via expression of T-bet and signature Thì genes. IL4 expressing Th2 cells are inhibited by IL27 through the transcription factor GATA-3. Th17 cells, which express IL17, IL22, and GM-CSF, are inhibited by IL27 through STAT1 and expression of transcription factor RORγt. Treg cells are inhibited by IL27 through STAT1 and STAT3.

IL27 drives rapid clonal expansion of naive but not memory CD4 T-cells. IL27 also strongly synergizes with IL-12 to trigger interferon-gamma/IFN-gamma production of naive CD4 T-cells, binds to the cytokine receptor WSX-1/TCCR. Another important role of IL-27 is its antitumor activity as well as its antiangiogenic activity with activation of production of antiangiogenic chemokines

### Induction of IL10

Tr1 cells which express IL-10, are induced by IL-27 through the transcription factor c-Maf providing an anti-inflammatory response. A primary activity of IL-10 is the suppression of inflammatory responses. Also involved are the STAT1 and STAT3 transcription factors that bind specifically to the IL-27a. The activation of STAT3 by IL-27 leads to an increase of IL-10 secretion from Treg cells.

### II. Definitions

To facilitate the understanding of present disclosure, certain terms and phrases are defined below as well as throughout the specification. The definitions provided herein are non-limiting and should be read in view of the knowledge of one of skill in the art would know.

Before the present methods and compositions are described, it is to be understood that this invention is not limited to a particular method or composition described, as such may, of course, vary. It is also to be understood that the terminology used herein is for the purpose of describing embodiments only and is not intended to be limiting.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It should be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless indicated otherwise, parts are parts by weight, molecular weight is weight average molecular weight, temperature is in degrees Celsius (° C.), and pressure is at or near atmospheric. Standard abbreviations are used, including the following: bp=base pair(s); kb=kilobase(s); pl=picoliter(s); s or sec=second(s); min=minute(s); h or hr=hour(s); AA or aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; l or L=liter; μM=micromolar; mM=millimolar; M=molar; kDa=kilodalton; i.m.=intramuscular(ly); i.p.=intraperitoneal(ly); SC or SQ=subcutaneous (ly); QD=daily; BID=twice daily; QW=once weekly; QM=once monthly; HPLC=high performance liquid chromatography; BW=body weight, U=unit; ns=not statistically significant; PBS=phosphate-buffered saline; PCR=polymerase chain reaction; HSA=human serum albumin; MSA=mouse serum albumin; DMEM=Dulbeco's Modification of Eagle's Medium: EDTA=ethylenediaminetetraacetic acid.

It will be appreciated that throughout this disclosure reference is made to amino acids according to the single letter or three letter codes. For the reader's convenience, the single and three letter amino acid codes are provided in the Table below:

TABLE
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Standard methods in molecular biology are described in the scientific literature (see, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1). cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., NY).

Activate: As used herein the term “activate” is used in reference to a receptor or receptor complex to reflect a biological effect, directly and/or by participation in a multicomponent signaling cascade, arising from the binding of an agonist ligand to a receptor responsive to the binding of the ligand.

Activity: As used herein, the term “activity” is used with respect to a molecule to describe a property of the molecule with respect to a test system (e.g. an assay) or biological or chemical property (e.g. the degree of binding of the molecule to another molecule) or of a physical property of a material or cell (e.g. modification of cell membrane potential). Examples of such biological functions include but are not limited to catalytic activity of a biological agent, the ability to stimulate intracellular signaling, gene expression, cell proliferation, the ability to modulate immunological activity such as inflammatory response. “Activity” is typically expressed as a level of a biological activity per unit of agent tested such as [catalytic activity]/[mg protein], [immunological activity]/[mg protein], international units (IU) of activity, [STAT5 phosphorylation]/[mg protein], [T-cell proliferation]/[mg protein], plaque forming units (pfu), etc. As used herein, the term “proliferative activity” referees to an activity that promotes cell proliferation and replication.

Administer/Administration: The terms “administration” and “administer” are used interchangeably herein to refer the act of contacting a subject, including contacting a cell, tissue, organ, or biological fluid of the subject in vitro, in vivo or ex vivo with an agent (e.g. an ortholog, an IL2 ortholog, an engineered cell expressing an orthogonal receptor, an engineered cell expressing an orthogonal IL2 receptor, a CAR-T cell expressing an orthogonal IL2 receptor, a chemotherapeutic agent, an antibody, or a pharmaceutical formulation comprising one or more of the foregoing). Administration of an agent may be achieved through any of a variety of art recognized methods including but not limited to the topical administration, intravascular injection (including intravenous or intraarterial infusion), intradermal injection, subcutaneous injection, intramuscular injection, intraperitoneal injection, inhalation and the like. The term “administration” includes contact of an agent to the cell, tissue or organ as well as the contact of an agent to a fluid, where the fluid is in contact with the cell, tissue or organ.

Affinity: As used herein the term “affinity” refers to the degree of specific binding of a first molecule (e.g., a ligand) to a second molecule (e.g., a receptor) and is measured by the equilibrium dissociation constant (KD), a ratio of the dissociation rate constant between the molecule and its target (Koff) and the association rate constant between the molecule and its target (Kon).

Agonist: As used herein, the term “agonist” refers a first agent that specifically binds a second agent (“target”) and interacts with the target to cause or promote an increase in the activation of the target. In some instances, agonists are activators of receptor proteins that modulate cell activation, enhance activation, sensitize cells to activation by a second agent, or up-regulate the expression of one or more genes, proteins, ligands, receptors, biological pathways, that may result in cell proliferation or pathways that result in cell cycle arrest of cell death such as by apoptosis. In some embodiments, an agonist is an agent that binds to a receptor and alters the receptor state, resulting in a biological response. The response mimics the effect of the endogenous activator of the receptor. The term “agonist” includes partial agonists, full agonists and superagonists. An agonist may be described as a “full agonist” when such agonist which leads to a substantially full biological response (i.e., the response associated with the naturally occurring ligand/receptor binding interaction) induced by receptor under study, or a partial agonist. In contrast to agonists, antagonists may specifically bind to a receptor but do not result the signal cascade typically initiated by the receptor and may to modify the actions of an agonist at that receptor. Inverse agonists are agents that produce a pharmacological response that is opposite in direction to that of an agonist. A “superagonist” is a type of agonist that is capable of producing a maximal response greater than the endogenous agonist for the target receptor, and thus has an activity of more than 100% of the native ligand. A super agonist is typically a synthetic molecule that exhibits greater than 110%, alternatively greater than 120%, alternatively greater than 130%, alternatively greater than 140%, alternatively greater than 150%, alternatively greater than 160%, or alternatively greater than 170% of the response in an evaluable quantitative or qualitative parameter of the naturally occurring form of the molecule when evaluated at similar concentrations in a comparable assay.

Antagonist: As used herein, the term “antagonist” or “inhibitor” refers a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist. Inhibitors are molecules that decrease, block, prevent, delay activation, inactivate, desensitize, or down-regulate, e.g., a gene, protein, ligand, receptor, biological pathway, or cell.

Antibody: As used herein, the term “antibody” refers collectively to: (a) glycosylated and non-glycosylated immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, V_{H}, V_{L}, tetrabodies, triabodies, diabodies, dsFv, F(ab′)₃, scFv-Fc and (scFv), that competes with the immunoglobulin from which it was derived for binding to the target molecule. The term antibody is not restricted to immunoglobulins derived from any particular mammalian species and includes murine, human, equine, and camelids antibodies (e.g., human antibodies). The term “antibody” encompasses antibodies isolatable from natural sources or from animals following immunization with an antigen and as well as engineered antibodies including monoclonal antibodies, bispecific antibodies, trispecific, chimeric antibodies, humanized antibodies, human antibodies, CDR-grafted, veneered, or deimmunized (e.g., to remove T-cell epitopes) antibodies. The term “human antibody” includes antibodies obtained from human beings as well as antibodies obtained from transgenic mammals comprising human immunoglobulin genes such that, upon stimulation with an antigen the transgenic animal produces antibodies comprising amino acid sequences characteristic of antibodies produced by human beings. The term “antibody” should not be construed as limited to any particular means of synthesis and includes naturally occurring antibodies isolatable from natural sources and as well as engineered antibodies molecules that are prepared by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries.

Binding molecule: As used herein, the term “binding molecule” refers to a bivalent molecule that can bind to the extracellular domain of two cell surface receptors. In some embodiments, a binding molecule specifically binds to two different receptors (or domains or subunits thereof) such that the receptors (or domains or subunits) are maintained in proximity to each other such that the receptors (or domains or subunits), including domains thereof (e.g., intracellular domains) interact with each other and result in downstream signaling.

CDR: As used herein, the term “CDR” or “complementarity determining region” is intended to mean the non-contiguous antigen combining sites found within the variable region of both heavy and light chain immunoglobulin polypeptides. CDRs have been described by Kabat et al., J. Biol. Chem. 252:6609-6616 (1977); Kabat, et al., U.S. Dept. of Health and Human Services publication entitled “Sequences of proteins of immunological interest” (1991) (also referred to herein as “Kabat 1991” or “Kabat”); by Chothia, et al. (1987) J. Mol. Biol. 196:901-917 (also referred to herein as “Chothia”); and MacCallum, et al. (1996) J. Mol. Biol. 262:732-745, where the definitions include overlapping or subsets of amino acid residues when compared against each other. Nevertheless, application of either definition to refer to a CDR of an antibody or grafted antibodies or variants thereof is intended to be within the scope of the term as defined and used herein. The term “Chothia Numbering” as used herein is recognized in the arts and refers to a system of numbering amino acid residues based on the location of the structural loop regions (Chothia et al. 1986, Science 233:755-758; Chothia & Lesk 1987, JMB 196:901-917; Chothia et al. 1992, JMB 227:799-817). For purposes of the present disclosure, unless otherwise specifically identified, the positioning of CDRs2 and 3 in the variable region of an antibody follows Kabat numbering or simply, “Kabat.” The positioning of CDR1 in the variableregion of an antibody follows a hybrid of Kabat and Chothia numbering schemes.

Clonotype: A clonotype is defined as a collection of binding molecules that originate from the same B-cell progenitor cell. The term “clonotype” is used herein to refer to a collection of antigen binding molecules that belong to the same germline family, have the same CDR3 lengths, and have 70% or greater homology in CDR3 sequence

Comparable: As used herein, the term “comparable” is used to describe the degree of difference in two measurements of an evaluable quantitative or qualitative parameter. For example, where a first measurement of an evaluable quantitative parameter and a second measurement of the evaluable parameter do not deviate beyond a range that the skilled artisan would recognize as not producing a statistically significant difference in effect between the two results in the circumstances, the two measurements would be considered “comparable.” In some instances, measurements may be considered “comparable” if one measurement deviates from another by less than 30%, alternatively by less than 25%, alternatively by less than 20%, alternatively by less than 15%, alternatively by less than 10%, alternatively by less than 7%, alternatively by less than 5%, alternatively by less than 4%, alternatively by less than 3%, alternatively by less than 2%, or by less than 1%. In particular embodiments, one measurement is comparable to a reference standard if it deviates by less than 15%, alternatively by less than 10%, or alternatively by less than 5% from the reference standard.

As used herein, the term “downstream signaling” refers to the cellular signaling process that is caused by the interaction of two or more cell surface receptors that are brought into proximity of each other.

Effective Concentration (EC): As used herein, the terms “effective concentration” or its abbreviation “EC” are used interchangeably to refer to the concentration of an agent (e.g., an hIL2 mutein) in an amount sufficient to effect a change in a given parameter in a test system. The abbreviation “E” refers to the magnitude of a given biological effect observed in a test system when that test system is exposed to a test agent. When the magnitude of the response is expressed as a factor of the concentration (“C”) of the test agent, the abbreviation “EC” is used. In the context of biological systems, the term Emax refers to the maximal magnitude of a given biological effect observed in response to a saturating concentration of an activating test agent. When the abbreviation BC is provided with a subscript (e.g., EC₄₀, EC₅₀, etc.) the subscript refers to the percentage of the Emax of the biological observed at that concentration. For example, the concentration of a test agent sufficient to result in the induction of a measurable biological parameter in a test system that is 30% of the maximal level of such measurable biological parameter in response to such test agent, this is referred to as the “EC₃₀” of the test agent with respect to such biological parameter. Similarly, the term “EC₁₀₀” is used to denote the effective concentration of an agent that results the maximal (100%) response of a measurable parameter in response to such agent. Similarly, the term EC₅₀ (which is commonly used in the field of pharmacodynamics) refers to the concentration of an agent sufficient to results in the half-maximal (50%) change in the measurable parameter. The term “saturating concentration” refers to the maximum possible quantity of a test agent that can dissolve in a standard volume of a specific solvent (e.g., water) under standard conditions of temperature and pressure. In pharmacodynamics, a saturating concentration of a drug is typically used to denote the concentration sufficient of the drug such that all available receptors are occupied by the drug, and EC₅₀ is the drug concentration to give the half-maximal effect. The EC of a particular effective concentration of a test agent may be abbreviated with respect to the with respect to particular parameter and test system.

Extracellular Domain: As used herein the term “extracellular domain” or its abbreviation “ECD” refers to the portion of a cell surface protein (e.g. a cell surface receptor) which is outside of the plasma membrane of a cell. The term “ECD” may include the extra-cytoplasmic portion of a transmembrane protein or the extra-cytoplasmic portion of a cell surface (or membrane associated protein).

Identity: As used herein, the term “percent (%) sequence identity” or “substantially identical” used in the context of nucleic acids or polypeptides, refers to a sequence that has at least 50% sequence identity with a reference sequence. Alternatively, percent sequence identity can be any integer from 50% to 100%. In some embodiments, a sequence has at least 50%, 55%, 60%, 65%, 70%, 75%, 80%, 85%, 90%, 91%, 92%, 93%, 94%, 95%, 96%, 97%, 98%, or 99% sequence identity to the reference sequence as determined with BLAST using standard parameters, as described below. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A comparison window includes reference to a segment of any one of the number of contiguous positions, e.g., a segment of at least 10 residues. In some embodiments, the comparison window has from 10 to 600 residues, e.g., about 10 to about 30 residues, about 10 to about 20 residues, about 50 to about 200 residues, or about 100 to about 150 residues, in which a sequence may be compared to a reference sequence of the same number of contiguous positions after the two sequences are optimally aligned. Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). The BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference sequence if the smallest sum probability in a comparison of the test amino acid sequence to the reference amino acid sequence is less than about 0.01, more preferably less than about 10⁻⁵, and most preferably less than about 10⁻²⁰.

As used herein, the term “interleukin 27 receptor” or “IL27R” refers to a heterodimeric receptor formed by subunits IL27Rα (IL27Rα) and glycoprotein 130 (gp130) and bound by the ligand IL27. The human sequence of IL27Rα is listed as UniProt ID NO. Q6UWB1. The human sequence of gp130 is listed as UniProt ID NO. Q13514.

Intracellular Signaling: As used herein, the terms “intracellular signaling” and “downstream signaling” are used interchangeably to refer to the to the cellular signaling process that is caused by the interaction of the intracellular domains (ICDs) of two or more cell surface receptors that are in proximity of each other. In receptor complexes via the JAK/STAT pathway, the association of the ICDS of the receptor subunits brings the JAK domains of the ICDs into proximit which initiates a phosphorylation cascade in which STAT molecules are phosphorylated and translocate to the nucleus associating with particular nucleic acid sequences resulting in the activation and expression of particular genes in the cell. The binding molecules of the present disclosure provide intraceullar signaling characteristic of the IL-27 receptor when activated by its natural cognate IL27. To measure downstream signaling activity, a number of methods are available. For example, in some embodiments, one can measure JAK/STAT signaling by the presence of phosphorylated receptors and/or phosphorylated STATs. In other embodiments, the expression of one or more downstream genes, whose expression levels can be affected by the level of downstream signaling caused by the binding molecule, can also be measured.

Ligand: As used herein, the term “ligand” refers to a molecule that exhibits specific binding to a receptor and results in a change in the biological activity of the receptor so as to effect a change in the activity of the receptor to which it binds. In one embodiment, the term “ligand” refers to a molecule, or complex thereof, that can act as an agonist or antagonist of a receptor. As used herein, the term “ligand” encompasses natural and synthetic ligands. “Ligand” also encompasses small molecules, e.g., peptide mimetics of cytokines and peptide mimetics of antibodies. The complex of a ligand and receptor is termed a “ligand-receptor complex.”

As used herein, the term “linker” refers to a linkage between two elements, e.g., protein domains. A linker can be a covalent bond or a peptide linker. The term “bond” refers to a chemical bond, e.g., an amide bond or a disulfide bond, or any kind of bond created from a chemical reaction, e.g., chemical conjugation. The term “peptide linker” refers to an amino acid or polypeptide that may be employed to link two protein domains to provide space and/or flexibility between the two protein domains.

Modulate: As used herein, the terms “modulate”, “modulation” and the like refer to the ability of a test agent to affect a response, either positive or negative or directly or indirectly, in a system, including a biological system or biochemical pathway.

Multimerization: As used herein, the term “multimerization” refers to two or more cell surface receptors, or domains or subunits thereof, being brought in close proximity to each other such that the receptors, or domains or subunits thereof, can interact with each other and cause intracellular signaling.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. The terms “immediately N-terminal” or “immediately C-terminal” are used to refers to a position of a first amino acid residue relative to a second amino acid residue where the first and second amino acid residues are covalently bound to provide a contiguous amino acid sequence.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the Operably Linked: The term “operably linked” is used herein to refer to the relationship between nucleic acid sequences encoding differing functions when combined into a single nucleic acid sequence that, when introduced into a cell, provides a nucleic acid which is capable of effecting the transcription and/or translation of a particular nucleic acid sequence in a cell. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, certain genetic elements such as enhancers need not be contiguous with respect to the sequence to which they provide their effect.

Partial Agonist: As used herein, the term “partial agonist” refers to a molecule that specifically binds that bind to and activate a given receptor but possess only partial activation the receptor relative to a full agonist. Partial agonists may display both agonistic and antagonistic effects. For example, when both a full agonist and partial agonist are present, the partial agonist acts as a competitive antagonist by competing with the full agonist for the receptor binding resulting in net decrease in receptor activation relative to the contact of the receptor with the full agonist in the absence of the partial agonist. Clinically, partial agonists can be used to activate receptors to give a desired submaximal response when inadequate amounts of the endogenous ligand are present, or they can reduce the overstimulation of receptors when excess amounts of the endogenous ligand are present. The maximum response (Emax) produced by a partial agonist is called its intrinsic activity and may be expressed on a percentage scale where a full agonist produced a 100% response. A In some embodiments, the IL-27 binding molecule has a reduced Eₘₐₓ compared to the Eₘₐₓ caused by IL-27. Eₘₐₓ reflects the maximum response level in a cell type that can be obtained by a ligand (e.g., a binding molecule described herein or the native cytokine (e.g., IL-27)). In some embodiments, the IL-27 binding molecule described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by IL-27. In other embodiments, the Eₘₐₓ of the IL-27 binding molecule described herein is greater (e.g., at least 1%, 5%, 10%, 15%, 20%, 25%, 30%, 35%, 40%, 45%, or 50% greater) than the Ex of the natural ligand, IL-27. In some embodiments, by varying the linker length of the IL-27 binding molecule, the Eₘₐₓ of the IL-27 binding molecule can be changed. The IL-27 binding molecule can cause Eₘₐₓ in the most desired cell types, and a reduced Emu in other cell types.

Polypeptide: As used herein the terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments) and the like.

As used herein the terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition from a present its state to a more deleterious state.

Proximity: As used herein, the term “proximity” refers to the spatial proximity or physical distance between two cell surface receptors, or domains or subunits thereof, after a binding molecule described herein binds to the two cell surface receptors, or domains or subunits thereof. In some embodiments, after the binding molecule binds to the cell surface receptors, or domains or subunits thereof, the spatial proximity between the cell surface receptors, or domains or subunits thereof, can be, e.g., less than about 500 angstroms, such as e.g., a distance of about 5 angstroms to about 500 angstroms. In some embodiments, the spatial proximity amounts to less than about 5 angstroms, less than about 20 angstroms, less than about 50 angstroms, less than about 75 angstroms, less than about 100 angstroms, less than about 150 angstroms, less than about 250 angstroms, less than about 300 angstroms, less than about 350 angstroms, less than about 400 angstroms, less than about 450 angstroms, or less than about 500 angstroms. In some embodiments, the spatial proximity amounts to less than about 100 angstroms. In some embodiments, the spatial proximity amounts to less than about 50 angstroms. In some embodiments, the spatial proximity amounts to less than about 20 angstroms. In some embodiments, the spatial proximity amounts to less than about 10 angstroms. In some embodiments, the spatial proximity ranges from about 10 to 100 angstroms, from about 50 to 150 angstroms, from about 100 to 200 angstroms, from about 150 to 250 angstroms, from about 200 to 300 angstroms, from about 250 to 350 angstroms, from about 300 to 400 angstroms, from about 350 to 450 angstroms, or about 400 to 500 angstroms. In some embodiments, the spatial proximity amounts to less than about 250 angstroms, alternatively less than about 200 angstroms, alternatively less than about 150 angstroms, alternatively less than about 120 angstroms, alternatively less than about 100 angstroms, alternatively less than about 80 angstroms, alternatively less than about 70 angstroms, or alternatively less than about 50 angstroms.

Receptor: As used herein, the term “receptor” refers to a polypeptide having a domain that specifically binds a ligand that binding of the ligand results in a change to at least one biological property of the polypeptide. In some embodiments, the receptor is a “soluble” receptor that is not associated with a cell surface. In some embodiments, the receptor is a cell surface receptor that comprises an extracellular domain (ECD) and a membrane associated domain which serves to anchor the ECD to the cell surface. In some embodiments of cell surface receptors, the receptor is a membrane spanning polypeptide comprising an intracellular domain (ICD) and extracellular domain (ECD) linked by a membrane spanning domain typically referred to as a transmembrane domain (TM). The binding of the ligand to the receptor results in a conformational change in the receptor resulting in a measurable biological effect. In some instances, where the receptor is a membrane spanning polypeptide comprising an ECD, TM and ICD, the binding of the ligand to the ECD results in a measurable intracellular biological effect mediated by one or more domains of the ICD in response to the binding of the ligand to the ECD. In some embodiments, a receptor is a component of a multi-component complex to facilitate intracellular signaling. For example, the ligand may bind a cell surface molecule having not associated with any intracellular signaling alone but upon ligand binding facilitates the formation of a multimeric complex that results in intracellular signaling.

Recombinant: As used herein, the term “recombinant” is used as an adjective to refer to the method by a polypeptide, nucleic acid, or cell that was modified using recombinant DNA technology. A recombinant protein is a protein produced using recombinant DNA technology and may be designated as such using the abbreviation of a lower case “r” (e.g., rhIL2) to denote the method by which the protein was produced. Similarly, a cell is referred to as a “recombinant cell” if the cell has been modified by the incorporation (e.g., transfection, transduction, infection) of exogenous nucleic acids (e.g., ssDNA, dsDNA, ssRNA, dsRNA, mRNA, viral or non-viral vectors, plasmids, cosmids and the like) using recombinant DNA technology. The techniques and protocols for recombinant DNA technology are well known in the art such as those can be found in Sambrook, et al. (1989) Molecular Cloning: A Laboratory Manual (2d ed., Cold Spring Harbor Laboratory Press, Plainview, N.Y.) and other standard molecular biology laboratory manuals.

Response: The term “response,” for example, of a cell, tissue, organ, or organism, encompasses a quantitative or qualitative change in a evaluable biochemical or physiological parameter, (e.g., concentration, density, adhesion, proliferation, activation, phosphorylation, migration, enzymatic activity, level of gene expression, rate of gene expression, rate of energy consumption, level of or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors. In contrast, the terms “inhibition”, “down-regulation” and the like refer to the opposite effects.

Single Domain Antibody (sdAb): The term “single-domain antibody” or “sdAbs,” refers to an antibody having a single (only one) monomeric variable antibody domain. A sdAb is able to bind selectively to a specific antigen. A V_{H}H antibody, further defined below, is an example of a sdAb.

Specifically Binds: As used herein, the term “specifically bind” refers to the degree of selectivity or affinity for which one molecule binds to another. In the context of binding pairs (e.g., a binding molecule described herein/receptor, a ligand/receptor, antibody/antigen, antibody/ligand, antibody/receptor binding pairs), a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the affinity of the first molecule for the second molecule is at least two-fold greater, alternatively at least five times greater, alternatively at least ten times greater, alternatively at least 20-times greater, or alternatively at least 100-times greater than the affinity of the first molecule for other components present in the sample.

Stably Associated: As used herein, the term “stably associated” or “in stable association with” are used to refer to the various means by which one molecule (e.g., a polypeptide) may be associated with another molecule over an extended period of time. The stable association of one molecule to another may be effected by a variety of means, including covalent bonding and non-covalent interactions. In some embodiments, stable association of two molecules may be effected by covalent bonds such as peptide bonds. In other embodiments, stable association of two molecules may be effected b non-covalent interactions. Examples of non-covalent interactions which may provide a stable association between two molecules include electrostatic interactions (e.g., hydrogen bonding, ionic bonding, halogen binding, dipole-dipole interactions, Van der Waals forces and π-effects including cation-π interactions, anion-π interactions and π-π interactions) and hydrophobilic/hydrophilic interactions. In some embodiments, the stable association of sdAbs of the bivalent binding molecules of the present disclosure may be effected by non-covalent interactions. In one embodiment, the non-covalent stable association of the sdAbs of the bivalent binding molecules may be achieved by conjugation of the sdAbs to “knob-into-hole” modified Fe monomers. An Fe “knob” monomer stably associates non-covalently with an Fc “hole” monomer. Conjugation of a first sdAb which specifically binds to the extracellular domain of a first subunit of a heterodimeric receptor to an “Fc knob” monomer and conjugation of an second sdAb which specifically binds to the extracellular domain of a second subunit of a heterodimeric receptor to an “Fc hole” monomer provides stable association of the first and second sdAbs. The knob-into-hole modification is more fully described in Ridgway, et al. (1996) Protein Engineering 9 (7): 617-621 and U.S. Pat. No. 5,731,168, issued Mar. 24, 1998, U.S. Pat. No. 7,642,228, issued Jan. 5, 2010, U.S. Pat. No. 7,695,936, issued Apr. 13, 2010, and U.S. Pat. No. 8,216,805, issued Jul. 10, 2012. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”) and ii) in the CH3 domain of a second heavy chain, an amino acid residue is replaced with an amino acid residue having a smaller side chain (e.g., alanine or threonine), thereby generating a cavity (“hole”) within at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 on one chain and Y349 on the second chain which results in a stabilizing disulfide bridge between the two antibody heavy chains in the Fc region (Carter, et al. (2001) Immunol Methods 248, 7-15). The knob-into-hole format is used to facilitate the expression of a first polypeptide (e.g., an IL27Rα binding sdAb) on a first Fe monomer with a “knob” modification and a second polypeptide on the second Fe monomer possessing a “hole” modification to facilitate the expression of heterodimeric polypeptide conjugates.

Subject: The terms “recipient”, “individual”, “subject”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is a human being.

Substantially; As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher of a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Suffering From: As used herein, the term “suffering from” refers to a determination made by a physician with respect to a subject based on the available information accepted in the field for the identification of a disease, disorder or condition including but not limited to X-ray, CT-scans, conventional laboratory diagnostic tests (e.g., blood count), genomic data, protein expression data, immunohistochemistry, that the subject requires or will benefit from treatment. The term suffering from is typically used in conjunction with a particular disease state such as “suffering from a neoplastic disease” refers to a subject which has been diagnosed with the presence of a neoplasm.

Therapeutically Effective Amount: As used herein, the term The phrase “therapeutically effective amount” is used in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it may be adjusted in connection with a dosing regimen and in response to diagnostic analysis of the subject's condition, and the like. The parameters for evaluation to determine a therapeutically effective amount of an agent are determined by the physician using art accepted diagnostic criteria including but not limited to indicia such as age, weight, sex, general health, ECOG score, observable physiological parameters, blood levels, blood pressure, electrocardiogram, computerized tomography, X-ray, and the like. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, modification of biomarker levels, increase in duration of survival, extended duration of progression free survival, extension of the time to progression, increased time to treatment failure, extended duration of event free survival, extension of time to next treatment, improvement objective response rate, improvement in the duration of response, and the like that that are relied upon by clinicians in the field for the assessment of an improvement in the condition of the subject in response to administration of an agent.

Treat: The terms “treat”, “treating”, treatment” and the like refer to a course of action (such as administering a binding molecule described herein, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

VHH: As used herein, the term “V_{H}H” is a type of sdAb that has a single monomeric heavy chain variable antibody domain. Such antibodies can be found in or produced from Camelid mammals (e.g., camels, llamas) which are naturally devoid of light chains V_{H}Hs can be obtained from immunization of camelids (including camels, llamas, and alpacas (see, e.g., Hamers-Casterman, et al. (1993) Nature 363:446-448) or by screening libraries (e.g., phage libraries) constructed in V_{H}H frameworks. Antibodies having a given specificity may also be derived from non-mammalian sources such as V_{H}Hs obtained from immunization of cartilaginous fishes including, but not limited to, sharks. In a particular embodiment, a V_{H}H in a bispecific V_{H}H² binding molecule described herein binds to a receptor (e.g., the first receptor or the second receptor of the natural or non-natural receptor pairs) if the equilibrium dissociation constant between the V_{H}H and the receptor is greater than about 10⁻⁶ M, alternatively greater than about 10⁻⁸ M, alternatively greater than about 10⁻¹⁰ M, alternatively greater than about 10⁻¹¹ M, alternatively greater than about 10⁻⁸⁰ M, greater than about 10⁻¹² M as determined by, e.g., Scatchard analysis (Munsen, et al. 1980 Analyt. Biochem. 107:220-239). Standardized protocols for the generation of single domain antibodies from camelids are well known in the scientific literature. See, e.g., Vincke, et al (2012) Chapter 8 in Methods in Molecular Biology, Walker, J. editor (Humana Press, Totowa NJ). Specific binding may be assessed using techniques known in the art including but not limited to competition ELISA, BIACORE® assays and/or KINEXA® assays. In some embodiments, a V_{H}H described herein can be humanized to contain human framework regions. Examples of human germlines that could be used to create humanized V_{H}Hs include, but are not limited to, VH3-23 (e.g., UniProt ID: P01764), VH3-74 (e.g., UniProt ID: A0A0BAJ1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

V_{H}H²: As used herein, the term “V_{H}H²” and “bispecific V_{H}H²” and “V_{H}H dimer” refers to are used interchangeably to refer to a subtype of the binding molecules of the present disclosure wherein the first and second sdAbs are both VHHs and first V_{H}H binding to a first receptor, or domain or subunit thereof, and a second V_{H}H binding to a second receptor, or domain or subunit thereof.

Wild Type: As used herein, the term “wild type” or “WT” or “native” is used to refer to an amino acid sequence or a nucleotide sequence that is found in nature and that has not been altered by the hand of man.

### III. Il27 Receptor Binding Proteins

The IL27 receptor (IL27R) includes IL27Rα subunit (IL27Rα) and glycoprotein 130 subunit (gp130). Provided herein is an IL27R binding protein that specifically binds to IL27Rα and gp130. In some embodiments, the IL27R binding protein binds to a mammalian cell expressing both IL27Rα and gp130. In some embodiments, the IL27R binding protein can be a bispecific V_{H}H² as described below.

The IL27R binding protein can be a bispecific V_{H}H² that has a first V_{H}H binding to IL27Rα (an anti-IL27Rα V_{H}H antibody) and a second V_{H}H binding to gp130 (an anti-gp130 V_{H}H antibody) and causes the dimerization of the two receptor subunits and downstream signaling when bound to a cell expressing IL27Rα and gp130, e.g., a CD8⁺ T cells, a CD4⁺ T cells, and/or a T regulatory (Treg) cell.

A V_{H}H is a type of single-domain antibody (sdAb) containing a single monomeric variable antibody domain. Like a full-length antibody, it is able to bind selectively to a specific antigen. The complementary determining regions (CDRs) of V_{H}Hs are within a single-domain polypeptide. V_{H}Hs can be engineered from heavy-chain antibodies found in camelids.

An exemplary V_{H}H has a molecular weight of approximately 12-15 kDa which is much smaller than traditional mammalian antibodies (150-160 kDa) composed of two heavy chains and two light chains. V_{H}Hs can be found in or produced from Camelidae mammals (e.g., camels, llamas, dromedary, alpaca, and guanaco) which are naturally devoid of light chains. Descriptions of sdAbs and V_{H}HS can be found in, e.g., De Greve et al., Curr Opin Biotechnol. 61:96-101, 2019; Ciccarese, et al., Front Genet. 10:997, 2019; Chanier and Chames, Antibodies (Basel) 8 (1), 2019; and De Vlieger et al., Antibodies (Basel) 8 (1), 2018.

To prepare a binding protein that is a bispecific V_{H}H², in some embodiments, the two V_{H}Hs can be synthesized separately, then joined together by a linker. Alternatively, the bispecific V_{H}H² can be synthesized as a fusion protein. V_{H}Hs having different binding activities and receptor targets can be paired to make a bispecific V_{H}H². The binding proteins can be screened for signal transduction on cells carrying one or both relevant receptors.

In some embodiments, a bispecific VH2 comprises:

A first V_{H}H antibody comprising a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR1 from a row of Table 1A; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR2 from the same row of Table 1A; and a CDR3 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR3 from the same row of Table 1A; and
a second V_{H}H antibody comprising a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR4 from the same row of Table 1A; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of a CDR5 from the same row of Table 1A; and a CDR3 having at least 90% (e.g., 91%, 92%, 93%, 94%, 95%, 96%, 97%, 98%, 99%, or 100%) sequence identity, or having 0, 1, 2, or 3 amino acid changes optionally conservative amino acid changes relative, to the sequence of a CDR6 from the same row of Table 1A. In some embodiments, the bispecific V_{H}H² comprises a sequence at least 90% (e.g., 91%, 92%, 93%, 94%, 95%, 96%, 97%, 98%, 99%, or 100%) identical to any one of the dual VHH dimer sequences shown in Table 1A.

In some embodiments, a V_{H}H described herein can be humanized to contain human framework regions. Examples of human germlines that could be used to create humanized V_{H}Hs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

Lengthy table referenced here
US20250179198A1-20250605-T00001
Please refer to the end of the specification for access instructions.

In some embodiments, an IL27R binding protein described herein (e.g., in Table 1A) is encoded by an isolated nucleic acid that is substantially identical to a sequence of any one of Table 1B below. In some embodiments, an IL27R binding protein described herein (e.g., an IL27R binding protein comprising a sequence of Table 1A) is encoded by an isolated nucleic acid comprising a sequence having at least 90% (e.g., 91%, 92%, 93%, 94%, 95%, 96%, 97%, 98%, 99%, or 100%) sequence identity to a sequence of Table 1B below.

TABLC 1B

| | SCQ ID | |
|---|---|---|
| Name | NO: | Sequence |
| DR591- | 955 | CAGGTTCAGCTCCAGGAGAGGGGCGGGGGCTCCGTGCAGGCTGGCGGATCACTG |
| hIL27 | | CGTCTGTCCTGTACTGCGAGCGGCGCTATCGCGAGCGGGTACATTGATAGCAGA |
| Ra_VH | | TGGTGTATGGCTTGGTTCCGTCAGGCACCAGGCAAGGAGCGTGAGGGCGTGGCA |
| H1 | | GCTATCTGGCCTGGAGGGGGTCTCACCGTGTACGCCGACTCCGTGAAGGGCCGT |
| | | TTCACAATTAGTAGAGACCATGCCAAGAATACGCTCTATGTTCAGATGAATAAC |
| | | CTGAAACCCGAGGATACGGCTATGTACTATTGTGCTGCGGGATCACCCGGGATG |
| | | TGCCCGTCTCTGGAGTTTGGATTCGACTATTGGGGACAGGGGACCCAGGTCACC |
| | | GTCAGTTCCGGTGGGGGCTCCCAAGTCCAGCTCCAGGAGTCCGGGGGGGGCTTG |
| | | GTCCAGCCCGGAGGCTCCCTCCGCCTGAGTTGTGCTGCCAGCGGATTCACTTTT |
| | | TCCAGTTACCCTATGTCCTGGGTGAGACAGGCCCCTGGGAAGGGCCTGGAGTGG |
| | | ATCTCTACCATCAGGGGGGGGGGGGATACCACTCTGTATGCCGACTCAGTGAAG |
| | | GGCAGGTTCACGTCTTCCCGCGATAACGCTAAGAATACTCTCTATCTGCAACTC |
| | | AATTCCCTGAAGACAGAGGACGCAGCTATCTACTATTGTGCTAAGCGTATTGAT |
| | | TGCAACAGCGGATACTGCTACCGTAGGAACTATTGGGGCCAGGGCACCCAGGTG |
| | | ACTGTGTCCTCC |
| DR591- | 956 | CAGGTACAGCTCCAGGAGTCTGGTGGAGGCTCTGTGCAAGCAGGGGGTAGCCTG |
| hIL27 | | AGGCTGTCATGCACAGCCTCCGGTGCCATCGCCAGCGGTTACATTGACAGCAGG |
| Ra_VH | | TGGTGTATGGCTTGGTTCAGGCAGGCCCCCGGCAAAGAACGTGAAGGCGTGGCC |
| | | GCTATCTGGCCCGGCGGTGGACTGACTGTCTATGCCGATAGTGTGAAAGGCAGA |
| | | TTCACCATCTCTCGGGATCACGCGAAGAATACACTGTACCTTCAGATGAATAAC |
| | | CTGAAGCCTGAGGACACCGCCATGTACTATTGCGCTGCGGGCTCTCCGAGGATG |
| | | TGCCCCAGCCTGGAGTTCGGGTTCGATTATTGGGGTCAGGGCACCCAAGTGACT |
| | | GTGAGCAGTGGGGGATCTGGCGGGTCCGGTGGCTCTGGTCAGGTGCAGCTGCAA |
| | | GAGTGTGGAGGGGGCGTCGTACAACCTGGAGGTAGTCTGCGCCTCAGTTGTGCT |
| | | GCATCTGGCTTCACCTTCAGCTCCTATCCCATGTCTTGGGTTCGGCAGGCTCCC |
| | | GGCAAGGGGCTGGAGTGGATCTCCACCATCAGCGCGGGGGGGGACACAACTCTG |
| | | TACGCCGATTCTGTGAAGGGGCGTTTCACAAGGTCAAGAGATAACGCAAAAAAC |
| | | ACGCTCTACCTCCAGGTTAATTCATTGAAGACCGAAGACGCAGCTATTTATTAC |
| | | TGCGCTAAGCGCATCGACTGTAACTCCGGCTACTGCTATAGAAGGAATTACTGG |
| | | GGCCAGGGAACACAGGTTACCGTGAGTTCA |
| DR591- | 957 | CAGGTGCAGCTCCAGGAGTCCGGTGGCGGAAGCGTCCAGGCTGGCGGAAGTCTG |
| hIL27 | | AGGCTCTCATGCACCGCCTCTGGTGCCATCGCCAGCGGATACATCGACTCCAGA |
| Ra_VH | | TGGTGTATGGCTTGGTTTAGGCAAGCCCCTGGCAAGGAAAGGGAGGGGGTTGCT |
| H2 | | GCCATCTGGCCCGGCGGAGGCGTGACGGTGTATGCAGACAGCGTCAAAGGGAGA |
| | | TTCACCATTTCTCGCGACCACGCTAAGAACACCCTTTACTTGCAGATGAATAAC |
| | | CTCAAGCCCGAGGACACCGCCATGTATTACTGCGCCGCTGGTTCACCGAGGATG |
| | | TGCCCGAGCCTGGAGTTCGGCTTCGACTACTGGGGCCAAGGTACACAAGTGACT |
| | | GTGTCCAGGGGCGGGGGTAGCCAGGTTCAGCTGCAAGAGAGCGGCGGTGGATTG |
| | | GTGCAGCCAGGCGGTAGCCCGCGCCTGTCTTGCGCGGCCAGCGGATTCACATTC |
| | | TCCCTGAGCGGCATGTCTTGGGTGCGCCAAGCTCCGGGCAAAGGGCTGGAGTGG |
| | | GTCAGCGCCATCTCTAGCGGAGGTGCCTCTACGTATTACACAGATTCCGTTAAG |
| | | GGCCGCTTCACTATCAGCCGCGATAACGCCAAGAACATCCTGTATCTCCAGTTG |
| | | AACAGCCTGAAAACGGAGGACACAGCCATGTACTATTGGGCTAAAGGGGGTTCA |
| | | GGCTATGGTGACGCTTCTCGCATGACCTCACCCGGCTCACAGGGAACCCAGGTG |
| | | ACAGTGTCAAGT |
| DR591- | 958 | CAGGTGCAGCTGCAAGAGTCCGGCGGAGGTAGCGTTCAAGCCGGAGGCTCACTG |
| hIL27 | | AGACTGTCTTGCACCGCCTCTGGTGCAATCGCGTCTGGCTATATTGATTCTCGC |
| Ra_VH | | TGGTGTATGGCGTGGTTTAGGCAAGCGCCTGGGAAAGAGCGTGAAGGAGTTGCA |
| H2 | | GCCATCTGGCCGGGAGGCGGACTGACCGTGTACGCTGACTCTGTGAAGGGCAGA |
| | | TTCACCATCAGTCGTGACCACGCCAAGAACACTCTGTACCTTCAGATGAATAAC |
| | | CTCAAACCAGAGGACACAGCTATGTACTATTGTGCGGCTGGATCTCCGCGCATG |
| | | TGCCCTAGTCTGGAGTTCGGTTTTGATTACTGGGGCCAGGGAACTCAAGTGACC |
| | | GTGTCTAGCGGCGGTTCTGGAGGCAGTGGTGGGTCAGGTCAGGTTCAGCTCCAA |
| | | GAGTCAGGTGGGGGACTCGTGCAACCAGGGGGCTCCCTGCGCCTGTCTTGCGCT |
| | | GCCTCCGGCTTCACTTTCAGCCTGAGCGGTATGTCCTGGGTTCGCCAGGGTCCT |
| | | GGTAAAGGGCTGGAGTGGGTTAGTGCTATCAGCTCCGGGGGAGCTTCTACCTAT |
| | | TACACCGACTCTGTTAAGGGTCGCTTCACTATCAGCCGCGACAACGCCAAAAAT |
| | | ATTTTGTATCTGCAACTCAACTCCTTGAAGACAGAGGACACCGCGATGTATTAC |
| | | TGCGCCAAGGGAGGTAGCGGCTACGGCGATGCTAGTCGTATGACGAGCCCCGGC |
| | | TCTCAGGGTACACAAGTCACCGTAAGCTCC |
| DR591- | 959 | CAGGTTCAGTTGCAGGAGTCTGGCGGAGGGAGCGTTCAGGCTGGCGGTTCCCTG |
| hIL27 | | AGGCTCTCTTGCACAGCCTCTGGCGCTATCGCCTCAGGATATATTGATTCCCGT |
| Ra_VH | | TGGTGTATGGCATGGTTCCGTCAGGCCCCTGGCAAGGAACGCGAGGGAGTCGCT |
| H3 | | GCCATCTGGCCCGGCGGGGGCGTGACAGTCTACGCTGATTCAGTTAAGGGCCGC |
| | | TTCACTATCAGCCGCGACCACGCTAAAAACACGCTGTACCTGCAAATGAATAAC |
| | | CTCAAACCAGAGGACACTGCAATGTATTACTGCGCCGCAGGCTCTCCCAGAATG |
| | | TGCCCGAGCCTGGAGTTTGGTTTCGATTACTGGGGTCAGGGCACGCAGGTAACC |
| | | GTGAGTTCTGGTGGAGGCTCCCAGGTGCAGCTCCAAGAGTCCGGGGGAGGCTCT |
| | | GTGCAAGCAGGCGGAAGCCTGAGATTGTCCTGCGTGGCCAGCGGTTATGTGTCC |
| | | TGTGATTACTTCCCGCCTTCTTGGTATCGGCAGGCCCCAGGCAAGGAGCGCGAG |
| | | TTCGTCTCCATCATTGATGGCACCGGATCAACAAGCTACGCTGCGTCCGTGAAG |
| | | GGTCGGTTTACAGCAAGCGAGGATAAGGGAAAGAACATCGCGTACCTCCAGATG |
| | | AACAGCCTGAAGCCTGAAGACACTGCTATGTATTACTGTAAGGCTAGTTGTGTG |
| | | CGGGGCCGCGCAGTTTCTGAATATTGGGGTCAGGGAACACAAGTAACCGTGTCT |
| | | TCA |
| DR591- | 960 | CAGGTCCAGGTCCAGGAATCAGGAGGTGGCTCCGTACAGGCCGGAGGGAGTCTC |
| hIL27 | | CGCCTCTCCTGTACTGCCAGTGGTGCCATCGCAAGCGGCTATATCGACAGCCGT |
| Ra_VH | | TGGTGCATGGCCTGGTTCAGGCAGGCACCCGGCAAAGAGAGAGAAGGAGTCGCT |
| H3 | | GCCGATCTGGCCGGGGGAGGTTTGACCGTCTACGCCGACAGCGTCAAGGGCAGA |
| | | TTTACTATCAGCCGTGACCACGCGAAGAACACCCTCTATCTTCAGATGAATAAC |
| | | CTCAAACCTGAGGATACCGCCATGTATTACCGCGCAGCCGGGAGCCCTCGGATG |
| | | TGCCCTAGCGTGGAGTTCGGCTTCGACTACTGGGGCCAAGGGACACAGGTAACA |
| | | GTTAGTAGCGGTGGCTCCGGTGGCTCCGGGGGTTCTGGTCAGGTCCAGCTCCAG |
| | | GAGTCTGGAGGCGGTTCAGTGCAGGCTGGCGGTTCACTGCGCCTCAGCTGCGTG |
| | | GCCTCCGGCTACGTATCATGCGACTATTTCCTGCCCTCGTGGTATCGCCAGGCC |
| | | CCCGGAAAAGAGAGGGAGTTTGTGTCTATTATCGACGGGACCGGAAGCACCAGG |
| | | TACGCTGCCTCAGTGAAGGGTCGCTTCACCGCCTCCGAGGATAAGGGCAAGAAC |
| | | ATTGCCTACTTGCAGATGAACTCATTGAAACCGGAAGACACTGCGATGTATTAC |
| | | TGTAAGGCTTCTTGCGTTCGCGGACGTGCTGTGAGCGAGTACTGGGGCCAAGGG |
| | | ACCCAAGTGACGGTCTCTTCT |
| DR591- | 961 | CAGGTGCAACTTCAGGAGTCTGGCGGTGGCAGCGTGCAGGCCGGGGGCTCCCTG |
| hIL27 | | CGGCTGTCATGTACGGCCTCCGGCGCTATTGCCAGCGGTTACATTGACTCCAGG |
| Ra_VH | | TGGTGCATGGCCTGGTTCCGTCAGGCTCCGGGCAAGGAGAGGGAAGGCGTGGCA |
| H4 | | GCCATCTGGCCCGGCGGGGGCCTCACCGTTTATGCCGATTCCGTGAAGGGACGT |
| | | TTTACAATCAGCCGTGACCATGCTAAGAACACCCTGTATTTGCAGATGAATAAC |
| | | CTGAAACCCGAGGACACCGCCATGTATTACTGCGCCGCAGGCAGCCCCCGTATG |
| | | TGCCCGTCCCTGGAGTTTGGCTTCGATTACTGGGGCCAGGGGACCCAGGTGACA |
| | | GTCAGCTCCGGGGGTGGCTCTCAGGTGCAACTTCAGGAATCCGGTGGAGGTCTG |
| | | GTGCAGCCGGGAGAGTCTCTGCGTCTGTCATGCACTGCCTCAGGCTTCACGTTT |
| | | AGTAACTATGCTATGTCCTGGGTGAGGCAAGCTCCTGGAAAGGGCCTCGAATGG |
| | | GTGTCAGGAATCAACGTGGCCTACGGCATTACTTCTTATGCCGATTCTGTTAAG |
| | | GGTCGCTTCACTATCTCAAGAGACAACACTAAAAACACTCTTTACTTGCAGCTG |
| | | AACAGCCTGAAAACAGAGGACACAGCCATTTATTACTGCGTGAAGCACTCTGGC |
| | | ACAACCATCCCACGCGGTTTCATTTCATACACGAAGCGGGGCCAGGGGACCCAG |
| | | GTGACTGTGAGCAGC |
| DR591- | 962 | CAGGTCCAACTTCAGGAATCTGGAGGTGGCTCAGTACAGGCTGGAGGCTCCCTG |
| hIL27 | | AGACTGAGCTGCACCGCCTCAGGAGCAATCGCATCCGGCTATATCGACTCCCGT |
| Ra_VH | | TGGTGTATGGCCTGGTTTCGCCAGGCCCCCGGGAAGGAGCGTGAGGGCGTGGCC |
| H4 | | GCTATCTGGCCAGGCGGGGGCCTGACCGTGTACGCTGACAGTGTTAAAGGAAGG |
| | | TTCACAATTTCCCGCGATCACGCTAAGAATACATTGTATCTTCAGATGAATAAC |
| | | CTCAAGCCAGAGGACACCGCCATGTATTACTGCGCAGCCGGTTCACCCCGCATG |
| | | TGCCCCAGCCTGGAGTTCGGCTTCGACTACTGGGGACAGGGCACCCAGGTTACC |
| | | GTGTCTTCCGGGGGTTCTGGCGGATCTGGTGGCTCCGGGCAGGTGCAGCTCCAG |
| | | GAATCTGGTGGGGGCCTGGTTCAGCCGGGTGAAAGCCTGAGGCTGTCTTGCACC |
| | | GCCTCTGGTTTCACCTTCAGCAACTATGCGATGTCTTGGGTGCGGCAAGCACCG |
| | | GGTAAGGGCGTGGAGTGGGTGAGCGGCATCAACGTGGCCTACGGTATTACAAGC |
| | | TACGCCGATTCCGTTAAAGGTCGTTTCACCATCTCTAGGGACAACACAAAGAAC |
| | | ACACTGTACTTGCAGCTGAACTCTCTGAAGACCGAAGATACCGCCATCTATTAC |
| | | TGTGTGAAGCATTCCGGCACAACCATCCCTCGCGGCTTCATCTCCTATACCAAG |
| | | CGCGGCCAGGGCACCCAGGTCACGGTTTCCCCC |
| DR591- | 963 | CAGGTGCAGCTGCAAGAGTCCGGTGGCGGGTCCGTCCAGGCCGGGGGCTCTCTG |
| hIL27 | | CGTCTTAGCTGCACCGCTTCTGGGGCCATTGCTTCTGGCTACATTGATTCCAGA |
| Ra_VH | | TGGTGTATGGCCTGGTTCCGCCAAGCGCCCGGCAAGGAGCGCGAGGGCGTGGCC |
| H5 | | GCTATCTGGCCCGGTGGAGGCCTTACCGTCTATGCTGACAGCGTGAAAGGACGC |
| | | TTCACCATCTCCCGCGACCACGCCAAAAACACCCTGTACTTGCAGATGAACAAT |
| | | TTGAAGCCCGAGGACACGGCCATGTATTACTGCGCCGCTGGTAGCCCCCGCATG |
| | | TGTCCGTCCCTGGAGTTCGGTTTCGACTACTGGGGGCAGGGCACCCAGGTGACA |
| | | GTCAGCTCTGGAGGGGGCTCCCAGGTGCAGCTCCAGGAGAGCGGCGGAGGCAGG |
| | | GTGCAGGCCGGGGGAAGCCTCAGGCTGAGCTGCACCGCGAGCGGGTACGTTTCC |
| | | TGTGATTATTTTCCCCCATCATGGTATCGCCAAGCGCCAGGAAAGGAGCGTGAG |
| | | TTCGTATCCGTGATTGACGGTACAGGTTCAACATCCTATGCGGCCTCTGTGAAG |
| | | GGGCGCTTCACCGCGAGCCAAGATAAGGGGAAGAACATCGCCTATTTGCAGATG |
| | | AACTCACTGAAGCCTGAGGACACCGCTATGTATTACTGCAAGGCTTCCTGTGTG |
| | | CGTGGCCGCGCCATCTCTGAGTACTGGGGGCAGGGCACCCAGGTGACCGTGTCC |
| | | TCC |
| DR591- | 964 | CAGGTGCAGGTGCAAGAATCTGGAGGCGGTAGGGTACAGGCAGGTGGCAGCCTG |
| hIL27 | | AGGCTGTCTTGTACTGCGTCCGGCGCTATCGCATCAGGATACATTGACTCACGT |
| Ra_VH | | TGGTGCATGGCCTGGTTTCGTCAGGCTCCAGGAAAGGAGCGTGAAGGCGTCGCT |
| H5 | | GCCATTTGGCCTGGTGGAGGCCTGACCGTGTATGCCGATAGCGTCAAGGGACGT |
| | | TTCACCATCAGCCGTGACCACGCTAAGAATACCCTGTACTTGCAAATGAACAAT |
| | | TTGAAGCCAGAGGACACTGCGATGTACTATTGCGCGGCAGGCTCCCCCAGGATG |
| | | TGTCCCAGCGTGGAGTTTGGTTTCGATTACTGGGGGCAGGGCACCCAGGTCACC |
| | | GTTAGCTCCGGGGGTTCTGGAGGTTCCGGCGGTTCAGGACAGGTGCAGCTTCAG |
| | | GAGTCTGGGGGAGGGTCCGTACAAGCCGGGGGCTCCTTGCGCCTGTCTTGCACA |
| | | GCTTCCGGCTATGTTAGTTGTGACTACTTCCTGCCTAGCTGGTATCGCCAAGCC |
| | | CCTGGCAAGGAGAGGGAGTTCGTTTCCGTGATTGATGGCACTGGGTCTACTTCC |
| | | TACGCTGCCAGTGTGAAGGGGCGCTTCACCGCTTCTCAAGACAAGGGCAAGAAC |
| | | ATCGCGTACCTCCAGATGAACAGCCTGAAGCCAGAAGATACCGCAATGTACTAT |
| | | TGTAAGGCCTCCTGCGTTCGGGGGGGGCCATCTCAGAATACTGGGGCCAGGGG |
| | | ACTCAGGTCACCGTGTCCAGT |
| DR591- | 965 | CAGGTGCAGTTGCAGGAATCTGGCGGAGGCTCCGTCCAGGCTGGTGGCAGTTTG |
| hIL27 | | CGTCTGTCCTGCACAGCTTCAGGCGCGATTGCCTCTGGGTACATTGATAGTAGA |
| Ra_VH | | TGGTGCATGGCTTGGTTCAGACAGGCTCCTGGAAAGGAGAGAGAGGGGGTGGCC |
| H6 | | GCGATCTGGCCCGGAGGCGGGCTGACTGTCTACGCCGATAGCGTTAAGGGTCGC |
| | | TTCACGATTTCTCGGGATCACGCCAAGAACACCCTGTACTTGCAAATGAATAAC |
| | | CTGAAACCCGAAGACACCGCAATGTATTACTGCGCCGCTGGTTCACCAAGGATG |
| | | TGCCCTTCCCTGGAGTTCGGTTTCGACTACTGGGGACAGGGCACCCAGGTAACC |
| | | GTGAGTAGCGGGGGGGGTAGTCAGGTGCAGCTTCAGGAGTCAGGCGGGGGCTTG |
| | | GTCCAACCTGGAGGGAGCCTCAGGCTCTCCTGTGCCGCTTCCGGCTTCAGTTTC |
| | | AGCTCCTATGCCATGAAGTGGGTAAGACAGGCCCCTGGCAAGGGCCTGGAGTGG |
| | | GTTTCCACCATTAGTAGCGGAGGCAGCTCAACCAACTACGCCGATTCCGTTAAG |
| | | GGCCGCTTTACGATCAGCCGTGACAATGCAAAGAACACCTTGTACCTCCAGCTG |
| | | AACTCCCTGAAAATTGAAGACACCGCAATGTATTACCGCGCCAAGGCAATCGTC |
| | | CCTACAGGGGCCACAATGGAGCGCGGTCAGGGAACTCAGGTGACCGTGTCCAGC |
| DR591- | 966 | CAGGTTCAGCTCCAGGAATCTGGAGGGGGAAGTGTGCAGGCTGGAGGTTCCCTC |
| hIL27 | | CGCCTCAGCTGCACTGCCAGCGGAGCTATCGCTTCCGGTTACATTGACAGCCGC |
| Ra_VH | | TGGTGTATGGCTTGGTTTCGCCAAGCGCCCGGCAAGGAACGCGAAGGGGTGGCG |
| H6 | | GCCATCTGGCCTGGGGGAGGCCTGACAGTGTACGCCGACAGCGTCAAGGGTAGG |
| | | TTTACTATCAGCAGAGACCACGCAAAGAACACCCTGTATCTGCAAATGAACAAT |
| | | CTGAAGCCGGAAGACACTGCAATGTATTACTGTGCTGCGGGCTCCCCCAGGATG |
| | | TGTCCCAGCCTGGAGTTCGGGTTTGACTACTGGGGTCAGGGGACGCAGGTGACC |
| | | GTCAGCTCCGGCGGTTCCGGGGGCAGCGGAGGCAGCGGACAGGTCCAGCTTCAG |
| | | GAGTCCGGTGGGGGCCTGGTCCAGCGTGGGGGCTCTCTGCGGCTGAGCTGTGCG |
| | | GCCAGCGGGTTTAGTTTCTCCAGTTACGCTATGAAATGGGTGCGTCAGGCACCG |
| | | GGTAAGGGACTCGAATGGGTCAGCACAATCAGTAGCGGAGGCAGGTCCACGAAC |
| | | TATGCTGATAGCGTGAAGGGAAGATTCACCATTAGCCGCGACAACGCCAAGAAT |
| | | ACATTGTATCTTCAGCTGAACAGCCTGAAGATCGAAGACACCGCGATGTATTAC |
| | | TGTGCTAAGGCCATCGTGCCCACTGGGGCCACAATGGAGAGGGGCCAGGGCACC |
| | | CAGGTCACGGTATCCAGC |
| DR591- | 967 | CAGGTCCAGTTGCAGGAAAGCGGAGGGGGATCTGTGCAGGCAGGTGGCTCCCTT |
| hIL27 | | CGCCTGAGCTGCACCGCAAGGGGGGCCATTGCGTCTGGATACATCGACTCCAGA |
| Ra_VH | | TGGTGCATGGCCTGGTTCCGCCAAGCGCCAGGCAAAGAGCGCGAAGGAGTGGCA |
| H7 | | GCCATCTGGCCTGGAGGGGGCGTCACAGTTTACGCTGACTCCGTTAAGGGCAGA |
| | | TTCACCATCTCCCGCGACCACGCCAAGAACACACTCTACCTCCAGATGAATAAC |
| | | CTGAAGCCCGAGGATACCGCCATGTATTACTGCGCAGCTGGAAGCCCACGTATG |
| | | TGCCCGTCCTTGGAGTTCGGCTTCGACTACTGGGGGCAAGGAACCCAGGTGACC |
| | | GTTTCCAGCGGGGGGGGATCTCAGGTCCAGTTGCAAGAGTCCGGGGGGGGGCTG |
| | | GTGCAGCCTGGGGGCTCTCCGCGCCTGTCCTGCGCAGCGAGCGGCTTCACCTTC |
| | | TCATCCTATCCTATGTCTTGGGTGCGTCAGGCCCCAGGTAAGGGACTGGAGTGG |
| | | ATTTCCACGATCAGTGCCGGTGGCGATACTACCCTCTACGCCGACTCTGTGAAA |
| | | GGACGGTTCACCAGTAGCCGTGACAACGCGAAGAACACATTGTATTTGCAGCTG |
| | | AACAGCCTCAAGACCGAAGATACCGCCATCTATTACTGGGCCAAGAGAATTGAT |
| | | TGTAACTCTGGATACTGCTACCGTCGCAACTACTGGGGACAGGGCACCCAGGTG |
| | | ACCGTCAGTTCC |
| DR591- | 968 | CAGGTGCAGTTGCAGGAATCCGGGGGAGGCAGTGTTCAGGCTGGCGGAAGCCTG |
| hIL27 | | CGCCTTTCCTGCACAGCCAGCGGGGCTATTGCCGCCGGCTACATTGATAGTCGT |
| Ra_VH | | TGGTGCATGGCCTGGTTCCGCCAAGCACCGGGTAAGGAGAGGGAAGGGGTCGCC |
| H7 | | GCAATCTGGCCGGGAGGGGGTCTGACCGTGTATGCTGACAGCGTGAAGGGCAGA |
| | | TTCACCATCTCTAGGGACCACGCCAAGAACACCCTCTATTTGCAGATGAACAAT |
| | | CTCAAACCCGAGGATACCGCGATGTATTACTGCGCTGCGGGTAGCCCACGTATG |
| | | TGTCCATCTCTGGAGTTCGGCTTCGATTATTGGGGTCAGGGCACTCAGGTGACC |
| | | GTCAGCTCCGGGGGCTCCGGCGGTTCTGGTGGGTCAGGCCAGGTGCAGCTCCAG |
| | | GAGTCCGGCGGTGGCCTGGTGCAGCCCGGTGGGTCCCTTCGTCTGAGCTGTGCA |
| | | GCCAGCGGATTTACATTTAGCTCCTACCCCATGTCCCGGGTAAGACAGGCCCCC |
| | | GGAAAGGGCCTGGAATGGATTTCCACCATCTCTGCCGGTGGAGACACCACACTG |
| | | TATGCTGACTCTGTGAAGGGAAGATTCACTTCCTCTCGGGACAACGCCAAGAAC |
| | | ACCCTGTACTTGCAGTTGAACAGCCCGAAGACAGAGGATACCGCCATTTATTAC |
| | | TGTGCCAAGAGGATTGATTGTAACAGCGGATACTGTTATCGCAGGAACTATTGG |
| | | GGACAGGGAACTCAGGTGACCGTCTCTTCA |
| DR591- | 969 | CAGGTCCAGCTTCAGGAGTCCGGCGGAGGCTCTGTGCAGGCAGGCGGTTCACTG |
| hIL27 | | CGCCTGTCATGCACGGCGAGCGGAGCCATCGCCAGCGGTTACATTGATTCCCGT |
| Ra_VH | | TGGTGCATGGCGTGGTTCCGTCAGGCACCCGGCAAGGAACGCGAGGGCGTGGCT |
| H8 | | GCCATCTGGCCTGGGGGGGGGCTTACAGTGTACGCCGATTCCGTGAAAGGTCGC |
| | | TTCACCATCTCCCGCGACCACGCCAAGAATACTCTTTACCTGCAAATGAATAAC |
| | | TTGAAGCCGGAGGACACCGCTATGTATTACTGTGCCGCTGGCAGCCCTCGCATG |
| | | TGTCCCTCTCTGGAGTTCGGCTTTGATTACTGGGGCCAGGGCACCCAGGTGACC |
| | | GTATCTAGCGGTGGAGGCTCCCAAGTGCAGCTGCAAGAATCTGGAGGGGGGAGC |
| | | GTCCAGGGGGAGGCTCCCCGCGCCTGAGCTGCGCCGCCTTCCGGCTTTACCTTT |
| | | AGTAGCTACCCCATGTCCTGGGTCCGCCAGGCACCCGGCAAAGGCCTTGAGTGG |
| | | ATCTCCACGATCAGCGCAGGGGGTGACACCACTCTGTACGCCGACAGCGTTAAA |
| | | GGCAGGTTCACCAGCTCCCGCGACAACGCGAAAAACACCCTGTACTTGCAGCTC |
| | | AACTCACTCAAGACGGAGGACACTGCTATCTATTACTGCGCCAAGCGCATTGAT |
| | | TGTAACAGCGGTTACTGTTACCGGCGTAACTACTGGGGTCAGGGCACTCAGGTC |
| | | ACCGTCTCCTCT |
| DR591- | 970 | CAAGTGCAGGTCCAGGAATCCGGTGGAGGCAGCGTGCAGGCCGGAGGTTCCCTG |
| hIL27 | | CGCCTGTCTTGCACTGCCTCAGGAGCCATTGCCTCCGGTTACATTGATTCCCGT |
| Ra_VH | | TGGTGCATGGCCTGGTTTCGCCAGGCCCCAGGAAAAGAGCGCGAGGGGGTGGCC |
| H8 | | GCTATCTGGCCCGGGGGTGGCCTGACTGTGTACGCTGATTCCGTAAAGGGCCGG |
| | | TTTACCATCTCCAGGGATCATGCAAAAAACACACTCTACCTCCAGATGAATAAC |
| | | CTGAAGCCTGAGGACACCGCCATGTATTACTGCGCCGCTGGCTCACCTCGTATG |
| | | TGTCCCAGCCTCGAATTTGGCTTTGACTATTGGGGCCAGGGCACCCAGGTTACC |
| | | GTCTCTTCCGGTGGCTCAGGAGGTTCCGGGGGCTCTGGTCAGGTGCAGTTGCAG |
| | | GAAAGTGGAGGTGGCAGTGTGCAGGTAGGGGGCAGCCTGCGTCTGAGCTGTGCT |
| | | GCCAGCGGCTTTACATTCAGCTCCTATCCTATGAGCTGGGTGAGACAGGCCCCC |
| | | GGGAAAGGCCTGGAGTGGATCAGCACTATCAGCGCCGGTGGCGATACGACTCTC |
| | | TATGCCGATAGCGTCAAGGGCCGCTTCACGTCCTCAAGGGACAATGCTAAAAAG |
| | | ACCTTGTATGTCCAGTTGAACAGTCTCAAGACGGAGGACACCGCTATCTACTAT |
| | | TGTGCCAAGAGAATTGATTGCAACAGGGGCTACTGTTACAGACGTAATTATTGG |
| | | GGGCAGGGCACCCAGGTAACTGTGTCCTCA |
| DR591- | 971 | CAGGTGCAGTTGCAGGAGTCAGGCGGTGGCTCCGTGCAGGCAGGCGGAAGCCTG |
| hIL27 | | CGCCTGTCTTGCACTGCCTCTGGGGCTATTGCATCAGGGTACATTGATAGCCGC |
| Ra_VH | | TGGTGTATGGCGTGGTTTAGGCAGGCTCCAGGAAAGGAAAGAGAGGGAGTGGCC |
| H9 | | GCGATCTGGCCCGGTGGAGGTCTGACGGTGTATGCTGATTCAGTGAAGGGCCGG |
| | | TTCACTATTTCTCGCGACCATGCCAAGAACACCCTCTACCTTCAGATGAATAAC |
| | | CTGAAACCCGAGGACACCGCTATGTATTACTGCGCTGGGGGTTCTCCCCGCATG |
| | | TGTCCTAGTCTTGAGTTCGGATTTGACTATTGGGGCCAGGGCACCCAAGTGACG |
| | | GTCAGCTCCGGGGGGGGTAGTCAGGTCCAACTGCAAGAATCCGGGGGGGGTAGC |
| | | GTGCAGTCCGGTGGCTCCCTGAGACTCAGCTGCGCCGGTTCCGGGTTTACCTAC |
| | | TCCACTTCCAACTCTTGGATGGCGTGGTTCCGTCAGGCCCCAGGAAAGGAGAGG |
| | | GAGGGCGTGGCCGCTATCTATACGGTGGGTGGGTCCATCTTCTACGCCGATTCT |
| | | GTACGCGGAAGATTCACAATCTCTCAGGACGCAACAAAAAATATGTTTTACTTG |
| | | CAGATGAACACTCCGAAGCCTGAAGACACTGCGATGTACTATTGTGCGGCTGCG |
| | | AGCGGCAGACTGCGCGGTAAGTGGTTCTGGCCCTACGAGTACAACTACTGGGGG |
| | | CAGGGCACTCAGGTCACAGTCTCTAGC |
| DR591- | 972 | CAGGTGCAGTTGCAGGAATCTGGCGGTGGCAGTGTCCAGGCCGGTGGCTCCCTC |
| hIL27 | | AGGCTGTCCTGCACTGCCAGGGGAGCCATCGCTTCAGGCTACATTGACTCTAGG |
| Ra_VH | | TGGTGCATGGCTTGGTTTCGCCAGGCTCCGGGCAAAGAGCGCGAGGGCGTCGCA |
| H9 | | GCCATCTGGCCAGGTGGGGGTTTGACGGTCTATGCTGATTCCGTGAAGGGGCGG |
| | | TTCACTATCTCCCGTGACCACGCCAAGAACACCCTGTACCTCCAGATGAATAAC |
| | | CTGAAGCCTGAGGATACAGCGATGTACTATTGCGCTGCCGGTAGCCCGCGCATG |
| | | TGCCCCAGTCTTGAGTTCGGCTTTGACTATTGGGGCCAGGGCACGCAAGTGACC |
| | | GTGTCCTCTGGGGGCAGTGGCGGTTCTGGTGGCTCAGGTCAGGTCCAGCTCCAG |
| | | GAAAGCGGGGGGGGTCCGTCCAATCTGGTGGGTCCTTGCGTCCTGAGCTGCGCT |
| | | GCCTCTGGCTTCACTTATTCAACGTCCAACTCTTGGATGGCCTGGTTTCGCCAA |
| | | GCGCCAGGCAAGGAGCGTGAAGGCGTCGCTGCCATCTATACCGTTGGTGGCAGC |
| | | ATTTTCTACGCTGACTCAGTGAGGGGCAGATTTACCATTTCTCAGGATGCAACC |
| | | AAGAATATGTTTTACTTGCAGATGAACACACTGAAGCCCGAGGACACAGCTATG |
| | | TACTATTGTGCAGCCGCTTCTGGCCGTCTTCGCGGCAAGTGGTTCTGGCCCTAC |
| | | GAGTACAATTATTGGGGCCAGGGTACGCAGGTGACCGTGTCCAGT |
| DR591- | 973 | CAAGTTCAGTTGCAGGAGTCCGGTGGCGGTAGTGTGCAGGGGGGGGGAAGCCTG |
| hIL27 | | CGCCTGTCTTGTACCGCCTCTGGGGCCATTGCCTCTGGCTACATTGATTCTCGC |
| Ra_VH | | TGGTGTATGGCCTGGTTTAGGCAGGCACCAGGCAAGGAACGCGAAGGGGTGGCC |
| H10 | | GCTATCTGGCCTGGGGGCGGACTGACCGTGTACGCTGACTCAGTCAAAGGCCGC |
| | | TTCACTATCTCACGCGACCACGCCAAGAATACTCTGTACCTCCAGATGAATAAC |
| | | CTGAAGCCTGAGGACACCGCGATGTATTACTGCGCTGCCGGTTCCCCAAGAATG |
| | | TGCCCTTCACTTGAGTTTGGCTTTGACTATTGGGGCCAAGGAACTCAGGTGACC |
| | | GTAAGCTCTGGAGGGGGCAGCCAGGTCCAGCTCCAGGAGTCTGGAGGGGGCTCC |
| | | GTGCAGGCCGGAGGGTCTCTCCGCCTCAGCTGTCGTGCCTCAGGGTCCACGTAC |
| | | TCTAACTACTGCCCGGGGGGTTCCGCCAGATCACAGGTAAAGAGCCGCGAAGGC |
| | | GTAGCTGTCATCAACTGGGTCGGGGGAATGCTGTACTTCGCCGACTCCGTTAAG |
| | | GGTCGGTTCACCGTCTCTCAGGATCAGGCCAAAAATACCCTGTATCTCCAGATG |
| | | AACAGCCTGAAGCCGGAGGACACCGCTATGTATTACTGTGCCGCAGAGAGCGTG |
| | | TCCTCTTTCTCCTGCGGCGGTTGGCTGACCCGCCCGGACAGAGTACCCTACTGG |
| | | GGTCAGGGGACTCAGGTGACAGTGAGCAGC |
| DR591- | 974 | CAGGTGCAACTGCAAGAGTCCGGCGGAGGCTCTGTGCAAGCCGGAGGCTCTCTG |
| hIL27 | | CGCCTGTCCTGTACCGCTAGTGGAGCCATCGCCAGCGGTTACATTGACAGCAGA |
| Ra_VH | | TGGTGTATGGCCTGGTTCCGCCAGGCCCCCGGCAAAGAGCGCGAGGGCGTCGCT |
| H10 | | GCAATCTGGCCTGGGGGGGGCCTTACCGTATATGCCGATTCAGTCAAAGGCCGC |
| | | TTCACAATCTCACGCGACCACGCCAAGAACACCCTGTACTTGCAGATGAATAAC |
| | | CTGAAGCCGGAGGACACCGCAATGTATTACCGCGCAGCCGGGTCTCCGCGCATG |
| | | TGTCCCAGCTTGGAGTTCGGATTCGACTACTGGGGCCAAGGGACCCAGGTGACG |
| | | GTTAGCTCTGGCGGTTCCGGCGGGAGTGGGGGCTCTGGCCAGGTGCAGCTGCAA |
| | | GAGTCTGGGGGAGGTTCTGTCCAGGGGGGAGGCTCCCTGAGGCTGAGTTGTCGT |
| | | GCAAGCGGTTCTACCTACTCCAACTATTGCCTGGGTTGGTTTCGTCAGATTACG |
| | | GGGAAGGAACGCGAGGGGGTCGCCGTCATAAATTGGGTTGGCGGTATGCTTTAT |
| | | TTTGCCGACTCTGTCAAAGGTCGGTTCACCGTGTCTCAAGACCAGGCTAAGAAC |
| | | ACTCTGTACGTCCAGATGAACTCCCTGAAACCAGAGGACACCGCAATGTATTAC |
| | | TGCGCTGCCGAAAGCGTGTCCAGCTTCAGCTGTGGCGGTTGGCTCACCCGCCCT |
| | | GACCGTGTGCCCTATTGGGGTCAGGGGACACAGGTGACCGTCAGCAGC |
| DR591- | 975 | CAGGTGCAGTTGCAGGAAAGCGGAGGGGGCTCCGTGCAAGCCGGAGGGTCTCTG |
| hIL27 | | CGTCTTTCTTGCACTGCCTCTGGGGCCATCGCTTCCGGGTACATTGATAGCCGT |
| Ra_VH | | TGGTGCATGGCCTGGTTCCGTCAAGCGCCTGGAAAGGAGCGCGAGGGCGTGGCC |
| H11 | | GCTATTTGGCCTGGTGGAGGCCTCACTGTGTATGCTGATAGTGTGAAGGGACGT |
| | | TTCACAATCTCCCGTGACCACGCGAAGAACACGCTGTACCTCCAGATGAACAAT |
| | | CTCAAACCCGAGGATACAGCTATGTATTACTGTGCCGCTGGTAGTCCACGCATG |
| | | TGTCCCAGTCTGGAGTTCGGGTTTGATTATTGGGGACAAGGCACGCAGGTTACA |
| | | GTGTCTTCCGGGGGGGCTCCCAAGTTCAGCTTCAAGAGAGCCGGCGGAGGCTCC |
| | | GTGCAGGCGGGTGGCTCACTGAGACTGTCCTGCCGTGCCTCCGGGTCCACTTAT |
| | | AGTAACTATTGTCTGGGTTGGTTCCGTCAAAGTACTGGTAAAGAGCGCGAGGGG |
| | | GTGGCAGTAATCAACTGGGTCGGCGGAATGCTGTACTTCGCGGATTCTGTGAAG |
| | | GGAAGATTCACTGTGTCACAGGACCACGCCAAAAATACCGTGACCCTTCAGATG |
| | | AATAGCCTGAAGCCTGAGGACACGGCAATGTATTACTGTGCCGCTGAATCCGTG |
| | | TCTAGCTTTAGCTGCGGAGGTTGGCTGACACGTCCTGGTAGAGTGCCATATTGG |
| | | GGCCAAGGCACTCAAGTGACTGTATCTTCC |
| DR591- | 976 | CAGGTTCAGCTTCAGGAATCCGGTGGAGGCAGTGTGCAGGCCGGTGGAAGCCTC |
| hIL27 | | CGCCTCTCTTGCACAGCCTCCGGGGCCATCGCATCTGGCTATATTGACAGCCGC |
| Ra_VH | | TGGTGTATGGCTTGGTTCCGCCAAGCGCCCGGTAAGGAGCGCGAAGGGGTCGCT |
| H11 | | GCCATTTGGCCTGGTGGGGGATTGACTGTGTATGCAGATAGCGTGAAAGGTCGT |
| | | TTCACTATCAGCCGTGACCACGCCAAGAACACCCTGTATCTGCAAATGAACAAT |
| | | CTGAAGCCTGAGGACACCGCCATGTATTACTGTGCGGCAGGCAGCCCTCGCATG |
| | | TGCCCGTCCCTGGAGTTCGGTTTCGACTACTGGGGCCAGGGCACACAGGTGACT |
| | | GTTAGCTCCGGCGGTTCTGGAGGTTCTGGGGGCTCTGGCCAGGTACAGCTCCAG |
| | | GAGAGCGGAGGGGGCTCCGTTCAAGCAGGTGGCTCTTTGCGTTTGAGCTGCCGT |
| | | GCCAGCGGGTCTACCTACTCCAATTACTGTCTGGGATGGTTCCGCCAATCCACC |
| | | GGCAAAGAACGTGAGGGTGTGGCCGTTATCAACTGGGTTGGGGGAATGCTTTAC |
| | | TTTGCGGACAGTGTGAAAGGGCGCTTCACCGTGTCCCAGGATCATGCTAAGAAC |
| | | ACTGTGACCCTCCAGATGAACAGCCTGAAGCCCGAGGATACGGCAATGTATTAC |
| | | TGCGCCGCTGAATCAGTAAGCTCCTTCTCATGTGGGGGATGGCTGACCCGCCCT |
| | | GGCCGCGTGCCCTATTGGGGACAGGGCACCCAGGTGACAGTCAGCAGC |
| DR591- | 977 | CAGGTGCAGTTGCAGGAATCCGGTGGGGGAAGTGTGCAGGCCGGGGGCTCCCTC |
| hIL27 | | CGTCTGTCCTGCACCGCGTCAGGCGCTATCGCCTCCGGGTACATCGACTCCCGC |
| Ra_VH | | TGGTGCATGGCCTGGTTTCGTCAAGCGCCCGGCAAGGAGAGAGAAGGTGTGGCC |
| H12 | | GCGATTTGGCCGGGCGGAGGCGTGACCGTGTACGCTGACTCCGTTAAGGGGCGT |
| | | TTTACTATCTCCCGTGACCACGCCAAGAATACATTGTATCTGCAAATGAATAAC |
| | | CTCAAGCCCGAGGACACTGCTATGTACTATTGTGCTGCCGGTTCCCCGCGCATG |
| | | TGTCCGAGCCTGGAGTTTGGCTTCGACTATTGGGGTCAAGGCACTCAGGTCACC |
| | | GTCTCCTCAGGCGGAGGGAGCCAAGTGCAGCTCCAGGAAAGCGGAGGTGGCTCC |
| | | GTGCAGGCTGGAGAGAGCCTGAGGCTGTCATGCCGCGCCAGGGGGTCTACCTAC |
| | | TCTAACTACTGTCTGGGCTGGTTTAGGCAGATTACCGGCAAAGAGAGAGAGGGG |
| | | GTCGCTGTTATCAACTGGGTGGGAGGGATGTTGTACTTCGCCGACAGCGTCAAG |
| | | GGTAGATTTACCGTCTCTCAGGACCAGGCTAAGAACACAGTATATCTGGAGATG |
| | | AACTCTCTTAAACCCGAGGACACTGCTATGTACTATTGTGCCACCGAGTCCGTG |
| | | TCTTCCTTCTCCTGCGGGGGCTGGCTGACTAGACCTGATCGGGTGCCCTACTGG |
| | | GGTCAGGGTACACAGGTGACAGTCTCTAGC |
| DR591- | 978 | CAAGTTCAGGTTCAGGAAAGTGGCGGTGGCTCGGTACAGGCCGGGGGCAGTCTG |
| hIL27 | | CGCCTGAGCTGCACCGCCAGCGGTGCCATCGCCAGGGGCTACATCGACTCCAGG |
| Ra_VH | | TGGTGTATGGCTTGGTTCCGCCAAGCACCGGGCAAGGAGCGCGAAGGAGTTGCC |
| H12 | | GCTATCTGGCCCGGGGGGGGCCTGACCGTCTACGCCGACAGCGTGAAAGGTCGC |
| | | TTCACTATCTCTCGCGACCACGCCAAGAACACCTTGTACCTCCAAATGAATAAC |
| | | CTGAAGCCAGAGGACACTGCTATGTATTACCGCGCTGCCGGAAGTCCCCGTATG |
| | | TGTCCCTCTGGGAGTTTGGGTTTGATTATTGGGGGCAGGGCCACACAGGTCACT |
| | | GTGTCAAGCGGGGGTAGTGGCGGTTCCGGCGGTAGCGGCCAGGTGCAGCTTCAA |
| | | GAGAGCGGGGGCGGAAGCGTGCAGGCGGGCGAATCTCTCCGGTTGTCATGTAGA |
| | | GCGTCTGGCTCCACCTACTCTAACTACTGCCTGGGGGGTTCAGACCAGATTACG |
| | | GGGAAAGAGAGGGAAGGCGTGGCCGTTATAAATTGGGTAGGCGGAATGCTCTAG |
| | | TTCGCTGACTCTGTCAAGGGCCGGTTCACAGTGTCTCAGGACCAGGCCAAAAAT |
| | | ACTGTGTATGTGGAGATGAACTCTCTGAAACCCGAGGACACTGCCATGTATTAC |
| | | TGCGCCACGGAGAGCGTCTCCAGCCTTCTCATGTGGAGGGGGCTGACCCGCCCG |
| | | GACCGTGTACCCTACTGGGGTCAGGGAACACAGGTGACAGTGAGCAGC |
| DR591- | 979 | CAGGTGCAGTTGCAGGAGAGCGGAGGGGGGAGCGTCCAGGCCGGAGGCTCCCTT |
| hIL27 | | CGCCTCAGCTGTACCGCGTCTGGAGCAATCGCCAGCGGGTACATTGACTCCCGT |
| Ra_VH | | TGGTGCATGGCATGGTTCCGCCAAGCGCCAGGCAAAGAGAGAGAAGGCGTGGCC |
| H13 | | GCTATTTGGCCTGGGGGTGGACTCACAGTTTATGCAGACTCTGTGAAGGGCCGC |
| | | TTTACCATCAGTAGAGACCACGCCAAGAACACCCTGTATCTGCAAATGAACAAT |
| | | CTGAAGCCAGAGGACACCGCGATGTACTATTGCGCTGCCGGTTCTCCCAGGATG |
| | | TGTCCCTCATTGGAGTTCGGCTTTGATTACTGGGGCCAGGGAACCCAGGTCACC |
| | | GTATGTTCCGGTGGGGGATCACAGGTTCAGCTCCAGGAATCAGGAGGTGGCTCT |
| | | GTGCAGGCCGGGGGCTCCCTGCGGCTGTCTTGCGTGGCCTCCGGCTACGTCTCT |
| | | TGCGACTACTTTCTGCCTTCTTGGTATCGGCAGGCCCCTGGGAAAGAGCGGGAG |
| | | TTCGTCTCTATCATTGACGGCACCGGCTCCACATCCTACGCTGCGAGCGTGAAG |
| | | GGCCGCTTCACAGCATCCCAGGACAGGGGAAAGAACATCGCTTATTTGCAGATG |
| | | AACAGCCTGAAGCCTGAGGACACAGCTATGTATTACTGTAAGGCCTCTTGTGTG |
| | | CGCGGCAGAACCATCAGCGAGTACTGGGGACAAGGGACCCAGGTGACCGTATCT |
| | | TCA |
| DR591- | 980 | CAGGTGCAGTTGCAGGAGTCCGGGGGGGGCTCTGTCCAAGCTGGAGGCTCCTTG |
| hIL27 | | CGTTTGAGCTGTACCGCTAGTGGAGCCATCGCGAGTGGCTACATCGACAGCAGA |
| Ra_VH | | TGGTGCATGGCCTGGTTCCGCCAAGCTCCCGGCAAGGAGAGGGAAGGCGTTGCT |
| H13 | | GCAATCTGGCCCGGCGGTGGCGTGACCGTGTACGCGGATTCTGTCAAGGGGCGC |
| | | TTTACTATCAGCCGCGATCATGCTAAAAACACGCTTTATCTGCAAATGAATAAC |
| | | CTCAAACCAGAAGATACCGCGATGTACTATTGCGCAGCTGGTTCTCCCCGGATG |
| | | TGTCCCAGTTTGGAGTTTGGTTTCGATTACTGGGGTCAGGGCACTCAGGTGACC |
| | | GTGAGTTCCGGCGGATCTGGAGGCTCAGGAGGCTCCGGCCAGGTGCAGCTCCAG |
| | | GAGTCCGGCGGTGGCAGCGTACAAGCTGGCGGGTCCCTCCGTCTGAGCTGTGTG |
| | | GCAAGCGGTTATGTTAGCTGTGACTACTTTCTGCCAAGCTGGTATCGCCAAGCG |
| | | CCAGGCAAGGAGCGCGAGTTCGTGAGTATCATTGATGGCACCGGCAGCACCTCC |
| | | TACGCAGCCAGCGTGAAGGGACGGTTTACCGCCTGCCAGGATAGAGGAAAGAAC |
| | | ATCGCATACCTCCAGATGAACAGCCTGAAGCCGGAGGACACAGCCATGTATTAC |
| | | TGCAAGGCCTCTTGCGTGAGGGGCCGCACCATCAGCGAGTACTGGGGGCAGGGC |
| | | ACTCAGGTAACTGTCAGCTCA |
| DR591- | 981 | CAGGTGCAGCTTCAGGAGAGCGGTGGAGGCTCTGTCCAAGCTGGCGGTTCCCTG |
| hIL27 | | AGACTTTCCTGCACCGCCTCCGGGGCCATCGCTTCCGGCTACATTGATAGCAGA |
| Ra_VH | | TGGTGTATGGCATGGTTCAGGCAGGCCCCTGGTAAAGAGCGCGAGGGGGTTGCC |
| H14 | | GCAATCTGGCCTGGGGGAGGCCTGACCGTGTACGCCGACTCCGTGAAGGGCCGT |
| | | TTCACCATCTCTCGTGACCACGCCAAGAATACTCTGTATCTTCAGATGAATAAC |
| | | TTGAAACCTGAAGACACAGCTATGTATTACCGCGCAGCCGGAAGCCCACGCATG |
| | | TGCCCATCCCTGGAGTTTGGCTTCGATTATTGGGGCCAAGGCACCCAAGTGACA |
| | | GTCAGCAGTGGAGGCGGTTCCCAGGTTCAGTTGCAAGAGTCCGGTGGCGGAAGC |
| | | GTGCAGGCAGGCGGTAGCTTGGGCTTGTCCTGTGTGGCCTCCGGGTATGTGAGT |
| | | TGCGACTATTTCCTGCCTTCCTGGTATAGACAGGCCCCCGGCAAGGAACGCGAG |
| | | TTCGTGTCTATTATCGACGGCACCGGGAGCACATCCTACGCTGCGAGCGTCAAG |
| | | GGCCGCTTCACTGCGTCACAGGACAAGGGCAAGAACATCGCTTATCTCCAGATG |
| | | AACTCTCTGAAACCTGAGGATACAGCAATGTATTACTGTAAGGCTTCCTGCGTG |
| | | AGAGGCCGCGCCATTAGCGAGTACTGGGGGCAGGGCACTCAGGTAACCGTAAGC |
| | | AGC |
| DR591- | 982 | CAGGTTCAGGTTCAGGAGTCTGGGGGAGGCAGGGTGCAGGCAGGTGGCTCCCTT |
| hIL27 | | CGCCTCAGCTGTACGGCTTCAGGTGCCATTGGTTCCGGTTACATTGATAGCAGG |
| Ra_VH | | TGGTGTATGGCCTGGTTCCGGCAAGCGCCCGGCAAAGAAAGAGAGGGTGTGGCA |
| H14 | | GCCATCTGGCCAGGCGGTGGGCTTACCGTGTATGCCGATTCTGTTAAGGGCAGG |
| | | TTCACGATCTCCAGGGACCACGCCAAGAATACCCTGTATCTCCAAATGAATAAC |
| | | CTGAAGCCCGAGGACACTGCCATGTATTACTGTGCTGCGGGCTCCCCTCGTATG |
| | | TGTCCCTCTCTGGAGTTCGGGTTTGACTACTGGGGACAAGGAACACAGGTGACC |
| | | GTCTCCAGCGGGGGCAGCGGAGGTAGCGGCGGAAGCGGACAGGTACAGTTGCAG |
| | | GAGTCTGGCGGTGGCTCCGTGCAGGCTGGCGGTTCACTCAGACTGTCCTGTGTG |
| | | GCCAGCGGATACGTGAGCTGCGATTATTTCTTGCCTTCCTGGTATCGCCAGGCC |
| | | CCTGGGAAGGAACGCGAGTTCGTGTCTATCATTGACGGAACCGGCTCCACGTCC |
| | | TATGCCGCTTCTGTCAAGGGTCGCTTTACAGCTTCCCAGGACAAGGGTAAAAAC |
| | | ATCGCTTACGTTCAGATGAACTCCCCGAAGCCTGAAGATACCGCCATGTACTAT |
| | | TGTAAGGCCAGCTGCGTGCGGGGCAGGGCTATCTCAGAGTATTGGGGTCAGGGC |
| | | ACGCAGGTGACAGTGTCCTCT |
| DR591- | 983 | CAGGTGCAGCTCCAGGAAAGCGGGGGAGGCCTCCGTGCAGGGGGGGGTAGCCTC |
| hIL27 | | CGTCTGTCCTGCACTGCCTCAGGAGCCATTGCCTCTGGGTACATTGACTCCCGC |
| Ra_VH | | TGGTGTATGGCCTGGTTCCGCCAAGCGCCCGGAAAGGAAAGGGAGGGCGTGGCT |
| H15 | | GCAATTTGGCCAGGAGGTGGCCTGACCGTGTATGCGGATTCTGTGAAGGGACGC |
| | | TTCACCATCAGCAGGGACCACGCTAAGAACACCGTGTACCTGCAAATGAATAAC |
| | | CTGAAACCCGAGGACACCGCCATGTACTATTGTGCCGCAGGCTCCCCCAGAATG |
| | | TGCCCTTCTCTGGAGTTCGGGTTCGACTATTGGGGTCAGGGGACCCAGGTGACC |
| | | GTCTCCTCTGGTGGGGGTAGCCAGGTGCAACTCCAGGAATCCGGGGGGGGCTCC |
| | | GTGCAGGCTGGCGGGTCTCTGCGCCTGAGCTGTGTGGCTTCCGGGTATGTGTCC |
| | | TGCGACTACTTCCTGCCTTCTTGGTATCGCCAAGCGCCGGGGAAGGAACGCGAG |
| | | TTCGTAAGCATCATTGATGGCACTGGATCAACAAGCTACGCGGCGTCTGTGAAA |
| | | GGCCGCTTCACAGCGAGCCAGGATAAGGGTAAGAACATCGCGTATTTGCAGATG |
| | | AACACTCTCAAGCCAGAAGACACCGCGATGTATTACTGCAAGGCCAGTTGTGTG |
| | | CGTGGCCGGGCCATCTCCGAGTATTGGGGGCAGGGCACCCAGGTCACCGTGTCC |
| | | TCC |
| DR591- | 984 | CAGGTCCAACTTCAGGAGTCCTGGAGGCGGTTCCGTGCAGGGGGGGGTAGCTTG |
| hIL27 | | CGCCTGAGCTGTACCGCTTCAGGTGCCATCGCGAGCGGATACATCGACTCTCGC |
| Ra_VH | | TGGTGTATGGCCTGGTTCCGCCAGGCCCCTGGCAAGGAACGTGAGGGTGTGGCT |
| H15 | | GCCATTTGGCCCGGCGGGGGACTTACTGTGTACGCCGACTCCGTGAAGGGCCGT |
| | | TTCACCATCTCCCGTGACCACGCGAAGAACACCCTGTACTTGCAGATGAATAAC |
| | | CTCAAACCCGAGGATACCGCGATGTATTACTGTGCCGCTGGCAGCCCACGCATG |
| | | TGTCCCAGTCTGGAGTTCGGTTTTGACTATTGGGGGCAGGGTACACAAGTCACA |
| | | GTGTCTAGCGGAGGCTCTGGTGGCTCCGGGGGTAGCGGACAGGTGCAGTTGCAG |
| | | GAATCTGGCGGTGGCAGTGTGCAGGCGGGTGGCAGCCTGCGCCTGTCCTGCGTT |
| | | GCTAGTGGGTATGTGAGCTGTGATTATTTCCTGCCATCTTGGTATCGCCAGGCC |
| | | CCCGGCAAAGAAAGAGAGTTCGTGTCCATCATTGATGGCACCGGCTCCACCAGT |
| | | TATGCAGCGTCTGTCAAAGGCAGGTTCACTGCCAGCCAGGACAAGGGTAAGAAC |
| | | ATTGGTTATTTGCAGATGAACACCCTGAAGCCCGAGGACACGGCTATGTATTAC |
| | | TGTAAGGCCTCATGTGTGCGTGGCCGGGCGATTTCCGAGTACTGGGGACAAGGG |
| | | ACACAAGTGACCGTGTCCAGC |
| DR591- | 985 | CAAGTGCAACCTCCAGGAGTCCGGGGGGGGTCTGTGCAAGCTGGAGGCTCCCTG |
| hIL27 | | CGCCTGAGTTGTACCGCGTCCGGTGCCATCGGTTCTGGTTATATTGATAGCAGA |
| Ra_VH | | TGGTGTATGGCTTGGTTCCGTCAAGCGCCAGGGAAAGAGAGGGAGGGCGTGGCA |
| H16 | | GCTATTTGGCCAGGAGGCGGACTGACCGTCTACGCAGACTCCGTCAAAGGCCGG |
| | | TTTACAATCTCCAGAGACCACGCGAAAAACACTCTGTATCTCCAGATGAATAAC |
| | | CTGAAGCCAGAGGATACAGCAATGTACTATTGCGCCGCTGGCTCACCCCGTATG |
| | | TGCCCCAGTCTGGAGTTCGGGTTTGACTACTGGGGCCAGGGCACCCAAGTCACC |
| | | GTAAGCTCCGGCGGTGGCTCCCAGGTGCAGTTGCAGGAAAGTGGGGGAGGCAGC |
| | | GTGCAGGCCGGGGGCAGCCTGCGCCTCTCTTGTCGCGCATCCGGCAGTACCTAT |
| | | TCTAACTACTGTTTGGGTTGGTTTAGGCAGATTACCGGCAAGGAGAGGGAGGGG |
| | | GTGGCCGTTATCAACTGGGTCGGGGGCATGTTGTACTTTGCCGACTCTGTGAAG |
| | | GGTAGGTTCACTGTTTCTCAGGATCAGGCTAAGAACACTGTTTATCTTCAGATG |
| | | AACTCTCTGAAGCCAGAGGACACCGCTATGTATTACTGTGCCGCAGAGTCAGCC |
| | | AGCTCCTTTAGTTGTGGGGGCTGGCTGACCCGTCCCGACCGCGTGCCATACTGG |
| | | GGCCAGGGTACACAAGTGACTGTCTCCAGC |
| DR591- | 986 | CAGGTGCAACTTCAGGAGAGTGGGGGAGGCTCTGTTCAGGCAGGGGGGTCCCTG |
| hIL27 | | CGCCTGAGCTGCACCGCCTCAGGTGCCATCGCCTCTGGCTATATTGATAGCCGT |
| Ra_VH | | TGGTGTATGGCCTGGTTCCGGCAGGCACCGGGCAAAGAACGCGAAGGGGTGGCC |
| H16 | | GCTATCTGGCCTGGAGGTGGCCTGACCGTTTACGCTGACAGCGTGAAAGGAAGG |
| | | TTCACCATCTCCAGGGACCACGCTAAGAACACTCTGTACTTGCAGATGAATAAC |
| | | CTGAAACCTGAAGATACCGCCATGTATTACTGCGCCGCTGGCTCTCCACGCATG |
| | | TGTCCATCCCTGGAGTTCGGATTCGATTATTGGGGACAGGGCACCCAGGTCACC |
| | | GTGTCCTCTGGCGGATCTGGTGGCTCAGGGGGCTCTGGCCAGGTCCAGTTGCAG |
| | | GAATCCGGTGGAGGCAGTGTTCAGGCCGGGGGCTCCCTGCGCCTGTCATGTCGG |
| | | GCCTCCGGCAGCACCTACTCCAATTACTGCCTCGGATGGTTCCGCCAGATCACC |
| | | GGCAAAGAGCCGTGAAGGCGTGGCCGTCATCAACTGGGGGGGGGGATGGTTTAC |
| | | TTTGCAGATTCCGTGAAGGGCAGATTTACGGTGTCTCAGGACCAGGCGAAGAAC |
| | | ACAGTGTATCTCCAGATGAACAGCCTCAAGCCTGAAGATACCGCTATGTATTAC |
| | | TGCGCCGCAGAGAGCGCTTCTTCCTTTAGCTGCGGCGGTTGGCTGACCAGGCCC |
| | | GACCGTGTTCCGTACTGGGGTCAGGGCACCCAGGTGACCGTATCCAGT |
| DR591- | 987 | CAAGTCCAGCTCCAGGAATCCGGGGGAGGTAGCGTGCAGGCGGGTGGCAGCCTG |
| hIL27 | | CGCCTGTCCTGTACTGCAAGGGGAGCCATCGCCTCTGGCTACATCGACTCACGC |
| Ra_VH | | TGGTGTATGGCCTGGTTTCGCCAAGCGCCGGGTAAGGAGCGCGAAGGGGTGGCC |
| H17 | | GCGATTTGGCCGGGGGGGGGCCTGACCGTCTACGCTGACAGTGTCAAGGGCCGC |
| | | TTCACCATCTCTCGGGATCACGCCAAGAACACTTTGTATCTGCAAATGAATAAC |
| | | CTGAAACCCGAAGACACCGCAATGTATTACTGCGCCGCTGGTAGTCCGCGTATG |
| | | TGTCCATCACTGGAGTTCGGCTTCGACTACTGGGGTCAGGGAACCCAGGTCACC |
| | | GTGTCCTCTGGTGGCGGTAGCCAGGTCCAGCTGCAAGAATCCGGTGGGGGGCTG |
| | | GTGCAGCCAGGTGGATCTCTGCGCTTGTCCTGTGCCGCAAGCGGTTTTACTTTC |
| | | TCACTGAGCGGCATGTCCTGGGTGAGGCAGGCTCCAGGCAAGGGCCTCGAATGG |
| | | GTCTCAGCCATTAGTAGCGGGGGCGCATCCACCTATTACACAGATTCCGTGAAG |
| | | GGTAGATTCACGATTAGCCGCGACAATGCTAAGAATATGCTGTATCTTCAGTTG |
| | | AATAGCCTGAAGACAGAGGACACCGCTATGTATTACTGCGCGAAGGGGGGATCT |
| | | GGCTACGGCGACGCCTCCAGGATGACAAGTCCGGGTTCCCAGGGCACACAGGTC |
| | | ACGGTCTCTAGC |
| DR591- | 988 | CAGGTGCAGCTCCAGGAGTCCGGGGGAGGCTCAGTCCAGGCTGGGGGCCCCCTC |
| hIL27 | | CGCCTGTCCTGTACGGCAAGTGGCGCTATCGCGAGCGGGTACATTGATTCACGC |
| Ra_VH | | TGGTGCATGGCCTGGTTCAGGCAAGCTCCTGGGAAGGAGCGCGAGGGAGTGGCT |
| H17 | | GCCATCTGGCCAGGGGGTGGCCTGACTGTGTACGCGGACAGCGTGAAGGGTCGC |
| | | TTCACAATCTCAAGGGACCACGCCAAGAATACGTTGTATCTTCAGATGAATAAC |
| | | CTGAAACCCGAGGACACAGCCATGTACTATTGTGCAGCCGGTTCCCCGAGAATG |
| | | TGCCCTAGTTTGGAGTTCGGCTTCGACTACTGGGGTCAGGGAACCCAGGTGACC |
| | | GTGTCCAGTGGGGGGTCTGGGGGTTCCGGCGGGTCAGGCCAGGTCCAGCTTCAG |
| | | GAATCCGGGGGAGGCCTGGTTCAGCCAGGTGGAAGCCTTCGCCTGTCCTGCGCT |
| | | GCATCCGGGTTCACATTCAGCCTGAGTGGCATGTCCTGGGTGAGACAGGCCCCA |
| | | GGAAAGGGTGTGGAGTGGGTCTCTGCAATCAGGTCCGGCGGTGCTTCTACCTAT |
| | | TACACCGACTCCGTGAAGGGCCGGTTCACTATTAGTCGCGACAACGCTAAGAAC |
| | | ATGCTGTACCTGCAACTGAACTCCTTGAAGACAGAAGACACGGCCATGTATTAC |
| | | TGCGCCAAGGGAGGTTCAGGTTACGGCGACGCCAGCCGCATGACCTCCCCTGGC |
| | | TCTCAGGGGACTCAGGTTACAGTCTCCTCC |
| DR591- | 989 | CAGGTCCAGCTCCAGGAAAGCGGAGGGGGGTCCGTCCAGGCTGGCGGTAGTTTG |
| hIL27 | | CGCCTCTCATGTACCGCCAGGGGCGCAATCGCTTCTGGATATATCGACTCTCGC |
| Ra_VH | | TGGTGTATGGCCTGGTTCCGGCAGGCCCCCGGCAAGGAGAGGGAGGGAGTGGCA |
| H18 | | GCCATCTGGCCGGGTGGCGGGTTGACCGTGTATGCCGACTCTGTCAAGGGCCGC |
| | | TTCACAATCAGCAGAGACCATGCCAAGAACACCCTGTACCTTCAGATGAATAAC |
| | | CTGAAGCCCGAAGACACCGCCATGTATTACCGCGCCGCAGGCTCCCCAAGAATG |
| | | TGCCCTAGTCTGGAGTTCGGCTTCGACTACTGGGGCCAGGGGACCCAGGTGACC |
| | | GTCAGCTCTGGTGGAGGTAGCCAGGTGCAACTCCAGGAGAGCGGAGGCGGTAGC |
| | | GTGCAGGCTGGCGGTTCCCTGCGCCTGAGCTGCGTGGCCTCTGGTTACGTTAGT |
| | | TGCGACTATTTTCTGCCCAGCTGGTATCGTCAGGCTCCGGGCAAGGAAAGAGAA |
| | | TTTGTCTCCATCATTGACGGAACTGGCTCTACAAGTTACGCCGCAAGTGTCAAG |
| | | GGTCGCTTCACCGCCAGCCAGGACAAGGGTAAGAACATCGCCTACCTGCAAATG |
| | | AACTCCCTCAAGCCAGAAGACACCGCGATGTACTATTGCAAGGCCAGCTGTGTA |
| | | AGGGGCAGGGGCATTAGCGAGTACTGGGGCCAGGGAACCCAGGTGACCGTGTCC |
| | | TCC |
| DR591- | 990 | CAGGTCCAGCTCCAGGAGTCAGGCGGTGGATCTGTGCAGGGGGGGGGGAGCCTC |
| hIL27 | | AGGCTGAGCTGTACTGCCAGCGGCGCTATTGCCTCCGGTTACATTGACAGCCGC |
| Ra_VH | | TGGTGTATGGCTTGGTTTCGCCAAGCCCCTGGAAAGGAGAGGGAGGGCGTTGCT |
| H18 | | GCCATCTGGCCTGGTGGGGGACTGACCGTCTATGCTGATTCAGTGAAGGGGCGT |
| | | TTTACCATCTCCCGCGATCACGCTAAGAACACACTGTACCTCCAGATGAACAAT |
| | | TTGAAGCCCGAAGACACAGCCATGTACTATTGCGCCGCTGGGAGTCCCCGCATG |
| | | TGCCCTAGCGTGGAGTTTGGCTTCGACTATTGGGGCCAGGGCACTCAGGTCACT |
| | | GTTAGTTCCGGTGGCTCTGGTGGCAGCGGTGGCAGTGGCCAAGTGCAGCTCCAG |
| | | GAGTCCGGCGGAGGCTCAGTACAGGCCGGTGGCTCCCTTCGCCTGTCCTGCGTC |
| | | GCCTCCGGTTACGTTAGTTGCGACTATTTCCTGCCCAGGTGGTATCGCCAGGCA |
| | | CCAGGGAAGGAGCGCGAGTTCGTGTCCATTATCGACGGAACAGGGTCTACATCC |
| | | TATGCTGCCAGCCGTGAAAGGAAGGTTCACCGCCCCCAGGATAAGGGAAAAAAC |
| | | ATCGCCTACGTCCAGATGAACAGCCTGAAGCCAGAGGACACGGCTATGTACTAT |
| | | TGCAAGGCCTCCTGCGTGCGCGGCAGAGGTATCTCCGAATATTGGGGCCAGGGA |
| | | ACACAGGTGACCGTGAGCAGC |
| DR591- | 991 | CAGGTCCAGCTCCAGGAATCTGGTGGCGGAAGCGTGCAGGCCGGTGGGTCCCTG |
| hIL27 | | AGACTGAGCTGTACCGCGAGCGGAGCTATCGCTTCTGGGTACATTGACTCCCGG |
| Ra_VH | | TGGTGCATGGCGTGGTTCCGTCAGGCTCCAGGTAAGGAGCGGGAGGGCGTTGCT |
| H19 | | GCGATCTGGCCAGGAGGGGGATTGACTGTGTACGCTGACTCTGTCAAGGGTCGC |
| | | TTTACTATCTCCCGCGACCACGCAAAGAATACCTTGTACTTGCAAATGAACAAT |
| | | CTGAAGCCTGAGGACACCGCCATGTATTACTGTGCCGCTGGTAGCCCACGCATG |
| | | TGCCCCAGTCTGGAGTTCGGGTTCGATTATTGGGGGCAGGGCACGCAGGTGACT |
| | | GTGTCCTCTGGTGGAGGCAGCCAAGTGCAGTTGCAGGAGTCAGGGGGAGGGAGC |
| | | GTGCAGGCGGGAGGTTCCCTGCGGCTGTCCTGTCGCGCGTCCGGTTCCACCTAC |
| | | TCTAACTATTGCCTGGGTTGGTTCCGCCAGATCACCGGCAAGGAGCGCGAGGGA |
| | | GTGGCGGTTATCAACTGGGTCGGCGGTATGCTGTACTTTGCAGACTCTGTTAAA |
| | | GGACGCTTCACTGTGAGCCAGGACCAGGCAAAGAATACTGTGTACTTGCAGATG |
| | | AACTCCCTGAAGCCAGAAGACACCGCCATGTATTACTGTGCTGCGGAATCTGTG |
| | | AGTTCCTTCAGTTGCGGTGGGTGGCTCACTAGACCCGATAGAGTCCCATATTGG |
| | | GGACAGGGTACTCAGGTGACTGTTAGCTCC |
| DR591- | 992 | CAGGTGCAGTTGCAGGAGAGTGGCGGTGGCAGCGTGCAGGCGGGGGGGAGCCTG |
| hIL27 | | AGGCTGTCCTGTACTGCCTCTGGTGCAATCGCTTCAGGTTACATTGACTCACGC |
| Ra_VH | | TGGTGCATGGCTTGGTTTCGCCAGGCTCCAGGCAAGGAGCGCGAGGGTGTGGCC |
| H19 | | GCTATCTGGCCGGGTGGAGGCCTCACCGTGTACGCCGATAGTGTCAAGGGACGC |
| | | TTTACAATCTCCCGTGACCATGCGAAGAACACATTGTATCTTCAGATGAATAAC |
| | | CTCAAACCCGAGGACACCGCTATGTATTACTGTGCAGCCGGAAGCCCTCGTATG |
| | | TGTCCAAGCCTGGAGTTCGGCTTTGATTACTGGGGACAAGGCACCCAGGTGACA |
| | | GTTTCTAGTGGAGGCTCTGGCGGTTCAGGAGGCTCCGGGCAAGTGCAGTTGCAG |
| | | GAATCCGGGGGTGGCTCCGTGCAGGCTGGGGGCTCTCTGCGGCTGTCTTGTCGT |
| | | GCATCTGGTTCCACATATAGCAACTACTGCCTGGGCTGGTTCCGCCAGATTACG |
| | | GGCAAGGAAAGAGAAGGTGTTGCAGTGATAAATTGGGTAGGTGGAATGCTGTAC |
| | | TTTGCCGACTCAGTGAAAGGGCGCTTTACAGTGAGCCAGGACCAGGCCAAGAAC |
| | | ACAGTCTACTTGCAGATGAACAGCCTCAAGCCTGAAGACACCGCAATGTACTAT |
| | | TGTGGTGCCGAGAGCGTCTCATCCTTTTCCCGCGGAGGGTGGCTGACCCGTCCA |
| | | GACCGCGTCCCCTACTGGGGCCAGGGCACCCAGGTGACTGTGTCCTCT |
| DR591- | 993 | CAAGTGCAGCTGCAAGAGTCCGGTGGAGGCAGCGTACAAGCGGGTGGCTCACTG |
| hIL27 | | CGCCTGAGTTGTACCGCTTCTGGTGCAATCGCAAGCGGCTACATTGATTCCAGA |
| Ra_VH | | TGGTGCATGGCATGGTTTCGCCAAGCCCCAGGCAAGGAAAGGGAGGGCGTGGCC |
| H20 | | GCAATCTGGCCTGGGGGTGGACTGACCGTGTATGCCGACAGCGTGAAGGGGCGC |
| | | TTTACCATCTCCCGTGACCACGCTAAGAACACACTGTACTTGCAGATGAATAAC |
| | | CTCAAGCCTGAAGACACTGCTATGTACTATTGCGCTGCCGGAAGTCCCCGCATG |
| | | TGTCCTAGTCTGGAGTTTGGTTTTGATTACTGGGGCCAGGGCACCCAGGTGACC |
| | | GTTTCAAGCGGAGGGGGCAGTCAGGTTCAGCTCCAGGAATCTGGAGGTGGGCTT |
| | | GTCCAGCCAGGGGGCAGCCTCCGCCTGTCCTGTGCAGCGTCTGGGTTTACTTTC |
| | | AGCTCCTATCCCATGTCTTGGGTTAGACAGGCACCAGGCAAGGGCCTGGAGTGG |
| | | GTGTCCACAATCTCAAGCGGGGGCGATACAACCCTCTATGCCGATTCCGTGAAG |
| | | GGCAGATTCACAAGCTCTAGGGACAACGCCAAGAATACCTTGTACCTCCAGCTG |
| | | AACTCCCTGAAGACCGAGGACACTGCGATGTATTACTGCGCTAAGCGCATTGAT |
| | | TGTAACTCTGGGTATTGTTACAAGCGCTCTTATTGGGGACAGGGCACGCAGGTG |
| | | ACTGTCAGTTCC |
| DR591- | 994 | CAGGTGCAGTTGCAAGAGAGCGGCGGAGGCTCCGTTCAGGCAGGGGGCTCCTTG |
| hIL27 | | CGCTTGTCTTGCACCGCATCCGGGGCCATTGCCTCAGGCTATATCGACACCCGC |
| Ra_VH | | TGGTGTATGGCTTGGTTCCGGCCAAGCGCCGGGTAAGGAGCGTGAGGGGGGGCA |
| H20 | | GCCATCTGGCCCGGAGGCGGTTTGACCGTGTATGCCGACTCTGTGAAGGGCCGG |
| | | TTCACCATCTCTAGGGACCATGCGAAGAACACACTGTATCTCCAGATGAATAAC |
| | | CTGAAGCCCGAAGACACCGCGATGTATTACTGTGCCGCTGGCTCCCCCAGGATG |
| | | TGCCCCTCATTGGAGTTCGGTTTCGACTATTGGGGGCAGGGAACTCAAGTGACC |
| | | GTAAGCTCCGGGGGGTCCGGCGGTAGTGGAGGCTCTGGGCAGGTGCAGTTGCAG |
| | | GAGTCAGGCGGGGGCTTGGTGCAGCCCGGCGGTAGCCTGAGGCTGAGTTGCGCT |
| | | GCCTCAGGCTTTACCTTTTCCAGCTACCCTATGTCTTGGGTGCGCCAGGGTCCG |
| | | GGCAAGGGCTTGGAGTGGGTGTCCACCATCTCCAGCGGAGGCGATACGACCCTC |
| | | TATGCGGATTCCGTCAAAGGGGGCTTTACCTCCAGCCGCGATAACGCCAAGAAC |
| | | ACCTTGTACCTCCAGGTGAACTCCCTGAAGACCGAGGATACCGCAATGTACTAT |
| | | TGTGCAAAGCGGATTGATTGTAACAGCGGCTATTGCTACAAGAGGTCTTACTGG |
| | | GGCCAGGGCACCCAGGTGACAGTCTCCAGT |
| DR591- | 995 | CAGGTCCAGCTCCAGGAGAGCGGTGGCGGTTCCGTGCAGGCTGGTGGCAGTCTT |
| hIL27 | | CGTCTCTCCTGTACCGCGTCCGGTGCCATCGCCAGTGGCTACATTGATTCTCGC |
| Ra_VH | | TGGTGCATGGCATGGTTCCGCCAAGCTCCGGGCAAGGAGCGCGAGGGGGTCGCT |
| H21 | | GCCATTTGGCCAGGGGGGGGCTGACCGTGTACGCCGGACTCTGTGAAGGGTGGG |
| | | TTTAGGATCAGCCGGGACCATGCTAAGAATACGCTCTACCTGCAAATGAACAAT |
| | | CTGAAACCCGAGGACACAGCAATGTACTATTGCGCAGCCGGAAGTCCTCGCATG |
| | | TGCCCATCTTTGGAGTTTGGCTTCGATTATTGGGGCCAGGGCACCCAGGTCACC |
| | | GTGTCCAGTGGGGGGGGTTCTCAGGTGCAGTTGCAGGAAAGTGGAGGTGGCCTT |
| | | GTCCAACCTGGAGGGAGTCTGCGTCTCTCCTGCGCTGCCTCCGGGTTCACCTTT |
| | | AGTCTGTCCAGTATGTCTTGGGTCCGCCAAGCGCCCGGAAAGGGTCTGGAGTGG |
| | | GTCAGCGCGATTAGCTCCGGTGGAGCCAGTACTTATTACACCGACAGCGTGAAA |
| | | GGACGGTTTACCATCTCCCGCGATAATGCTAAGAATATGCTCTACTTGCAGCTG |
| | | AACAGCCTGAAGACCGAGGACACAGCGATGTATTACTGTGCCAAGGGAGGTTCT |
| | | GGCTATGGTGATGCCAGCCGCATGACCTCCCCTGGCTCCCAGGGAACTCAGGTT |
| | | ACTGTGTCCTCC |
| DR591- | 996 | CAAGTGCAGTTGCAGGAGAGCGGTGGCGGTAGCGTCCAAGCTGGGGGCAGTTTG |
| hIL27 | | CGCCTGAGCTGTACTGCGTCTGGCGCTATTGCGAGCGGTTACATTGATTCCAGG |
| Ra_VH | | TGGTGTATGGCCTGGTTCAGGCAGGCACCGGGTAAAGAGCGTGAGGGGGTGGCA |
| H21 | | GCCATTTGGCCTGGCGGTGGACTGACCGTCTACGCGGACTCTGTCAAGGGACGT |
| | | TTCACGATCAGCCGCGATCACGCTAAAAACACCCTCTACCTCCAGATGAATAAC |
| | | CTGAAGCCTGAGGACACAGCTATGTATTACTGTGCTGCGGGCAGTCCCAGAATG |
| | | TGTCCCAGCCTGGAGTTCGGCTTCGACTACTGGGGGCAAGGCACCCAGGTGACA |
| | | GTGTCCAGCGGGGGCTCCGGCGGGTCTGGGGGCAGCGGCCAGGTGCAACTGCAA |
| | | GAGTGTGGCGGTGGACTTGTTCAGCCAGGGGGATCTCTGAGGCTCTCCTGCGCC |
| | | GCTTCCGGTTTTACCTTCTCCCTCAGCAGTATGTCCTGGGTCCGTCAGGCTCCA |
| | | GGCAAAGGCCTGGAATGGGTTTCCGCTATTAGCTCTGGGGGTGCCTCCACCTAC |
| | | TATACGGACTCAGTCAAAGGCCGTTTCACGATGTCCAGGGACAATGCTAAGAAT |
| | | ATGCTCTACCTCCAGTTGAACTCTCCGAAAACGGAGGACACCGCTATGTACTAT |
| | | TGCGCCAAAGGGGGTTCCGGCTACGGCGACGCCTCCGGGATGACATCCCGTGGG |
| | | TCCCAGGGAACCCAGGTGACCGTGTCCAGC |
| DR591- | 997 | CAGGTGCAGTTGCAGGAGTCCGGGGGAGGCTCCGTGCAAGCTGGCGGTAGCTTG |
| hIL27 | | CGGCTGAGTTGCACTGCCAGCGGTGCCATCGCGAGCGGGTACATCGACAGCAGG |
| Ra_VH | | TGGTGCATGGCCTGGTTCCGTCAAGCGCCAGGAAAGGAGAGGGAGGGCGTCGCA |
| H32 | | GCCATCTGGCCGGGCGGAGGCGTGACCGTGTACGCAGATTCCGTCAAAGGCCGT |
| | | TTCACCATCTCCCGCGACCACGCCAAGAACACCCTGTATCTTCAGATGAATAAC |
| | | CTGAAACCCGAGGACACCGCCATGTACTATTGTGCTGGGGGCTCACCCCGGATG |
| | | TGTCCCTCTCTGGAGTTCGGCTTCGATTATTGGGGCCAGGGCACCCAGGTCACC |
| | | GTGTCAAGTGGTGGCGGTTCCCAGGTGCAGCTGCAAGAGAGCGGCGGTGGCAGG |
| | | GTGCAGGGGGGCGGTAGCTTGCGCCTGAGCTGTCGCGCCTCCGGCAGCACTTAC |
| | | AGCAACTACTGCCTGGGCTGGTTCCGTCAGACTACAGGTAAGGAGCGTGAGGGC |
| | | GTAGCGGTAATCAACTGGGTCGGGGGAATGCTTTACTTTGCGGACAGTGTGAAA |
| | | GGCCGTTTCACCGTGTCTCAGGACCAGGCTAAGAACACTGTGTACCTCCAGATG |
| | | AACTCTCTGAAGCCTGAGGACACGGCCATGTATTACTGTGCAGCGGAGTCCGTT |
| | | AGCTCCTTCTCTTGTGGCGGATGGCTGACCCGGCCAGACCGCGTGCCATATTGG |
| | | GGCCAAGGGACCCAAGTGACCGTGTCATCC |
| DR591- | 998 | CAGGTGCAGCTCCAGGAGTCCGGCGGTGGGTCCGTGCAGGCTGGTGGATCTCTT |
| hIL27 | | AGACTGTCCTGTACCGCTTCTGGAGCGATTGCGTCAGGTTACATTGACAGTAGG |
| Ra_VH | | TGGTGTATGGCTTGGTTCCGTCAGGCTCCAGGCAAGGAACGCGAGGGGGTTGCT |
| H22 | | GCCATTTGGCCAGGAGGCGGTCTGACTGTCTACGCTGACAGCGTAAAGGGCCGC |
| | | TTTACCATCTCTCGGGATCACGCGAAAAATACACTGTACCTCCAGATGAATAAC |
| | | CTGAAGCCGGAGGACACCGCCATGTATTACTGCGCTGCCGGGAGCCCCCGTATG |
| | | TGCCCTTCCCTGGAGTTCGGATTTGACTATTGGGGCCAGGGCACCCAAGTGACC |
| | | GTCAGCTCCGGTGGCTCTGGCGGTTCCGGGGGCTCTGGCCAAGTGCAGCTTCAG |
| | | GAGTCAGGGGGTGGCTCTGTGCAGGCCGGTGGCAGCCTGCGTCTGAGCTGCCGT |
| | | GCCAGCGGCTCCTACCTATTCCAACTACTGCCTGGGGGGTTTCGGCAGACCACA |
| | | GGAAAGGAAAGAGAGGGTGTGGCAGTTATCAACTGGGTGGGATGGGATGTGTAC |
| | | TTCGCCGACTCCGTCAAGGGGCGCTTCACCGTCAGTCAGGACCAGGCAAAGAAC |
| | | ACCGTGTACCTCCAGATGAACTCCCTGAAGCCTGAGGATACCGCCATGTATTAC |
| | | TGCGCCGCTGAGTCTGTGTCATCTTTCTCATGCGGTGGCTGGCTCACCCGCCCA |
| | | GACCGCGTGCCTTATTGGGGCCAGGGCACACAGGTCACCGTCAGGTCA |
| DR591- | 999 | CAGGTTCAACTCCAGGAAAGCGGCGGGGGCTCTGTGCAAGCTGGCGGAAGTTTG |
| hIL27 | | CGTCTGTCCTGCACCGCCTCTGGAGCCATCGCCAGCGGCTACATCGACTCAAGA |
| Ra_VH | | TGGTGTATGGCCTGGTTCAGACAGGCTCCAGGAAAAGAACGCGAAGGCGTGGCC |
| H23 | | GCTATCTGGCCCGGAGGTGGCCTGACCGTGTACGCTGACTCCGTCAAGGGCCGC |
| | | TTCACCATCTCCCGTGACCACGCAAAGAACACACTGTACCTCCAGATGAATAAC |
| | | CTGAAGCCCGAGGATACCGCCATGTACTATTGCGCTGCGGGCTCCCCTCGGATG |
| | | TGTCCCAGCCTGGAGTTCGGCTTCGACTATTGGGGACAGGGAACTCAGGTCACA |
| | | GTCAGCTCCGGCGGAGGCTCTCAGGTCCAGCTCCAGGAATCAGGTGGAGGCTCT |
| | | GTTCAGGCGGGCGGATCTCTTCGCCTGTCATGCCGTGCATCCAGATCCCCGTAT |
| | | GGCAACTATTGCCTGGGGGGTTCCGCCAGAGTACCAGGTAAGGAGCGCGAGGGC |
| | | GTGGGTGTTATCAACTGGGTCGGCGGAATGCTGTATTTTGCCGATTCCGTGAAG |
| | | GGTCGTTTCACAGTGAGCCAGGATCACGCCAAGAACACCGTGACCTTGCAGATG |
| | | AACTCCCTGAAGCCTGAAGATACCGCCATGTATTACTGTGCTGCGGAGAGCGTG |
| | | TCCTCTTTCTCTTGCGGCGGTTGGCTGACCAGGCCTGATCGTGTCCCCTACTGG |
| | | GGGCAGGGCACCCAGGTGACCGTCTCTAGC |
| DR591- | 1000 | CAAGTGCAGTTGCAGGAGTCAGGCGGAGGCTCTGTCCAGGCTGGAGGCTCTCTG |
| hIL27 | | AGACTCTCCTGCACCGCATCCGGGGCCATCGCCAGGGGATATATCGACTCCAGA |
| Ra_VH | | TGGTGTATGGCGTGGTTTCGCCAAGCTGCGGGGAAGGAGCGTGAGGGTGTCGCT |
| H23 | | GCGATCTGGCCTGGCGGGGGACTGACCGTGTATGCTGATTCCGTAAAGGGCCGT |
| | | TTCACCATCAGCCGCGACCATGCCAAGAACACACTCTACCTCCAGATGAACAAT |
| | | CTTAAACCCGAGGACACTGCAATGTACTATTGTGCGGCTGGTTCTCCCCGCATG |
| | | TGTCCTTCCCTGGAGTTCGGCTTCGACTACTGGGGGCAGGGCACCCAGGTCACC |
| | | GTGAGTAGCGGCGGGAGCGGCGGGAGCGGTGGCTCCGGCCAGGTGCAGCTTCAA |
| | | GAGTCCGGGGGTGGCTCCGTGCAAGCTGGTGGAAGTCTTCGCCTGAGTTGCCGC |
| | | GCGTCCCGCTCCCCTTACGGCAATTATTGCCTGGGATGGTTTCGCCAATCCACC |
| | | GGGAAGGAGCGCGAGGGCGTGGCAGTAATCAACTGGGTGGGGGGTATGCTGTAC |
| | | TTTGCCGATTCTGTGAAGGGCAGATTTACCGTGTCTCAAGACCACGCCAAGAAC |
| | | ACTGTCACCTTGCAGATGAACTCACTGAAGCCAGAGGATACCGCTATGTATTAC |
| | | TGCGCTGCCGAATCAGTCAGCTCCTTCTCTTGCGGTGGCTGGCTGACCCGCCCT |
| | | GATAGGGTCCCCTATTGGGGCCAGGGCACCCAGGTCACCGTGTCCAGT |
| DR591- | 1001 | CAGGTCCAGCTCCAGGAGTCCGGCGGAGGTTCTGTCCAGGCCGGTGGATCACTG |
| hIL27 | | CGTCTGTCTTGCACCGCCAGGGGCGCAATCGCGTCTGGCTATATCGACAGCCGT |
| Ra_VH | | TGGTGCATGGCTTGGTTTCGCCAGGCTCCCGGCAAGGAAAGGGAAGGGGTGGCC |
| H24 | | GCGATTTGGCCTGGGGGGGGTGTGACCGTCTACGCCGACAGCGTGAAGGGCCGC |
| | | TTTACCATCTCCCGCGACCACGCCAAGAACACATTGTACCTGCAAATGAATAAC |
| | | CTCAAGCCTGAGGACACAGCGATGTATTACTGTGCCGCTGGCTCACCCCGCATG |
| | | TGTCCGTCCCTGGAGTTTGGGTTTGATTATTGGGGCCAGGGCACACAGGTGACC |
| | | GTGTCATCTGGAGGCGGTTCCCAGGTCCAGTTGCAGGAGTCTGGCGGAGGTCTG |
| | | GTGCAGCCAGGAGGCAGCCTGCGGCTGTCTTGTGCTGCGTCTGGTTTCACCTTC |
| | | TCTCACAGCGGCATGTCTTGGGTGCGCCAAGCACCGGGCAAGGGGCTGGAGTGG |
| | | GTCTCCACAATTAACTCCGGGGGGCCTCCACCTATTACACCGACCTCTGTAAAG |
| | | GGTCGGTTTACCATTAGCCGCGATAACGCCAAAAATATGTTGTATTTGCAGCTT |
| | | AACTCCCTGAAGACCGAAGATACCGCTATGTATTACTGGGCAAAGGGTGGGAGC |
| | | GGTTACGGCGACGCCTCCCGCATGACCTCTCCGGGATCTCAGGGAACCCAAGTT |
| | | ACCGTGAGTAGT |
| DR591- | 1002 | CAGGTGCAGCTCCAGGAATCTGGAGGGGGTTCCGTGCAGGCAGGCGGTTCCCTG |
| hIL27 | | AGGCTTAGTTGTACCGCCTCTGGGGCTATCGCCTCCGGCTACATTGACTCTCGC |
| Ra_VH | | TGGTGTATGGCCTGGTTCAGGCAGGCTCCAGGTAAGGAGCGCGAGGGTGTCGCG |
| H24 | | GCTATTTGGGCTGGTGGCGGACTGACTGTGTACGCCGATTCTGTGAAGGGCCGG |
| | | TTTACTATTAGCAGGGATCATGCGAAAAATACCCTTTACTTGCAGATGAACAAT |
| | | CTGAAGCCGGAGGACACGGCCATGTACTATTGCGCCGCTGGCTCACCTAGAATG |
| | | TGTCCTAGCTTGGAGTTCGGGTTTGATTACTGGGGCCAGGGAACCCAGGTGACC |
| | | GTCTCTAGCGGTGGGAGCGGGGGGAGCGGGGGAAGCGGGCAGGTTCAGTTGCAG |
| | | GAATCAGGAGGCGGACTGGTGCAACCGGGAGGGTCCCTGCGCCTGTCTTGTGCT |
| | | GCCTCTGGTTTTACTTTTAGCCATAGCGGGATGTCCTGGGTTCGCCAGGCTCCA |
| | | GGCAAGGGCCTGGAGTGGGTATCTACCATCAACTCTGGAGGTGCCAGCACCTAT |
| | | TACACCGACAGCGTGAAGGGGAGGTTCACCATCTCCCGCGACAACGCTAAGAAC |
| | | ATGCTGTACCTCCAGGTCAACTCTCTGAAGACCGAGGACACCGCTATGTATTAC |
| | | TGCGCGAAGGGCGGTTCCGGTTACGGAGACGCCTCCCGCATGACCAGTCCGGGT |
| | | TCCCAAGGTACACAGGTGACCGTGTCTAGC |
| DR592- | 1003 | CAGGTTCAACTCCAGGAGTCCGGGGGGGGTTCCGTGCAAGCTGGAGGTTCACTG |
| hIL27 | | CGCCTGAGCTGCACTGCGCCAGGCTTCACATCAAATTCTTGTGGTATGGATTGG |
| Ra_VH | | TATCGGCAAGCCCCCGGAAAAGAGCGGGAGTTCGTGTCCAGTATTAGCACAGAC |
| H1 | | GGTACGACCGGATATGCGGACTCTGTGAAAGGACGCTTCACGATTTCCAAAGAC |
| | | AAGGCCAAGGACACCGTGTACCTTCAGATGAACTCACTGAAACCAGAGGACACG |
| | | GGCATGTACTCCTGTAAGACGAAGGACGGAACAATCGCCACTATGGAATTGTGC |
| | | GATTTCGGCTATTGGGGCCAGGGGACGCAGGTTACCGTTTCTTCAGGGGGAGGG |
| | | AGTCAGGTTCAGTTGCAGGAGTCCGGCGGTGGGTTGGTGCAACCTGGGGGAAGC |
| | | CTGCGTCTTTCTTGTGCAGCCAGCGGCTTCACCTTCAGTTCATACCCCATGAGC |
| | | TGGGTCCGTCAGGCCCCCGGTAAAGGGGTGGAGTGGATGTCAACCATCTCTGCC |
| | | GGTGGCGACACCACGCTTTACGCTGACTCCGTCAAAGGCAGATTCACCAGTAGC |
| | | AGAGACAACGCGAAGAACACCCTCTATTTGCAGCTTAACTCCCTCAAGACCGAG |
| | | GACGCAGCTATCTACTATTGTGCTAAGAGGATTGACTGTAACAGGGGCTACTGT |
| | | TATCGTCGCAACTATTGGGGCCAGGGCACTCAGGTCACAGTGTCTTCC |
| DR592- | 1004 | CAGGTCCAGGTCCAGGAGAGGGGCGGAGGGTCTGTGCAGGGGGGGGGTAGTTTG |
| hIL27 | | CGCTTGAGCTGTACCGCACCCGGATTCACCTCCAACAGCTGCGGTATGGACTGG |
| Ra_VH | | TATCGCCAGGCTCCTGGTAAAGAGCGTGAGTTCGTGTCTTCAATCAGCACCGAC |
| H1 | | GGTACAACTGGGTATGCCGATTCCGTGAAAGGAAGATTCACCATCTCCAAGGAC |
| | | AAGGCCAAAGACACAGTTTATCTTCAGATGAACAGTCTGAAGCCCGAGGACACC |
| | | GGCATGTATTCTTGTAAGACTAAGGACGGGACGATTGCGACAATGGAACTGTGC |
| | | GACTTCGGTTACTGGGGCCAGGGGACTCAGGTAACTGTGTCAAGGGGAGGCTCT |
| | | GGCGGAAGTGGAGGCTCAGGACAGGTGCAGCTCCAGGAGTCAGGAGGGGGCCTG |
| | | GTCCAGCCGGGCGGGTCTCTGCGCCTCTCTTGCGCTGCCTCTGGGTTCACATTC |
| | | AGCAGTTACCCTATGTCCTGGGTACGCCAGGCTCCAGGCAAAGGTCTGGAGTGG |
| | | ATCTCCACCATCAGTGCCGGAGGCGATACCACTCTGTATGCCGACAGCGTGAAG |
| | | GGACGCTTTACCTCCTCACGCGATAATGCCAAGAACACTCTGTACCTGCAACTC |
| | | AATAGTCTGAAGACTGAGGACGCGGCCATCTATTACTGCGCCAAGCGCATTGAT |
| | | TGCAATAGTGGCTACTGTTACCGGCGCAACTATTGGGGACAGGGTACTCAGGTA |
| | | ACAGTCAGCTCC |
| DR592- | 1005 | CAGGTCCAACTGCAAGAGTCTGGAGGGGGCTCTGTGCAGGCCGGAGGCTCCCTT |
| hIL27 | | CGCCTGTCTTGTACCGCCCCAGGTTTTACCTCAAACTCGTGGGGCATGGATTGG |
| Ra_VH | | TATAGACAGGCTCCTGGGAAAGAGAGGGAGTTCGTGTCTTCCATCAGCACCGAC |
| H2 | | GGTACAACCGGCTACGCGGATTCCGTGAAAGGTCGCTTTACCATTTCAAAGGAC |
| | | AAAGCTAAAGATACCGTGTACCTCCAGATGAACTCACTGAAGCCCGAGGACACT |
| | | GGCATGTACTCTTGTAAGACCAAGGATGGCACCATCGCCACTATGGAGTTGTGT |
| | | GATTTCGGGTACTGGGGCCAGGGCACACAAGTGACCGTCTCCAGCGGTGGAGGC |
| | | AGCCAGGTGCAGCTTCCAAGAGTCCGGGGGGGGCTGGTCCAGCCAGGAGGCTCA |
| | | CTCCGCCTGTCCTGTGCGGCTTCCGGGTTTACCTTCTCACTGTCCGGCATGTCC |
| | | TGGGTCCGCCAGGCCCCCGGCAAAGGCCTTGAGTGGGTGTCCGCTATTAGCTCC |
| | | GGTGGAGCCAGCACTTACTATACTGACTCTGTTAAAGGCCGGTTTACAATCTCC |
| | | CGTGACAATGCCAAGAACATCCTGTATCTTCAGCTGAACAGTCTCAAGACAGAG |
| | | GATACTGCCATGTACTATTGTGCTAAAGGTGGCTCCGGCTACGGCGACGCGAGC |
| | | CGTATGACTTCTCCTGGCTCCCAGGGCACTCAGGTGACTGTTTCCAGC |
| DR592- | 1006 | CAGGTCCAGGTTCAGGAGAGCCGGGGGGGGAGCGTGCAGGCCGGGGGCTCTCTG |
| hIL27 | | CGCCTGTCCTGCACAGCGCCCGGTTTTTACGTCCAACTCTTGCGGCATGGATGG |
| Ra_VH | | TATCGCCAAGCGCCTGGCAAGGAAAGAGAGTTTGTCAGCTCCATCAGCACCGAT |
| H2 | | GGCACTACCGGCTACGCGGATTCTGTAAAGGGTCGTTTCACCATCAGTAAGGAT |
| | | AAGGCTAAGGACACGGTGTACCTCCAGATGAACTCCCTCAAGCCCGAGGATACC |
| | | GGGATGTACTCTTGCAAGACCAAGGACGGCACCATCGCCACTATGGAGCTTTGC |
| | | GACTTCGGCTATTGGGGCCAGGGAACTCAGGTGACCTGTGTCAAGTGGGGGTCA |
| | | GGAGGTAGCGGAGGTTCTGGCCAGGTGCAGCTTCAGGAATCTGGGGGTGGGCTC |
| | | GTCCAGCCGGGAGGCTCTCTGCGCCTGAGCTGCGCAGCCTCTGGCTTTACCTTT |
| | | TCTCTGTCTGGCATGTCTTGGGTCCGCCAGGCACCTGGAAAGGGGCTGGAGTGG |
| | | GTTTCCGCCATCTCCTCTGGTGGCGCATCCACATATTACACCGACTCTGTCAAG |
| | | GGCCGCTTCACCATTTCACGCGATAATGCCAAGAACATCCTGTACCTTCAGCTG |
| | | AACTCCCTGAAGACCGAGGACACAGCTATGTATTACTGCGCTAAGGGAGGGTCC |
| | | GGCTACGGGGATGCGAGCAGAATGACCAGCCCTGGTTCACAAGGCACCCAGGTA |
| | | ACTGTTAGCAGT |
| DR592- | 1007 | CAAGTCCAACTTCAGGAGTCCGGGGGGGGCTCTGTGCAGGCTGGAGGGTCTCTG |
| hIL27 | | CGCCTGTCCTGCACTGCACCTGGGTTCACTTCAAACTCTTGGGGCATGGACTGG |
| Ra_VH | | TATCGCCAGGCCCCAGGCAAGGAACGTGAGTTTGTGTCCAGCATTTCAACTGAC |
| H3 | | GGCACAACCGGGTATGCTGATAGCGTGAAGGGTAGGTTTACCATCTCCAAGGAT |
| | | AAGGCCAAGGATACCGTGTACTTGCAGATGAACTCCCTCAAACCTGAGGATACC |
| | | GGAATGTATAGCTGCAAGACCAAAGATGGAACCATCGCTACTATGGAGCTGTGT |
| | | GACTTTGGCTACTGGGGGCAGGGCACCCAGGTGACCGTGTCCAGCGGCGGTGGA |
| | | TCTCAGGTCCAGCTCCAGGAGAGCGGGGGGGGGTCTGTGCAGGCAGGGGGGTCC |
| | | CTGCGTCTGTCATGCGTCGCGTCAGGCTACGTCTCCTGCGATTACTTCCTGCCC |
| | | TCCTGGTATCGCCAGGCCCCCGGCAAGGAAAGGGAGTTCGTCAGTATCATTGAC |
| | | GGAACCGGCTCCACCAGCTACGCTGCCTCCGTCAAGGGCCGGTTCACCGCCTCT |
| | | GAGGACAAAGGGAAGAACATCGCCTACCTCCAGATGAACAGTCTGAAACCAGAG |
| | | GATACTGCAATGTACTATTGCAAGGCCTCCTGTGTCCGTGGCCGTGCCGTTTCT |
| | | GAGTACTGGGGCCAAGGGACTCAGGTGACTGTCTCCTCG |
| DR592- | 1008 | CAGGTTCAGTTGCAGGAGAGTGGAGGGGGCTCCGTGCAGGCCGGTGGCTCCCTG |
| hIL27 | | AGGCTGTCCTGCACAGCTCCCGGCTTCACCAGTAATTCGTGTGGCATGGACTGG |
| Ra_VH | | TATCGCCAGGCCCCCGGTAAGGAAAGAGAGTTTGTGTCCAGTATCTCAACCGAC |
| H3 | | GGCACCACTGGCTATGCGGACTCCGTCAAGGGCAGGTTCACTATGTCCAAGGAT |
| | | AAGGCCAAAGACACAGTGTACCTCCAAATGAACTCTCTCAAGCCCGAGGACACG |
| | | GGCATGTATTCCTGTAAAACCAAGGACGGCACCATCGCTACTATGGAGCTGTGT |
| | | GATTTCGGATACTGGGGCCAGGGAACCCAGGTGACCGTGTCAAGCGGAGGTAGT |
| | | GGTGGCTCAGGCGGGTCCGGGCAGGTCCAACTCCAGGAGTCTGGAGGCGGGAGT |
| | | GTGCAGGCTGGTGGCTCCCTGCGGCTGAGCTGCGTGGCTTCCGGGTATGTTTCT |
| | | TGCGATTACTTCCTGCCGAGCTGGTACAGGCAGGCTCCCGGTAAGGAGCGTGAG |
| | | TTCGTTTCCATCATTGACGGCACCGGGAGCACAAGCTACGCCGCTAGTGTGAAG |
| | | GGACGCTTCACGGCAAGCGAAGACAAGGGCAAAAACATCGCGTACTTGCAGATG |
| | | AACTCCTTGAAGCCCGAGGACACCGCTATGTATTACTGTAAAGCCTCATGCGTC |
| | | AGAGGCAGGGCTGTCTCAGAGTACTGGGGTCAGGGCACGCAAGTGACAGTCTCC |
| | | TCC |
| DR592- | 1009 | CAAGTGCAACTCCAGGAGTCCGGCGGTGGGTCTGTGCAGGCTGGGGGCTCACTG |
| hIL27 | | CGTCTGAGCTGCACCGCTCCCGGTTTTACCAGTAACTCTTGTGGTATGGACTGG |
| Ra_VH | | TATCGCCAGGCCCCCGGCAAGGAGAGGGAATTTGTCAGTTCCATGTCTACAGAC |
| H4 | | GGCACTACAGGATACGCCGACTCCGTCAAGGGGCGGTTCACCATCTCAAAAGAT |
| | | AAAGCAAAGGACACAGTCTACCTTCAGATGAACAGCCTGAAACCTGAGGACACC |
| | | GGAATGTATAGCTGCAAGACAAAGGATGGAACAATCGCCACGATGGAGCTGTGT |
| | | GACTTCGGATACTGGGGCCAGGGGACACAGGTCACTGTGTCCTCTGGAGGGGGG |
| | | TCCCAGGTGCAGCTCCAGGAGAGCGGCGGAGGCCTGGTGCAGCCCGGCGAGTCC |
| | | CTTAGGCTGAGCTGTACGGCGAGCGGCTTTACTTTCAGCAACTACGCCATGAGC |
| | | TGGGTCCGTCAAGCGCCGGGGAAGGGGCTGGAGTGGGTTTCTGGCATCAACGTG |
| | | GCATACGGCATCACCTCCTATGCTGATAGCGTCAAGGGCCGCTTCACCATTTCC |
| | | AGAGATAATACCAAGAATACCCTCTACCTCCAGCTCAATTCCCTGAAGACCGAG |
| | | GACACTGCTATTTATTACTGTGTGAAGCACCCTGGCACCACAATTCCTCGCGGC |
| | | TTTATCAGCTATACAAAGAGAGGCCAGGGAACTCAGGTCACCGTCAGCTCT |
| DR592- | 1010 | CAGGTCCAGTTGCAGGAATCTGGGGGGGGATCAGTGCAGGCCGGGGGCTCCCTG |
| hIL27 | | CGTCTTTCTTGCACAGCCCCCGGCTTTACCTCCAATTCCTGTGGCATGGATTGG |
| Ra_VH | | TATCGGCAGGCCCCCGGCAAGGAGCGCGAGTTTGTCTCTAGCATTAGCACCGAC |
| H4 | | GGCACCACTGGTTACGCTGACTCAGTGAAGGGACGGTTCACCATTTCCAAGGAC |
| | | AAGGCTAAAGACACAGTCTACCTTCAGATGAACAGTCTGAAGCCTGAGGATACA |
| | | GGGATGTACTCCTGTAAGACCAAGGACGGCACCATCGCAACGATGGAGCTGTGC |
| | | GACTTCGGCTATTGGGGGCAAGGGACCCAGGTGACTGTGTCCTCTGGGGGCTCC |
| | | GGGGGCAGCGGAGGCTCCGGCCAAGTGCAACTCCAGGAGAGCGGCGGTGGACTG |
| | | GTGCAGCCAGGCGAGAGCCTCCGGCTTTCCTGCACCGCAAGCGGGTTCACTTTT |
| | | AGCAACTACGCTATGTCCTGGGTAAGGCAGGCTCCAGGTAAGGGCCTGGAGTGG |
| | | GTGAGCGGTATTAACGTGGCATACGGCATCACTTCCTACGCGGACAGCGTCAAG |
| | | GGCCGTTTTACTATCAGCAGGGATAATACTAAGAATACTCTGTACTTGCAGCTG |
| | | AACAGCCTGAAGACTGAAGATACTGCCATCTATTACTGCGTGAAGCACTCTGGA |
| | | ACAACCATTCCACGGGGCTTCATCAGTTACACCAAGCGGGGCCAGGGCACTCAG |
| | | GTCACCGTGTCTTCC |
| DR592- | 1011 | CAAGTGCAGGTCCAGGAGTCCGGCGGTGGAAGCGTGCAGGCCGGAGGCTCTCTG |
| hIL27 | | CGCTTGAGTTGCACCGCCCCTGGATTCACATCTAACTCCTGTGGTATGGATTGG |
| Ra_VH | | TACAGGCAGGCACCAGGCAAGGAACGTGAGTTCGTGTCTTCCATCAGTACCGAC |
| H5 | | GGTACTACCGGCTACGCGGACTCCGTCAAAGGTAGATTTACGATCAGCAAAGAT |
| | | AAGGCTAAGGATACCGTGTATCTCCAGATGAACTCCCTGAAACCTGAGGATACA |
| | | GGTATGTATAGCTGCAAGACCAAGGACGGCACTATCGCGACTATGGAATTGTGT |
| | | GACTTTGGTTACTGGGGCCAAGGAACCCAGGTGACCGTCAGTAGCGGTGGCGGT |
| | | TCCCAAGTACAGCTGCAAGAGTCTGGAGGTGGATCTGTGCAGGCTGGGGGTAGT |
| | | CTTCGCCTGTCTTGTACCGCCAGCGGTTACGTGTCCTGTGACTACTTTCTGCCC |
| | | TCCTGGTACAGACAGGCTCCTGGTAAGGAGCGCGAGTTCGTGAGCGTCATCGAC |
| | | GGCACAGGGTCCACCAGTTACGCGGCATCCGTAAAGGGACGTTTCACCGGTTCC |
| | | CAAGATAAGGGCAAAAACATCGCTTATCTCCAGATGAACAGTTTGAAGCCTGAG |
| | | GACACAGCGATGTATTACTGTAAGGCTTCCTGCGTGAGAGGCCGCGCTATCTCT |
| | | GAATATTGGGGACAGGGCACTCAGGTGACTGTGTCTTCA |
| DR592- | 1012 | CAGGTGCAGCTCCAGGAGTCTGGGGGTGGCTCCGTCCAGGCTGGCGGAAGCCTG |
| hIL27 | | CGCCTGTCCTGTACCGCTCCGGGTTTCACAAGTAACAGTTGTGGAATGGATTGG |
| Ra_VH | | TATCGCCAGGCCCCCGGAAAGGAAGGGGAGTTCGTCTCTAGCATCTCAACTGAC |
| H5 | | GGCACTACCGGCTACGCCGACTCCGTGAAGGGCCGTTTCACCATTAGCAAGGAC |
| | | AAGGCCAAAGACACGGTGTACCTCCAGATGAACTCCCTGAAGCCCGAGGATACC |
| | | GGGATGTATAGCTGTAAGACCAAGGACGGTACTATCGCTACGATGGAGCTGTGT |
| | | GACTTCGGATACTGGGGCCAGGGGACCCAGGTGACTGTTTCCAGCGGTGGCTCC |
| | | GGTGGGTCCGGCGGTTCTGGCCAGGTGCAGCTTCCAGGAGTCCGGGGGGGGAGC |
| | | GTCCAAGCTGGCGGTTCCCCGCGCTTGAGTTGCACAGCATCTGGCTATGTGTCC |
| | | TGTGACTATTTCTTGCCATCCTGGTACAGACAAGCGCCTGGTAAGGAGAGAGAG |
| | | TTCGTGTCCGTGATTGATGGGACGGGCTCAACAAGCTATGCCGCTTCCGTTAAA |
| | | GGAAGGTTTACCGCTTCTCAAGATAAAGGGAAGAACATCGCTTATCTCCAGATG |
| | | AATAGCCTCAAACCAGAGGATACTGCTATGTATTACTGCAAGGCATCTTGTGTG |
| | | CGTGGACGTGCAATCTCCGAGTATTGGGGACAAGGCACCCAGGTTACAGTGTCT |
| | | TCA |
| DR592- | 1013 | CAGGTGCAGCTCCAGGAGTCTGGAGGTGGCTCCGTTCAGGCTGGCGGAAGCCTG |
| hIL27 | | CGGTTGAGCTGTACTGCCCCTGGATTTACCTCCAATTCTTGTGGCATGGATTGG |
| Ra_VH | | TATAGACAGGCTCCAGGGAAAGAGCGCGAGTTTGTCTCCAGCATTTCCACCGAT |
| H6 | | GGTACGACTGGTTATGCCGACAGCGTTAAGGGTAGATTTACAATCAGTAAGGAC |
| | | AAAGCTAAGGACACAGTGTATTTGCAGATGAACTCTCTGAAACCGGAGGATACA |
| | | GGTATGTATAGTTGTAAGACAAAAGATGGCACCATTGCTACTATGGAACTTTGT |
| | | GACTTCGGCTACTGGGGACAGGGCACTCAGGTAACAGTCTCTAGCGGTGGAGGC |
| | | TCACAAGTGCAGCTTCAGGAGTCTGGGGGAGGCCTCGTCCAGCCGGGAGGCTCC |
| | | TTGCGCCTGAGCTGTGCCGCTTCTGGTTTTTCATTTAGCTCCTATGCTATGAAA |
| | | TGGGTGCGCCAGGCTCCCGGCAAGGGCCTGGAGTGGGTCTCCACGATTTCCTCA |
| | | GGGGGCTCAAGCACCAACTACGCCGACAGCGTAAAGGGCCGTTTTACCATCTCT |
| | | CGGGACAACGCCAAAAACACCGTGTACCTTCAGTTGAACTCACTGAAAATCGAG |
| | | GATACTGCAATGTATTACTGTGCCAAGGCCATCGTCCCAACCGGAGCTACAATG |
| | | GAAAGGGGCCAGGGGACCCAGGTCACCGTGAGTAGC |
| DR592- | 1014 | CAGGTGCAGTTGCAGGAATCTGGTGGAGGCAGCGTGCAAGCTGGAGGCTCTCTG |
| hIL27 | | AGGTTGAGCTGCACCGCCCCTGGGTTTACAAGCAACTCATGTGGCATGGACTGG |
| Ra_VH | | TATCGGCAAGCGCCTGGGAAAGAGCGCGAATTTGTAAGCTCTATCTCTACTGAC |
| H6 | | GGCACAACCGGCTACGCAGATAGCGTGAAGGGCAGGTTCACCATCTCAAAGGAT |
| | | AAAGCCAAGGATACCGTGTACCTGCAAATGAACTCCCTTAAACCCGAGGATACC |
| | | GGCATGTATAGCTGCAAGACCAAGGACGGCACCATCGCAACGATGGAGCTGTGC |
| | | GACTTCGGTTATTGGGGACAGGGAACCCAGGTGACCGTTAGTTCCGGTGGCTCA |
| | | GGCGGTAGCGGAGGCAGTGGCCAGGTGCAGTTGCAAGAGAGCGGAGGTGGGCTG |
| | | GTGCAGCCAGGGGGCTCCCTGCGTCTGTCCCGCGCAGCCAGCGGGTTTAGCTTT |
| | | TCCAGCTATGCCATGAAATGGGTTCGGCAAGCGCCTGGGAAGGGTCTGGAATGG |
| | | GTGTCCACTATCTCCTCAGGGGGCTCCTCTACAAACTACGCAGATTCAGTGAAG |
| | | GGTCGGTTTACTATTTCTAGGGACAACGCGAAGAACACCCTGTACCTTCAGTTG |
| | | AACTCCCTCAAGATTGAGGATACTGCCATGTACTATTGGGCGAAGGCCATCGTA |
| | | CCCACCGGCGCTACAATGGAGAGGGGCCAGGGCACCCAAGTAACTGTCTCCTCA |
| DR592- | 1015 | CAAGTGCAGTTGCAGGAGAGCGGTGGGGGATCTGTCCAGGCCGGAGGCAGTCTC |
| hIL27 | | CGCCTGTCCTGCACTGCCCCTGGCTTCACCTCTAACAGTTGTGGCATGGACTGG |
| Ra_VH | | TATCGCCAGGCCCCTGGTAAAGAGCGCGAATTTGTCAGCTCAATCAGTACAGAT |
| H7 | | GGCACAACTGGTTATGCTGACAGCGTTAAGGGCCGCTTTACGATCAGCAAAGAC |
| | | AAGGCTAAAGATACTGTGTACCTCCAGATGAACAGCCTCAAGCCTGAAGACACC |
| | | GGAATGTATAGCTGTAAGACCAAGGACGGCACCATTGCGACAATGGAACTGTGC |
| | | GACTTTGGCTATTGGGGCCAAGGGACCCAGGTCACAGTCAGCAGTGGAGGGGGC |
| | | AGCCAGGTGCAGTTGCCAGGAATCAGGGGGTGGCCTTGTGCAGCCTGGGGGAGT |
| | | CTGCGCCTGTCATGTGCGGCCTCCGGGTTCACCTTCAGCTCCTACCCAATGTCT |
| | | TGGGTGAGGCAGGCACCTGGCAAGGGCCTGGAGTGGATCAGCACTATCAGCGCA |
| | | GGCGGTGACACCACTCTGTACGCAGACAGCGTGAAGGGAAGATTCACCAGTAGC |
| | | CGCGATAACGCGAAGAACACACTGTATCTCCAGCTCAATTCACTCAAGACCGAG |
| | | GACACTGCCATCTACTATTGTGCCAAACGCATTGATTGTAATTCTGGCTACTGC |
| | | TATCGGCGTAACTACTGGGGACAGGGCACCCAGGTTACCGTCAGTTCC |
| DR592- | 1016 | CAGGTTCAGCTTCAAGAGAGTGGAGGTGGGTCCGTCCAGGCCGGAGGCTCACTG |
| hIL27 | | CGGCTGTCTTGCACCGCTCCTGGTTTCACTAGCAACTCCTGCGGCATGGATTGG |
| Ra_VH | | TATAGGCAAGCCCCTGGTAAGGAACGTGAGTTTGTTTCTTCCATCTCCACAGAT |
| H7 | | GGTACTACCGGGTATGCTGACAGTGTGAAAGGCCGTTTTACCATCTCCAAGGAC |
| | | AAGGCAAAGGACACCGTGTACCTCCAGATGAACAGTCTTAAACCCGAGGACACC |
| | | GGCATGTACTCTTGTAAGACAAAGGATGGCACAATCGCCACTATGGAGCTGTGT |
| | | GATTTCGGGTACTGGGGCCAGGGCACACAGGTGACTGTGTCAAGCGGGGGAAGC |
| | | GGAGGCAGCGGTGGCTCAGGCCAGGTCCAGCTCCAGGAGTCCGGCGGTGGACTG |
| | | GTTCAACCGGGTGGCAGCCTCCGTCTGTCCTGCGCCGCGAGTGGATTCACGTTC |
| | | AGTAGCTACCCCATGTCTTGGGTCAGACAGGCTCCCGGCAAAGGTCTCGAATGG |
| | | ATCTCTACAATCTCTGCCGGTGGGGACACAACCCTGTATGCGGATAGCGTGAAA |
| | | GGGAGATTCACCAGCAGTAGGGACAACGCTAAGAACACCCTGTATCTTCAGCTG |
| | | AACAGCCTGAAAACCGAAGACACAGCGATCTATTACTGTGCAAAGAGAATTGAT |
| | | TGCAACAGCGGGTATTGCTACCGTCGCAACTATTGGGGGCAGGGAACCCAAGTC |
| | | ACTGTGTCCTCC |
| DR592- | 1017 | CAGGTGCAGCTCCAGGAATCCGGTGGAGGTTCTGTGCAGGCCGGTGGCAGCCTC |
| hIL27 | | CGTCTTAGCTGCACCGCTCCGGGGTTTACCTCCAACAGCTGTGGTATGGATTGG |
| Ra_VH | | TATCGTCAGGCCCCCGGTAAGGAGCGGGAGTTTGTGTCTTCCATTTCAACTGAT |
| H8 | | GGCACCACTGGTTACGCCGACAGCGTAAAGGGTCGTTTCACAATCAGTAAGGAT |
| | | AAGGCAAAGGACACCGTCTATTTGCAGATGAACAGGCTGAAGCCGGAAGACACA |
| | | GGGATGTACTCTTGCAAGACAAAGGACGGCACGATTGCTACGATGGAGCTGTGT |
| | | GACTTCGGCTACTGGGGCCAGGGTACACAGGTGACCGTGTCCAGCGGGGGGGGC |
| | | AGTCAGGTTCAGTTGCAGGAGTCCGGCGGTGGCCAGCGTCCAGGGGGGGGTAGC |
| | | CTGCGCCTGTCCTGTGCTGCATCCGGCTTTACCTTCTCTAGTTATCCAATGAGT |
| | | TGGGTCCGCCAGGCTCCAGGGAAAGGTCTGGAATGGATCTCCACCATTTCCGCA |
| | | GGTGGGGACACCACACTGTACGCCGATAGCGTGAAGGGCAGATTCACCAGCTCC |
| | | AGAGACAATGCCAAAAACACTTTGTACCTCCAGCTGAATAGCCTGAAGACCGAG |
| | | GATACCGCCATCTACTATTGTGCCAAGCGTATTGATTGTAATTCCGGCTATTGT |
| | | TATCGCAGAAACTACTGGGGCCAGGGCACACAGGTCACTGTATCTAGC |
| DR592- | 1018 | CAGGTGCAGCTCCAGGAGAGCGGCGGTGGCTCTGTCCAGGCTGGGGGAAGTCTG |
| hIL27 | | CGTTTGTCTTGCACTGCACCCGGATTCACCTCAAATTCCTGCGGTATGGATTGG |
| Ra_VH | | TACAGACAAGCGCCTGGCAAGGAGCGTGAATTTGTAAGCTCCATTAGCACCGAC |
| H8 | | GGGACCACTGGCTATGCTGATAGCGTGAAGGGCCGCTTCACCATTTCTAAGGAT |
| | | AAGGCTAAAGACACCGTGTACCTCCAGATGAACTCCTTGAAGCCAGAGGATACC |
| | | GGGATGTATTCTTGTAAGACCAAGGACGGCACCATCGCAACTATGGAACTCTGT |
| | | GACTTCGGTTACTGGGGGCAGGGAACCCAAGTGACAGTCTCTTCCGGGGGCTCC |
| | | GGCGGTTCTGGCGGATCTGGGCAGGTGCAACTTCAGGAATCCGGTGGAGGTAGC |
| | | GTCCAGGTGGGAGGCTCCCTGCGCCTGAGTTGTGCCGCAAGTGGCTTCACCTTC |
| | | TCATCCTACCCCATGTCTTGGGTACGCCAGGCTCCCGGCAAGGGCCTGGAGTGG |
| | | ATCTCCACCATCTCCGCCGGAGGTGACACTACCCTGTACGCCGACTCCGTGAAG |
| | | GGCCGCTTTACAAGCTCCCGTGACAACGCTAAGAATACACTGTACCTCCAGCTT |
| | | AATTCACTGAAGACCGAAGACACGGCTATCCACTATTGCGCCAAGCGCATTGAT |
| | | TGTAACAGGGGGTACTGTTACAGGAGAAACTATTGGGGACAGGGCACACAGGTG |
| | | ACTGTGTCCTCC |
| DR592- | 1019 | CAGGTTCAGGTCCAGGAGAGCGGCGGAGGCAGCGTGCAGGCTGGTGGCAGTCTG |
| hIL27 | | CGCCTGAGCTGCACAGCCCCCGGCTTCACCAGTAACAGCTGTGGAATGGACTGG |
| Ra_VH | | TATCGTCAAGCGCCTGGCAAGGAAAGGGAGTTCGTGTCTAGTATCAGCACCGAT |
| H9 | | GGCACCACAGGATACGCCGACTCCGTGAAGGGACGCTTCACTATCTCAAAGGAT |
| | | AAGGCAAAAGATACAGTGTATCTCCAGATGAACTCCCTCAAGCCTGAAGACACA |
| | | GGCATGTACTCTTGTAAGACCAAGGACGGCACTATCGCTACTATGGAGTTGTGC |
| | | GACTTCGGATACTGGGGACAAGGAACCCAGGTCACCGTATCCTCAGGGGGAGGT |
| | | TCACAGGTGCAGCTTCAGGAGTCCGGTGGCGGTTCTGTGCAGAGTGGAGGCTCC |
| | | CTGCGTTTGAGTTGCGCAGCCAGTGGGTTCACCTACTCTACTAGCAACAGCTGG |
| | | ATGGCGTGGTTCCGCCAGGCCCCCGGCAAAGAACGCGAGGGCGTTGCCGCTATT |
| | | TACACCGTGGGCGGTTCCATTTTCTACGCCGATTCCGTGCGTGGCAGGTTTACT |
| | | ATCTCTCAGGATGCCACTAAAAATATGTTCTACCTGCAAATGAACACACTGAAG |
| | | CCCGAGGACACCGCTATGTATTACTGCGCAGCCGCTTCTGGCCGCCTGAGAGGC |
| | | AAGTGGTTTTGGCCCTACGAGTACAATTACTGGGGTCAGGGCACCCAGGTGACG |
| | | GTGTGTAGC |
| DR592- | 1020 | CAGGTGCAACTCCAGGAGTCTGGAGGGGGCTCTGTCCAGGCCGGAGGCTGTCTC |
| hIL27 | | CGTCTGAGCTGCACCGCTCCCGGCTTCACCAGTAATAGGTGGGGTATGGACTGG |
| Ra_VH | | TACAGACAGGCTCCAGGCAAGGAGAGAGAATTTGTCAGCTCCATTTCTACTGAT |
| H9 | | GGGACTACGGGCTACGCCGACTCCGTGAAGGGGCGCTTTACCATGTCTAAGGAT |
| | | AAGGCTAAGGACACCGTATATTTGCAGATGAACTGCCTGAAGCCAGAAGATACT |
| | | GGCATGTATAGCTGCAAGACGAAGGATGGAACCATCGCCACTATGGAATTGTGC |
| | | GATTTCGGTTACTGGGGCCAGGGAACACAGGTGACTGTGAGTTCCGGGGGCTCA |
| | | GGCGGGTCTGGAGGCTCTGGACAGGTGCAACTCCAGGAGAGCGGAGGCGGTTCA |
| | | GTCCAGTCTGGGGGCTCCCTTCGCCTGAGTTGCGCTGCCAGTGGCTTTACCTAC |
| | | AGTACCAGCAACTCCTGGATGGCGTGGTTCAGACAGGCACCCGGAAAGGAACGC |
| | | GAAGGCGTGGCTGCCATCTACACAGTCGGGGGCTCCATCTTCTACGCTGACTCC |
| | | GTTAGAGGCCGCTTCACCATTTCTCAGGATGCCACCAAAAATATGTTCTATCTG |
| | | CAAATGAACACCTTGAAGCCCGAGGACACCGCCATGTATTACTGGGCAGGTGCC |
| | | AGCGGACGGCTTCGGGGTAAGTGGTTCTGGCCGTATGAGTATAACTACCGGGGA |
| | | CAGGGCACCCAGGTCACGGTGAGTTCT |
| DR592- | 1021 | CAAGTGCAGTTGCAGGAAAGTGGAGGGGGTTCCGTGCAGGCAGGAGGCAGTCTG |
| hIL27 | | AGGCTGAGTTGTACCGCGCCTGGCTTTACCTCAAACAGTTGCGGCATGGACTGG |
| Ra_VH | | TATAGGCAGGCCCCAGGCAAGGAGCGCGAGTTTGTCTCAAGCATCTCCACCGAT |
| H10 | | GGAACCACTGGCTACGCCGACTCCGTCAAGGGAAGGTTCACTATGTCCAAGGAT |
| | | AAGGCCAAGGACACCGTGTATCTCCAGATGAACTCATTGAAGCCAGAAGACACC |
| | | GGGATGTACTCCTGTAAAACCAAGGACGGCACGATTGCGACAATGGAACTGTGT |
| | | GATTTCGGTTACTGGGGTCAGGGAACGCAGGTGACCGTGTCTAGCGGGGGGGGT |
| | | TCCCAGGTTCAGCTCCAGGAGAGCGGTGGGGGCTCTGTGCAGGCCGGTGGAAGT |
| | | CTGCGTCTGAGCTGTCGCGCCTCTGGCTCCACATACTCCAACTATTGCCTGGGA |
| | | TGGTTTCGGCAAATCACGGGTAAAGAAAGAGAGGGCGTGGCTGTCATCAACTGG |
| | | GTGGGCGGAATGCTGTACTTCGCCGATAGTGTGAAGGGGCGCTTCACCGTTTCC |
| | | CAGGATCAAGCTAAAAACACACTTTACCTTCAGATGAACAGTCTGAAACCCGAA |
| | | GACACCGCAATGTATTACTGTGCCGCTGAGAGTGTCAGTTCCTTCTCATGCGGA |
| | | GGTTGGCTTACGAGGCCTGATCGCGTGCCGTATTGGGGCCAGGGGACCCAGGTG |
| | | ACCGTGAGTTCT |
| DR592- | 1022 | CAGGTGCAGTTGCAGGAATCCGGCGGAGGCTCCGTGCAGGCTGGGGGAAGCCTG |
| hIL27 | | AGACTTAGCTGCACTGCTCCGGGATTTACGAGCAACAGCTGTGGCATGGACTGG |
| Ra_VH | | TACAGACAGGCCCCTGGGAAGGAGCGCGAGTTCGTGTCAAGCATCAGCACTGAC |
| H10 | | GGCACAACTGGGTACGCTGATAGCGTGAAGGGCCGTTTTACCATCTCCAAGGAT |
| | | AAGGCCAAAGACACTGTCTACCTGCAAATGAACTCCCTCAAGCCTGAGGACACC |
| | | GGAATGTACTCCTGTAAGACTAAGGACGGGACCATTGCCACGATGGAGCTTTGT |
| | | GACTTCGGCTATTGGGGACAGGGAACCCAGGTCACCGTCTCTAGTGGAGGCAGG |
| | | GGCGGTAGTGGCGGGTCCGGCCAGGTTCAGCTCCAGGAGTCCGGTGGGGGATCT |
| | | GTGCAAGCAGGAGGCAGCCTGCGCCTTAGCTGCCGTGCGAGCGGCAGCACTTAC |
| | | AGCAATTATTGTCTGGGTTGGTTTCGTCAGATCACTGGTAAAGAACGGGAGGGC |
| | | GTGGCCGTCATCAACTGGGTCGGCGGTATGCTGTACTTCGCGGACAGCGTCAAA |
| | | GGCCGGTTTACCGTGTCTCAGGACCAGGCGAAGAACACATTGTACCTCCAGATG |
| | | AATAGCCTGAAGCCTGAGGATACAGCTATGTATTACTGTGCAGCGGAGTCCGTC |
| | | TCTTCATTCAGCTGTGGTGGATGGCTCACGAGACCGGATAGAGTGCCTTACTGG |
| | | GGCCAGGGCACCCAGGTGACCGTGTCCAGC |
| DR592- | 1023 | CAGGTGCAGTTGCAGGAGTCTGGTGGGGGTTCCGTTCAGGCAGGCGGTTCCCTG |
| hIL27 | | AGACTGTCCTGCACCGCCCCTGGTTTTACCTCCAACTCCTGTGGCATGGATTGG |
| Ra_VH | | TATCGCCAGGCACCAGGCAAGGAACGCGAGTTCGTATCTTCCATTAGCACTGAC |
| H11 | | GGCACAACTGGTTACGCCGATTCAGTCAAGGGGCGCTTTACAATCAGTAAGGAC |
| | | AAGGCTAAGGATACCGTTTACTTGCAGATGAATAGCCTGAAACCAGAGGACACC |
| | | GGAATGTACTCTTGCAAAACTAAAGACGGCACCATTGCAACTATGGAACTCTGT |
| | | GACTTTGGGTATTGGGGTCAAGGCACTCAAGTGACCGTCTCCTGTGGGGGGGGC |
| | | TCTCAGGTCCAGTTGCAGGAAAGCGGGGGAGGGAGCGTGCAGGCTGGAGGTAGT |
| | | CTCAGGCTGTCTTGCCGCGCCAGCGGAAGCACCTACTCAAACTACTGCCTTGGC |
| | | TGGTTTAGACAGAGCACTGGTAAGGAGCGGGAGGGAGTGGCCGTCATCAACTGG |
| | | GTCGGTGGGATGCTGTATTTCGCTGATAGCGTAAAGGGAAGATTTACGGTAAGC |
| | | CAGGATCACGCAAAGAACACCGTGACCCTCCAGATGAACTCTCTGAAGCCCGAG |
| | | GACACTGCCATGTACTATTGCGCAGCCGAATCCGTCAGTTCCTTCTCCCGCGGC |
| | | GGATGGCTGACCCGCCCCGGAAGAGTTCCATATTGGGGTCAGGGGACCCAGGTG |
| | | ACAGTTTCTTCT |
| DR592- | 1024 | CAGGTGCAACTCCAGGAGAGCGGTGGAGGTAGCGTGCAGGCTGGCGGTTCCCTG |
| hIL27 | | CGGCTGTCCTGCACCGCTCCGGGTTTTACCTCCAACTCCTGGGGGATGGATTGG |
| Ra_VH | | TACAGACAGGCCCCCGGAAAGGAGCGCGAGTTTGTGTCTTCCATTAGCACCGAC |
| H11 | | GGCACAACCGGCTATGCAGACAGTGTCAAGGGACGGTTCACAATCTCCAAGGAC |
| | | AAAGCTAAAGACACGGTGTATGTTCAGATGAACAGCTTGAAGCCTGAAGATACT |
| | | GGTATGTATTCCTGCAAAACCAAGGATGGTACTATCGCCACTATGGAACTCTGC |
| | | GATTTCGGATATTGGGGACAGGGGACCCAGGTGACGGTGTCTAGCGGGGGCTCT |
| | | GGTGGGTCTGGAGGCTCTGGTCAGGTGCAACTCCAGGAGAGCGGGGGGGGAAGC |
| | | GTTCAGGCCGGGGGCAGCCTGCGGCTGTCCTGCCGGGCTAGTGGATCTACGTAC |
| | | TCCAACTACTGCCTGGGATGGTTTCGCCAATCTACTGGAAAGGAGCGTGAGGGG |
| | | GTGGCCGTCATCAACCTGGGGGGCGGAATGTTGTATTTTGCCGATTCTGTCAAA |
| | | GGAAGGTTCACTGTGTCTCAGGACCATGCCAAGAACACCGTCACCCTCCAGATG |
| | | AACTCTTTGAAGCCGGAAGATACCGCTATGTATTACTGCGCGGCAGAAAGCGTG |
| | | AGTAGCTTCTCTTGCGGTGGGTGGCTGACACGTCCGGGTAGGGTGCCGTACTGG |
| | | GGCCAGGGAACTCAGGTAACCGTCTCATCC |
| DR592- | 1025 | CAGGTCCAACTCCAGGAATCCGGGGGGGGCTCCGTACAGGCAGGTGGCAGTCTG |
| hIL27 | | AGGCTTTCCTGCACTGCCCCGGTTTCACCAGCCAACAGCTGTGGGATGGACTGG |
| Ra_VH | | TATCGCCAGGCTCCCGGCAAAGAGCGGGAGTTCGTCAGCTCTATCTCAACCGAC |
| H12 | | GGCACAACCGGGTATGCCGACTCCGTTAAAGGCCGGTTCACCATCAGCAAAGAC |
| | | AAGGCCAAGGATACAGTCTACCTCCAGATGAACAGCCTGAAGCCTGAGGATACC |
| | | GGAATGTATAGTTGCAAAACTAAGGATGGCACTATCGCCACTATGGAGTTGTGC |
| | | GATTTCGGATACTGGGGACAGGGCACCCAGGTTACCGTGTCATCTGGAGGGGGC |
| | | TCCCAGGTGCAGCTGCAAGAATCCGGCGGAGGCAGTGTCCAGGCTGGCGAATCC |
| | | CTCCGCTTGAGCTGTCGTGCCAGCGGTAGTACCTACTCCAATTACTGCCTGGGT |
| | | TGGTTTCGCCAGATCACCGGCAAGGAACGTGAAGGCGTGGCCGTCATCAACTGG |
| | | GTAGGGGGTATGCTCTACTTCGCTGACTCCGTGAAGGGACGCTTCACGGTGAGT |
| | | CAGGACCAGGCCTAAAAACACCGTGTACCTGAAATGAACTCCTTGAAGCCCGAG |
| | | GACACCGCTATGTATTACTGCGCCACAGAGTCTGTTTCTTCCTTCTCATGCGGA |
| | | GGCTGGCTGACTCGTCCAGACCGCGTGCCCTACTGGGGCCAGGGGACCCAGGTG |
| | | ACCGTGTCCTCA |
| DR592- | 1026 | CAAGTTCAGTTGCAGGAGAGTGGAGGCGGTTCTGTCCAGGCTGGGGGTAGTTTG |
| hIL27 | | CGGTTGAGTTGCACCGCGCCTGGGTTCACCTCCAACAGCTGTGGTATGGATTGG |
| Ra_VH | | TATCGCCAGGCCCCCGGCAAGGAGCGCGAGTTCGTGAGTTCCATCAGCACCGAC |
| H12 | | GGCACCACTGGTTACGCAGACTCAGTGAAGGGAAGATTCACCATCAGCAAGGAC |
| | | AAAGCCAAGGACACTGTGTATCTTCAGATGAATAGCCTCAAGCCGGAGGATACC |
| | | GGCATGTATTCCTGCAAGACCAAGGACGGCACCATCGCTACGATGGAGTTGTGT |
| | | GATTTCGGGTACTGGGGACAGGGAACCCAGGTAACTGTGAGTAGCGGAGGTAGG |
| | | GGGGGAAGCGGCGGGTCAGGGCAGGTACAGCTTCCAGGAGTCCGGGGGGGGTCC |
| | | GTTCAGGCCGGTGAGTCCTTGCGCCTGAGCTGCCGCGCCAGCGGTTCCACCTAC |
| | | AGTAACTATTGTCTGGGCTGGTTTCGTCAGATCACAGGCAAGGAGCGCGAGGGC |
| | | GTTGGTGTCATTAACTGGGTTGGCGGAATGCTGTATTTTGCCGACAGCGTGAAG |
| | | GGTCGTTTCACGGTATCCCAAGACCAAGCAAAGAACACTGTCTATCTTGAAATG |
| | | AACAGTCTGAAACCAGAAGACACCGCCATGTACTATTGTGCCACCGAGTCCGTA |
| | | TCCAGTTTCTCCTGCGGGGGATGGCTGACCAGACCTGACCGTGTGCCATACTGG |
| | | GGACAGGGGACACAGGTGACTGTGTCCAGC |
| DR592- | 1027 | CAGGTGCAGCTCCAGGAGTCCGGGGGAGGCTCTGTGCAGGCCGGGGGTTCCCTG |
| hIL27 | | AGACTTTCATGTACTGCACCAGGCTTTACCTCAAATTCTTGCGGGATGGACTGG |
| Ra_VH | | TACAGGCAGGCCCCCGGTAAGGAGCGGGAGTTTGTGTCTTCCATTTCCACCGAT |
| H13 | | GGTACAACCGGCTACGCGGACTCTGTGAAGGGCCGCTTTACCATTTCTAAGGAC |
| | | AAGGCTAAAGACACCGTCTATCTCCAGATGAACTCTTTGAAGCCTGAAGACACA |
| | | GGCATGTATAGCTGCAAGACAAAGGATGGCACCATTGCCACGATGGAGTTGTGC |
| | | GACTTTGGCTATTGGGGTCAGGGCACCCAGGTCACTGTGTCTTCCGGGGGGGGC |
| | | TCTCAGGTCCAACTCCAGGAGAGCGGGGGAGGGTCTGTTCAGGCTGGTGGCTCA |
| | | CTGCGCCTCAGTTGCGTGGCCTCTGGATACGTGTCCTGTGACTACTTTCTGCCC |
| | | TCTTGGTACAGACAGGCCCCAGGCAAAGAGCGCGAGTTCGTGAGCATCATTGAC |
| | | GGTACTGGCTCTACAAGCTACGCGGCCTCCGTCAAGGGCCGTTTCACTGCCTCC |
| | | CAAGACAGAGGTAAAAACATCGCTTATGTCCAGATGAACAGCCTGAAGCCCGAG |
| | | GATACGGCTATGTACTATTGCAAGGCCAGCTGTGTACGTGGACGCACTATCTCC |
| | | GAGTACTGGGGCCAGGGGACACAAGTGACGGTGTCCTCC |
| DR592- | 1028 | CAGGTGCAGCTTCAAGAGAGCGGAGGCGGGTCTGTGCAGGCTGGTGGGTCCCTC |
| hIL27 | | CGCCTGTCATGCACTGCCCCCGGCTTCACTTCTAACAGCTGGGGCATGGACTGG |
| Ra_VH | | TATCGCCAGGCCCCAGGCAAAGAGCGCGAGTTTGTATCATCCATGTCCACCGAC |
| H13 | | GGCACGACTGGCTACGCCGACTCCGTGAAGGGCCGGTTCACCATCAGCAAGGAC |
| | | AAAGCCAAGGATACCGTGTACCTCCAGATGAACTCCCTCAAACCTGAAGACACT |
| | | GGGATGTACTCATGCAAGACAAAAAGACGGTACGATGCCACTATGGAGCTGTGT |
| | | GATTTCGGATACTGGGGACAGGGAACTCAGGTGACCGTTTCCTCTGGAGGCAGT |
| | | GGAGGGAGCGGAGGCTCCGGCCAGGTGCAGCTGCAAGAGAGCGGTGGGGGTAGC |
| | | GTGCAAGCCGGAGGCTCCCTCCGTCTTTCTTGTGTCGCGAGCGGCTACGTGAGC |
| | | TGTGATTACTTTCTGCCCAGCTGGTATCGTCAAGCGCCAGGCAAAGAGCGCGAA |
| | | TTTGTGTCTATTATCGACGGCACCGGCAGCACGAGCTACGCAGCCTCCGTGAAG |
| | | GGACGTTTCACTGCTTCACAGGATCGCGGCAAAAACATTGCTTACCTCCAGATG |
| | | AACTCCCTGAAGCCTGAGGACACAGCAATGTATTACTGTAAGGCCTCCTGTGTG |
| | | CGTGGACGCACCATCTCAGAGTACTGGGGACAGGGAACCCAGGTGACCGTATCT |
| | | TCC |
| DR592- | 1029 | CAGGTTCAGGTTCAGGAGTCCGGGGGTGGCTCCGTCCAAGCTGGTGGCTCACTG |
| hIL27 | | CGCCTGTCCTGCACCGCCCCTGGCTTCACCTCCAATTCCTGCGGAATGGACTGG |
| Ra_VH | | TATCGCCAGGCCCCAGGCAAAGAACGCGAGTTCGTCAGTAGCATCTCTACTGAT |
| H14 | | GGAACCACAGGCTACGCGGACAGCGTGAAGGGGCGTTTCACTATCTCCAAGGAC |
| | | AAGGCCAAGGACACCGTGTACCTTCAAATGAACTCCCTGAAGCCCGAGGATACT |
| | | GGAATGTACTCCTGTAAGACCAAGGACGGGACTATCGCCACTATGGAGCTGTGT |
| | | GACTTCGGATATTGGGGCCAGGGCACGCAGGTGACCGTCAGCTCTGGGGGGGGG |
| | | TCCCAGGTCCAGCTTCAGGAAAGTGGAGGGGGGTCCGTGCAGGCCGGAGGTTCC |
| | | CTCCGCCTGTCATGTGTCGCAAGCGGCTACGTGTCTTGCGACTACTTCCTGCCC |
| | | TCCTGGTATCGCCAGGCTCCGGGCAAGGAGAGAGAGTTCGTAAGCATCATTGAT |
| | | GGCACCGGCTCTACTTCTTACGCCGCTAGTGTGAAGGGCCGCTTCACAGCGTCC |
| | | CAGGATAAGGGTAAAAACATCGCTTACTTGCAGATGAATAGCCTGAAGCCTGAG |
| | | GACACGGCAATGTATTACTGTAAGGCCTCCTGTGTTAGAGGCCGCGCTATTTCT |
| | | GAATACTGGGGACAGGGCACTCAGGTGACAGTCTCCCCC |
| DR592- | 1030 | CAGGTGCAGCTTCAGGAGTCCGGTGGGGGCTCTGTGCAGGCTGGAGGTTCCCTT |
| hIL27 | | CGCCTGTCATGTACTGCCCCTGGGTTCACTTCTAACTCGTGTGGTATGGACTGG |
| Ra_VH | | TATCGTCAGGCCCCTGGGAAGGAGCGTGAGTTCGTCAGGTCCATCTCTACTGAT |
| H14 | | GGCACTACCGGCTATGCCGATTCTGTGAAGGGGCGTTTTACCATCAGCAAGGAC |
| | | AAGGCCAAGGATACCGTGTACCTGCAAATGAACAGCCTGAAGCCTGAGGACACT |
| | | GGCATGTATTCTTGTAAAACCAAAGACGGCACCATCGCCACAATGGAGCTGTGT |
| | | GACTTTGGCTACTGGGGCCAGGGCACTCAAGTGACCGTGTCCTCAGGGGGGTCT |
| | | GGCGGATCTGGGGGAAGCGGACAGGTGCAGCTTCAGGAGAGCGGAGGTGGCTCT |
| | | GTCCAGGCCGGTGGGTCCCTGAGACTGTCTTGCGTGGCCAGTGGGTACGTGAGC |
| | | TGCGATTACTTCCTCCCCAGTTGGTATAGGCAGGCCCCTGGAAAGGAACGTGAG |
| | | TTCGTAAGCATCATTGACGGCACCGGCTCTACTTCTTATGCGGCGTCCGTAAAG |
| | | GGCCGCTTCACTGCTTCCCAGGACAAGGGTAAGAATATCGCCTACCTTCAGATG |
| | | AACAGCCTGAAACCCGAAGACACGGCTATGTATTACTGCAAGGCTTCCTGCGTA |
| | | AGAGGGAGAGCAATCTCCGAGTATTGGGGCCAGGGCACCCAGGTGACTGTGTCT |
| | | TCA |
| DR592- | 1031 | CAGGTGCAGCTCCAGGAGAGCGGGGGGGGCTCCGTACAAGCGGGGGGTTCCCTC |
| hIL27 | | CGTCTGTCTTGTACCGCCCCAGGCTTCACCAGTAACTCCTGTGGCATGGACTGG |
| Ra_VH | | TACAGACAGGCTCCTGGCAAAGAAAGAGAGTTCGTATCCAGCATGTCCACCGAC |
| H15 | | GGCACCACAGGCTACGCGGATTCAGTTAAGGGCAGATTTACGATGTCCAAGGAT |
| | | AAGGCTAAAGATACTGTGTACCTCCAGATGAATAGCCTGAAGCCGGAAGACACC |
| | | GGCATGTATAGCTGTAAGACTAAGGATGGCACCATTGCCACAATGGAGCTGTGC |
| | | GACTTCGGCTACTGGGGTCAGGGAACACAGGTGACGGTGTCCTCTGGTGGCGGT |
| | | AGCCAGGTTCAGTTGCAGGAATCTGGAGGTGGCTCTGTGCAGGCCGGTGGATCT |
| | | CTGCGTCTGTCTTGCGTGGCCTCCGGCTACGTGAGTTGCGACTATTTCCTTCCC |
| | | AGCTGGTATCGCCAGGCTCCAGGCAAGGAACGCGAGTTTGTGTCCATTATCGAC |
| | | GGAACAGGCAGCACATCCTACGCAGCTAGTGTTAAGGGTAGATTCACTGCGAGC |
| | | CAGGACAAGGGCAAAAACATCGCTTATCTTCAGATGAATACCCTCAAACCTGAG |
| | | GACACCGCCATGTATTACTGCAAGGCCAGCTGTGTGCGTGGCCGCGCTATCTCT |
| | | GAATATTGGGGCCAGGGCACCCAGGTGACAGTTTCCTCT |
| DR592- | 1032 | CAGGTGCAGCTCCAGGAGAGCGGGGGAGGCTCAGTGCAGGCCGGGGGTTCATTG |
| hIL27 | | AGGCTGTCCTGCACCGCCCCAGGGTTTACTAGCAACTCTTGTGGGATGGATTGG |
| Ra_VH | | TATCGTCAGGCCCCTGGGAAGGAACGTGAGTTCGTTTCTAGTATCTCAACCGAT |
| H15 | | GGTACAACCGGCTATGCAGACTCCGTAAAGGGCCGGTTCACGATGTCCAAAGAT |
| | | AAGGCTAAGGACACTGTTTATCTGCAAATGAACTCCCTGAAGCCTGAGGACACC |
| | | GGCATGTACTCCTGTAAGACTAAGGACGGCACCATCGCTACGATGGAGTTGTGC |
| | | GACTTCGGATATTGGGGTCAGGGAACCCAGGTTACTGTGTCTAGCGGGGGCAGC |
| | | GGTGGCAGGGGGGGATCTGGCCAGGTGCAGTTGCAGGAATCCGGGGGGGGTTCA |
| | | GTGCAGGCAGGAGGCTCTCTTCGGTTGTCCTGCGTCGCGAGTGGCTATGTGTCT |
| | | TGTGATTACTTCCCGCCATCCTGGTATCGTCAGGCCCCAGGGAAGGAACGTGAG |
| | | TTCGTGTCCATCATTGATGGCACTGGCAGTACTTCCTACGCCGCTTCCGTGAAG |
| | | GGTAGGTTTACAGCGAGTCAGGATAAGGGTAAGAACATCGCCTATCTCCAGATG |
| | | AACACCCTGAAACCGGAGGACACAGCCATGTACTATTGCAAGGCCTCTTGTGTG |
| | | CGTGGCCGCGCAATTAGCGAGTACTGGGGACAGGGTACACAGGTTACCGTCTCC |
| | | TCC |
| DR592- | 1033 | CAGGTCCAGCTCCAGGAGAGTGGTGGGGGCAGCGTGCAGGCCGGAGGCTCTCTT |
| hIL27 | | AGACTCTCTTGCACCGCCCCCGGCTTCACATCCAACTCCTGTGGCATGGACTGG |
| Ra_VH | | TATCGCCAGGCTCCCGGCAAGGAGAGGGAATTTGTGTCCAGCATTTCTACCGAC |
| H16 | | GGAACCACAGGCTACGCTGACAGCGTTAAGGGACGTTTCACTATCAGCAAGGAT |
| | | AAGGCCAAGGACACCGTATACCTCCAGATGAACTCCCTCAAACCCGAGGACACC |
| | | GGCATGTATAGCTGTAAAACCAAGGATGGAACCATCGCGACTATGGAGCTGTGC |
| | | GATTTCGGCTACTGGGGCCAGGGCACTCAAGTGACTGTTTCTAGCGGCGGGGGC |
| | | AGCCAGGTGCAGCTTCAGGAGTCTGGCGGAGGCAGCGTCCAAGCTGGAGGTAGC |
| | | CTGCGGCTCTCCTGTCGCGCTTCTGGCTCTACGTACAGTAATTATTGCCTGGGT |
| | | TGGTTCAGACAGATCACCGGCAAAGAACGCGAGGGTGTTGCAGTCATCAACTGG |
| | | GTTGGGGGAATGCTGTATTTTGCCGACAGTGTGAAGGGCAGGTTCACGGTCAGC |
| | | CAAGACCAGGCCAAGAATACCGTCTACCTCCAGATGAACTCTTTGAAACCCGAA |
| | | GACACCGCCATGTACTATTGTGCTGCGGAGTCAGCTTCATCTTTCTCTTGCGGA |
| | | GGCTGGCTGACCCGTCCAGACCGCGTACCTTACTGGGGCCAGGGGACCCAGGTG |
| | | ACCGTCTCCTCC |
| DR592- | 1034 | CAAGTCCAGCTGCAAGAGAGTGGAGGGGGCAGCGTTCAGGCCGGAGGCTCTCTG |
| hIL27 | | CGTCTTTCATGCACCGCGCCCGGTTTCACTTCCAACTCCTGCGGCATGGACTGG |
| Ra_VH | | TATCGGCAGGCCCCAGGCAAGGAAAGGGAGTTCGTAAGGTCCATCTCCACTGAT |
| H16 | | GGAACAACCGGCTATGCCGATTCCGTCAAGGGCCGCTTCACCATCAGCAAGGAT |
| | | AAGGCAAAAGACACTGTCTATCTGCAAATGAACTCTCTGAAGCCCGAAGACACC |
| | | GGAATGTACTCTTGTAAAACTAAGGACGGCACCATTGCCACTATGGAGCTGTGT |
| | | GATTTCGGGTACTGGGGCCAGGGCACCCAGGTGACGGTCTCTAGCGGGGGCTCA |
| | | GGGGGCTCTGGTGGCTCTGGTCAGGTGCAGCTCCAGGAGTCCGGTGGCGGAAGC |
| | | GTGCAGGCTGGAGGTAGTCCGCGCCTGTCTTGTCGTGCCAGCGGCAGCACATAT |
| | | AGCAATTACTGCCTTGGCTGGTTTAGACAGATTACTGGTAAGGAGCGGGAGGGG |
| | | GTGGCCGTAATAAATTGGGTTGGCGGGATGCTCTATTTTGCAGACTCAGTCAAG |
| | | GGCCGCTTCACAGTGTCCCAGGATCAGGCCAAGAACACCGTGTATCTCCAGATG |
| | | AACTCCCTGAAGCCGGAAGACACCGCCATGTACTATTGTGCCGCAGAGTCCGCT |
| | | TCCTCTTTCTCTTGGGGCGGATGGCTGACACGTCCTGATCGTGTGCCCTATTGG |
| | | GGACAGGGCACACAGGTCACCGTGTCCTCC |
| DR592- | 1035 | CAGGTTCAGCTGCAAGAGAGCGGCGGTGGCTCTGTGCAGGCTGGTGGCAGCTTG |
| hIL27 | | AGACTGTCCTGTACTGCGCCTGGCTTCACTTCCAATTCTTGCGGCATGGACTGG |
| Ra_VH | | TATAGACAAGCTCCGGGAAAAGAACGCGAATTTGTGTCTTCCATTAGCACCGAC |
| H17 | | GGAACTACAGGCTATGCTGACAGCGTGAAGGGACGCTTTACCATCTCCAAGGAC |
| | | AAGGCGAAGGATACCGTCTATTTGCAGATGAACAGTCTGAAACCAGAAGACACC |
| | | GGCATGTATTCCTGCAAAACCAAAGATGGCACAATCGCCACTATGGAACTCTGT |
| | | GATTTTGGCTACTGGGGCCAAGGGACCCAAGTTACCGTGTCTTCCGGGGGAGGC |
| | | AGCCAAGTCCAGTTGCAAGAGTCAGGTGGCGGTCTGGTCCAGCCCGGAGGCTCT |
| | | CTGAGGCTGAGTTGTGCCGCTTCCGGTTTCACATTCTCCCTGAGCGGTATGTCC |
| | | TGGGTACGCCAGGCTCCCGGCAAGGGCCTCGAATGGGTGTCCGCGATCAGCTCC |
| | | GGCGGGGCCTCCACTTATTACACAGACAGCGTTAAGGGGCGCTTCACAATCAGC |
| | | CGCGATAACGCAAAGAATATGTTGTACTTGCAACTCAATTCTCTCAAGACAGAA |
| | | GACACCGCGATGTATTACTGTGCTAAAGGGGGTTCCGGCTATGGCGATGCCTCT |
| | | CGTATGACTTCACCCGGTAGCCAGGGAACCCAGGTGACCGTCTCTAGC |
| DR592- | 1036 | CAGGTGCAGCTCCAAGAGTCTGGGGGGGGTTCTGTGCAGGCAGGGGGGTCCTTG |
| hIL27 | | AGACTCTCTTGCACCGCCCCAGGCTTTACCAGCAATTCGTGTGGAATGGATTGG |
| Ra_VH | | TACAGGCAAGCGCCCGGTAAAGAACGCGAGTTCGTCAGCTCTATTAGCACAGAG |
| H17 | | GGAACCACTGGCTATGCTGACTCAGTTAAGGGCCGGTTCACCATCAGTAAGGAT |
| | | AAAGCCAAGGACACCGTGTACCTTCAGATGAACTCTCTGAAGCCCGAGGACACT |
| | | GGCATGTATAGCTGTAAGACAAAAGACGGAACAATCGCGACTATGGAGTTGTGT |
| | | GACTTCGGATACTGGGGTCAGGGAACGCAGGTGACCGTGTCCAGCGGCGGTTCT |
| | | GGGGGTAGCGGGGGCAGCGGACAAGTTCAGCTCCAGGAAAGCGGAGGGGGGCTC |
| | | GTGCAGCCTGGGGGCTCCCTCCGCCTGTCCTGTGCAGCCTCAGGCTTCACGTTT |
| | | TCACTGTCTGGCATGAGCTGGGTTCGCCAGGCTCCCGGCAAAGGACTCGAATGG |
| | | GTGTCCGCCATCTCTTCCGGCGGAGCCTCCACCTATTACACAGATTCCGTCAAA |
| | | GGACGCTTCACTATCTCACGTGATAACGCGAAGAATATGTTGTACCTTCAGTTG |
| | | AACTCCCTGAAAACCGAGGATACAGCCATGTATTACTGGGCTAAGGGGGGAAGC |
| | | GGATACGGAGACGCCTCCCGCATGACATCCCCTGGTTCCCAGGGGACACAGGTG |
| | | ACCGTGTCCTCA |
| DR592- | 1037 | CAGGTCCAGCTCCAGGAATCCGGGGGGGGTCCGTGCCAGGCGGGTGGCTCCCTT |
| hIL27 | | AGGCTCTCCTGTACCGCCCCCGGCTTCACCAGCAACTCCTGTGGGATGGATTGG |
| Ra_VH | | TATCGGCAAGCACCGGGCAAGGAGCGCGAGTTTGTGTCCTCTATCAGCACCGAC |
| H18 | | GGCACTACCGGCTACGCAGACAGCGTAAAGGGTCGTTTCACAATCTCCAAGGAT |
| | | AAGGCAAAGGACACAGTGTATGTTCAGATGAACAGCCTGAAGCCCGAGGATACT |
| | | GGCATGTATTCCTGCAAGACCAAAGACGGAACCATCGCCACTATGGAACTCTGC |
| | | GATTTCGGCTACTGGGGGCAAGGCACCCAGGTGACGGTGTCTAGCGGCGGTGGC |
| | | AGCCAGGTGCAGTTGCAGGAGTCTGGAGGGGGCTCCGTGCAGGCTGGAGGCAGC |
| | | CTGCGCCTGTCTTGCGTTGCTTCCGGGTATGTCTCTTGCGATTACTTCCTGCCT |
| | | AGCTGGTACAGACAGGCCCCTGGGAAGGAGCGCGAGTTCGTTTCAATCATTGAT |
| | | GGGACGGGCTCCACCTCCTATGCTGCATCCGTTAAGGGGCGCTTTACAGCCTCT |
| | | CAGGATAAGGGAAAAAATATCGCCTACCTCCAGATGAACTCTCTGAAGCCTGAG |
| | | GACACTGCCATGTACTATTGCAAGGCCTCATGTGTTCGCGGGGGGGGTATTAGT |
| | | GAGTACTGGGGGCAGGGCACCCAGGTGACTGTGTCCAGC |
| DR592- | 1038 | CAAGTTCAGCTCCAGGAGAGCGGAGGTGGGTCAGTCCAGGCTGGTGGCAGTCTG |
| hIL27 | | CGTCTGTCCTGCACTGCCCCCGGTTTCACCTCCAACAGCTGCGGCATGGATTGG |
| Ra_VH | | TACAGGCAGGCACCTGGCAAAGAAAGGGAGTTCGTGTCCTCTATTTCCACTGAC |
| H18 | | GGAACGACCGGCTACGCCGACAGTTGTGAAGGGAAGGTCACAATCTCCAAGGAC |
| | | AAGGCCAAGGACACCGTGTATCTTCAGATGAACTCACTGAAGCCCGAAGACACC |
| | | GGGATGTATAGCTGCAAGACAAAGGACGGGACTATCGCTACTATGGAGCTGTGT |
| | | GATTTCGGTTACTGGGGGCAAGGCACTCAGGTGACCGTTAGCTCCGGCGGTAGG |
| | | GGCGGATCAGGAGGCTCTGGGCAAGTGCAGCTTCAAGAGTCTGGGGGTGGCTCC |
| | | GTACAAGCGGGCGGAAGCCTGAGACTGAGTTGCGTGGCTTCCGGCTACGTTAGC |
| | | TGCGACTACTTTCTGCCAAGCTGGTATCGCCAAGCTCCCGGCAAGGAGGGGGAG |
| | | TTCGTGTCAATTATCGACGGCACTGGTTCTACCTCTTACGCCGGTTCTGTAAAG |
| | | GGACGCTTTACCGCCAGCCAGGATAAGGGCAAAAACATCGCATACCTTCAGATG |
| | | AACTCTCTGAAGCCAGAGGACACAGCAATGTATTACTGTAAGGCTTCCTGCGTT |
| | | CGCGGTCGCGGTATCTCTGAATATTGGGGCCAGGGGACCCAGGTAACTGTGTCT |
| | | TCT |
| DR592- | 1039 | CAGGTGCAACTTCAGGAATCTGGAGGGGGATCAGTCCAGGCCGGGGGTAGTCTG |
| hIL27 | | AGATTGTCTTGCACCGCTCCCGGATTTACGTCAAACTCTTGCGGTATGGACTGG |
| Ra_VH | | TATCGCCAGGCTCCCGGCAAGGAACGTGAGTTCGTGTCCAGCATTAGTACCGAT |
| H19 | | GGCACCACGGGCTATGCTGACAGTGTGAAGGGTCGGTTTACCATGTCCAAGGAC |
| | | AAGGCCAAGGACACCGTGTATGTTCAGATGAACAGCCTGAAGCCCGAAGACACC |
| | | GGCATGTATTCCTGCAAGACCAAGGATGGCACCATCGGTACGATGGAACTGTGC |
| | | GATTTCGGTTACTGGGGTCAGGGAACCCAGGTCACCGTGTCTAGCGGAGGCGGT |
| | | TCTCAAGTGCAGCTCCAGGAATCTGGCGGGGGCTCCGTGCAGGCTGGGGGGTCC |
| | | CTGCGCCTGTCCTGTCGGGCGAGCGGTTCCACCTACTCCAATTATTGTCTGGGC |
| | | TGGTTTAGACAGATCACAGGCAAGGAAAGAGAGGGCGTGGCCGTTATCAACTGG |
| | | GTCGGAGGTATGCTTTACTTCGCCGACAGCGTCAAGGGCAGGTTCACCGTCTCT |
| | | CAGGACCAAGCTAAGAATACCGTGTACCTTCAGATGAACTCCCTGAAGCCTGAG |
| | | GATACCGCGATGTATTACTGTGCAGCTGAGTCCGTCAGCTCTTTCTCTTGTGGA |
| | | GGTTGGTTGACTAGGCCTGATCGGGTGCCATACTGGGGCCAGGGGACACAGGTC |
| | | ACCGTATCTAGC |
| DR592- | 1040 | CAGGTACAGTTGCAGGAAAGCGGCGGTGGATCTGTCCAGGCCGGGGGTAGCCTC |
| hIL27 | | CGCCTGTCCTGCACAGCCCCTGGGTTCACCAGCAACTCATGTGGCATGGACTGG |
| Ra_VH | | TATCGCCAGGCTCCGGGTAAAGAGCGTGAGTTCGTGTCCTCTATCTCCACAGAT |
| H19 | | GGAACGACAGGCTATGCAGACAGCGTCAAAGGGAGGTTTACAATCTCCAAGGAC |
| | | AAGGCCAAGGATACGGTTTACCTTCAGATGAACTCTCTGAAGCCCGAGGACACC |
| | | GGCATGTACTCCTGTAAGACTAAGGACGGAACCATCGCCACTATGGAACTCTGG |
| | | GATTTCGGCTATTGGGGACAGGGAACCCAGGTGACGGTGTCCAGGGGAGGCTCA |
| | | GGAGGGTCCGGTGGCAGCGGACAGGTCCAACTCCAGGAGTCTGGTGGAGGCTCC |
| | | GTCCAGGCCGGAGGTTCTCTGAGACTGTCTTGTCGGGCATCTGGATCTACTTAC |
| | | AGCAACTACTGCCTGGGATGGTTCAGACAAATCACAGGAAAGGAGCGCGAAGGT |
| | | GTGGCCGTGATTAACTGGGTGGGCGGTATGCTGTATTTCGCAGACTCCGTCAAG |
| | | GGCCGCTTCACCGTGTCTCAGGATCAGGCTAAAAACACTGTTTATCTCCAGATG |
| | | AACTCCCTCAAGCCTGAGGATACTGCCATGTATTACTGTGCCGCAGAGTCCGTA |
| | | AGCTCCTTCTCTTGGGGGGCTGGCCTGACTCGCCCCGACCGTGTACCGTACTGG |
| | | GGCCAGGGAACACAGGTCACAGTGTCCTCT |
| DR592- | 1041 | CAGGTGCAGCTCCAGGAATCTGGCGGTGGCTCCGTGCAGGCCGGTGGCTCCCTC |
| hIL27 | | AGACTGTCCTGCACAGCGCCAGGCTTCACGTCCAACTGTTGCGGCATGGATTGG |
| Ra_VH | | TATCGGCAAGCACCGGGCAAGGAAAGAGAGTTCGTTTCTTCCATCTCCACGGAC |
| H20 | | GGCACGACAGGCTATGCGGACTCAGTCAAAGGCCGTTTCACTATTTCTAAGGAT |
| | | AAAGCCAAGGATACAGTCTACGTTCAGATGAACTCCTTGAAACCAGAGGACACC |
| | | GGAATGTATAGTTGCAAGACTAAGGACGGCACAATCGCCACTATGGAACTGTGC |
| | | GACTTCGGGTACTGGGGCCAGGGCACACAGGTCACTGTTAGCTCCGGTGGCGGT |
| | | TCACAGGTCCAGCTCCAGGAGAGCGGCGGGGGGCTGGTTCAGCCGGGAGGGAGC |
| | | CTGCGCCTGTCCTGTGCCGCTTCCGGGTTCACGTTTTCTTCCTACCCTATGTCC |
| | | TGGGTTCGCCAGGCCCCTGGTAAGGGTCTTGAATGGGTCTCTACAATTTCTAGT |
| | | GGTGGCGACACAACGCTGTATGCCGACTCCGTCAAGGGTCGCTTTACCTCAAGC |
| | | AGGGATAATGCTAAGAACACCCTCTATTTGCAGTTGAACTCTCTTAAAACTGAG |
| | | GATACCGCAATGTATTACTGTGCAAAACGTATCGACTGTAATAGCGGTTATTGC |
| | | TATAAGCGTAGCTACTGGGGTCAGGGCACCCAGGTGACGGTGTCTAGT |
| DR592- | 1042 | CAGGTGCAACTCCAGGAATCCGGGGGGGGCAGCGTTCAGGCGGGAGGCTCTCTC |
| hIL27 | | CGTCTGTCCTGCACCGCCCCCGGCTTCACAAGTAATTCGTGGGGCATGGACTGG |
| Ra_VH | | TATCGTCAGGCCCCTGGCAAAGAACGGGAGTTCGTAAGCTCCATCTCAACCGAT |
| H20 | | GGCACAACCGGCTATGCAGACTCCGTGAAGGGGCGCTTCACCATGTCAAAGGAC |
| | | AAGGCCAAAGATACAGTGTACCTTCAGATGAACAGCCTTAAACCGGAGGATACA |
| | | GGCATGTATTCTTGCAAGACCAAGGACGGCACCATCGCAACAATGGAGCTGTGC |
| | | GACTTCGGATACTGGGGTCAAGGCACCCAGGTGACTGTGTCATCTGGGGGTAGC |
| | | GGAGGGAGCGGAGGCAGTGGTCAGGTGCAGCTTCAGGAGAGCGGTGGAGGGCTC |
| | | GTGCAACCCGGGGGTAGCCTGCGCCTGTCCTGCGCAGCTTCTGGGTTTACGTTT |
| | | TCTTCCTACCCTATGAGCTGGGTGAGGCAGGCCCCTGGCAAGGGGCTGGAGTGG |
| | | GTCTCAACCATTTCCAGCGGTGGGGATACCACACTCTACGCCGATTCCGTCAAG |
| | | GGACGCTTCACCAGTTCCCGCGATAATGCCAAGAACACCCTGTACCTTCAGCTG |
| | | AACAGCCTGAAGACTGAGGACACTGCCATGTACTATTGTGCGAAGCGGATTGAC |
| | | TGCAATTCTGGGTACTGTTATAAGCGTTCTTACTGGGGGCAAGGAACCCAGGTG |
| | | ACCGTGTCCAGT |
| DR592- | 1043 | CAAGTGCAGCTTCAAGAAAGCGGGGGGGGTTCCGTGCAGGCAGGGGGGAGCCTG |
| hIL27 | | CGCTTGAGTTGCACCGCTCCGGGCTTTACAAGTAACAGCTGCGGGATGGATTGG |
| Ra_VH | | TATCGCCAGGCACCTGGAAAGGAGCGCGAGTTCGTGTCCTCTATTTCCACGGAC |
| H21 | | GGGACTACCGGGTATGCCGATTCCGTGAAGGGCCGGTTTACTATGTCCAAGGAC |
| | | AAAGCCAAGGACACCGTTTACCTTCAGATGAACAGCCTCAAGCCTGAAGATACC |
| | | GGAATGTATAGTTGTAAGACCAAGGACGGCACCATCGCCACGATGGAGCTGTGT |
| | | GATTTCGGATACTGGGGTCAGGGCACACAGGTTACTGTGTCCAGTGGAGGCGGT |
| | | AGCCAGGTGCAGTTGCAGGAATCTGGCGGGGGCTTGGTCCAGCCTGGGGGCTCA |
| | | CTGCGCCTCAGCTGCGCGGCCTCCGGCTTCACGTTTAGCCTGAGTAGCATGTCC |
| | | TGGGTTCGGCAAGCACCGGGTAAGGGCCTGGAGTGGGTCTCCGCTATTTCCAGC |
| | | GGCGGTGCTAGTACCTATTACACCGACTCTGTGAAAGGCCGCTTCACGATTTCC |
| | | CGCGACAACGCAAAGAACATGGTTTATCTCCAGCTGAACTCACTGAAGACAGAG |
| | | GACACAGCAATGTATTACTGTGCCAAGGGAGGTTCCGGGTACGGGGATGCGTCA |
| | | CGTATGACATCCCCTGGTTCACAGGGCACCCAGGTGACGGTGAGCAGC |
| DR592- | 1044 | CAAGTCCAGCTCCAGGAGTCCGGGGGGGGTTCCGTCCAGGCTGGCGGTAGCCTG |
| hIL27 | | AGGCTGTCCTGTACCGCCCCCGGTTTCACATCTAACTCCTGCGGCATGGACTGG |
| Ra_VH | | TATCGCCAGGCTCCCGGAAAAGAACGCGAGTTCGTGAGTTCCATGTCCACAGAC |
| H21 | | GGCACCACTGGCTATGCAGACAGTGTGAAGGGAAGGTTCACCATCTCTAAGGAT |
| | | AAAGCTAAGGACACCGTTTACTTGCAGATGAACTCTCTCAAGCCAGAGGACACC |
| | | GGAATGTACTCCTGTAAGACCAAAGATGGGACCATCGCGACAATGGAGCTGTGT |
| | | GACTTTGGATACTGGGGACAAGGGACCCAGGTGACTGTTTCATCCGGCGGTTCT |
| | | GGCGGGTCTGGTGGGTCCGGTCAGGTGCAGCTCCAGGAGTCCGGGGGGGGACTG |
| | | GTGCAGCCTGGGGGATCACTGCGCCTCAGCTGCGCTGCCTCAGGTTTCACATTT |
| | | AGTCTTAGCTCTATGAGCTGGGTGCGCCAGGCACCGGGAAAGGGGCTGGAATGG |
| | | GTCAGCGCAATCAGCTCAGGCGGGGCTTCTACGTATTACACGGATAGCGTGAAA |
| | | GGGCGCTTCACCATCAGCCGTGACAACGCCAAGAACATGGTGTACCTTCAGCTG |
| | | AACTCACTGAAGACGGAAGATACTGCCATGTACTATTGTGCCAAGGGTGGATCC |
| | | GGGTATGGTGATGCCTCCCGTATGACCTCACCTGGAAGCCAGGGTACACAGGTC |
| | | ACCGTCTCCTCC |
| DR592- | 1045 | CAGGTGCAGTTGCAGGAGAGCGGCGGGGGCAGGGTGCAAGCCGGTGGCAGCCTG |
| hIL27 | | CGGCTGTCTTGTACCGCCCCCGGTTTTACTAGCAATTCTTGCGGCATGGATTGG |
| Ra_VH | | TATCGCCAGGCCCCAGGCAAGGAAAGAGAGTTCGTGTCCTCTATCAGCACTGAC |
| H22 | | GGCACAACCGGCTACGCCGATTCAGTGAAGGGCCGCTTCACCATCTCAAAGGAT |
| | | AAGGCCAAGGACACCGTGTACCTTCAGATGAACTCTCTTAAACCGGAGGACACT |
| | | GGTATGTACTCTTGCAAGACCAAAGACGGGACCATCGCGACTATGGAGCTGTGC |
| | | GATTTCGGGTACTGGGGCCAGGGAACACAGGTCACTGTCTCTTCCGGCGGAGGT |
| | | TCCCAGGTTCAGTTGCAGGAGAGCGGTGGGGGTAGCGTTCAGGCTGGCGGAAGC |
| | | CTGCGGTTGAGTTGTCGCGCATCTGGCTCTACCTATAGCAACTATTGCCTGGGA |
| | | TGGTTCAGACAGACAACCGGCAAGGAGCGCGAGGGTGTGGCCGTCATCAACTGG |
| | | GTCGGGGGTATGCTGTATTTTGCTGATTCCGTGAAGGGCCGTTTCACCGTGAGG |
| | | CAGGACCAGGCTAAGAACACAGTTTATCTCCAGATGAACAGCCTGAAACCCGAG |
| | | GATACTGCCATGTACTATTGTGCGGCAGAATCCGTGTCCTCTTTTAGCTGTGGT |
| | | GGCTGGCTCACACGCCCTGACCGCGTCCCGTACTGGGGTCAGGGAACCCAAGTG |
| | | ACCGTGAGTTCA |
| DR592- | 1046 | CAAGTGCAGCTTCAGGAGTCTGGCGGGGGCTCCGTACAGGCTGGGGGCAGCCTT |
| hIL27 | | AGATTGTCATGCACTGCCCCTGGATTCACAAGCAACAGGTGTGGCATGGATTGG |
| Ra_VH | | TATCGCCAAGCACCTGGGAAGGAGCGGGAGTTCGTCTCCAGCATTTCTACTGAT |
| H22 | | GGTACGACCGGCTACGCTGACAGTGTGAAGGGACGCTTCACTATCTCCAAGGAC |
| | | AAGGCCAAAGATACCGTGTATTTGCAGATGAACAGTCTCAAGCCGGAGGACACC |
| | | GGAATGTATTCCTGCAAGACTAAGGACGGGACTATCGCCACTATGGAACTTTGC |
| | | GACTTTGGCTATTGGGGCCAGGGCACCCAGGTGACCGTAAGTTCGGGGGGTTCC |
| | | GGCGGAAGCGGCGGTAGCGGCCAGGTTCAACTTCAGGAGTCCGGGGGAGGCTCT |
| | | GTGCAGGCCGGGGGCTCTCTGAGACTGTCCTGCCGGGCCTCCGGGTCCACATAC |
| | | TCTAACTACTGCCTGGGATGGTTCCGTCAGACTACAGGCAAGGAGCGCGAGGGT |
| | | GTTGCAGTCATCAACTGGGTAGGGGGAATGCTGTATTTCGCCGATTCAGTCAAA |
| | | GGACGCTTCACTGTGTCCCAAGACCAGGCCAAGAACACAGTCTATCTTCAGATG |
| | | AACAGCCTCAAGCCCGAGGATACCGCTATGTACTATTGCGCAGCTGAGAGCGTG |
| | | TCCAGCTTCTCTTGCGGAGGGTGGCCGACCAGGCCGGATCGGGTGGCCCATTGG |
| | | GGTCAAGGCACACAGGTGACAGTTTCAAGT |
| DR592- | 1047 | CAGGTGCAGCTCCAGGAGAGTGGAGGTGGGTCCGTGCAGGCGGGAGGTAGCCTG |
| hIL27 | | CGGCTGTCCTGTACTGCGCCCGGCTTTACCAGTAACAGGTGGGGCATGGACTGG |
| Ra_VH | | TATAGACAGGCCCCCGGAAAGGAACGCGAGTTTGTCAGCAGTATCTCTACTGAT |
| H23 | | GGCACCACTGGATATGCGGACTCCGTGAAGGGTCGGTTCACCATTAGCAAGGAC |
| | | AAGGCAAAGGATACCGTGTATTTGCAGATGAACAGTCTCAAACCAGAGGACACA |
| | | GGCATGTATAGTTGCAAAACCAAGGACGGGACCATCGCCACTATGGAACTCTGT |
| | | GATTTTGGGTATTGGGGCCAGGGGACTCAGGTGACCGTCAGCTCCGGGGGGGGT |
| | | TCCCAAGTTCAGTTGCAAGAGTCTGGAGGGGGATCAGTGCAGGCCGGGGGGTCT |
| | | CTGCGGCTTAGCTGCCGTGCTTCTCGCAGCCCTTATGGGAACTACTGTCTTGGC |
| | | TGGTTCAGGCAGAGTACAGGTAAGGAACGCGAAGGGGTGGCCGTCATCAACTGG |
| | | GTGGGCGGAATGCTCTATTTCGCTGACTCTGTGAAGGGCCGTTTCACAGTGTCC |
| | | CAGGACCATGCTAAGAACACTGTCACCCTCCAGATGAACAGCCTGAAGCCAGAG |
| | | GACACCGCTATGTATTACTGCGCAGCCGAGTCTGTGTCCAGTTTCTCATGCGGT |
| | | GGGTGGCTGACCAGACCTGATCGCGTTCCATACTGGGGACAAGGGACTCAGGTC |
| | | ACAGTGTCCTCT |
| DR592- | 1048 | CAGGTGCAGCTCCAGGAGTCCGGGGGTGGCTCCGTTCAGGCGGGAGGCTCTTTG |
| hIL27 | | CGTTTGTCCTGCACCGCACCCGGTTTCACAAGTAACTCCTGCGGCATGGACTGG |
| Ra_VH | | TATCGTCAGGCTCCAGGCAAGGAGCGCGAGTTCGTGAGTTCTATCTCCACAGAC |
| H23 | | GGCACCACGGGCTACGCCGACAGTGTTAAGGGTCGCTTTACTATCTCAAAGGAT |
| | | AAGGCGAAGGACACCGTATACCTTCAGATGAACTCCCTCAAGCCTGAAGACACC |
| | | GGAATGTATAGCTGTAAGACCAAGGACGGCACCATCGCCACAATGGAGCTGTGC |
| | | GACTTTGGATACTGGGGGCAGGGAACACAGGTGACAGTATCCAGCGGGGGCTCC |
| | | GGCGGTTCCGGCGGTTCCGGCCAGGTCCAGTTGCAGGAGTCTGGTGGCGGTAGG |
| | | GTCCAGGCTGGGGGCAGCCTGCGGCTGTCCTGTCGCGCATCAAGAAGCCCTTAC |
| | | GGCAATTATTGTTTGGGCTGGTTCCGCCAATCAACTGGTAAGGAGAGAGAAGGA |
| | | GTCGCTGTCATTAACTGGGTGGGCGGTATGCTTTATTTTGCCGATAGCGTGAAG |
| | | GGGCGCTTCACGGTGTCTCAGGACCATGCCAAGAATACCGTTACGTTGCAGATG |
| | | AACAGCCTGAAACCCGAAGACACCGCAATGTATTACTGTGCCGCTGAGTCCGTA |
| | | TCCTCTTTCTCCTGTGGAGGCTGGCTGACACGCCCCGACCGCGTGCCTTATTGG |
| | | GGCCAAGGGACCCAGGTTACCGTGTCCTCC |
| DR592- | 1049 | CAGGTGCAGTTGCAGGAAAGTGGTGGAGGCAGCGTGCAGGCAGGAGGCTCTCTG |
| hIL27 | | CGCCTGAGCTGCACAGCGCCTGGCTTTACCTCCAACAGCTGTGGAATGGACTGG |
| Ra_VH | | TATCGCCAGGCTCCCGGTAAGGAGCGGGAATTTGTCAGCTCCATTTCCACCGAC |
| H24 | | GGCACCACTGGCTACGCCGATTCCGTCAAGGGCAGGTTCACAATCTCAAAGGAC |
| | | AAAGCCAAGGACACAGTGTATCTCCAGATGAACTCCCTGAAGCCTGAGGACACA |
| | | GGTATGTATAGCTGCAAGACTAAGGACGGCACAATCGCCACGATGGAATTGTGC |
| | | GACTTCGGATATTGGGGACAAGGTACTCAGGTGACCGTCTCCTCTGGAGGGGGC |
| | | TCCCAGGTGCAGCTCCAGGAGTCTGGCGGTGGCCTGGTGCAGCCCGGTGGGTCT |
| | | CTGCGGCTGAGTTGTGCTGCGTCTGGATTCACCTTTTCCCACAGCGGGATGTCA |
| | | TGGGTGAGGCAGGCACCCGGCAAGGGCGTGGAGTGGGTGAGCACAATTAACTCC |
| | | GGGGGGCTTCCACGTACTATACCGATAGCGTGAAGGGCCAGGTTTACCATCTCA |
| | | CGCGACAACGCGAAGAACATGGTGTATCTTCAGCTGAATAGTTTGAAGACCGAG |
| | | GACACCGCTATGTATTACTGCGCCAAAGGCGGTTCTGGGTACGGGGACGCCTCC |
| | | CGCATGACAAGCCCTGGGTCTCAGGGCACACAGGTGACCGTGTCTTCT |
| DR592- | 1050 | CAAGTTCAGCTTCAGGAATCAGGTGGCGGTAGCGTGCAGGCTGGGGGCTCCTTG |
| hIL27 | | AGATTGTCCTGCACGGCCCCAGGATTCACCAGCAACTCTTGGGGGATGGATTGG |
| Ra_VH | | TATCGCCAGGCTCCGGGGAAGGAGAGGGAGTTGGTAAGCTCCATTTCAACCGAT |
| H24 | | GGGACCACTGGATACGCTGACTCCGTTAAGGGTAGGTTCACGATCAGTAAGGAC |
| | | AAGGCCAAGGATACAGTGTATGTTCAAATGAACTCACTGAAGCCCGAGGACACC |
| | | GGGATGTACTCCTGTAAGACCAAAGATGGCACTATCGCAACGATGGAGCTGTGC |
| | | GACTTCGGCTATTGGGGCCAGGGAACCCAGGTTACTGTGTCCAGCGGAGGCAGG |
| | | GGCGGTTCTGGAGGCTCTGGACAGGTCCAACTCCAGGAATCTGGGGGCGGTCTC |
| | | GTGCAGCCAGGTGGGTCACTGCGCTTGTCATGTGCGGCCTCCGGTTTCACCTTC |
| | | AGCCATTCTGGAATGAGCTGGGTGCGTCAAGCACCGGGCAAAGGCCTCGAATGG |
| | | GTCTCTACTATTAACAGCGGAGGTGCATCTACCTACTATACAGACAGCGTTAAG |
| | | GGGAGGTTCACCATCTCTCGCGATAATGCTAAAAACATGCTGTATTTGCAGCTT |
| | | AATTCACTGAAGACGGAGGACACGGCCATGTACTATTGTGCCAAGGGTGGGTCC |
| | | GGGTACGGTGACGCCTCTCGCATGACCTCCCCAGGAAGTCAGGGCACCCAGGTG |
| | | ACCGTGTCATCC |
| DR593- | 1051 | CAGGTGCAGGTCCAGGAATCTGGCGGTGGCAGCGTGCAGGCCGGTGGCTCCCTG |
| hIL27 | | AGGCTGTCCTGTGCTGCCTCTGGATACCCATACTCCAACGGGTACATGGGCTGG |
| Ra_VH | | TTCCGTCAGGCCCCTGGAAAGGAACGTGAGGGCGTGGCCACTATTTACACCGGC |
| H1 | | GACGGTCGTACTTATTACGCCGACTCCGTGAAAGGCCGGTTCACCATTTCTAGG |
| | | GATAACGCTAAGAACACAGTTGACCTCCAGATGTCTAGCCTGAAACCTGAGGAT |
| | | ACCGCCATGTATTACTGCGCTGCCAGGGCTGCCCCACTCTACAGCTCTGGTTCC |
| | | CCCCTGACACGCGCCAGGTATAACGTGTGGGGACAGGGGACCCAGGTCACAGTG |
| | | AGTTCTGGCGGGGGTTCTCAGGTGCAACTCCAGGAATCTGGCGGAGGCCTGGTC |
| | | CAGCCTGGTGGCTCCCTTCGCCTGAGCTGTGCAGCCTCTGGCTTCACATTCAGC |
| | | TCCTATCCTATGTCCTGGGTGCGCCAGGCCCCTGGGAAGGGCCTGGAGTGGATC |
| | | TCCACCATCTCTGCGGGTGGCGACACTACACTCTACGCCGACAGCGTGAAGGGC |
| | | CGCTTTACCTCCAGTCGCGATAACGCTAAGAATACACTGTACCTCCAGCTTAAC |
| | | AGCCTGAAGACAGAGGACGCTGCAATCTATTACTGTGCCAAGAGGATCGACTGC |
| | | AACAGCGGATATTGCTACCGTAGGAATTATTGGGGCCAGGGAACCCAGGTTACC |
| | | GTATCATCC |
| DR593- | 1052 | CAAGTTCAGTTGCAGGAATCCGGTGGGGGTAGCGTGCAGGCTGGTGGCAGCCTG |
| hIL27 | | CGTCTGAGCTGCGCTGCATCCGGGTATCCGTACAGCAACGGTTACATGGGATGG |
| Ra_VH | | TTCCGCCAAGCTCCTGGGAAGGAGCGCGAAGGCGTGGCGACCATTTACACCGGA |
| H1 | | GACGGACGCACTTATTACGCCGACTCAGTGAAGGGCCGGTTTACGATCTCTCGG |
| | | GACAACGCTAAGAATACCGTGGACTTGCAGATGAGTTCCCTGAAGCCTGAGGAC |
| | | ACCGCAATGTATTACTGTGCCGCACGCGCTGCGCCCCTCTACAGCTCTGGTTCT |
| | | CCCCTGACCCGTGCCCGTTACAATGTATGGGGACAAGGCACCCAGGTGACCGTG |
| | | AGTAGCGGGGGAAGCGGAGGCAGCGGGGGATCTGGCCAGGTGCAGCTCCAGGAA |
| | | AGCGGCGGGGGCCTGGTCCAGCCCGGTGGCAGCCTGCGCCTGTCCTGTGCTGCC |
| | | AGTGGATTCACCTTTTCTTCCTATCCTATGTCATGGGTCCGCCAGGCACCTGGC |
| | | AAGGGCCTGGAGTGGATTTCCACTATCTCAGGTGGCGGAGACACAACTCTCTAC |
| | | GCGGACTCTGTAAAGGGTCGGTTTACGTCTTCCCGCGACAACGCCAAGAATACC |
| | | CTGTACCTTCAGTTGAACTCCCTTAAAACCGAAGATGCAGCTATCTACTATTGT |
| | | GCGAAGCGTATTGATTGCAACTCCGGTTACTGCTACCGGCGTAACTACTGGGGT |
| | | CAAGGCACACAGGTCACAGTCTCCTCC |
| DR593- | 1053 | CAAGTCCAGCTCCAAGAGAGTGGGGGGGGTTCCGTGCAGGCTGGAGGCTCCCTC |
| hIL27 | | AGATTGAGTTGTGCGGCTAGTGGCTATCCCTATAGCAACGGCTACATGGGCTGG |
| Ra_VH | | TTCCGCCAGGCCCCCGGCAAGGAAAGGGAGGGTGTTGCAACCATGTACACAGGA |
| H2 | | GATGGCCGCACTTACTATGCAGACTCCGTTAAGGGTCGTTTCACCATTTCCCGC |
| | | GATAACGCTAAGAATACAGTGGATCTTCAGATGTCCAGCCTCAAACCAGAGGAT |
| | | ACTGCAATGTATTACTGCGCAGCGCGGGCCGCGCCCCTGTACTCTTCCGGGTCT |
| | | CCACTGACCCGCGCTCGCTACAACGTGTGGGGCCAAGGCACGCAGGTCACAGTT |
| | | TCTTCCGGCGGGGGCAGCCAGGTCCAGGTCCAGGAAAGTGGCGGGGGCCTGGTT |
| | | CAGCCCGGGGGTTCCCTGCGCCTCAGCTGTGCTGCCTCTGGGTTTACCTTCAGC |
| | | CTGTCAGGCATGAGCTGGGTGCGGCAGGCTCCTGGCAAGGGTCTGGAATGGGTT |
| | | TCCGCCATTTCAAGCGGGGGGGCCTTCTACCTATACACAGACTGCGTGAAGGGC |
| | | CGTTTCACGATCAGTCGCGACAACGCGAAAAACATTCTCTATCTCCAGCTGAAC |
| | | AGCTTGAAGACCGAGGACACTGCGATGTATTACTGTGCCAAAGGGGGATCAGGA |
| | | TACGGCGATGCGTCTCGCATGACTTCCCCAGGTTCACAGGGCACTCAGGTTACC |
| | | GTCTCTTCG |
| DR593- | 1054 | CAGGTACAGCTTCAAGAAAGGGGGGGGGGAAGCGTGCAAGCTGGGGGGAGCCTT |
| hIL27 | | AGGCTGAGTTGCGCGGCCTCAGGTTACCCCTATAGCAACGGATATATGGGATGG |
| Ra_VH | | TTCCGGCAGGCACCGGGGAAGGAACGCGAGGGAGTGGCCACCATTTACACCGGG |
| H2 | | GACGGGAGGACCTACTATGCCGACTCTGTGAAGGGCCGTTTCACTATCTCTAGG |
| | | GATAACGCGAAGAACACAGTGGACTTGCAGATGTCATCTCTCAAGCCGGAGGAC |
| | | ACTGCCATGTATTACTGTGCGGCTCGTGCCGCTCCTCTGTACAGCTCCGGGAGT |
| | | CCACTGACCCGCGCAAGGTACAACGTGTGGGGTCAGGGAACTCAGGTGACCGTT |
| | | TCATCCGGGGGCAGCGGTGGCTCCGGTGGCTCTGGTCAGGTTCAGCTCCAGGAA |
| | | AGCGGGGGGGGGCTGGTGCAGCCCGGAGGCTCCCTGCGTCTTTCTTGCGCTGCC |
| | | AGTGGCTTTACCTTTTCCCTGAGTGGAATGAGCTGGGTGAGGCAAGCGCCAGGC |
| | | AAGGGACTGGAATGGGTGAGCGCAATCAGTTCAGGGGGGGCCAGTACTTATTAC |
| | | ACTGACTCCGTCAAGGGTCGGTTTACCATCTCTCGGGATAACGCTAAGAACATC |
| | | CTTTATTTGCAGCTCAACTCCTTGAAGACGGAAGACACCGCTATGTATTACTGC |
| | | GCTAAAGGAGGGAGTGGATACGGAGACGCATCTCGCATGACCAGTCCAGGTTCC |
| | | CAGGGAACCCAGGTGACTGTGTCAAGC |
| DR593- | 1055 | CAGGTCCAGGTTCAGGAAAGCGGCGGTGGGTGTGTCCAGGCAGGTGGCTCTCTG |
| hIL27 | | AGACTGTCCTGTGCTGCCAGCGGGTATCCGTACTCAAATGGCTACATGGGCTGG |
| Ra_VH | | TTCCGCCAGGCCCCTGGAAAGGAGCGCGAGGGCGTGGCCACGATCTATACCGGG |
| H3 | | GACGGACGCACCTATTACGCTGACAGTGTCAAAGGTAGATTCACTATCAGCCGG |
| | | GACAACGCGAAGAACACCGTTGACCTTCAGATGTCAAGCCTGAAGCCTGAGGAC |
| | | ACTGCCATGTATTACTGCGCTGCAAGGGCTGCCCCTCTTTACAGCTCCGGCTCA |
| | | CCCCTTACAAGAGCCCGGTACAACGTCTGGGGCCAGGGAACTCAGGTGACCGTT |
| | | AGCTCCGGCGGGGGCTCCCAGGTGCAGCTCCAGGAGTCCGGTGGCGGAAGTGTT |
| | | CAGGCGGGCGGAAGTCTGCGCCTGTCTTGCGTGGCCTCTGGATACGTCTCCTGT |
| | | GACTATTTCTTGCCCAGCTGGTATCGCCAGGCTCCGGGAAAAGAGAGAGAGTTC |
| | | GTGAGCATCATTGACGGCACTGGCTCCACCAGCTACGCAGCCCCCGTTAAGGGT |
| | | CGCTTTACCGCCTCAGAGGATAAGGGTAAGAACATCGCATACTTGCAGATGAAC |
| | | AGTCTCAAGCCCGAGGATACCGCTATGTATTACTGCAAGGCCAGGTGCGTCCGG |
| | | GGTCGCGCTGTCTCCGAATACTGGGGACAGGGCACCCAGGTGACCGTATCTAGT |
| DR593- | 1056 | CAGGTTCAGCTCCAGGAGAGTGGCGGAGGGAGTGTGCAAGCTGGCGGTTCTTTG |
| hIL27 | | CGCCTCTCTTGTGCTGCCTCCGGTTATCCCTACAGTAACGGCTATATGGGCTGG |
| Ra_VH | | TTTCGCCAAGCACCAGGGAAGGAACGGGAGGGGGTCGCCACCATTTATACTGGC |
| H3 | | GATGGCCGCACCTATTACGCCGACTCCGTGAAAGGTCGCTTCACCATCAGTCGG |
| | | GACAATGCAAAGAACACTGTAGACTTGCAAATGTCTTCCCTCAAGCCTGAGGAT |
| | | ACTGCTATGTACTATTGCGCTGCAAGGGCAGCTCCCCTGTACTCTAGTGGCTCT |
| | | CCTCTGACCCGCGCTCGGTACAACGTCTGGGGTCAGGGAACCCAGGTTACCGTC |
| | | TCTAGCGGGGGCTCAGGTGGCAGCGGGGGTTCCGGCCAGGTCCAACTCCAGGAG |
| | | TCCGGCGGTGGCTCCGTTCAAGCCGGAGGCAGTCTGAGACTGAGCTGCGTGGCG |
| | | TCCGGTTACGTCTCCTGTGACTACTTCCTGCCTAGCTGGTATCGGCAAGCGCCC |
| | | GGTAAGGAAAGAGAGTTCGTGAGTATCATTGATGGGACCGGCTCCACCTCCTAC |
| | | GCCGCATCTGTTAAGGGGCGCTTCACAGCTTCCGAAGACAAGGGAAAGAACATC |
| | | GCTTATCTTCAGATGAACTCTCTCAAACCTGAAGACACGGCTATGTACTATTGC |
| | | AAGGCCTCCTGTGTACGGGGTCGCGCCGTGTCCGAGTACTGGGGGCAGGGGACA |
| | | CAGGTTACCGTATCCAGC |
| DR593- | 1057 | CAGGTGCAGCTGCAAGAGAGCGGGGGAGGCAGCGTGCAAGCTGGAGGCTCTCTC |
| hIL27 | | CGGCTTTCATGCGCTGCCAGCGGCTATCCTTATAGCAACGGCTACATGGGCTGG |
| Ra_VH | | TTCCGCCAAGCGCCGGGCAAGGAGCGCGAGGGGGTGGCGACGATTTATACGGGG |
| H4 | | GACGGCAGGACTTATTACGCAGACTCCGTCAAAGGGCGCTTCACCATCTCCCGC |
| | | GACAACGCCAAAAATACCGTCGATCTTCAGATGTCAAGCCTCAAGCCCGAGGAT |
| | | ACAGCCATGTACTATTGCGCAGCCGGCGCCGCACCGCTCTATTGGTCAGGATCT |
| | | CCACTCACACGCGCCCGGTACAATGTGTGGGGTCAAGGAACCCAGGTGACCGTA |
| | | TCCAGCGGGGGGGGTTCTCAAGTCCAGCTTCAGGAGTCCGGCGGAGGCCTTGTG |
| | | CAGCCCGGAGAATCTCTGAGACTGTCCTGTACTGCCAGGGGGTTCACCTTTTCC |
| | | AACTACGCGATGAGCTGGGTACGGCAAGCGCCCGGAAAGGGTCTGGAATGGGTC |
| | | AGCGGAATCAATGTTGCCTACGGCATCACTTCTTACGCCGACAGTGTTAAGGGC |
| | | CGCTTCACTATTAGTCGTGACAACACCAAAAACACTCTGTATCTCCAGCTCAAC |
| | | AGCCTGAAAACAGAGGATACTGCGATTTACTATTGTGTCAAACACTCTGGCACC |
| | | ACTATCCCAAGGGGGTTCATCTCCTACACTAAGCGCGGTCAGGGAACCCAGGTG |
| | | ACCGTGTCATCC |
| DR593- | 1058 | CAGGTGCAGTTGCAGGAGAGTGGTGGCGGAAGCGTGCAAGCTGGCGGAAGCCTG |
| hIL27 | | AGACTGAGCTGCGCTGCCGAGCGGCTACCCTTACTCAAATGGCTACATGGGGGG |
| Ra_VH | | TTCCGTCAGGCCCCCGGCAAGGAGCGTGAGGGGGTGGCCACGATCTACACTGGA |
| H4 | | GATGGCCGCACTTATTACGCCGATTCTGTTAAGGGGAGATTCACAATCAGCCGC |
| | | GACAATGCTAAGAACACCGTGGACCCTCAGATGTCCAGCCTCAAACCGGAAGAC |
| | | ACTGCAATGTATTACTGTGCGGCCCGTGCCGCGCCGCTTTACTCTTCAGGGTCA |
| | | CCTCTGACCCGCGCTCGGTACAATGTATGGGGCCAGGGTACACAGGTGACCGTG |
| | | TCTAGCGGAGGCAGCGGTGGCTCAGGAGGTTCTGGGCAAGTCCAGCTCCAAGAG |
| | | TCAGGTGGCGGTCTGGTGCAGCCAGGTGAAAGCCTGCGTCTTTCCTGCACTGCC |
| | | AGCGGCTTTACCTTCTCCAACTATGCCATGTCGTGGGTGCGCCAAGCTCCGGGG |
| | | AAGGGCCTGGAATGGGTATCCGGCATTAACGTGGCCTATGGGATCACAAGTTAC |
| | | GCTGACTCTGTGAAGGGCAGATTTACTATTAGCAGAGACAACACCAAGAATACC |
| | | TTGTACTTGCAACTGAACTCCCTCAAGACCGAGGATACAGCCATCTACTATTGC |
| | | GTGAAACACAGCGGCACTACAATCCCGCGCGGCTTTATTAGTTATACGAAGAGG |
| | | GGACAAGGTACGCAGGTGACCGTGTCCAGC |
| DR593- | 1059 | CAGGTGCAGTTGCAGGAGTCCGGCGGGGGCAGCGTTCAGGCTGGGGGCTCTCTC |
| hIL27 | | AGACTGTCCTGTGCCGCGAGCGGCTATCCATACAGTAACGGATACATGGGCTGG |
| Ra_VH | | TTCCGCCAAGCGCCCGGCAAGGAGAGGGAGGGTGTCGCCACTATCTACAGGGGG |
| H5 | | GATGGCCGGACTTATTACGCGGATAGCGTGAAAGGTCGTTTTACCATCTCCCGC |
| | | GACAACGCGAAGAACACCGTGGATTTGCAGATGTCTAGCCTCAAACCCGAGGAC |
| | | ACGGCTATGTACTATTGCGCAGCCCGTGCCGCACCTTTGTATTCCTCTGGTTCA |
| | | CCCCTCACTAGGGCCAGATATAACGTGTGGGGACAGGGGACCCAGGTGACTGTG |
| | | AGTTCTGGGGGTGGCTCCCAGGTGCAGCTCCAGGAGAGCGGAGGTGGCTCCGTT |
| | | CAGGGGGGAGGCTCCCTGAGGCTGTCTTGCACCGCCTCTGGATACGTTTCCTGC |
| | | GACTATTTCCCCCCTCCTGGTACAGACAGGCACCCGGAAAGGAACCGCGAGTTT |
| | | GTGAGCGTCATCGACGGAACAGGGAGCACCTCCTATGCGGCCTCAGTGAAGGGC |
| | | AGATTCACAGCTTCCCAAGACAAGGGTAAGAACATCGCATACCTTCAGATGAAC |
| | | TCCCTGAAACCCGAAGATACCGCCATGTATTACTGCAAAGCATCTTGCGTTCGG |
| | | GGAAGGGCAATCAGCGAATACTGGGGCCAGGGCACGCAGGTGACCGTGTCCTCT |
| DR593- | 1060 | CAGGTGCAGGTTCAAGAGAGTGGTGGAGGGTCCGTCCAGGCCGGGGGTTCTCTG |
| hIL27 | | AGGTTGTCCTGCGCAGCCAGCGGTTACCCCTACTCCAACGGCCTATATGGGGGG |
| Ra_VH | | TTCCGTCAGGCCCCAGGCAAGGAACGCGAAGGGGTCGCAACAATCTACACAGGT |
| H5 | | GATGGCCGCACTTATTACGCTGATAGCGTGAAAGGTCGGTTCACCATTTCTCGG |
| | | GACAACGCCAAGAACACCGTAGATTTGCAGATGTCTTCCTTGAAACCCGAGGAC |
| | | ACTGCCATGTATTACTGCGCTGCACGGGCTGCCCCACTCTATAGTTCCGGCTCC |
| | | CCACTCACACGCGCCAGATACAACGTGTGGGGGCAAGGGACACAGGTCACTGTG |
| | | TCTAGTGGGGGCTCCGGTGGCAGCGGAGGTAGCGGCCAGGTTCAGCTTCAGGAA |
| | | TCTGGCGGAGGCTCCGTGCAAGCCGGTGGGAGCCTCAGACTGTCTTGTACTGCC |
| | | AGCGGGTACGTAAGCTGCGACTACTTCGTGCCTTCCTGGTATCGCCAAGGTCCA |
| | | GGGAAGGAACGTGAGTTCGTATCTGTGATCGACGGAACAGGCTCTACATCTTAT |
| | | GCAGCTTCAGTGAAGGGGAGATTCACAGCCAGCCAGGACAAGGGTAAAAACATT |
| | | GGTTATCTCCAGATGAACTCACTCAAGCCGGAGGACACCGCTATGTATTACTGT |
| | | AAAGCATCCTGCGTGAGAGGACGGGCTATCAGCGAATACTGGGGGCAGGGAACC |
| | | CAGGTGACCGTGAGCAGC |
| DR593- | 1061 | CAGGTGCAGTTGCAGGAGTCCGGGGGAGGTTCCGTCCAGGCCGGGGGAAGTCTG |
| hIL27 | | CGCTTGTCATGCGCTGCCAGCGGCTATCCCTACTCCAACGGCTATATGGGCTGG |
| Ra_VH | | TTTAGGCAGGCCCCTGGGAAGGAACGCGAAGGGGTCGCGACGATGTACACGGGC |
| H6 | | GATGGGAGGACCTATTACGCAGATTCCGTAAAGGGCAGATTTACCATCCCCCGT |
| | | GATAACGCCAAGAATACCGTTGACCTCCAGATGTCCTGTCTGAAACCCGAGGAT |
| | | ACCGCCATGTACTATTGCGCCGCTCGCGCCGCTCCCCTCTACAGCTCTGGATCA |
| | | CCACTGACCCGCGCTAGGTATAACGTGTGGGGTCAGGGGACACAGGTGACCGTC |
| | | AGCTCTGGGGGAGGCTCCCAGGTTCAGCTCCAGGAGTCTGGAGGGGGCCTTGTG |
| | | CAGCCCGGCGGGTCCCTCCGTCTGAGTTGTGCTGCCTCAGGCTTTAGTTTCTCT |
| | | TCCTACGCTATGAAATGGGTCCGTCAAGCGCCCGGCAAAGGCCTGGAGTGGGTC |
| | | TCTACCATTAGCTCCGGGGGTTCCAGCACGAATTATGCTGACTCCGTCAAAGGC |
| | | AGGTTCACTATCAGCCGCGACAACGCCAAGAATACCCTGTACTTGCAGCTCAAC |
| | | AGCCTGAAGATCGAGGACACCGCGATGTATTACTGCGCTAAAGCCATCGTGCCC |
| | | ACTGGAGCGACGATGGAACGCGGACAGGGCACTCAGGTCACCGTGTCCAGC |
| DR593- | 1062 | CAAGTGCAGGTGCAAGAATCAGGCGGAGGCAGTGTCCAAGCTGGGGGTAGTCTG |
| hIL27 | | CGCCTGAGCTGCGCCGCATCAGGCTACCCATACTCCTAACGGCTATATGGGGGG |
| Ra_VH | | TTTAGGCAAGCCCCCGGCAAGGAGCGCGAAGGCGTGGCGACCATCTACACTGGT |
| H6 | | GATGGCCGCACTTACTATGCGGACTCTGTGAAAGGACGGTTCACAATTTCTCGT |
| | | GACAATGCTAAGAACACTGTGGATCTTCAGATGAGTTCCCTGAAGCCCGAAGAC |
| | | ACGGCGATGTATTACTGTGCTGCCAGAGCCGCTCCGCTGTACTCCAGCGGCAGG |
| | | CCCCTCACCCGCGCCCGTTATAACGTCTGGGGGCAGGGCACCCAGGTCACTGTT |
| | | TCCTCCGGTGGCAGTGGAGGCTCTGGGGGCAGCGGACAAGTACAACTCCAGGAG |
| | | TCAGGCGGGGGCCTGGTTCAGCCAGGGGGTTCCCTGCGCCTGAGCTGTGCCGCT |
| | | TCTGGCTTTTCATTCTCTTCCTATGCCATGAAGTGGCGGGGCCAGGCCCGTGGG |
| | | AAGGGACTGGAGTGGGTGAGCACCATTTCAAGTGGAGGTAGCTCCACCAACTAC |
| | | GCGGATTCCGTCAAAGGCCGCTTCACCATTTCCAGGGACAACGCTAAAAACACC |
| | | CTGTATCTTCAGCTCAACTCCGTGAAGATCGAAGACACTGCTATGTACTATTGC |
| | | GCCAAGGCCATCGTCCCCACAGGTGCCACAATGGAGAGGGGCCAGGGCACGCAG |
| | | GTGACAGTCAGCAGT |
| DR593- | 1063 | CAGGTGCAGGTCCAGGAATCCGGCGGAGGCTCCGTGCAGGCTGGGGGGTCCCTT |
| hIL27 | | AGGTTGTCTTGCGCTGCCAGCGGATACCCTTACAGCAATGGATATATGGGTTGG |
| Ra_VH | | TTCCGCCAGGCCCCTGGCAAGGAGAGAGAGGGAGTGGCCACCATCTACACGGGT |
| H7 | | GACGGGCGTACCTACTATGCCGATTCCGTTAAGGGCAGGTTCACAATCTCCCGC |
| | | GACAACGCCAAGAACACCGTGGACCTGCAAATGTCTTCCCTTAAACCCGAAGAC |
| | | ACTGCCATGTATTACTGCGCTGCCCGCGCGGCCCCACTCTACAGCTCTGGGAGC |
| | | CCTTTGACCCGTGCTCGCTATAACGTGTGGGGCCAGGGTACTCAGGTGACCGTC |
| | | TCAAGCGGCGGAGGUTCTCAGGTGCAGGTCCAGGAGTCAGGTGGAGGTTTGGTG |
| | | CAGCCAGGAGGCTCACTGAGACTGAGCTGCGCTGCCAGCGGGTTCACTTTCTCC |
| | | TCTTATCCTATGTCCTGGGTTCGCCAGGCACCCGGCAAAGGCTTGGAGTGGATC |
| | | AGCACCATCAGTGCGGGGGCGACACAACTTTGTACGCCGACCTGTGTGAAGGGT |
| | | CGTTTTACCTCCAGTCGCGACAATGCTAAAAACACACTGTATCTGCAACTGAAC |
| | | TCTCTGAAGACCGAGGACACCGCCATCTATTACTGTGCCAAAAGAATTGACTGT |
| | | AACAGCGGATATTGCTATAGACGCAACTATTGGGGTCAGGGCACACAGGTTACC |
| | | GTGTCCTCA |
| DR593- | 1064 | CAAGTCCAGCTCCAGGAATCCGGCGGTGGCAGCGTTCAAGCCGGTGGCTCCCTG |
| hIL27 | | CGCCTGAGTTGTGCAGCTTCAGGTTATCCCTACAGCAACGGATACATGGGATGG |
| Ra_VH | | TTCCGTCAGGCTCCCGGTAAGGAGCGTGAGGGTGTGGCAACTATCTATACGGGA |
| H7 | | GATGGCAGGACCTACTATGCGGACTCTGTGAAGGGCAGGTTCACGATCTCTCGT |
| | | GATAACGCTAAGAACACCGTCGATCTCCAGATGTCTTCCTTGAAGCCTGAGGAT |
| | | ACGGCTATGTATTACTGCGCCGCGAGAGCTGCCCCGCTGTATTCCTCTGGCAGC |
| | | CCTCTGACTCGGGCACGGTACAACGTGTGGGGCCAAGGCACCCAGGTCACTGTG |
| | | TCCTCTGGCGGTAGCGGTGGCTCTGGCGGTTCAGGTCAGGTGCAGCTGCAAGAA |
| | | TCCGGTGGAGGCCTGGTGCAGCCCGGCGGTTCCCTCCGTCTGTCCTGTGCTGCA |
| | | TCTGGATTCACTTTTTCTAGCTACCCAATGTCATGGGTGCGCCAGGCCCCAGGC |
| | | AAAGGGCTGGAGTGGATCTCAACTATCTCCGCAGGTGGCGATACTACCCTCTAC |
| | | GCTGACTCCGTGAAGGGGCGGTTTACCAGTTCCCGTGACAACGCCAAGAACACT |
| | | TTGTATCTTCAGCCGAACTCATTGAAGACTGAGGACACCGCCATCTATTACTGC |
| | | GCTAAGAGAATCGACTGCAACTCAGGGTACTGCTATAGACGCAACTATTGGGGC |
| | | CAGGGGACTCAGGTGACCGTGTCAAGC |
| DR593- | 1065 | CAGGTGCAGTTGCAGGAAAGCGGCGGTGGCTCCGTGCAGGCTGGGGGCTCTCTG |
| hIL27 | | CGTTTGTCATGTGCCGCAAGTGGATACCCCTACAGCAACGGCTACATGGGCTGG |
| Ra_VH | | TTCAGGCAGGCTCCCGGAAAGGAGCGCGAAGGGGTGGCCACCATCTACACTGGG |
| H8 | | GATGGAAGGACTTACTATGCGGATAGCGTAAAGGGGCGTTTTACTATCTCCAGA |
| | | GACAATGCCAAAAACACAGTTGACCTCCAGATGTCTAGCTTGAAGCCTGAAGAC |
| | | ACTGCCATGTACTATTGTGCAGCGCGGGCAGCCCCGCTGTATAGCTCCGGCTCC |
| | | CCTCTGACACGCGCCCGCTACAACGTGTGGGGACAGGGCACCCAGGTTACCGTG |
| | | AGTTCCGGCGGTGGGTCCCAGGTCCAGCTTCAGGAGTCCGGGGGGGGTTCCGTC |
| | | CAAGTTGGTGGCAGCCTGCGCTGTCCTGCGCCTGCCTCCGGGTTTACTTTCAGT |
| | | TCCTACCCAATGTCATGGGTGCGCCAGGCTCCTGGTAAGGGGCTGGAATGGATT |
| | | TCTACTATCAGTGCAGGCGGAGATACGACCCTCTACGCCGATAGCGTGAAGGGG |
| | | CGGTTCACTTCATCTCGTGATAACGCCAAGAACACCCTGTACCTTCAGCTGAAC |
| | | TCCCTGAAGACGGAAGACACCGCCATTTATTACTGCGCGAAGAGGATCGACTGC |
| | | AACTCCGGCTACTGCTATCGTCGCAACTATTGGGGCCAGGGTACGCAGGTGACG |
| | | GTCAGTTCC |
| DR593- | 1066 | CAGGTGCAGGTCCAGGAGTCAGGCGGTGGCTCTGTGCAGGCCGGAGGGTCTCTC |
| hIL27 | | CGGTTGAGTTGTGCAGCTTCAGGCTACCCTTATAGCAACGGCTACATGGGATGG |
| Ra_VH | | TTCAGACAGGCACCCGGTAAGGAACGCGAGGGCGTAGCCACGATCTACACTGGT |
| H8 | | GATGGGAGGACCTATTACGCTGACTCTGTGAAAGGTCGGTTCACAATTAGTCGC |
| | | GATAACGCTAAGAACACCGTGGATCTCCAGATGAGCAGTTTGAAGCCTGAGGAC |
| | | ACCGCAATGTACTATTGTGCTGCACGCGCGGCCCCTCTCTACAGTTCCGGCAGC |
| | | CCACTGACTAGAGCCCGGTACAACGTCTGGGGCCAAGGCACCCAGGTCACCGTC |
| | | AGTAGCGGCGGGAGCGGAGGCTCCGGTGGCTCCGGCCAGGTGCAGCTCCAGGAG |
| | | TCCGGAGGCGGATCTGTCCAGGTTGGAGGGAGCCTGCGCTTGAGTTGCGCTGCC |
| | | AGCGGATTCACCTTTTCTTCATACCCCATGAGCCTGGGGGGCCAAGGGCCCGGA |
| | | AAGGGGCTGGAGTGGATCTCAACGATCAGCGCCGGAGGTGATACAACCCTCTAC |
| | | GCAGACAGCGTCAAAGGCAGGTTTACATCTAGCCGCGACAACGCAAAGAACACC |
| | | CTGTATTTGCAGCTGAACAGCGTGAAAACGGAAGACACCGCCATGTATTACTGC |
| | | GCCAAAAGAATTGATTGCAATTCTGGTTATTGTTACAGACGCAATTACTGGGGA |
| | | CAGGGGACCCAGGTTACAGTCTCCAGT |
| DR593- | 1067 | CAGGTCCAGCTTCAGGAAAGCGGCGGGGGTAGCGTCCAGGCAGGTGGCAGCCTG |
| hIL27 | | CGTCTGAGCTGTGCGGCCAGCGGTTATCCTTACTCCAACGGTTACATGGGGTGG |
| Ra_VH | | TTCCGCCAGGCCCCTGGCAAGGAGCGTGAGGGAGTGGCCACCATCTACACTGGA |
| H9 | | GATGGAAGAACCTACTATGCAGATTCAGTTAAGGGCAGGTTTACAATCAGCCGG |
| | | GATAATGCCAAGAACACAGTCGATCTCCAGATGTCCTCTCTGAAGCCCGAAGAC |
| | | ACTGCCATGTATTACTGTGCTGCACGGGCTGCCCCGCTGTATTCTTCAGGTAGT |
| | | CCCCTCACACGCGCCCGCTACAACGTGTGGGGGCAGGGTACACAGGTAACTGTG |
| | | TCTAGCGGCGGTGGGAGCCAGGTCCAGGTCCAGGAGTCTGGTGGCGGATGTGTG |
| | | CAGAGCGGGGGTTCCCTGCGCCTGTCCTGTGCTGCCTCTGGGTTCACGTATAGG |
| | | ACTTCCAACAGCTGGATGGCCTGGTTCCGTCAAGCGCCTGGAAAGGAGCGTGAG |
| | | GGGGTGGCTGCCATCTATACAGTGGGAGGTTCTATCTTTTACGCAGACAGCGTG |
| | | CGTGGACGTTTCACAATCTCTCAAGACGCTACTAAGAATATGTTTTACCTTCAG |
| | | ATGAACACTCTCAAGCCCGAGGACACCGCAATGTATTACTGCGCTGCGGCATCT |
| | | GGCCGCCTGAGAGGCAAGTGGTTTTGGCCCTACGAGTATAATTACTGGGGCCAG |
| | | GGCACGCAGGTGACGGTCTCTAGT |
| DR593- | 1068 | CAAGTTCAACTTCAGGAGAGTGGGGGTGGCTCTGTGCAGGCCGGTGGCTCTCTG |
| hIL27 | | AGACTCAGTTGTGCTGCCTCCGGTTACCCTTATTCCAACGGCTACATGGGCTGG |
| Ra_VH | | TTCCGTCAGGCCCCAGGCAAGGAGCGCGAAGGCGTCGCCACCATTTACACAGGC |
| H9 | | GACGGACGCACATACTATGCTGATTCTGTGAAGGGCCAGATTACCATCAGCCGG |
| | | GATAACGCCAAAAATACCGTGGACCTTCAAATGAGTTCTCTGAAGCCTGAGGAT |
| | | ACCGCCATGTATTACTGCGCCGCGGGGGCAGCTCCCCTCTACAGCAGTGGTTCC |
| | | CCGCTGACCCGCGCCCGCTATAACGTGTGGGGCCAAGGCACACAGGTGACCGTG |
| | | TCCAGCGGGGGCTCCGGGGGCTCTGGGGGCTCAGGACAGGTCCAGTTGCAGGAA |
| | | TCTGGCGGTGGCAGCGTTCAGAGTGGAGGTTCCCTGCGGCTCTGTTGTGCCGCT |
| | | TCCGGCTTCACCTACTCAACCTCTAATAGCTGGATGGCCTGGTTCCGTCAAGCC |
| | | CCCGGCAAGGAGCGCGAAGGCGTGGCTGCAATCTACACCGTAGGCGGATCTATC |
| | | TTCTACGCCGATTCAGTGCGCGGCAGGTTTACCATCTCACAGGACGCCACTAAG |
| | | AATATGTTTTATTTGCAGATGAATACTGTGAAGCCTGAGGACACCGCTATGTAT |
| | | TACTGCGCTGCGGCCAGTGGCCGCCTGAGGGGCAAGTGGTTCTGGCCATACGAA |
| | | TACAACTACTGGGGACAGGGGACCCAGGTTACAGTTAGTAGC |
| DR593- | 1069 | CAGGTGCAGTTGCAGGAATCTGGGGGTGGCAGTGTGCAGGCTGGAGGCTCCCTG |
| hIL27 | | CGCCTGTCCTGTGCGGCCTCTGGCTATCCATATAGTAATGGTTACATGGGATGG |
| Ra_VH | | TTTAGGCAGGCTCCGGGCAAGGAGCGCGAGGGCGTGGCCACTATCTACACTGGT |
| H10 | | GATGGTCGGACCTACTATGCTGACTCTGTGAAGGGGCGCTTCACAATTTCTCGG |
| | | GATAACGCCAAAAATACCGTGGACTTGCAGATGAGTTCCCTCAAACCGGAAGAC |
| | | ACCGCCATGTATTACTGTGCAGCTAGGGCGGCACCGCTGTATAGTTCTGGCAGC |
| | | CCTCTGACTCGGGCTCGCTATAATGTCTGGGGCCAGGGAACCCAGGTTACAGTC |
| | | TCCAGCGGGGGTGGGTCCCAAGTGCAGTTGCAGGAATCCGGTGGGGGTTCCGTT |
| | | CAGGCCGGAGGCTTCACTGCGGCTGTCTTGCAGAGCGTCCGGCTCCACTACTCT |
| | | AACTATTGCCTGGGCTGGTTCCGTCAGATTACTGGCAAGGAGAGGGAGGGCGTG |
| | | GCCGTCATAAATTGGGGGGAGGTATGGTGTACCTTTGCCGATTCAGTTAAAGGA |
| | | CGTTTTACGGTGAGCCAGGACCAGGCTAAGAACACCCTTTACCTTCAGATGAAC |
| | | AGCCTGAAGCCCGAAGACACAGCCATGTACTATTGTGCTGCGGAGTCCGTGTCT |
| | | AGCTTTTCTTGCGGAGGCTGGGTCACGAGACCGGATAGAGTGCCATACTGGGGC |
| | | CAGGGCACCCAGGTGACCGTCTCCAGC |
| DR593- | 1070 | CAGGTGCAGCTCCAGGAAAGCGGAGGGGGGTCTGTCCAGGCAGGGGGCTCCCTG |
| hIL27 | | AGACTCTCTTGTGCTGCGAGCGGTTATCCTTATAGCAACGGGTATATGGGATGG |
| Ra_VH | | TTTCGCCAGGCTCCGGGGAAGGAGCGCGAGGGTGTGGCTACCATCTATACGGGT |
| H10 | | GATGGTCGGACTTACTATGCAGACAGTGTCAAGGGCCGGTTCACCATCAGCAGA |
| | | GACAACGCAAAAAATACCGTGGACTTGCAGATGTCATCCCTCAAGCCAGAGGAC |
| | | ACAGCAATGTATTACTGCGCCGCGAGAGCTGCGCCCCTGTACTCGTCTGGATCT |
| | | CCCTTGACCAGAGCCCGCTATAATGTCTGGGGACAGGGAACCCAGGTAACAGTG |
| | | TCCAGCGGAGGTTCCGGGGGTTCTGGAGGTTCTGGACAGGTTCAACTCCAGGAG |
| | | TCTGGCGGGGGCTCAGTCCAGGCTGGGGGGTCTTTGCGCCTGTCCTGTCGGGCC |
| | | AGCGGCTCCACCTACTCCAACTACTGCCTCGGTTGGTTCCGCCAGATTACTGGC |
| | | AAAGAGCGTGAGGGCGTGGCTGTTATCAACTGGGGGGCGGAATGCCTGTACTTC |
| | | GCCGATTCTGTTAAGGGACGCTTCACCGTCTCTCAGGACCAGGCCAAGAACACG |
| | | CTGTATTTGCAGATGAACAGTCTGAAGCCAGAGGATACAGCAATGTATTACTGT |
| | | GCTGCGGAGTCTGTTAGCTCTTTTTCCTGTGGTGGCTGGCTGACCAGGCGGGAT |
| | | CGCGTGCCTTACTGGGGGCAGGGAACCCAGGTGACTGTCAGCAGT |
| DR593- | 1071 | CAGGTGCAGCTTCAGGAGTCCGGGGGAGGCAGTGTCCAGGCAGGGGGCTCCCTG |
| hIL27 | | CGCCTCTCTTGTGCGGCCTCTGGATATCCCTACTCTAACGGATACATGGGCTGG |
| Ra_VH | | TTTCGTCAGGCCCCAGGAAAAGAGCGGGAGGGGGTGGCGACAATCTACACTGGC |
| H11 | | GATGGCCGCACCTACTATGCTGATTCCGTTAAGGGGCGCTTCACCATCAGCAGA |
| | | GACAACGCAAAGAACACTGTCGATCTTCAGATGAGTTCCCTCAAACCTGAAGAC |
| | | ACAGCCATGTATTACTGTGCAGCCCGCGCGGCCCCTCTTTACAGCTCTGGGAGC |
| | | CCCCTGACACGCGCTCGCTACAATGTTTGGGGTCAGGGCACACAGGTGACAGTC |
| | | TCCTCTGGCGGAGGCTCCCAAGTCCAGCTTCAGGAATCCGGGCGGGGGAGTGTG |
| | | CAAGCTGGTGGATCTCTGCGCCTGAGCTGTAGGGCCTCCGGCAGCACATACTCT |
| | | AACTACTGCCTGGGCTGGTTTAGGCAGTCCACAGGAAAAGAACGCGAAGGAGTC |
| | | GCGGTAATAAATTGGGTCGGGGGTATGCTCTACTTCGCCGACTCCGTGAAAGGC |
| | | CGCTTCACAGTGTCTCAGGATCACGCCAAAAATACCGTAACCCTTCAGATGAAC |
| | | TCCTTGAAGCCAGAGGACACCGCGATGTATTACTGCGCTGCGGAGTCGGTATCC |
| | | AGCTTCAGCTGTGGTGGCTGGCTGACCCGCCCAGGACGGGTTCCATACTGGGGC |
| | | CAGGGTACACAGGTCACAGTGTCTAGC |
| DR593- | 1072 | CAGGTGCCAGCTCCAGGAGTCCGGGGGGGGTCCGTCCAGGCCGGAGGTTCCCTC |
| hIL27 | | CGTCTGTCTTGCGCCGCGTCCGGTTATCCTTACAGCAACGGCTATATGGGTTGG |
| Ra_VH | | TTCCGTCAGGCCCCCGGAAAGGAAAGAGAAGGCGTCGCCACGATCTACACCGGG |
| H11 | | GATGGCAGGACCTACTATGCTGACTCCGTGAAGGGCCGCTTCACAATTAGTCGC |
| | | GACAATGCCAAGAACACGGTGGACCTCCAGATGTCTTCACTTAAACCCGAGGAC |
| | | ACCGCTATGTATTACTGTGCAGCCAGAGCAGCTCCTCTGTACTCCTCAGGCTCT |
| | | CCCCTCACCCGTGCTCGCTACAACGTGTGGGGACAGGGCACACAGGTCACAGTG |
| | | TCATCCGGGGGTTCCGGTGGCAGTGGAGGCAGCGGACAGGTACAGCTTCAGGAG |
| | | TCCGGCGGGGGTTCTGTGCAGGCTGGAGGCTCTTTGCGCCTTAGCTGTCGCGCG |
| | | AGCGGTTCCACCTACTCCAATTACTGCCTGGGTTGGTTCAGGCAGAGCACCGGC |
| | | AAGGAGCGCGAAGGAGTGGCCGTCATCAACTGGGTCGGTGGGATGCTCTATTTT |
| | | GCTGACTCTGTGAAGGGCAGGTTTACTGTGAGCCAGGACCATGCCAAGAACACT |
| | | GTGACTCTCCAGATGAATAGTGTTAAACCAGAGGACACGGCTATGTATTACTGT |
| | | GCAGCCGAATCAGTCTCCTCTTTCTCTTGTGGTGGGTGGTTGACGAGGCCGGGT |
| | | CGTGTTCCCTATTGGGGCCAGGGGACCCAGGTGACTGTATCCTCT |
| DR593- | 1073 | CAGGTGCAGCTCCAAGAGTCCGGGGCGGGGAGCGTGCAGGGGCGGGGTTCCCTT |
| hIL27 | | CGCCTGTCTTGTGCTGCCTCCGGCTACCCCTACTCTAATGGCCACATGGGCTGG |
| Ra_VH | | TTCCGCCAAGCTCCAGGGAAGGAAAGGGAAGGCGTGGCCACTATTTACACGGGC |
| H12 | | GACGGTCGCACATATTACGCTGACTCTGTTAAAGGCCGCTTCACTATCTCCAGA |
| | | GACAACGCAAAGAACACGGTGGACCTCCAGATGTCCTCTCTGAAGCCCGAAGAC |
| | | ACCGCTATGTATTACTGCGCTGCAAGGGCTGCCCCTCTGTACTCCAGGGGTTCC |
| | | CCTCTGACCAGAGCCCGCTATAACGTCTGGGGCCAAGGAACACAGGTCACTGTC |
| | | AGCTCCGGTGGCGGGTCACAGGTGCAGCTTCAGGAGAGCGGGGGGGGCAGTGTC |
| | | CAAGCTGGTGAGAGCCTCAGACTGAGTTGCAGAGCCAGCGGTTCCACCTACTCC |
| | | AACTACTGCTTGGGTTGGTTCAGGCAGATCACAGGCAAGGAGCGTGAGGGCGTC |
| | | GCTGTCATCAACTGGGTGGCGGGGATGCTCTACTTTGCTGACAGCGTTAAGGGC |
| | | CGGTTCACCGTGAGCCAGGATCAGGCGAAGAACACGGTTTATCTGGAGATGAAC |
| | | TCACTGAAGCCCGAGGATACAGCTATGTATTACTGTGCAACTGAGTCAGTTTCC |
| | | TCTTTCTCCTGCGGCGGGTGGCTGACCAGGCCGGATAGGGTGCCGTATTGGGGC |
| | | CAAGGGACTCAGGTTACCGTCTCCTCT |
| DR593- | 1074 | CAAGTGCAGGTCCAAGAATCAGGAGGCGGTTCCGTGCAGGCCGGGGGCTCCCTG |
| hIL27 | | CGCTTGTCTTGCGCGGCCAGCGGGTATCCCTACTCTAATGGTTACATGGGCTGG |
| Ra_VH | | TTCCGTCAGGCTCCTGGCAAGGAAGGGGAGGGAGTGGCCACTATCTATACTGGC |
| H12 | | GACGGGAGGACCTACTATGCCGACTCCGTGAAGGGCCGGTTCACCATCAGCCGC |
| | | GACAACGCAAAAAATACCGTGGACTTGCAAATGAGTTCCCTGAAGCCCGAGGAG |
| | | ACCGCGATGTACTATTGTGCGGCCCGCGCCGCGCCTCTGTACTCCAGTGGGAGG |
| | | CCTCTCACCCGCGCCAGATACAACGTGTGGGGCCAGGGCACCCAGGTCACGGTG |
| | | TCTAGCGGAGGCAGCGGTGGATCTGGTGGCTCTGGACAAGTGCAACTCCAGGAG |
| | | TCAGGCGGTGGCTCCGTGCAGGCTGGCGAGAGCTTGCGGCTGTCCTGCCGCGCG |
| | | TCTGGCAGCACGTACAGCAATTACTGCGTGGGATGGTTCCGGCAGATCACCGGG |
| | | AAAGAGCGTGAGGGAGTCGCCGTGATTAACTGGGTCGGGGGCATGTTGTATTTT |
| | | GCTGATTCCGTGAAAGGGCGCTTTACCGTCAGCCAGGACCAGGCCAAGAACACG |
| | | GTGTATCTTGAGATGAACTCACTCAAGCCTGAGGACACAGCTATGTATTACTGT |
| | | GCGACCGAGTCCGTGTCATCTTTTTCCTGTGGCGGTTGGCTTACAAGGCCTGAT |
| | | CGCGTTCCTTACTGGGGCCAGGGCACTCAGGTGACCGTGTCTAGC |
| DR593- | 1075 | CAGGTACAGTTGCAGGAAAGTGGCGGAGGCAGCGTGCAGGCCGGGGGCAGTCTC |
| hIL27 | | CGTCTGTCTTGTGCCGCTAGTGGCTATCCCTATTCTAACGGCTATATGGGATGG |
| Ra_VH | | TTCCGCCAGGCACCCGGCAAAGAGAGGGAAGGCGTCGCCACCATCTACACAGGT |
| H13 | | GATGGAAGGACATACTATGCAGACAGCGTGAAGGGCCGGTTCACGATTAGCCGC |
| | | GACAACGCGAAGAACACTGTGGATCTCCAAATGAGTTCCCTCAAGCCTGAGGAC |
| | | ACTGCGATGTATTACTGCGCAGCCAGAGCAGGTCCCCTTTACAGCAGTGGAAGC |
| | | CCGCTGACTGGGGCAAGATACAACGTCTGGGGCCAGGGAACTCAGGTTACGGTA |
| | | TCTTCCGGCGGTGGATCTCAGGTTCAGCTTCAGGAATCAGGCGGTGGATCAGTG |
| | | CAGGCGGGCGGAAGTTTGCGCCTGTCCTGTGTAGCCTCCGGGTACGTCCCCTGT |
| | | GACTATTTCCTGCCCAGCTGGTATAGGCAGGCCCCCGGCAAGGAGCGCGAGTTC |
| | | GTGTCCATCATTGATGGCACCGGGTCCACGTCCTACGCCGCTTCTGTGAAGGGG |
| | | AGATTCACGGCGAGCCAAGATCGCGGCAAAAACATCGCATACTTGCAGATGAAC |
| | | TCACTCAAGCCCGAGGATACCGCCATGTATTACTGTAAGGCCAGGTGCGTTCGC |
| | | GGTCGCACTATCTCTGAATATTGGGGCCAGGGAACCCAGGTGACAGTAAGCAGT |
| DR593- | 1076 | CAAGTGCAGCTCCAGGAGAGTGGTGGGGGCTCCGTACAGGCGGGGGGTTCTCTG |
| hIL27 | | CGCCTTAGTTGCGCGGCCAGCGGCTACCCTTATTCCAACGGTTACATGGGGTGG |
| Ra_VH | | TTCCGCCAGGCACCAGGCAAAGAGAGAGAGGGGGTTGCAACCATCTACACTGGC |
| H13 | | GATGGTAGAACTTATTACGCCGACTCTGTAAAGGGGCGCTTCACAATTTCCCGT |
| | | GATAATGCCAAGAACACCGTTGACCTCCAGATGAGCAGTCTGAAGCCAGAGGAT |
| | | ACAGCCATGTATTACTGCGCCGCTCGGGCAGCCCCACTCTACAGGTCCGGTAGC |
| | | CCCCTGACCCGTGCCCGCTACAACGTGTGGGGACAGGGCACACAGGTCACCGTG |
| | | TCCTCTGGTGGCTCCGGGGGCAGCGGGGGATCAGGCCAGGTGCAGCTCCAGGAA |
| | | AGCGGAGGCGGATCTGTGCAAGCCGGAGGCTCTCTGAGACTCTCCTGTGTTGCC |
| | | AGCGGCTACGTGTCCTGTGACTACTTCTTGCCTTCTTGGTATCGTCAGGCACCC |
| | | GGAAAGGAGCGTGAGTTCGTGTCCATCATTGACGGCACAGGCTCTACCTCCTAC |
| | | GCCGCGAGCGTGAAGGGTCGTTTCACCGCTTCACAGGATCGCGGTAAGAACATC |
| | | GCGTATCTCCAGATGAACTCCCTGAAACCCGAAGACACCGCCATGTATTACTGC |
| | | AAAGCCTCCTGTGTGCGCGGGCGCACGATCAGCGAATACTGGGGGCAGGGTACA |
| | | CAGGTCACCGTGTCCTCC |
| DR593- | 1077 | CAGGTGCAGTTGCAGGAGAGCGGTGGAGGCTCCGTTCAGGCAGGTGGCAGCCTG |
| hIL27 | | CGGCTCTCCTGCGCCGCATCCGGTTACCCATACAGCAATGGCTACATGGGATGG |
| Ra_VH | | TTCCGCCAAGCGCCTGGAAAAGAAAGGGAAGGTGTGGCCACTATCTACACTGGC |
| H14 | | GATGGCCGGACGTACTATGCCGACTCAGTGAAGGGCCGGTTCACAATCAGCCGG |
| | | GATAACGCTAAGAACACAGTGGATCTTCAGATGAGTTCCCTGAAACCCGAGGAC |
| | | ACTGCCATGTATTACTGTGCGGCCAGGGCGGCTCCCCTGTACTCTTCCGGCTCC |
| | | CCCTTGACCCGTGCCCGTTATAATGTGTGGGGTCAAGGCACACAGGTCACTGTG |
| | | TCTTCCGGGGGAGGCTCCCAGGTCCAACTCCAGGAAAGTGGTGGAGGCTCTGTG |
| | | CAGGCAGGCGGTTCCCTCCGCCTGAGCTGTGTGGCCTCCGGCTACGTGTCATGT |
| | | GATTACTTTGTGCCAAGCTGGTATCGTCAGGCTCCCGGTAAGGAGCGCGAGTTC |
| | | GTTAGCATTATCGACGGCACCGGCTCCACCAGCTATGCGGCCAGCGTCAAGGGG |
| | | CGCTTCACCGCCTCTCAGGACAAAGGTAAGAACATCGCCTACCTGCAAATGAAC |
| | | TCCCTGAAGCCCGAGGACACAGCCATGTATTACTGCAAGGCCTCCTGTGTCAGA |
| | | GGTAGGGCGATCTCAGAGTACTGGGGTCAGGGAACCCAGGTGACCGTGTCCTCC |
| DR593- | 1078 | CAAGTGCAGTTGCAGGAGAGCGGCGGAGGCTCTGTGCAGGGGGGAGGCAGTCTG |
| hIL27 | | AGACTGTCCTGTGCCGCTTCCGGCTACCCCTATAGCAACGGTTATATGGGTTGG |
| Ra_VH | | TTCCGCCAGGCCCCAGGAAAAGAGCGCGAGGGGGTAGCGACTATTTACACGGGT |
| H14 | | GATGGTAGAACTTACTATGCAGATTCAGTAAAGGGCCGTTTCACCATTTCCCGT |
| | | GACAACGCTAAGAATACCGTAGATTTGCAGATGTCTTCCCTGAAGCCGGAAGAC |
| | | ACTGCCATGTATTACTGCGCTGCACGCGCGGCTCCCCTTTATTCTTCCGGGTCA |
| | | CCACTCACCCGTGCCCGCTACAACGTGTGGGGCCAGGGCACGCAGGTCACTGTA |
| | | TCCAGCGGAGGCAGCGGCGGAAGCGGGGGCTCTGGCCAAGTCCAGCTCCAGGAG |
| | | TCAGGCGGTGGCTCAGTCCAGGCTGGGGGCTCACTGCGCCTGAGGTGCGTGGCC |
| | | TCCGGCTATGTCAGTTGTGATTACTTCCTTCCGAGCTGGTATCGCCAAGCGCCT |
| | | GGGAAGGAGAGGGAGTTCGTGTCTATTATCGACGGGACCGGCAGCACCAGCTAT |
| | | GCGGCTAGTGTGAAGGGCCGTTTCACCGCTTCCCAGGATAAGGGCAAGAACATC |
| | | GCCTATTTGCAAATGAACAGTCTTAAACCGGAGGATACTGCTATGTACTATTGC |
| | | AAGGCCTCCTGCGTGGGGGGAAGAGCCATCAGTGAGTACTGGGGGCAGGGCACC |
| | | CAGGTCACGGTATCCTCC |
| DR593- | 1079 | CAGGTTCAGCTTCAGGAGAGTGGAGGCGGTAGCGTGCAGGCCGGGGGCAGCTTG |
| hIL27 | | CGCCTGTCTTGCGCTGCCTCCGGTTACCCTTATAGTAACGGATACATGGGTTGG |
| Ra_VH | | TTCAGGCAGGCCCCCGGCAAAGAGAGAGAGGGGGTTGCTACCATTTACACCGGC |
| H15 | | GATGGCCGCACCTACTATGCTGACTCTGTGAAGGGTCGGTTCACAATCTCCCGT |
| | | GATAACGCTAAAAATACCGTCGATCTCCAGATGTCTTCCCTGAAACCCGAGGAT |
| | | ACTGCAATGTATTACTGTGCTGCCCGCGCCGCTCCTCTGTACTCTTCAGGTTCT |
| | | CCTCTGACCCGCGCCAGGTACAACGTCTGGGGTCAGGGCACCCAGGTTACCGTA |
| | | AGCTCCGGTGGAGGCTCTCAAGTGCAGTTGCAGGAATCAGGAGGTGGCTCCGTC |
| | | CAGGCAGGTGGGAGCCTCCGCGTCTCATGCGTCGCAAGCGGCTACGTGAGCTGC |
| | | GACTACTTCTTGCCCTCATGGTATCGCCAGGCTCCTGGGAAGGAGCGTGAGTTC |
| | | GTGAGCATCATTGACGGCACCGGCTCCACCAGCTATGCCGCTTCCGTGAAGGGC |
| | | CGGTTCACTGCGTCCCAGGATAAGGGCAAGAACATCGCGTACCTTCAGATGAAC |
| | | ACCCTGAAGCCGGAGGATACAGCTATGTACTATTGCAAGGCATCTTGCGTGAGG |
| | | GGTAGAGCCATCAGCGAGTATTGGGGCCAAGGAACCCAGGTGACTGTTTCCTCC |
| DR593- | 1080 | CAGGTGCAGTTGCAGGAGTCCGGTGGGGGATCTGTGCAGGGGGGAGGGAGCCTG |
| hIL27 | | CGTCTTAGCTGCGCCGCAAGCGGATACCCCTACTCCAACGGATACATGGGTTGG |
| Ra_VH | | TTTCGCCAAGCCCCTGGGAAGGAAAGGGAGGGCGTGGCCACCATGTATACCGGA |
| H15 | | GATGGCCGCACCTATTACGCTGATTCCGTGAAGGGACGTTTCACAATCAGCAGA |
| | | GATAACGCTAAGAACACTGTGGACCTCCAGATGTCTAGCCTGAAACCCGAAGAC |
| | | ACTGCCATGTATTACTGCGCGGCCAGAGCAGCCCCTCTGTATTCCTCTGGAAGC |
| | | CCGTTGACCAGAGCCCGCTACAATGTGTGGGGACAGGGCACCCAGGTCACTGTG |
| | | TCATCCGGGGGAAGCGGGGGTAGTGGGGGCTCTGGCCAAGTTCAGCTCCAGGAA |
| | | AGCGGCGGAGGCAGCGTGCAGGCTGGGGGATCACTGAGATTGAGCTGTGTTGCT |
| | | TCCGGCTATGTGAGCTGTGATTATTTCCCCCCAGCTGGTACAGACCAGGGTCCC |
| | | GGCAAGGAACGCGAGTTTGTCAGTATCATTGATGGCACCGGCTCTACCAGTTAC |
| | | GCTGCGTCAGTTAAAGGACGCTTCACCGCGTCCCAAGACAAAGGCAAGAACATC |
| | | GCCTATTTGCAGATGAACACCCTGAAGCCAGAAGACACTGCCATGTATTACTGC |
| | | AAGGCTTCTTGCGTGCGCGGTCGCGCCATTTCTGAATACTGGGGCCAGGGCACT |
| | | CAAGTCACCGTGTCTTCC |
| DR593- | 1081 | CAGGTGCAGGTCCAGGAGTCTGGAGGCGGAAGTGTCCAGGCTGGGGGTAGCCTG |
| hIL27 | | CGGCTTTCATGTGCCGCTTCCGGCTACCCGTACTCAAACGGCTACATGGGCTGG |
| Ra_VH | | TTCAGACAAGCGCCTGGAAAAGAGAGAGAAGGCGTGGCCACCATCTATACAGGT |
| H16 | | GATGGCCGTACTTATTACGCTGACTCCGTCAAGGGCCGGTTCACCATCAGCCGG |
| | | GATAACGCTAAGAATACCGTCGATCTTCAAATGAGTTCCTTGAAGCCGGAAGAC |
| | | ACCGCTATGTATTACTGTGCCGGGCGGGCTGCCCCGCTGTATAGTAGCGGCTCT |
| | | CCCCTCACCCGCGCTCGCTACAACGTGTGGGGACAGGGGACTCAAGTAACCGTG |
| | | AGTTCCGGCGGTGGCTCTCAGGTGCAGCTGCAAGAAAGCGGCGGGGGCTCCGTG |
| | | CAGGCAGGCGGAAGCGTGCGCCTGTCTTGCAGAGCCTCCGGCAGTACCTACAGT |
| | | AACTATTGTCTGGGCTGGTTTCGCCAGATCACAGGTAAAGAGAGAGAAGGTGTT |
| | | GCCGTTATCAACTGGGTAGGTGGAATGCTGTACTTTGCCGATTCCGTGAAGGGT |
| | | CGCTTCACCGTGTCCCAAGACCAAGCCAAGAATACTGTATATTTGCAGATGAAC |
| | | TCCCTGAAGCCCGAGGACACAGCTATGTACTATTGTGCCGCTGAGTCTGCTAGT |
| | | AGCTTTTCCTGCGGGGGTTGGCTGACCCGCCCGGACCGCGTCCCCTACTGGGGA |
| | | CAGGGCACCCAGGTAACCGTGAGTTCT |
| DR593- | 1082 | CAGGTGCAGTTGCAGGAGTCTGGGGGTGGAAGCGTGCAAGCCGGGGGATCTCTG |
| hIL27 | | CGGCTGAGCTGCGCTGCCAGTGGATATCCATATTCCAACGGCTATATGGGCTGG |
| Ra_VH | | TTTCGCCAAGCGCCCGGCAAGGAGCGCGAAGGCGTCGCCACAATCTACACAGGC |
| H16 | | GATGGCCGCACCTATTACGCTGATTCCGTTAAAGGACGGTTCACGATCAGCCGC |
| | | GACAACGCCAAGAACACAGTTGACCTCCAGATGTCCAGCCTGAAGCCCGAGGAT |
| | | ACTGCGATGTATTACTGTGCCGCGAGAGCTGCCCCACTCTACAGTTCCGGGAGC |
| | | CCCCTTACTCGCGCCCGTTACAATGTTTGGGGACAGGGCACTCAAGTGACAGTG |
| | | TCCAGCGGAGGTTCCGGTGGCTCCGGCGGGTCTGGACAGGTGCAGTTGCAGGAG |
| | | TCCGGCGGAGGGAGCGTACAGGCCGGGGGCTCTTTGCGTCTGTCTTGCAGAGCT |
| | | TCCGGGTTCTACCTATTCCAATTATTGTCTGGGTTGGTTCCGCCAGATACTGGC |
| | | AAGGAGCGTGAAGGGGTCGCCGTCATCAACTGGGTGGGCGGGATGCTCTATTTC |
| | | GCGGATAGTGTGAAGGGCCGCTTTACAGTCTCTCAGGACCAGGGTAAGAATACT |
| | | GTCTATCTTCAGATGAACTCTTTGAAGCCCGAGGATACGGCCATGTACTATTGC |
| | | GCCGCGGAAAGTGCCTCTAGCTTCAGCTGTGGGGGTTGGCTCACCCGCCCAGAC |
| | | CGCGTCCCCTACTGGGGCCAGGGAACCCAGGTGACAGTGTCCAGT |
| DR593- | 1083 | CAGGTTCAGCTGCAAGAATCCGGGGGCGGGTCCGTCCAAGCGGGAGGCAGTCTC |
| hIL27 | | AGACTTTCCTGCGCAGCTTCTGGATACCCTTACTCTAACGGATATATGGGTTGG |
| Ra_VH | | TTTAGACAGGCACCCGGCAAGGAGAGGGAAGGCGTGGCCACGATCTACACTGGG |
| H17 | | GATGGCAGGACCTATTACGCAGACAGCGTGAAGGGCCGCTTCACCATTTCCAGA |
| | | GATAATGCTAAGAACACCGTTGATCTCCAGATGAGTTCCCTGAAGCCAGAAGAT |
| | | ACCGCCATGTATTACTGCGCTGCCCGCGCCGCGCCTCTTTACAGCTCTGGGAGC |
| | | CCGCTGACTCGTGCTAGGTATAACGTCTGGGGCCAGGGAACCCAGGTGACTGTC |
| | | AGCTCTGGGGGCGGGAGCCAGGTGCAGCTCCAGGAGAGTGGCGGTGGCCTTGTT |
| | | CAGCCCGGTGGCTCCTTGCGGCTGTCATGTGCTGCCTCCGGCTTCACATTTTCC |
| | | CTGAGCGGGATGTCCTGGGTGGGGCAGGCCCCCGGCAAGGGCCTTGAGTGGGTC |
| | | TCCGCTATCTCCAGCGGCGGTGCAAGCACTTACTATACCGACAGCGTCAAGGGC |
| | | CGTTTCACTATCTCACGCGATAACGCCAAAAACATGCTGTATCTCCAGCTGAAC |
| | | TCCCTCAAGACCGAGGATACCGCTATGTACTATTGTGCCAAAGGCGGTTCCGGC |
| | | TATGGCGATGCGTCCCGCATGACTTCTCCCGGCTCCCAGGGCACTCAGGTTACC |
| | | GTGTCTAGC |
| DR593- | 1084 | CAAGTTCAGCTTCAGGAGAGTGGAGGCGGATCTGTCCAGGCAGGTGGCTCACTT |
| hIL27 | | AGGCTGTCTTGTGCTGCCAGTGGCTACCCCTACTCCAACGGATATATGGGTTGG |
| Ra_VH | | TTTCGCCAGGCACCAGGGAAGGAGCGCGAGGGCGTCGCTACCATCTACACTGGC |
| H17 | | GACGGACGCACCTACTATGCGGACAGCGTGAAGGGCCGGTTCACCATCAGCCGC |
| | | GACAACGCCAAAAACACCGTCGATTTGCAGATGTCTTCCCTGAAGCCTGAAGAC |
| | | ACAGCCATGTATTACTGTGCCGCGCGTGCTGCCCCACTCTACTCCTCTGGGAGC |
| | | CCCCTCACCCGTGCCCGCTACAACGTGTGGGGCCAGGGCACACAGGTGACCGTT |
| | | TCCAGTGGTGGCAGCGGTGGCAGCGGGGGTTCCGGCCAGGTCCAGCTCCAGGAG |
| | | TCAGGGGGGGGCTGGTGCAGCCCGGGCGGTAGCCTGCGCTTGTCCTGCGCTGCC |
| | | AGCGGGTTCACCTTCAGTCTGTCAGGTATGTCCTGGGTAAGGCAGGCTCCCGGT |
| | | AAGGGCCTGGAGTGGGTATCCGCCATCTCTAGCGGTGGAGCCAGCACCTATTAC |
| | | ACTGATTCAGTAAAAGGACGCTTCACCATTAGCAGGGACAACGCCAAAAACATG |
| | | CTGTATCTTCAACTGAACTCCCTCAAGACTGAGGACACAGCCATGTACTATTGC |
| | | GCCAAGGGAGGGTCTGGCTACGGCGATGCCTCCAGGATGACCTCCCCCGGTAGC |
| | | CAGGGCACTCAGGTGACAGTGTCAAGC |
| DR593- | 1085 | CAGGTGCAGCTCCAGGAGAGCGGTGGGGGAAGCGTGCAGGCTGGCGGAAGCCTC |
| hIL27 | | CGCCTGTCCTGTGCGGCATCTGGCTACCCATATTCTAACGGTTATATGGGCTGG |
| Ra_VH | | TTTCGGCAAGCGCCTGGGAAGGAGCGCGAGGGGGTCGCCACCATTTATACTGGC |
| H18 | | GATGGCCGCACCTATTACGCCGATTCTGTGAAGGGCCGGTTCACGATTTCTAGG |
| | | GATAACGCTAAAAACACCGTGGATCTGCAAATGTCCAGCCTGAAGCCAGAAGAC |
| | | ACAGCCATGTACTATTGCGCCGCTCGGGCCGCTCCTTTGTACTCATCCGGCTCA |
| | | CCTCTCACTAGAGCCCGTTACAACGTCTGGGGACAGGGTACTCAGGTTACCGTA |
| | | AGCTCTGGGGGTGGCTCACAAGTGCAGTTGCAGGAGAGCGGTGGAGGTTCCGTT |
| | | CAGGGTGGAGGCTCCGTGCGGCTTTCCTGCGTGGCTTCCGGCTACGTGTCATGC |
| | | GACTACTTCCTGCCTAGCTGGTATCGCCAGGCTCCTGGGAAGGAGCGTGAATTT |
| | | GTTAGCATCATTGATGGCACAGGCAGCACTAGCTACGCGGCTTCTGTTAAGGGA |
| | | CGTTTTACAGCGTCTCAGGACAAAGGGAAGAACATCGCCTACCTCCAGATGAAC |
| | | TCCCTGAAACCGGAGGATACCGCCATGTACTATTGCAAGGCCTCCTGGGTGCGC |
| | | GGTCGCGGTATTAGCGAATACTGGGGACAGGGCACCCAGGTCACTGTGTCTTCA |
| DR593- | 1086 | CAGGTCCAGCTCCAGGAATCCGGTGGAGGCTCCGTGCAGGCCGGAGGGTCTCTC |
| hIL27 | | AGACTCAGCTGCGCTGCCAGTGGTTATCCGTACTCTAATGGGTACATGGGTTGG |
| Ra_VH | | TTCCGCCAGGCCCCCGGCAAGGAGAGGGAAGGCGTGGCCACAATCTATACCGGC |
| H18 | | GACGGGCGTACATATTACGCGGATAGTGTGAAGGGACGGTTCACCATCAGCCGT |
| | | GACAACGCCAAGAACACCGTGGACCTTCAGATGTCTTCCTTGAAGCCAGAAGAC |
| | | ACCGCGATGTATTACTGCGCGGCTCGCGCAGCTCCCCTCTATTCCTCTGGCTCC |
| | | CCACTGACCCGCGCCAGATACAATGTTTGGGGCCAGGGCACTCAGGTCACTGTG |
| | | TCCAGTGGCGGTAGCGGCGGTAGTGGTGGCTCCGGTCAGGTGCAGCTCCAGGAG |
| | | TCAGGGGGTGGCTCCGTACAGGCTGGCGGCTCACTCCGTCTGAGTTGTGTAGCC |
| | | AGCGGTTACGTCTCCTGCGATTATTTCCTTCCTTCCTGGTATCGCCAGGGTCCC |
| | | GGCAAAGAGCGGGAGTTCGCTTCCATTATCGACGGAACTGGAACAACATCCTAT |
| | | GCCGCTTCTGTCAAGGGGCGCTTCACCGCCTCACAGGATAAGGGCAAGAATATC |
| | | GCTTACCTCCAGATGAACAGCGTGAAACCAGAAGACACCGCGATGTATTACTGT |
| | | AAGGCGAGCTGTGTTCGCGGGGGGGGGATCTCAGAGTATTGGGGCCAGGGCACA |
| | | CAGGTGACCGTATCCTCC |
| DR593- | 1087 | CAAGTGCAGCTCCAGGAGTCAGGCGGTGGCAGCGTACAGGCCGGAGGCTCCCTG |
| hIL27 | | AGACTGAGCTGTGCAGCCTCTGGCTACCCATACAGCAATGGCTATATGGGATGG |
| Ra_VH | | TTCCGCCAGGCTCCGGGTAAAGAGCGCGAGGGCGTGGCCACTATTTATACCGGG |
| H19 | | GACGGAAGAACCTATTACGCGGATTCCGTGAAAGGCCGCTTCACAATTTCCCGT |
| | | GACAACGCGAAGAACACCGTCGATCTCCAGATGTCTTCCCTGAAGCCAGAGGAC |
| | | ACCGCTATGTATTACTGCGCCGCTAGGGCAGCTCCGCTTTACAGTAGCGGATCT |
| | | CCCCTCACCCGCGCCCGCTACAACGTGTGGGGACAGGGAACACAAGTGACAGTT |
| | | TCCTCAGGCGGTGGCTCTCAGGTGCAGGTCCAGGAATCTGGGGGAGGGTCCGTA |
| | | CAGGCCGGAGGCTCCCTGAGATTGAGCTGCCGCGCAAGTGGGTCCACCTACAGC |
| | | AACTATTGTCTGGGGTGGTTTCGCCAGATTACTGGTAAGGAGAGGGAGGGCGTG |
| | | GCCGTCATCAACTGGGGGCGGGGAATGCTCTACTTCGCCGACTCCGTGAAGGGT |
| | | CGGTTCACCGTTTCACAGGACCAAGCAAAGAACACCGTGTACCTCCAGATGAAC |
| | | AGTCTGAAGCCCGAGGATACCGCCATGTATTACTGTGGGGCTGAATCCGTCAGC |
| | | TCCTTCTCCTGCGGGGCGGGGCTGACACGGCCAGATCGGGTCCCTTACTGGGGA |
| | | CAGGGCACACAGGTGACTGTGTCCTCC |
| DR593- | 1088 | CAGGTGCAGTTGCAGGAGAGTGGAGGTGGCAGCGTGCAGGCCGGAGGCAGCCTT |
| hIL27 | | CGCCTCTCTTGCGCTGCGAGTGGCTACCCCTACAGCAATGGGTACATGGGCTGG |
| Ra_VH | | TTCCGTCAGGCACCCGGTAAAGAAAGAGAGGGCGTGGCTACTATCTACACTGGG |
| H19 | | GATGGCAGGACCTATTACGCCGATAGCGTCAAGGGCCGGTTCACGATTAGCCGT |
| | | GACAACGCCAAAAATACTGTGGACCTCCAGATGTCTTCACTGAAACCTGAGGAC |
| | | ACAGCAATGTATTACTGCGCTGCCCGTGCCGCTCCTCTGTACAGCTCCGGCTCC |
| | | CCATTGACCCGCGCCCGTTACAATGTTTGGGGGCAGGGCACCCAAGTCACCGTG |
| | | TCATCCGGGGGTTCCGGCGGAAGCGGTGGCAGCGGCCAGGTGCAGCTTCAGGAG |
| | | TCTGGTGGCGGTAGCGTTCAGGCAGGTGGCAGTCTGCGTCTGAGGTGCCGGGGG |
| | | AGCGGCAGCACATACTCCAACTATTGCCTGGGCTGGTTTCGGCAGATCACTGGT |
| | | AAGGAGCGCGAGGGCGTGGCTGTCATAAATTGGGTCGGAGGTATGCTGTACTTC |
| | | GCCGACTCAGTTAAAGGACGCTTCACCGTGTCCCAGGACCAGGCTAAGAATACC |
| | | GTGTATCTCCAGATGAACAGTCTGAAACCAGAGGACACAGCAATGTATTACTGT |
| | | GCTGCCGAAAGCGTCTCATCTTTCTCCTGTGGGGGCTGGCTCACACGCCCGGAC |
| | | CGTGTTCCGTATTGGGGTCAGGGAACCCAGGTGACCGTAAGCTCG |
| DR593- | 1089 | CAAGTGCAGGTCCAGGAGTCCGGGGCGGGGTCTGTGCAAGCTGGGGGTTCTCTG |
| hIL27 | | AGGCTGTCCTGCGCTGCATCCGGCTACCCTTACTCCAATGGCTACATGGGTTGG |
| Ra_VH | | TTCAGACAAGCTCCCGGAAAGGAGAGGGAGGGTGTGGCTACCATCTACACCGGC |
| H20 | | GACGGACGCACTTATTACGCAGACTCCGTGAAGGGCCGGTTCACCATTTCTCGG |
| | | GATAACGCTAAGAACACCGTGGACCTTCAGATGTCTTCCCTTAAACCTGAGGAC |
| | | ACTGCCATGTATTACTGTGCCGCTCGGGCCGCGCCACTGTACTCTTCCGGGTCC |
| | | CCACTTACACGTGCTCGTTACAATGTGTGGGGTCAAGGAACCCAGGTGACTGTG |
| | | TCCAGCGGAGGCGGTTCACAAGTGCAGCTTCAGGAAAGCGGAGGGGGGTTGGTG |
| | | CAGCCTGGGGGCTCACTCCGTCTGAGCTGTGCCGCTTCTGGTTTCACCTTCTCT |
| | | TCATATCCTATGAGTTGGGTGAGACAAGCGCCGGGCAAAGGACTGGAGTGGGTC |
| | | TCCACTATTTCTTCCGGCGGTGATACTACCTTGTATGCTGACTCCGTGAAGGGT |
| | | CGTTTCACCTCCAGTCGCGACAATGCGAAGAATACCCTGTACCTCCAGCTCAAC |
| | | TCCCTGAAGACCGAGGACACAGCGATGTATTACTGCGCTAAGCGTATCGACTGC |
| | | AATTCCGGGTACTGCTACAAGAGAAGCTACTGGGGGCAAGGGACTCAGGTGACA |
| | | GTGTCTAGC |
| DR593- | 1090 | CAGGTCCAACTTCAGGAGTCTGGTGGAGGCTCCGTTCAGGCGGGGGGGTCCCTG |
| hIL27 | | CGCTTGTCTTGTGCAGCCAGCGGTTACCCATACAGCAACGGATATATGGGCTGG |
| Ra_VH | | TTCCGGCAGGCCCCCGGGAAAGAACGTGAGGGCGTCGCGACTATCTATACTGGA |
| H20 | | GATGGACGCACTTATTACGCCGATAGCGTCAAGGGACGGTTTACCATCAGCAGG |
| | | GACAACGCCAAAAACACCGTGGACCTTCAGATGTCTTCCCTGAAACCCGAGGAC |
| | | ACGGCTATGTACTATTGCGCGGCCCGCGCTGCCCCTCTCTATAGTTCTGGGAGT |
| | | CCCTTGACAAGGGCCAGATACAACGTGTGGGGGCAGGGGACTCAGGTTACTGTC |
| | | TCCTCTGGTGGCTCCGGTGGCAGCGGAGGCTCTGGCCAGGTGCAGTTGCAGGAG |
| | | AGTGGGGGTGGCCTGGTGCAGCCTGGTGGCAGCCTGCGCCTGTCTTGCGCAGCC |
| | | TCTGGGTTTACCTTTAGCTCTTACCCCATGTCCTGGGTCCGCCAGGCTCCCGGC |
| | | AAAGGACTGGAGTGGGTGTCCACCATTTCTAGCGGAGGCGATACTACCCTGTAC |
| | | GCCGACTCTGTGAAGGGGCGCTTCACTAGCTCACGGGACAATGCTAAGAACACA |
| | | CTGTACCTTCAGCTGAACTCACTGAAAACCGAAGACACTGCTATGTATTACTGC |
| | | GCTAAACGCATTGATTGCAACAGCGGCTATTGTTATAAAAGATCCTACTGGGGC |
| | | CAGGGCACCCAGGTCACGGTCTCCAGC |
| DR593- | 1091 | CAAGTTCAGGTCCAGGAGAGTGGAGGGGGCAGTGTTCAGGCCGGAGGTAGCCTG |
| hIL27 | | CGCCTCAGCTGTGCAGCCAGTGGCTACCCCTACTCTAATGGCTACATGGGCTGG |
| Ra_VH | | TTCAGGCAGGCCCCCGGAAAAGAGCGTGAGGGTGTGGCTACAATTTATACCGGA |
| H21 | | GACGGCAGAACCTACTATGCCGACTCCGTGAAGGGCCGGTTCACAATCTGTCGG |
| | | GACAACGCTAAGAACACAGTGGATCTTCAGATGAGCAGTCTCAAGCCAGAGGAT |
| | | ACCGCCATGTATTACTGTGCCGCGCGGGCTGCCCCTTTGTACTCATCTGGGAGC |
| | | CCTCTGACCCGCGCCCGCTATAATGTGTGGGGCCAGGGGACCCAAGTGACGGTA |
| | | TCCTCTGGGGGGGGTAGCCAGGTGCAGCTCCAGGAAAGCGGAGGGGGACTGGTG |
| | | CAGCCCGGCGGGAGCGTGCGGCTTAGCTGCGCAGCCTCCGGCTTCACCTTGTCC |
| | | CTCAGTTCCATGAGTTGGGTTCGCCAGGCTCCAGGGAAGGGACTGGAATGGGTG |
| | | TCAGCCATCTCCTCTGGCGGGGCGTCCACTTATTACACAGACTCCGTGAAGGGT |
| | | AGGTTCACCATTTCTAGGGATAATGCCAAGAACATGCTGTACCTCCAGCTGAAC |
| | | AGTCTGAAAACAGAAGACACAGCTATGTACTATTGCGCCAAAGGTGGGTCCGGT |
| | | TATGGCGATGCGTCCAGGATGACTTCCCCTGGGTCCCAGGGCACACAGGTCACC |
| | | GTGAGTAGC |
| DR593- | 1092 | CAGGTGCAACTCCAGGAGAGCGGGGGAGGCTCTGTCCAAGCAGGGGGGTCTCTC |
| hIL27 | | CGTCTGTCTTGCGCCGCAAGCGGATACCCCTACAGCAACGGTTATATGGGTTGG |
| Ra_VH | | TTCCGCCAGGCCCCCGGAAAGGAGCGTGAGGGTGTGGCCACCATCTACACTGGC |
| H21 | | GATGGAAGAACCTATTACGCAGACAGCGTGAAGGGCCGGTTTACCATCAGCCGC |
| | | GACAACGCCAAGAACACCGTTGACTTGCAGATGTCCAGCCTGAAACCTGAGGAC |
| | | ACAGCTATGTACTATTGTGCTGCCAGAGCGGCTCCTCTCTACAGTTCTGGATCT |
| | | CCCCTTACACGGGCTAGGTACAATGTTTGGGGGCAGGGCACCCAGGTCACCGTG |
| | | TCCTCTGGGGGATCAGGAGGCAGCGGGGGCTCTGGTCAAGTGCAGCTCCAGGAG |
| | | TCCGGCGGGGGACTGGTTCAGCCTGGAGGTTCCCTGCGCCTTTCTTGTGCTGCC |
| | | AGCGGCTTCACGTTCTCCCTGAGTTCCATGAGGTGGGTCCGGCAGGCCCCCGGC |
| | | AAAGGCCTGGAGTGGGTTTCCGCTATCTCCAGGGGGGGGGCCTCCACCTACTAT |
| | | ACCGACTCCGTGAAGGGACGGTTCACAATCTCCAGGGACAATGCTAAGAATATG |
| | | TTGTACCTTCAGTTGAACTCCCTGAAAACCGAAGACACGGCCATGTATTACTGC |
| | | GCCAAGGGAGGCTCCGGTTACGGCGATGCCAGCAGAATGACCTCCCCTGGTAGC |
| | | CAGGGGACACAGGTGACTGTATCCAGC |
| DR593- | 1093 | CAGGTGCAGTTGCAGGAGTCCGGCGGAGGCTCCGTTCAGGCCGGTGGCAGCCTC |
| hIL27 | | AGACTGTCATGTGCAGCCTCCGGTTATCCCTATTCCAATGGTTACATGGGATGG |
| Ra_VH | | TTCCGTCAGGCCCCTGGGAAAGAGAGGGGGGGGTCGCCACCGATGTACACCGGC |
| H22 | | GATGGACGCACATATTACGCCGACAGTGTTAAGGGCCGTTTCACCATCCCTCGC |
| | | GACAACGCCAAAAACACAGTTGATTTGCAGATGAGTTCCCTCAAGCCCGAAGAC |
| | | ACCGCTATGTACTATTGTGCAGCCCGCGCCGCTCCTCTCTACAGCTCTGGCTCC |
| | | CCACTGACGAGGGCGCGGTACAACGTGTGGGGCCAGGGCACCCAGGTTACCGTT |
| | | TCTAGTGGCGGTGGCTCCCAGGTCCAGCTCCAGGAGAGTGGCGGAGGCAGCGTG |
| | | CAGGCAGGCGGTAGCCTTCGCCTGAGCTGCCGGGCCTCCGGGAGCACGTACTCT |
| | | AACTACTGTGTTGGATGGTTCCGGCAGACAACTGGGAAGGAGCGCGAGGGAGTC |
| | | GCCGTTATCAACTGGGTAGGGGGAATGCTGTACTTTGCCGATTCCGTCAAGGGA |
| | | CGCTTTACCGTCTCTCAGGATCAGGCCAAGAACACCGTCTACCTCCAGATGAAC |
| | | AGTCTGAAGCCCGAGGACACCGCTATGTACTATTGCGCTGCGGAGAGCGTTTCC |
| | | TCTTTCTCCTGCGGCGGTTGGCTTACCCGCCCCGACCGGGTGCCTTACTGGGGT |
| | | CAGGGCACACAGGTGACCGTCTCCTCC |
| DR593- | 1094 | CAGGTTCAGCTTCAGGAAAGTGGAGGGGGCTCCGTTCAAGCCGGGGGTTCCCTT |
| hIL27 | | AGGCTGTCCTGCGCCGCTTCTGGTTATCCGTATTCAAATGGGTACATGGGATGG |
| Ra_VH | | TTCCGCCAGGCTCCCGGCAAGGAGCGCGAGGGTGTCGCAACAATCTATACGGGC |
| H22 | | GACGGTCGGACCTACTATGCAGACTCTGTCAAGGGCCGGTTCACAATCCCCCGC |
| | | GATAACGCCAAGAACACAGTGGACCTCCAGATGTCATCCCTGAAGCCAGAGGAT |
| | | ACAGCAATGTATTACTGTGCTGCACGTGCCGCTCCCCTGTACTCTAGTGGGAGC |
| | | CCCCTCACCAGAGCGCGGTATAACGTGTGGGGTCAGGGGACTCAGGTAACCGTG |
| | | TCCAGCGGGGGTTCCGGCGGTAGCGGAGGCTCGGGCCAGGTACAGCTCCAGGAG |
| | | AGCGGAGGGGGCTCCGTCCAGGCTGGGGGGTCCCTGCGCCTGTCCTGCCGGGCC |
| | | TCCGGTAGCACATACTCTAACTACTGCCTGGGCTGGTTCAGGCAGACCACTGGA |
| | | AAGGAACGCGAGGGCGTCGCTGTAATCAACCGGGTCGGAGGTATGCTTTACTTC |
| | | GCAGACTCCGTGAAGGGTCGCTTCACAGTGAGCCAGGACCAAGCTAAGAACACT |
| | | GTGTACCTCCAGATGAACTCTCTCAAGCCGGAGGATACCGCCATGTATTACTGT |
| | | GCAGCCGAGAGTGTGTCTAGCTTCTCCTGCGGAGGCTGGCTGACGAGACCCGAT |
| | | AGGGTCCCGTACTGGGGGCAGGGCACTCAAGTCACTGTGAGCAGG |
| DR593- | 1095 | CAGGTACAACTTCAGGAAAGCGGGGGAGGCTCCGTACAGGGGGGAGGCAGCTTG |
| hIL27 | | CGCCTGAGCTGTGCCGCAAGTGGTTACCCGTACAGCAACGGCTACATGGGCTGG |
| Ra_VH | | TTTCGTCAGGCTCCTGGGAAAGAGAGAGAGGGCGTGGCTACGATCTACACGGGT |
| H23 | | GACGGACGCACCTATTACGCCGACTCCGTGAAAGGAAGATTCACCATCAGCCGC |
| | | GATAACGCTAAGAACACAGTTGACCTGCAAATGTCTAGCCTGAAACCGGAGGAC |
| | | ACTGCTATGTACTATTGTGCCGCACGCGCTGCCCCTCTCTATTCTTCCGGCAGC |
| | | CCCCTCACCAGGGCTCGCTACAACGTCTGGGGCCAAGGGACCCAGGTGACTGTG |
| | | TCCAGCGGTGGAGGGAGCCAGGTGCAGCTCCAGGAGAGCGGCGGGGGCTCTGTC |
| | | CAGGCCGGTGGCAGTGTGCGCCTGTCCTGCCGGGCCTCCCGTTCCCCTTACGGG |
| | | AATTATTGCCTGGGCTGGTTCAGACAGTCTACGGGTAAGGAGCGGGAAGGCGTG |
| | | GCAGTTATCAACTGGGTCGGCGGAATGCTGTACTTTGCCGACTCCGTCAAAGGC |
| | | CGCTTCACTGTGTCTCAGGACCATGCAAAGAACACCGTAACCTTGCAGATGAAC |
| | | TCTCTGAAACCCGAAGACACGGCGATGTATTACTGCGCTGCCGAGTCCGTTTCT |
| | | AGCTTCAGCTGTGGGGGTTGGTTGACAAGGCCAGATCGCGTCCCGTACTGGGGC |
| | | CAAGGAACCCAGGTAACCGTAAGCAGT |
| DR593- | 1096 | CAAGTCCAGCTGCAAGAGAGCGGAGGGGGGAGCGTTCAGGCTGGTGGCAGCCTG |
| hIL27 | | CGGCTGAGTTGTGCTGCCAGCGGATACCCCTATTCCAATGGTTATATGGGTTGG |
| Ra_VH | | TTTAGACAGGCACCTGGAAAGGAACGCGAGGGCGTAGCTACTATTTACACTGGA |
| H23 | | GACGGCAGGACTTATTACGCGGATAGCGYGAAGGGCCGTTTCACAATTTCACGC |
| | | GACAATGCCAAAAACACCGTGGATCTGCAAATGTCCAGCCTGAAGCCCGAGGAC |
| | | ACCGCCATGTACTATTGCGCCGCACGCGCGGCTCCCCTGTACTCCTCAGGTAGC |
| | | CCTTTGACTCGCGCACGCTATAATGTGTGGGGCCAGGGCACCCAGGTGACGGTT |
| | | TCTAGCGGAGGGAGCGGGGGTTCTGGTGGCTCAGGTCAGGTGCAGCTTCAGGAA |
| | | TCTGGAGGTGGCTCCGTACAGGCTGGGGGCTCCCTGCGTCTGAGCTGCCGGGCG |
| | | AGCAGGTCCCCTTATGGTAACTACTGTCTCGGTTGGTTCCGCCAATCCACCGGC |
| | | AAGGAGCGCGAGGGAGTGGCCGTTATAAATTGGGTGGGCGGTATGCTGTACTTT |
| | | GCTGACAGTGTTAAGGGACGTTTCACCGTATCTCAGGACCACGCCAAGAACACC |
| | | GTGACATTGCAGATGAACTCTCTCAAGCCAGAGGACACCGCTATGTATTACTGC |
| | | GCCGCTGAGTCAGTGTCCTCTTTCAGCTGCGGCGGGTGGCTCACTCGCCCTGAC |
| | | AGAGTACCCTATTGGGGGCAGGGGACACAGGTCACGGTTTCCAGG |
| DR593- | 1097 | CAAGTACAGTTGCAGGAGTCCGGCGGTGGCAGTGTTCAGGGGGCGGGGTCCCTG |
| hIL27 | | CGCCTCAGCTGCGCAGCCAGCGGATACCCATACTCTAACGGTTACATGGGTTGG |
| Ra_VH | | TTCCGCCAGGCCCCAGGTAAGGAAAGAGAGGGAGTGGCGACTATTTATACCGGG |
| H24 | | GATGGCCGCACCTACTATGCCGATTCTGTAAAAGGTAGGTTCACGATCTCTCGC |
| | | GATAACGCCAAAAACACAGTCGATCTCCAGATGTCTTCCCTGAAGCCCGAGGAC |
| | | ACCGCCATGTATTACTGTGCGGCACGGGCAGCTCCCCTTTACAGCTCCGGTTCC |
| | | CCGCTGACTCGTGCCCGTTACAACGTGTGGGGGCAGGGCACACAGGTAACCGTG |
| | | TCCAGCGGGGGAGGTTCACAGGTGCAGCTCCAGGAGTCTGGTGGGGGACTGGTG |
| | | CAGCCCGGAGGCTCCCTGCGTGTCAGCTGTGCAGCCAGCGGCTTTACATTTAGC |
| | | CACTCCGGTATGAGCTGGGTACGGCAGGCACCTGGAAAGGGTCTGGAGTGGGTG |
| | | TCCACAATCAACTCAGGCGGAGCCTCTACATACTATACTGACTCAGTCAAGGGA |
| | | CGTTTCACCATCTCCCGCGACAACGCTAAAAACATGCTGTACCTTCAGTTGAAC |
| | | AGTCTGAAGACTGAGGATACAGCCATGTATTACTGTGCCAAAGGGGGCTCTGGC |
| | | TACGGGGATGCCTCCAGAATGACTAGCCCTGGCTCCCAGGGCACCCAGGTGACC |
| | | GTGTCTTCC |
| DR593- | 1098 | CAGGTGCAGTTGCAAGAGTCTGGCGGAGGCTCCGTCCAAGCAGGGGGGAGTCTT |
| hIL27 | | CGGCTGTCTTGTGCAGCCTCCGGGTATCCGTACTCTAATGGTTACATGGGCTGG |
| Ra_VH | | TTCAGGCAGGCCCCAGGTAAAGAACGGGAGGGCGTGGCCACCATTTACACCGGC |
| H24 | | GATGGCCGCACATACTATGCCGACTCCGTGAAGGGTAGGTTCACCATTTCACGT |
| | | GACAATGCCAAGAATACCGTGGATCTCCAGATGTCTAGCCTGAAGCCGGAGGAC |
| | | ACAGCCATGTATTACTGTGCTGCCCGCGCTGCCCCTCTGTACTCCAGCGGTTCT |
| | | CCTCTGACTAGAGCCCGGTACAACGTGTGGGGCCAGGGCACCCAAGTCACTGTG |
| | | TCTAGTGGGGGGTCCGGCGGAAGCGGTGGCTCAGGCCAAGTTCAGCTCCAGGAA |
| | | TCAGGTGGGGGCCTGGTCCAGCCCGGTGGATCTCTGCGGCTGTCCTGTGCTGCC |
| | | AGCGGATTCACGTTTAGCCACAGCGGCATGAGCTGGGTACGCCAGGCTCCAGGC |
| | | AAAGGATTGGAATGGGTGTCCACAATTAACTCTGGGGGGGCCAGCACCTATTAC |
| | | ACTGACAGCGTTAAAGGGCGCTTTACAATCTCTCGCGACAACGCCAAGAATATG |
| | | TTGTACCTCCAGCTGAACTCACTCAAGACCGAGGATACTGCCATGTATTACTGT |
| | | GCTAAAGGCGGAAGCGGCTACGGCGACGCTTCCAGGATGACCAGTCCTGGCTCC |
| | | CAGGGCACACAGGTGACCGTGTCCTCC |
| DR594- | 1099 | CAGGTCCAGGTTCAGGAGTCCGGCGGAGGCAGCGTACAGGGGGGCGGGTCCTTG |
| hIL27 | | CGCCTGTCCTGTGTGGCCTCCGCATCTACGTATTGCACCTATGATATGCACTGG |
| Ra_VH | | TATCGTCAGGCCCCTGGCAAAGGTCGGGAGTTCGTGAGCGCCATTGATTCTGAT |
| H1 | | GGCACTACCCGCTACGCCGACAGTGTGAAGGGACGGTTTACCATTTCCCAGGGA |
| | | ACCGCGAAGAATACCGTGTACCTCCAGATGAACAGCCTGCAACCCGAGGACACA |
| | | GCTATGTACTATTGTAAGACCGTGTGTGTCGTGGGTTCCCGCTGGAGCGACTAT |
| | | TGGGGACAGGGCACCCAAGTGACCGTCAGCTCCGGGGGGGGCTCTCAGGTGCAG |
| | | TTGCAGGAGAGCGGGGGTGGATTGGTGCAGCCCGGGGGATCTCTGCGCCTGAGC |
| | | TGCGCCGCTTCCGGGTTCACTTTTTCATCTTACCCAATGTCCTGGGTGCGCCAA |
| | | GCGCCGGGCAAGGGACTGGAGTGGATCTCCACGATCAGCGCTGGCGGTGATACA |
| | | ACCCTTTACGCCGACTCCGTGAAGGGCCGCTTTACCAGCTCAAGAGACAACGCA |
| | | AAAAACACTCTGTACTTGCAGGTGAACTCTCTCAAGACCGAGGATGCGGCCATT |
| | | TATTACTGCGCCAAGCGCATTGATTGTAACTCTGGGTACTGTTATAGACGCAAC |
| | | TATTGGGGTCAGGGCACGCAGGTGACCGTGTCTTCC |
| DR594- | 1100 | CAGGTTCAGCTCCAGGAAAGCGGCGGAGGCAGTGTGCAGGCAGGCGGAAGCCTG |
| hIL27 | | AGACTGTCCTGTGTAGCGTCAGCCAGTACCTACTGCACATACGATATGCACTGG |
| Ra_VH | | TATCGCCAAGCTCCTGGGAAAGGAAGAGAGTTCGTTTCCGCCATCGACTCTGAC |
| H1 | | GGCACTACAAGATATGCCGATAGCGTTAAAGGCCGCTTTACAATTTCCCAGGGC |
| | | ACCGCGAAGAACACCGTCTACGTGCAAATGAATAGTTTGCAGCCAGAAGACACT |
| | | GCTATGTACTATTGCAAGACCGTGTGCGTGGTCGGGTCAAGGTGGAGCGATTAC |
| | | TGGGGCCAAGGTACTCAGGTCACCGTCAGCTCCGGTGGAAGCGGGGGCTCCGGG |
| | | GGCTGTGGACAGGTCCAGCTCCAGGAGTCTGGCGGAGGCCTCGTTCAGCCAGGG |
| | | GGCTCATTGAGGCTTTCCTGGCGCGCCTCCGGCTTCACATTTTCTAGCTACCCG |
| | | ATGAGTTGGGTACGCCAGGCTCCTGGAAAAGGCCTGGAATGGATCAGCACAATC |
| | | AGCGCAGGTGGGGACACAACCCTGTACGCTGATAGCGTGAAGGGTAGGTTTACA |
| | | TCTTCCCGCGACAACGCTAAGAACACCCTTTACTTGCAGCTCAATTCCCTCAAG |
| | | ACCGAGGACGCGGCCATCTATTACTGCGCCAAACGTATTGACTGCAACTCTGGT |
| | | TATTGTTACAGAAGGAATTACTGGGGCCAGGGGACCCAGGTGACCGTATGTTCC |
| DR594- | 1101 | CAGGTGCAGGTCCAAGAATCCGGGGGTGGCAGTGTGCAGGCCGGTGGCTCTCTG |
| hIL27 | | AGGTTGAGCTGCGTCGCCTCAGCCAGCACTTACTGCACCTATGACATGCACTGG |
| Ra_VH | | TACAGACAGGCACCCGGTAAGGGACGTGAGTTCGTGTCTGCTATTGATTCCGAT |
| H2 | | GGCACTACCAGATATGCCGATTCCGTGAAAGGCAGATTCACGATTTCCCAAGGA |
| | | ACCGCTAAGAACACCGTGTATCTCCAAATGAACTCCCTTCAGCCCGAAGACACC |
| | | GCTATGTATTACTGTAAGACCGTGTGCGTAGTGGGCAGTCGTTGGTCAGACTAT |
| | | TGGGGCCAGGGCACCCAGGTCACCGTCAGTTCCGGCGGAGGCTCTCAGGTCCAA |
| | | CTTCAGGAATCTGGGGGAGGTTTGGTGCAGCCGGGTGGCTCCCTGCGGCTGTCC |
| | | TGTGCGGCAAGTGGCTTCACCTTCAGCCTCTCCCGGCATGTCCTGGGGGGCCAA |
| | | GCGCCCGGTAAGGGGGTGGAGTGGGTGTCCGCCATTAGGTCTGGCGGTGGTTCT |
| | | ACCTATTACACCGACTCCGTCAAGGGTCGGTTTACCATCTCTCGCGACAACGCT |
| | | AAGAACATCTTGTACCTTCAGCTCAATTCTCTGAAGACTGAGGACACTGCCATG |
| | | TATTACTGTGCGAAAGGTGGCTCCGGCTACGGAGATGCCTCTCGCATGACCTCC |
| | | CCCGGGTCCCAGGGCACACAGGTTACCGTGTCCTCC |
| DR594- | 1102 | CAGGTCCAGGTCCAGGAGTGTGGCGGAGGGAGGGTTCAGGCCGGGGGCTCTCTG |
| hIL27 | | AGGCTCTCCTGTGTGGCGAGCGCCTCTACTTACTGTACTTACGACATGCACTGG |
| Ra_VH | | TATCGCCAAGCCCCAGGCAAGGGCAGGGAGTTCGTCAGTGCCATCGACTCCGAC |
| H2 | | GGCACAACTCGCTACGCCGACTCCGTCAAGGGACGTTTCACAATCTCTCAGGGC |
| | | ACGGCTAAGAACACTGTGTATCTCCAAATGAACAGTCTCCAGCCTGAAGATACA |
| | | GCCATGTACTATTGCAAAACTGTGTGTGTGGTCGGCTCACGGTGGTCTGACTAC |
| | | TGGGGTCAGGGGACCCAGGTCACAGTAAGCTCAGGAGGTTCCGGCGGTTCTGGC |
| | | GGTTCAGGACAGGTGCAACTTCAGGAAAGCGGTGGCGGACTCGTCCAGCCCGGA |
| | | GGTTCACTGCGTCTCAGCTGTGCTGCCAGCGGTTTTACCTTCAGCCTCAGTGGA |
| | | ATGAGCTGGGTCAGACAGGCCCCTGGCAAAGGGCTGGAGTGGGTGAGTGCCATT |
| | | TCCTCAGGAGGTGCCTTCTACCTATACACCGACTCTGTCAAAGGCAGGTTTACC |
| | | ATCAGCCGCGATAACGCCAAGAACATCCTGTATCTCCAGCTTAATTCCCTCAAG |
| | | ACAGAAGACACAGCTATGTATTACTGTGCGAAAGGGGGCTCCGGTTACGGGGAC |
| | | GCTTCCAGAATGACATCCCCTGGTTCTCAGGGCACACAGGTGACCGTGTCCTCA |
| DR594- | 1103 | CAAGTTCAGCTCCAGGAATCTGGCGGTGGGTCTGTTCAAGCCGGTGGCTCCCTG |
| hIL27 | | CGCCTTTCATGTGTCGCCTCTGCTTCTACATACTGCACCTACGACATGCACTGG |
| Ra_VH | | TATCGTCAGGCTCCCGGCAAGGGCCGCGAATTTGTTAGTGCTATCGACTCCGAC |
| H3 | | GGCACTACACGCTATGCCGACAGTGTTAAAGGCCGCTTCACCATCCCTCAGGGG |
| | | ACGGCGAAAAACACCGTGTACCTCCAGATGAACTCCCTGCAACCCGAGGATACA |
| | | GCCATGTATTACTGCAAGACAGTCTGTGTCGTGGGATCTCGCTGGTCTGACTAC |
| | | TGGGGACAGGGGACCCAGGTTACTGTCTCCTCTGGCGGAGGCAGCCAGGTGCAG |
| | | CTTCAGGAGTCCGGTGGGGGCTCCGTGCAGGCTGGCGGAAGCCTGCGCCTGTCC |
| | | TGCGTTGCATCCGGTTATGTTAGCTGCGATTACTTCCTGCCCTCCTGGTATCGT |
| | | CAAGCTCCCGGCAAGGAGCGCGAGTTTGTTTCCATTATCGACGGCACCGGCAGG |
| | | ACGTCATACGCTGCGAGCGTTAAGGGCCGCTTCACCGGTTCCGAAGACAAGGGC |
| | | AAGAACATCGCCTACTTGCAGATGAACTCCCTGAAGCCTGAGGATACCGCCATG |
| | | TATTACTGCAAGGCCAGCTGCGTCCGGGGCCGGGCCGTGTCCGAGTATTGGGGG |
| | | CAGGGCACCCAGGTGACAGTGAGTAGC |
| DR594- | 1104 | CAAGTCCAACTCCAGGAAAGTGGCGGTGGGTCCGTACAGGCCGGGGGCAGTCTG |
| hIL27 | | CGGCTTTCCTGTGTGGCCTCTGCATCTACTTACTGTACTTACGACATGCACTGG |
| Ra_VH | | TATCGCCAGGCCCCAGGTAAGGGTCGGGAGTTTGTTAGCGCAATCGACTCAGAC |
| H3 | | GGGACTACCCGCTACGCGGATAGCGTCAAGGGCCGGTTCACCATGTCTCAGGGC |
| | | ACCGCCAAGAACACCGTTTATTTGCAGATGAATAGCCTCCAACCTGAAGATACT |
| | | GCAATGTATTACTGCAAGACAGTGTGCGTGGTTGGCAGCCGCTGGTCAGACTAT |
| | | TGGGGTCAGGGCACCCAGGTCACCGTTAGCTCTGGAGGCAGCGGCGGTAGCGGT |
| | | GGCAGCGGACAGGTCCAGCTCCAGGAGTCAGGAGGGGGCTCTGTCCAGGCCGGG |
| | | GGCTCTCTCCGTCTTTCTTGCGTCGCCTCAGGCTACGTCAGCTGCGATTACTTC |
| | | CTCCCTAGCTGGTATCGTCAAGCGCCGGGGAAGGAACGGGAGTTTGTGTCAATC |
| | | ATTGACGGCACCGGCTCTACTTCCTATGCTGCCAGCGTGAAGGGCCGCTTTACA |
| | | GCCTCCGAAGACAAGGGCAAGAACATCGCTTACCTCCAGATGAACTCTCTGAAG |
| | | CCCGAAGACACCGCTATGTACTATTGCAAGGCCAGCTGCGTCAGAGGTAGGGCT |
| | | GTGTCCGAATATTGGGGTCAGGGCACGCAGGTTACCGTGTCTTCT |
| DR594- | 1105 | CAGGTCCAACTCCAGGAGAGCGGAGGGGGTTCCGTGCAAGCAGGTGGCTCCCTG |
| hIL27 | | CGCTTGAGCTGTGTGGCCTCCGCTAGTACTTACTGCACTTACGATATGCACTGG |
| Ra_VH | | TATAGGCAAGCCCCAGGGAAGGGGCGCGAGTTCGTGTCCGCCATTGACAGCGAC |
| H4 | | GGTACAACCCGGTATGCCGACTCTGTGAAAGGCCGTTTCACAATCTCCCAGGGA |
| | | ACAGCCAAGAACACCGTATACCTCCAGATGAACTCCTTGCAGCCTGAGGATACA |
| | | GCCATGTACTATTGTAAAACCGTGTGCGTGGTCGGCTCACGCTGGAGTGATTAC |
| | | TGGGGCCAAGGAACCCAGGTCACTGTCTCCTCAGGGGGAGGTAGCCAGGTCCAG |
| | | CTTCAGGAGTCCGGCGGTGGCCTGGTTCAGCCTGGGGAATCACTGCGTCTCTCC |
| | | TGCACTGCCAGTGGATTCACATTTTCAAACTATGCTATGTCTTGGGTGCGGCAG |
| | | GCCCCTGGGAAGGGGCTCGAATGGGTATCCGGTATTAACGTGGCATACGGCATC |
| | | ACCTCCTACGCTGATTCCGTGAAGGGCAGGTTCACCATCTCTCGCGACAACACC |
| | | AAGAACACACTGTACTTGCAACTTAACAGTCTTAAAACTGAAGACACTGCTATC |
| | | TACTATTGCGTCAAGCACAGCGGTACGACTATCCCGCGTGGATTCATCAGCTAT |
| | | ACAAAGAGGGGCCAAGGCACCCAAGTCACTGTGAGCAGG |
| DR594- | 1106 | CAGGTCCAACTTCAGGAATCTGGCGGAGGCTCTGTGCAGGCTGGAGGGAGTCTT |
| hIL27 | | AGACTGTCCTGTGTCGCAAGCGCAAGCACGTACTGCACGTACGACATGCACTGG |
| Ra_VH | | TATCGCCAGGCTCCGGGTAAGGGACGCGAATTTGTGAGCGCAATCGACTCCGAC |
| H4 | | GGGACCACAAGGTACGCCGATAGCGTTAAGGGACGTTTCACCATCAGTCAGGGT |
| | | ACTGCCAAGAACACGGTCTACCTCCAGATGAATAGCCTCCAGCCGGAAGATACC |
| | | GCTATGTATTACTGCAAGACAGTGTGCGTAGTGGGCAGCCGTTGGTCCGATTAT |
| | | TGGGGCCAGGGTACACAGGTGACAGTCTCCAGTGGAGGGTCCGGCGGATGTGGG |
| | | GGCAGCGGTCAGGTGCAGTTGCAGGAGAGCGGCGGTGGCTTGGTGCAACCCGGT |
| | | GAATCTTTGCGTCTGAGCTGCACAGCGAGCGGATTCACATTTAGCAACTACGCC |
| | | ATGTCCTGGGTGCGCCAGGCTCCCGGTAAGGGTCTGGAGTGGGTGAGTGGTATC |
| | | AATGTGGCCTATGGGATCACCAGTTATGCGGATAGCGTGAAGGGCCGGTTTACC |
| | | ATCAGCCGGGACAACACCAAGAACACCCTGTACCTTCAGCTTAACTCTCTGAAG |
| | | ACCGAAGATACAGCCATCTATTACTGTGTGAAGCACTCAGGGACTACCATCCCT |
| | | CGTGGTTTTATTAGCTACACTAAGCGTGGGCAAGGAACACAAGTGACGGTGTCT |
| | | TCC |
| DR594- | 1107 | CAGGTGCAGCTCCAGGAATCCGGCGGGGGCAGCGTGCAGGCCGGAGGCTCCTTG |
| hIL27 | | CGTCTGTCTTGCGTCGCATCCGCCAGCACGTACTGCACCTACGATATGCACTGG |
| Ra_VH | | TATCGTCAGGCTCCCGGTAAGGGGAGGGAGTTTGTCAGTGCTATCGACAGCGAT |
| H5 | | GGGACCACTCGCTACGCTGACTCCGTGAAGGGTCGTTTTACCATCAGCCAGGGC |
| | | ACAGGTAAGAACACGGTGTACTTGCAGATGAACTCTCTCCAGCCGGAAGACACC |
| | | GCGATGTATTACTGCAAGACTGTTTGCGTCGTGGGCTCTCGTTGGTCTGACTAT |
| | | TGGGGTCAGGGCACCCAAGTCACGGTGAGTAGCGGCGGGGGTAGCCAAGTGCAG |
| | | CTTCAGGAGTCCGGTGGCGGAAGCGTGCAGGCTGGGGGCAGCCTTCGGCTGTCA |
| | | TGTACCGCCTCCGGTTACGTGTCTTGTGACTACTTCTTGCCAAGCTGGTATCGT |
| | | CAGGCCCCTGGTAAAGAGCGGGAGTTCGTCAGCGTGATTGATGGCACTGGGTCC |
| | | ACCAGCTACGCTGCCAGCGTTAAGGGCCGCTTCACCGCCAGCCAGGACAAGGGA |
| | | AAGAATATTGCATATCTCCAGAGAACCTCCCTGAAGCCTGAAGATACCGCTATG |
| | | TATTACTGCAAGGCCTCATGCGTCCGTGGACGCGCTATTTCCGAGTATTGGGGA |
| | | CAGGGTACTCAGGTCACCGTGAGTTCT |
| DR594- | 1108 | CAGGTGCAGGTGCAAGAAAGCGGTGGGGGCTCCGTCCAGGGGGGGGGTAGCCTG |
| hIL27 | | AGGCTGAGTTGCGTCGCCAGCGCTAGTACATACTGTACCTACGACATGCACTGG |
| Ra_VH | | TATCGCCAGGCCCCCGGCAAAGGCAGGGAGTTCGTGTCAGCCATCGACTCTGAC |
| H5 | | GGGACTACCAGATACGCCGACTCCGTGAAAGGGCGGTTTACCATCAGTCAGGGT |
| | | ACAGCTAAGAATACCGTGTACCTCCAGATGAACTCCTTGCAGCCTGAGGACACT |
| | | GCAATGTATTACTGCAAGACCGTGTGTGTTGTCGGCAGCCGTTGGTCCGACTAC |
| | | TGGGGACAGGGAACCCAGGTCACAGTCTCTAGCGGCGGAAGCGGGGGCTCAGGA |
| | | GGCTCTGGGCAAGTGCAGTTGCAGGAGAGTGGCGGAGGCAGTGTGCAGGCCGGG |
| | | GGCTCCCTCCGCCTGAGCTGTACTGCGAGCGGGTACGTCAGCTGTGACTACTTT |
| | | CTGCCATCTTGGTATCGTCAGGCTCCCGGAAAGGAACGCGAGTTTGTGTCTGTG |
| | | ATCGACGGGACCGGCTCTACCTCTTACGCCGCTTCCGTCAAGGGCCGCTTTACC |
| | | GCTTCCCAAGATAAGGGGAAGAATATCGCTTACTTGCAGATGAACTCCCTGAAG |
| | | CCAGAAGACACCGCCATGTACTATTGCAAGGCTTCCTGTGTTCGTGGCCGCGCC |
| | | ATTAGCGAGTACTGGGGCCAGGGCACCCAGGTCACCGTGTCCAGT |
| DR594- | 1109 | CAGGTACAACTCCAGGAGAGTGGTGGCGGGAGCGTGCAAGCAGGGGGCTCCCTG |
| hIL27 | | CGCCTGTCCTGCGTCGCGTCCGCCAGCACATATTGCACCTACGATATGCACTGG |
| Ra_VH | | TATCGCCAGGCCCCTGGGAAGGGTCGCGAGTTCGTGTCCGCCATCGACTCAGAC |
| H6 | | GGCACTACCCGCTACGCGGATTCTGTGAAGGGCAGATTCACAATTTCCCAGGGG |
| | | ACCGCCAAGAACACGGTGTATCTCCAGATGAACTCTTTGCAGCCCGAGGACACT |
| | | GCCATGTATTACTGCAAGACCGTGTGTGTCGTGGGCTCCAGGTGGAGCGACTAT |
| | | TGGGGCCAGGGGACACAGGTTACCGTGTCCAGCGGTGGGGGTTCCCAGGTACAG |
| | | CTCCAGGAGTCTGGCGGTGGCGTGGTACAGCCCGGTGGAAGCCTGCGCCTCAGC |
| | | TGTGCCGCTTCCGGCTTTAGCTTCAGTTCTTATGCCATGAAGTGGGTCCGCCAG |
| | | GCCCCCGGCAAGGGCCTGGAATGGGTCTCCACCATCTCCAGTGGGGGTAGCTCC |
| | | ACCAACTATGCTGATAGCGTGAAGGGACGCTTTACCATCAGTCGTGATAACGCG |
| | | AAGAACACCCTTTACCTTCAGCTGAACTCTCTGAAGATCGAAGACACCGCCATG |
| | | TACTATTGTGCCAAGGCTATCGTGCCTACCGGGGCCACTATGGAAAGAGGACAG |
| | | GGCACCCAGGTGACAGTGTCCTCC |
| DR594- | 1110 | CAGGTGCAGTTGCAGGAGTCTGGTGGCGGGTCAGTGCAGGCTGGAGGCTCCCTC |
| hIL27 | | AGGCTGAGTTGTGTCGCAAGCGCAAGCACATACTGCACCTATGATATGCACTGG |
| Ra_VH | | TATCGCCAAGCTCCTGGCAAAGGTCGGGAGTTTGTGTCTGCTATTGATAGCGAC |
| H6 | | GGCACCACGCGCTACGCGGACTCCGTGAAGGGCAGATTTACAATCTCACAAGGG |
| | | ACGGCGAAAAATACCGTGTACCTCCAGATGAACTCTCTCCAGCCCGAGGATACC |
| | | GCTATGTATTACTGCAAGACCGTCTGCGTCGTGGGTAGCAGGTGGTCCGACTAC |
| | | TGGGGTCAGGGCACTCAGGTGACGGTGTCTTCAGGAGGCTCCGGGGGCTCTGGC |
| | | GGTAGCGGACAGGTGCAGTTGCAGGAGAGCGGCGGGGGTCTGGTGCAGCGTGGG |
| | | GGTTCCCTGAGACTGAGCTGCGCCGCTTCTGGTTTCAGTTTTAGTTCTTACGCT |
| | | ATGAAGTGGGTACGCCAAGCCCCTGGTAAGGGTCTGGAGTGGGTTTCTACGATT |
| | | TCTAGCGGGGGCAGCTCTACAAACTACGCGGATAGCGTGAAAGGACGCTTTACC |
| | | ATCTCCCGCGACAATGCAAAGAACACTTTGTACCTCCAGCTTAATTCACTGAAG |
| | | ATCGAGGACACGGCTATGTACTATTGCGCTAAGGCCATCGTGCCGACCGGAGCC |
| | | ACTATGGAAAGAGGTCAAGGCACCCAGGTGACTGTGTCCTCA |
| DR594- | 1111 | CAGGTCCAGCTCCAGGAGTCCGGCGGAGGCTCTGTGCAGGCAGGGGGCTCTCTG |
| hIL27 | | AGGCTGAGCTGCGTCGCCTCAGCCTCTACCTACTGCACCTATGACATGCACTGG |
| Ra_VH | | TATCGTCAGGCTCCTGGCAAGGGTCGCGAGTTCGTTTCTGCCATTGATTCCGAC |
| H7 | | GGCACCACTAGATACGCTGACTCCGTCAAGGGCCGCTTCACCATCTCCCAGGGG |
| | | ACTGCTAAGAACACCGTGTACCTTCAGATGAACTCTCTTCAGCCTGAAGATACC |
| | | GCGATGTATTACTGCAAGACTGTTTGCGTTGTGGGAAGTCGTTGGTCCGATTAC |
| | | TGGGGCCAGGGTACTCAAGTGACTGTAAGCTCAGGTGGCGGATCTCAGGTCCAG |
| | | CTCCAGGAGTCAGGGGGTGGCCTCGTTCAGCCAGGGGGGTCTCTGAGACTGAGC |
| | | TGCGCTGCCTCTGGATTCACCTTCTCATCTTACCCCATGTCTTGGGTGCGTCAA |
| | | GCGCCTGGGAAAGGTCTGGAGTGGATCAGCACAATTTCAGCTGGTGGAGATACC |
| | | ACTCTCTACGCTGACAGCGTGAAAGGCCGGTTCACCTCCAGCCGCGACAACGCC |
| | | AAGAATACCGTGTACCTCCAGGTTAACTCCCTCAAGACAGAGGACACTGCAATT |
| | | TACTATTGCGCTAAGCGGATTGATTGTAACTCCGGCTACTGCTACAGGCGTAAC |
| | | TATTGGGGCCAGGGGACGCAGGTGACAGTGTCATCC |
| DR594- | 1112 | CAGGTCCAGCTCCAGGAGAGTGGAGGGGGATCTGTGCAGGCCGGGGGCTCCCTG |
| hIL27 | | CGTCTCTCATGCGTCGCCAGCGCATCTACTTACTGCACATACGACATGCACTGG |
| Ra_VH | | TATCGTCAGGCACCGGGCAAGGGCCGTGAGTTCGTCTCTGCGATTGACTCTGAC |
| H7 | | GGAACTACCCGCTACGCCGATTCTGTGAAAGGGCGGTTCACCATCTCTCAGGGC |
| | | ACTGCCAAGAATACCGTCTACCTGCAAATGAACAGCCTCCAGCCAGAGGATACC |
| | | GCCATGTACTATTGCAAGACGGTCTGCGTCGTGGGCTCCAGATGGTCCGATTAC |
| | | TGGGGCCAGGGAACACAAGTGACAGTTAGTTCCGGTGGCTCAGGAGGTTCCGGG |
| | | GGCAGCGGTCAAGTGCAGTTGCAGGAGAGCGGAGGCGGTCTGGTTCAGCGTGGC |
| | | GGTTCTCTTCGCCTGAGCTGCGCTGCCTCAGGCTTTACGTTCTCCTCTTACCCC |
| | | ATGTCCTGGGTGCGGCAAGCTCCGGGGAAGGGCCTGGAGTGGATCTCCACCATT |
| | | AGTGCAGGTGGCGACACCACTCTGTACGCTGACAGTGTAAAGGGCCGCTTCACA |
| | | TCCTCTCGGGACAACGCCAAAAATACACTCTATTTGCAGCTGAATAGCCTGAAA |
| | | ACCGAGGACACGGCCATTTACTATTGTGCAAAGAGAATTGATTGCAACAGCGGA |
| | | TACTGTTATCGCAGGAACTACTGGGGCCAGGGCACCCAGGTGACAGTGTCCTCT |
| DR594- | 1113 | CAGGTGCAGCTCCAGGAGTCCGGGGGGGGCTCAGTGCAGGCCGGAGGCAGCTTG |
| hIL27 | | AGGCTGAGTTGTGTGGCCAGTGCCTCCACATATTGCACCTACGACATGCACTGG |
| Ra_VH | | TATCGGCAAGCCCCTGGTAAGGGACGGGAATTTGTAAGTGCTATTGATTCTGAT |
| H8 | | GGCACTACCCGTTACGCCGACAGCGTGAAAGGCCGCTTCACTATTTCACAGGGG |
| | | ACTGCCAAAAATACCGTGTACCTTCAGATGAACTCTCTTCAGCCCGAGGACACG |
| | | GCGATGTACTATTGCAAGACCGTTTGTGTTGTGGGTAGTCGCTGGTCCGACTAT |
| | | TGGGGCCAGGGTACTCAGGTGACTGTATCCAGCGGCGGAGGCAGCCAGGTGCAG |
| | | TTGCAGGAGTCCGGGGGTGGAAGCGTCCAGGTGGGTGGCAGTCTGCGCCTCTCA |
| | | TGTGCCGCATCCGGCTTCACCTTCTCTAGTTACCCTATGTCCTGGGTCCGTCAG |
| | | GCCCCTGGAAAGGGTCTTGAGTGGATTTCCACAATCTCTGCTGGGGGCGACACA |
| | | ACCCTGTATGCCGACTCCGTGAAAGGCCGCTTCACCTCCAGTCGCGACAATGGG |
| | | AAGAACACCCTTTATTTGCAGGTGAACTCACTGAAGACCGAGGACACCGCAATT |
| | | TACTATTGTGCTAAGCGCATTGATTGCAATAGCGGCTACTGTTACCGGCGCAAT |
| | | TATTGGGGCCAGGGTACTCAGGTGACTGTGTCCTCT |
| DR594- | 1114 | CAGGTGCAACTGCAAGAGAGCGGCGGTGGCAGCGTACAGGCCGGTGGCTCTCTC |
| hIL27 | | CGCCTTAGCTGCGTCGCCAGCGCTTCCACTTACTGTACTTACGACATGCACTGG |
| Ra_VH | | TACAGGCAAGCGCCCGGCAAGGGACGCGAGTTCGTCTCCGCCATCGACAGCGAC |
| H8 | | GGCACCACTCGCTACGCCGATAGCGTTAAGGGTCGCTTCACCATTTCACAGGGG |
| | | ACCGCCAAGAACACCGTCTATGTTCAGATGAACAGCCTCCAGCCAGAGGATACA |
| | | GCCATGTACTATTGCAAGACCGTCTGCGTCGTGGGCTCTCGTTGGAGTGACTAT |
| | | TGGGGCCAGGGCACTCAAGTGACCGTGTCCTCTGGAGGCTCCGGGGGCAGCGGC |
| | | GGGTCCGGTCAGGTGCAACTCCAGGAGTCCGGCGGAGGGTCTGTGCAGGTTGGT |
| | | GGAAGCCTGCGTCTCTCCTGCGCGGCCTCCGGCTTCACTTTCTCCTCTTACCCC |
| | | ATGAGCTGGGTCAGGCAGGCCCCCGGCAAGGGGCTGGAGTGGATCTCTACCATT |
| | | TCTGCCGGAGGGGACACTACCGTGTACGCAGATTCTGTGAAAGGGCGCTTCACC |
| | | AGCTCTAGGGACAACGCCAAGAATACCTTGTATCTCCAGCTCAACTCCCTCAAG |
| | | ACCGAGGACACAGCTATCTATTACTGTGCTAAGAGGATCGACTGTAACTCTGGT |
| | | TATTGTTATCGCAGGAACTATTGGGGACAGGGGACCCAAGTGACCGTCAGCTCT |
| DR594- | 1115 | CAGGTGCAGCTTCAGGAGTCTGGCGGTGGCAGCGTCCAAGCTGGCGGGAGCCTG |
| hIL27 | | CGGCTGTCTTGTGTGGCCTCCGCCTCAACATACTGTACCTATGATATGCACTGG |
| Ra_VH | | TATAGACAAGCGCCCGGCAAGGGCAGGGAGTTCGTCTCCGCTATTGATAGCGAT |
| H9 | | GGAACCACTCGTTACGCGGACTCCGTGAAGGGCCGCTTTACGATCAGCCAGGGC |
| | | ACCGCGAAGAACACCGTGTATCTTCAGATGAACTCTTTGCAGCCCGAGGACACT |
| | | GCCATGTATTACTGCAAGACTGTTTGTGTAGTGGGCAGCAGGTGGAGCGACTAC |
| | | TGGGGCCAGGGAACACAGGTCACAGTAAGCTCTGGTGGAGGGTCTCAGGTGCAG |
| | | CTTCAAGAATCTGGTGGAGGCTCCGTGCAATCCGGTGGCAGCCTGAGGCTGTCC |
| | | TGCGCTGCCTCAGGATTCACTTACAGCACCTCTAACTCCTGGATGGCCTGGTTT |
| | | CGCCAAGCGCCCGGAAAAGAGAGGGAAGGCGTGGCCGCTATTTACACTGTCGGT |
| | | GGCTCCATCTTCTACGCGGATAGTGTAAGGGGCCGCTTCACCATCTCTCAGGAC |
| | | GCCACAAAAAATATGTTCTACCTCCAGATGAATACCCTTAAACCGGAGGACACC |
| | | GCTATGTATTACTGTGCAGCGGCAAGTGGAAGGCTTCGGGGGAAGTGGTTCTGG |
| | | CCTTACGAATACAACTACTGGGGTCAGGGAACTCAGGTCACAGTTTCCTCC |
| DR594- | 1116 | CAGGTCCAGTTGCAGGAATCTGGTGGGGGTTCCGTCCAGGGGGGAGGTTCCCTG |
| hIL27 | | CGCCTCTCCTGCGTGGCCTCTGCCTCTACGTATTGTACCTACGACATGCACTGG |
| Ra_VH | | TATCGCCAGGCCCCAGGTAAGGGCCGCGAGTTCGTTTCCGCCATTGATTCCGAT |
| H9 | | GGGACCACACGCTATGCTGATTCCGTGAAGGGCCGGTTCACCATCAGTCAAGGC |
| | | ACCGCCAAGAACACAGTGTACCTCCAGATGAACTCCCTCCAGCCTGAGGACACA |
| | | GCCATGTACTATTGTAAGACAGTGTGTGTAGTCGGTTCCCGCTGGAGCGACTAT |
| | | TGGGGACAGGGAACCCAGGTGACGGTTTCATCCGGTGGCAGCGGAGGTAGCGGC |
| | | GGTAGCGGACAGGTGCAGTTGCAAGAATCCGGCGGTGGCTCCGTGCAGAGCGGA |
| | | GGCTCTCTGCGGCTCTCTTGTGCGGCCAGTGGGTTCACCTACTCCACCAGCAAC |
| | | TCTTGGATGGCTTGGTTCCGCCAAGCCCCTGGCAAAGAACGCGAAGGCGTCGCT |
| | | GCCATCTACACTGTTGGGGGAAGTATTTTTTACGCTGACTCCGTCCGTGGCCGT |
| | | TTTACCATCTCCCAGGATGCCACAAAAAATATGTTCTACCTCCAGATGAACACC |
| | | CTGAAGCCCGAGGATACCGCAATGTACTATTGTGCTGCCGCATCTGGTCGTCTG |
| | | CGCGGCAAGTGGTTCTGGCCCTACGAATACAACTACTGGGGTCAAGGTACACAG |
| | | GTGACGGTGTCTTCC |
| DR594- | 1117 | CAGGTGCAGCTTCAGGAATCCGGCGGAGGCTCTGTGCAGGGGGGCGGAAGCCTT |
| hIL27 | | CGGCTGTCTTGTGTTGCCTCCGCGAGCACCTACTGCACCTACGATATGCACTGG |
| Ra_VH | | TATCGCCAGGCTCCTGGCAAGGGCCGTGAGTTTGTGAGCGCCATTGATTGCGAC |
| H10 | | GGAACTACGAGATATGCCGACAGTGTGAAAGGTCGTTTCACTATTTCACAGGGC |
| | | ACCGCCAAGAATACCGTCTATCTCCAAATGAACAGTCTGCAGCCGGAGGACACC |
| | | GCCATGTATTACTGTAAGACGGTATGTGTTGTGGGGTCTCGCTGGTCAGACTAG |
| | | TGGGGCCAGGGGACACAGGTGACTGTCAGCTCCGGGGGGGGCTCACAGGTGCAG |
| | | CTGCAAGAGAGCGGCGGTGGCAGTGTTCAGGGGGGCGGTTCTCTCCGCCTGTCT |
| | | TGCAGGGCCTCCGGGAGCACTTACAGCAATTATTGTTTGGGGTGGTTTAGACAA |
| | | ATCACCGGCAAGGAGCGCGAGGGGGTGGCCGTGATAAATTGGGCGGGGGGTATG |
| | | CTCTACTTCGCTGACTCTGTGAAGGGCCGTTTCACAGTGTCCCAGGACCAGGCG |
| | | AAAAACACCCTTTACCTCCAGATGAACTCTCTCAAGCCCGAGGACACCGCCATG |
| | | TATTACTGTGCTGCCGAGTCCGTGTCCTCTTTCAGCTGCGGTGGGTGGCTGACC |
| | | CGCCCTGATAGGGTGCCGTATTGGGGTCAAGGAACTCAGGTCACAGTGTCCAGC |
| DR594- | 1118 | CAAGTGCAGGTCCAGGAGTCCGGCGGAGGTTCTGTCCAGGCCGGAGGCTCACTG |
| hIL27 | | CGCCTGTCCTGTGTCGCTTCTGCGTCTACTTACTGTACTTATGATATGCACTGG |
| Ra_VH | | TATCGCCAGGCTCCAGGTAAGGGCAGGGAGTTCGTGTCTGCTATTGATTCCGAT |
| H10 | | GGCACGACCCGCTATGCCGATTCAGTGAAAGGCCGTTTCACAATCTCTCAGGGA |
| | | ACAGCCAAGAACACGGTGTACTTGCAGATGAACTCTCTGCAACCCGAGGACACC |
| | | GCCATGTATTACTGTAAGACTGTCTGCGTGGTCGGCTCACGCTGGTCCGACTAT |
| | | TGGGGCCAGGGGACCCAAGTTACTGTTTCCAGCGGAGGCAGCGGTGGATCTGGG |
| | | GGCTCCGGCCAGGTGCAGCTCCAGGAATCTGGGGGCGGGTCCGTGCAGGGTGGG |
| | | GGATCTCTGCGCCTGAGTTGTCGCGCAAGTGGGAGTACCTACAGTAATTATTGC |
| | | CTGGGATGGTTCCGCCAGATCACAGGTAAGGAGCGCGAGGGCGTGGCCGTGATT |
| | | AACTGGGTGGGTGGCATGTTGTACTTCGCTGATTCCGTCAAGGGACGTTTCACT |
| | | GTCTCTCAGGACCAGGCTAAGAATACCTTGTATCTTCAGATGAACTCCCTGAAG |
| | | CCTGAAGATACTGCTATGTATTACTGTGCGGCTGAGAGCGTCAGTTCCTTCTCC |
| | | TGTGGGGGCTGGCTGACTCGCCCGGACCGTGTGCCCTACTGGGGCCAGGGCACT |
| | | CAGGTGACCGTGAGCAGC |
| DR594- | 1119 | CAGGTGCAGCTTCAGGAGAGCGGAGGCGGGTCCGTGCAGGCTGGTGGGTCACTG |
| hIL27 | | AGACTGTCATGTGTGGCTTCAGCTTCAACGTACTGTACCTATGACATGCACTGG |
| Ra_VH | | TACAGGCAAGCTCCTGGGAAAGGCCGCGAGTTCGTGTCCGCTATTGACAGCGAC |
| H11 | | GGTACTACCCGTTATGCCGACAGCGTCAAGGGCAGATTTACCATCTCCCAAGGG |
| | | ACGGCCAAAAACACCGTGTATGTCCAGATGAACAGCCTTCAGCCCGAGGACACC |
| | | GCAATGTATTACTGCAAGACCGTGTGTGTTGTGGGGAGCCGTTGGTCCGACTAC |
| | | TGGGGCCAGGGCACCCAAGTGACCGTCTCCTCTGGGGGTGGCTCCCAGGTCCAG |
| | | CTTCAGGAGTCCGGCGGAGGCAGTGTTCAGGCCGGAGGGAGCCTGAGACTGAGC |
| | | TGCCGTGCTTCTGGTAGTACTTACTCTAACTACTGTTTGGGCTGGTTTCGCCAA |
| | | TCCACCGGCAAAGAGCGTGAGGGGGTCGCGGTCATCAACTGGGTCGGAGGTATG |
| | | CTGTACTTTGCCGATAGCGTCAAGGGAAGATTTACCGTCTCCCAAGACCACGCC |
| | | AAGAACACCGTGACCCTGCAAATGAACAGCCTGAAGCCAGAGGATACCGCCATG |
| | | TACTATTGCGCGGCAGAATCCGTCTCCAGCTTTTCTTGCGGAGGCTGGCTCACA |
| | | AGACCAGGGCGCGTGCCTTACTGGGGACAAGGCACGCAGGTGACAGTCAGCTCA |
| DR594- | 1120 | CAGGTGCAGTTGCAGGAGTCTGGGGGCGGAAGTGTGCAGGCTGGAGGCTCCCTG |
| hIL27 | | CGCCTGAGTTGTGTGGCCAGCGCCAGCACTTACTGCACTTATGACATGCACTGG |
| Ra_VH | | TACAGACAAGCCCCTGGCAAGGGTAGGGAGTTCGTGTCTGCTATTGACTCCGAC |
| H11 | | GGGACCACACGTTATGCCGACTCTGTCAAGGGCCGTTTCACTATCTCCCAGGGA |
| | | ACGGCCAAGAACACAGTGTACTTGCAGATGAACTCTTTGCAACCCGAAGACACC |
| | | GCTATGTATTACTGTAAGACTGTGTGCGTCGTGGGTTCCAGGTGGTCAGACTAC |
| | | TGGGGTCAGGGTACTCAGGTCACTGTTAGCTCAGGGGGTTCTGGGGGCTCCGGG |
| | | GGTTCCGGCCAGGTGCAGCTTCAGGAGTCTGGAGGCGGTTCCGTCCAGGGGGGA |
| | | GGCTCTCTTAGACTGTCCTGCCGCGCCAGCGGCAGCACCTACTCCAACTACTGC |
| | | CTCGGATGGTTTCGTCAGTCCACAGGTAAGGAGCGCGAAGGCGTGGCTGTTATC |
| | | AACTGGGGGGCGGAATGCTGTACTTTGCCTGACTCCGTGAAAGGCCGCTTCACT |
| | | GTGTCACAAGACCATGCAAAGAACACCGTCACATTGCAGATGAACTCTCTGAAA |
| | | CCAGAGGATACTGCCATGTATTACTGCGCAGCTGAATCTGTGTCGTCTTTCTCT |
| | | TGCGGTGGCTGGCTGACCCGCCCTGGGAGAGTGCCGTACTGGGGCCAGGGCACC |
| | | CAAGTGACAGTCAGTTCT |
| DR594- | 1121 | CAAGTACAGCTCCAGGAATCTGGAGGGGGTAGCGTGCAGGCCGGGGGCTCCTTG |
| hIL27 | | CGCTTGTCCTGCGTTGCCTCAGCCAGCACCTACTGCACGTACGACATGCACTGG |
| Ra_VH | | TATCGCCAAGCACCAGGGAAGGGCAGAGAGTTCGTGTCTGCCATCGACAGTGAT |
| H12 | | GGCACTACCCGCTACGCCGACTCCGTTAAGGGGCGCTTTACCATTTCCCAGGGA |
| | | ACAGCCAAGAACACCGTGTACTTGCAGATGAACAGCTTGCAGCCTGAAGACACC |
| | | GCCATGTATTACTGCAAGACCGTGTGTGTAGTGGGTTCAAGGTGGTCCGACTAT |
| | | TGGGGACAGGGCACTCAGGTGACTGTGTCTTCCGGGGGAGGCAGCCAGGTACAG |
| | | CTCCAGGAATCCGGTGGGGGCTCCGTTCAGGCTGGCGAGTCCCTGCGCCTGTCC |
| | | TGTAGGGCCTCCGGCTCCACGTATAGTAACTACTGTCTGGGCTGGTTCCGCCAG |
| | | ATCACCGGCAAGGAACGCGAGGGAGTGGCGGTAATAAATTGGGTTGGGGGAATG |
| | | CTGTATTTCGCCGACAGCGTGAAAGGCAGATTCACCGTGTCCCAGGACCAGGGG |
| | | AAGAACACCGTGTATTTGGAAATGAACTCTCTGAAGCCCGAGGATACCGCTATG |
| | | TATTACTGCGCTACCGAAAGCGTAAGCTCCTTCAGCTGTGGGGGTTGGCTGACA |
| | | CGCCGTGATCGGGTTCCCTACTGGGGCCAGGGTACGCAGGTCACCGTTTCCTCC |
| DR594- | 1122 | CAGGTCCAGCTGCAAGAGTCCGGTGGAGGCAGTGTGCAGGCAGGCGGTAGCCTG |
| hIL27 | | CGTTTGTCCTGTGTGGCATCCGCCAGTACCTACTGCACTTACGATATGCACTGG |
| Ra_VH | | TACAGACAAGCCCCCGGCAAAGGCCGGGAGTTCGTTTCAGCTATCGACTCTGAC |
| H12 | | GGGACCACTAGGTATGCGGACTCTGTCAAGGGCCGCTTTACAATTAGCCAGGGC |
| | | ACGGCCAAAAACACCGTATATCTCCAGATGAACAGCCTCCAACCAGAAGACACA |
| | | GCCATGTATTACTGCAAGACTGTATGCGTGGTCGGCTCCCGTTGGTCTGACTAT |
| | | TGGGGCCAGGGAACTCAGGTCACCGTCTCATCCGGTGGGTCTGGTGGCGGGGGG |
| | | GGTTCCGGCCAGGTGCAGCTCCAGGAGTCAGGTGGGGGGAGCGTGCAGGGCGGG |
| | | GAGTCCCTCCGGCTGAGTTGTAGGGCTTCCGGTAGCACTTACTCCAACTACTGC |
| | | CTCGGCTGGTTTCGCCAGATCACGGGCAAGGAGCGCGAGGGCGTGGCCGTAATT |
| | | AACTGGGTCGGAGGGATGCTGTATTTCGCGGACAGCGTTAAGGGACGCTTCACC |
| | | GTGTCCCAGGATCAGGCCAAGAACACCGTCTATCTGGAGATGAACTCCCTGAAG |
| | | CCGGAGGACACAGCCATGTATTACTGCGCAACGGAATCCGTGTCTAGCTTCAGC |
| | | TGCGGTGGGTGGCTCACCCGTCCCGACCGCGTGCCGTATTGGGGGCAGGGGACC |
| | | CAGGTGACTGTGTCCTCC |
| DR594- | 1123 | CAGGTGCAACTCCAGGAGTCAGGCGGTGGCTCCGTCCAGGCGGGAGGTTCCCTG |
| hIL27 | | CGCCTTTCTTGCGTTGCATCTGCGTCCACCTACTGTACCTATGACATGCACTGG |
| Ra_VH | | TATCGTCAGGCACCAGGCAAGGGGAGGGAGTTCGTGTCTGCGATTGATTCTGAT |
| H13 | | GGCACAACTAGGTATGCTGACTCCGTCAAGGGACGGTTCACAATCTCTCAAGGG |
| | | ACCGCCAAGAATACCGTGTATCTCCAGATGAACTCCCTTCAGCCCGAGGACACG |
| | | GCCATGTATTACTGCAAGACCGTTTGCGTCGTGGGTTCCAGGTGGTCTGACTAC |
| | | TGGGGGCAAGGCACGCAGGTGACCGTTAGCTCTGGGGGGGGTAGCCAGGTGCAG |
| | | TTGCAGGAATCTGGTGGAGGTTCTGTGCAAGCAGGAGGGTCTCTGCGCTTGAGT |
| | | TGCGTAGCAAGTGGGTACGTGAGTTGTGATTACTTCCTGCCTTCTTGGTACAGG |
| | | CAGGCTCCGGGCAAGGAACGCGAGTTCGTGTCCATTATCGACGGGACCGGCTCC |
| | | ACCAGCTATGCCGCGAGCGTCAAGGGGCGCTTCACAGCCAGCCAGGATCGCGGG |
| | | AAGAACATCGCCTACTTGCAGATGAACAGCCTGAAACCGGAAGATACAGGTATG |
| | | TATTACTGTAAGGCCTCCTGCGTCCGTGGCCGTACTATTAGTGAATACTGGGGC |
| | | CAAGGCACGCAGGTGACCGTATCTTCC |
| DR594- | 1124 | CAGGTTCAGCTCCAGGAGAGCGGAGGTGGCAGCGTTCAAGCCGGTGGCTCCCTG |
| hIL27 | | CGCCTGTCTTGTGTCGCGTCTGCTTCCACGTACTGCACCTACGACATGCACTGG |
| Ra_VH | | TATCGGCAGGCTCCTGGAAAGGGCCGCGAGTTCGTCTCCGCTATCGACTCTGAC |
| H13 | | GGAACCACTCGCTATGCCGATAGCGTGAAGGGCCGCTTCACCATTAGCCAAGGC |
| | | ACAGCCAAGAACACAGTGTACTTGCAGATGAACAGCCTCCAGCCGGAAGACACT |
| | | GCTATGTACTATTGCAAGACTGTGTGTGTTGTGGGGTCCCGTTGGTCTGATTAT |
| | | TGGGGCCAGGGCACCCAGGTGACCGTCAGTAGCGGGGGCAGTGGCGGGTCAGGA |
| | | GGCTCCGGTCAGGTGCAGTTGCAGGAGAGCGGAGGGGGCAGTGTTCAGGCCGGG |
| | | GGCAGCCTGAGACTGTCTTGTGTCGCCTCCGGGTATGTGTCTTGTGACTACTTT |
| | | CTGCCATCCTGGTATCGGCAGGCCCCTGGGAAAGAAAGAGAGTTCGTGTCCATT |
| | | ATCGACGGCACTGGAAGCACCTCTTATGCTGCCTCTGTGAAGGGTAGGTTTACT |
| | | GCCTGCCAAGACAGGGGGAAGAACATTGGTTATCTCCAGATGAACAGTCTGAAG |
| | | CCCGAGGACACTGCGATGTATTACTGTAAGGCCTCTTGCGTTCGCGGTCGCACA |
| | | ATTTCCGAGTACTGGGGCCAGGGAACACAGGTGACGGTGAGTAGC |
| DR594- | 1125 | CAGGTACAGCTCCAGGAGTCTGGCGGAGGTTCAGTGCAGGCTGGCGGTTCTTTG |
| hIL27 | | CGCCTGTCCTGTGTAGCCTCCGCCAGCACATATTGCACTTACGACATGCACTGG |
| Ra_VH | | TATCGCCAGGCTCCGGGTAAGGGCCGCGAGTTTGTTAGCGCCATCGACCCCGAT |
| H14 | | GGAACAACCCGTTATGCTGACTCTGTGAAGGGCCGCTTTACAATTAGTCAGGGA |
| | | ACCGCAAAGAATACTGTTTATTTGCAGATGAATAGCCTGCAACCCGAGGACACC |
| | | GCTATGTATTACTGTAAGACAGTGTGCGTTGTGGGCTCCCGCTGGAGCGACTAT |
| | | TGGGGTCAGGGCACACAGGTTACCGTGTCCAGCGGAGGGGGGAGCCAGGTGCAG |
| | | CTCCAGGAGTCAGGTGGGGGATCTGTCCAAGCTGGAGGTTCCCTGCGGTTGTCT |
| | | TGTGTGGCCTCTGGTTATGTGTCATGCGATTATTTCTTGCCCAGCTGGTATCGC |
| | | CAGGCTCCGGGCAAGGAGCGCGAATTTGTGTCTATCATTGACGGCACCGGCTCC |
| | | ACCTCCTACGCAGCCAGTGTCAAGGGGCGCTTCACGGCAAGCCAAGACAAGGGA |
| | | AAGAACATCGCCTATTTGCAGATGAACTCTTTGAAGCCAGAGGACACCGCCATG |
| | | TATTACTGTAAGGCATCTTGTGTGAGGGGCCGCGCGATCAGCGAGTACTGGGGC |
| | | CAGGGCACCCAGGTGACCGTGAGCAGC |
| DR594- | 1126 | CAGGTGCAGGTTCAGGAGTCCGGCGGGGGCAGCGTGCAAGCCGGGGGATCTCTG |
| hIL27 | | CGCCTCTCCTGTGTCGCGAGCGCAAGCACATACTGCACCTACGATATGCACTGG |
| Ra_VH | | TATCGCCAGGCCCCCGGCAAGGGCAGAGAGTTCGTGAGCGCGATTGATTCCGAT |
| H14 | | GGCACTACCCGCTACGCTGATAGTGTGAAAGGACGGTTCACTATCTCCCAGGGC |
| | | ACTGCCAAAAACACAGTGTACCTCCAGATGAACTCCCTTCAGCCCGAGGACACC |
| | | GCCATGTATTACTGCAAAACTGTGTGCGTGGTAGGGTCTCGCTGGAGCGATTAT |
| | | TGGGGGCAGGGAACCCAGGTGACAGTTTCCTCTGGGGGCAGCGGAGGTTCTGGC |
| | | GGTTCCGGCCAGGTGCAGCTTCAGGAAAGCGGAGGTGGGAGCGTGCAGGCCGGA |
| | | GGCAGCCTCAGGCTGAGCTGCGTGGCCTCCGGTTACGTATCATGCGATTACTTC |
| | | CTCCCCTCCTGGTATCGGCAGGCTCCGGGCAAGGAGCGCGAGTTCGTGAGCATT |
| | | ATCGACGGTACAGGCTCCACCAGCTATGCCGCATCTGTGAAGGGACGTTTCACA |
| | | GCTTCCCAGGACAAGGGTAAAAACATTGCCTACTTGCAGATGAACAGCTTGAAG |
| | | CCGGAAGATACCGCAATGTATTACTGCAAAGCCAGTTGTGTCCGTGGCCGCGCT |
| | | ATCTCCGAGTATTGGGGACAGGGCACACAGGTGACGGTGTCCTCC |
| DR594- | 1127 | CAGGTCCAGGTCCAGGAGAGCGGCGGTGGAAGCGTCCAGGCTGGGGGCTCTCTG |
| hIL27 | | CGTCTGTCTTGTGTAGCCTCTGCCTCCACTTACTGTACTTACGACATGCACTGG |
| Ra_VH | | TATCGCCAGGCCCCTGGCAAGGGCCGCGAGTTCGTTTCCGCCATTGATTCTGAC |
| H15 | | GGTACTACCAGATACGCCGACTCCGTGAAAGGTAGGTTCACGATCAGCCAGGGC |
| | | ACTGCCAAAAACACAGTTTATCTCCAGATGAACTCCCTGCAGCCTGAGGACACT |
| | | GCCATGTATTACTGTAAAACCGTTTGTGTGGTCGGATCTCGGTGGTCTGACTAT |
| | | TGGGGGCAGGGCACCCAGGTGACCGTATCTTCCGGCGGAGGGTCCCAGGTCCAG |
| | | TTGCAGGAGTCTGGCGGAGGCTCCGTACAGGCAGGGGGCTCCCTGCGGCTCTCA |
| | | TGCGTGGCCTCCGGCTACGTGTCTTGCGACTATTTCCTGCCATCTTGGTATAGA |
| | | CAGGCTCCCGGCAAGGAGCGCGAGTTTGTGTCTATTATCGACGGCACCGGCTCA |
| | | ACTTCCTACGCTGCCTCTGTGAAGGGCCGCTTTACCGCCAGCCAGGACAAAGGC |
| | | AAGAACATCGCGTACCTCCAGATGAACACCCTGAAGCCCGAGGACACCGCCATG |
| | | TATTACTGTAAGGCCTCCTGTGTGCGTGGACGTGCCATCTCCGAATACTGGGGC |
| | | CAGGGCACCCAGGTGACCGTAAGCTTCA |
| DR594- | 1128 | CAGGTGCAGGTGCAAGAGAGCGGTGGGGGCAGGGTGCAAGCTGGGGGCAGCCTG |
| hIL27 | | CGCCTCTCCTGCGTCGCGTCTGCTTCCACTTACTGTACTTACGACATGCACTGG |
| Ra_VH | | TATCGCCAGGCCCCTGGCAAGGGCCGCGAGTTCGTGTCTGCCATTGACAGCGAT |
| H15 | | GGAACGACCCGCTACGCGGACAGCGTTAAGGGTCGCTTCACTATCTCTCAGGGC |
| | | ACTGCTAAAAACACTGTGTACCTCCAGATGAACTCTTTGCAGCCCGAGGATACT |
| | | GCCATGTATTACTGTAAGACCGTTTGCGTTGTGGGTTCCCGTTGGTCCGATTAT |
| | | TGGGGCCAGGGCACCCAGGTGACCGTATCAAGGGGGGGGTCCGGGGGCAGCGGA |
| | | GGTTCAGGTCAGGTGCAGTTGCAGGAAAGCGGAGGTGGCAGCGTACAGGCCGGG |
| | | GGCTCCCTGAGGCTGAGCTGCGTGGCGAGTGGCTACGTCAGTTGCGACTACTTC |
| | | TTGCCTTCTTGGTATCGGCAAGCCCCCGGTAAGGAGCGCGAGTTCGTGAGTATC |
| | | ATCGACGGCACTGGTAGCACCTCCTATGCTGCCAGCGTGAAAGGTCGTTTTACC |
| | | GCCTCCCAGGACAAGGGTAAAAACATTGCCTATTTGCAAATGAACACCCTTAAA |
| | | CCTGAGGACACTGCCATGTATTACTGTAAGGCTTCATGTGTGAGAGGTCGTGCT |
| | | ATCTCAGAATACTGGGGCCAGGGCACCCAAGTCACTGTTTCTTCT |
| DR594- | 1129 | CAGGTTCAGCTCCAGGAATCCGGCGGTGGCTCCGTTCAGGCAGGGGGCAGCCTG |
| hIL27 | | CGTCTGTCCTGTGTCGCATCCGCCTCTACCTACTGCACTTACGATATGCACTGG |
| Ra_VH | | TATCGGCAGGCTCCTGGTAAGGGCCGCGAGTTCGTCAGTGCTATTGACTCCGAC |
| H16 | | GGTACAACCCGGTATGCGGATTCTGTGAAAGGAAGATTTACGATCTCCCAAGGG |
| | | ACCGCAAAAAACACCGTGTACCTTCAGATGAACTCTCTCCAGCCCGAAGATACA |
| | | GCGATGTATTACTGCAAGACTGTATGCGTGGTAGGTTCTCGTTGGAGCGACTAC |
| | | TGGGGCCAGGGTACACAAGTCACCGTCTCCAGCGGGGGGGGCAGCCAGGTCCAG |
| | | CTCCAGGAATCTGGCGGAGGTTCCGTGCAGGCCGGAGGTAGCCTTCGCCTCTCT |
| | | TGCCGCGCATCCGGCAGCACCTACTCCAACTACTGTCTGGGCTGGTTCAGGCAG |
| | | ATCACCGGCAAGGAGCGCGAGGGCGTAGCCGTAATAAATTGGGTAGGCGGTATG |
| | | CTGTACTTTGCTGACAGCGTTAAGGGTCGGTTTACGGTGTCCCAGGATCAGGCC |
| | | AAGAACACCGTGTATCTCCAGATGAACTCCCTGAAGCCGGAGGATACCGCTATG |
| | | TACTATTGCGCAGCCGAGTCAGCTAGTTCCTTCAGCTGCGGTGGCTGGCTCACG |
| | | AGACCAGATCGCGTCCCCTACTGGGGACAAGGCACACAGGTTACCGTGTCCTCC |
| DR594- | 1130 | CAGGTCCAGCTTCAGGAGAGCGGGGGAGGCTCCGTGCAAGCTGGAGGTTCCCTC |
| hIL27 | | CGCTTGTCCTGCGTTGCGTCCGCAAGCACCTACTGCACTTATGACATGCACTGG |
| Ra_VH | | TATCGCCAGGCTCCTGGCAAAGGGAGAGAGTTCGTCTCCGCCATTGATAGCGAT |
| H16 | | GGTACAACTAGGTACGCAGATTCCGTTAAAGGTAGGTTTACTATCAGCCAGGGC |
| | | ACGGCCAAAAACACCGTCTATGGCAAATGAACTCTCTCCCAGCCCGAGGATACC |
| | | GCGATGTACTATTGCAAGACCGTGTGTGTAGTGGGCAGCCGCTGGTCAGACTAT |
| | | TGGGGCCAGGGAACGCAGGTGACAGTGTCTAGGGGGGGGTCCGGGGGTTCCGGT |
| | | GGCTCCGGCCAGGTACAGCTCCAGGAGTCAGGCGGAGGGTCCGTGCAAGCCGGG |
| | | GGTTCTCTGCGGCTCTCCTGTCGCGCTTCTGGGTCCACCTATTCCAATTACTGC |
| | | CTGGGCTGGTTCCGTCAAATTACGGGCAAGGAGCGCGAAGGCGTGGCCGTGATA |
| | | AATTGGGTGGGCGGGATGTTGTACTTTGCTGACAGCGTTAAGGGACGTTTTACT |
| | | GTGTCCCAGGACCAGGCCAAGAACACCGTGTACTTGCAGATGAACTCCTTGAAG |
| | | CCAGAGGACACAGCAATGTATTACTGTGCCGCTGAAAGCGCCAGCTCTTTCAGT |
| | | TGTGGGGGCTGGCTGACTAGACCAGACCGTGTCCCTTACTGGGGTCAGGGCACC |
| | | CAGGTTACCGTGTCCTCC |
| DR594- | 1131 | CAAGTCCAGCTCCAGGAGTCCGGCGGTGGCTCCGTCCAGGGGGGCGGATCTCTG |
| hIL27 | | CGCCTGTCCTGTGTCGCCTCCGCTTCCACATACTGCACCTATGACATGCACTGG |
| Ra_VH | | TATCGGCAGGCTCCTGGTAAGGGCCGCGAGTTCGTCAGCGCCATCGACTCCGAC |
| H17 | | GGAACGACCGGGTACGCTGATAGCGTGAAAGGTAGGTTTACCATCTCACAGGGC |
| | | ACCGCCAAAAATACCGTCTACTTGCAGATGAACAGTTTGCAGCCTGAGGACACC |
| | | GCCATGTATTACTGCAAGACTGTGTGTGTAGTGGGGTCACGCTGGTCTGATTAT |
| | | TGGGGCCAGGGTACTCAGGTGACTGTGTCTAGTGGCGGTGGCAGCCAGGTGCAG |
| | | TTGCAGGAGAGTGGAGGCGGTCTCGTCCAGCCAGGAGGCAGTCTGCGCCTGTCA |
| | | TGCGCTGCCTCCGGCTTTACTTTTTCCCTGTCTGGTATGTCATGGGTGCGGCAG |
| | | GCCCCCGGCAAGGGTCTGGAGTGGGTCAGTGCAATCAGCAGTGGAGGGGCCTCT |
| | | ACTTATTACACCGACAGCGTCAAAGGCAGATTCACCATCAGTAGGGATAATGCC |
| | | AAGAACATGCTGTACCTCCAGCTGAACTCTCTGAAGACAGAGGACACGGCTATG |
| | | TACTATTGTGCTAAAGGGGGCAGCGGTTACGGGGATGGTTCCAGAATGACTTCC |
| | | CCGGGATCTCAGGGTACTCAAGTCACCGTTAGTTCT |
| DR594- | 1132 | CAAGTCCAGCTCCAGGAGAGCGGAGGGGGTTCCGTGCAGGCAGGGGGTTCACTT |
| hIL27 | | CGCCTGTCCTGTGTGGCTTCTGCTTCCACCTATTGCACGTATGATATGCACTGG |
| Ra_VH | | TATCGCCAGGCTCCCGGCAAGGGCCGCGAGTTCGTCTTCAGCGATGATTCTGAC |
| H17 | | GGCACCACTCGCTACGCTGACTCTGTGAAGGGCCGCTTTACCATCAGTCAAGGT |
| | | ACTGCGAAGAACACCGTGTACCTTCAGATGAACTCACTGCAACCGGAGGACACC |
| | | GCGATGTACTATTGCAAGACTGTTTGTGTGGTCGGTTCCCGCTGGTCCGATTAC |
| | | TGGGGGCAGGGCACTCAGGTGACGGTCAGTTCCGGCGGGAGTGGGGGGTCCGGG |
| | | GGCTCTGGCCAAGTGCAGTTGCAGGAGAGGGCGGGAGGCCTCGTGCAGCCAGGT |
| | | GGATCTCTGCGGCTGTCTTGTGCCGCTTCCGGGTTCACTTTTTCCCTGTCCGGC |
| | | ATGAGTTGGGTCCGCCAGGCCCCAGGGAAAGGCCTGGAGTGGGTGTCCGCCATC |
| | | TCTAGCGGCGGTGCCTCCACATATTACACTGACAGCGTCAAGGGTCGCTTTACG |
| | | ATTTCCCGCGATAACGCTAAGAACATGGTCTACCTCCAGCTGAACAGCCTGAAG |
| | | ACCGAGGATACTGCCATGTATTACTGTGCCAAAGGGGGTAGCGGCTATGGGGAC |
| | | GCCAGCAGGATGACCAGTCCCGGAAGTCAGGGAACTCAGGTCACCGTTTCTTCC |
| DR594- | 1133 | CAGGTACAGCTTCAGGAGAGCGGGGGAGGCTCTGTGCAGGCCGGAGGCTCTCTG |
| hIL27 | | CGCCTCTCTTGTGTCGCCTCCGCGTCTACCTATTGCACCTACGACATGCACTGG |
| Ra_VH | | TATCGCCAGGCCCCCGGAAAAGGTCGCGAGTTCGTGTCTGCTATCGACTCCGAC |
| H18 | | GGGACCACTCGCTACGCTGACTCCGTAAAAGGACGTTTTACAATTAGTCAGGGT |
| | | ACAGCCAAGAATACAGTATACCTGCAAATGAACTCCTTGCAGCCAGAGGACACC |
| | | GCCATGTATTACTGCAAGACCGTGTGCGTGGTCGGCAGTCGCTGGTCCGACTAC |
| | | TGGGGCCAGGGAACCCAGGTCACGGTCTCAAGCGGAGGGGGATCACAGGTCCAG |
| | | CTGCAAGAATCAGGAGGTGGAAGCGTGCAGGCAGGCGGAAGCCTGAGGCTGTCC |
| | | TGCGTGGCCAGCGGCTACGTGTCCTGCGATTACTTTCTCCCTAGCTGGTATCGC |
| | | CAGGCACCCGGAAAAGAGAGGGAGTTCGTCTCAATTATCGACGGAACTGGAAGC |
| | | ACCTGTTACGCCGCTTCCGTGAAGGGCCGCTTTACTGCATCACAGGATAAGGGC |
| | | AAGAATATCGCCTACCTCCAGATGAACTCACTGAAGCCTGAGGACACTGCTATG |
| | | TATTACTGCAAGGCTTCCTGTGTGCGGGGTCGGGGAATCTCTGAGTACTGGGGT |
| | | CAGGGTACTCAGGTCACCGTCTCTTCT |
| DR594- | 1134 | CAGGTGCAGTTGCAGGAGAGCGGAGGTGGGAGTGTCCAGGCTGGTGGCTCCTTG |
| hIL27 | | CGCCTGAGCTGCGTGGCCTCCGCCTCCACTTATTGCACCTACGACATGCACTGG |
| Ra_VH | | TACAGGCAAGCCCCTGGTAAGGGTAGGGAGTTCGTCAGCGCCATTGATAGCGAT |
| H18 | | GGCACAACCCGTTACGCGGATAGCGTGAAGGGCCGTTTCACAATCTCACAGGGC |
| | | ACCGCCAAGAACACCGTTTATTTGCAGATGAACAGCCTCCAGCCCGAAGATACA |
| | | GCTATGTATTACTGTAAGACCGTGTGTGTGGTTGGTAGCAGATGGTCCGACTAC |
| | | TGGGGTCAAGGCACTCAAGTCACCGTATCCAGGGGGGGATCAGGAGGCAGCGGG |
| | | GGCTCCGGGCAGGTGCAGCTCCAGGAATCAGGGGGTGGCAGCGTGCAGGCAGGG |
| | | GGTTCCTTGCGTCTGAGTTGTGTGGCAAGTGGCTATGTAAGCTGTGACTACTTC |
| | | CTCCCCAGTTGGTATCGCCAGGCTCCCGGCAAGGAGCGGGAGTTCGTGAGTATC |
| | | ATTGATGGCACTGGTAGCACCTCCTACGCAGCGAGCGTCAAGGGTCGTTTTACC |
| | | GCGTCTCAGGACAAGGGAAAAAACATCGCGTACCTCCAGATGAACAGCCTGAAG |
| | | CCTGAGGACACAGCCATGTATTACTGCAAGGCCTCCTGGGTGCGCGGTCGCGGA |
| | | ATTTCCGAGTATTGGGGCCAGGGCACCCAAGTAACAGTAAGCTCT |
| DR594- | 1135 | CAGGTCCAACTTCAAGAGTCTGGCGGTGGGTCCGTCCAGGCCGGGGGTTCCCTC |
| hIL27 | | AGACTGTCATGCGTGGCCTCCGCTTCCACCTATTGTACCTACGACATGCACTGG |
| Ra_VH | | TATCGCCAGGCTCCTGGCAAGGGCCGCGAGTTCGTGTCTGCCATTGATTCAGAC |
| H19 | | GGGACAACCCGCTACGCAGACTCCGTGAAAGGTCGTTTTACCATCTCACAGGGC |
| | | ACTGCAAAAAACACTGTCTATCTGCAAATGAACTCCCTGCAGCCAGAAGACACC |
| | | GCCATGTATTACTGCAAGACCGTATGTGTCGTGGGAAGCCGTTGGTCAGACTAC |
| | | TGGGGGCAGGGCACCCAAGTTACGGTGTCCTCTGGGGGGGGCAGTCAGGTGCAG |
| | | TTGCAGGAGAGCGGGGGCGGTTCTGTGCAAGCTGGCGGTTCCCTGCGCCTGAGC |
| | | TGTCGCGCCTCTGGATCTACGTATAGCAACTACTGCCTGGGCTGGTTCCGCCAG |
| | | ATCACCGGAAAGGAACGTGAGGGAGTGGCCGTCATCAACTGGGTGGGCGGAATG |
| | | CTGTACTTCGCCGATTCTGTCAAGGGCCGGTTTACTGTCTCCCAGGATCAGGCG |
| | | AAGAACACGGTATACGTTCAGATGAACAGCCTGAAACCAGAAGATACCGCTATG |
| | | TATTACTGTGCCGCAGAGAGCGTCTCCAGCTTTTCCTGCGGAGGCTGGCTCACC |
| | | CGCCCAGATCGTGTGCCTTACTGGGGGCAGGGGACCCAGGTGACTGTTTCCTCC |
| DR594- | 1136 | CAAGTCCAGCTCCAGGAGTCCGGCGGGGGTTCCGTCCAGCGGGGGGGCTCTTTG |
| hIL27 | | CGGTTGTCTTGTGTCGCATCAGCCTCCACTTATTGTACCTATGATATGCACTGG |
| Ra_VH | | TATCGCCAGGCTCCGGGGAAGGGCAGGGAATTTGTGTCAGCTATTGATTCCGAC |
| H19 | | GGAACCACACGGTATGCAGATTCTGTGAAAGGCCGCTTCACTATCTCACAGGGT |
| | | ACTGCCAAGAACACCGTCTACTTGCAGATGAACTCCCTCCAGCCAGAGGACACT |
| | | GCCATGTATTACTGCAAGACCGTCTGTGTGGTTGGGAGTAGGTGGAGTGACTAC |
| | | TGGGGCCAGGGCACACAGGTCACGGTATCTTCCGGCGGAAGGGGCGGAAGTGGT |
| | | GGCTCTGGACAAGTGCAGTTGCAGGAGAGCGGGGGGGGTTCCGTGCAGGCAGGC |
| | | GGAAGCCTGAGGCTGAGCTGTCGTGCTTCTGGCTCCACCTATTCCAACTATTGC |
| | | TTGGGTTGGTTTCGCCAAATTACCGGCAAGGAGCGTGAAGGCGTTGCTGTCATC |
| | | AACTGGGTTGGGGGTATGCTCTACTTTGCTGACTCCGTGAAGGGTCGCTTCACA |
| | | GTCAGCCAAGACCAGGCTAAGAACACAGTGTATCTCCAGATGAACTCTCTTAAA |
| | | CCGGAAGACACTGCTATGTATTACTGCGCCGCAGAAAGCGTCAGCTCCTTCAGC |
| | | TGCGGTGGCTGGCTGACCAGGCCAGATCGCGTACCATATTGGGGACAGGGAACC |
| | | CAGGTCACCGTGTCCTCC |
| DR594- | 1137 | CAAGTGCAGGTGCAAGAGAGCGGCGGTGGCTCCGTGCAGGCCGGGGGCTCCCTG |
| hIL27 | | CGCCTGAGCTGTGTGGCAAGCGCCTCAACATACTGCACCTATGACATGCACTGG |
| Ra_VH | | TATCGTCAGGCTCCCGGAAAGGGCAGAGAGTTCGTGTCCGCCATCGACAGCGAT |
| H20 | | GGGACAACCCGCTACGCAGACAGCGTGAAGGGCCGCTTCACTATCTCTCAGGGC |
| | | ACAGCTAAGAACACCGTGTACCTTCAGATGAACTCCTTGCAGCCTGAGGATACC |
| | | GCCATGTACTATTGCAAGACCGTGTGTGTAGTGGGCTCCAGATGGTCTGACTAC |
| | | TGGGGGCAGGGAACACAGGTTACCGTCTCTAGCGGGGGGGGAAGCCAAGTGCAG |
| | | TTGCAGGAAAGCGGGGGGGGGGTGGTCCAGCCAGGGGGATCTCTGAGGCTGTCT |
| | | TGCGCCGCATCAGGTTTTACTTTCTCAAGCTACCCCATGTCCTGGGTCAGACAA |
| | | GCGCCGGGGAAGGGCTTGGAATGGGTGAGCACCATCTCCAGCGGTGGAGACACG |
| | | ACCCTGTACGCCGACAGCGTCAAAGGGCGCTTCACATCTAGCCGCGACAATGCA |
| | | AAAAACACCCTGTACCTCCAGGTTAATTCCCTGAAGACAGAGGACACCGCAATG |
| | | TACTATTGCGCGAAACGGATTGACTGCAACTCCGGTTATTGTTACAAGCGCAGT |
| | | TACTGGGGCCAGGGGACCCAGGTGACCGTGTCTAGC |
| DR594- | 1138 | CAGGTTCAGCTTCAGGAATCTGGAGGGGGATCTGTGCAGGCTGGTGGAAGCCTG |
| hIL27 | | CGTCTCAGCTGCGTGGCCTCTGCCTCCACGTATTGTACCTACGACATGCACTGG |
| Ra_VH | | TACAGGCAGGCTCCAGGGAAGGGCCGGGAGTTTGTGAGCGCTATTGATTCAGAC |
| H20 | | GGCACTACACGGTACGCTGACAGTGTCAAGGGGAGGTTCACCATCAGCCAGGGG |
| | | ACAGCTAAGAATACCGTCTACCTCCAGATGAACTCCCTTCAGCCGGAGGATACC |
| | | GCTATGTATTACTGCAAGACGGTGTGCGTGGTCGGCTCCCGTTGGAGCGACTAC |
| | | TGGGGGCAGGGCACCCAAGTTACCGTGTCCAGCGGAGGTTCAGGCGGTTCTGGT |
| | | GGCTGTGGCCAGGTGCAGCTCCAGGAGAGCGGAGGGGGTCTGGTGCAGCCGGGT |
| | | GGCTCCCTGAGACTTTCCTGCGCAGCTTCCGGCTTTACCTTCTCTAGCTATCCA |
| | | ATGTCTTGGGTGAGACAGGCCCCTGGAAAGGGTCTGGAGTGGGTGTCTACTATT |
| | | TCCTCTGGTGGCGATACAACCCTGTACGCGGATTCTGTCAAAGGTCGTTTCACC |
| | | AGCTCACGCGACAACGCTAAGAACACCTTGTATCTTCAGTTGAACAGCCTGAAG |
| | | ACAGAGGACACTGCGATGTATTACTGTGCAAAGCGCATTGATTGTAATAGCGGC |
| | | TACTGTTACAAGCGTAGCTACTGGGGCCAAGGCACCCAGGTCACGGTCAGCAGC |
| DR594- | 1139 | CAGGTGCAGGTTCAGGAGTCTGGTGGCGGTTCCGTCCAGGCTGGGGGTTCTCTG |
| hIL27 | | AGGCTGTCATGCGTGGCATCTGCGAGCACCTACTGTACTTACGACATGCACTGG |
| Ra_VH | | TATCGTCAGGCCCCCGGTAAGGGTCGGGAGTTCGTGTCCGCCATTGATAGCGAT |
| H21 | | GGGACAACCCGGTACGCCGACTCCGTGAAGGGTCGCTTCACTATCTCACAGGGA |
| | | ACCGCTAAGAACACAGTCTACCTTCAGATGAACTCTTTGCAGCCAGAGGACACC |
| | | GCCATGTATTACTGTAAGACAGTGTGTGTAGTTGGAAGCCGCTGGAGCGACTAC |
| | | TGGGGCCAAGGCACTCAGGTCACCGTCAGCTCTGGCGGAGGCAGCCAGGTGCAG |
| | | TTGCAGGAGAGCGGGGGAGGCTTGGTACAGCCCGGAGGCTCCCTCAGGCTCAGC |
| | | TGTGTGCCTCCGGTTTCACCTTTTCCCTGTCTTCCATGTCCCTGGGTGCGTCAG |
| | | GCACCAGGCAAGGGTCTGGAGTGGGTGTCTGCTATCAGCTCCGGCGGAGCTTCC |
| | | ACGTACTATACAGATTCCGTTAAGGGACGGTTCACTATCTCCCGTGACAACGGG |
| | | AAGAATATGCTGTACCTTCAGGTCAACTCACTGAAGACTGAGGACACCGCCATG |
| | | TATTACTGCGCCAAAGGGGGCTCCGGCTATGGCGACGCGAGCCGTATGACTAGC |
| | | CCTGGAAGCCAAGGCACACAGGTCACCGTTAGCTCC |
| DR594- | 1140 | CAGGTTCAGCTTCAGGAGAGCGGGGGAGGCAGCGTCCAGGCCGGAGGCTCACTG |
| hIL27 | | CGCCTGTCTTGTGTCGCCTCTGCATCTACCTACTGCACTTATGACATGCACTGG |
| Ra_VH | | TATCGCCAAGCGCCGGGTAAAGGACGCGAGTTCGTCTCTGCCATCGACTGCGAC |
| H21 | | GGCACAACTCGCTACGCCGACTCTGTGAAGGGCAGGTTTACCATCAGCCAGGGA |
| | | ACCGCTAAGAACACCGTTTACGTCCAGATGAACTCCCTCCAGCCTGAGGACACC |
| | | GCCATGTATTACTGTAAGACCGTTTGTGTCGTGGGCTCCAGGTGGTCCGACTAC |
| | | TGGGGACAGGGCACCCAGGTGACAGTCTCCAGCGGTGGAAGTGGTGGCTCCGGC |
| | | GGTTCAGGCCAGGTCCAGTTGCAGGAGTCTGGCGGTGGACTCGTGCAACCTGGG |
| | | GGCTCTCTGAGACTGAGCTGTGCGGCTTCTGGGTTCACCTTCAGTCTGAGCAGT |
| | | ATGAGCTGGGTCCGCCAAGCCCCAGGAAAGGGACTGGAATGGGTGTCCGCCATG |
| | | TCCTCTGGCGGAGCGAGCACATACTATACAGATAGCGTGAAAGGGAGGTTTACT |
| | | ATCTCTAGGGATAACGCGAAGAACATGCTTTATCTCCAGCTTAATTCCCTGAAG |
| | | ACTGAGGACACTGCCATGTACTATTGCGCCAAAGGTGGCAGCGGTTATGGAGAC |
| | | GCCAGTAGAATGACCAGCCCAGGTTCCCAGGGGACTCAAGTGACCGTCAGTTCT |
| DR594- | 1141 | CAGGTGCAGCTTCAGGAAAGCGGAGGCGGTAGCGTACAGGCCGGGGGTAGCCTG |
| hIL27 | | CGTTTGTCCTGCGTGGCGAGCGCTTCCACGTACTGTACCTACGACATGCACTGG |
| Ra_VH | | TATCGCCAAGCGCCGGGAAAGGGACGCGAGTTCGTTTCCGCTATTGATTCCGAT |
| H22 | | GGGACCACACGCTACGCAGATTCCGTGAAGGGCCGCTTCACAATTTGCCAGGGC |
| | | ACAGCCAAGAACACCGTGTACCTCCAGATGAACTCCTTGCAGCCTGAGGATACC |
| | | GCCATGTATTACTGCAAAACAGTCTGCGTCGTGGGTTCTCGTTGGTCTGACTAT |
| | | TGGGGCCAGGGAACTCAGGTTACCGTTTCCTCTGGGGGGGGGTCCCAGGTCCAG |
| | | CTCCAGGAGTCAGGTGGGGGGTCCGTCCAAGCAGGTGGCTCCCTGCGCCTGTCA |
| | | TGTCGGGCGAGCGGCAGTACTTATAGCAATTACTGTCTGGGCTGGTTCCGGCAG |
| | | ACAACCGGCAAAGAGAGAGAGGGCGTTGCCGTTATTAACTGGCGGCGGGGAATG |
| | | CTGTACTTCGCCGACTCCGTAAAGGGACGCTTCACCGTTAGCCAGGATCAGGCT |
| | | AAAAATACAGTATACCTGCAAATGAACAGTTTGAAGCCTGAAGACACAGCCATG |
| | | TACTATTGCGCCGCTGAGTCTGTCAGCTCTTTTTCTTGGGGGGGATGGCTGACT |
| | | CGCCCGGATCGCGTACCTTATTGGGGCCAGGGAACCCAAGTCACGGTCTCTAGT |
| DR594- | 1142 | CAAGTGCAGCTTCAGGAAAGCGGAGGTGGAAGTGTGCAGGCTGGGGGTAGCCTG |
| hIL27 | | AGGCTGTCCTGCGTGGCGAGCGCGTCCACTTACTGTACCTACGACATGCACTGG |
| Ra_VH | | TATCGCCAAGCACCAGGCAAGGGTCGCGAGTTCGTTTCCGCCATTGATAGCGAT |
| H22 | | GGCACCACTCGTTACGCTGACAGTGTGAAGGGTCGCTTTACCATTTCCCAGGGC |
| | | ACAGCGAAGAACACCGTTTACCTCCAGATGAACAGCCTTCAGCCAGAGGATACA |
| | | GCCATGTACTATTGCAAAACCGTGTGCGTTGTGGGCAGCCGTTGGTCTGACTAT |
| | | TGGGGACAAGGCACTCAGGTTACTGTCAGCTCCGGTGGGTCCGGGGGATCTGGG |
| | | GGCAGCGGCCAGGTCCAGCTGCAAGAGTCTGGTGGGGGTTCCGTCCAGGGGGGC |
| | | GGGAGTTTGCGGCTCAGCTGCCGCGCCTCAGGGTCAACTTACAGTAACTATTGC |
| | | CTGGGCTGGTTCAGGCAGACCACAGGCAAGGAGAGAGAGGGTGTCGCCGTCATC |
| | | AACTGGGTAGGCGGTATGCTGTACTTCGCCGACTCCGTTAAAGGGCGTTTCACG |
| | | GTTTCCCAAGACCAGGCAAAGAACACTGTGTATCTCCAGATGAACAGCCTCAAA |
| | | CCCGAAGATACCGCGATGTATTACTGTGCTGCCGAGAGCGTGTCTTCCTTCAGC |
| | | TGTGGCGGTTGGCTTACACGCCCGGATCGGGTTCCTTACTGGGGCCAGGGCACA |
| | | CAGGTGACAGTCTCCAGG |
| DR594- | 1143 | CAGGTGCAGCTCCAGGAGAGGGGGCGAGGCTCTGTGCAGGCCGGTGGCTCACTT |
| hIL27 | | CGCCTGTCCTGCGTGGCTTCAGCCAGCACTTACTGTACCTATGATATGCACTGG |
| Ra_VH | | TATCGCCAGGCCCCTGGCAAAGGACGCGAGTTCGTGTCTGCCATTGATAGTGAT |
| H23 | | GGCACCACACGCTACGCCGACTCAGTTAAGGGTAGGTTTACCATTTCCCAAGGC |
| | | ACGGCCAAGAACACCGTGTACCTGCAAATGAACTCCCTCCAGCCCGAGGACACA |
| | | GCGATGTATTACTGCAAGACCGTGTGTGTTGTGGGTTCCCGCTGGTCCGACTAT |
| | | TGGGGTCAGGGCACGCAGGTGACCGTTAGTTCCGGTGGAGGCAGCCAGGTCCAG |
| | | CTTCAGGAGTCAGGCGGGGGCTCAGTCCAGGCTGGCGGATCTCTCCGCTTGTCC |
| | | TGTAGGGCCTCCCGTAGCCCCTACGGCAACTATTGTTTGGGTTGGTTCCGCCAG |
| | | TCCACTGGCAAGGAGCGGGAGGGAGTTGCGGTCATCAACTGGGTGGGGGGGATG |
| | | CTCTACTTTGCGGACTCCGTGAAAGGCCGCTTCACCGTTAGCCAGGATCACGCT |
| | | AAGAACACTGTGACACTCCAGATGAACAGCCTGAAGCCTGAGGATACTGCCATG |
| | | TACTATTGCGCCGCAGAGAGCGTGTCTAGCTTCTCCTGCGGCGGTTGGCTGACC |
| | | AGACCCGACAGGGTTCCGTATTGGGGTCAAGGCACCCAGGTGACTGTCTCTAGC |
| DR594- | 1144 | CAGGTTCAGCTTCAGGAGAGCGGGGGCGGTTTCTGTCAGGCAGGTGGGTCCCTG |
| hIL27 | | AGACTGTCTTGCGTTGCCAGCGCCTCCACGTATTGCACCTATGACATGCACTGG |
| Ra_VH | | TATCGCCAGGCCCCTGGCAAGGGCAGAGAGTTCGTTTCTGCCATTGATTCTGAT |
| H23 | | GGCACAACCCGCTACGCCGATTCTGTCAAGGGACGCTTCACGATTTCCCAGGGG |
| | | ACCGCCAAGAACACGGTATATCTGCAAATGAACTCTCTCCAGCCTGAAGACACA |
| | | GCGATGTACTATTGTAAAACCGTGTGCGTCGTAGGCAGTCGTTGGTCCGATTAC |
| | | TGGGCCAGGGCACTCAAGTTGACTGTCAGCTCCGGGGGCTCTGGAGGGAGCGGT |
| | | GGCTCTGGTCAGGTCCAACTGCAAGAGTCAGGGGGGGGCTCCGTGCAGGCCGGA |
| | | GGCTCCCTGCGCCTTTCCTGTCGCGCAAGCCGTTCTCCATACGGGAACTATTGT |
| | | CTGGGCTGGTTCAGACAGAGTACTGGGAAGGAGAGGGAGGGCGTGGCCGTCATC |
| | | AACTGGGTCGGTGGAATGCTGTACTTCGCTGACAGTGTGAAGGGCCGTTTCACT |
| | | GTGTCCCAGGATCACGCCAAAAACACTGTTACACTCCAGATGAACAGCCTGAAG |
| | | CCAGAAGATACAGCAATGTATTACTGTGCGGCTGAATCTGTTTCCAGCTTTTCC |
| | | TGCGGCGGTTGGCTGACGCGCCCTGATCGCGTCCCCTATTGGGGCCAGGGTACT |
| | | CAGGTTACTGTGTCATCT |
| DR594- | 1145 | CAGGTTCAGCTGCAAGAGTCTGGCGGAGGCTCTGTGCAGGCAGGAGGCTCTCTC |
| hIL27 | | CGGCTGAGCTGCGTGGCCTCCGCCTCCACTTACTGTACTTACGATATGCACTGG |
| Ra_VH | | TATCGCCAAGCGCCGGGGAAGGGCCGTGAGTTTGTTAGCGCCATCGACTCCGAC |
| H24 | | GGTACGACTCGCTACGCCGATTCTGTGAAAGGGCGCTTCACTATTTCCCAAGGC |
| | | ACCGCCAAAAACACCGTGTACCTTCAGATGAACTCTCTTCAGCCAGAGGACACC |
| | | GCCATGTATTACTGCAAGACAGTCTGTGTTGTGGGCAGTCGTTGGAGCGATTAC |
| | | TGGGGTCAGGGTACTCAGGTTACTGTCAGCTCCGGGGGGGGAAGCCAGGTCCAG |
| | | CTCCAGGAGAGCGGGGGGGGCCTGGTGCAACCTGGCGGTTCTCTGCGCCTTTCC |
| | | TGCGCTGCGTCTGGGTTCACATTCTCCCACTCCGGCATGAGCTGGGTCCGTCAG |
| | | GCTCCGGGGAAGGGCCTTGAGTGGGTGTCCACGATCAATAGTGGCGGAGCCAGC |
| | | ACCTATTACACCGACTCTGTCAAGGGCCGGTTCACTATCAGCCGGGATAACGCC |
| | | AAGAACATGGTGTATTTGCAGGTGAACTCTCTGAAGACGGAGGACACCGCGATG |
| | | TATTACTGTGCCAAGGGAGGCTCCGGCTATGGGGATGCCTCCAGGATGACTTCT |
| | | CCGGGTAGCCAGGGAACACAGGTGACAGTCTCAAGC |
| DR594- | 1146 | CAGGTCCAACTGCAAGAGTCTGGAGGGGGTAGCGTCCAGGCCGGGGGTTCCCTG |
| hIL27 | | CGGCTGTCCTGTGTCGCCTCTGCGAGCACATACTGTACCTATGATATGCACTGG |
| Ra_VH | | TATCGCCAAGCGCCGGGGAAGGGACGCGAGTTCGTCAGCGCCATTGATTCAGAC |
| H24 | | GGAACGACTCGGTACGCGGACTCTGTCAAGGGACGGTTCACCATCTCACAGGGT |
| | | ACAGCCAAGAATACAGTCTACTTGCAGATGAACTCCTTGCAGCCCGAGGACACA |
| | | GCCATGTATTACTGTAAAACCGTTTGCGTAGTGGGCTCCAGGTGGAGCGATTAC |
| | | TGGGGCCAGGGCACTCAGGTCACCGTCAGCTCGGGCGGTTCCGGGGGGAGCGGA |
| | | GGCAGCGGTCAGGTACAGCTGCAAGAAAGCGGAGGTGGGCTGGTTCAGCCGGGA |
| | | GGCAGCCTGCGCCTGTCCTGCGCCGCGTCAGGTTTCACGTTCTCTCACTGTGGG |
| | | ATGAGCTGGGTGCGGCAAGCGCCGGGCAAGGGCCTCGAATGGGTCTCTACCATC |
| | | AATTCTGGGGGGGCCTCCACCTATTACACCGATAGCGTTAAAGGCAGATTCACC |
| | | ATCTCACGTGACAATGCCAAGAATATGCTGTACTTGCAGCTCAACAGTCTGAAG |
| | | ACCGAAGATACTGCTATGTATTACTGTGCCAAGGGGGGGAGCGGATATGGTGAC |
| | | GCATCCCGTATGACCAGCCCTGGCTCCCAGGGCACCCAGGTGACCGTCTCTAGC |
| DR595- | 1147 | CAAGTCCAGCTCCAGGAGAGCGGGGGAGGCTCTGTTCAGGCTGGGGGCAGTCTG |
| hIL27 | | ACATTGTCCTGCGCTGCCAGCGAGTACGCCTATTCCACCTGTAACATGGGATGG |
| Ra_VH | | TATCGGCAAGCCCCAGGTAAGGAACGCGAACTCGTCTCAGCTTTCATCAGCGAT |
| H1 | | GGCAGCACTTATTACGCCGACTCTGTGAAGGGCCGCTTTACCATCACAAGGGAT |
| | | AATGCAAAGAATACAGTGTATCTGCAAATGAACTCCCTCAAGCCGGAGGACACA |
| | | GCTATCTATTACTGTTCCGCGAACTGCTACAGGCGTCTGAGGAACTACTGGGGG |
| | | CAGGGCACTCAGGTTACCGTTAGCTCCGGCGGTGGAAGCCAGGTGCAGCTCCAG |
| | | GAGTCTGGAGGCGGGCTGGTGCAGCCTGGGGGCAGCCTTCGCCTGTCTTGCGCG |
| | | GCCAGCGGATTCACCTTCTCTAGCTACCCAATGTCTTGGGTGAGGCAGGGTCCT |
| | | GGCAAAGGCCTTGAGTGGATCTCCACAATCAGCGCCGGAGGTGATACCACTCTG |
| | | TACGCAGACAGTGTGAAGGGCCGCTTCACATCTTCCCGCGACAATGCCAAGAAC |
| | | ACCCTGTACCTCCAGCTGAACTCACTGAAGACCGAGGACGCAGCGATCTACTAT |
| | | TGTGCTAAACGTATTGACTGCAACAGTGGGTATTGCTACCGCCGTAACTACTGG |
| | | GGGCAAGGGACGCAGGTCACCGTCTCTTCC |
| DR595- | 1148 | CAGGTGCAGCTCCAGGAGTCTGGTGGGGGTTCTGTGCAGGCAGGGGGCTCCCTT |
| hIL27 | | ACACTGAGCTGCGCCGCGTCAGAGTATGCGTACAGCACGTGCAACATGGGCTGG |
| Ra_VH | | TATCGCCAAGCCCCAGGTAAGGAGCGTGAGCTGGTGTCCGCGTTCATCTCCGAT |
| H1 | | GGCTCTACTTATTACGCTGACTCCGTCAAGGGACGCTTCACAATCACACGTGAC |
| | | AACGCGAAGAACACCGTGTACCTTCAGATGAACTCTCTGAAACCAGAAGACACA |
| | | GCGATCTACTATTGCTCTGCTAACTGCTACCGCAGGCTGCGCAACTACTGGGGT |
| | | CAAGGCACCCAGGTCACCGTCAGCTCCGGTGGGAGTGGTGGCAGCGGTGGCTCC |
| | | GGCCAGGTGCAGCTCCAGGAGTCTGGGGGGGGGCTGGTGCAGCCAGGTGGCTCC |
| | | CTTAGATTGTCTTGCGCAGCTAGTGGATTTACCTTTTCTTCCTACCCAATGTCC |
| | | TGGGTTCGCCAAGCTCCCGGCAAGGGGCTGGAATGGATTAGTACCATCTCTGCG |
| | | GGAGGCGATACTACACTGTATGCCGATTCCGTCAAAGGCCGGTTCACCTCTTCC |
| | | AGAGACAACGCCAAGAACACCCTGTATTTGCAGCTGAACAGCCTGAAGACCGAG |
| | | GATGCTGCCATCTACTATTGCGCTAAGCGTATTGATTGCAACAGCGGCTACTGT |
| | | TATCGGCGCAACTACTGGGGCCAGGGCACCCAGGTTACTGTTAGCAGT |
| DR595- | 1149 | CAGGTTCAGCTCCAGGAAAGCGGTGGGGGGAGCGTGCAGGCCGGAGGCTCTCTG |
| hIL27 | | ACTCTGTCCTGTGCTGCGAGCGAATACGCCTACTCAACTTGCAACATGGGCTGG |
| Ra_VH | | TATCGCCAGGCACCCGGCAAAGAGCGCGAGTTGGTGAGTGCCTTCATCTCCGAT |
| H2 | | GGCAGCACCTATTACGCAGATTCCGTTAAGGGGCGCTTCACAATCACCCGCGAC |
| | | AACGCGAAGAACACCGTGTACCTCCAGATGAACTCTCTGAAGCCCGAGGATACC |
| | | GCCATTTATTACTGTTCTGCTAACTGTTACAGACGCCTTCGTAATTATTGGGGC |
| | | CAGGGCACCCAGGTAACCGTTAGCTCTGGGGGGGGCAGCCAGGTGCAGTTGCAG |
| | | GAGTCCGGGGGGGGACTGGTGCAGCCTGGAGGGTCTCTTAGGCTGTCTTGTGCC |
| | | GCGTCCGGGTTCACCTTTTCCCCTCCCGGCATGTCATGGGTTAGACAGGCACCA |
| | | GGCAAGGGCGTTGAGTGGGTATCTGCCATCTCATCCGGGGCGGCGTCTACCTAC |
| | | TATACCGATTCCGTTAAAGGCCGCTTCACCATTTCTAGGGACAATGCAAAGAAT |
| | | ATCCTGTACCTCCAGTTGAACAGTCTGAAGACCGAGGATACAGCCATGTACTAT |
| | | TGTGCAAAAGGGGGCTCCGGCTACGGCGACGCCAGCCGCATGACATCTCCAGGG |
| | | TCTCAAGGCACCCAAGTGACCGTATCTAGC |
| DR595- | 1150 | CAGGTCCAGCTGCAAGAAAGCGGTGGGGGCAGCGTACAGGCTGGGGGATCTCTG |
| hIL27 | | ACCCTGAGCTGTGCTGCCTCAGAGTACGCATATTCAACCTGCAATATGGGCTGG |
| Ra_VH | | TATCGCCAAGCCCCCGGAAAGGAGCGCGAACTCGTGTCCGCTTTCATCAGTGAC |
| H2 | | GGGTCCACGTACTATGCCGATTCCGTCAAAGGCCGTTTTACTATCACACGCGAC |
| | | AACGCCAAGAATACCGTCTACCTCCAGATGAACAGCCTGAAGCCAGAAGATACA |
| | | GCCATCTATTACTGTAGTGCGAACTGCTACAGACGCCTGCGCAACTAGCGGGGA |
| | | CAGGGCACCCAAGTGACTGTGTCCTCTGGAGGCAGCGGGGGATCTGGAGGGAGT |
| | | GGACAGGTTCAGCTTCAGGAGAGCGGGGGCGGACTGGTGCAGCCAGGAGGCTCC |
| | | CTGAGGTTGTCATGCGCGGCCAGCGGTTTCACGTTCAGTTTGTCCGGCATGTCA |
| | | TGGGTACGCCAAGCGCCCGGTAAGGGTCTGGAATGGGTGTCCGCTATTTCTAGC |
| | | GGAGGCGCGAGTACCTATTACACCGACAGCGTGAAGGGGCGCTTTACTATCTCT |
| | | CGTGATAACGCCAAGAACATTCTCTATCTCCAACTTAATTCTCTGAAGACCGAG |
| | | GACACCGCGATGTATTACTGCGCCAAGGGCGGTTCTGGTTATGGCGACGGTTCC |
| | | CGCATGACATCTCCGGGTAGTCAGGGCACACAGGTGACTGTGTCTAGC |
| DR595- | 1151 | CAGGTGCAGCTCCAGGAGTCCGGCGGAGGCAGCGTGCAAGCCGGTGGCTCCCTC |
| hIL27 | | ACCCTGTCATGCGCGGCCAGCGAGTACGCCTATAGCACCTGTAACATGGGCTGG |
| Ra_VH | | TACAGGCAAGCGCCGGGTAAGGAGCGTGAACTCGTCTCTGCCTTTATCTCCGAT |
| H3 | | GGGTCCACCTATTACGCAGACAGCGTGAAGGGCCGGTTTACGATCACCCGCGAC |
| | | AACGCCAAAAACACAGTCTATCTCCAGATGAACAGTCTGAAACCCGAGGATACG |
| | | GCAATTTACTATTGTAGCGCTAACTGCTACCGTAGACTCAGAAACTACTGGGGC |
| | | CAGGGCACACAGGTGACCGTGAGTTCCGGGGCGGGGTCTCAGGTGCAACTCCAA |
| | | GAGTCCGGGGGGGGCTCCGTCCAGGCTGGCGGTAGCCTGCGCTTGTCTTGTGTG |
| | | GCATCTGGATACGTATCTTGCGACCTATTTCCTCCCCCCTGGTATCGTCAAGCT |
| | | CCGGGAAAAGAGCGGGAGTTCGTGTCCATTATCGACGGGACCGGCTCCACGAGC |
| | | TATGGTGCCTCTGTGAAGGGAAGATTTACTGCCAGTGAGGACAAGGGTAAGAAT |
| | | ATCGGTTATCTGCAAATGAATAGTCTGAAGCCGGAAGACACTGCGATGTATTAC |
| | | TGCAAGGCGAGTTGCGTCAGAGGACGCGCCGTATCCGAATACTGGGGGCAGGGT |
| | | ACGCAGGTGACGGTCTCATCT |
| DR595- | 1152 | CAAGTCCAGCTTCAAGAGAGTGGCGGAGGCTCCGTTCAGGCCGGTGGCTCTCTG |
| hIL27 | | ACTTTGTCCTGCGCCGCAAGCGAGTACGCATACTCTACCTGCAATATGGGCTGG |
| Ra_VH | | TATAGACAAGCGCCTGGCAAGGAGCGCGAGCTGGTATCAGCCTTCATCAGCGAC |
| H3 | | GGATCTACCTACTATGCGGATTCAGTGAAGGGGAGGTTCACCATCACACGCGAC |
| | | AACGCGAAGAATACTGTGTACCTTCAGATGAACAGCCTGAAACCCGAGGACACC |
| | | GCAATCTATTACTGCTCTGCCAACTGTTACAGGCGGCTGCGCAATTACTGGGGC |
| | | CAAGGAACGCAAGTGACAGTATCCAGCGGGGGGAGTGGCGGTAGTGGGGGATCT |
| | | GGCCAGGTGCAGCTCCAGGAGAGCGGCGGAGGGTCCGTACAGGGGGGCGGCAGT |
| | | TTGCGCCTGTCCTGTGTCGCTAGTGGTTACGTGTCCCGCGACTATTTTTTGCCC |
| | | TCTTGGTATCGGCAGGCTCCTGGGAAGGAACGTGAGTTCGTAAGCATCATTGAT |
| | | GGGACCGGCTCCACCTCCTACGCTGCCAGCGTCAAGGGGAGATTTACTGCATCC |
| | | GAAGATAAAGGCAAAAACATTGCTTACCTCCAGATGAACTCCCTCAAACCAGAG |
| | | GATACCGCCATGTATTACTGCAAGGCCTCCTGTGTGAGGGGCCGCGCCGTGTCT |
| | | GAGTACTGGGGTCAGGGCACTCAGGTGACTGTGTCCTCC |
| DR595- | 1153 | CAGGTGCAGGTTCAGGAATCCGGCGGAGGCAGCGTCCAAGGGGGAGGCAGCCTG |
| hIL27 | | ACCCTGTCTTGCGCTGCCAGTGAGTATGCTTACTCCACTTGTAACATGGGCTGG |
| Ra_VH | | TATCGTCAAGCGCCAGGGAAGGAGCGTGAGCTGGTCTCTGCCTTCATCTCAGAT |
| H4 | | GGTTCCACCTATTACGCTGACAGTGTCAAGGGCAGGTTCACCATCACTCGGGAT |
| | | AATGCCAAGAACACCGTGTACCTCCAGATGAACTCCCTGAAGCCTGAAGACACT |
| | | GCCATCTATTACTGCTCCGCCAACTGCTACCGTCGCCTGAGAAATTACTGGGGT |
| | | CAGGGAACCCAGGTCACCGTGTCTTCCGGGGGAGGCTCCCAGGTCCAGCTGCAA |
| | | GAGTCTGGTGGCGGTTTGGTGCAGCCTGGCGAGTCCCTGCGCCTGTCTTGCACC |
| | | GCGAGCGGCTTCACCTTCAGCAATTACGCTATGAGCTGGGTCCGTCAGGCTCCT |
| | | GGGAAGGGCCTGGAGTGGGTAAGTGGAATCAACGTGGCCTACGGTATCACGTCC |
| | | TATGCTGATAGCGTGAAGGGCAGATTTACCATCTCTCGGGACAACACCAAGAAC |
| | | ACACTGTACCTCCAACTCAACAGCCTGAAGACCGAAGACACGGCTATCTATTAC |
| | | TGTGTCAAGCACAGTGGCACCACGATCCCCAGAGGCTTTATTAGTTATACTAAA |
| | | CGGGGTCAGGGGACCCAGGTGACCGTCAGCTCC |
| DR595- | 1154 | CAAGTTCAGCTTCAGGAAAGCGGGGGAGGCAGCGTCCAGGCCGGTGGAAGTTTG |
| hIL27 | | ACGCTGAGCTGTGCGGCTAGTGAATACGCATATTCCACCTGCAATATGGGCGGG |
| Ra_VH | | TACAGGCAGGCCCCCGGCAAGGAACGCGAACTGGTGAGTGCTTTTATCAGCGAC |
| H4 | | GGCTCCACGTATTACGCAGATTCCGTGAAAGGAAGGTTTACCATTACGCGCGAT |
| | | AACGCCAAGAACACTGTGTACCTTCAGATGAACTCTCTCAAACCCGAGGACACT |
| | | GCCATCTACTATTGCTCAGCCAACTGTTACCGCAGGTTGCGTAACTACTGGGGC |
| | | CAGGGCACCCAGGTCACAGTCAGCTCCGGGGGGAGTGGTGGATCTGGCGGTTCC |
| | | GGGCAAGTGCAACTTCAGGAATCCGGCGGGGGCCTGGTGCAGCCTGGGGAGTCC |
| | | CTGCGCCTTAGCTGTACTGCTTCCGGCTTCACGTTTTCCAACTACGCCATGAGT |
| | | TGGGTGCGCCAGGCTCCTGGCAAGGGCTTGGAGTGGGTGTCTGGGATTAACGTG |
| | | GCTTACGGGATTACCTCTTATGCCGACTCAGTCAAGGGTCGTTTCACAATCAGT |
| | | CGCGACAACACCAAAAACACTCTGTATGTGCAACTGAATAGTCTGAAGACCGAG |
| | | GATACCGCTATCTATTACTGCGTCAAGCACTCCGGTACTACCATCCCCAGGGGC |
| | | TTTATTAGTTACACGAAACGGGGTCAGGGGACTCAGGTGACAGTGTCCTCC |
| DR595- | 1155 | CAGGTGCAGCTGCAAGAATCCGGCGGAGGCCCCGTGCAGGCAGGTGGCAGTCTG |
| hIL27 | | ACACTGAGCTGCGCCGCTTCCGAGTATGCCTATTCCACCTGCAACATGGGTTGG |
| Ra_VH | | TACAGGCAGGCTCCTGGTAAGGAACGCGAATTGGTGTCCGCCTTCATTTCCGAT |
| H5 | | GGTAGCACCTACTATGCCGACAGTTGTAAAGGGGCGGTCACCATCACTCGGGAC |
| | | AACGCAAAAAATACCGTGTACCTGCAAATGAACTCCTTGAAGCCAGAGGACACC |
| | | GCTATCTATTACTGCTCCGCAAACTGCTATCGCAGGCTGAGGAACTATTGGGGC |
| | | CAGGGCACCCAGGTGACGGTCTCCTCTGGGGGTGGCTCCCAAGTCCAGCTCCAG |
| | | GAGTCAGGAGGTGGGAGTGTGCAAGCCGGGGGAAGCCTGCGTCTGTCCTGTACC |
| | | GCCAGCGGCTACGTGAGCTGTGACTATTTCCTCCCCTCATGGTATAGACAGGCC |
| | | CCTGGGAAGGAGAGAGAATTTGTTAGCGTGATCGACGGCACCGGGTCCACCAGT |
| | | TACGGTGCCAGCGTCAAAGGGCGCTTTACCGCTTCTCAGGACAAGGGGAAGAAC |
| | | ATTGCCTACCTGCAAATGAACAGCCTGAAACCCGAGGACACTGCAATGTATTAC |
| | | TGTAAGGCCAGTTGTGTGCGTGGCCGTGCCATTAGCGAATATTGGGGCCAGGGG |
| | | ACCCAGGTGACCGTGTCTAGT |
| DR595- | 1156 | CAGGTCCAGGTCCAGGAGAGCGGAGGTGGCAGTGTGCAGGCTGGTGGCTCCCTG |
| hIL27 | | ACCCTTAGTTGTGCAGCCAGCGAATATGCTTACTCCACCTGTAACATGGGATGG |
| Ra_VH | | TATCGTCAGGCTCCGGGCAAGGAGCGGGAGCTTGTGAGCGCATTTATCAGCGAC |
| H5 | | GGGTCAACCTATTACGCAGATTCCGTGAAAGGGCGCTTCACCATCACCCGTGAC |
| | | AACGGTAAGAACACCGTCTACCTCCAGATGAACTCACTGAAGCCCGAGGACACC |
| | | GCTATCTATTACTGTTCTGCCAACTGCTACAGACGCCTCAGGAATTACTGGGGA |
| | | CAGGGCACGCAGGTCACTGTTAGCTCCGGTGGCAGTGGTGGCAGGGGGGGCAGT |
| | | GGACAGGTGCAGTTGCAGGAATCCGGGGGGGGAGTTGTTCAGGCAGGGGGATCT |
| | | CTGCGCCTGAGCTGCACCGCCTCTGGATATGTGAGCTGTGATTACTTCCTGCCT |
| | | AGCTGGTATCGGCAGGCCCCCGGCAAGGAACGTGAGTTCGTGAGCGTCATCGAC |
| | | GGCACCGGCTCCACCTCTTACGCAGCCTCTGTGAAGGGCCGCTTCACAGCCAGC |
| | | CAAGACAAGGGGAAGAATATCGCCTATCTCCAGATGAACAGCCTGAAACCCGAG |
| | | GATACAGCCATGTATTACTGTAAAGCCAGCTGTGTGCGCGGCAGAGCTATCTCC |
| | | GAGTACTGGGGCCAGGGCACCCAGGTTACCGTCTCCCCC |
| DR595- | 1157 | CAGGTCCAACTCCAGGAGAGCGGAGGGGGCTCTGTTCAGGCCGGAGGCTCCCTG |
| hIL27 | | ACCCTCTCCTGCGCAGCCAGTGAATATGCTTACTCCACTTGTAACATGGGATGG |
| Ra_VH | | TATCGTCAAGCCCCTGGTAAAGAACGTGAGCTGGTCTCCGCCTTTATCTCCGAC |
| H6 | | GGTTCCACTTATTACGCCGATAGTGTCAAGGGCCGCTTTACCATCACGAGGGAC |
| | | AATGCCAAGAATACCGTCTACCTCCAGATGAACTCTCTGAAGCCTGAGGATACT |
| | | GCCATCTACTATTGTAGTGCAAACTGCTACCGGAGATTGAGAAATTATTGGGGT |
| | | CAGGGCACCCAGGTGACAGTGTCCAGCGGTGGGGGGAGTCAAGTGCAGTTGCAG |
| | | GAGTCTGGTGGCGGACTGGTGCAGCCGGGGGGAAGTCTGCGTTTGTCTTGCGCC |
| | | GCGAGCGGTTTCAGTTTTAGCAGTTACGCCATGAAATGGGTGCGTCAGGCCCCT |
| | | GGGAAGGGCCTGGAGTGGGTGAGTACCATCTCCAGCGGTGGCAGCAGTACTAAC |
| | | TACGCCGACTCTGTGAAAGGCCGCTTCACAATCTCACGTGATAACGCCAAAAAT |
| | | ACCCTCTACCTTCAGCTCAACTCCCTGAAAATCGAGGACACTGCCATGTACTAT |
| | | TGTGCTAAGGCCATTGTGCCGACTGGTGCCACTATGGAACGTGGGCAGGGCACT |
| | | CAGGTGACCGTTTCTTCC |
| DR595- | 1158 | CAGGTGCAGGTCCAGGAAAGCGGTGGAGGCTCCGTGCAGGCTGGGGGTTCCCTG |
| hIL27 | | ACGCTGTCTTGTGCAGCCTCCGAGTACGCCTATAGCACCTGCAACATGGGCTGG |
| Ra_VH | | TATCGCCAGGCCCCCGGCAAGGAACGTGAACTGGTATCCGCGTTCATCCCTGAT |
| H6 | | GGCAGTACCTACTATGCGGATTCTGTGAAGGGCCGCTTCACCATCACAAGAGAC |
| | | AACGCTAAGAACACCGTGTATCTCCAAATGAACTCCCTCAAGCCCGAGGATACA |
| | | GCAATCTACTATTGCAGCGCTAACTGCTACAGACGCCTGAGGAACTATTGGGGA |
| | | CAGGGGACCCAGGTGACAGTTTCTAGTGGAGGCAGCGGAGGCAGCGGAGGGTCT |
| | | GGCCAGGTTCAGTTGCAGGAGAGTGGGGGGGGTTTGGTGCAGCCAGGGGGCTCC |
| | | CTGAGGCTCAGTTGCGCTGCCTCTGGATTCTCCTTCTCCTCTTATGCCATGAAG |
| | | TGGGTGCGCCAAGCGCCCGGCAAGGGCGTGGAGTGGGTATCTACCATCAGCTCT |
| | | GGGGGCTCCAGCACGAATTACGCAGACAGCGTGAAGGGACGCTTTACTATTTCA |
| | | AGAGATAATGCCAAGAACACTCTGTATCTTCAGCTCAACTCCCTGAAGATCGAG |
| | | GATACCGCTATGTACTATTGCGCCAAAGCCATCGTCCCCACTGGGGCAACAATG |
| | | GAACGCGGCCAAGGAACACAGGTCACCGTCAGTTCT |
| DR595- | 1159 | CAGGTGCAGTTGCAGGAGAGCGGCGGTGGCTCCGTACAGGCAGGGGGCTCACTG |
| hIL27 | | ACCCTGAGCTGTGCAGCTAGTGAGTATGCCTATAGCACTTGCAACATGGGCGGG |
| Ra_VH | | TATCGTCAGGCCCCTGGCAAGGAGCGCGAGCTTGTGAGCGCGTTCATTAGCGAT |
| H7 | | GGCTCCACCTACTATGCAGACAGCGTTAAGGGCCGCTTTACCATCACCCGCGAT |
| | | AACGCTAAAAACACCGTTTACCTCCAGATGAACTCCCTGAAGCCTGAGGATACG |
| | | GCTATCTATTACTGCTCCGCTAATTGTTATCGCAGGCTGCGTAACTACTGGGGT |
| | | CAAGGCACCCAGGTGACCGTGTCTTCCGGCGGTGGCTCCCAGGTCCAGCTGCAA |
| | | GAGTCCGGCGGTGGCCTTGTGCAACCTGGAGGCTCCCTGAGACTTTCCCGCGCT |
| | | GCCTCCGGCTTCACGTTCAGCTCTTATCCCATGAGCTGGGTGAGACAGGGTCCT |
| | | GGTAAGGGCCTGGAATGGATTTCCACCATCTCTGCGGGGGGTGACACGACACTC |
| | | TACGCTGATAGTGTTAAGGGACGTTTCACCTCCTCTCGCGATAACGCGAAGAAC |
| | | ACCCTGTACGTTCAGCTTAATTCCCTGAAAACCGAAGACACCGCTATCTACTAT |
| | | TGCGCCAAGAGAATTGACTGCAACTCCGGTTACTGTTATCGTCGCAACTACTGG |
| | | GGCCAGGGCACCCAGGTGACAGTCTCTTCT |
| DR595- | 1160 | CAGGTCCAGTTGCAGGAGTCTGGGGGGGGCTCAGTGCAAGCCGGGGGCTCCCTG |
| hIL27 | | ACTCTGAGCTGCGCTGCCTCTGAGTATGGTTACTCCACGTGCAACATGGCGGGG |
| Ra_VH | | TATCGCCAGGCCCCCGGAAAGGAACGCGAGCTGGTGTCTGCCTTCATCTCTGAC |
| H7 | | GGCAGCACATATTACGCTGACTCCGTAAAAGGTAGATTTACCATCACCCGCGAC |
| | | AATGCGAAAAACACAGTTTATGTCCAGATGAACTCCCTGAAACCGGAGGACACC |
| | | GCGATCTACTATTGCAGCGCGAATTGTTACCGCCGTCTTCGTAATTACTGGGGG |
| | | CAGGGAACCCAGGTGACAGTCTCCAGGGGGGGGTCCGGTGGCTCCGGTGGGTCC |
| | | GGCCAGGTGCAGCTCCAGGAGTCTGGTGGGGGGTTGGTGCAGCCCGGAGGTAGC |
| | | CTGAGGCTGTCCTGTGCTGCATCTGGTTTTACCTTCAGCAGTTATCCAATGTCC |
| | | TGGGTACGCCAAGCGCCCGGTAAGGGCTTGGAGTGGATTTCTACAATTAGCGCC |
| | | GGAGGCGATACTACCCTGTACGCTGACTCTGTGAAGGGTCGTTTCACATCCTCT |
| | | AGGGATAACGCGAAAAATACACTGTACCTTCAGCTCAATAGTCTGAAGACCGAG |
| | | GACACCGCAATCTATTACTGTGCAAAACGCATCGACTGTAATAGCGGATACTGC |
| | | TACAGGCGTAACTACTGGGGCCAGGGAACCCAGGTGACCGTCAGTAGC |
| DR595- | 1161 | CAGGTACAGTTGCAGGAAAGTGGGGGGGGGTCTGTGCAGGCTGGCGGGTCCTTG |
| hIL27 | | ACCCTGTCATGTGCCGGTTCTGAGTACGCCTACTCCACGTGTAACATGGGCTGG |
| Ra_VH | | TATCGTCAGGCTCCCGGCAAGGAGCGCGAGCTGGTATCCGCCTTCATCTCTGAT |
| H8 | | GGCAGCACCTATTACGCCGATAGCGTGAAGGGTCGTTTCACTATCACACGGGAC |
| | | AACGCGAAGAACACAGTGTATTTGCAAATGAACAGCTTGAAGCCCGAAGATACC |
| | | GCTATCTATTACTGCTCTGCTAACTGTTATCGTCGCCTGCGGAACTACTGGGGG |
| | | CAGGGGACTCAGGTAACTGTTTCCAGCGGAGGCGGTAGCCAGGTCCAGCTTCAG |
| | | GAGAGCGGCGGAGGCTCCGTGCAGGTCGGTGGCTCTCTCCGCCTCTCATGTGCT |
| | | GCCAGCGGATTTACTTTCAGCTCCTACCCCATGTCATGGGTGCGGCAGGGTCCC |
| | | GGAAAGGGCCTTGAATGGATTAGCACGATTAGCGCCGGTGGCGACACAACCCTG |
| | | TACGCCGACTCTGTCAAGGGTCGTTTCACGTCCTCAAGAGACAACGCGAAGAAC |
| | | ACTCTGTACTTGCAGCTCAATTCTCTGAAGACAGAGGACACCGCCATCTATTAC |
| | | TGTGCAAAGAGGATTGACTGCAACAGGGGCTATTGCTACAGACGTAACTATTGG |
| | | GGTCAGGGCACTCAGGTGACTGTGTCCAGT |
| DR595- | 1162 | CAGGTGCAGCTGCAAGAGTCTGGGGGGGGGTCCGTGCAGGCCGGAGGTAGTCTG |
| hIL27 | | ACTCTTTCCTGCGCTGCGTCTGAGTACGCCTACTCCACCTGTAACATGGGATGG |
| Ra_VH | | TACAGACAGGCCCCTGGCAAAGAGAGAGAACTGGTGTCCGCTTTTATCAGCGAC |
| H8 | | GGCAGCACTTACTATGCTGACAGTGTGAAGGGCAGGTTCACCATCACCCGTGAT |
| | | AACGCCAAGAACACTGTCTACGTTCAGATGAACTCTTTGAAACCAGAGGACACA |
| | | GCTATCTACTATTGCAGCGCTAACTGCTACAGACGCCTCAGGAACTATTGGGGG |
| | | CAGGGCACCCAGGTGACCGTTTCTAGTGGAGGTAGCGGAGGCAGCGGTGGAAGC |
| | | GGCCAGGTGCAGCTCCAGGAGTCCGGTGGCGGGTCCGTCCAGGTCGGGGGGTCT |
| | | CTCCGCCTGTCCTGCGCTGCCAGCGGGTTCACCTTCTCCAGCTACCCCATGAGC |
| | | TGGGTCAGGCAGGCCCCTGGCAAGGGCCTCGAATGGATCAGCACTATTTCCGCC |
| | | GGTGGCGACACAACCCTCTACGCCGACAGCGTCAAGGGACGGTTCACCAGCAGT |
| | | CGTGACAACGCTAAAAATACCCTGTACCTCCAGCTTAATAGCCTGAAGACCGAA |
| | | GATACAGCAATCTATTACTGCGCCAAGAGGATCGACTGCAACTCAGGGTACTGT |
| | | TACCGTCGCAACTACTGGGGACAGGGAACTCAAGTGACTGTGTCTTCC |
| DR595- | 1163 | CAGGTCCAGTTGCAGGAGTCCGGCGGTGGAAGTGTGCAGGCTGGTGGCAGCCTG |
| hIL27 | | ACCTTGTCTTGTGCCGCTAGTGAGTACGCTTATTCAACCTGCAACATGGGCTGG |
| Ra_VH | | TACAGACAGGCTCCGGGAAAGGAGCGCGAGCTGGTGTCTGCCTTCATCTCCGAT |
| H9 | | GGTTCAACATACTATGCTGACTCCGTGAAGGGGCGCTTCACCATCACGCGCGAT |
| | | AATGCAAAGAATACCGTGTACCTTCAAATGAATAGTCTGAAACCCGAGGATACC |
| | | GCTATCTATTACTGCTCCGCCAACTGCTATCGTCGCCTGCGTAATTACTGGGGC |
| | | CAGGGCACCCAGGTCACTGTTAGTTCAGGAGGCGGAAGCCAGGTCCAGTTGCAG |
| | | GAGTCCGGGGGTGGCTCCGTCCAGAGCGGCGGTAGCCTGAGATTGAGTTGCGCG |
| | | GCCTCAGGCTTCACCTACAGCACCAGCAATAGCTGGATGGCCTGGTTCCGTCAG |
| | | GCTCCGGGCAAAGAGAGGGAGGGCGTGGCGGCCATCTACACTGTGGGGGGTAGC |
| | | ATCTTCTACGCGGACAGCGTTCGGGGTCGCTTCACTATCAGCCAAGACGCTACC |
| | | AAGAATATGTTCTACCTCCAGATGAACACTCTGAAGCCCGAGGACACCGCCATG |
| | | TACTATTGCGCTGCCGGTTCTGGCCGCGTGCGCGGTAAGTGGTTTTGGCCCTAT |
| | | GAGTACAACTATTGGGGTCAAGGAACCCAGGTGACCGTGTCTTCC |
| DR595- | 1164 | CAGGTCCAGTTGCAGGAGAGCGGTGGGGGATCTGTCCAAGCCGGAGGTAGTCTC |
| hIL27 | | ACGCTCAGCTGTGCCGCGAGCGAATACGCCTACTCCACCTGTAATATGGCGGGG |
| Ra_VH | | TATCGTCAGGCACCAGGCAAAGAGCGCGAACTGGTGTCCGCGTTCATCTCCGAC |
| H9 | | GGCTCAACGTATTACGCCGATAGCGTAAAGGGCCGGTTTACAATCACCCGCGAC |
| | | AACGCCAAGAATACCGTCTACCTTCAGATGAACTCCCTGAAGCCAGAGGACACT |
| | | GCAATTTACTATTGCAGCGCTAACTGTTACCGCCGTCTGCGCAACTACTGGGGA |
| | | CAAGGAACCCAAGTTACCGTGAGTTCTGGTGGCTCTGGTGGCAGCGGAGGCAGG |
| | | GGACAAGTTCAGCTTCAGGAGTCTGGAGGTGGCTCAGTACAGTCTGGTGGCTCC |
| | | CTCCGCCTCAGCTGTGCAGCTTCAGGTTTCACCTACCCCACGTCCAACAGTTGG |
| | | ATGGCCTGGTTTCGGCAAGCTCCGGGAAAAGAGCGTGAGGGAGTGGCTGCGATC |
| | | TATACAGTCGGGGGATCTATCTTCTACGCGGACTCCGTCAGAGGCAGGTTCACC |
| | | ATTTCTCAGGATGCTACTAAAAATATGTTCTATCTTCAGATGAACACGCTGAAG |
| | | CCAGAGGACACAGCCATGTACTATTGCGCCGCAGCCTCAGGCAGGCTGCGCGGT |
| | | AAATGGTTTTGGCCTTACGAGTACAACTATTGGGGTCAGGGCACCCAGGTGACC |
| | | GTGAGTTCT |
| DR595- | 1165 | CAGGTGCAGTTGCAGGAGTCCGGCGGGGGCTCCGTGCAGGGGGGGGGTAGCCTG |
| hIL27 | | ACTCTGTCATGTGCCGCGTCCGAGTACGCTTACTCTACCTGCAACATGGGCTGG |
| Ra_VH | | TATCGCCAGGCTCCTGGAAAAGAACGCGAACTGGTGAGTGCCTTTATCTCTGAC |
| H10 | | GGTTCCACTTATTACGCCGATTCCGTGAAGGGGCGCTTTACAATCACTCGCGAT |
| | | AATGCCAAGAACACAGTGTACCTCCAGATGAACAGCCTGAAACCAGAGGACACC |
| | | GCTATTTACTATTGCAGCGCGAACTGCTACAGACGCCTCAGGAACTATTGGGGA |
| | | CAGGGCACCCAGGTGACAGTTTCCAGTGGAGGTGGCTCCAGGTCCCAGTTGCAG |
| | | GAAAGTGGGGGGGGTCCGTCCAAGCTGGCGGTTCTCTGCGCGCTCTCATGCAGG |
| | | GCCTCCGGTTCCACTTATAGTAACTATTGCTTGGGCTGGTTCAGGCAGATCACC |
| | | GGCAAGGAGCGCGAGGGAGTGGCTGTCATCAACTGCGGGGGCGGTATGGTTTAT |
| | | TTCGCCGACTCCGTGAAGGGCCGTTTTACAGTGTCACAAGACCAGGCCAAAAAT |
| | | ACTCTGTACCTGCAAATGAACAGTTTGAAGCCTGAGGATACCGCGATGTACTAT |
| | | TGTGCCGCTGAGTCCGTAAGTAGCTTTAGCTGCGGCGGTTGGTTGACCAGACCC |
| | | GACCGCGTGCCATATTGGGGTCAGGGCACACAGGTGACAGTGAGCAGC |
| DR595- | 1166 | CAAGTGCAGTTGCAGGAAAGCGGAGGCGGGTCTGTTCAGGCCGGAGGCAGCCTT |
| hIL27 | | ACGCTGTCCTGCGCTGCCTCAGAATACGCATACAGTACCTGTAACATGGGCTGG |
| Ra_VH | | TATCGCCAGGCCCCTGGGAAGGAGAGAGAGCTGGTGAGTGCCTTCATTTCTGAC |
| H10 | | GGCTCTACCTACTATGCTGATAGCGTGAAGGGTAGGTTTACAATCACCCGCGAC |
| | | AACGCCAAAAATACCGTTTACCTTCAGATGAACAGCCTGAAGCCGGAAGACACC |
| | | GCCATCTATTACTGCTCTGCCAACTGCTATCGCCGTCTGCGCAATTACTGGGGG |
| | | CAGGGCACCCAGGTCACCGTGTCCAGCGGCGGTTCTGGTGGGTCTGGAGGGTCC |
| | | GGGCAGGTGCAGCTGCAAGAGTCTGGAGGTGGGTCCGTCCAGGCCGGGGGCAGT |
| | | CTGCGCTTGTCATGTCGGGCCTCTGGTTCCACTTACTCCAACTACTGCTTGGGA |
| | | TGGTTCCGGCAGATTACAGGCAAGGAACGCGAGGGAGTCGCAGTCATCAACTGG |
| | | GTGGGCGGAATGCTTTACTTTGCCGATTCCGTCAAGGGCAGGTTCACTGTGAGC |
| | | CAGGATCAGGCTAAGAACACACTGTACCTTCAGATGAATAGCCTGAAGCCCGAG |
| | | GACACTGCTATGTATTACTGTGCTGCCGAAAGCGTGTCCTCATTCTCCTGCGGA |
| | | GGCTGGCTGACCCGCCCTGATCGGGTGCCGTACTGGGGTCAAGGCACTCAAGTG |
| | | ACTGTCTCATCT |
| DR595- | 1167 | CAAGTACAGTTGCAGGAGAGCGGTGGCGGTTCAGTGCAGGCCGGGGGAAGTCTG |
| hIL27 | | ACCCTGAGTTGCGCGGCCAGCGAGTATGCCTACAGTACCTGTAACATGGGCTGG |
| Ra_VH | | TATCGCCAAGCACCTGGCAAGGAACGTGAGCTGGTTTCAGCCTTCATCTCCGAC |
| H11 | | GGCTCCACCTATTACGCTGACAGCGTGAAGGGTAGGTTCACTATTACTCGGGAC |
| | | AACGCGAAGAATACCGTATACTTGCAGATGAACTCACTGAAGCCTGAGGACACC |
| | | GCCATCTATTACTGCTCCGCCAACTGCTACCGTCGCCTTCGTAATTATTGGGGG |
| | | CAGGGAACCCAGGTCACTGTCAGCTCTGGAGGCGGTTCACAAGTACAGTTGCAG |
| | | GAGTCTGGTGGGGGTAGCGTCCAGGGGGGGGGAAGCCTTCGCCTTTCTTGCCGC |
| | | GCCAGCGGCTCCACCTACTCAAATTACTGCCTCGGATGGTTTCGCCAGTCTACC |
| | | GGAAAGGAACGCGAGGGTGTCGCAGTGATAAATTGGGTCGGGGGTATGCTGTAG |
| | | TTCGCGGATTCTGTGAAGGGCAGGTTCACAGTTTCCCAGGATCACGCCAAGAAG |
| | | ACCGTGACCGTCCAGATGAACTCCCTGAAGCCGGAAGATACCGCAATGTATTAC |
| | | TGTGCCGCTGAGTCTGTGTCATCTTTTTCTTGCGGTGGATGGTTGACTCGCCCC |
| | | GGTCGCGTGCCTTATTGGGGCCAAGGCACACAAGTGACAGTGTCTAGC |
| DR595- | 1168 | CAGGTGCAGTTGCAGGAAAGCGGTGGAGGGTCCGTGCAGGCCGGGGGCTCTCTG |
| hIL27 | | ACACTGTCTTGTGCCGCTAGTGAGTACGCATACAGCACATGCAACATGGGCTGG |
| Ra_VH | | TATCGTCAAGCTCCGGGTAAGGAGCGTGAGCTTGTCTCCGCATTCATCAGTGAC |
| H11 | | GGCTCAACCTATTACGCGGACAGCGTGAAGGGAAGATTTACCATCACCCGCGAT |
| | | AACGCCAAGAACACTGTCTACCTCCAGATGAACAGCCTGAAACCTGAGGATACT |
| | | GCTATTTACTATTGCAGTGCAAACTGTTACCGTCGCCTTCGCAACTATTGGGGC |
| | | CAGGGCACACAAGTGACTGTGAGTTCAGGTGGCTCCGGTGGCTCAGGTGGGTCC |
| | | GGTCAGGTCCAGCTCCAGGAGTCCGGCGGAGGCTCCGTGCAGGCAGGGGGCTCT |
| | | CTGAGGCTGTCCTGTAGGGCATCCGGTTCCACCTATAGTAACTACTGCCTGGGC |
| | | TGGTTCCGCCAGAGCACCGGAAAGGAACGCGAGGGCGTGGCCGTCATCAACTGG |
| | | GTCGGGGGCATGTTGTACTTCGCGGACTCTGTGAAGGGCCGCTTCACCGTGTCC |
| | | CAAGATCACGCCAAAAACACTGTGACTCTCCAGATGAATAGCCTCAAGCCCGAA |
| | | GATACGGCGATGTACTATTGCGCAGCCGAGTCCGTGAGTTCCTTCTCATGCGGT |
| | | GGCTGGCTGACCCGCCCCGGTCGCGTGCCTTACTGGGGCCAGGGTACTCAGGTG |
| | | ACCGTCTCCTCC |
| DR595- | 1169 | CAGGTGCAGTTGCAGGAGTCCGGGGCGGGGAGCGTACAGGCTGGCGGTTCTCTG |
| hIL27 | | ACACTTTCCTGCGCAGCCAGCGAGTACGCCTACTCCACCTGCAACATGCGGGGG |
| Ra_VH | | TATCGCCAAGCGCCTGGAAAGGAGCGCGAGCTGGTTAGCGCCTTCATCAGTGAT |
| H12 | | GGCTCCACATATTACGCGGACAGCGTGAAGGGACGCTTCACCATCACCCGTGAC |
| | | AACGCGAAAAACACAGTCTACTTGCAGATGAACTCCCTCAAGCCTGAGGATACA |
| | | GCCATCTACTATTGCTCCGCCAACTGCTATAGGAGATTGCGCAACTACGGGGGG |
| | | CAAGGAACGCAGGTCACCGTCTCCTCAGGGGGAGGCAGCCAAGTGCAACTCCAG |
| | | GAGAGCGGGGGTGGCAGCGTGCAGGCGGGTGAGTCTCTCAGACTGTCTTGCCGC |
| | | GCGTCTGGCTCTACATATTCTAACTATTGCCTGGGTTGGTTCCGGCAGATTACT |
| | | GGCAAGGAGCGCGAGGGTGTTGCTGTGATTAACTGGGGGCGGGGTATGCTGTAC |
| | | TTCGCCGATTCCGTCAAGGGACGTTTCACCGTTAGCCAGGACCAGGCCAAAAAC |
| | | ACCGTCTACCTGGAGATGAATAGCTTGAAGCCCGAAGATACCGCGATGTATTAC |
| | | TGCGCCACCGAGAGTGTCTCATCTTTTTCCCGTGGGGGTTGGGTTACTCGCCCA |
| | | GACAGGGGCCATACTGGGGACAGGGCACTCAGGTGACTGTAAGCAGC |
| DR595- | 1170 | CAGGTTCAGTTGCAGGAGAGTGGCGGAGGCAGCGTTCAGGCCGGAGGCTCTCTG |
| hIL27 | | ACCCTGTCATGCGCGGCCAGCGAGTACGCCTACAGCACCTGCAACATGGGATGG |
| Ra_VH | | TATCGCCAGGCACCCGGTAAGGAGAGAGAACTGGTGTCCGCCTTCATCTCTGAC |
| H12 | | GGCTCCACTTACTATGCTGATAGCGTGAAGGGTCGCTTTACCATCACGCGCGAT |
| | | AACGCAAAAAATACCGTGTATTTGCAGATGAACTCTCTCAAGCCCGAGGATACC |
| | | GCCATTTATTACTGTAGCGCTAACTGTTACCGTCGCCTGCGCAACTATTGGGGC |
| | | CAGGGCACCCAGGTTACTGTCAGCTCCGGTGGCTCCGGCGGTTCTGGGGGCTCC |
| | | GGCCAGGTGCAGCTCCAGGAGTCTGGTGGAGGCTCCGTGCAGGCAGGTGAGTCC |
| | | TTGCGTCTGTCCTGCCGGGCCAGCGGTTCAACATATTCTAACTACTGCCTGGGG |
| | | TGGTTCCGCCAGATTACCGGCAAAGAGCGCGAGGGTGTCGCAGTCATCAACTGG |
| | | GTCGGAGGTATGCTGTACTTCGCCGATTCTGTGAAGGGACGTTTCACCGTGTCC |
| | | CAGGACCAGGCCAAGAACACAGTATATGTGGAGATGAACTCCCTGAAGCCTGAA |
| | | GATACAGCCATGTATTACTGCGCCACGGAGTCCGTCAGCTCCTTCTCCTGTGGC |
| | | GGATGGTTGACCAGGCCCGACAGGGTGCCTTATTGGGGTCAGGGCACCCAAGTC |
| | | ACCGTGAGCAGC |
| DR595- | 1171 | CAGGTGCAGGTCCAGGAGTCCGGCGGTGGCTCTGTGCAGGGGGGGGGTAGCCTT |
| hIL27 | | ACTCTCAGCTGTGCTGCGTCTGAATACGCTTATTCTACCTGTAATATGGGCTGG |
| Ra_VH | | TATCGGCAGGCCCCTGGCAAGGAGAGAGAACTCGTGAGCGCCTTCATCTCCGAC |
| H13 | | GGCTCTACCTACTATGCCGACAGTGTGAAGGGCCGCTTCACTATTACTCGCGAT |
| | | AACGCTAAGAACACGGTGTACCTCCAGATGAACTCTCTGAAGCCGGAGGACACG |
| | | GCCATTTATTACTGCTCCGCTAACTGCTACAGACGGCTGCGTAACTACTGGGGG |
| | | CAGGGCACCCAGGTAACCGTCAGCTCCGGCGGAGGTTCTCAGGTTCAGCTCCAA |
| | | GAGTCTGGGGGGGGGAGCGTCCAGGCCGGAGGGTCCCTCAGGTTGTCCTGCGTG |
| | | GCCAGCGGATATGTGTCTTGTGACTACTTCCTGCCCAGCTGGTATCGCCAGGCT |
| | | CCAGGGAAGGAACGTGAGTTTGTTTCCATCATTGATGGTACAGGCAGCACATCC |
| | | TACGCCGCATCTGTGAAGGGCCGGTTCACTGCCAGCCAGGACCGTGGCAAAAAC |
| | | ATCGCTTACTTGCAGATGAACTCCCTGAAGCCCGAGGATACCGCAATGTACTAT |
| | | TGCAAGGCGAGCTGTGTGCGTGGCAGAACCATTAGCGAATACTGGGGACAGGGC |
| | | ACGCAGGTGACCGTCTCCTCC |
| DR595- | 1172 | CAGGTCCAACTCCAGGAGTCTGGCGGAGGTTCCGTTCAGGCCGGTGGGTCCCTT |
| hIL27 | | ACCCTGAGTTGCGCCGCTTCAGAATACGCCTACTCAACCTGTAACATGGGCTGG |
| Ra_VH | | TATCGCCAAGCTCCTGGTAAGGAAAGAGAGTTGGTGTCCGCTTTCATCTCTGAT |
| H13 | | GGTTCTACCTATTACGCGGACTCTGTGAAGGGCAGATTTACCATCACAAGAGAC |
| | | AACGCCAAGAATACCGTGTACCTCCAGATGAACTCTCTGAAGCCTGAGGATACT |
| | | GCCATCTATTACTGTTCAGCAAACTGCTACCGTCGCCTGCGCAATTACTGGGGG |
| | | CAGGGCACCCAGGTGACAGTATCCAGCGGTGGCAGTGGAGGCTCCGGTGGCTCT |
| | | GGCCAAGTGCAGCTCCAGGAGTCCGGGGGGGGTTCAGTGCAGGCCGGGGGCTCC |
| | | CTGCGGCTGTCCTGTGTCGCGTCTGGATACGTGAGTTGCGACTACTTTCTCCCC |
| | | TCCTGGTATCGCCAGGCTCCAGGAAAGGAGCGCGAGTTTGTCAGCATCATTGAT |
| | | GGCACCGGGAGTACCAGTTATGCGGCTTCTGTTAAGGGCCGTTTCACCGCTTCT |
| | | CAGGATCGCGGAAAGAACATCGCCTATTTGCAGATGAACTCTCTGAAGCCGGAA |
| | | GACACAGCCATGTATTACTGCAAAGCCAGTTGTGTCAGGGGACGGACCATCTCC |
| | | GAGTATTGGGGACAGGGTACGCAGGTCACTGTGAGTTCC |
| DR595- | 1173 | CAGGTGCAGCTTCAGGAGTCTGGAGGCGGATCTGTCCAGGCCGGGGGCAGTCTG |
| hIL27 | | ACCCTGTCCTGCGCCGCTTCTGAGTATGCCTATAGTACTTGCAACATGGGTTGG |
| Ra_VH | | TATCGGCAGGCTCCGGGCAAGGAGCGCGAACTGGTTTCAGCGTTCATCTCTGAC |
| H14 | | GGTAGCACCTACTATGCCGACAGCGTGAAGGGCCGCTTTACTATCACCAGGGAT |
| | | AACGCAAAGAACACCGTTTACCTCCAGATGAATAGCCTCAAGCCTGAGGATACC |
| | | GCTATCTATTACTGCTCAGCTAACTGTTATCGTCGGCTGCGTAACTACTGGGGC |
| | | CAGGGAACTCAAGTTACCGTCAGCAGTGGGGGAGGCAGTCAGGTGCAGTTGCAG |
| | | GAAAGCGGGGGGGGCAGCGTCCAGGCTGGTGGAAGCCTTCGTCTGTCTTGTGTC |
| | | GCCAGTGGCTATGTCTCCTGTGATTATTTTCTGCCGTCCTGGTATCGTCAAGCG |
| | | CCTGGCAAGGAAAGGGAGTTCGTATCCATTATCGACGGAACTGGTAGTACCAGC |
| | | TACGGTGCCTCCGTGAAGGGAAGGTTCACAGCCTCCCAGGATAAGGGTAAGAAC |
| | | ATTGCCTACCTCCAGATGAACTCCCTGAAGCCTGAAGATACGGCCATGTACTAT |
| | | TGCAAAGCCTCTTGTGTCAGGGGCAGAGCCATCAGCGAATACTGGGGCCAAGGG |
| | | ACTCAGGTCACCGTGAGCAGG |
| DR595- | 1174 | CAGGTTCAGGTCCAGGAGTCTGGGGGGGGTTCTGTCCAGGCCGGAGGCAGCCTT |
| hIL27 | | ACACTGTCTTGCGCTGCCAGCGAGTATGCTTACTCTACCTGCAACATGGGTTGG |
| Ra_VH | | TATCGGCAAGCGCCGGGCAAGGAACGGGAACTCGTCTCTGCTTTCATCCCCGAT |
| H14 | | GGCAGCACATATTACGCTGATAGCGTAAAGGGCCGGTTCACCATCACAAGAGAC |
| | | AACGCAAAGAACACAGTTTACCTCCAGATGAACTCCCTCAAACCTGAGGATACC |
| | | GCCATCTATTACTGCTCCGCTAACTGCTACCGCCGTCTGCGCAACTACTGGGGT |
| | | CAAGGCACCCAGGTTACCGTGTCTAGCGGGGGGTCTGGTGGCAGTGGTGGGTCA |
| | | GGGCAGGTCCAGCTCCAGGAGAGCGGGGGAGGTAGCGTACAGGCCGGGGGTTCA |
| | | CTGAGACTGTCTTGTGTGGCCTCTGGATACGTCTCTTGCGATTACTTCCTTCCC |
| | | AGTTGGTATAGGCAAGCGCCCGGTAAGGAGAGAGAGTTCGTTAGCATCATTGAT |
| | | GGCACAGGTTCAACAAGTTATGCGGCTTCCGTAAAGGGACGCTTTACTGCGTCC |
| | | CAGGACAAGGGCAAAAACATCGCCTATCTTCAAATGAACAGTCTGAAGCCTGAA |
| | | GATACCGCTATGTATTACTGCAAAGCCTCCTGGGTGCGCGGTCGCGCTATCAGC |
| | | GAATACTGGGGCCAGGGTACGCAAGTGACTGTCAGTTCT |
| DR595- | 1175 | CAGGTTCAACTCCAGGAATCAGGAGGCGGTTCAGTGCAGGCTGGGGGTAGCCTG |
| hIL27 | | ACCCTCAGCTGCGCTGCGAGTGAGTATGGTTATTCCACCTGCAACATGGCGGGG |
| Ra_VH | | TATCGCCAAGCGCCTGGCAAGGAGAGAGAGCTGGTCAGTGCATTTATTTCCGAT |
| H15 | | GGCTCCACCTACTATGCTGACTCAGTGAAGGGGCGCTTCACCATCACTCGCGAC |
| | | AACGCCAAGAATACTGTCTATCTCCAGATGAACAGCCTCAAACCAGAGGATACA |
| | | GCCATCTACTATTGCAGTGCGAATTGTTATCGTAGGCTGCGCAACTACTGGGGC |
| | | CAAGGCACACAGGTGACCGTGTCTAGTGGAGGTGGCTCTCAGGTCCAGCTCCAG |
| | | GAATCTGGTGGAGGCTCCGTCCAGGCTGGCGGTTCCCTTCGTCTGTCCCGCGTG |
| | | GCCAGCGGCTACGTGAGCTGTGATTATTTCCTCCCTAGGTGGTATCGGCAGGCC |
| | | CCCGGCAAGGAACGCGAGTTCGTTTCTATCATTGATGGAACGGGTTCTACTAGC |
| | | TACGCGGCCTCCGTGAAGGGGCGCTTCACAGCAAGCCAGGACAAAGGGAAGAAC |
| | | ATCGCTTACCTGCAAATGAATACCTTGAAGCCTGAGGACACCGCAATGTATTAC |
| | | TGCAAGGCTTCCTGCGTGAGGGGCCGCGCCATGTCCGAATACTGGGGGCAGGGT |
| | | ACTCAGGTGACCGTTAGCAGT |
| DR595- | 1176 | CAGGTCCAGCTCCAGGAGTCCGGGGGGGGATCAGTCCAGGCTGGGGGGAGTCTG |
| hIL27 | | ACACTGTCCTGTGCTGCCTCTGAGTATGCTTACAGCACCTGTAACATGGGCTGG |
| Ra_VH | | TACAGACAAGCCCCTGGGAAGGAGCGCGAACTCGTGTCCGCGTTTATTAGTGAC |
| H15 | | GGTTCCACCTACTATGCTGACTCCGTGAAGGGTCGCTTCACGATCACCCGCGAC |
| | | AACGCCAAAAATACCGTGTACTTGCAGATGAACTCCCTGAAGCCGGAGGACACA |
| | | GCCATCTATTACTGTAGCGCAAATTGCTACCGTCGCCTGCGTAACTATTGGGGT |
| | | CAGGGCACTCAGGTCACAGTCAGTAGCGGCGGTAGCGGTGGCTCCGGCGGTAGT |
| | | GGACAGGTGCAGTTGCAGGAATCCGGGGGTGGAAGTGTTCAAGCAGGGGGCTCT |
| | | CTGCGCCTGTCATGTGTTGCATCAGGCTATGTCTCTTGCGACTACTTCCTGCCA |
| | | TCTTGGTATCGTCAGGCCCCAGGGAAGGAAAGGGAGTTCGTCTCCATCATTGAT |
| | | GGCACCGGCTCAACCTCTTACGCTGCCAGCGTAAAGGGGCGCTTCACCGCCTCC |
| | | CAGGATAAGGGCAAGAACATCGCTTACCTCCAGATGAACACACTCAAGCCTGAG |
| | | GATACCGCGATGTATTACTGTAAGGCGAGTTGCGTTCGTGGCCGTGCGATCAGC |
| | | GAGTATTGGGGGCAGGGAACCCAGGTAACAGTCAGCTCC |
| DR595- | 1177 | CAAGTGCAGGTCCAGGAGTCCGGGGGGGGTAGCGTGCAGGCCGGTGGCTCCCTC |
| hIL27 | | ACTCTGTCCTGCGCTGCCAGCGAGTATGCGTACTCCACCTGCAACATGGGATGG |
| Ra_VH | | TACAGGCAAGCGCCTGGCAAGGAAAGAGAGCTTGTGAGCGCCTTCATCTCCGAT |
| H16 | | GGTTCTACCTACTATGCTGACTCCGTTAAGGGCCGGTTCACCATTACTCGCGAT |
| | | AACGCCAAGAACACCGTGTATCTCCAGATGAACTCCCTCAAGCCAGAGGACACA |
| | | GCTATCTACTATTGCTCAGCTAACTGTTACCGTCGCCTCCGCAACTATTGGGGC |
| | | CAGGGCACTCAGGTGACTGTCTCTTCGGGGGCGGGCTCCCAAGTGCAGCTCCAG |
| | | GAGTCAGGAGGCGGGTCTGTCCAGGGGGGTGGCTCCCTGCGCCTCAGCTGCCGT |
| | | GCGTGTGGCAGCACCTACAGCAACTACTGTTTGGGCTGGTTCAGACAGATTACC |
| | | GGAAAAGAACGCGAGGGCGTTGCGGTCATCAACTGGGTGGGGGGGATGCTTTAC |
| | | TTTGCAGACAGTGTGAAGGGTCGCTTTACCGTCAGCCAGGACCAGGCCAAGAAC |
| | | ACAGTGTATTTGCAGATGAATAGCCTGAAGCCAGAGGACACTGCCATGTATTAC |
| | | TGCGCGGCTGAGTCAGCCAGCTCTTTCAGTTGCGGTGGCTGGTTGACGCGCCCT |
| | | GACCGCGTTCCTTACTGGGGGCAGGGGACTCAGGTTACCGTGAGCAGC |
| DR595- | 1178 | CAGGTGCAGCTTCAGGAATCAGGGGGTGGAAGCGTGCAGGCAGGAGGCTGTCTG |
| hIL27 | | ACTTTGTCATGTGCCGCTAGTGAGTATGCGTATTCTACGTGCAACATGGGCTGG |
| Ra_VH | | TATCGTCAAGCCCCCGGAAAAGAGCGTGAACTCGTGTCAGCTTTCATTTCCGAC |
| H16 | | GGTAGCACATATTACGCCGACAGCGTGAAGGGGCGCTTTACTATCACTCGCGAT |
| | | AATGCCAAGAATACAGTTTACCTCCAGATGAACTCACTGAAGCCCGAAGACACC |
| | | GCCATCTACTATTGTAGCGCCAACTGTTATAGGAGACTGCGGAACTACTGGGGT |
| | | CAAGGCACCCAGGTGACCGTGTCCTCTGGAGGCTCTGGAGGCTCCGGGGGTAGC |
| | | GGACAGGTGCAGTTGCAGGAGTCCGGTGGGGGGTCAGTGCAGGCCGGGGGCTCA |
| | | CTCAGGCTTTCCTGCCGCGCCAGTGGTTCCACTTATTCCAACTATTGCCTGGGC |
| | | TGGTTCAGGCAGATCACAGGTAAGGAACGTGAGGGGGTGGCTGTGATTAACTGG |
| | | GTCGGCGGGATGCTGTATTTTGCCGACAGTGTGAAAGGGAGATTCACCGTCAGC |
| | | CAGGATCAGGCCAAAAACACCGTGTACCTGCAAATGAACAGTCTGAAGCCAGAG |
| | | GATACCGCCATGTATTACTGTGCAGCGGAGAGCGCCTCCAGCTTTTCCCGCGGA |
| | | GGTTGGCTCACTCGGCCTGACCGCGTTCCCTACTGGGGTCAAGGGACCCAGGTG |
| | | ACAGTCTCCTCC |
| DR595- | 1179 | CAGGTCCAACTCCAAGAAAGCGGTGGGGGTTCCGTCCAGGCCGGAGGCTCCCTG |
| hIL27 | | ACCCTGTCCTGTGCTGCAAGTGAGTACGCCTATTCCACCTGTAATATGGGTTGG |
| Ra_VH | | TATAGGCAGGCACCAGGAAAAGAGCGCGAACTGGTGTCCGCGTTCATCTCCGAC |
| H17 | | GGCTCCACTTACTATGCTGATAGTGTTAAAGGCCGCTTCACCATCACCCGTGAT |
| | | AACGCGAAGAACACTGTGTACTTGCAGATGAACAGCCTGAAGCCCGAGGATACA |
| | | GCCATCTACTATTGCTCTGCCAACTGCTATAGAAGGCTGCGGAACTACTGGGGT |
| | | CAGGGAACCCAGGTGACCGTGAGTTCCGGGGGGGGTTCCCAGGTCCAGCTCCAG |
| | | GAATCTGGCGGGGGTCTGGTGCAGCCTGGAGGTTCACTCAGATTGTCTTGCGCC |
| | | GCGTCTGGCTTCACATTCTCCGTCTCCGGGATGAGTTGGGTCCGCCAAGCCCCC |
| | | GGCAAGGGTCTGGAGTGGGTATCCGCCATCAGTTCCGGCGGAGCCTCAACCTAC |
| | | TATACGGACTCCGTGAAGGGACGTTTTACCATCAGCCGGGACAACGCTAAGAAC |
| | | ATGCTGTACCTCCAGCTGAACTCTCTGAAGACTGAAGACACCGCCATGTATTAC |
| | | TGTGCCAAGGGTGGCTCCGGGTACGGGGATGCCAGCCGCATGACCTCTCCTGGT |
| | | AGCCAGGGCACGCAGGTCACAGTGTCTTCT |
| DR595- | 1180 | CAGGTACAGCTTCAGGAATCTGGAGGCGGAAGCGTGCAGGCTGGTGGCTCTCTG |
| hIL27 | | ACACTGTCTTGCGCCGCGAGCGAATACGCATACAGTACGTGTAATATGGGTTGG |
| Ra_VH | | TACAGGCAAGCTCCGGGCAAGGAAAGAGAACTCGTAAGCGCCTTCATCAGCGAT |
| H17 | | GGCTCCACTTATTACGCAGACAGCGTGAAGGGCCGCTTCACTATCACGAGGGAT |
| | | AACGCCAAGAACACTGTCTATCTCCAGATGAACTCCCTCAAACCTGAAGACACC |
| | | GCCATTTATTACTGCTCCGCTAATTGTTACCGCCGTCTGCGCAATTATTGGGGA |
| | | CAGGGCACCCAGGTAACAGTGTCCTCTGGAGGGAGCGGTGGATCTGGAGGCTCC |
| | | GGCCAAGTCCAGTTGCAGGAAAGCGGAGGCGGGTTGGTGCAGCCTGGTGGCAGC |
| | | CTGCGCCTGTCTTGCGCGGCCTCTGGCTTCACATTCTCCCTGTCTGGAATGTCT |
| | | TGGGTCCGGCAGGCCCCAGGTAAGGGCCTGGAGTGGGTGAGCGCAATTTCCTCT |
| | | GGCGGGGCATCCACCTATTACACCGACTCCGTGAAGGGCAGGTTCACTATTAGC |
| | | CGGGATAATGCGAAGAACATGCTGTACCTTCAGCTGAACTCCTTGAAGACTGAG |
| | | GACACCGCTATGTATTACTGTGCCAAGGGGGGTAGCGGGTACGGTGACGGTTCC |
| | | CGCATGACCTCTCCGGGTTCCCAGGGCACCCAGGTGACCGTGTCTTCC |
| DR595- | 1181 | CAGGTCCAGTTGCAGGAATCCGGGGGGGGTTCCGTCCAAGCAGGCGGTTCCCTT |
| hIL27 | | ACACTCAGCTGCGCTGCCAGCGAGTACGCCTATTCTACGTGTAACATGGGCTGG |
| Ra_VH | | TATCGCCAGGCTCCTGGGAAGGAACGCGAGCTGGTGAGCGCATTTATCTCAGAT |
| H18 | | GGGTCCACATACTATGCTGACTCTGTTAAGGGTCGTTTCACTATCACCAGGGAC |
| | | AACGCTAAGAACACTGTCTATCTGCAAATGAACTCCCTGAAACCAGAGGACACC |
| | | GCTATCTATTACTGCTCTGCCAACTGTTATCGTCGGTTGCGCAATTATTGGGGG |
| | | CAGGGGACACAGGTGACCGTGTCCTCAGGTGGAGGCTCCCAGGTGCAGTTGCAG |
| | | GAATCTGGGGGAGGCTCCGTGCAGGCTGGCGGTTCACTGAGGCTGAGTTGTGTT |
| | | GCATCTGGCTATGTAAGTTGTGATTACTTCTTGCCAAGCTGGTATCGTCAGGCC |
| | | CCAGGGAAGGAACGCGAGTTCGTCTCTATTATCGACGGCACGGGTTCTACCAGC |
| | | TACGCTGCCAGCGTTAAGGGGCGGTTTACGGCATCCCAGGACAAGGGAAAGAAC |
| | | ATCGGTTATTTGCAGATGAACTCCCTGAAACCCGAGGATACTGCGATGTATTAC |
| | | TGCAAGGCCAGCTGTGTAAGGGGGCGCGGCATCAGCGAATATTGGGGCCAGGGA |
| | | ACTCAGGTGACCGTGTCCTCT |
| DR595- | 1182 | CAGGTGCAGCTCCAGGAAAGCGGCGGGGGCTCCGTGCAGGCTGGGGGCAGCTTG |
| hIL27 | | ACACTGTCCTGTGCAGCTTCCGAGTACGCCTACTCAACCTGCAACATGGGCTGG |
| Ra_VH | | TACAGGCAGGCTCCCGGTAAGGAGCGCGAACTGGTGTCCGCCTTCATTTCTGAC |
| H18 | | GGCTCCACCTATTACGCCGACTCAGTGAAAGGTAGGTTCACCATTACAAGGGAC |
| | | AACGCCAAGAACACTGTCTACTTGCAGATGAACAGTTTGAAGCCCGAAGACACT |
| | | GCTATCTACTATTGCAGCGCCAACTGTTACAGGCGTCTGCGCAACTATTGGGGT |
| | | CAGGGCACCCAAGTGACTGTGTCTTCCGGGGGTCCGGTGGCTCCGGGCGGCTCC |
| | | GGCCAGGTGCAGCTGCAAGAGTCCGGCGGTGGGTCCGTACAGGCGGGGGGCAGC |
| | | CTCAGACTGTCTTGCGTAGCCTCCGGCTACGTTTCTTGCGACTACTTTCTTCCT |
| | | TCTTGGTACAGACAAGCTCCAGGGAAGGAACGGGAGTTCGTGAGCATTATCGAC |
| | | GGCACCGGGTCAACCTCTTACGCGGCCTCCGTGAAGGGCCGTTTTACCGCCAGC |
| | | CAAGACAAGGGCAAAAACATCGCGTATCTTCAGATGAACTCCCTTAAACCTGAG |
| | | GACACCGCGATGTACTATTGCAAGGCTTCTTGCGTTCGCGGGGGGGGGATCAGC |
| | | GAGTATTGGGGCCAGGGCACCCAGGTGACCGTGTCTTCT |
| DR595- | 1183 | CAGGTGCAGTTGCAGGAGTCTGGCGGGGGCTCCGTTCAAGCGGGGGGATCTCTC |
| hIL27 | | ACCCTGTCATGTGCCGCTTCTGAGTATGCCTATTCCACTTGCAACATGGGCGGG |
| Ra_VH | | TATCGCCAGGCACCGGGCAAGGAACGCGAACTCGTGTCTGCCTTCATCCCTGAT |
| H19 | | GGCTCCACCTATTACGCAGATTCCGTCAAGGGCAGATTTACCATTACCAGAGAC |
| | | AACGCTAAGAACACCGTTTACCTCCAGATGAACTCCCTGAAACCTGAGGATACA |
| | | GCCATTTATTACTGTTCCGCCAACTGCTATAGACGCCTGCGGAACTATTGGGGA |
| | | CAAGGCACGCAGGTTACTGTGTCCTCTGGGGGGGGATCTCAGGTGCAGCTTCAG |
| | | GAGAGCGGTGGCGGGTCTGTCCAGGCTGGAGGGAGCTTGAGACTGTCCTGTCGT |
| | | GCGTCCGGGTCAACCTATTCCAACTATTGCCTGGGTTGGTTTCGCCAGATCACT |
| | | GGTAAGGAGCGTGAGGGGGTCGCTGTTATCAACTGCGGGGCGGGGATGCTGTAT |
| | | TTCGCTGATTCAGTAAAGGGCCGCTTCACCGTCTCCCAGGACCAGGCGAAGAAC |
| | | ACCGTGTATCTTCAGATGAACAGCCTCAAGCCTGAGGACACCGCAATGTATTAC |
| | | TGCGCAGCTGAGTCTGTTTCCAGCTTCAGTTGGGGTGGATGGGTTACACGCCCA |
| | | GATCGCGTGCCATACTGGGGTCAGGGTACGCAAGTTACCGTCTCCTCC |
| DR595- | 1184 | CAGGTGCAGGTCCAGGAGTCCGGTGGCGGTTCCGTCCAGGCTGGTGGCTCTCTG |
| hIL27 | | ACGTTGAGTTGCGCGGCCAGCGAGTACGCTTACTCAACGTGCAACATGGCGGGG |
| Ra_VH | | TATCGCCAAGCGCCAGGGAAAGAGCGCGAACTCGTAAGTGCCTTTATTTCCGAT |
| H19 | | GGAAGCACCTATTACGCAGACAGTGTGAAGGGGCGCTTTACTATTACCCGTGAC |
| | | AACGCCAAAAACACCGTCTACCTGCAAATGAACTCCCTGAAACCGGAGGACACC |
| | | GCCATTTATTACTGTTCCGCCAACTGCTATCGCCGTCTGCGCAATTATTGGGGC |
| | | CAGGGGACCCAGGTGACAGTGAGTTCTGGGGGGAGCGGGGGGAGCGGAGGGAGC |
| | | GGGCAGGTCCAGCTTCAGGAGAGCGGGGGAGGCAGCGTGCAGGGTGGGGGTAGC |
| | | CTGCGCTTGTCTTGCAGAGCCTCCGGTTCCACATACAGTAACTATTGCCTGGGA |
| | | TGGTTCCGCCAGATTACCGGCAAGGAACGGGAAGGAGTCGCGGTCATCAACTGG |
| | | GTCGGTGGAATGCTGTATTTTGCTGACTCTGTCAAAGGCCGCTTTACAGTGTCC |
| | | CAGGATCAGGCTAAGAATACTGTCTATCTCCAGATGAACAGCCTGAAGCCTGAA |
| | | GACACAGCCATGTATTACTGTGCCGCAGAGTCCGTCAGCTCCTTGTCCTGCGGT |
| | | GGGTGGCTTACCAGGCCTGATCGCGTTCCTTACTGGGGTCAGGGTACACAGGTG |
| | | ACGGTGTCCAGT |
| DR595- | 1185 | CAGGTACAGCTCCAGGAGAGCGGCGGAGGTTCCGTGCAGGCCGGTGGCAGTCTC |
| hIL27 | | ACCCTGTCCTGCGCTGCCTCTGAGTATGCGTATTCTACCTGTAATATGGGCTGG |
| Ra_VH | | TACAGGCAGGCTCCAGGCAAAGAGAGAGAACTCGTGTCTGCTTTCATCTCTGAC |
| H20 | | GGTAGTACCTATTACGCGGACAGCGTTAAAGGCAGGTTCACCATCACCAGAGAC |
| | | AACGCCAAAAACACAGTTTACCTCCAGATGAACTCCCTCAAGCCCGAGGACACC |
| | | GCAATTTACTATTGTTCCGCTAACTGCTATCGCAGACTGCGTAACTATTGGGGT |
| | | CAGGGTACTCAGGTTACCGTGTCTAGCGGCGGGGGCAGTCAGGTTCAGCTTCAG |
| | | GAAAGTGGCGGTGGACTTGTGCAGCCCGGAGGGTCTCTTCGCCTTTCATGTGCT |
| | | GCCAGCGGCTTCACCTTTTCCAGTTACCCAATGTCCTGGGTCAGACAGGCCCCC |
| | | GGCAAGGGGCTGGAGTGGGTCAGCACCATCTCTTCCGGGGGTGATACAACCTTG |
| | | TACGCAGACTCTGTTAAGGGCAGGTTCACATCCAGCCGGGACAATGCTAAGAAT |
| | | ACCCTCTATCTGCAACTCAACAGCCTGAAGACCGAGGACACCGCAATGTACTAT |
| | | TGCGCCAAACGCATTGATTGCAACAGGGGCTACTGTTACAAACGCTCTTACTGG |
| | | GGCCAGGGCACCCAGGTGACAGTCTCCTCC |
| DR595- | 1186 | CAGGTCCAGTTGCAGGAATCTGGCGGTGGCTCCGTACAGGCGGGGGGGTCCCTC |
| hIL27 | | ACGCTGTCTTGTGCCGCATCCGAATATGCTTATTCCACGTGCAACATGGGCTGG |
| Ra_VH | | TATCGCCAGGCCCCTGGAAAGGAACGCGAACTTGTGAGCGCCTTTATTAGCGAT |
| H20 | | GGTTCTACTTATTACGCTGATAGCGTGAAAGGAAGATTTACTATCACACGCGAT |
| | | AATGCTAAAAATACTGTGTACTTGCAGATGAACTCTTTGAAGCCTGAGGATACG |
| | | GCCATCTATTACTGCTCTGCAAATTGCTACCGCCGTCTGCGTAATTACTGGGGC |
| | | CAGGGCACTCAGGTCACAGTGTCAAGCGGCGGGTCAGGAGGCTCCGGCGGAAGC |
| | | GGCCAGGTCCAGCTCCAGGAGTCTGGAGGTGGCTTGGTGCAGCCTGGTGGCTCT |
| | | CTTCGTCTGTCTTGCGCTGCCTCCGGTTTCACCTTTAGTTCTTACCCTATGTCC |
| | | TGGGTGCGTCAGGCCCCCGGAAAAGGGCTGGAATGGGTGTCCACCATTTCCTCC |
| | | GGCGGTGACACTACACTGTATGCAGACAGTGTGAAGGGGAGATTCACCTCTTCC |
| | | CGTGACAACGCCAAGAACACCCTGTATCTCCAGCTCAATAGCCTGAAGACTGAG |
| | | GATACCGCAATGTACTATTGCGCCAAACGGATCGACTGTAACAGTGGATATTGT |
| | | TACAAGCGTTCCTATTGGGGCCAGGGGACCCAGGTGACTGTGAGTTCT |
| DR595- | 1187 | CAGGTGCAGCTTCAGGAGTCCGGTGGGGGTTCCGTACAGGCAGGAGGGTCCTTG |
| hIL27 | | ACCCTGAGTTGTGCGGCTTCCGAATACGCCTATAGTACATGCAACATGGGTTGG |
| Ra_VH | | TATCGCCAGGCCCCCGGTAAGGAACGCGAGTTGGTAAGCGCCTTCATCTCCGAT |
| H21 | | GGAAGTACCTACTATGCCGATTCAGTCAAGGGTCGCTTTACGATCACACGTGAC |
| | | AACGCTAAGAACACAGTCTATCTCCAGATGAATAGTCTGAAGCCCGAAGATACA |
| | | GCAATCTATTACTGTTCAGCCAACTGTTATCGGCGTTTGCGTAACTACTGGGGA |
| | | CAGGGCACCCAAGTCACCGTCTCCAGTGGTGGCGGTTCCCAGGTCCAGCTTCAG |
| | | GAATCCGGCGGTGGCTTGGTTCAGCCTGGGGGCTCCCTGCGCCTGAGCTGCGCT |
| | | GCCTCTGGATTTACCTTCTCATTGTCCAGCATGTCATGGGTCCGTCAGGGTCCC |
| | | GGCAAGGGCGTGGAGTGGGTCAGTGCCATCTCTTCCGGTGGGGCGTCAACGTAC |
| | | TATACCGATAGCGTGAAGGGACGCTTTACCATCAGCCGGGACAACGCAAAAAAT |
| | | ATGCTGTATCTCCAGTTGAACAGCCTGAAGACGGAGGACACTGCAATGTACTAT |
| | | TGCGCAAAGGGAGGGTCCGGGTACGGTGACGCCTCCAGGATGACCTCTCCTGGC |
| | | TCCCAGGGGACCCAGGTGACTGTCTCCAGC |
| DR595- | 1188 | CAGGTACAGCTTCAGGAGTCAGGGGGGGGTAGCGTCCAGGGGGGCGGTTCTTTG |
| hIL27 | | ACACTCTCCTGCGCCGCGAGCGAGTACGCATACTCTACATGCAACATGGGCTGG |
| Ra_VH | | TACAGGCAGGCCCCAGGCAAGGAAAGGGAGCTGGTGTCCGCCTTCATTAGCGAC |
| H21 | | GGAAGCACCTACTATGCTGATTCCGTGAAGGGCCGGTTCACCATCACACGCGAT |
| | | AACGCTAAGAACACGGTGTACTTGCAGATGAACTCTCTGAAGCCCGAAGACACC |
| | | GCCATCTATTACTGCTCCGCTAACTGCTATCGCAGACTGCGCAATTATTGGGGG |
| | | CAGGGCACCCAGGTGACCGTTAGCTCCGGCGGTAGCGGTGGCAGTGGGGGTTCC |
| | | GGTCAGGTGCAGCTGCAAGAGTCAGGCGGGGGTCTGGTGCAGCCGGGAGGTTCC |
| | | CTGCGCCTCTCCTGCGCCGCTTCTGGGTTTACGTTCAGCCTCTCTTCCATGTCC |
| | | TGGGTGCGCCAAGCGCCGGGCAAGGGTCTTGAGTGGGTTTCTGCGATCTCTTCC |
| | | GGTGGAGCCTCTACATACTATACAGATTCTGTGAAGGGGAGGTTCACTATCAGC |
| | | CGCGACAACGCCAAAAACATGCTGTACGTTCAGCTCAATTCCCTCAAGACTGAG |
| | | GACACAGCTATGTATTACTGTGCAAAGGGGGCAGCGGATATGGGGACCGCCTCT |
| | | CGCATGACTAGCCCTGGTTCCCAGGGGACCCAGGTGACCGTGTCCAGC |
| DR595- | 1189 | CAGGTGCAGCTGCAAGAGAGCGGCGGGGGTTCCGTACAGGCTGGTGGCTCTCTG |
| hIL27 | | ACCTTGTCTTGCGCGGCTTCAGAGTATGCCTATAGTACCTGTAATATGGGTTGG |
| Ra_VH | | TATCGCCAGGCACCGGGAAAGGAGCGGGAGCTGGTGTCTGCTTTCATCTCCGAC |
| H22 | | GGTTCTACCTACTATGCCGATTCTGTAAAGGGGAGGTTTACTATCACTCGGGAT |
| | | AACGCGAAGAACACCGTCTACCTCCAGATGAACAGCCTGAAACCCGAGGATACT |
| | | GCGATTTACTATTGCAGCGCCAACTGTTACCGGCGCTTGCGCAACTACTGGGGC |
| | | CAGGGAACTCAAGTCACTGTCAGTTCTGGCGGAGGCAGCCAGGTCCAGCTCCAG |
| | | GAGAGCGGGGGCGGATCTGTCCAAGCTGGCGGTTCCCTCCGCCTCAGCTGTCGC |
| | | GCATCTGGTAGCACTTACTCTAACTACTGTCTCGGCTGGTTTCGTCAGACAACC |
| | | GGAAAAGAGCGCGAGGGCGTGGCTGTCATCAACTGGGGCGGCGGGATGCTGTAC |
| | | TTTGCGGATAGCGTCAAGGGCCGCTTTACTGTCTCCCAGGACCAGGCAAAAAAC |
| | | ACCGTCTACCTCCAGATGAACTCTCTGAAGCCGGAGGACACCGCGATGTACTAT |
| | | TGTGCTGCCGAGAGCGTCTCCTCATTCTCTTGCGGCGGATGGCTGACCCGCCCA |
| | | GACCGGGTTCCATACTGGGGCCAGGGCACCCAGGTGACCGTTTCTTCC |
| DR595- | 1190 | CAGGTGCAGCTCCAGGAGTCTGGAGGGGGTAGCGTGCAGGCCGGAGGCTCCCTT |
| hIL27 | | ACCCTGTCCTGTGCGGCCTCCGAGTACGCTTACTCTACCTGTAACATGGGCTGG |
| Ra_VH | | TATCGGCAAGCGCCCGGCAAAGAACGCGAACTCGTGTCCGCTTTCATCAGTGAC |
| H22 | | GGTTCCACTTATTACGCTGATTCCGTTAAAGGCCGGTTTACTATCACAAGGGAT |
| | | AACGCGAAGAACACGGTGTATTTGCAGATGAACTCTTTGAAGCCAGAAGATACA |
| | | GCTATCTACTATTGCTCCGCCAACTGTTACAGGCGTCTGCGTAACTATTGGGGT |
| | | CAGGGCACCCAGGTAACAGTGTCATCTGGTGGGAGCGGCGGTTCTGGGGGGTCT |
| | | GGTCAGGTCCAGTTGCAAGAGAGCGGTGGAGGTAGCGTCCAGGCTGGTGGCTCT |
| | | CTGCGCCTGTCTTGCCGTGCCTCCGGCAGCACCTACTCCAACTATTGCTTGGGT |
| | | TGGTTCCGGCAGACAACCGGCAAAGAACGCGAGGGTGTGGCTGTTATCAACTGG |
| | | GTGGGCGGAATGCTGTACTTTGCAGATAGCGTAAAAGGCCGGTTTACCGTGAGC |
| | | CAAGACCAGGCTAAGAACACCGTGTACCTCCAGATGAATAGCTTGAAGCCCGAG |
| | | GACACGGCGATGTATTACTGTGCTGCCGAATCTGTGTCATCTTTCAGCTGCGGA |
| | | GGGTGGCTGACCAGACCAGACCGCGTCCCTTATTGGGGACAGGGCACCCAGGTC |
| | | ACCGTCTCATCT |
| DR595- | 1191 | CAGGTGCAGTTGCAGGAGAGCGGCGGGGGAAGTGTTCAGGCTGGCGGGTCCCTT |
| hIL27 | | ACACTTAGCTGCGCAGCCAGCGAGTACGCATATTCTACCTGCAACATGGGTTGG |
| Ra_VH | | TATCGCCAGGCCCCTGGAAAAGAGCGCGAACTGGTGTCCGCATTCATCTCTGAT |
| H23 | | GGCTCCACCTATTACGCTGACTCCGTGAAGGGCAGGTTCACTATCACTCGGGAT |
| | | AACGCAAAAAATACAGTGTACCTCCAAATGAACTCCTTGAAACCCGAGGATACC |
| | | GCGATTTATTACTGTAGCGCCAACTGTTACCGCAGACTGAGAAACTACTGGGGG |
| | | CAGGGCACACAGGTGACCGTAAGCTCCGGCGGGGGCAGTCAGGTGCAGCTCCAG |
| | | GAGAGTGGCGGAGGTTCCGTGCAGGCTGGAGGCTCACTGAGGTTGTCTTGCCGT |
| | | GCCTCCCGCAGTCCCTACGGTAATTACTGCCTCGGGTGGTTCCGCCAGTCCACG |
| | | GGTAAGGAGAGAGAGGGTGTGGCCGTTATCAACTGGGTCGGGGGTATGCTGTAT |
| | | TTTGCCGACAGCGTCAAGGGACGCTTCACCGTAAGCCAGGACCACGCTAAGAAT |
| | | ACCGTCACTCTGCAAATGAACTCCCTGAAGCCTGAGGATACGGCTATGTACTAT |
| | | TGTGCAGCGGAGAGCGTGAGTTCCTTCTCTTGTGGCGGGTGGCTGACCAGACCA |
| | | GACCGGGTGCCCTACTGGGGCCAGGGCACTCAAGTTACCGTCTCATCC |
| DR595- | 1192 | CAGGTGCAACTTCAGGAATCCGGCGGTGGCAGCGTTCAGGCCGGTGGCTCCCTG |
| hIL27 | | ACCCTGTCCTGTGCAGCGAGCGAATATGCGTACTCTACGTGCAATATGGGCTGG |
| Ra_VH | | TATCGTCAGGGTCCAGGCAAGGAGCGCGAATTGGTGAGTGCGTTCATCTCCGAT |
| H23 | | GGGTCAACATACTATGCGGACTCTGTGAAGGGCCGCTTTACCATCACCAGAGAC |
| | | AACGCCAAGAACACGGTCTACCTGCAAATGAACAGCCTGAAGCCCGAGGACACC |
| | | GCAATCTACTATTGTAGTGCTAATTGTTACCGCAGGCTGAGGAATTACTGGGGT |
| | | CAAGGCACCCAAGTCACCGTGTCCAGCGGGGGTTCCGGCGGTAGCGGAGGCTCC |
| | | GGCCAGGTGCAGCTCCAGGAGAGTGGGGGAGGGAGCGTGCAAGCGGGAGGGAGT |
| | | CTCAGGCTTTCATGCCGTGCTTCTCGCAGCCCTTATGGCAACTATTGTCTGGGC |
| | | TGGTTCAGGCAGAGTACAGGCAAGGAACGCGAGGGCGTGGCAGTCATCAACTGG |
| | | GTCGGCGGGATGTTGTACTTCGCTGACTCCGTGAAGGGCCGCTTTACAGTCTCC |
| | | CAAGACCACGCCAAGAACACCGTCACGCTCCAGATGAACAGTCTCAAGCCCGAA |
| | | GATACTGCCATGTATTACTGGGCTGCGGAGTCAGTGTCCTCTTTCAGCTGTGGG |
| | | GGTTGGCTGACTCGCCCAGACCGGGTGCCATATTGGGGCCAGGGTACACAAGTC |
| | | ACCGTCAGCAGC |
| DR595- | 1193 | CAAGTCCAGTTGCAGGAGAGCGGGGGCGGAAGCGTGCAGGCTGGAGGGAGCCTC |
| hIL27 | | ACCCTGTCCTGCGCCGCTTCCGAGTACGCCTACTCTACTTGTAACATGCGGGGG |
| Ra_VH | | TATAGGCAGGCTCCCGGCAAAGAACGCGAGCTGGTCTCCGCGTTTATTTCTGAC |
| H24 | | GGATGTACCTATTACGCGGACAGTGTGAAGGGCAGATTTACAATCACTAGGGAT |
| | | AACGCAAAAAACACTGTCTATCTCCAGATGAACTCTTTGAAGCCTGAGGACACA |
| | | GCTATCTATTACTGTTCCGCGAACTGCTACAGACGCCTCCGTAACTATTGGGGT |
| | | CAGGGTACTCAGGTGACTGTTAGTTCAGGTGGGGGCTCCCAGGTCCAGCTTCAA |
| | | GAATCTGGGGGTGGATTGGTCCAGCCTGGCGGTTCTCTGCGCCTGTCTTGTGCT |
| | | GCCAGTGGCTTCACTTTTTCCCACAGCGGCATGTCCTGGGTGCGCCAGGGTCCA |
| | | GGCAAGGGCCTCGAATGGGTTAGTACCATCAACAGCGGAGGTGCCAGCACCTAC |
| | | TATACAGATTCTGTGAAGGGCAGATTCACTATTAGTCGCGACAATGCGAAGAAC |
| | | ATGCTTTACTTGCAGCTGAACTCCCTGAAGACCGAAGACACTGCAATGTATTAC |
| | | TGCGCCAAGGGCGGTTCTGGCTACGGTGATGCGTCTCGCATGACATCCCCAGGG |
| | | AGTCAGGGAACCCAGGTGACCGTGAGTTCA |
| DR595- | 1194 | CAGGTCCAGCTCCAGGAATCTGGAGGGGGTAGCGTACAAGCTGGCGGTTCTCTG |
| hIL27 | | ACTCTGAGCTGCGCTGCCTCCGAGTACGCCTACTCAACTTGCAATATGGGCTGG |
| Ra_VH | | TATAGGCAAGCACCAGGGAAGGAAAGAGAATTGGTTTCCGCCCTCATCTCCGAC |
| H24 | | GGAAGTACTTATTACGCCGATTCCGTGAAGGGACGCTTCACGATTACACGGGAC |
| | | AACGCAAAGAACACCGTGTATGTCCAGATGAACAGCCTGAAGCCTGAGGACACC |
| | | GCAATTTATTACTGTTCCGCCAACTGCTACAGACGCTTGCGCAACTACTGGGGT |
| | | CAAGGCACCCAGGTGACCGTCTCTAGTGGAGGCTCCGGTGGCTCCGGGGGCTCT |
| | | GGACAGGTGCAGCTTCAGGAGAGTGGTGGCGGGCTGGTGCAGCCTGGGGGTTCC |
| | | CTTCGTCTCTCTTGTGCAGCCAGCGGATTCACATTTAGTCACAGCGGCATGAGT |
| | | TGGGTTCGGCAAGCACCTGGGAAGGGGCTGGAGTGGGTATCCACAATCAACAGG |
| | | GGAGGTGCCAGCACGTACTATACCGACTCAGTTAAAGGGCGCTTCACCATTAGT |
| | | CGCGACAACGCGAAGAATATGCTGTACTTGCAGCTGAACAGCCTGAAGACCGAG |
| | | GACACTGCCATGTACTATTGCGCCAAAGGGGGCAGCGGCTACGGAGATGCGAGC |
| | | CGTATGACTTCACCTGGGAGCCAGGGCACCCAGGTGACCGTCTCCTCT |
| DR596- | 1195 | CAGGTGCAGCTTCAGGAGTCTGGGGGAGGCCTGGTACAACCCGGTGGCAGTCTC |
| hIL27 | | AGGCTCAGCTGCACAGCCTCCGGCCTGACCTTCGACGATTCCGTGATGGGCTGG |
| Ra_VH | | TTTAGGCAGGCCCCAGGTAAAGGCCGGGAGGCTGTAAGCTGCATTAGCTCCTCC |
| H1 | | GGAGCCAACGCCTTCTACGCTGATTCTGTGAAGGGGCGTTTTACCATTTCTCGC |
| | | GACAACGCCAAGAACACCTTGTATTTGCAGATGAACTCCCTGAAACCCGAGGAC |
| | | ACCGCCACCTACTATTGTAAGCGTGGCCACGCTTGTGGGGGTTATTACCCCATT |
| | | CCCTACGATGACTATTGGGGTCAGGGTACGCAAGTCACCGTATCTTCCGGGGGA |
| | | GGCTCCCAGGTGCAGCTCCAGGAGTCAGGGGGGGGACTGGTGCAGCCCGGAGGT |
| | | TCCCTGCGGCTGTCTTGTGCGGCATCCGGCTTTACTTTGTCTTCCTACCCCATG |
| | | AGCTGGGTCCGCCAAGCGCCAGGGAAGGGTCTGGAGTGGATTTCTACAATCTCA |
| | | GCTGGAGGCGACACTACCCTCTACGCTGACTCTGTTAAAGGCCGCTTCACTAGC |
| | | AGTCGGGATAACGCCAAGAACACGCTGTACCTCCAGCTTAATAGGTTGAAAACC |
| | | GAGGACGCGGCCATCTATTACTGCGCCAAGCGTATTGACTGTAACTCTGGATAC |
| | | TGCTATCGTCGCAACTATTGGGGTCAGGGGACCCAAGTTACAGTGAGTTCT |
| DR596- | 1196 | CAGGTTCAGCTTCAGGAGAGTGGGGGGGGCCTGGTTCAGCCCGGGGGTTCCCTT |
| hIL27 | | CGCCTGAGCTGTACGGCAAGCGGTCTGACATTTGACGATTCCGTGATGGGCTGG |
| Ra_VH | | TTTCGTCAGGCTCCTGGCAAGGGTAGGGAGGGGGTGAGTTGCATCAGCTCCAGG |
| H1 | | GGAGCCAACGCCTTTTACGCGGATAGCGTCAAGGGAAGGTTTACCATCAGCCGG |
| | | GACAATGCTAAGAACACCCTGTACTTGCAGATGAACAGTCTGAAACCAGAGGAC |
| | | ACCGCAACTTATTACTGCAAGCGCGGCCACGCTTGCGCGGGCTATTACCCGATT |
| | | CCCTATGACGATTACTGGGGCCAGGGCACTCAGGTGACAGTCAGCTCTGGCGGA |
| | | AGTGGGGGAAGCGGGGGTTCTGGCCAGGTTCAGTTGCAGGAGAGCGGAGGTGGC |
| | | CTGGTCCAGCCCGGTGGCTCTCTGCGCCTGAGCTGCGCCGCTTCCGGTTTCACT |
| | | TTCTCTAGCTATCCCATGAGTTGGGTGCGTCAGGCTCCAGGCAAGGGACTGGAG |
| | | TGGATCAGTACCATCTCAGCTGGGGGCGACACCACATTGTACGCCGATTCTGTG |
| | | AAGGGCAGATTTACCTCCTCTCGCGATAATGCCAAGAACACCCTGTATCTCCAG |
| | | TTGAACTCTCTGAAGACCGAGGACGCTGCCATCTACTATTGCGCCAAGCGTATC |
| | | GACTGTAATTCCGGCTACTGTTACCGTCGGAACTACTGGGGACAAGGCACCCAG |
| | | GTTACCGTTAGCTCC |
| DR596- | 1197 | CAGGTCCAACTGCAAGAATCCGGTGGGGGTCTGGTTCAGCCCGGTGGCTCACTG |
| hIL27 | | CGCCTGTCATGTACCGCCAGCGGCCTGACTTCGATGACAGTTGTCATGGGATGG |
| Ra_VH | | TTCCGCCAGGCCCCTGGAAAGGGAAGGGAAGCGGTCAGCTGTATTTCTTCCAGC |
| H2 | | GGGGCTAATGCCTTCTACGCTGACTCCGTGAAAGGTCGGTTTACCATCAGTAGG |
| | | GATAATGCCAAGAACACCTTGTACTTGCAGATGAACTCCCTGAAACCTGAGGAC |
| | | ACCGCCACCTATTACTGTAAGCGTGGCCACGCCTGCGCTGGCTACTATCCCATC |
| | | CCATACGATGACTACTGGGGCCAGGGTACTCAGGTAACTGTTAGCTCTGGAGGG |
| | | GGCTCCCAGGTGCAGCTTCAGGAATCTGGTGGCGGACTGGTCCAGCCTGGGGGT |
| | | TCCCTTCGTCTGAGCTGCGCAGCCTCTGGTTTCACTTTTAGCCTGAGCGGTATG |
| | | AGCTGGGTGAGGCAGGCCCCTGGCAAGGGTCTGGAGTGGGTCAGCGCGATCTCC |
| | | AGCGGCGGTGCCTCTACCTACTATACAGATTCCGTGAAGGGCAGATTCACTATC |
| | | TCCAGAGATAACGCTAAAAACATCCTCTACCTGCAACTGAACTCCCTCAAGACC |
| | | GAGGATACTGCCATGTATTACTGTGCAAAGGGAGGCTCTGGTTATGGTGATGCC |
| | | AGTCGGATGACCTCCCCTGGCTCCCAGGGAACACAGGTTACGGTCTCATCC |
| DR596- | 1198 | CAGGTCCAGCTTCAGGAGAGTGGGGGGGGACTGGTACAGCCCGGAGGCTCCCTG |
| hIL27 | | AGACTGAGTTGCACCGCCAGTGGTCTGACATTCGATGACAGCGTGATGGGCTGG |
| Ra_VH | | TTCAGACAGGCCCCCGGCAAAGGCCGCGAGGCCGTTAGCTGTATCTCTAGTAGC |
| H2 | | GGGGCCAACGCTTTTTACGCTGACTCTGTTAAGGGCAGGTTCACCATTTCCCGC |
| | | GACAATGCGAAGAACACCCTGTACTTGCAGATGAACAGTCTGAAACCCGAAGAC |
| | | ACGGCTACTTACTATTGTAAGAGGGGTCACGCCTGCGCCGGTTATTACCCCATT |
| | | CCCTACGATGACTATTGGGGGCAAGGGACCCAGGTCACAGTCTCCAGGGGGGGG |
| | | TCCGGCGGTAGCGGGGGCTCCGGCCAGGTGCAGCTGCAAGAGTCGGGGGGCGGT |
| | | CTTGTTCAGCCAGGTGGCTCTCTGCGGCTGTCCTGCGCGGCCTCTGGATTCACA |
| | | TTCTCTCTCAGTGGCATGTCTTGGGTGCGCCAGGCTCCGGGCAAGGGCCTCGAA |
| | | TGGGTGTCCGCCATCTCCAGCGGTGGCGCTTCCACTTATTACACCGATTGTGTG |
| | | AAGGGACGCTTCACCATCTCTAGGGACAACGCGAAGAACATTTTGTATCTCCAA |
| | | CTGAACAGTCTTAAAACCGAGGACACTGCCATGTATTACTGTGCTAAGGGTGGG |
| | | TCTGGCTACGGAGATGCGTCCAGGATGACGAGCCCAGGCTCCCAGGGCACCCAG |
| | | GTGACCGTGTCCTCT |
| DR596- | 1199 | CAGGTCCAGGTCCAGGAGTCCGGTGGGGGCCTCGTTCAGCCCGGGGGATCTCTG |
| hIL27 | | AGGCTGAGCTGTACCGCCTCTGGCCTGACCTTCGACGATTCCGTGATGGGCTGG |
| Ra_VH | | TTCAGACAGGCTCCCGGCAAGGGCCGCGAAGCTGTCTCGTGCATCTCCTCTTCC |
| H3 | | GGCGCAAATGCGTTCTACGCCGACTCAGTGAAGGGCCGTTTTACCATTTCTCGG |
| | | GATAACGCTAAAAACACGCTGTATCTGCAAATGAACTCGTTGAAACCGGAGGAT |
| | | ACCGCCACCTATTACTGCAAACGCGGTCATGCCTGTGCTGGGTATTACCCAATC |
| | | CCTTACGATGACTATTGGGGCCAAGGCACCCAGGTCACCGTCAGTAGCGGCGGT |
| | | GGCTCCCAGGTTCAGTTGCAGGAATCAGGGGGGGGCTCCGTCCAAGCGGGAGGC |
| | | TCTCTGCGTCTGTCATGTGTAGCCAGTGGCTACGTGTCATGCGATTACCTCCTG |
| | | CCCTCCTGGTATAGACAGGCTCCCGGCAAGGAGCGCGAGTTCGTGAGCATTATC |
| | | GACGGCACGGGTTCCACGTCCTACGCTGCCCCCGTTAAGGGTCGCTTTACAGCT |
| | | AGTGAGGACAAGGGCAAGAACATTGCGTACCTCCAGATGAACCCCCTGAAGCCC |
| | | GAAGATACAGCCATGTACTATTGCAAGGCCAGGTGTGTGAGGGGCCGCGCTGTT |
| | | TCAGAATACTGGGGCCAGGGCACCCAGGTCACTGTTTCTTCT |
| DR596- | 1200 | CAGGTTCAGTTGCAGGAATCCGGGGGGGGATTGGTCCAGCCAGGAGGCTCCCTT |
| hIL27 | | CGCCTGAGCTGCACTGCCTCTGGACTGACATTTGACGATTCTGTTATGGGCTGG |
| Ra_VH | | TTCCGCCAAGCACCAGGAAAGGGCAGAGAGGCAGTGTCTTGTATCTCTTCCAGC |
| H3 | | GGAGCCAATGCGTTTTATGCTGACAGCGTGAAGGGCAGGTTCACCATCTCACGC |
| | | GACAACGCTAAGAACACCCTCTATCTTCAGATGAACTCTCTCAAACCTGAGGAT |
| | | ACCGCCACCTATTACTGTAAACGCGGACATGCTTGCGCCGGTTACTATCCTATG |
| | | CCCTATGATGACTACTGGGGGCAGGGCACCCAGGTGACCGTGTCAAGCGGGGGA |
| | | TCAGGAGGCAGCGGAGGCAGCGGCCAGGTCCAACTTCAGGAGTCAGGAGGCGGT |
| | | TCTGTGCAGGCCGGAGGCTCACTGCGGCTGTCCTGTGTGGCGAGTGGCTACGTA |
| | | AGCTGCGACTACTTCGTGCCTTCTTGGTACAGGCAGGCCCCTGGCAAAGAGAGG |
| | | GAATTTGTGTCCATCATTGATGGTACAGGCAGCACCTCCTATGGGGCCTCCGTT |
| | | AAGGGCAGATTTACTGCCTCCGAGGACAAGGGCAAGAATATCGCCTATCTCCAG |
| | | ATGAACTCCGTGAAGCCGGAAGATACCGCAATGTACTATTGCAAAGCCAGTTGT |
| | | GTGAGAGGGAGAGCGGTAAGTGAGTATTGGGGTCAGGGCACTCAGGTGACGGTT |
| | | TCCAGC |
| DR596- | 1201 | CAGGTGCAACTTCAGGAGTCTGGAGGGGGACTGGTCCAGCCTGGGGGCTCTCTG |
| hIL27 | | CGTCTGTCTTGCACCGCCTCCGGCCTGACGTTTGATGACTCAGTCATGCGGGGG |
| Ra_VH | | TTCCGCCAAGCCCCTGGGAAGGGCAGGGAGGCCGTCAGCTGCATCTCTAGCTCC |
| H4 | | GGGGCCAACGCTTTCTACGCGGATAGCGTCAAGGGCCGGTTCACCATCAGTAGA |
| | | GATAACGCAAAAAACACTTTGTATCCCCAGATGAACAGCCTGAAACCCGAGGAT |
| | | ACCGCAACGTATTACTGTAAGCGCGGACACGCTTGTGGGGGCTATTACCCTATT |
| | | CCCTATGATGACTACTGGGGTCAGGGCACTCAAGTCACCGTCAGTTCTGGCGGA |
| | | GGTTCTCAGGTACAGGTCCAGGAGTCTGGGGGGGGCCTGGTGCAGCCCGGCGAA |
| | | TCTCTGAGACTGTCATGCACCGCATCCGGCTTTACCTTCTCTAATTACGCCATG |
| | | AGCTGGGTCAGACAGGCCCCTGGTAAGGGGCTGGAGTGGGTCAGTGGAATCAAC |
| | | GTGGCCTATGGGATCACCTCCTACGCGGACTCCGTCAAGGGTAGATTTACAATC |
| | | TCTCGCGACAACACCAAGAACACCCTGTACCTTCAGCTGAACTCACTCAAAACC |
| | | GAAGACACCGCGATCTATTACTGTGTGAAACACTCAGGTACAACCATCCCCAGA |
| | | GGATTCATCTCATACACAAAACGCGGCCAGGGTACTCAGGTAACCGTGTCCTCA |
| DR596- | 1202 | CAGGTCCAGCTCCAGGAGAGCGGGGGGGGCCTGGTGCAGCCTGGCGGTTCCCTG |
| hIL27 | | CGGCTCAGCTGTACCGCCTCCGGGCTGACGTTCGACGATTCTGTGATGGGCTGG |
| Ra_VH | | TTTCGCCAGGCCCCCGGCAAGGGACGCGAAGCTGTAAGCTGCATTAGTTCCTCT |
| H4 | | GGAGCGAATGCCTTCTACGCCGACTCTGTGAAGGGCCGCTTCACAATCTCCCGC |
| | | GACAACGCTAAGAACACGCTGTACCTTCAGATGAACTCTCTGAAACCCGAGGAT |
| | | ACTGCCACCTACTATTGCAAGCGCGGCCATGCCTGTGCTGGGTACTATCCAATC |
| | | CCGTATGACGATTACTGGGGCCAAGGGACCCAGGTCACCGTAAGTTCAGGAGGT |
| | | AGCGGTGGCTCTGGGGGCTCTGGTCAAGTACAGTTGCAGGAAAGTGGGGGGGGT |
| | | CTGGTTCAGCCAGGCGAGTCATTGAGATTGTCTTGTACGGCTAGTGGATTCACA |
| | | TTTAGTAACTACGCCATGAGCTGGGTCCGTCAAGCACCTGGCAAGGGCCTGGAG |
| | | TGGGTATCCGGTATTAACGTGGCCTACGGAATTACAAGTTATGCAGACCCTGTG |
| | | AAGGGCAGGTTCACAATCTCTCGGGACAACACCAAGAACACGCTGTACCTTCAG |
| | | CTGAACTCTCTGAAAACCGAAGATACTGCTATCTATTACTGCGTGAAGCACAGC |
| | | GGCACAACCATCCCACGCGGCTTCATCTCTTATACCAAGAGAGGCCAGGGCACC |
| | | CAAGTCACCGTGTCATCC |
| DR596- | 1203 | CAGGTGCAACTCCAGGAGTCCGGCGGTGGGCTCGTGCAACCGGGTGGCTCCTTG |
| hIL27 | | AGACTGTCTTGTACCGCCAGCGGACTTACCTTCGACGATTCTGTGATGGGCTGG |
| Ra_VH | | TTCCGGCAAGCGCCGGGCAAGGGCAGAGAGGCCGTGAGCTGCATCTCTAGTTCT |
| H5 | | GGTGCTAACGCTTTTTACGCCGACTCCGTGAAAGGCCGTTTCACGATTTCAAGA |
| | | GATAACGCCAAAAACACTCTCTACCTCCAGATGAACTCCCTCAAGCCTGAGGAC |
| | | ACTGCCACCTATTACTGTAAGCGGGGCCACGCTTGCGCAGGCTATTACCGTATT |
| | | CCCTATGACGATTACTGGGGCCAGGGTACACAGGTGACGGTGTCCAGTGGCGGA |
| | | GGGTCACAAGTTCAACTTCAGGAGAGCGGCGGAGGGTCTGTCCAGGCTGGTGGC |
| | | TCCCTGCGTCTCTCCTGTACCGCCTCCGGCTACGTGTCATGTGATTACTTTCTG |
| | | CCTTCCTGGTACAGGCAAGCACCCGGTAAGGAGCGCGAATTTGTCAGCGTTATT |
| | | GACGGCACGGGTAGTACGTCCTATGCTGCCTCCGTGAAGGGCCGGTTCACGGCA |
| | | TCCCAGGACAAGGGGAAAAATATCGCATACCTCCAAATGAACAGTCTGAAGCCG |
| | | GAGGATACAGCTATGTATTACTGCAAGGCTTCATGTGTGCGCGGAAGAGCTATT |
| | | AGTGAGTATTGGGGCCAGGGTACACAGGTAACGGTCAGCTCC |
| DR596- | 1204 | CAGGTCCAGTTGCAGGAGAGGGGCGGTGGCTTGGTGCAGCCCGGTGGGTCACTG |
| hIL27 | | AGACTGTCCTGCACTGCAAGCGGCCTTACGTTCGATGACAGCGTGATGGCGGGG |
| Ra_VH | | TTCCGCCAGGCACCCGGCAAGGGACGTGAGGCCGTGTCCTGCATTAGCTCATCC |
| H5 | | GGCGCTAATGCCTTTTATGCCGACAGCGTCAAGGGCAGATTTACCATCCCTCGC |
| | | GACAATGCCAAAAATACCCTGTATCTTCAAATGAATAGCCTGAAGCCAGAGGAT |
| | | ACCGCTACTTACTATTGTAAGCGCGGCCACGCCTGCGCGGGCTATTACCCTATC |
| | | CCCTACGATGACTATTGGGGCCAGGGTACTCAAGTGACTGTGAGTTCCGGTGGC |
| | | TCCGGCGGGTCTGGAGGCTCAGGTCAAGTCCAGTTGCAGGAGTCCGGGGGCGGT |
| | | AGCGTGCAGGGGGGCGGTTCCGTGCGCCTGTCTTGTACCGCATCTGGGTACGTC |
| | | TCCTGCGATTACTTCCTGCCTTCCTGGTACAGGCAGGCCCCTGGCAAGGAGCGT |
| | | GAGTTTGTGTCCGTGATCGACGGGACAGGTTCCACCTCCTACGCTGCATCCGTT |
| | | AAGGGTCGGTTTACAGCCAGCCAGGACAAAGGAAAGAACATCGCTTACCTGCAA |
| | | ATGAACTCCCTGAAACCTGAGGATACCGCTATGTATTACTGCAAGGCCTCCTGG |
| | | GTCCGTGGCCGTGCGATCTCCGAATACTGGGGCCAGGGCACCCAGGTGACTGTT |
| | | AGCAGC |
| DR596- | 1205 | CAGGTGCAGTTGCAGGAGTCTGGGGGAGGTCTCGTGCAGCCAGGAGGGTCACTC |
| hIL27 | | CGCCTGAGTTGTACTGCCAGCGGTTTGACATTCGACGATTCTGTCATGGGCTGG |
| Ra_VH | | TTTCGCCAAGCTCCAGGGAAGGGCCGCGAGGCTGTGTCCTGCATCTCATCCTCT |
| H6 | | GGGGCCAACGCTTTTTACGCTGATTCTGTCAAAGGCCGGTTCACAATCAGCCGG |
| | | GACAACGCCAAGAACACTTTGTATCTCCAAATGAACAGCCTGAAGCCTGAGGAG |
| | | ACTGCAACCTATTACTGTAAGCGCGGACACGCCTGCGCTGGCTATTACCCCATC |
| | | CCTTACGACGATTATTGGGGTCAAGGAACTCAGGTTACTGTTAGGTCCGGGGGG |
| | | GGCAGCCAGGTGCAGCTTCAGGAGTCAGGTGGAGGTCTGGTGCAACCTGGAGGC |
| | | TCCCTGCGCCTGTCCTGTGCCGCATCCGGCTTTAGTTTCTCTAGCTACGCAATG |
| | | AAATGGGTGCGTCAAGCCCCAGGCAAAGGTCTGGAGTGGGTGTCCACCATCAGT |
| | | AGCGGTGGCTCCAGCACGAACTACGCCGACTCCGTGAAAGGCCGGTTCACCATC |
| | | AGCCGTGATAACGCTAAGAACACGCTGTACCTCCAGCTGAACTCTCTGAAGATC |
| | | GAGGACACGGCAATGTATTACTGTGCAAAGGCAATCGTTCCAACTGGTGCCACT |
| | | ATGGAGAGGGGGCAGGGAACCCAAGTCACCGTTAGCTCC |
| DR596- | 1206 | CAAGTGCAGCTCCAAGAAAGCGGCGGAGGCCTGGTGCAGCCTGGCGGTTCTCTG |
| hIL27 | | AGACTGAGCTGTACTGCCTCAGGTCTGACTTTTGACGATTCTGTCATGGGATGG |
| Ra_VH | | TTTAGACAAGCTCCAGGCAAGGGTCGTGAGGCGGTGAGGTGTATTAGCTCCTCC |
| H6 | | GGCGCGAACGCATTTTATGCCGATTCCGTTAAGGGTCGCTTCACAATCTCACGG |
| | | GACAACGCGAAGAATACACTGTACCTGCAAATGAACTCCCTGAAGCCCGAGGAC |
| | | ACAGCTACCTATTACTGCAAGCGCGGCCACGCGTGTGCAGGTTATTACCCTATT |
| | | CCCTATGATGACTACTGGGGACAAGGGACTCAGGTTACCGTCAGCTCCGGTGGC |
| | | AGCGGCGGATCTGGTGGCTCCGGCCAGGTGCAGCTTCAGGAGTCCGGGGGAGGC |
| | | CTGGTGCAGCCGGGGGGTTCATTGCGCGTTTCTTGCGCCGCGAGTGGCTTCTCC |
| | | TTTTCCAGCTATGCCATGAAGTGGGTCCGCCAGGCTCCTGGGAAGGGCCTGGAG |
| | | TGGGTGTCCACCATCTCCTCTGGGGGCTCCAGTACCAACTACGCCGACAGCGTG |
| | | AAGGGTCGCTTCACTATCAGCAGAGATAACGCTAAGAACACACTGTACCTCCAG |
| | | CTGAATAGCCTGAAGATCGAGGATACGGCTATGTATTACTGTGCCAAGGCGATT |
| | | GTGCCTACAGGAGCCACTATGGAACGCGGCCAAGGCACCCAGGTGACGGTGTCA |
| | | AGC |
| DR596- | 1207 | CAGGTGCAACTTCAAGAATCTGGAGGTGGACTGGTCCAGCCTGGGGGTTCTCTG |
| hIL27 | | CGCCTCTCTTGTACTGCTTCCGGCCTCACATTTGACGATTCTGTTATGGGATGG |
| Ra_VH | | TTCAGACAGGCCCCAGGTAAGGGTCGCGAGGCCGTGAGTTGTATCAGCCCCAGC |
| H7 | | GGCGCTAACGCATTCTATGCCGACAGTGTCAAGGGACGGTTTACAATCTCCAGG |
| | | GACAACGCCAAAAACACGCTCTACCTTCAGATGAACTCACTGAAGCCCGAGGAT |
| | | ACTGCTACCTATTACTGCAAACGCGGTCATGCTTGTGCCGGTTATTACCCCATC |
| | | CCATACGATGACTACTGGGGCCAGGGGACCCAGGTAACCGTCAGGTCCGGCGGT |
| | | GGATCACAGGTGCAGCTTCAGGAGAGTGGTGGAGGTCTCGTGCAACCTGGAGGT |
| | | AGTCTCCGCTTGTCTTGTGCTGCCTCCGGTTTCACCTTTTCCAGCTACCCCATG |
| | | AGTTGGGTGCGCCAAGCGCCGGGGAAGGGCCTGGAGTGGATTTCAACTATCAGC |
| | | GCTGGAGGCGATACCACACTGTACGCCGATAGTGTTAAGGGAAGGTTCACTTCC |
| | | TCTAGGGATAACGCCAAGAACACCCTGTATCTTCAGCTGAACAGTCTGAAGACG |
| | | GAGGACACTGCTATCTATTACTGCGCAAAACGCATTGATTGCAACTCCGGCTAT |
| | | TGCTACCGCCGGAACTACTGGGGCCAGGGGACCCAGGTGACAGTCAGTTCC |
| DR596- | 1208 | CAGGTACAGGTCCAGGAGAGCGGCGGTGGCCTGGTACAGCCAGGGGGTTCATTG |
| hIL27 | | CGTCTGAGCTGCACTGCTTCTGGTCTGACGTTTGATGACTCTGTTATGGGCTGG |
| Ra_VH | | TTCCGCCAAGCGCCCGGCAAGGGACGGGAAGCTGTTAGGTGTATCTCCAGTTCC |
| H7 | | GGGGCCAACGCCTTCTACGCCGATTCTGTGAAGGGTCGGTTCACTATCTCACGC |
| | | GACAACGCCAAGAACACCCTGTACCTTCAGATGAACAGTCTGAAGCCTGAGGAT |
| | | ACCGCCACCTACTATTGCAAGCGCGGCCATGCGTGCGCGGGCTATTACCCTATC |
| | | CCTTACGATGACTATTGGGGGCAGGGCACCCAGGTGACTGTGTCCAGTGGAGGC |
| | | TCCGGTGGCAGTGGAGGGTCCGGCCAGGTCCAACTCCAGGAGTCTGGTGGGGGC |
| | | TTGGTCCAGCCTGGGGGTAGCTTGCGCCTGTCTTGCGCTGCCTCCGGGTTCACC |
| | | TTCTCTAGTTATCCTATGTCTTGGGTGCGGCAAGCGCCGGGTAAGGGCCTGGAG |
| | | TGGATTTCTACTATCTCTGCTGGCGGTGACACCACGTTGTACGCAGATTCCGTG |
| | | AAGGGGCGTTTCACCTCCTCAAGAGACAATGCGAAGAACACCTTGTACCTCCAG |
| | | CTGAACAGCCTGAAGACCGAGGACACCGCTATTTACTATTGTGCCAAGCGTATT |
| | | GATTGTAATAGCGGTTATTGCTACCGCAGGAACTACTGGGGCCAGGGCACACAG |
| | | GTCACCGTGAGCAGT |
| DR596- | 1209 | CAGGTGCAACTCCAGGAAAGCGGGGGCGGGCTTGTGCAGCCCGGCGGTAGCCTG |
| hIL27 | | CGGCTGAGTTGTACTGCAAGCGGTCTCACCTTCGACGATAGTGTAATGGGTTGG |
| Ra_VH | | TTTCGTCAAGCACCCGGAAAGGGAAGGGAGGCGGTGTCTTGCATCTCCAGCAGT |
| H8 | | GGCGCGAATGCGTTTTATGCCGATTCCGTCAAGGGGCGGTTTACGATCTCCAGG |
| | | GATAACGCCAAGAACACGCTGTACCTCCAGATGAACAGTCTGAAGCCCGAGGAT |
| | | ACGGCCACATATTACTGTAAGCGCGGGCACGCCTGTGCCGGATACTATCCCATC |
| | | CCTTATGACGATTATTGGGGTCAAGGTACGCAGGTCACAGTTAGCTCTGGCGGA |
| | | GGTTCCCAGGTCCAACTTCAGGAGTCCGGTGGAGGGTCCGTTCAGGTGGGCGGA |
| | | TCTCTTCGCCTGAGCTGCGCGGCCAGCGGATTTACCTTCAGCCCCTACCCAATG |
| | | AGTTGGGTGCGTCAGGCTCCGGGTAAGGGCCTGGAGTGGATCTCTACCATTAGT |
| | | GCTGGCGGAGACACCACGCTGTACGCCGACAGCGTGAAGGGGCGCTTCACCTCC |
| | | TCCCGCGATAACGCAAAGAATACCCTGTACCTCCAGCTCAATTCCTTGAAGACC |
| | | GAAGATACGGCCATTTATTACTGCGCCAAGCGCATTGATTGCAATAGCGGCTAT |
| | | TGCTATCGGAGGAACTACTGCGGGACAGGGAACCCAAGTCACTGTTTCATCC |
| DR596- | 1210 | CAGGTCCAGCTCCAAGAGTCAGGAGGCGGACTCGTGCAACCGGCGGGCTCCCTG |
| hIL27 | | AGGCTGTCTTGTACCGCAAGCGGCCTGACCTTCGATGACAGTGTCATGGGCTGG |
| Ra_VH | | TTCAGGCAGGCCCCCGGCAAAGGCCGTGAGGCCGTCAGCTGTATCAGTTCTAGT |
| H8 | | GGAGCCAACGCCTTTTACGCCGATTCCGTAAAGGGCCGTTTCACCATTTCAAGG |
| | | GACAATGCCAAGAATACCCTGTATCTGCAAATGAACTCCCTCAAGCCCGAAGAT |
| | | ACCGCCACCTACTATTGCAAGCGCGGACATGCCTGTGCCGGTTATTACCCCATT |
| | | CCCTATGACGATTATTGGGGTCAGGGAACCCAGGTGACCGTGTCCAGTGGAGGG |
| | | TCCGGGGGCTCTGGGGGGTCCGGCCAGGTGCAGTTGCAGGAGTCAGGGGGTGGG |
| | | AGCGTCCAGGTTGGGGGTAGTCTGCGCCTGTCTTGTGCAGCCTCTGGGTTCACC |
| | | TTCAGCTCCTACCCCATGAGCTGGGTGCGCCAGGCTCCGGGAAAAGGTCTTGAG |
| | | TGGATCTCTACAATCTCCGCGGGTGGCGACACTACCCTGTACGGTGACAGCGTG |
| | | AAGGGCCGGTTTACAAGTTCTCGCGACAACGCCAAGAACACCTTGTACCTCCAG |
| | | CTCAACTCCCTGAAGACCGAGGATACCGCCATTTATTACTGTGCAAAAAGGATT |
| | | GATTGTAACTCCGGCTACTGTTATCGCAGAAATTACTGGGGCCAGGGGACCCAG |
| | | GTTACAGTGTCTAGC |
| DR596- | 1211 | CAGGTTCAGCTGCAAGAGAGGGGAGGGGGTCTCGTGCAGCCGGGTGGGAGCCTG |
| hIL27 | | CGCCTTTCCTGTACTGCTTCTGGACTGACCTTCGATGACTCCGTGATGGGTTGG |
| Ra_VH | | TTCCGGCAGGCCCCTGGAAAGGGCCGTGAAGCTGTGTCCTGTATCAGCTCTTCC |
| H9 | | GGGGCGAATGCTTTCTACGCCGATAGCGTTAAGGGCCGCTTTACCATCTCCCGC |
| | | GACAACGCAAAGAATACTCTGTACTTGCAGATGAACAGCCTCAAGCCCGAAGAC |
| | | ACCGCCACTTATTACTGTAAGCGCGGCCACGCTTGCGCGGGTTACTATCCCATC |
| | | CCTTACGATGACTACTGGGGACAGGGAACCCAGGTAACCGTGTCATCTGGCGGG |
| | | GGCAGTCAGGTCCAGTTGCAGGAAAGCGGAGGTGGCAGCGTTCAGAGTGGCGGT |
| | | TCCCTGCGTGTTAGCTGTGCAGCCTCCGGCTTTACATACAGCACTTCTAATTCC |
| | | TGGATGGCCTGGTTCAGACAGGCCCCAGGCAAGGAACGCGAAGGCGTGGCTGCC |
| | | ATTTACACGGTCGGAGGGTCCATCTTTTACGCCGATTCCGTGCGTGGCCGCTTT |
| | | ACCATCTCCCAGGACGCTACTAAGAATATGTTCTACCTGCAAATGAATACCCTG |
| | | AAACCGGAGGACACCGCCATGTACTATTGTGCCGCAGCGTCCGGTCGCCTGAGG |
| | | GGGAAGTGGTTCTGGCCCTATGAATACAATTACTGGGGACAGGGCACCCAGGTT |
| | | ACCGTGTCTTCA |
| DR596- | 1212 | CAGGTACAGCTGCAAGAGAGCGGAGGCGGATTGGTGCAGCCCGGAGGCAGTTTG |
| hIL27 | | AGGTTGAGCTGTACCGCTTCCGGCCTCACTTTCGATGACTCCGTCATGGGGTGG |
| Ra_VH | | TTCCGCCAAGCACCTGGAAAGGGACGGGAAGCTGTCTCCTGCATCAGCAGTAGC |
| H9 | | GGAGCCAACGCATTCTACGCTGACAGCGTCAAAGGCAGGTTCACAATTAGCCGC |
| | | GATAATGCAAAGAACACTCTGTACCTGCAAATGAACTCTTTGAAGCCCGAGGAC |
| | | ACCGCGACGTATTACTGCAAGAGAGGACATGCCTGTGCCGGTTATTACCCCATC |
| | | CCCTACGATGACTATTGGGGACAGGGTACTCAGGTGACGGTGAGTTCCGGCGGT |
| | | AGCGGTGGCAGTGGAGGGAGCGGTCAGGTGCAGTTGCAGGAGAGCGGGGGGGGA |
| | | AGCGTGCAGTCCGGGGGGTCCCTGCGCCTTAGTTGCGCCGCTTCCGGCTTCACT |
| | | TATAGCACCTCTAACTCTTGGATGGCTTGGTTTCGCCAGGCCCCCGGCAAGGAG |
| | | AGAGAGGGCGTGGCTGCCATCTACACAGTGGGCGGAAGCATCTTTTACGCGGAT |
| | | AGCGTGAGAGGCCGTTTCACAATCAGCCAGGATGCAACAAAGAATATGTTCTAT |
| | | CTCCAGATGAACACACTGAAGCCCGAAGACACCGCTATGTATTACTGTGCTGCC |
| | | GCTTCAGGACGCTTGAGGGGCAAATGGTTTTGGCCCTATGAATACAACTACTGG |
| | | GGACAGGGCACCCAGGTGACTGTAAGCAGC |
| DR596- | 1213 | CAGGTGCAGTTGCAGGAAAGCGGCGGAGGCCTGGTGCAGCCTGGCGGTTCCCTG |
| hIL27 | | CGTCTTTCCTGTACTGCCTCAGGACTTACCTTCGATGACAGTGTGATGGGTTGG |
| Ra_VH | | TTCCGTCAGGCCCCTGGTAAAGGGCGGGAAGCAGTCAGTTGTATCTCCAGCTCC |
| H10 | | GGTGCAAACGCATTCTACGCCGACTCTGTCAAGGGACGTTTCACTATCAGTCGC |
| | | GATAACGCTAAGAATACCCTGTACCTCCAGATGAACAGCCTGAAGCCTGAGGAT |
| | | ACGGCTACCTATTACTGTAAACGGGGACACGCCTGTGCTGGCTACTATCCCATT |
| | | CCATACGACGATTATTGGGGCCAGGGAACTCAGGTGACAGTGTCAAGGGGCGGT |
| | | GGCTCACAGGTTCAGGTCCAGGAATCTGGGGGGGGCTCTGTCCAGGCAGGAGGC |
| | | AGCTTGCGCCTGTCCTGTAGGGCAAGTGGAAGCACTTACTCCAACTACTGCCTG |
| | | GGCTGGTTCCGCCAAATTACTGGTAAAGAGCGGGAAGGTGTCGCGGTCATTAAC |
| | | TGGGTCGGCGGTATGCTGTATTTTGCTGATTCTGTGAAGGGTCGGTTCACTGTG |
| | | TCCCAGGACCAGGCTAAGAACACCTTGTATCTGCAAATGAACAGCCTGAAGCCA |
| | | GAGGATACCGCGATGTATTACTGCGCAGCCGAGAGCGTCTCTAGCTTTAGCTGC |
| | | GGCGGTTGGCTGACCAGGCCGGATCGCGTGCCTTACTGGGGTCAAGGAACCCAG |
| | | GTGACCGTCTCTTCA |
| DR596- | 1214 | CAAGTCCAACTCCAGGAGTCCGGGGGTGGCCTCGTCCAGCCTGGTGGCTCCCTT |
| hIL27 | | AGGCTGAGCTGCACTGCATCTGGCCTGACTTTCGATGACTCCGTCATGGGCTGG |
| Ra_VH | | TTCCGCCAGGCTCCGGGAAAGGGACGCGAGGCCGTGTCTTGTATCAGCTCCAGT |
| H10 | | GGTGCCAACGCTTTTTACGCTGACTCTGTAAAAGGGCGGTTCACTATCAGCCGC |
| | | GACAACGCCAAAAACACCCTCTACTTGCAGATGAATAGCCTGAAGCCTGAGGAC |
| | | ACCGCCACATACTATTGTAAACGTGGCCACGCTTGCGCCGGTTATTACCCTATC |
| | | CCCTACGATGACTACTGGGGCCAGGGCACCCAGGTGACCGTGAGTTCCGGTGGC |
| | | AGCGGCGGTTCTGGCGGATCAGGCCAGGTCCAACTTCAGGAAAGCGGAGGGGGG |
| | | AGTGTTCAGGCTGGTGGCTCCCTGCGTCTGTCGTGCCGTGCTAGTGGCTCTACC |
| | | TATTCTAACTACTGCCTCGGCTGGTTCCGCCAAATCACGGGTAAGGAGCGTGAG |
| | | GGGGTTGCCGTCATAAATTGGGGGGCGGGGATGCTTTACTTCGCCGATAGTGTG |
| | | AAGGGGCGTTTTACTGTGTCCCAGGACCAGGCAAAGAATACCCTGTACCTCCAA |
| | | ATGAACTCTCTGAAGCCCGAAGACACAGCCATGTACTATTGTGCCGCAGAGAGC |
| | | GTGTCATCCTTCTCTTGTGGCGGATGGCTGACCCGCCCTGATCGCGTGCCCTAC |
| | | TGGGGTCAGGGCACCCAGGTGACCGTCTCATCG |
| DR596- | 1215 | CAGGTGCAGCTCCAGGAATCAGGGGGGGGCCTCGTGCAGCCAGGGGGCAGCCTG |
| hIL27 | | AGACTCTCTTGCACAGCCAGCGGACTCACCTTTGATGACAGCGTCATGGGCTGG |
| Ra_VH | | TTCAGGCAGGCTCCCGGCAAAGGAAGGGAGGCCGTCTCCTGCATCTCTTCCTCT |
| H11 | | GGTGCTAATGCCTTCTACGCTGACTCCGTCAAGGGCCGCTTTACTATCCCCCGC |
| | | GACAACGCAAAGAACACTCTGTACCTCCAGATGAACTCCCTGAAGCCTGAGGAC |
| | | ACCGCGACATATTACTGTAAGAGAGGCCACGCCTGTGCGGGCTATTACCCCATC |
| | | CCCTACGATGACTATTGGGGTCAGGGCACGCAAGTTACAGTCTCCAGCGGGGGA |
| | | GGTTCCCAGGTCCAGCTCCAGGAATCTGGGGGGGGCAGTGTCCAGGCCGGAGGC |
| | | TCCCTGAGACTGAGCTGCCGGGCCTCCGGTTCCACATACTCCAACTACTGCCTG |
| | | GGCTGGTTCCGCCAGTCCACAGGTAAAGAGAGAGAGGGCGTCGCAGTGATTAAC |
| | | TGGGTTGGCGGAATGGTGTACTTTGCTGACTCCGTTAAGGGCCGCTTCACCGTC |
| | | TCCCAGGATCACGCTAAGAACACTGTCACCTTGCAGATGAACTCATTGAAGCCC |
| | | GAAGATACCGCCATGTACTATTGTGCTGCCGAGAGCGTTAGTTCATTTTCTTGC |
| | | GGCGGTTGGGTGACGCGCCCAGGCCGCGTGCCGTACTGGGGCCAGGGAACGCAA |
| | | GTGACCGTGTCATCC |
| DR596- | 1216 | CAGGTACAGTTGCAGGAGTCCGGTGGGGGACTGGTCCAGCCCGGGGGATCACTG |
| hIL27 | | CGCCTGAGCTGCACCGCATCCGGGCTGACCTTCGACGATAGCGTTATGGGTTGG |
| Ra_VH | | TTCCGCCAGGCCCCAGGTAAGGGACGCGAAGCTGTCAGGTGCATTTCTTCCTCC |
| H11 | | GGGGCCAATGCCTTCTATGCCGATTCTGTGAAAGGAAGATTCACCATCAGCCGC |
| | | GACAATGCTAAGAATACTCTGTACCTCCAGATGAACAGCCTCAAGCCGGAGGAC |
| | | ACTGCCACATATTACTGCAAGAGAGGTCATGCTTGCGCAGGTTATTACCCGATT |
| | | CCCTACGACGATTATTGGGGCCAGGGGACCCAGGTTACGGTTTCTAGCGGAGGG |
| | | AGCGGAGGGTCCGGTGGCAGTGGACAAGTTCAGCTCCAGGAAAGTGGGGGGGGC |
| | | TCTGTCCAAGCCGGGGGCTCCCTGCGGGTCTCCTGCCGGGCTTCTGGCTCTACT |
| | | TATTCTAACTATTGCTTGGGTTGGTTCCGTCAATCCACTGGGAAGGAGAGAGAG |
| | | GGCGTGGCCGTTATAAATTGGGTTGGCGGGATGCTCTACTTCGCTGATTCCGTC |
| | | AAAGGAAGGTTCACAGTGTCCCAGGACCACGCTAAGAACACTGTGACTCTGCAA |
| | | ATGAACTCCCTGAAGCCAGAGGACACTGCTATGTATTACTGTGCCGCTGAGAGC |
| | | GTCTCATCCTTCTCCTGCGGCGGGTGGGTGACCCGTCCTGGCCGTGTGCCTTAC |
| | | TGGGGGCAGGGCACCCAGGTGACCGTCTCTAGT |
| DR596- | 1217 | CAGGTGCAACTCCAGGAGAGCGGAGGTGGACTTGTTCAGCCCGGCGGAAGCCTT |
| hIL27 | | CGCCTTTCATGCACTGCCAGCGGACTGACGTTCGATGACTCAGTCATGGGCTGG |
| Ra_VH | | TTCAGGCAGGCCCCCGGTAAGGGCCGCGAAGCCGTGTCTTGTATCTCTAGCTCT |
| H12 | | GGAGCAAACGCTTTCTACGCGGATTCCGTGAAGGGCCGTTTTACAATTAGCAGA |
| | | GACAACGCCAAGAATACTCTGTACTTGCAGATGAACTCTCTGAAGCCTGAAGAT |
| | | ACCGCTACCTATTACTGTAAGAGAGGCCATGCGTGTGCAGGGTATTACCCTATT |
| | | CCTTACGACGATTACTGGGGTCAAGGAACCCAGGTAACCGTGTCTTCAGGGGGG |
| | | GGTTCCCAGGTGCAGCTCCAGGAGTCCGGGGGAGGCAGTGTGCAAGCAGGCGAG |
| | | AGCTTGCGTCTGAGTTGCCGCGCTTCCGGTTCAACTTACTCTAACTACTGTCTT |
| | | GGATGGTTCCGCCAGATCACCGGCAAGGAGCGGGAGGGTGTGGCTGTCATCAAC |
| | | TGGGTTGGGGGTATGCTTTACTTCGCAGACTCTGTGAAGGGCCGCTTTACCGTA |
| | | TCACAGGACCAAGCAAAGAACACCGTGTACCTGGAGATGAACAGTCTGAAGCCC |
| | | GAAGACACCGCTATGTACTATTGTGCCACCGAATCCGTCTCTTCCTTCTCCTGA |
| | | GGAGGCTGGCTGACACGCCCCGACCGGGTGCCCTATTGGGGCCAGGGAACTCAG |
| | | GTCACAGTCTCTAGT |
| DR596- | 1218 | CAGGTCCAGCTCCAGGAGTCTGGGGCGGGGCTCGTGCAGCCCGGAGGCACTTTG |
| hIL27 | | AGGGTGAGCTGCACCGCGAGCGGCCTGACTTTCGACGATAGTGTGATGGGCTGG |
| Ra_VH | | TTCCGTCAAGCCCCTGGCAAAGGCCGCGAGGGGGTTAGCTGTATCTCCTCTTCC |
| H12 | | GGGGCCAATGCCTTCTATGCTGACAGCGTGAAGGGCCGCTTTACCATCAGCCGT |
| | | GACAACGCCAAGAACACTCTTTACCTTCAGATGAACTCCTTGAAGCCGGAGGAC |
| | | ACCGCGACATACTATTGTAAGCGCGGCCACGCTTGTGCAGGCTATTACCCCATC |
| | | CCCTATGACGATTATTGGGGACAGGGCACCCAGGTGACCGTCTCCTCTGGGGGC |
| | | AGCGGAGGCTCTGGTGGCTCCGGGCAGGTGCAGCTCCAGGAGTCCGGCGGTGGC |
| | | TCCGTGCAGGCTGGCGAACAGCCTGCGCCTGTCCGTCGTGCGTCCGGCAGTACA |
| | | TATAGCAACTACTGTCTCGGATGGTTCCGTCAAATTACCGGGAAGGAACGCGAG |
| | | GGAGTGGCCGTTATCAACTGGGCGGGAGGCATGTTGTATTTTGCCGACAGTGTG |
| | | AAAGGCAGGTTTACAGTGAGCCAGGACCAGGCCAAGAACACGGTGTATCTCGAA |
| | | ATGAACAGCCTGAAGCCCGAAGACACTGCAATGTATTACTGCGCCACCGAGTCT |
| | | GTTAGCTCCTTTTCATGTGGTGGGTGGGTGACACGCCCAGACCGCGTGCCATAC |
| | | TGGGGTCAGGGAACCCAGGTTACCGTGAGTAGT |
| DR596- | 1219 | CAGGTGCAGTTGCAGGAGTCCGGTGGGGGACTCGTACAGCCAGGAGGTAGCCTT |
| hIL27 | | CGCCTGAGTTGCACTGCCTCCGGTCTGACCTTCGATGACTCCGTTATGGGTTGG |
| Ra_VH | | TTCCGCCAAGCCCCAGGGAAGGGCAGAGAAGCTGTCTCTTGTATCAGCTCTAGC |
| H13 | | GGGGCCAACGCCTTCTACGCAGACTCCGTGAAGGGCCGCTTTACCATCAGTCGT |
| | | GACAACGCCAAGAATACACTCTATTTGCAAATGAACTCTCTCAAGCCTGAGGAT |
| | | ACCGGTACGTACTATTGCAAACGTGGACACGCTTGTGCCGGTTATTACCCTATC |
| | | CCTTACGATGACTACTGGGGCCAGGGCACCCAGGTGACCGTGAGTAGCGGCGGA |
| | | GGCTCTCAGGTGCAGCTCCAGGAGTCTGGTGGGGGGAGCGTCCAGGCCGGGGGG |
| | | TCTCTGCGCGTGTCCTGCGTGGCTTCCGGGTATGTTTCCTGCGATTACTTTCTG |
| | | CCCTCCTGGTATCGTCAGGCCCCTGGTAAGGAGCGCGAGTTCGTGAGCATCATT |
| | | GATGGCACTGGATCTACTTCTTACGCAGCGAGTGTGAAGGGCCGGTTCACCGCC |
| | | AGCCAGGATAGGGGAAAAAATATCGCATACCTCCAGATGAACAGCCTGAAGCCT |
| | | GAGGACACTGCCATGTATTACTGCAAGGCGTCCTGTGTGAGGGGAAGGACTATC |
| | | AGCGAGTATTGGGGCCAAGGTACGCAGGTGACAGTAAGCTCC |
| DR596- | 1220 | CAAGTTCAACTTCAGGAGAGTGGTGGAGGCCTGGTGCAGCCAGGGGGTTCCCTC |
| hIL27 | | AGGCTCAGCTGTACCGCAAGCGGTCTGACATTTGATGACTCCGTGATGGGATGG |
| Ra_VH | | TTTCGCCAGGCACCCGGTAAGGGGCGTGAAGCCGTTAGTTGTATTAGTTCCAGC |
| H13 | | GGAGCCAATGCTTTCTATGCCGACTCCGTGAAGGGTAGATTCACCATCCCCCGT |
| | | GACAACGCCAAAAACACGCTGTATCTTCAGATGAACTCTCTGAAGCCGGAGGAC |
| | | ACGGCCACCTATTACTGTAAGCGTGGTCATGCCTGCGCGGGGTATTACCCCATC |
| | | CCCTACGATGACTACTGGGGGCAGGGAACCCAGGTCACTGTTTCCAGCGGTGGC |
| | | TCCGGTGGCTCAGGAGGCAGTGGCCAGGTACAGTTGCAAGAGTCTGGCGGAGGC |
| | | AGCGTTCAGGCTGGGGGGTCCCTCAGACTGTCATGCGTGGCATCTGGGTATGTT |
| | | TCCTGCGACTACTTCCTTCCCAGTTGGTATCGTCAAGCGCCGGGTAAGGAACGC |
| | | GAATTTGTGAGTATCATTGATGGGACCGGGTCTACCTCTTACGCCGCGAGCGTG |
| | | AAGGGCCGCTTCACCGCGTCTCAGGATCGCGGTAAGAACATTGCCTACCTCCAG |
| | | ATGAACAGTCTGAAACCAGAAGACACCGCTATGTATTACTGCAAGGCGAGCTGG |
| | | GTGAGAGGCAGAACCATTTCTGAGTATTGGGGACAGGGAACTCAGGTGACCGTG |
| | | TCCTCA |
| DR596- | 1221 | CAGGTGCAGGTCCAGGAGTCAGGTGGCGGATTGGTGCAGCCCGGTGGCAGCTTG |
| hIL27 | | CGCCTGTCCTGCACAGCGAGTGGCTTGACTTTCGACGATTCCGTCATGGCGGGG |
| Ra_VH | | TTTAGACAGGCTCCTGGCAAGGGCCGTGAGGCAGTCTCGTGTATCTCTTCCAGT |
| H14 | | GGTGCTAACGCTTTTTACGCCGATAGTGTTAAGGGGAGATTCACGATCTCTCGC |
| | | GACAACGCCAAGAATACGCTTCTACTGCAGATGAACTCTCTCAAGCCTGAAGAT |
| | | ACCGCCACCTATTACTGCAAGCGTGGTCACGCTTGCGCAGGCTATTACCCCATC |
| | | CCCTACGACGATTACTGGGGCCAGGGCACCCAGGTGACCGTCAGTTCCGGTGGG |
| | | GGAAGCCAGGTCCAGCTTCAGGAGTCCGGGGGTGGCTCCGTCCAGGCAGGTGGC |
| | | TCCCTGCGCCTGTCTTGCGTGGCTTCTGGGTACGTGAGCTGCGACTACTTTCTC |
| | | CCCTCTTGGTATCGCCCAGGCCCCCGGAAAGGAGCGCGAGTTCGTTTCTACATT |
| | | GACGGCACTGGCTCCACCAGCTATGCTGCCAGCGTGAAAGGCCGCTTTACCGCC |
| | | AGCCAGGACAAAGGCAAGAACATCGCTTACCTCCAGATGAACCCCCTGAAGCCC |
| | | GAAGATACCGCGATGTATTACTGTAAGGCTTCATGCGTGCGCGGCAGAGCCATC |
| | | TCCGAGTACTGGGGCCAAGGCACTCAAGTTACAGTCTCTTCA |
| DR596- | 1222 | CAGGTGCAGTTGCAGGAGAGCGGTGGGGGCCTGGTGCAGCCCGGTGGCTCCCTG |
| hIL27 | | CGTCTTTCTTGCACTGCGTCCGGCTTGACTTTTGACGATAGCGTCATGGGCTGG |
| Ra_VH | | TTCCGCCAGGCCCCTGGCAAAGGACGTGAGGCCGTGAGCTGCATCTCTTCAAGT |
| H14 | | GGAGCCAACGCATTCTATGCTGACTCCGTGAAAGGCCGGTTCACCATCAGTCGG |
| | | GACAACGCCAAGAACACCCTCTACTTGCAGATGAACTCCCTGAAGCCCGAGGAC |
| | | ACCGCCACCTATTACTGTAAGCGCGGCCACGCTTGCGCTGGCTATTACCCGATC |
| | | CCATACGATGACTACTGGGGACAAGGCACCCAGGTTACCGTGTCTTCCGGGGGC |
| | | TCTGGTGGCTCAGGGGGCTCTGGCCAGGTCCAACTCCAAGAGTCTGGGGGAGGC |
| | | TCTGTGCAGGCGGGGGCTCCCTGCGTCTCGAGCTGCGTAGCTTCCGGTTACGTC |
| | | TCCTGCGATTACTTCGTGCCCTCCTGGTATAGGCAGGGTCCCGGCAAGGAGCGC |
| | | GAGTTTGTCTCTATTATCGACGGTACAGGCTTCTACCTCTACGCCGCAAGCGTG |
| | | AAGGGCCGGTTCACAGCCTCTCAGGATAAGGGTAAAAACATCGCCTACTTGCAG |
| | | ATGAACAGCGTTAAACCAGAGGACACCGCCATGTACTATTGTAAGGCAAGCTGC |
| | | GTGAGGGGCCGCGCCATCAGCGAGTACTGGGGCCAGGGAACGCAGGTGACCGTC |
| | | AGCAGC |
| DR596- | 1223 | CAGGTCCAGCTCCAGGAGTCCGGGGGTGGCCTGGTCCAGCCCGGGGGGTCTCTG |
| hIL27 | | AGACTGTCCTGTACGGCAAGCGGGCTGACCTTCGATGACTCCGTCATGGGCTGG |
| Ra_VH | | TTTCGTCAAGCCCCTGGTAAGGGCCGCGAGGCGGTTTCTTGCATCAGCTCTTCA |
| H15 | | GGCGCGAATGCGTTCTACGCCGACTCTGTGAAGGGTCGGTTTACTATCAGCCGC |
| | | GATAATGCGAAGAACACACTGTACTTGCAAATGAACTCCCTGAAGCCTGAGGAC |
| | | ACAGCCACTTATTACTGCAAACGTGGACACGCCTGCGCTGGCTATTACCCAATC |
| | | CCTTACGACGATTACTGGGGCCAGGGCACCCAAGTGACCGTGAGTTCCGGCGGT |
| | | GGCTCCCAAGTCCAGGTCCAGGAAAGTGGTGGGGGGTCCGTGCAGGGGGGAGGC |
| | | AGCCTTAGGCTCTCTTGCGTCGCCTCTGGGTACGTCTCTTGCGACTATTTCCTC |
| | | CCTTCCTGGTATCGCCAAGCGCCGGGCAAGGAGCGCGAGTTCGTCTCCATCATT |
| | | GATGGCACCGGCTCCACCTCCTACGCTGCCTCCGTGAAAGGCCGGTTCACTGCC |
| | | TCCCAGGACAAGGGGAAAAACATCGCCTACTTGCAGATGAACACCCTTAAACCC |
| | | GAAGACACGGCCATGTATTACTGTAAAGCGTCCTGCGTGAGGGGAAGAGCAATC |
| | | TCCGAGTACTGGGGCCAGGGGACCCAGGTGACAGTGTCGCA |
| DR596- | 1224 | CAGGTGCAGTTGCAGGAGTCCGGTGGCGGACTGGTACAGCCCGGCGGTTCCTTG |
| hIL27 | | AGGCTGAGTTGTACCGCTTCCGGCTTGACTTTCGATGACTCAGTGATGGGATGG |
| Ra_VH | | TTCAGACAAGCGCCCGGTAAGGGTCGCGAGGCGGTCAGTTGCATCAGCTCCAGT |
| H15 | | GGAGCCAATGCGTTCTACGCTGATTCTGTGAAAGGACGTTTCACCATCTCCAGG |
| | | GACAATGCTAAGAATACACTGTACCTTCAGATGAACAGCCTGAAGCCTGAAGAT |
| | | ACAGCTACGTATTACTGCAAGCGCGGACACGCTTGTGCAGGATATTACCCAATT |
| | | CCTTATGATGACTACTGGGGTCAGGGGACCCAGGTGACTGTATCCAGCGGGGGC |
| | | TCCGGCGGAAGCGGAGGCAGTGGGCAGGTGCAGCTCCAGGAGTCCGGCGGAGGC |
| | | TCAGTTCAGGCCGGAGGTTCACTCAGACTGTCATGTGTGGCCTCTGGCTACGTG |
| | | TCCTGTGACTACTTCCTGCCAAGTTGGTATCGGCAGGCCCCTGGCAAGGAGCGG |
| | | GAGTTCGTTAGCATTATCGACGGCACGGGCAGCACCAGTTACGCTGCCTCCGTG |
| | | AAAGGCAGATTCACAGCATCTCAGGATAAGGGGAAAAATATCGCCTACCTCCAG |
| | | ATGAACACTCTGAAGCCCGAGGACACCGCCATGTACTATTGTAAGGCTTCATGC |
| | | GTGAGAGGCCGCGCCATTAGCGAGTACTGGGGCCAGGGAACCCAGGTTACTGTC |
| | | AGTTCT |
| DR596- | 1225 | CAGGTGCAGTTGCAGGAATCTGGCGGTGGACTGGTGCAGCCCGGAGGCTCACTG |
| hIL27 | | CGCCTCTCTTGTACGGCCAGCGGACTCACTTTCGATGACAGCGTGATGGCGGGG |
| Ra_VH | | TTCCGCCAGGCCCCTGGCAAAGGCCGCGAGGCTGTCTCCTGTATCTCCAGCAGT |
| H16 | | GGTGCGAACGCCTTCTACGCTGACTCCGTGAAAGGGCGTTTCACCATCAGCCGT |
| | | GATAACGCTAAGAACACACTCTACTTGCAGATGAACAGCCTGAAGCCGGAGGAC |
| | | ACTGCCACCTATTACTGTAAGCGCGGTCACGCATGTGCTGGCTATTACCGTATT |
| | | CCTTACGACGATTACTGGGGCCAGGGCACCCAGGTTACGGTGTCCTCTGGGGGG |
| | | GGTAGCCAGGTGCAGCTTCAGGAGTCCGGTGGAGGTTCCGTGCAGGCAGGGGGA |
| | | AGCCTTAGGGTGAGCTGCCGCGCTTCCGGTTCTACCTACAGCAATTACTGCCTG |
| | | GGGTGGTTCCGGCAGATTACAGGTAAAGAGCGCGAGGGTGTCGCCGTGATTAAC |
| | | TGGGTGGGGGGATGCTTTACTTCGCTGATTCAGTGAAGGGCCCGCTTCACTGTG |
| | | AGTCAGGACCAGGCCAAGAACACAGTGTACCTCCAGATGAACTCCCTTAAACCC |
| | | GAGGACACTGCAATGTACTATTGCGCTGCCGAGTCAGCGTCCTCTTTCAGTTGC |
| | | GGCGGATGGCTCACTCGTCCTGACCGTGTCCCTTACTGGGGCCAGGGCACGCAG |
| | | GTGACCGTCTCCTCC |
| DR596- | 1226 | CAGGTGCAGTTGCAGGAGAGGGGCGGAGGCCTGGTCCAGCCCGGAGGCAGCCTG |
| hIL27 | | AGACTTTCTTGCACAGCTTCCGGGCTTACCTTCGATGACTCCGTCATGGGCTGG |
| Ra_VH | | TTTCGGCAGGCTCCAGGTAAGGGTAGGGAGGCTGTTAGCTGTATCAGTTCCAGC |
| H16 | | GGCGCTAATGCCTTCTACGCCGACTCTGTGAAGGGCCGGTTTACCATCAGCAGA |
| | | GACAACGCAAAAAATACGCTCTACTTGCAGATGAACTCTCTGAAGCCCGAAGAT |
| | | ACCGCCACCTACTATTGCAAGAGGGGCCACGCCTGCGCCGGTTACTATCCAATC |
| | | CCATACGATGACTACTGGGGCCAGGGTACTTCAGGTGACTGTGTCTCCGGTGGA |
| | | AGCGGCGGAAGCGGCGGTAGGGGGCAGGTCCAGCTGCAAGAAAGCGGAGGCGGG |
| | | TCCGTGCAAGCTGGGGGTTCCCTGAGGCTGAGCTGTCGTGCATCAGGCTCAACC |
| | | TACTCTAACTACTGCTTGGGGGGTTCCGCCAGATTACCGGCAAGCGAGCGCGAG |
| | | GGCGTGGCCGTCATCAACTGGGTAGGCGGGATGCTGTATTTCGCTGATTCCGTG |
| | | AAGGGCCGCTTTACCGTCTCACAGGACCAGGCCAAGAACACCGTCTACCTTCAG |
| | | ATGAACTCCCTGAAACCTGAAGATACTGCCATGTATTACTGTGCAGCGGAGAGC |
| | | GCCAGCTCCTTCTCCTGTGGAGGGGGCTGACCCGCCCGGATCCGTGTTCCGTAC |
| | | TGGGGCCAGGGGACTCAAGTGACCGTGTCCTCC |
| DR596- | 1227 | CAGGTGCAGGTCCAGGAATCAGGGGGGGGACTGGTTCAGCCCGGAGGTTCTCTC |
| hIL27 | | AGGCTCAGCTGCACTGCCTCCGGGCTTACTTTTGACGATAGCGTTATGGGCTGG |
| Ra_VH | | TTCCGCCAGGCTCCCGGCAAAGGCCGCGAGGCCGTGTCTTGTATCTCCAGCTCC |
| H17 | | GGTGCCAACGCCTTCTACGCTGACTCCGTGAAGGGACGCTTTACCATCTCACGC |
| | | GACAACGCGAAGAACACACCTTACCTGCAAATGAACAGCTTGAAACCTGAGGAT |
| | | ACAGCAACCTACTATTGCAAGCGCGGTCACGGTTGCGCCGGTTATTACCCTATC |
| | | CCTTACGATGACTACTGGGGTCAGGGTACTCAGGTAACGGTGTCAAGTGGGGGC |
| | | GGGTCCCAGGTCCAGCTTCAAGAGTCCGGCGGGGGACTGGTGCAGCCCGGTGGC |
| | | TCTCTGAGGCTCTCTTGTGCTGCGAGCGGATTCACTTTGTCTCTGTCAGGGATG |
| | | TCTTGGGTGCGTCAAGCACCTGGGAAGGGCCTGGAATGGGTCTCAGCAATCTCC |
| | | AGCGGCGGTGCTTCCACCTATTACACTGACAGCGTGAAGGGCAGGTTTACCATC |
| | | TCCAGAGACAACGCCAAGAACATGCTGTATCTCCAGTTGAACTCACTGAAAACC |
| | | GAGGATACAGCCATGTATTACTGTGCCAAAGGAGGGAGCGGCTACGGTGATGCC |
| | | TCTCGTATGACCAGCCCCGGCTCCCAGGGAACCCAGGTGACGGTGTCTTCC |
| DR596- | 1228 | CAGGTCCAGCTCCAGGAGTCTGGGGGGGGCCTGGTCCAGCCTGGGGGTAGCCTG |
| hIL27 | | AGACTGTCCTGCACTGCTTCTGGTCTTACCCTTGATGACAGCGTGATGGGCTGG |
| Ra_VH | | TTTAGACAAGCGCCCGGCAAAGGCCGCGAAGCCGTGAGTTGTATCTCCTCTAGC |
| H17 | | GGCGCGAACGCATTCTACGCTGACTCTGTCAAGGGCCGGTTCACCATTTCCAGG |
| | | GATAACGCCAAGAACACCCTGTACCTTCAGATGAACAGCCTGAAACCCGAAGAC |
| | | ACCGCCACTTATTACTGCAAGCGGGGCCACGCCTGTGCTGGCTATTACCCTATT |
| | | CCTTACGACGATTACTGGGGGCAGGGCACCCAGGTAACTGTCTCTTCAGGCGGA |
| | | AGCGGCGGTTCCGGGGGGTCTGGGCAGGTGCAGTTGCAAGAGTCCGGGGCGGGG |
| | | CTGGTCCAGCCGGGTGGCTCACTCCGCTTGTCCTGCGCGGCTTCAGGATTTACT |
| | | TTCAGTCTGAGTGGTATGAGCTGGGTACGCCAGGCACCCGGCAAGGGCCTGGAG |
| | | TGGGTGTCCGCCATTTCCTCTGGGGGAGCGAGCACATACTATACCGATTCAGTG |
| | | AAAGGGCGTTTTACTATCTCTCGCGATAATGCCAAGAACATGCTGTACCTCCAG |
| | | CTCAACTCTCTGAAGACTGAGGACACCGCCATGTACTATTGGGCCAAAGGCGGA |
| | | AGTGGATACGGTGACGCCTCAAGGATGACTTCCCCTGGTTCCCAGGGCACGCAG |
| | | GTGACCGTGAGTAGG |
| DR596- | 1229 | CAGGTGCAGCTCCAGGAATCCGGTGGGGGACTCGTCCAGCCAGGGGGGTCACTG |
| hIL27 | | CGTCTCTCCTGTACCGCGAGTGGACTGACATTCGATGACTCCGTGATGCGGGGG |
| Ra_VH | | TTCCGCCAGGCCCCCGGCAAGGGCCGCGAGGCGGTGTCTTGCATTTCTAGCTCT |
| H18 | | GGGGCCAATGCGTTTTACGCTGACTCTGTGAAGGGCAGATTCACTATCAGCAGA |
| | | GACAACGCGAAGAACACGCTGTATCTGCAAATGAACTCTCTGAAACCCGAGGAT |
| | | ACTGCCACATACTATTGTAAGCGCGGCCACGCGTGCGCAGGCTACTATCCTATT |
| | | CCTTACGATGACTACTGGGGCCAGGGAACACAGGTCACCGTTAGCTCAGGGGGA |
| | | GGCTCCCAGGTGCAGCTCCAGGAGAGCGGAGGTGGCAGCGTCCAGGCTGGCGGA |
| | | TCTCTGCGCCTGTCTTGTGTCGCCAGCGGATACGTGAGGTGTGACTACTTCCTG |
| | | CCTAGCTGGTATCGTCAAGCGCCGGGGAAGGAAAGGGAGTTCGTGTCCATTATC |
| | | GACGGTACTGGGTCTACCTCATACGCGGCCCCCGTCAAGGGCCGTTTCACGGCC |
| | | TCCCAGGACAAGGGGAAAAACATCGCATATCTGCAAATGAACTCTCTGAAGCCT |
| | | GAGGACACCGCAATGTATTACTGCAAGGCCTCCTGTGTACGCGGACGTGGAATC |
| | | TCTGAGTATTGGGGCCAGGGGACCCAGGTGACCGTGTCTTCC |
| DR596- | 1230 | CAGGTGCAGCTCCAGGAATCCGGGGGGGGTCTGGTTCAGCCAGGTGGCTCTCTC |
| hIL27 | | CGTCTGAGCTGCACCGGTTCCGGTCTCACATTCGACGATTCCGTAATGGGGTGG |
| Ra_VH | | TTCCGCCAGGCCCCTGGAAAAGGTCGCGAGGCCGTGTCTTGCATTTCTTCCAGC |
| H18 | | GGGGCGAACGCATTCTACGCAGACTCTGTGAAGGGTCGTTTTACAATCTCCCGG |
| | | GACAACGCGAAGAACACCCTTTACTTGCAAATGAACTCCCTCAAGCCCGAGGAC |
| | | ACCGCGACCTACTATTGCAAGCGTGGCCATGCTTGCGCCGGGTATTACCCCATC |
| | | CCCTATGATGACTACTGGGGACAGGGAACCCAAGTGACAGTTTCTAGCGGAGGC |
| | | AGTGGTGGCAGCGGTGGGTCCGGCCAGGTGCAGTTGCAAGAGAGTGGGGGAGGC |
| | | TCTGTCCAAGCTGGCGGTTCCCTCCGCCTGTCCTGCGTGGCCTCTGGCTACGTT |
| | | AGCTGCGACTATTTCCTGCCTTCATGGTATCGTCAGGCCCCAGGGAAAGAAAGG |
| | | GAGTTCGTTAGCATCATTGATGGAACTGGCTCCACCTCCTATGCTGCAAGCGTG |
| | | AAGGGTCGGTTCACCGCCTCCCAGGATAAGGGCAAGAATATCGCTTATTTGCAG |
| | | ATGAACTCTCTGAAGCCAGAGGATACCGCCATGTACTATTGCAAGGCATCCTGC |
| | | GTGGGGGGACGCGGGATCAGCGAATATTGGGGCCAGGGAACTCAGGTGACCGTC |
| | | AGTTCT |
| DR596- | 1231 | CAGGTTCAGCTCCAGGAAAGCGGAGGCGGTCTCGTACAGCCTGGCGGTTCCCTG |
| hIL27 | | AGGCTGTCCTGCACCGCCAGCGGACTCACGTTTGACGATAGTGTAATGGGATGG |
| Ra_VH | | TTCAGGCAGGGTCCCGGCAAGGGCCGCGAGGCCGTAAGTTGCATGTCCAGCTCA |
| H19 | | GGTGCCAATGCGTTCTATGCCGACTCCGTGAAGGGTAGATTCACCATCAGCCGT |
| | | GATAACGCGAAAAACACGCTCTACCTCCAGATGAACTCACTGAAGCCTGAGGAC |
| | | ACGGCCACATATTACTGCAAGAGGGGCCACGCCTGTGGTGGGTATTACCCCATC |
| | | CCCTATGATGACTACTGGGGCCAGGGGACACAAGTGACCGTCTCCAGCGGGGGG |
| | | GGAAGCCAGGTGCAGTTGCAGGAATCCGGGGGTGGCAGCGTGCAGGCTGGTGGG |
| | | TCTCTCAGGGTCAGTTGTAGGGCCTCTGGTTCCACCTACAGCAACTACTGTCTG |
| | | GGCTGGTTCCGTCAGATCACTGGTAAAGAGAGGGAGGGCGTGGCAGTCATCAAC |
| | | TGGGTTGGCGGAATGTTGTATTTCGCTGACTCTGTCAAGGGCAGATTCACCGTC |
| | | TCCCAGGATCAGGCTAAGAACACAGTATACTTGCAGATGAACTCCCTGAAGCCC |
| | | GAGGATACTGCTATGTATTACTGTGCCGCAGAAAGCGTGTCCTCTTTCTCTTGC |
| | | GGTGGCTGGCTGACACGCCCTGACAGAGTCCCTTATTGGGGTCAGGGGACCCAG |
| | | GTAACGGTATCTTCC |
| DR596- | 1232 | CAGGTCCAGGTGCAAGAAAGCGGCGGAGGCCTGGTGCAGCCTGGGGGTTCTCTC |
| hIL27 | | CGTCTGAGCTGTACCGCCTCTGGCCTGACATTCGATGACTCCGTTATGGGTTGG |
| Ra_VH | | TTCCGCCAAGCGCCTGGGAAAGGAAGGGAGGCAGTGAGCTGTATTTCTAGCTCT |
| H19 | | GGAGCCAATGCTTTCTATGCTGATAGCGTGAAGGGGCGGTTCACTATCTCAAGG |
| | | GACAACGCCAAGAATACACTGTATCTTCAGATGAACAGCCTGAAGCCAGAGGAT |
| | | ACAGCCACCTATTACTGTAAGCGCGGACATGCCTGTGCTGGCTATTACCCGATT |
| | | CCCTACGACGATTACTGGGGGCAAGGAACTCAGGTTACTGTGAGTTCCGGGGGC |
| | | TCTGGGGGCTCAGGCGGTTCCGGCCAGGTACAACTCCAGGAGAGCGGCGGAGGC |
| | | TCCGTACAGGCCGGGGGCAGCGTGAGACTGAGGTGTCGCGCGAGCGGCAGTACC |
| | | TACAGCAATTACTGCCTGGGGGGTTCCGCCAGATTACTGGGAAGGAGCGGGGAA |
| | | GGCGTCGCAGTGATAAATTGGGTCGGTGGAATGCTCTACTTCGCCGATAGCGTC |
| | | AAGGGACGCTTCACCGTGAGTCAGGATCAGGCCAAAAACACCGTTTATCTTCAG |
| | | ATGAACTCCTTGAAGCCGGAAGACACAGCAATGTACTATTGCGCCGCTGAAAGT |
| | | GTCTCCAGCTTCAGCTGCGGAGGGTGGCTCACCCGCCCGGATCGCGTGCCTTAC |
| | | TGGGGGCAGGGCACCCAGGTCACTGTGTCCTCT |
| DR596- | 1233 | CAGGTGCAGTTGCAGGAGTCCGGGGGAGGCCTGGTGCAGCCTGGGGGCTCCCTG |
| hIL27 | | CGCCTCTCTTGTACTGCGTCAGGTCTGACTTTTGACGATTCTGTTATGGGATGG |
| Ra_VH | | TTCCGGCAAGCGCCCGGCAAGGGCCGCGAGGCGGTGAGTTGCATCTCCAGCTCT |
| H20 | | GGCGCAAACGCTTTCTATGCTGACAGTGTGAAGGGCCGTTTTACAATTTCCAGG |
| | | GACAATGCCAAGAATACACTGTACCTCCAGATGAACAGTCTGAAACCCGAAGAT |
| | | ACCGCAACCTATTACTGCAAGCGCGGCCACGCTTGCGCCGGATACTATCCAATC |
| | | CCCTATGATGACTACTGGGGCCAGGGCACGCAGGTCACCGTGAGTAGCGGGGGT |
| | | GGCTCCCAGGTTCAGCTTCAGGAGTCAGGTGGAGGCCTGGTTCAGCCAGGTGGC |
| | | AGCCTGCGCTTGAGCTGCGCGGCCAGCGGTTTCACCCTTAGCTGTTACCCGATG |
| | | TCTTGGGTCAGACAGGCTCCCGGCAAGGGCCTGGAGTGGGTGTCTACCATCTCT |
| | | TCCGGGGGCGATACCACTCTGTACGCGGATTCCGTGAAAGGTCGCTTTACCTCC |
| | | TCTAGGGATAATGCTAAAAATACGCTGTACCTCCAGCTTAATTCCCTGAAGACA |
| | | GAAGATACCGCCATGTATTACTGCGCGAAGCGCATCGACTGCAACTCCGGGTAC |
| | | TGTTACAAACGCAGCTATTGGGGCCAGGGAACACAGGTGACCGTGTCCCCC |
| DR596- | 1234 | CAGGTGCAACTTCAGGAATCCGGCGGGGGCTTGGTGCAGCCAGGGGGGTCTCTC |
| hIL27 | | CGCCTGTCCTGCACGGCCTCTGGCCTGACCTTTGACGATTCCGTGATGGGGTGG |
| Ra_VH | | TTTCGCCAAGCGCCAGGCAAGGGGGGGGAGGCCGTTAGTTGCATCAGTTCCTCC |
| H20 | | GGAGCCAACGCTTTCTACGCCGACTCCGTCAAGGGCCGTTTCACCATCAGCAGA |
| | | GACAACGCCAAGAATACACTGTATCTCCAGATGAACAGCCTCAAACCGGAGGAC |
| | | ACGGCCACCTACTATTGTAAGCGCGGTCACGCATGTGCGGGGTATTACCCAATC |
| | | CCGTATGATGACTACTGGGGTCAGGGCACCCAGGTCACCGTTAGCTCCGGTGGA |
| | | TCTGGCGGAAGCGGGGGCAGCGGACAGGTCCAGCTCCAGGAGAGCGGGGGGGGA |
| | | CTGGTTCAGCCTGGGGGGTCTCTGCGTCTGTCCTGCGCCGCTTCAGGCTTCACC |
| | | TTTTCATCTTATCCGATGTCCTGGGTCAGACAAGCCCCAGGTAAGGGGCTGGAG |
| | | TGGGTTTCTACGATTAGCTCTGGTGGCGATACCACACTGTACGCGGACAGCGTG |
| | | AAGGGGCGGTTCACCAGCTCAAGAGACAACGCCAAAAACACCTTGTACCTCCAG |
| | | CTGAACTCCCTGAAGACCGAGGACACCGCCATGTATTACTGCGCCAAGAGAATT |
| | | GACTGTAACAGCGGTTATTGTTATAAAAGATCCTACTGGGGCCAGGGAACCCAG |
| | | GTGACAGTCAGTTCT |
| DR596- | 1235 | CAGGTGCAGTTGCAGGAGAGCGGTGGAGGCCTGGTCCAGCCAGGTGGCTCTCTG |
| hIL27 | | CGGCTGAGCTGCACCGCTTCTGGTCTGACCTTTGATGACAGCGTGATGGCGGGG |
| Ra_VH | | TTCCGCCAGGCTCCCGGAAAGGGGCGCGAAGCTGTGTCCTGCATCAGCTCCTCC |
| H21 | | GGTGCCAACGCCTTTTACGCTGATTCCGTGAAGGGTCGCTTCACCATTAGCCGC |
| | | GACAACGCAAAGAATACCCTCTACTTGCAGATGAACAGCTTGAAGCCCGAGGAC |
| | | ACAGCGACCTATTACTGTAAGCGCGGCCATGCGTGCGCAGGCTACTATCCGATC |
| | | CCCTATGATGACTACTGGGGCCAGGGAACCCAGGTGACTGTCTCTTCCGGTGGC |
| | | GGTTCCCAGGTCCAGCTTCAGGAGAGCGGTGGCGGACTGGTTCAGCCCGGGGGG |
| | | AGCCTCAGGTTGAGCTGTGCCGCTAGTGGCTTCACCTTCTCCCTCTCTAGCATG |
| | | AGCTGGGTTCGTCAGGCCCCAGGCAAAGGGCTCGAATGGGTCAGCGCAATCTCC |
| | | AGCGGGGGTGCCTCCACCTATTACACAGACTCTGTGAAAGGAAGATTCACTATC |
| | | TCACGTGACAATGCTAAAAACATGCTGTACCTTCAGCTGAACAGCCTGAAGACC |
| | | GAGGATACCGCTATGTACTATTGTGCCAAGGGTGGCTCCGGCTATGGCGACGCA |
| | | TCACGCATGACCTCTCCGGGCTCCCAAGGAACCCAAGTTACCGTATCTTCA |
| DR596- | 1236 | CAGGTGCAGCTCCAGGAGTCCGGTGGAGGGCTGGTACAACCCGGCGGTTCCCTG |
| hIL27 | | CGCCTGTCCTGCACAGCAAGCGGGCTTACTTTTGATGACAGTGTCATGGGATGG |
| Ra_VH | | TTCCGCCAAGCGCCCGGTAAGGGCAGGGAGGCTGTCAGCTGTATCAGTTCCAGC |
| H21 | | GGGGCTAATGCGTTTTATGCCGATAGTGTCAAGGGCCGGTTTACCATCAGCCGC |
| | | GACAACGCCAAAAACACCCTGTATTTGCAGATGAATAGCCTCAAACCAGAGGAC |
| | | ACAGCGACCTATTACTGTAAGCGCGGTCATGCGTGCGGGGGCTATTACCCGATT |
| | | CCTTACGACGATTATTGGGGACAGGGCACCCAGGTGACCGTCAGCTCCGGTGGC |
| | | AGTGGCGGTTCCGGTGGCTCCGGCCAGGTGCAGCTTCAGGAGAGCGGTGGGGGG |
| | | CTGGTGCAGCCCGGAGGTTCCCTCCGCCTGTCGTGTGCTGCCAGGGGCTTCACA |
| | | TTTTCCCTGTCATCCATGAGCTGGGTGCGCCAGGCACCCGGTAAGGGACTGGAA |
| | | TGGGTTTCTGCGATTTCTTCCGGCGGTGCCTCCACCTACTATACCGATAGCGTC |
| | | AAGGGGCGGTTTACGATTTCCAGAGACAACGCCAAGAATATGCTGTACCTTCAG |
| | | CTCAATTCTCTTAAAACCGAGGACACCGCCATGTATTACTGTGCTAAAGGGGGC |
| | | TCTGGATATGGTGATGCCTCACGCATGACATCCCCTGGATCTCAGGGCACCCAG |
| | | GTAACCGTGTCCTCA |
| DR596- | 1237 | CAGGTCCAACTCCAGGAGAGGGGGGAGGCTTGGTGCAGCCCGGGCGGGTCCCTG |
| hIL27 | | CGGCTGTCTTGCACTGCTAGTGGACTGACCTTCGACGATTCCGTGATGCGGGGG |
| Ra_VH | | TTCCGCCAAGCTCCGGGAAAGGGCCGCGAGGCCGTGAGTTGCATTTCTTCCAGT |
| H22 | | GGTGCAAACGCCTTTTATGCGGACAGCGTTAAAGGACGGTTCACCATCTCACGC |
| | | GATAACGCGAAGAATACACTGTATCTTCAGATGAACTCCCTGAAGCCCGAGGAT |
| | | ACCGCTACCTACTATTGTAAGAGGGGTCATGCGTGTGCGGGATACTATCCAATC |
| | | CCGTATGACGATTATTGGGGTCAAGGCACTCAGGTTACTGTGTCTTCCGGTGGA |
| | | GGGTCCCAGGTGCAGTTGCAGGAGTCAGGAGGGGGTAGCGTGCAGGCCGGAGGG |
| | | AGCCTGCGCCTGTCTTGTCGCGCATCCGGGAGCACATATTCAAATTATTGTCTC |
| | | GGGGGTTCCGCCAAACCACAGGCAAGGAACGGGAGGGCCGTGGCTGTAATCAAC |
| | | TGGGTCGGTGGAATGGTCTACTTTGCTGACTCTGTGAAAGGCCGCTTCACTGTC |
| | | TCCCAGGACCAGGCCAAGAACACCGTTTATCTTCAGATGAACTCCCTGAAGCCC |
| | | GAAGACACCGCCATGTATTACTGCGCTGCCGAGAGCGTCTCCAGGTTCTCCTGC |
| | | GGCGGATGGCTGACCCGTCCTGATAGAGTGCCCTATTGGGGCCAAGGCACCCAG |
| | | GTTACCGTGTCCAGC |
| DR596- | 1238 | CAGGTGCAGCTCCAGGAGAGTGGGGGGGGCTTGGTGCAGCCAGGCGGATCTCTG |
| hIL27 | | AGGCTGTCTTGCACAGCGAGCGGCCTGACATTCGATGACTCCGTGATGGGCTGG |
| Ra_VH | | TTTCGCCAGGCCCCCGGCAAGGGCCCGCGAGGCCGTCTCGGTATCAGTTCATCT |
| H22 | | GGAGCAAACGCCTTTTACGCTGATTCCGTGAAGGGACGCTTTACCATTAGCCGG |
| | | GACAACGCCAAGAACACCCTCTACCTCCAGATGAACAGCCTGAAGCCTGAAGAT |
| | | ACCGCAACGTATTACTGTAAGAGAGGCCATGGTTGTGCGGGTTACTATCCAATC |
| | | CCCTATGACGATTACTGGGGTCAGGGCACCCAAGTCACCGTGTCTAGCGGAGGG |
| | | TCTGGAGGCTCCGGGGGTAGCGGTCAGGTGCAGCTCCAGGAATCTGGTGGCGGA |
| | | AGCGTGCAGGCGGGGGGTAGCCTGCGCGTGAGCTGTCGCGCCAGCGGTAGCACC |
| | | TACAGCAATTATTGTCTCGGCTGGTTTCGTCAGACCACTGGCAAGGAACGGGAA |
| | | GGCGTTGCCGTTATCAACTGCGGGCGGGGTATGCTGTACTTCGCCGATAGTGTG |
| | | AAGGGTCGCTTCACCGTGTCCCAGGACCAGGCTAAGAACACTGTGTATCTCCAG |
| | | ATGAACTCCTTGAAGCCCGAGGACACCGCGATGTACTATTGCGCCGCTGAGTCT |
| | | GTCAGCTCTTTTTCCTGCGGCGGGTGGGTGACGCGGCCCGACCGGGTGCCATAT |
| | | TGGGGCCAAGGTACTCAGGTGACCGTTAGCAGT |
| DR596- | 1239 | CAGGTGCAGCTTCAGGAATCCGGGGGAGGCCTGGTGCAACCTGGAGGTAGCTTG |
| hIL27 | | CGTCTGTCATGCACAGCCAGTGGACTTACTTTCGACGATAGCGTGATGGGTTGG |
| Ra_VH | | TTCCGGCAGGCTCCTGGCAAGGGCCGTGAGGCCGTGAGCTGCATCTCCAGCTCC |
| H23 | | GGTGCCAATGCCTTTTACGCTGATTCCGTCAAAGGACGGTTCACTATCTCCCGC |
| | | GATAACGCCAAAAACACATTGTATCCCCAGATGAATAGTCTGAAGCCGGAGGAT |
| | | ACCGCAACCTATTACTGCAAGCGCGGCCACGCTTGCGCAGGATACTATCCTATC |
| | | CCTTATGACGATTATTGGGGACAGGGAACACAAGTGACGGTGTCCTCTGGCGGA |
| | | GGTTCCCAGGTGCAGTTGCAGGAGTCTGGGGGGGGGTCCGTCCAGGCCGGAGGC |
| | | AGCCTGCGCCTTTCCTGTCGCGCCAGCCGCTCCCCCTACGGGAACTATTGTCTG |
| | | GGCTGGTTTCGCCAGTCCACTGGTAAGGAAAGAGAGGGGGTAGCCGTGATAAAT |
| | | TGGGTCGGGGGTATGTTGTACTTCGCTGACAGCGTTAAGGGAAGATTCACCGTG |
| | | AGCCAGGATCACGCGAAGAACACCGTCACCCTCCAGATGAACTCTCTCAAGCCC |
| | | GAGGATACAGCTATGTATTACTGTGCCGCTGAGTCCGTTAGCTCCTTTTCATGG |
| | | GGCGGATGGCTGACCCGCCCCGACCGTGTCCCCTATTGGGGCCAGGGCACCCAA |
| | | GTTACTGTGAGTTCT |
| DR596- | 1240 | CAAGTGCAGCTCCAGGAGTCTGGGGGGGGTCTTGTTCAGCCAGGAGGCTCTTTG |
| hIL27 | | CGGCTGTCCTGTACCGCTTCCGGCCTGACATTCGATGACTCTGTCATGGGCGGG |
| Ra_VH | | TTCAGACAAGCCCCTGGGAAGGGACGCGAAGCCGTGAGCTGCATCTCTAGCTCT |
| H23 | | GGGGCTAACGCCTTTTACGCGGACTCCGTGAAAGGAAGATTCACCATCTCCAGA |
| | | GACAACGCCAAGAACACACTCTACCCCCAGATGAACTCCCTGAAACCCGAGGAC |
| | | ACCGCCACCTATTACTGCAAGCGCGGCCATGCGTGCGGGGGCTATTACCCTATT |
| | | CCTTATGATGACTACTGGGGGCAGGGGACACAGGTGACCGTTTCTAGCGGCGGT |
| | | TCCGGTGGCTCCGGCGGTTCTGGGCAGGTCCAGCTCCAGGAGAGCGGTGGAGGC |
| | | TCAGTGCAGGCTGGGGGCTCACTGAGACTGTCCTGCCGCGCCTCCCGCTCTCCG |
| | | TATGGCAACTACTGCCTTGGCTGGTTTCGCCAGAGCACTGGTAAGGAACGTGAG |
| | | GGAGTCGCCGTCATCAACTGGGGGGAGGGATGCTGTATTTCGCTGCACAGCGTG |
| | | AAGGGCCGCTTCACTGTGTCCCAGGACCACGCGAAGAACACAGTGACCCTGCAA |
| | | ATGAATAGTCTCAAACCAGAAGACACTGCTATGTATTACTGTGCTGCCGAATCC |
| | | GTCAGCTCTTTCAGCTGCGGTGGATGGGTGACAAGGCCAGATCGGGTGCCTTAC |
| | | TGGGGTCAGGGCACCCAGGTGACCGTTAGCAGC |
| DR596- | 1241 | CAGGTGCAGTTGCAGGAATCCGGGGGGGGATTGGTGCAACCTGGGGGCTCCCTG |
| hIL27 | | CGCTTGTCCTGTACTGCAAGGGGACTGACCTTCGATGACTCTGTTATGGGCTGG |
| Ra_VH | | TTTCGCCAAGCTCCGGGTAAAGGGAGGGAAGCAGTGTCCTGTATCAGCTCCAGC |
| H24 | | GGGGCCAATGCTTTCTACGCGGATAGCGTGAAGGGTCGTTTTACTATTAGCCGC |
| | | GACAATGCAAAGAACACCCTGTACTTGCAGATGAACTCACTCAAGCCCGAAGAT |
| | | ACAGCCACTTACTATTGTAAGCGCGGCCACGCCTGCGCGGGATATTACCCAATC |
| | | CCTTACGATGACTACTGGGGCCAGGGCACACAGGTTACCGTGAGTTCTGGCGGT |
| | | GGCAGTCAGGTGCAGCTCCAGGAGAGCGGGGGTGGCTTGGTGCAGCCGGGAGGG |
| | | TCTCTGAGGGTGTCCTGTGCTGCCTCAGGTTTCACCTTTTCCCACTCCGGCATG |
| | | TCCTGGGTGCGCCAGGCCCCCGGCAAGGGTCTGGAATGGGTGAGTACCATCAAC |
| | | AGTGGGGGCGCAAGTACCTACTATACCGACTCTGTTAAGGGACGCTTCACTATC |
| | | AGCCGCGACAACGCTAAGAACATGCTGTACCTCCAGTTGAACAGCCTTAAAACC |
| | | GAAGACACGGCCATGTATTACTGCGCGAAAGGGGGCTCTGGCTACGGCGACGCG |
| | | AGCAGGATGACCAGTCCCGGTTCCCAGGGCACACAGGTGACTGTTAGCAGC |
| DR596- | 1242 | CAGGTGCAGTTGCAGGAATCTGGGGGTGGCCTGGTGCAACCTGGGGGCAGCCTG |
| hIL27 | | CGCCTGAGCTGCACCGCCAGCGGTCTGACCTTCGACGATTCCGTGATGGGATGG |
| Ra_VH | | TTCCGCCAGGCTCCTGGAAAGGGAAGGGAAGCTGTGAGCTGCATCAGCTCATCC |
| H24 | | GGCGCGAACGCCTTCTACGCCGATTCTGTGAAGGGCCGTTTTACCATCAGCAGA |
| | | GACAACGCCAAGAACACTCTTTATTTGCAGATGAACTCCCTGAAACCAGAGGAT |
| | | ACAGCAACCTATTACTGTAAGAGGGGTCACGCCTGCGCCGGTTATTACCCCATT |
| | | CCGTATGACGATTACTGGGGACAGGGCACCCAGGTAACCGTGTCCTCAGGTGGC |
| | | AGTGGTGGCTCCGGCGGGAGCGGTCAGGTGCAACTCCAGGAGAGGGGGGGGGGC |
| | | CTGGTCCAGCCCGGTGGCTCCCTGCGTCTGTCCTGCGCGGCCTCTGGGTTCACT |
| | | TTCAGCCATTCCGGCATGAGCTGGGTCCGCCAGGCCCCTGGCAAGGGTCTGGAG |
| | | TGGGTTAGCACGATCAACTCAGGGGGGGCCAGCACTTATTACACCGACTCCGTG |
| | | AAGGGACGCTTCACCATCTCTCGTGACAATGCTAAGAACATGCTGTATTTGCAG |
| | | CTGAACTCCCTGAAGACTGAGGACACTGCCATGTACTATTGCGCCAAAGGTGGG |
| | | TCCGGTTACGGTGACGCGAGCAGAATGACCTCACCTGGGAGCCAGGGCACGCAG |
| | | GTGACCGTGTCCTCT |
| hIL27 | 1243 | CAGGTGCAGCTGCAAGAGTCTGGCGGGGGCCTGGTGCAGCCTGGAGGCAGCCTG |
| Ra_VH | | CGCCTGAGCTGTGCGGCATCAGGATTCACGTTTAGCTCCTACCCTATGTCTTGG |
| H3- | | GTCAGACAGGCACCGGGAAAGGGTTTGGAGTGGATCTCCACAATTTCCGGTGGC |
| DR591 | | GGCGACACCACTCTCTACGCTGATTCTGTGAAGGGCCGCTTCACCTCATCTCGC |
| | | GACAACGCTAAGAACACCTTGTACCTGCAACTGAACTCTCTCAAGACCGAGGAC |
| | | GCCGCAATTTACTATTGCGCCAAGCGTATTGATTGCAATTCCGGTTACTGCTAC |
| | | CGTAGGAACTACTGGGGCCAGGGTACTCAGGTCACAGTGTCTAGTGGTGGCGGT |
| | | TCCCAAGTGCAGCTCCAGGAATCTGGAGGTGGGTCTGTGCAGGGTGGAGGCAGC |
| | | CTGAGGCTGAGCTGTACCGCATCCGGGGCAATCGCCTCTGGCTACATCGACTCC |
| | | CGCTGGTGCATGGCCTGGTTTCGCCAGGCTCCCGGCAAAGAGCGCGAGGGCGTG |
| | | GCCGCAATCTGGCCTGGGGGGGGCCTGACCGTTTATGCCGACAGCGTCAAGGGA |
| | | CGCTTCACTATTAGCCGGGACCACGCTAAAAACACACTGTACCTTCAGATGAAT |
| | | AACCTCAAACCGGAGGACACTGCGATGTACTATTGCGCGGCAGGATCACCCCGC |
| | | ATGTGTCCCTCCCTGGAGTTCGGTTTCGATTACTGGGGGCAGGGCACCCAGGTT |
| | | ACCGTAAGTTCC |
| hIL27 | 1244 | CAAGTCCAGCTCCAAGAGAGCGGCGGTGGCCTGGTGCAACCTGGGGGCTCCCTG |
| Ra_VH | | CGCCTGTCTTGCGCTGCATCCGGGTTCACCTTCTCTAGCTACCCGATGTCTTGG |
| H1- | | GTGCGTCAGGCTCCGGGTAAGGGCCTGGAATGGATCAGTACCATCTCAGCCGGA |
| DR591 | | GGCGATACCACTCTGTATGCCGATTCTGTGAAAGGCCGGTTTACCTCCAGCCGT |
| | | GATAACGCAAAGAACACTTTGTACCTCCAGCTGAACTCATTGAAGACTGAGGAT |
| | | GCGGCCATTTATTACTGTGCAAAACGCATTGATTGTAATTCCGGGTACTGCTAC |
| | | CGCCGTAACTATTGGGGTCAAGGTACACAGGTAACCGTGTCCAGTGGTGGCTCT |
| | | GGCGGTAGTGGTGGGTCTGGCCAGGTGCAGCTTCAGGAAAGTGGTGGAGGTAGC |
| | | GTGCAAGCTGGAGGCTCCCTGAGGCTCAGCTGTACCGCGTCTGGAGCAATCGCC |
| | | TCCGGCTACATCGACAGCCGCTGGTGCATGGCTTGGTTCCGCCAGGCTCCAGGG |
| | | AAGGAGAGGGAAGGTGTGGCTGCAATTTGGCCTGGAGGGGGCTTGACGGTGTAT |
| | | GCTGATAGCGTCAAGGGACGCTTCACCATTTCCAGGGATCACGCGAAAAACACC |
| | | CTGTACCTGCAAATGAATAACCTGAAACCCGAGGACACCGCAATGTATTACTGC |
| | | GCTGGGGGTTCCCCGCGCATGTGTCCTTCTCTGGAGTTCGGGTTCGATTACTGG |
| | | GGGCAGGGTACACAAGTGACTGTGTCCTCT |
| hIL27 | 1245 | CAGGTGCAGCTCCAGGAATCAGGCGGTGGACTGGTCCAACCCGGAGGGTCCCTG |
| Ra_VH | | CGGCTTTCCTGCGCCGCAAGGGGATTTACTTTCTCCTCTTACCCAATGAGCTGG |
| H1- | | GTGCGCCAAGCGCCAGGGAAGGGTCTGGAGTGGATCTCTACAATTAGTGCGGGC |
| DR592 | | GGTGACACCACACTCTACGCTGACAGCGTCAAGGGTCGGTTTACCTCCTCTCGC |
| | | GACAACGCCAAGAACACTCTGTATCCTCAGCTGAACTCCCTGAAAACCGAAGAC |
| | | GCGGCAATTTACTATTGCGCTAAGCGGATCGACTGCAACTCCGGCTACTGTTAC |
| | | CGTCGGAACTACTGGGGCCAGGGCACACAAGTTACCGTCAGCTCCGGTGGGGGT |
| | | TCACAAGTACAGCTCCAGGAGTCCGGGGGGGGCAGTGTGCAGGCTGGTGGCTCC |
| | | CTGCGCCTGTCCTGCACGGCTCCCGGCTTCACGTCCAACTCATGCGGTATGGAT |
| | | TGGTATCGGCAGGCTCCTGGAAAGGAACGCGAATTTGTGAGTTCAATTTCTACT |
| | | GACGGCACCACGGGCTACGCTGACAGCGTCAAGGGCAGATTTACTATTTCCAAA |
| | | GATAAGGCCAAGGATACCGTGTATCTCCAAATGAATAGCTTGAAGCCTGAGGAT |
| | | ACGGGGATGTACTCTTGCAAGACCAAGGACGGCACCATCGCAACTATGGAACTC |
| | | TGTGATTTCGGCTATTGGGGGCAAGGGACACAGGTGACCGTCAGCTCT |
| hIL27 | 1246 | CAAGTACAGCTTCAGGAGTCTGGCGGGGGCCTCGTCCAGCCCGGAGGCAGCCTG |
| Ra_VH | | CGGTTGTCCTGTGCCGCATCCGGGTTCACCTTCTCCTCTTACCCAATGTCTTGG |
| H1- | | GTGCGCCAGGCCCCAGGTAAGGGATTGGAGTGGATCTCCACCATGTCTGCGGGC |
| DR592 | | GGAGACACGACTCCTTACGCCGACAGCGTTAAGGGCCGGTTTACCTCTTCCCGC |
| | | GATAACGCCAAAAACACTCTGTATCTTCAGCTGAACAGTCTGAAGACCGAGGAT |
| | | GCCGCTATTTATTACTGTGCTAAGCGCATCGACTGCAACAGCGGATATTGCTAC |
| | | CGGAGGAACTACTGGGGGCAGGGCACCCAGGTTACTGTCAGCTCAGGCGGTTCC |
| | | GGGGGCTCTGGGGGCTCCGGGCAGGTGCAGCTCCAGGAGTCCGGCGGAGGCAGT |
| | | GTCCAAGCAGGAGGTTCACTGCGTCTGTCCTGTACCGCCCCCGGTTTTACATCA |
| | | AACTCATGTGGCATGGACTGGTATCGCCAGGCCCCCGGTAAGGAACGCGAATTT |
| | | GTCTCAAGCATTTCCACTGACGGCACCACTGGTTACGCTGACTCCGTGAAGGGC |
| | | CGTTTCACAATCTCCAAGGACAAGGCCAAGGACACCGTGTACCTTCAGATGAAC |
| | | AGCTTGAAGCCAGAAGACACGGGTATGTACTCTTGCAAGACCAAGGACGGGACC |
| | | ATCGGTACAATGGAGTTGTGTGACTTCGGTTACTGGGGGCAGGGCACGCAAGTC |
| | | ACCGTTTCCTCC |
| hIL27 | 1247 | CAGGTTCAGCTCCAGGAAAGTGGAGGCGGTCTCGTGCAGCCGGGCGGATCTCTT |
| Ra_VH | | AGACTGAGCTGTGCGGCCAGCGGCTTCACTTTCTCTAGCTACCCCATGTCCTGG |
| H1- | | GTGCGGCAGGCTCCGGGTAAGGGCCTGGAGTGGATCTCCACCATTTCGGCAGGA |
| DR593 | | GGCGATACTACCCTGTATGCCGACTCTGTCAAAGGGCGGTTCACCTCATCCCGC |
| | | GACAACGCCAAGAACACTCTGTACCTCCAGCTCAACTCCCTGAAGACCGAAGAC |
| | | GCCGCTATCTACTATTGTGCCAAGCGCATCGACTGTAATTCCGGCTACTGTTAT |
| | | AGACGTAACTACTGGGGTCAGGGAACACAGGTTACTGTGTCCCCTGGCGGAGGT |
| | | TCTCAGGTTCAGCTTCAGGAGTCAGGTGGAGGTTCTGTGCAGGCAGGGGGGAGG |
| | | TTGAGACTCTCCTGTGCGGCCAGCGGATACCCTTACTCAAATGGCTATATGGGA |
| | | TGGTTCAGGCAGGCTCCCGGAAAGGAGAGGGAGGGCGTGGCGACTATCTACACA |
| | | GGGGACGGTAGGACTTATTACGCAGACAGCGTCAAAGGGAGGTTTACTATCTCA |
| | | CGTGACAATGCGAAGAACACAGTGGACGTCCAAATGTCCAGCCTGAAGCCAGAG |
| | | GATACTGCCATGTATTACTGTGCAGCTAGGGGGGCACCCCTCTACTCTAGTGGC |
| | | TCTCCCTTGACACGGGCGAGGTACAATGTTTGGGGTCAGGGGACCCAGGTGACT |
| | | GTGTCCTCA |
| hIL27 | 1248 | CAGGTCCAGTTGCAGGAAAGTGGAGGCGGACTGGTGCAACCCGGAGGCAGCCTC |
| Ra_VH | | CGTTTGTCCTGTGCGGCTTCCGGCTTCACGTTCAGCTCCTACCCAATGTCTTGG |
| H1- | | GTGCGGCAAGCGCCCGGCAAGGGGCTTGAGTGGATCTCTACAATCAGCGCAGGA |
| DR593 | | GGTGATACTACCCTGTATGCCGACTCTGTCAAGGGCAGATTCACCTCCAGCCGC |
| | | GACAACGCCAAGAACACCCTTTATCTCCAACTGAACAGCCTCAAGACCGAAGAT |
| | | GCCGCGATTTACTATTGTGCTAAGCGCATTGACTGCAACTCCGGCTATTGCTAC |
| | | AGGCGCAACTACTGGGGCCAGGGCACTCAGGTGACTGTGTCCAGCGGCGGTTCT |
| | | GGAGGCTCCGGGGGCAGCGGCCAGGTGCAGCTCCAGGAGTCTGGTGGAGGCTCC |
| | | GTTCAGGCCGGAGGTTCCCTTCGCCTGTCTTGTGCTGCAAGCGGCTACCCATAC |
| | | TCTAACGGCTACATGGGCTGGTTCCGGCAAGCGCCGGGTAAAGAACGCGAAGGG |
| | | GTGGCCACGATCTACACCGGGGATGGCAGAACCTATTACGCCGATAGCGTGAAG |
| | | GGTCGTTTCACTATTTCCCGTGACAATGCGAAGAACACCGTGGACCTCCAGATG |
| | | AGTTCCTTGAAGCCTGAGGATACAGCTATGTACTATTGCGCCGCTCGGGCAGCC |
| | | CCACTCTATTCCAGCGGTAGTCCCCTGACCCGTGCTCGCTACAACGTATGGGGA |
| | | CAGGGTACACAGGTGACTGTGTCCTCC |
| hIL27 | 1249 | CAGGTCCAGCTCCAGGAATCCGGTGGGGGCCTCGTGCAGCCCGGAGGTTCTCTG |
| Ra_VH | | CGCCTGTCTTGTGCCGCGAGCGGATTTACTTTCAGCTCCTACCCCATGTCCTGG |
| H1- | | GTGCGTCAAGCCCCAGGTAAGGGGCTGGAGTGGATTTCTACTATTAGCGCTGGA |
| DR594 | | GGCGACACAACCCTGTATGCCGACAGCGTGAAGGGTCGCTTTACTTCTAGCCGC |
| | | GACAATGCAAAGAACACACTGTACCTTCAGCTCAACTCCCTGAAAACTGAAGAC |
| | | GCGGCCATCTATTACTGCGCCAAGCGGATTGATTGTAACAGCGGATATTGCTAC |
| | | AGACGCAACTACTGGGGCCAGGGGACCCAGGTGACAGTGTCAAGCGGGCGGGGG |
| | | TCTCAGGTGCAGCTTCAGGAGAGCGGGGGAGGCAGCGTCCAGGCTGGTGGCTCC |
| | | CTGAGGCTTTCTTGTGTGGCCTCCGCCAGTACCTACTGCACCTATGATATGCAC |
| | | TGGTATCGGCAAGCGCCGGGCAAAGGGAGAGAGTTTGTGTCCGCTATCGACTCA |
| | | GATGGCACAACCAGATACGCCGATAGCGTTAAGGGCAGGTTCACTATCAGTCAG |
| | | GGGACCGCTAAGAACACCGTGTATCTCCAGATGAATAGCCTCCAGCCAGAGGAT |
| | | ACCGCCATGTACTATTGTAAGACAGTGTGTGTTGTCGGATCACGCTGGAGCGAC |
| | | TACTGGGGACAGGGCACCCAGGTGACCGTGTCCTCT |
| hIL27 | 1250 | CAGGTCCAGTTGCAAGAATCCGGCGGTGGCCTCGTGCAGCCTGGGGGGTCACTG |
| Ra_VH | | CGTTTGTCTTGTGCCGCTAGTGGCTTCACATTTAGCTCCTACCCCATGAGCTGG |
| H1- | | GTCCGGCAGGCCCCTGGCAAAGGCCTGGAGTGGATCAGTACCATCTCCGCAGGG |
| DR594 | | GGCGACACAACCCTGTATGCGGACTCTGTGAAGGGTAGATTCACATCATCCAGG |
| | | GACAACGCCAAGAACACACTTTATCCCCAGCTTAACTCTCTGAAGACAGAGGAT |
| | | GCAGCCATCTACTATTGCGCTAAGCGTATCGACTGCAACTCCGGTTATTGTTAC |
| | | CGTCGCAATTACTGGGGACAGGGAACACAGGTGACTGTTTCCTCTGGAGGCTCC |
| | | GGTGGCTCCGGGGGCAGCGGCCAGGTGCAGCTCCAGGAGTCTGGAGGCGGAAGC |
| | | GTGCAGGCTGGCGGTTCTCTGCGCCTGAGCTGTGTCGCAAGTGCCTCTACCTAC |
| | | TGCACTTACGACATGCACTGGTATCGGCAAGCGCCCGGAAAAGGCAGGGAGTTT |
| | | GTTAGCGCCATTGATTCCGACGGTACTACCCGCTATGCCGATAGCGTGAAAGGC |
| | | AGGTTTACTATCAGCCAGGGCACTGCGAAGAACACCGTGTACCTCCAGATGAAC |
| | | TCCCTCCAGCCCGAGGACACCGCCATGTATTACTGCAAAACCGTCTGCGTCGTG |
| | | GGATCACGTTGGTCTGACTATTGGGGGCAGGGGACTCAGGTCACTGTGTCCAGC |
| hIL27 | 1251 | CAGGTGCAGCTTCAGGAGAGCGGGGGGGGTCTCGTGCAGCCTGGAGGCCCTCTG |
| Ra_VH | | CGCCTGTCCTGTGCCGCGAGCGGATTCACTTTCTCTTCATACCCGATGTCCTGG |
| H1- | | GTGCGCCAGGCCCCCGGCAAGGGACTGGAGTGGATCAGCACGATCTCCGCCGGA |
| DR595 | | GGCGACACAACCCTGTATGCCGACTCTGTTAAGGGGAGGTTCACCAGTAGCCGT |
| | | GACAATGCCAAGAACACTCTGTATCTGCAACTGAACTCTCTCAAGACTGAGGAT |
| | | GCCGCTATCTATTACTGCGCGAAGCGCATTGACTGCAACAGTGGCTACTGCTAC |
| | | CGCAGGAACTATTGGGGCCAGGGCACCCAGGTTACCGTTAGTTCTGGAGGCGGA |
| | | TCACAAGTGCAGCTGCAAGAGTCTGGAGGTGGCAGTGTGCAAGCAGGTGGCTCT |
| | | CTGACTCTGTCCTGCGCTGCCAGCGAGTATGCTTACTCTACGTGCAACATGGGC |
| | | TGGTATCGCCAGGCCCCTGGGAAAGAGCGGGAACTGGTTTCCGCGTTCATCTCC |
| | | GACGGCTCCACTTATTACGCCGACTCCGTGAAGGGGAGATTCACCATCACTCGG |
| | | GACAACGCAAAAAATACTGTCTATCCCCAGATGAAGCCCCTCAAGCCTGAGGAT |
| | | ACAGCTATCTACTATTGCTCTGCTAACTGCTATCGTCGCCTGCGCAACTATTGG |
| | | GGACAGGGCACACAGGTGACAGTTAGCAGC |
| hIL27 | 1252 | CAGGTGCAGTTGCAGGAATCTGGGGGGGGTCTTGTCCAGCCCGGTGGCTCACTC |
| Ra_VH | | CGCCTGAGCTGCGCTGCCAGCGGTTTCACTTTCAGTTCCTATCCTATGAGTTGG |
| H1- | | GTGCGGCAGGCCCCAGGCAAAGGACTGGAGTGGATCTCTACAATCTCAGCTGGT |
| DR595 | | GGCGATACCACTCTTTATGCCGATTCTGTGAAGGGTCGTTTCACTTCTTCCAGA |
| | | GACAACGCGAAGAACACGTTGTATTTGCAGCTGAACAGTCTGAAAACCGAAGAC |
| | | GCGGCCATCTATTACTGTGCCAAGAGGATTGATTGTAACAGCGGGTATTGTTAT |
| | | CGCCGTAACTATTGGGGGCAGGGCACTCAAGTTACCGTTAGCTCCGGTGGCTCC |
| | | GGGGGTAGCGGGGGAAGCGGCCAGGTCCAGCTGCAAGAGTCTGGAGGGGGTTCC |
| | | GTTCAGGGGGGAGGGTCTCTGACCCTGTCTTGCGCGGCCTCAGAATACGCCTAC |
| | | TCCACCTGTAATATGGGTTGGTACAGACAGGCCCCTGGCAAAGAGAGGGAACTC |
| | | GTTAGCGCTTTCATCTCCGATGGGTCCACTTATTACGCGGACAGCGTCAAGGGC |
| | | CGCTTCACCATCACACGCGATAACGCCAAGAATACGGTTTATCTCCAGATGAAC |
| | | TCCCTCAAGCCCGAGGATACTGCGATCTATTACTGTTCCGCGAACTGCTACAGA |
| | | CGGCTGCGTAACTATTGGGGCCAGGGGACTCAGGTTACCGTGTCCAGC |
| hIL27 | 1253 | CAGGTTCAGCTCCAGGAGAGCGGTGGAGGTTTGGTCCAACCGGGAGGCAGCCTG |
| Ra_VH | | AGACTCAGTTGTGCCGCTTCTGGCTTCACTTTCTCCTCTTACCCTATGTCCTGG |
| H1- | | GTCAGACAAGCACCGGGGAAGGGTCTGGAATGGATCTCCACCATTTCAGCTGGA |
| DR596 | | GGCGATACCACATTGTACGCCGACTCCGTGAAGGGACGCTTCACCTCTTCCCGC |
| | | GATAATGCCAAGAACACCCTGTATTTGCAGTTGAATAGCCTGAAGACCGAGGAT |
| | | GCCGCTATTTACTATTGTGCTAAGCGGATCGACTGTAACAGCGGTTACTGTTAC |
| | | AGGCGCAACTACTGGGGTCAAGGCACCCAGGTAACCGTCAGCTCCGGCGGTGGG |
| | | TCCCAGGTGCAGCTCCAGGAGTCAGGAGGCGGACTCGTGCAACCCGGTGGCTCC |
| | | CTCCGCCTCAGCTGTACCGCTTCCGGTCTCACCTTTGATGACTCAGTTATGGGC |
| | | TGGTTTAGGCAAGCGCCGGGCAAGGGCAGAGAAGCTGTGTCTTGTATCTCCTCA |
| | | TCCGGTGCTAACGCCTTCTACGCTGACAGCGTGAAGGGTCGCTTTACAATCAGT |
| | | AGAGATAACGCCAAAAACACTCTTTACTTGCAGATGAACTCTCTCAAGCCTGAG |
| | | GATACCGCCACTTATTACTGTAAGAGGGGTCATGCTTGCGCTGGCTATTACCCC |
| | | ATCCCCTATGATGACTATTGGGGCCAGGGCACCCAGGTGACTGTGTCTAGT |
| hIL27 | 1254 | CAGGTGCAGCTTCAGGAGAGGGGCGGAGGCCTCGTGCAGCCAGGAGGCTCCCTG |
| Ra_VH | | AGACTCTCCTGCGCCGCAAGCGGATTTACATTTTCTAGCTACCCGATGTCCTGG |
| H1- | | GTGAGACAAGCGCCCGGCAAAGGCCTTGAATGGATCTCTACTACTTCTGCTGGA |
| DR596 | | GGCGACACAACCCTGTATGCCGATAGGGTTAAGGGACGTTTCACCTCTTCCAGA |
| | | GACAATGCTAAGAACACCCTGTACTTGCAGCTGAACTCCCTCAAGACGGAGGAT |
| | | GCAGCGATCTACTATTGCGCGAAGAGGATTGACTGTAACAGTGGCTATTGTTAG |
| | | AGGCGCAATTACTGGGGCCAGGGAACTCAGGTGACCGTGTCCTCTGGGGGTTCC |
| | | GGGGGATCAGGAGGCAGTGGCCAGGTGCAGTTGCAGGAAAGTGGCGGAGGGCTG |
| | | GTGCAACCAGGCGGATCTTTGAGATTGTCCTGCACCGCCTCCGGCCTGACATTT |
| | | GACGATTCAGTGATGGGCTGGTTCCGTCAGGCACCGGGCAAGGGCCGGGAGGGC |
| | | GTGAGTTGTATCAGCTCCTCAGGCGCAAACGCCTTCTACGCCGACAGTGTTAAG |
| | | GGCAGGTTCACTATCTCCAGAGACAATGCAAAGAACACTCTGTACCTTCAGATG |
| | | AACAGCCTGAAGCCAGAGGACACAGCCACATACTATTGCAAGCGCGGCCACGCT |
| | | TGCGCCGGGTACTATCCCATCCCCTACGACGATTACTGGGGACAGGGCACTCAG |
| | | GTCACAGTGAGTAGC |
| hIL27 | 1255 | CAAGTACAACTTCAGGAATCTGGGGGCGGTCTCGTTCAGCCTGGGGGCTCTCTG |
| Ra_VH | | AGGCTGTCTTGCGCCGCGAGCGGATTTACTTTTTCCCTCAGCGGCATGAGCTGG |
| H2- | | GTCAGACAGGCCCCTGGCAAAGGGCTGGAATGGGTGTCCGCCATCTCCAGCGGG |
| DR591 | | GGCGCTTCTACATACTATACTGACAGCGTGAAGGGCAGGTTCACTATTTCCCGC |
| | | GACAATGCCAAGAACATTCTTTACCTTCAGCTGAACTCCCTCAAAACAGAGGAC |
| | | ACCGCGATGTATTACTGTGCTAAGGGCGGGAGCGGTTATGGCGATGCGAGTCGG |
| | | ATGACCTCCCCCGGCAGTCAGGGAACTCAGGTCACTGTCAGCTCTGGGGGGGGG |
| | | TCCCAGGTTCAACTCCAGGAGTCAGGAGGGGGGTCTGTTCAGGCTGGGGGTAGC |
| | | CTGAGGCTGTCCTGCACCGCCTCTGGAGCCATCGCCAGCGGCTATATTGATTCA |
| | | CGCTGGTGTATGGCGTGGTTCCGCCAGGCTCCTGGCAAAGAAAGGGAAGGTGTA |
| | | GCCGCTATTTGGCCTGGAGGCGGTCTGACCGTCTATGCCGATAGCGTGAAGGGC |
| | | AGGTTTACTATCTCTCGGGATCACGCGAAGAACACCCTGTATCTTCAGATGAAC |
| | | AATCTCAAGCCTGAGGACACAGCTATGTATTACTGCGCAGCGGGTTCCCCGCGG |
| | | ATGTGCCCCAGCCTGGAGTTCGGATTCGATTACTGGGGGCAAGGCACCCAAGTC |
| | | ACTGTTTCTAGC |
| hIL27 | 1256 | CAAGTTCAGCTCCAGGAGTCCGGTGGGGGACTCGTTCAGCCTGGAGGCAGTCTG |
| Ra_VH | | AGGCTGTCTTGTGCCGCGTCAGGGTTCACCTTCAGCCTGTCTGGGATGAGCTGG |
| H2- | | GTCCGCCAGGCCCCAGGCAAGGGCCTGGAATGGGTGTCAGCCATCAGCTCCGGC |
| DR591 | | GGTGCCTTCTACTTATACACTGACTCAGTGAAGGGTCGTTTTACCATCTCCCGC |
| | | GACAACGCCAAGAACATTCTGTATCTCCAGCTCAACAGCCTGAAAACGGAGGAC |
| | | ACCGCTATGTATTACTGTGCTAAGGGTGGCTCAGGGTACGGGGACGCTTCTCGC |
| | | ATGACATCTCCTGGCTCCCAGGGCACCCAGGTGACTGTCAGCTCAGGAGGCAGC |
| | | GGAGGCAGTGGCGGATCTGGGCAGGTCCAGCTCCAAGAGTCTGGAGGGGGTTCA |
| | | GTGCAGGCTGGAGGTTCTCTTAGGCTGAGCTGTACCGCCAGCGGAGCCATCGCT |
| | | TCTGGGTATATTGACAGCCGCTGGTGCATGGCTTGGTTCCGGCAAGCTCCAGGT |
| | | AAGGAGAGGGAGGGAGTGGCAGCCATTTGGCCCGGAGGCGGACTCACAGTGTAT |
| | | GCCGATAGCGTTAAGGGCAGATTTACCATCAGCCGCGATCACGCAAAAAACACA |
| | | CTCTACCTTCAGATGAATAACCTGAAGCCGGAGGACACCGCCATGTATTACTGT |
| | | GCCGCTGGCTCTCCACGCATGTGCCCTAGCCTGGAGTTCGGTTTTGACTACTGG |
| | | GGTCAAGGCACACAGGTGACCGTGTCTAGT |
| hIL27 | 1257 | CAGGTCCAGTTGCAGGAATCTGGAGGCGGACTGGTGCAACCTGGCGGTTCCCTG |
| Ra_VH | | CGGCTGTCCTGTGCAGCCAGTGGCTTTACCTTCAGCCTGAGCGGCATGTCCTGG |
| H2- | | GTGAGACAGGCCCCCGGAAAGGGTCTGGAATGGGTGAGTGCCATCAGCTCCGGC |
| DR592 | | GGAGCCAGTACCTATTACACCGATAGTGTTAAGGGGCGCCTTTACCATCCCCGT |
| | | GACAACGCTAAGAATATCCTTTATCTCCAACTGAACAGCCTGAAGACCGAAGAC |
| | | ACCGCCATGTATTACTGTGCCAAGGGGGCTCTGGATATGGCCGATGCCAGCCGC |
| | | ATGACCAGTCCTGGGTCCCAAGGCACACAGGTAACAGTGTCTAGCGGGGGAGGC |
| | | AGCCAGGTGCAGCTGCAAGAAAGCGGCGGTGGCAGTGTCCAAGCTGGGGGCAGC |
| | | TTGCGCCTCTCTTGCACTGCGCCCGGTTTCACCTCTAATAGCCGTGGCATGGAT |
| | | TGGTATCGCCAGGCACCGGGCAAGGAGAGGGAGTTCGTGAGTTCCATCAGCACC |
| | | GACGGCACTACGGGATATGCCGACAGCGTTAAGGGCCGCTTCACCATCTCAAAG |
| | | GACAAGGCCAAGGATACTGTGTACCCGCAAATGAACAGTCTGAAGCCGGAGGAT |
| | | ACCGGGATGTACTCTTGCAAGACCAAGGACGGAACCATCGCAACTATGGAGCTG |
| | | TGTGACTTCGGCTACTGGGGCCAGGGAACCCAGGTGACAGTCTCTTCC |
| hIL27 | 1258 | CAGGTGCAGCTTCAGGAGTCCGGCGGTGGCCTGGTCCAGCCGGGCGGAAGCCTG |
| Ra_VH | | CGCCTTTCCTGCGCCGCGTCTGGCTTCACCTTCTCTCTCAGGGGTATGAGTTGG |
| H2- | | GTCCGCCAGGCCCCAGGGAAGGGCCTGGAGTGGGTGTCTGCTATCTCTAGCGGA |
| DR592- | | GGCGCTAGTACCTATTACACAGATTCCGTCAAGGGGCGCTTTACAATCTCACGC |
| | | GATAACGCCAAGAACATCCTGTACCTTCAGCTGAACAGCCTGAAAACCGAGGAT |
| | | ACCGCTATGTACTATTGCGCTAAGGGAGGCTCTGGCTACGGAGACGCTTCACGT |
| | | ATGACCAGTCCTGGCAGCCAGGGAACACAGGTCACAGTCAGCTCCGGGGGTAGC |
| | | GGTGGCTCCGGCGGGAGCGGCCAGGTTCAACTCCAGGAATCTGGGGGGGGTTCC |
| | | GTCCAGGCTGGGGGTTCTCTGAGACTGAGCTGCACAGCCCCTGGGTTCACCTCC |
| | | AACAGTTGCGGAATGGATTGGTATCGTCAGGCCCCCGGCAAGGAGAGAGAGTTT |
| | | GTTAGCTCTATCTCCACGGACGGGACCACTGGTTATGCTGACAGCGTTAAGGGC |
| | | CGTTTTACTATCTCCAAGGACAAGGCCAAAGACACCGTGTACTTGCAGATGAAC |
| | | TCTCTGAAGCCCGAGGATACCGGAATGTACTCATGCAAAACCAAAGATGGTACT |
| | | ATTGCTACTATGGAGCTGTGTGACTTCGGCTATTGGGGCCAGGGGACCCAGGTG |
| | | ACTGTGTCCTCT |
| hIL27 | 1259 | CAGGTTCAACTGCAAGAGTCTGGAGGCGGTCTCGTACAGCCAGGTGGGAGCCTC |
| Ra_VH | | CGCCTGTCCTGTGCAGCCAGCGGTTTCACCTTGTCCCTCTCCGGTATGTCCTGG |
| H2- | | GTGCGCCAGGCCCCTGGAAAAGGCTTGGAGTGGGTGTCTGCCATCAGCTCAGGA |
| DR593 | | GGCGCGTCCACATATTACACCGACAGTGTCAAGGGCCGGTTCACCATCTCCAGG |
| | | GACAATGCGAAGAATATCCTCTACTTGCAGCTGAACTCCCTCAAAACTGAGGAC |
| | | ACTGCAATGTATTACTGCGCTAAAGGGGGCAGCGGATATGGGGATGCTTCCCGG |
| | | ATGACATCCCCAGGCAGCCAGGGCACCCAAGTGACCGTGTCTTCCGGTGGGGGG |
| | | TCCCAGGTGCAGCTCCAGGAAAGCGGAGGGGGAAGCGTGCAGGCAGGGGGTAGC |
| | | CTTCGTCTGTCTTGTGCAGCCAGCGGCTACCCGTATTCTAATGGATACATGGGC |
| | | TGGTTCAGGCAAGCCCCTGGCAAGGAACGCGAGGGCGTGGCCACGATCTATACC |
| | | GGAGACGGCAGGACTTACTATGCCGATAGCGTGAAAGGGCGCTTCACCATCTCA |
| | | CGTGACAACGCGAAAAATACCGTGGATGTTCAGATGTCCTCTCTCAAACCCGAG |
| | | GACACCGCGATGTACTATTGCGCCGCACGGGCTGCCCCTCTGTACTCATCTGGC |
| | | TCACCGTTGACTCGGGCAAGATATAACGTGTGGGGTCAGGGCACACAGGTCACC |
| | | GTGTCTTCC |
| hIL27 | 1260 | CAGGTCCAACTCCAGGAAAGCGGGGCGGCCCTGGTGCAGCCTGGTGGGTCCCTC |
| Ra_VH | | AGACTGTCTTGCGCCGCTTCTGGCTTCACCTTTTCTTTGTCCGGGATGTCTTGG |
| H2- | | GTCCGCCAAGCGCCGGGCAAGGGCCTGGAGTGGGTGTCCGCCATTTCATCTGGT |
| DR593 | | GGAGCTTCAACTTATTACACCGACTCTGTGAAGGGTCGTTTTACCATCTCCCGT |
| | | GACAACGCGAAGAACATCCTGTATTTGCAGCTGAACTCCTTGAAAACCGAGGAT |
| | | ACGGCCATGTACTATTGTGCAAAGGGCGGTTCCGGTTACGGCGACGCTTCACGC |
| | | ATGACCTCCCCCGGTTCCCAGGGCACTCAGGTGACCGTCTCAAGTGGGGGTAGC |
| | | GGTGGCTCTGGTGGCAGCGGACAGGTCCAGCTTCAGGAATCAGGCGGAGGGAGT |
| | | GTGCAAGCAGGCGGATCTCTGAGACTGAGCTGTGCTGCCAGCGGTTACCCCTAC |
| | | TCTAACGGCTATATGGGTTGGTTCAGGCAAGCGCCTGGTAAGGAGCGCGAGGGT |
| | | GTGGCCACGATCTATACCGGGGATGGCAGGACCTACTATGCCGACTCTGTGAAG |
| | | GGTCGGTTCACCATCAGCCGGGACAACGCGAAGAACACTGTGGATTTGCAGATG |
| | | TCTTCCCTGAAACCCGAGGACACCGCCATGTATTACCGCGCTGCAAGAGCCGCT |
| | | CCCCTGTATAGCTCTGGATCTCCTCTGACCCGTGCAAGATACAATGTATGGGGT |
| | | CAGGGAACCCAGGTCACCGTCAGCAGT |
| hIL27 | 1261 | CAGGTGCAGCTTCAGGAGTCAGGTGGGGGCCTGGTCCAGCCAGGCGGTTCCTTG |
| Ra_VH | | CGCCTGTCATGCGCCGCTAGTGGGTTCACTTTTAGCCTGTCCGGCATGTCCTGG |
| H2- | | GTTCGGCAAGCACCCGGCAAGGGTTTGGAGTGGGTGTCCGCCATTTCTTCCGGG |
| DR594 | | GGCGCATCTACCTATTACACTGATTCTGTCAAGGGGCGCTTTACCATTTCTCGT |
| | | GACAACGCTAAAAACATTCTGTATCTGCAACTTAACTCACTGAAGACCGAGGAT |
| | | ACAGCAATGTACTATTGCGCCAAGGGTGGCTCCGGGTACGGCGATGCGTCACGG |
| | | ATGACATCTCCTGGGTCCCAGGGAACTCAGGTGACCGTGTCCTCAGGGGGGGGC |
| | | AGTCAAGTGCAGCTCCAGGAGAGCGGAGGTGGCTCTGTGCAAGCTGGGGGAAGT |
| | | CTCCGCTTGTCTTGTGTGGCCTCCGCAAGCACATATTGCACCTACGACATGCAC |
| | | TGGTATAGGCAGGCTCCCGGCAAGGGGGGGGAGTTTGTTTCCGCAATCGACAGC |
| | | GACGGCACCACTCGCTATGCCGATAGCGTGAAAGGTCGTTTTACCATCTCCCAG |
| | | GGGACCGCAAAGAACACAGTCTACTTGCAGATGAACTCCCTTCAGCCTGAGGAC |
| | | ACCGCTATGTATTACTGTAAGACCGTGTGCGTCGTGGGCAGTCGTTGGTCCGAT |
| | | TACTGGGGCCAGGGAACACAGGTGACAGTCAGTTCT |
| hIL27 | 1262 | CAGGTGCAGGTCCAGGAGTCTGGGGGGGCGCCTGGTGCAACCGGGGGCTCACTG |
| Ra_VH | | CGCCTGTCTTGCGCCGCGTCCGGCTTCACATTCAGTCTCTCCGGGATGTCCTGG |
| H2- | | GTGCGCCAAGCTCCGGGCAAAGGCTTGGAGTGGGTATCTGCTATCTCTAGCGGT |
| DR594 | | GGAGCTTCCACATACTATACCGACTCCGTGAAGGGCCGCTTCACCATTTCACGT |
| | | GATAACGCGAAGAACATTCTGTATCTTCAGCTGAACAGCCTGAAAACCGAGGAC |
| | | ACCGCTATGTACTATTGTGCCAAGGGAGGCAGGGGGTATGGGGACGCTTCCCGG |
| | | ATGACCTCCCCCGGTTCTCAGGGTACTCAGGTGACCGTCTCTAGCGGTGGCTCT |
| | | GGTGGCTCCGGCGGATCTGGACAGGTACAGCTCCAGGAGTCAGGTGGAGGGAGT |
| | | GTGCAGGCAGGTGGCTCCCTGCGGTTGTCCTGTGTGGCCAGCGCCTCTACATAT |
| | | TGCACCTACGACATGCACTGGTACAGGCAGGCCCCAGGTAAAGGGCGTGAATTT |
| | | GTGAGTGCCATTGACTCTGATGGAACGACTAGATACGCGGATTCCGTCAAGGGG |
| | | CGGTTTACAATCTCTCAGGGCACCGCCAAGAACACCGTGTATCTCCAAATGAAC |
| | | TCATTGCAGCCTGAGGACACTGCCATGTATTACTGCAAGACCGTGTGCGTCGTG |
| | | GGCTCCCGTTGGTCCGATTATTGGGGCCAGGGCACACAGGTAACCGTATCTTCC |
| hIL27 | 1263 | CAGGTACAGCTCCAGGAATCCGGTGGGGGCTTGGTCCAGCCAGGGGGTAGTCTG |
| Ra_VH | | CGCCTTTCCTGCGCTGCCTCCGGCTTCACCTTCAGCCTGTCAGGCATGTCCTGG |
| H2- | | GTCCGGCAAGCGCCTGGCAAGGGGCTGGAGTGGGTCTCCGCCATTAGCAGTGGT |
| DR595 | | GGAGCGAGCACTTATTACACTGACAGCGTGAAGGGACGCTTTACAATTTCACGT |
| | | GACAACGCCAAGAACATCCTCTACTTGCAGCTCAATTCTCTGAAGACCGAGGAT |
| | | ACCGCAATGTACTATTGCGCCAAGGGGGCTCCGGTTACGGCGGACGCCAGCCGC |
| | | ATGACATCTCCGGGTTCTCAGGGTACACAGGTGACTGTCTCATCCGGGGGGGGG |
| | | AGCCAGGTCCAGCTCCAGGAGTCTGGTGGGGGCTCCGTGCAAGCTGGCGGTTCA |
| | | CTGACACTGAGCTGTGCAGCGTCTGAGTATGCGTACTCTACGTGTAACATGGGA |
| | | TGGTATAGGCAGGCTCCGGGGAAGGAGCGCGAACTCGTGTCAGCTTTTATTAGC |
| | | GACGGCTCCACGTATTACGCAGATAGCGTCAAGGGACGGTTCACCATCACACGG |
| | | GACAATGCGAAGAATACCGTGTATCCCCAGATGAACTCCCTCAAGCCCGAGGAC |
| | | ACCGCAATCTATTACTGTAGCGCAAATTGCTACCGGAGACTGCGGAACTACTGG |
| | | GGCCAGGGCACCCAGGTAACCGTGTCTTCC |
| hIL27 | 1264 | CAGGTGCAGTTGCAGGAATCAGGGGGGGGTCTCGTGCAGCCCGGTGGATCACTG |
| Ra_VH | | AGACTGTCATGTGCTGCCTCCGGCTTCACATTCAGCCTGTCTGGTATGAGCTGG |
| H2- | | GTCCGCCAGGCACCAGGCAAGGGACTGGAGTGGGTGAGTGCTATCTCATCCGGG |
| DR595 | | GGTGCCAGCACCTATTACACCGACAGCGTAAAAGGACGTTTCACTATCTCCCGC |
| | | GATAACGCTAAGAACATCCTGTACCCCCAGCTCAATTCTCTGAAAACAGAAGAT |
| | | ACCGCCATGTATTACTGCGCTAAGGGAGGCAGCGGGTACGGCGATGCGTCCCGC |
| | | ATGACATCCCCCGGAAGTCAGGGAACCCAAGTGACAGTGTCTTCCGGGGGGTCT |
| | | GGCGGTTCCGGGGGTTCTGGCCAAGTGCAGCTCCAGGAATCCGGGGGGGGATCT |
| | | GTGCAGGGGGGGGGTCTCTGACATTGAGCTGCGCGCGCCTCTGAGTATGGTTAC |
| | | TCTACCTGCAACATGGGCTGGTATAGGCAGGCCCCCGGCAAGGAACGCGAACTG |
| | | GTGTCCGCCTTCATTTCCGACGGGTCCACCTATTACGCCGACTCCGTGAAAGGC |
| | | AGGTTCACTATCACCCGCGACAACGCCAAGAATACTGTGTACCTCCAGATGAAT |
| | | AGCTTGAAGCCCGAGGATACCGCGATTTATTACTGCTCCGCAAATTGCTATCGT |
| | | CGGCTCAGAAATTATTGGGGCCAGGGCACACAGGTTACCGTTAGCTCA |
| hIL27 | 1265 | CAAGTCCAACTCCAGGAGTCCGGCGGTGGCCTGGTGCAGCCCGGTGGGTCCCTG |
| Ra_VH | | CGCCTGTCATGTGCTGCCAGTGGTTTCACATTCAGCCTCTCAGGCATGTCTTGG |
| H2- | | GTACGCCAGGCCCCTGGGAAGGGCCTGGAGTGGGTCTCCGCCATGTCCAGCGGT |
| DR596 | | GGCGCAAGTACATATTACACTGATTCCGTGAAGGGTCGTTTCACAATCTCTAGG |
| | | GATAACGCTAAGAATATCCTGTACCTCCAGCTTAACTCCCTGAAGACCGAAGAT |
| | | ACCGCTATGTATTACTGTGCGAAGGGCGGTTCTGGCTATGGGGATGCCTCACGC |
| | | ATGACCTCTCCTGGGTCCCAGGGCACCCAGGTGACTGTCAGCTCAGGCGGGGGC |
| | | AGCCAGGTGCAGCTCCAGGAGTCCGGTGGCGGGCTGGTGCAGCCTGGCGGAAGC |
| | | CTCAGACTGTCATGTACCGCGAGTGGACTGACATTTGATGACAGCGTCATGGGC |
| | | TGGTTCCGGCAGGCCCCAGGAAAGGGGAGGGAAGCTGTGAGCTGTATTTCTTCC |
| | | TCTGGCGCTAACGCCTTCTACGCTGACAGTGTGAAGGGCCGTTTCACCATTTCC |
| | | AGAGACAACGCAAAGAACACTCTGTACCTCCAGATGAACTCACTGAAGCGTGAG |
| | | GACACCGCGACATACTATTGTAAGCGTGGTCACGCTTGTGCTGGCTACTATCCT |
| | | ATCCCATACGATGACTATTGGGGTCAGGGCACACAGGTGACCGTGAGTAGC |
| hIL27 | 1266 | CAGGTCCAGTTGCAGGAATCTGGAGGTGGCCTGGTTCAGCCCGGAGGCAGCCTG |
| Ra_VH | | AGACTGAGCTGCGCTGCGAGCGGATTCACCTTTAGCCTGTCTGGCATGAGCTGG |
| H2- | | GTAAGGCAGGCTCCAGGCAAGGGACTGGAGTGGGTGTCTGCTATCTCCAGCGGA |
| DR596 | | GGCGCTTCCACCTATTACACAGACTCTGTTAAAGGCCGTTTCACTATCAGTAGA |
| | | GACAACGCGAAGAACATCCTCTATCTTCAGCTGAATAGCCTGAAGACAGAGGAT |
| | | ACCGCCATGTATTACTGTGCCAAGGGAGGTAGGGGTTACGGCGACGCCAGCCGC |
| | | ATGACAAGTCCGGGCTCCCAGGGCACACAGGTGACCGTATCCAGCGGTGGCTCC |
| | | GGTGGGAGGGGCGGTTCCGGCCAGGTGCAGCTGCAAGAAAGCGGCGGTGGCCTG |
| | | GTGCAGCCAGGGGGTTCTCTGCGTCTGAGCTGCACCGCGAGTGGGCTGACCTTC |
| | | GACGATTCCGTTATGGGCTGGTTCCGCCAAGCTCCGGGAAAGGGCAGAGAGGCC |
| | | GTATCCTGCATCAGCTCCAGCGGGGCCAACGCCTTCTATGCCGATAGTGTGAAG |
| | | GGACGCTTTACCATTTCTCGCGATAACGCGAAAAACACACTGTATCTTCAGATG |
| | | AACTCACTGAAGCCTGAGGACACCGCCACGTACTATTGTAAACGCGGCCACGCC |
| | | TGCGCTGGTTATTACCCTATTCCATACGACGATTACTGGGGCCAGGGCACCCAG |
| | | GTCACTGTGTCCTCA |
| hIL27 | 1267 | CAAGTGCAGCTCCAGGAGAGCGGAGGTGGCTCAGTGCAGGCAGGCGGTTCACTG |
| Ra_VH | | CGTCTGTCCTGCGTCGCCTCTGGCTACGTTAGGTGTGATTATTTTCTCCCCTCC |
| H3- | | TGGTATCGGCAGGCTCCTGGGAAAGAGCGCGAATTTGTAAGCATCATTGACGGC |
| DR591 | | ACCGGCTCTACCAGCTATGCGGCCTCCGTCAAAGGACGCTTTACCGCATCCGAA |
| | | GATAAGGGCAAGAACATCGCTTACTTGCAGATGAACTCTCTGAAACCAGAGGAT |
| | | ACCGCTATGTACTATTGTAAGGCGTCATGCGTGCGTGGCCGGGCCGTGAGCGAA |
| | | TACTGGGGCCAAGGAACCCAAGTTACCGTCTCGTCAGGGGGGGGGTCCCAAGTG |
| | | CAGTTGCAAGAATCCGGTGGGGGATCTGTCCAAGCCGGAGGTTCCCTGAGACTC |
| | | TCATGTACCGCAAGCGGTGCCATTGCAAGCGGCTATATTGATTCCCGCTGGTGC |
| | | ATGGCTTGGTTCCGCCAGGCCCCAGGGAAGGAGCGTGAGGGTGTGGCCGCTATC |
| | | TGGCCCGGAGGGGGCTTGACCGTGTATGCCGATTCCGTCAAAGGACGTTTCACT |
| | | ATCAGCCGCGACCACGCCAAAAACACTCTTTATCTGCAAATGAATAACCTCAAG |
| | | CCAGAGGACACAGCTATGTACTATTGCGCCGCAGGCTCCCCACGCATGTGTCCT |
| | | TCCTTGGAGTTCGGCTTCGACTACTGGGGACAGGGTACTCAGGTTACTGTCTCC |
| | | TCC |
| hIL27 | 1268 | CAGGTGCAGCTCCAGGAGTCTGGAGGGGGTTCCGTTCAGGCCGGTGGCTCCCTG |
| Ra_VH | | CGTTTGTCTTGCGTGGCCAGTGGCTACGTCAGTTGTGACTACTTCCTGCCCTCA |
| H3- | | TGGTATCGTCAAGCGCCAGGTAAGGAGCGCGAGTTCGTGTCCATTATCGACGGA |
| DR591 | | ACAGGTTCTACTAGCTACGCCGCTTCTGTCAAGGGGCGCTTCACCGCTTCCGAG |
| | | GATAAGGGTAAGAACATCGCGTATCTGCAAATGAACTCATTGAAGCCCGAGGAC |
| | | ACCGCCATGTATTACTGTAAAGCCTCCTGTGTGCGTGGCCGCGCTGTGTCCGAG |
| | | TACTGGGGCCAGGGCACACAGGTGACCGTTTCCAGCGGCGGTTCCGGGGGGTCC |
| | | GGGGGCTCAGGGCAGGTGCAGCTTCAGGAGAGCGGAGGGGGATCTGTGCAGGCC |
| | | GGTGGCTCCCTGCGGGTGTCCTGCACAGCATCTGGGGCCATCGCGTCCGGGTAC |
| | | ATCGACTCCCGCTGGTGTATGGCCTGGTTCCGCCAAGCGCCTGGCAAGGAGCGT |
| | | GAGGGCGTGGCCGCGATCTGGCCGGGTGGCGGACTCACAGTCTACGCTGACAGC |
| | | GTCAAGGGGCGTTTCACTATTAGCAGAGACCACGCTAAGAACACCCTTTACCTT |
| | | CAGATGAATAACCTGAAGCCTGAAGACACCGCGATGTACTATTGCGCAGCTGGT |
| | | TCTCCCCGCATGTGCCCCTCACTTGAATTTGGTTTCGACTACTGGGGGCAGGGT |
| | | ACGCAGGTCACGGTGTCCTCC |
| hIL27 | 1269 | CAAGTCCAGCTCCAGGAGAGCGGTGGGGGGTCCGTGCAGGCGGGAGGCAGCCTC |
| Ra_VH | | CGCCTCAGTTGTGTCGCCTCTGGCTACGTGTCCTGCGATTATTTTCTTCCGTCC |
| H3- | | TGGTATCGCCAGGCTCCCGGCAAAGAAAGGGAGTTCGTTAGCATCATTGATGGC |
| DR592 | | ACGGGTTCCACAAGTTACGCTGCCAGCGTGAAAGGTAGGTTTACCGCTTCTGAA |
| | | GACAAAGGAAAGAACATCGCCTACTTGCAGATGAACTCTCTGAAGCCCGAGGAC |
| | | ACAGCCATGTACTATTGCAAAGCCTCTTGCGTGAGGGGACGGGGGGTGTCTGAA |
| | | TACTGGGGGCAAGGAACCCAAGTGACCGTGTCCTCTGGAGGCGGTAGCCAGGTC |
| | | CAGCTGCAAGAGTCCGGCGGAGGCTCTGTTCAGGCCGGTGGCTCACTGCGCCTG |
| | | TCTTGTACCGCACCTGGCTTCACTAGCAACCCTTGTGGAATGGACTGGTATCGT |
| | | CAGGCCCCCGGTAAGGAAAGAGAGTTCGTTTCTTCCATTTCTACAGACGGCACA |
| | | ACGGGGTATGCGGACAGTGTGAAAGGAAGATTCACTATCAGTAAGGATAAGGCC |
| | | AAAGACACCGTGTATCTCCAGATGAACTCCTTGAAACCAGAGGACACTGGAATG |
| | | TATAGCTGCAAGACCAAGGACGGGACCATTGCTACGATGGAACTCTGCGATTTC |
| | | GGCTACTGGGGGCAGGGGACACAGGTGACTGTGTCCTCG |
| hIL27 | 1270 | CAGGTGCAGTTGCAGGAGTCCGGGGCGGGGTCCGTGCAGGCTGGCGGTAGCCTG |
| Ra_VH | | CGCCTGAGTTGCGTGGCCTCTGGATACGTCTCTTGCGATTACTTCTTGCCCTCT |
| H3- | | TGGTATCGCCAGGCTCCCGGCAAGGAGCGCGAGTTCGTGAGCATCATTGACGGC |
| DR592- | | ACGGGTAGCACCAGCTACGCTGCCAGCGTGAAGGGACGTTTTACAGCCTCTGAG |
| | | GACAAGGGGAAGAACATTGCGTATCTTCAGATGAACTCCCTGAAGCCCGAGGAT |
| | | ACCGCCATGTACTATTGCAAGGCCTCCTGCGTGCGTGGCCGGGCCGTTAGTGAG |
| | | TATTGGGGCCAGGGGACGCAGGTCACCGTCAGTAGCGGTGGCTCCGGCGGTTCT |
| | | GGTGGCAGTGGTCAGGTCCAGCTCCAGGAGAGCGGTGGAGGCAGCGTCCAAGCC |
| | | GGCGGGTCCCTGAGGCTGTCTTGTACTGCGCCGGGATTTACTTCCAACAGCTGG |
| | | GGTATGGACTGGTATCGGCAGGCCCCTGGGAAAGAGAGGGAGTTCGTGTCTTCC |
| | | ATCTCTACGGATGGTACTACCGGCTACGCCGACAGCGTGAAGGGCCGTTTTACT |
| | | ATTAGCAAAGACAAGGCTAAAGACACAGTCTACCTTCAGATGAACTCTCTCAAA |
| | | CCAGAGGACACTGGTATGTATAGCTGTAAAACCAAAGATGGGACCATCGCTACA |
| | | ATGGAACTCTGCGATTTCGGGTACTGGGGACAGGGCACCCAAGTCACTGTCTCC |
| | | AGT |
| hIL27 | 1271 | CAGGTGCAGCTTCAGGAGTCCGGGGGAGGCAGCGTGCAGGCAGGAGGCAGCCTG |
| Ra_VH | | CGCCTGTCTTGCGTCGCTTCCGGCTACGTGTCCTGCGACTATTTCCTGCCTTCC |
| H3- | | TGGTACAGACAGGCACCTGGAAAGGAGCGCGAGTTCGTCTCTATTATCGACGGC |
| DR593- | | ACCGGCAGCACTTCTTACGCTGCCTCCGTTAAGGGCCGGTTCACCGCAAGCGAG |
| | | GACAAGGGAAAGAATATTGCCTACCTCCAGATGAACAGCCTGAAACCAGAGGAC |
| | | ACCGCGATGTATTACTGTAAGGCATCATGCGTGCGCGGTCGCGCAGTGTCAGAG |
| | | TATTGGGGTCAGGGAACCCAGGTGACAGTTTCTTCCGGGGGTGGCTCTCAGGTG |
| | | CAGTTGCAGGAGAGTGGTGGAGGGTCAGTCCAGGCTGGAGGCTCCCTCAGACTT |
| | | TCCTGTGCCGCTAGTGGATACCCCTATTCCAATGGATATATGGGTTGGTTTAGA |
| | | CAAGCTCCAGGAAAGGAGCGTGAGGGCGTCGCGACCATCTACACTGGCGACGGT |
| | | CGCACCTACTATGCGGATAGCGTGAAGGGCCGTTTCACCATCAGCCGTGACAAC |
| | | GCCAAGAATACCGTCGATTTGCAGATGTCTTCCTTGAAACCGGAGGACACCGCT |
| | | ATGTATTACTGTGCGGCCAGAGCTGCGCCATTGTATAGCTCCGGTTCCCCTCTG |
| | | ACCCGCGCTCGCTACAATGTGTGGGGACAGGGCACTCAGGTCACCGTCTCATCC |
| hIL27 | 1272 | CAGGTGCAACTCCAGGAGAGGGGGGGGGGTTCCGTCCAGGCTGGTGGCTCTCTG |
| Ra_VH | | AGGTTGAGCTGTGTAGCCAGCGGCTACGTGTCTTGTGACTACTTCCTCCCCAGC |
| H3- | | TGGTATCGTCAGGCTCCTGGCAAGGAGCGCGAGTTTGTCAGTATCATTGATGGC |
| DR593 | | ACCGGGAGCACCTCATACGCTGCCTCTGTGAAGGGTCGCTTCACTGCCAGCGAG |
| | | GATAAGGGCAAGAACATCGCCTACCTTCAGATGAACTCTTTGAAGCCGGAAGAC |
| | | ACTGCCATGTATTACTGCAAGGCTTCTTGCGTCAGGGGACGCGCAGTGTCAGAG |
| | | TATTGGGGGCAGGGAACCCAGGTTACCGTCAGCTCCGGGGGAAGCGGAGGCAGG |
| | | GGGGGCAGGGGGCAGGTGCAACTCCAGGAATCTGGGGGAGGCTCCGTTCAGGCT |
| | | GGCGGTTCTCTCAGACTCAGCTGCGCGGCCTCCGGCTATCCATATTCCAACGGA |
| | | TACATGGGCTGGTTCCGTCAGGCTCCCGGAAAGGAACGCGAAGGCGGGGCTACC |
| | | ATCTACACCGGAGATGGCCGGACTTATTACGCGGACTCTGTGAAGGGTCGCTTC |
| | | ACCATTTCAAGGGACAACGCAAAGAACACCGTGGACCTGCAAATGTCTTCCCTG |
| | | AAACCAGAAGACACCGCCATGTACTATTGTGCTGCCCGCGCCGCACCCCTGTAT |
| | | AGCTCCGGTTCCCCTGTTACGAGGGCACGTTACAACGTGTGGGGCCAGGGCACT |
| | | CAGGTGACAGTTAGCTCC |
| hIL27 | 1273 | CAAGTGCAGCTGCAAGAGAGTGGCGGAGGCTCTGTGCAGGCTGGCGGTTCCCTG |
| Ra_VH | | AGACTCAGTTGCGTTGCCTCAGGCTACGTGTCTTGTGACTACTTCTTGCCCTCC |
| H3- | | TGGTATCGGCAGGCTCCCGGAAAGGAGCGCGAGTTTGTTTCAATCATTGATGGG |
| DR594 | | ACAGGTTCTACCTCCTACGCCGCGTCAGTTAAGGGACGGTTTACTGCGTCCGAG |
| | | GACAAGGGAAAGAACATTGCATACCTTCAGATGAACTCTCTGAAGCCCGAAGAT |
| | | ACAGCCATGTATTACTGCAAGGCCTCGTGCGTCAGAGGTAGAGCAGTCTCCGAG |
| | | TACTGGGGCCAGGGAACCCAGGTGACAGTGAGTAGCGGAGGGGGCTCTCAGGTG |
| | | CAGCTGCAAGAGAGCGGCGGTGGCTCCGTTCAGGCCGGTGGGAGCCTCAGGCTC |
| | | AGCTGTGTGGCGAGCGCTTCCACTTATTGCACCTACGACATGCACTGGTATCGG |
| | | CAGGCTCCGGGAAAGGGCAGAGAGTTCGTATCTGCCATCGACTCAGACGGGACC |
| | | ACACGTTACGCCGACTCTGTGAAGGGCCGCTTCACTATCAGCCAAGGCACCGCC |
| | | AAAAACACTGTTTATTTGCAGATGAACAGCTTGCAGCCAGAGGACACCGCCATG |
| | | TATTACTGTAAGACAGTTTGTGTGGTAGGCAGCAGATGGTCCGACTACTGGGGC |
| | | CAGGGTACTCAAGTGACCGTCTCCAGC |
| hIL27 | 1274 | CAGGTCCAGCTCCAGGAAAGCGGCGGTGGCAGCGTGCAGGCTGGCGGATCTTTG |
| Ra_VH | | CGTCTTTCATGCGTGGCCAGCGGTTATGTAAGGTGCGACTATTTTCTGCCAAGC |
| H3- | | TGGTATCGGCAGGCACCTGGCAAGGAGCGTGAATTTGTCAGCATCATTGATGGC |
| DR594 | | ACCGGATCTACCTCCTACGCAGCGAGCGTCAAGGGAAGATTCACCGCCTCAGAA |
| | | GATAAGGGCAAAAACATCGCCTACCTCCAGATGAACTCCCTGAAGCCTGAAGAT |
| | | ACAGCGATGTACTATTGCAAAGCCAGCTGTGTCCGTGGCAGAGCCGTGTCCGAG |
| | | TATTGGGGCCAGGGAACACAAGTGACTGTTTCCTCTGGTGGATCAGGAGGCAGG |
| | | GGTGGCTCTGGCCAGGTGCAGGTTCAGGAGAGCGGAGGTGGATCTGTGCAGGCT |
| | | GGCGGTTCACTTCGTCTGTCCTGCGTCGCCTCTGCGTCTACCTATTGTACCTAC |
| | | GATATGCACTGGTATCGCCAAGCCCCTGGCAAGGGCCGCGAGTTCGTGTCCGCT |
| | | ATTGATTCCGACGGAACTACCCGGTACGCCGATTCTGTCAAGGGAAGATTTACA |
| | | ATCTCCCAGGGGACCGCCAAGAATACCGTCTATCTGCAAATGAACAGTTTGCAG |
| | | CCAGAGGACACTGCTATGTATTACTGCAAGACCGTGTGCGTGGTCGGAAGCCGC |
| | | TGGTCTGACTATTGGGGTCAGGGCACCCAGGTGACCGTGTCCTCG |
| hIL27 | 1275 | CAGGTCCAGCTCCAGGAGTCAGGTGGCGGTTCCGTCCAGGCCGGAGGCAGCCTG |
| Ra_VH | | CGGCTTTCCTGTGTGGCTTCTGGCTATGTTAGCTGCGACTACTTTCTCCCTTCC |
| H3- | | TGGTATCGTCAAGCGCCTGGAAAGGAGAGGGAGTTCGTCTCCATCATTGACGGG |
| DR595 | | ACGGGTTCCACCAGCTATGCAGCCTCCGTGAAGGGGAGATTCACTGCCAGTGAA |
| | | GACAAGGGTAAGAACATCGCTTATTTGCAGATGAACTCCCTGAAACCAGAGGAT |
| | | ACCGCTATGTATTACTGCAAGGCGTCTTGCGTGAGGGGCCGCGCTGTGTCCGAA |
| | | TACTGGGGCCAGGGGACCCAGGTCACCGTTAGGTCTGGAGGGGGTTCCCAGGTG |
| | | CAGCTTCAGGAAAGCGGAGGTGGCAGCGTGCAGGCTGGAGGCTCACTGACACTG |
| | | TCTTGCGCAGCCTCCGAATACGCATATTCAACCTGCAACATGGGCTGGTATCGT |
| | | CAGGCCCCTGGCAAGGAGAGAGAGCTGGTGTCTGCTTTCATCTCTGATGGCTCC |
| | | ACGTATTACGCTGATTCTGTTAAGGGTCGTTTTACCATCACACGGGACAACGCC |
| | | AAGAACACGGTGTATGTTCAGATGAACAGTTTGAAGCCCGAGGATACAGCGATC |
| | | TACTATTGCAGCGCGAATTGTTATAGACGCCTGCGCAACTATTGGGGTCAGGGC |
| | | ACACAGGTTACCGTAAGCTCA |
| hIL27 | 1276 | CAAGTTCAGGTGCAAGAATCTGGAGGCGGGTCTGTGCAGGCTGGGGGTTCCCTG |
| Ra_VH | | CGTCTCTCATGCGTCGCCTCTGGCTACGTTTCCTGCGACTACTTTCTTCCTTCA |
| H3- | | TGGTACAGGCAGGCCCCTGGCAAGGAGCGCGAGTTTGTGTCCATTATCGACGGG |
| DR595 | | ACTGGTAGCACATCCTATGCTGCCTCCGTGAAAGGACGGTTTACAGCCAGTGAG |
| | | GACAAGGGTAAGAACATCGCTTATCTGCAAATGAACTCACTCAAGCCCGAAGAC |
| | | ACAGCTATGTATTACTGCAAGGCATCCTGTGTGCGTGGCCGCGCCGTGTCTGAG |
| | | TACTGGGGGCAGGGGACACAGGTGACCGTGTCGTCTGGGCGGCTCAGGGGCTCC |
| | | GGTGGCTCCGGCCAAGTTCAGCTCCAGGAGTCTGGAGGCGGTAGCGTGCAGGCT |
| | | GGAGGCTCCCTGACCGTTAGCTGCGCCGCTAGTGAGTATGCGTATTCAACTTGC |
| | | AACATGGGTTGGTACAGGCAGGCCCCAGGGAAGGAGAGAGAGCTGGTCAGCGCT |
| | | TTCATTAGTGACGGGTCTACGTATTACGCCGACTCCGTGAAGGGGAGATTTACT |
| | | ATTACCCGTGATAACGCGAAGAATACTGTGTATTTGCAGATGAACTCTCTCAAG |
| | | CCGGAGGACACCGCCATCTATTACTGTAGCGCCAACTGTTACAGACGCCTGAGA |
| | | AACTATTGGGGGCAAGGAACCCAGGTGACCGTGAGCAGC |
| hIL27 | 1277 | CAGGTGCAACTGCAAGAGTCAGGGGGGGGCAGTGTGCAGGGCGGGGGCAGTCTG |
| Ra_VH | | CGCCTGTCCTGCGTAGCTTCTGGCTACGTGTCTTGCGACTACTTGCTGCCGTCC |
| H3- | | TGGTATAGGCAAGCGCCGGGGAAGGAACGGGAGTTTGTCAGTATCATTGATGGT |
| DR596 | | ACTGGTAGCACATCTTATGCTGCCTCTGTCAAGGGAAGATTTACTGCCAGCGAG |
| | | GATAAGGGCAAGAACATTGCCTACCTGCAAATGAACTCTCTGAAGCCAGAGGAT |
| | | ACCGCCATGTATTACTGCAAGGCCAGCTGCGTTAGGGGACGTGCTGTCTCTGAA |
| | | TATTGGGGCCAGGGCACCCAGGTCACCGTGAGTAGCGGTGGGGGGTCTCAGGTT |
| | | CAACTGCAAGAGTCTGGAGGTGGGCTCGTCCAGCCGGGGGGGTCTCTGAGGTTG |
| | | TCTTGCACCGCGTCAGGGCTGACCTTCGACGATTCTGTGATGGGCTGGTTTCGC |
| | | CAGGCCCCTGGCAAAGGGCGTGAGGCTGTGTCCTGCATCAGCTCAAGCGGAGCA |
| | | AATGCCTTCTATGCCGACAGTGTCAAAGGCCGGTTCACAATCAGCAGAGACAAC |
| | | GCTAAGAACACCCTGTACCTCCAGATGAACTCACTCAAGCCTGAAGATACGGCA |
| | | ACCTATTACTGCAAAAGGGGCCACGCCTGCGCCGGGTATTACCCTATCCCCTAT |
| | | GATGACTACTGGGGCCAGGGTACTCAAGTAACCGTGTCCTCC |
| hIL27 | 1278 | CAGGTCCAGGTTCAGGAGTCCGGCGGTGGCTCCGTGCAGGCTGGGGGGTCCCTG |
| Ra_VH | | CGCCTGTCCTGCGTGGCCAGCGGCTACGTGTCCTGCGACTATTTCCTGCCCTCT |
| H3- | | TGGTATCGCCAAGCGCCGGGCAAAGAAAGAGAGTTCGTATCAATCATTGATGGG |
| DR596 | | ACCGGCTCAACCAGCTACGCAGCTTCCGTAAAGGGGAGATTCACAGCCTCCGAA |
| | | GATAAAGGCAAGAACATCGCTTACCTCCAGATGAACTCTCTGAAGCCTGAGGAT |
| | | ACTGCAATGTACTATTGTAAGGCCTCATGCGTGCGTGGCCGCGCAGTGTCTGAA |
| | | TATTGGGGCCAGGGCACCCAGGTTACCGTCTCAAGCGGAGGCTCAGGAGGGTCA |
| | | GGAGGCTCAGGACAGGTGCAGCTTCAGGAGTCCGGCGGAGGCCTGGTGCAGCCC |
| | | GGCGGTAGCCTCCGTGTTAGTTGTACTGCCAGCGGGCTCACCTTTGATGACTCA |
| | | GTGATGGGGTGGTTTCGCCAGGCTCCGGGCAAAGGCCGGGAGGCCGTGTCCTGC |
| | | ATCTCTAGCTCCGGTGCCAATGCGTTCTACGCAGATAGCGTGAAGGGCAGATTC |
| | | ACTATCAGTAGAGATAATGCCAAGAATACACTGTACTTGCAGATGAACTCCCTC |
| | | AAGCCTGAAGACACGGCCACCTACTATTGTAAACGGGGCCCACGCCTGCGTGGC |
| | | TATTACCCGATCCCGTATGATGACTACTGGGGACAGGGCACCCAAGTCACCGTC |
| | | TCCTGT |
| hIL27 | 1279 | CAGGTTCAGTTGCAGGAATCTGGTGGGGGCCTCGTTCAGCCTGGGGAGAGCCTC |
| Ra_VH | | CGTCTGTCCTGCACCGGTTCAGGATTCACTTTCTCAAACTACGCAATGTCTTGG |
| H4- | | GTCCGGCAGGCTCCGGGCAAAGGCTTGGAATGGGTGTCAGGCATCAATGTTGCA |
| DR591 | | TACGGCATTACCTCCTACGCAGATAGCGTGAAGGGCCGTTTCACCATCAGTCGT |
| | | GACAACACCAAAAACACTCTCTACTTGCAGCTCAATTCACTCAAGACAGAGGAC |
| | | ACCGCTATTTATTACTGTGTCAAACACTCCGGCACTACCATCCCACGCGGCTTC |
| | | ATTAGCTATACCAAACGCGGCCAGGGCACCCAGGTCACCGTCTCCTCTGGTGGC |
| | | GGTTCCCAGGTGCAGTTGCAGGAATCCGGGGGGGGAAGCGTGCAGGCCGGTGGC |
| | | AGCCTGCGCCTGAGCTGCACAGCTTCCGGGGCTATCGCTTCTGGCTACATTGAT |
| | | TCCCGGTGGTGTATGGCCTGGTTCCGCCAGGCCCCTGGCAAAGAGCGTGAGGGC |
| | | GTGGCTGCGATCTGGCCTGGAGGGGGCCTGACCGTCTACGCCGATAGCGTTAAA |
| | | GGCAGGTTTACCATCAGCCGCGACCATGCCAAGAACACCCTGTACCTCCAGATG |
| | | AATAACCTGAAGCCTGAGGACACGGCCATGTATTACTGCGCCGCAGGCTCACCT |
| | | AGAATGTGCCCTAGTTTGGAGTTCGGCTTTGACTATTGGGGTCAGGGTACGCAG |
| | | GTGACTGTGTCCTCC |
| hIL27 | 1280 | CAAGTGCAGTTGCAGGAGTCCGGTGGCGGACTCGTCCAGCCTGGAGAATCCCTG |
| Ra_VH | | AGGTTGAGCTGCACCGCGTCCGGGTTCACGTTCAGCAATTACGCCATGTCTTGG |
| H4- | | GTGAGGCAAGCCCCAGGTAAAGGCCTTGAGTGGGTCAGCGGAATCAACGTGGCT |
| DR591 | | TACGGGATCACCAGTTACGCAGATAGCGTCAAGGGGCGGTTCACTATTAGCAGA |
| | | GATAACACTAAGAACACTCTGTACCTTCAGCTGAACTCACTCAAGACCGAAGAC |
| | | ACCGCCATTTATTACTGCGTAAAGCACAGCGGTACTACGATCCCTCGCGGCTTT |
| | | ATCAGCTATACTAAGCGCGGTCAAGGAACCCAAGTGACCGTGTCAAGCGGAGGG |
| | | AGCGGTGGATCTGGGGGATCAGGCCAGGTGCAGCTTCAGGAGAGTGGCGGTGGC |
| | | TCTGTGCAGGCCGGGGGCTCACTGAGACTGTCTTGCACTGCCAGGGGGGCTATC |
| | | GCGTCTGGCTACATCGACAGTCGCTGGTGCATGGCCTGGTTCAGACAGGCCCCT |
| | | GGTAAGGAGCGTGAAGGCGTGGCTGCCATTTGGCCTGGAGGCGGACTGACCGTC |
| | | TACGCGGATAGCGTCAAGGGGAGGTTTACCATTAGCCGCGACCACGCCAAGAAT |
| | | ACTCTGTACCTTCAGATGAATAACCTGAAACCCGAGGATACCGCGATGTATTAC |
| | | TGTGCAGCTGGCAGTCCTCGGATGTGTCCGAGCTTGGAGTTCCGGGTTCGATTA |
| | | TGGGGTCAGGGAACTCAGGTGACTGTGTCCTCG |
| hIL27 | 1281 | CAGGTACAGCTCCAGGAATCCGGCGGAGGCCTTGTCCAGCCCGGAGAGTCCTTG |
| Ra_VH | | CGCCTCTCTTGCACCGCAAGCGGCTTCACCTTGTCCAACTATGCTATGTCTTGG |
| H4- | | GTGCGTCAGGCCCCCGGCAAAGGCCTGGAATGGGTCAGCGGGATCAATGTGGCA |
| DR592 | | TACGGGATCACTTCCTACGCCGATAGTGTCAAGGGTCGTTTTACCATTAGCCGT |
| | | GATAACACGAAAAACACTCTGTACCTCCAGCTCAACAGCCTGAAGACGGAAGAT |
| | | ACGGCTATCTATTACTGTGTCAAACACAGTGGCACGACTATTCCGCGCGGCTTC |
| | | ATCTCATATACTAAGCGCGGACAGGGGACCCAAGTGACTGTCAGCAGTGGCGGA |
| | | GGCTCCCAGGTGCAGCTCCAGGAATCCGGTGGGGGTTCCGTCCAAGCCGGGGGG |
| | | TCTCTGCGTCTTTCCTGTACCGCTCCGGGGTTTACCAGCAACTCTTGTGGAATG |
| | | GACTGGTACAGGCAGGCACCAGGGAAGGAGCGCGAGTTCGTTTCCAGCATCAGC |
| | | ACCGACGGAACCACTGGCTATGCCGACAGCGTGAAGGGTCGCTTCACGATCTCC |
| | | AAGGATAAGGCCAAAGATACTGTGTACCTGCAAATGAACTCTCTTAAACCGGAA |
| | | GATACAGGTATGTACTCATGCAAAACAAAAGACGGCACCATCGCCACGATGGAG |
| | | CTTTGCGATTTCGGATACTGGGGCCAGGGCACACAAGTGACGGTTAGTAGC |
| hIL27 | 1282 | CAGGTCCAGGTCCAGGAGAGCGGCGGGGGCCTGGTCCAGCCAGGTGAGTCTCTG |
| Ra_VH | | CGTCTGTCTTGCACCGCTTCTGGTTTCACATTTTCCAATTATGCCATGTCCTGG |
| H4- | | GTCCGCCAGGCTCCAGGGAAGGGCCTTGAGTGGGTCTCTGGCATCAACGTGGCC |
| DR592 | | TATGGCATCACCTCTTATGCCGACAGCGTGAAGGGTCGTTTCACCATCTCCCGC |
| | | GACAACACGAAGAACACACCGTACCTCCAGTTGAACAGGTTGAAGACCGAGGAC |
| | | ACCGCGATCTACTATTGCGTGAAGCATAGGGGACCACGATCCCTCCGCGGATTG |
| | | ATCAGCTATACGAAGCGCGGCCAGGGTACACAGGTCACCGTTAGGTCCGGGGGT |
| | | AGTGGGGGGTCCGGTGGCAGCGGACAGGTGCAGCTCCAGGAGTCCGGCGGGGGT |
| | | TCCGTACAAGCCGGAGGCTCACTGCGCCTGTCCTGCACAGCTCCGGGTTTCACT |
| | | TCTAACAGTTGCGGCATGGACTGGTATCGCCAAGCCCCAGGCAAAGAAAGAGAG |
| | | TTCGTTTCTAGCATCTCCACCGATGGCACCACAGGTTACGCCGACTCTGTCAAG |
| | | GGCCGCTTTACTATCAGCAAGGATAAGGCCAAGGACACCGTGTATGTTCAGATG |
| | | AACTCCCTGAAGCCCGAGGATACTGGGATGTACTCATGCAAGACCAAGGATGGA |
| | | ACTATCGCCACGATGGAGCTGTGTGACTTCGGTTATTGGGGGCAGGGCACCCAG |
| | | GTGACCGTCAGCAGC |
| hIL27 | 1283 | CAGGTGCAGCTCCAGGAGAGTGGCGGGGGCCTGGTTCAGCCAGGTGAAAGCCTG |
| Ra_VH | | CGCTTGAGCTGCACCGCATCCGGCTTCACCTTCTCTAATTATGCCATGAGTTGG |
| H4- | | GTGAGACAAGCCCCCGGCAAAGGACTGGAGTGGGTGTCTGGAATTAACGTAGCG |
| DR593 | | TATGGCATCACGTCCTACGCTGATAGCGTCAAGGGGCGCTTCACCATCTCCCGC |
| | | GACAACACGAAAAACACCCTGTACCTCCAGCTGAACTCCCTCAAGACTGAAGAT |
| | | ACCGCGATTTATTACTGTGTGAAACACTCTGGAACAACCATCCCTCGCGGCTTC |
| | | ATTAGTTACACCAAGCGCGGCCAGGGCACCCAGGTGACCGTCAGTTCGGGGGGC |
| | | GGAAGCCAGGTGCAGTTGCAGGAGTCCGGCGGAGGTTCCGTGCAGGCTGGGGGG |
| | | TCCCTGAGACTGTCCTGCGCAGCCAGCGGCTACCCTTATAGCAATGGCTACATG |
| | | GGATGGTTTCGCCAGGCTCCTGGCAAGGAGCGCGAAGGTGTGGCCACTATTTAC |
| | | ACGGGTGATGGACGCACCTATTACGCCGACAGCGTCAAGGGCCGGTTTACCATT |
| | | AGCCGGGATAACGCCAAGAATACTGTCGATCTCCAGATGTCTTCACTGAAACCT |
| | | GAAGACACCGCCATGTACTATTGCGCCGCACGCGCCGCGCCGTTGTACTCTTCC |
| | | GGCAGCCCCCTGACCCGTGCTCGCTACAACGTCTGGGGGCAAGGCACACAGGTG |
| | | ACAGTCTCCTCT |
| hIL27 | 1284 | CAGGTCCAGTTGCAGGAGAGCGGAGGGGGCCTGGTGCAGCCTGGGGAATCCCTC |
| Ra_VH | | CGTCTCTCCTGCACCGCCAGCGGCTTCACCTTCAGCAACTACGCCATGAGCTGG |
| H4- | | GTGCGCCAGGCTCCAGGGAAGGGCCTGGAGTGGGTGTCAGGCATTAACGTCGCC |
| DR593 | | TACGGCATCACCAGTTATGCCGATAGTGTCAAGGGGCGGTTCACTATCTCCAGG |
| | | GACAACACAAAGAACACTCTCTATCTTCAGCTTAATTCCCTGAAAACTGAAGAC |
| | | ACCGCTATCTATTACTGTGTGAAACACAGCGGAACAACCATCCCGCGCGGTTTC |
| | | ATCTCCTACACTAAGAGAGGCCAGGGCACCCAGGTTACCGTTTCTAGCGGCGGA |
| | | TCTGGGGGCAGCGGGGGCTCCGGTCAGGTCCAACTCCAAGAGTCTGGCGGAGGG |
| | | TCTGTCCAGGCTGGCGGGTCTTTGCGTCTGTCCTGTGCCGGTTCTGGCTACCCG |
| | | TACAGCAACGGTTACATGGGTTGGTTCAGACAGGCCCCCGGCAAAGAGAGGGAG |
| | | GGAGTGGCCACCATCTACACCGGCGACGGCAGGACATATTACGCTGATTCCGTG |
| | | AAGGGTCGTTTCACTATCTCCCGCGATAACGCCAAGAACACCGTCGATCTTCAG |
| | | ATGTCATCCCTGAAGCCTGAAGACACTGCGATGTATTACTGTGCAGCTCGCGCC |
| | | GCGCCCCTCTACAGCTCAGGTAGCCCACTGACACGTGCCCGGTACAACGTCTGG |
| | | GGGCAGGGCACCCAGGTGACGGTGTCTTCC |
| hIL27 | 1285 | CAGGTGCAGCTCCAGGAAAGCGGTGGCGGGCTGGTTCAGCCTGGCGAGTCCCTC |
| Ra_VH | | CGTCTGTCCTGCACAGCTAGTGGTTTTACGTTTAGCAACTACGCGATGAGCTGG |
| H4- | | GTCCGTCAAGCCCCTGGTAAGGGGCTGGAGTGGGTGTCCGGCATCAACGTGGCC |
| DR594 | | TACGGCATCACCTCCTACGCTGACTCTGTGAAGGGCCGGTTCACTATCAGCCGG |
| | | GATAACACTAAGAACACCTTGTATCTCCAACTGAACTCCCTGAAGACAGAGGAC |
| | | ACCGCTATTTACTATTGTGTGAAGCACTCTGGCACGACCATCCCCAGGGGCTTC |
| | | ATCAGCTACACCAAGCGTGGGCAGGGTACACAGGTGACAGTCTCCAGCGGCGGA |
| | | GGCTCCCAAGTGCAGCTGCAAGAATCTGGGGGGGGAAGCGTCCAGGCCGGTGGA |
| | | AGTCTGCGCCTTAGTTGCGTGGCTTCTGCAAGCACCTATTGCACCTATGACATG |
| | | CACTGGTATAGGCAGGCCCCCGGCAAGGGGCGCGAGTTCGTTAGCGCCATCGAC |
| | | AGTGATGGGACCACGCGCTACGCGGACAGTGTGAAGGGCAGGTTCACCATCAGC |
| | | CAGGGTACTGCCAAAAATACAGTGTACTTGCAGATGAACTCCCCCACGCCTGAG |
| | | GACACCGCCATGTACTATTGCAAGACCGTGTGCGTCGTAGGCTCCCGTTGGAGC |
| | | GACTACTGGGGCCAGGGCACACAGGTGACCGTGTCCTCC |
| hIL27 | 1286 | CAGGTCCAACTGCAAGAGTCTGGAGGTGGATTGGTCCAGCCCGGCGAAAGCCTG |
| Ra_VH | | AGGTTGAGCTGCACCGCGAGCGGATTCACCTTTAGCAACTATGCCATGTCTTGG |
| H4- | | GTTCGCCAAGCCCCTGGGAAGGGCCTGGAGTGGGTCAGTGGGATCAATGTGGCT |
| DR594 | | TATGGAATCACCAGCTATGCGGACTCTGTGAAGGGAAGGTTCACTATCTCTCGC |
| | | GACAATACTAAAAACACCCTGTACCTCCAGCTCAATTCCCTCAAGACTGAGGAC |
| | | ACCGCTATTTACTATTGTGTGAAACACTCCGGCACCACGATCCCCCGTGGGTTC |
| | | ATCAGCTACACCAAACGGGGCCAAGGCACCCAGGTCACCGTATCTAGTGGCGGT |
| | | AGTGGTGGGAGTGGGGGTTCCGGGCAGGTGCAGCTCCAGGAGTCCGGCGGTGGC |
| | | TCCGTGCAGGCAGGGGGTAGCCTGCGCCTGTCCTGTGTGGCGTCTGCAAGCACC |
| | | TATTGTACTTATGATATGCACTGGTATCGTCAGGCTCCAGGCAAGGGCAGAGAG |
| | | TTCGTCTCCGCAATCGACTCCGATGGCACCACGCGCTACGCCGATTCTGTGAAG |
| | | GGCCGTTTCACTATCAGCCAGGGTACAGCCAAAAATACCGTGTATCTTCAGATG |
| | | AACAGTCTCCAGCCAGAGGATACCGCTATGTACTATTGCAAGACTGTGTGCGTG |
| | | GTTGGCTCCAGGTGGTCCGATTACTGGGGCCAGGGAACCCAGGTGACAGTGTCC |
| | | AGC |
| hIL27 | 1287 | CAGGTGCAGCTCCAGGAGAGCGGAGGGGGTTTGGTCCAGCCTGGCGAGTCTCTC |
| Ra_VH | | AGACTTTCTTGTACCGCATCTGGCTTTACTTTCAGCAATTACGCCATGTCCTGG |
| H4- | | GTGCGGCAAGCCCCCGGCAAGGGCCTGGAATGGGTGAGCGGCATCAACGTGGCC |
| DR595 | | TACGGCATCACCTCATACGCAGATTCCGTCAAGGGGCGTTTTACCATCAGTCGC |
| | | GATAATACAAAGAACACATTGTACCCCCAACTGAACTCCCTCAAGACCGAGGAT |
| | | ACCGCGATTTACTATTGCGTGAAACACAGCGGCACTACCATCCCACGCGGGTTT |
| | | ATCAGCTACACGAAGAGAGGCCAGGGTACACAGGTCACCGTGTCAAGGGGCGGT |
| | | GGATCTCAGGTACAGGTCCAGGAGTCTGGGGGTGGAAGCGTGCAGGCGGGGGGA |
| | | TCTCTGACATTGTCCTGCGCCGCAAGTGAGTATGCTTATTCCACGTGCAACATG |
| | | GGATGGTACAGGCAGGCCCCTGGTAAAGAGAGAGAACTCGTGAGCGCTTTTATT |
| | | TCAGACGGCAGCACTTATTACGCCGACTCCGTCAAAGGACGCTTCACCATCACT |
| | | CGTGACAACGCTAAGAATACGGTCTACCTTCAGATGAACTCCCTCAAACCTGAA |
| | | GATACTGCAATCTACTATTGTAGTGCCAACTGCTATAGACGCCTCCGCAACTAT |
| | | TGGGGGCAGGGAACACAGGTGACCGTTAGCAGG |
| hIL27 | 1288 | CAAGTTCAGCTGCAAGAGTCTGGCGGTGGCCTGGTGCAGCCCGGCGAATCTTTG |
| Ra_VH | | CGCTTGAGTTGTACCGCCTCTGGTTTTACATTGTCAAACTATGCAATGAGCTGG |
| H4- | | GTGAGACAGGCCCCAGGCAAGGGCTTGGAATGGGTCTCTGGGATCAACGTCGCT |
| DR595 | | TACGGCATTACCTCATACGCCGATAGCGTCAAGGGTCGGTTCACTATCTCTCGG |
| | | GATAATACGAAGAATACTCTCTACTTGCAGCTCAACTCACTGAAGACCGAGGAC |
| | | ACTGCGATCTATTACTGCGTCAAACACTCCGGCACCACTATCCCTCGCGGCTTC |
| | | ATCTCTTACACTAAACGCGGCCAGGGTACTCAGGTGACTGTGAGTTCAGGTGGC |
| | | TCCGGAGGTAGCGGGGGTTCCGGTCAGGTGCAGCTCCAGGAGAGCGGGGGCGGA |
| | | AGCGTACAGGCTGGAGGCAGCCTGACCTTGAGCTGTGCAGCTTCCGAGTATGCC |
| | | TACTCCACCTGTAACATGGGATGGTATAGACAGGCTCCCGGCAAGGAACGTGAG |
| | | CTGGTCAGCGCCTTTATCAGTGATGGGTCCACTTACTATGCCGATTCCGTGAAA |
| | | GGCCGGTTCACCATCACTCGCGACAACGCCAAGAATACAGTGTACTTGCAGATG |
| | | AACTCCCTCAAGCCCGAGGACACTGCTATCTATTACTGTTCTGCAAACTGCTAC |
| | | CGTCGCCTGCGCAACTACTGGGGCCAAGGCACTCAGGTTACCGTGAGCAGC |
| hIL27 | 1289 | CAGGTGCAGTTGCAGGAGTCTGGAGGTGGCCTGGTCCAACCTGGAGAATCCCTG |
| Ra_VH | | CGGCTCTCTTGTACCGCGAGTGGTTTCACCTTCAGCAACTATGCAATGAGCTGG |
| H4- | | GTGCGCCAGGCTCCCGGCAAGGGATTGGAGTGGGTCAGGGGCATCAACGTGGCC |
| DR596 | | TATGGCATCACCAGCTACGCTGATAGTGTGAAGGGCCGGTTTACCATCAGTAGG |
| | | GACAACACCAAGAACACACCGTATCTTCAGCTCAACTCCCTGAAGACAGAAGAC |
| | | ACCGCTATCTACTATTGTGTGAAGCATTCCGGCACTACCATCCCACGCGGGTTC |
| | | ATCTCATACACCAAAAGGGGCCAGGGCACCCAGGTGACAGTTTCTAGGGGTGGC |
| | | GGTTCCCAAGTGCAGTTGCAGGAGTCCGGTGGCGGTCTGGTTCAGCCTGGTGGG |
| | | TCTCTTCGTCTGAGTTGTACCGCAAGCGGTCTTACCTTCGATGACAGCGTCATG |
| | | GGGTGGTTCAGGCAGGCCCCCGGCAAGGGTCGCGAGGCAGTGAGCTGCATCAGG |
| | | TCCAGTGGGGCTAACGCCTTTTATGCAGACCCCGTAAAAGGAAGATTCACTATC |
| | | AGCAGGGACAACGCAAAGAACACCCTGTATCTCCAAATGAACAGTCTGAAACCG |
| | | GAAGACACCGCCACTTATTACTGTAAACGCGGCCACGCCTGCGCAGGGTATTAC |
| | | CCGATTCCTTATGATGACTACTGGGGCCAAGGCACCCAGGTGACAGTGTCTAGC |
| hIL27 | 1290 | CAGGTGCAGCTCCAGGAAAGCGGAGGGGGGCTGGTGCAGCCGGGAGAGAGTCTC |
| Ra_VH | | AGGCTTTCTTGTACCGCCTCTGGATTTACATTCAGCAATTACGCCATGTCCTGG |
| H4- | | GTTCGCCAAGCACCAGGGAAGGGCCTGGAGTGGGTGTCCGGCATCAACGTGGCA |
| DR596 | | TACGGCATCACATCCTACGCGGATTCCGTCAAGGGTCGGTTTACCATCAGCCGG |
| | | GATAATACAAAGAACACCTTGTATCTCCAGCTTAACAGCCTCAAGACAGAGGAC |
| | | ACCGCCATCTACTATTGTGTGAAGCACTCCGGCACCACTATCCCTCGCGGTTTC |
| | | ATTTCCTACACCAAAAGGGGTCAGGGCACTCAGGTAACAGTGTCCTCTGGGGGC |
| | | TCCGGTGGATCAGGTGGCTCCGGCCAGGTACAACTCCAAGAGTCAGGAGGCGGT |
| | | CTGGTACAGCCTGGTGGCTCCTTGCGCCTGTCTTGCACAGCAAGCGGCCTGACT |
| | | TTCGATGACTCTGTGATGGGCTGGTTTCGTCAGGCCCCAGGGAAGGGCAGGGAG |
| | | GCCGTCTCCTGCATTAGTAGCTCCGGCGCTAACGCCTTCTATGCCGATAGTGTG |
| | | AAGGGGCGCTTTACCATCTCTCGTGACAATGCCAAGAACACCCTGTACTTGCAG |
| | | ATGAACAGCCTGAAGCCGGAGGATACTGCCACCTATTACTGCAAGCGCGGCCAT |
| | | GCGTGTGCTGGCTACTATCCCATCCCTTACGATGACTATTGGGGTCAGGGCACC |
| | | CAGGTGACCGTGTCCAGC |
| hIL27 | 1291 | CAGGTCCAGCTTCAGGAAAGCGGGGGTGGATCTGTCCAGGGGGGCGGATCACTT |
| Ra_VH | | CGCCTCAGTTGTACGGCCAGTGGCTATGTATCCTGCGACTACTTCCTCCCATCA |
| H5- | | TGGTATCGCCAGGCTCCGGGAAAGGAAAGGGAGTTCGTGTCTGTGATCGACGGC |
| DR591 | | ACTGGCTCCACCTCCTACGCTGCCAGCGTCAAGGGTCGCTTCACCGCCTCACAG |
| | | GACAAGGGCAAAAACATCGCCTATCCTCAGATGAACTCCCTGAAACCCGAGGAC |
| | | ACAGCCATGTATTACTGTAAGGCCTCCTGTGTGCGTGGCCGCGCAATTTCCGAA |
| | | TATTGGGGCCAGGGCACTCAGGTGACTGTGAGTTCCGGCGGGGGTTCCCAGGTC |
| | | CAGTTGCAGGAGTCCGGTGGCGGATCTGTGCAGGCCGGAGGGTCCCTGCGCCTG |
| | | AGCTGCACAGCGAGCGGTGCCATCGCCTCCGGCTATATTGACTCCAGGTGGTGC |
| | | ATGGCCTGGTTCAGGCAAGCGCCCGGAAAAGAGCGTGAAGGTGTAGCTGCCATC |
| | | TGGCCTGGCGGAGGCCTGACCGTGTATGCCGATTCCGTCAAGGGCCGCTTCACC |
| | | ATCAGCCGCGACCACGCAAAAAACACTCTGTACCTCCAGATGAATAACCTGAAG |
| | | CCGGAGGACACCGCCATGTATTACTGTGCTGCCGGTTCTCCCCGTATGTGCCCC |
| | | AGCCTGGAGTTCGGCTTCGACTACTGGGGCCAGGGAACTCAGGTCACTGTGTCA |
| | | TCT |
| hIL27 | 1292 | CAGGTCCAGCTCCAAGAGTCTGGAGGTGGCTCTGTGCAGGCTGGCGGTTCCCTC |
| Ra_VH | | CGCCTGAGCTGCACAGCGAGGGGGTACGTGAGCTGCGATTATTTTCTGCCGTCC |
| H5- | | TGGTATCGTCAAGCGCCCGGAAAGGAGCGCGAGTTCGTGTCCGTGATCGACGGT |
| DR591 | | ACGGGTAGCACCAGTTACGCGGCTTCTGTAAAAGGACGGTTCACCGCTTCCCAG |
| | | GATAAGGGCAAAAACATCGCGTATTTGCAGATGAACAGCCTGAAGCCCGAGGAT |
| | | ACCGCCATGTATTACTGTAAGGCCAGTTGTGTCAGAGGCCGCGCCATCTCCGAG |
| | | TACTGGGGACAGGGCACTCAGGTGACCGTGTCCAGCGGTGGAAGCGGTGGCTCC |
| | | GGGGGCTCCGGTCAGGTCCAGTTGCAAGAGTCAGGCGGTGGCTCCGTCCAGGCG |
| | | GGCGGGAGCCTGCGCTTGTCCTGCACAGCTTCTGGGGCCATCGCGTCCGGCTAT |
| | | ATCGACAGCCGCTGGTGCATGGCGTGGTTCCGCCAAGGGCCGGGCAAGGAACGT |
| | | GAAGGCGTCGCAGCTATTTGGCCTGGGGGGGGGTTGACCGTGTACGCCGACTCC |
| | | GTGAAGGGCCGTTTCACCATCAGTAGAGACCACGCCAAGAACACTCTCTACCTC |
| | | CAGATGAATAACCTGAAACCGGAGGACACTGCTATGTATTACCGCGCTGCGGGG |
| | | TCCCCCAGGATGTGTCCGAGCCTGGAGTTCGGCTTCGACTATTGGGGGCAGGGG |
| | | ACCCAGGTCACCGTGTCCTCC |
| hIL27 | 1293 | CAGGTTCAGGTCCAGGAATCCGGCGGAGGCTCTGTCCAGGCTGGGGGTTCTTTG |
| Ra_VH | | CGTTTGAGCTGTACCGCGAGCGGGTACGTGTCTTGCGACTACTTCTTGCCTTCC |
| H5- | | TGGTATCGCCAGGCTCCTGGTAAGGAACGCGAGTTCGTCTCCGTCATTGATGGC |
| DR592 | | ACCGGCTCTACGAGCTATGCTGCCTCAGTTAAGGGCCGGTTCACAGCCAGCCAG |
| | | GATAAGGGCAAAAATATCGCTTACCTCCAGATGAACTCTCTGAAACCTGAAGAC |
| | | ACTGCCATGTATTACTGTAAAGCGAGCTGCGTAAGAGGACGTGCCATCAGCGAG |
| | | TACTGGGGCCAAGGGACACAGGTGACAGTTAGTAGCGGTGGGGGGTCCCAGGTG |
| | | CAGTTGCAAGAGAGCGGTGGCGGAAGCGTGCAGGCAGGCGGATCTCTGCGGCTG |
| | | TCCTGCACCGCCCCTGGATTCACCAGTAACTCTTGTGGTATGGACTGGTATCGT |
| | | CAGGGTCCGGGTAAGGAGAGAGAGTTTGTTTCAAGTATGTCCACTGATGGCACA |
| | | ACCGGGTATGCCGACTCCGTCAAGGGTCGTTTCACCATCTCCAAGGACAAGGCC |
| | | AAGGACACTGTGTACCTTCAGATGAACTCACTGAAGCCTGAGGACACCGGGATG |
| | | TATTCTTGCAAGACTAAAGATGGCACCATTGCCACTATGGAGCTGTGCGACTTC |
| | | GGCTATTGGGGTCAGGGGACACAGGTCACTGTATCTAGC |
| hIL27 | 1294 | CAGGTGCAGCTTCAGGAGTCCGGCGGTGGGAGCGTTCAGGCTGGAGGCTCTCTC |
| Ra_VH | | AGGCTGTCATGCACTGCAAGCGGGTACGTTAGTTGTGACTACTTTCTTCCAAGC |
| H5- | | TGGTATCGCCAGGCTCCCGGAAAAGAGCGCGAGTTTGTTTCCGTTATTGATGGA |
| DR592 | | ACTGGGAGTACGTCCTATGCCGCTTCAGTCAAGGGGCGCTTCACCGCATCCCAG |
| | | GACAAGGGTAAGAATATCGCTTACCTTCAGATGAACTCCCTGAAGCCCGAAGAC |
| | | ACCGCGATGTATTACTGTAAGGCTTCTTGCGTGAGGGGCCGCGCTATCAGTGAA |
| | | TACTGGGGTCAGGGGACCCAGGTGACGGTTTCTAGCGGAGGCAGGGGGGGGAGC |
| | | GGCGGGTCTGGACAGGTCCAGCTTCAGGAAAGTGGTGGGGGCTCCGTTCAGGCT |
| | | GGCGGATCACTGCGCGTGAGCTGTACTGCTCCCGGCTTCACGAGCAACTCCTGC |
| | | GGTATGGACTGGTATCGTCAGGCCCCAGGTAAGGAGCGTGAGTTCGTGTCTTCC |
| | | ATCAGTACAGACGGGACAACCGGCTACGCAGACAGCGTAAAGGGCCGCTTCACC |
| | | ATCTCCAAGGATAAGGCAAAGGACACCGTCTACCTCCAGATGAACAGCCTGAAG |
| | | CCAGAGGACACGGGTATGTATAGCTGTAAGACCAAAGACGGTACAATCGCTACA |
| | | ATGGAGCTGTGTGACTTCGGGTATTGGGGCCAGGGAACTCAGGTTACCGTCTCA |
| | | TCC |
| hIL27 | 1295 | CAGGTGCAGCTCCAGGAGAGCGGGGGGGGTTCTGTGCAAGCTGGGGGAAGTCTG |
| Ra_VH | | CGGCTCAGCTGTACCGCTAGTGGTTACGTGTCTTGCGACTATTTCCTGCCTTCA |
| H5- | | TGGTATCGCCAGGCCCCAGGCAAAGAGGGCGAGTTCGTGTCCGTCATTGACGGA |
| DR593 | | ACCGGGAGTACCTCCTATGCAGCCAGTGTTAAGGGCCGTTTTACAGCCAGCCAG |
| | | GACAAGGGCAAAAATATCGCTTACCTCCAGATGAACAGCCTGAAGCCCGAGGAC |
| | | ACTGCCATGTATTACTGTAAAGCGAGCTGTGTCAGGGGTAGAGCAATTAGTGAA |
| | | TATTGGGGCCAAGGGACACAGGTGACAGTATCTTCAGGGGGGGGATCTCAGGTG |
| | | CAGCTCCAGGAAAGTGGAGGTGGGTCCGTGCAGGCGGGGGGTTCTCTCAGACTG |
| | | TCTTGCGCCGCGTCTGGCTATCCTTACAGCAACGGGTACATGGGATGGTTTAGA |
| | | CAAGCCCCAGGGAAGGAGCGCGAGGGGGTGGCCACCATTTACACCGGCGATGGC |
| | | CGGACTTACTATGCGGATTCCGTGAAGGGCCGGTTTACCATTTCAAGAGATAAC |
| | | GCGAAAAATACCGTGGACCTCCAGATGTCCTCTTTGAAGCCAGAGGATACCGCG |
| | | ATGTACTATTGCGCTGCCCGCGCCGCTCCTCTCTACAGTAGCGGGTCCCCACTT |
| | | ACCCGCGCTCGCTATAACGTGTGGGGCCAGGGGACACAGGTGACTGTGTCCTCC |
| hIL27 | 1296 | CAGGTGCAGCTTCAGGAATCTGGGGGTGGGTCCGTCCAGGCTGGCGGATGTTTG |
| Ra_VH | | AGACTGTCTTGCACTGCCAGTGGTTACGTGAGCTGTGATTACTTCCTGCCGTCC |
| H5- | | TGGTATCGTCAAGCGCCAGGCAAGGAACGTGAGTTCGTGTCCGTGATCGACGGG |
| DR593 | | ACAGGTTCCACATCTTACGCCGCATCTGTTAAGGGGAGGTTCACAGCTTCTCAG |
| | | GACAAGGGTAAAAATATCGCCTATCTCCAGATGAACTCACTGAAGCCTGAAGAT |
| | | ACCGCTATGTATTACTGCAAGGCTTCTTGTGTGGGGGGCCGCGCTATCAGCGAG |
| | | TACTGGGGACAGGGCACTCAGGTGACGGTCTCCAGGGGGGGGAGCGGGGGCTCC |
| | | GGGGGCAGCGGCCAGGTGCAGTTGCAGGAGAGCGGGGGAGGCCCTGTGCAAGCC |
| | | GGAGGCTCTTTGCGCCTGTCCTGCGCTGCCAGCGGATATCCTTACTCTAATGGT |
| | | TATATGGGATGGTTCAGGCAGGCCCCAGGAAAGGAGAGAGAAGGGGTTGCCACC |
| | | ATTTATACTGGGGATGGCCGCACCTATTACGCCGATTCCGTGAAGGGGCGGTTC |
| | | ACAATCTCTCGTGACAACGCCAAGAACACTGTCGATCTTCAGATGTCCAGTCTG |
| | | AAACCGGAGGACACTGCCATGTATTACTGTGCCGCACGCGCAGCTCCCCTCTAT |
| | | TCAAGCGGCTCCCCTGTCACTCGCGCACGCTATAACGTGTGGGGGCAGGGAACT |
| | | CAGGTGACTGTGTCTAGC |
| hIL27 | 1297 | CAGGTGCAGCTGCAAGAGAGGGGCGGTGGCAGCGTGCAAGCCGGAGGTTCTCTG |
| Ra_VH | | CGGCTGAGTTGTACCGCTTCTGGATACGTGTCCTGCGACTACTTTTTGCCTTCA |
| H5- | | TGGTATCGGCAGGCCCCAGGCAAAGAACGCGAGTTTGTGTCAGTGATCGACGGC |
| DR594 | | ACTGGTTCCACATCCTACGCAGCCAGCGTGAAGGGCCGTTTTACTGCGTCTCAG |
| | | GATAAAGGCAAGAACATCGCTTATTTGCAAATGAACAGCCTGAAGCCTGAAGAC |
| | | ACAGCCATGTATTACTGCAAGGCAAGCTGCGTTCGCGGTCGGGCGATCTCCGAG |
| | | TACTGGGGGCAGGGTACACAGGTCACCGTCTCCAGTGGAGGTGGCTCCCAGGTG |
| | | CAGCTTCAGGAAAGCGGTGGGGGAAGCGTCCAAGCTGGGGGCTCTCTCCGCCTC |
| | | TCATGTGTGGCCTCTGCCTCCACCTACTGCACTTACGATATGCAGTGGTATCGT |
| | | CAGGCACCAGGGAAGGGACGCGAGTTCGTGTCCGCCATCGACAGCGACGGGACA |
| | | ACCCGGTACGCCGACTCTGTGAAAGGCCGCTTCACCATCAGCCAGGGGACAGCT |
| | | AAAAACACCGTGTACCTGCAAATGAACTCCCTCCAGCCAGAGGATACCGCCATG |
| | | TACTATTGTAAGACCGTATGCGTAGTGGGCAGCAGATGGTCCGACTATTGGGGC |
| | | CAGGGGACGCAGGTCACCGTGTCCAGC |
| hIL27 | 1298 | CAGGTCCAGCTCCAGGAATCCGGTGGCGGTTCCGTGCAGGCAGGGGGGAGCCTG |
| Ra_VH | | CGTCTGAGTTGCACTGCCAGCGGCTACGTTTCATGTGATTACTTCCTGCCCAGT |
| H5- | | TGGTACAGGCAGGCTCCTGGTAAAGAGCGCGAGTTCGTGAGCGTGATTGATGGG |
| DR594 | | ACTGGTTCAACCAGTTACGCGGCCAGCGTGAAGGGGCGCTTCACCGCTTCCCAG |
| | | GACAAGGGTAAGAACATCGCATATCTTCAGATGAACTCTCTCAAGCCTGAAGAC |
| | | ACCGCTATGTACTATTGCAAAGCCAGCTGCGTGCGTGGCCGCGCCATCAGCGAA |
| | | TACTGGGGGCAGGGCACCCAGGTCACTGTGTCTAGCGGTGGCTCAGGAGGGAGC |
| | | GGAGGCTCCGGCCAGGTGCAGGTCCAGGAGTCTGGCGGTGGCAGCGTTCAGGCT |
| | | GGGGGGAGCCTGCGCCTGAGCTGCGTGGCCTCTGCATCCACCTATTGCACCTAC |
| | | GACATGCACTGGTATCGCCAGGCTCCTGGTAAGGGCCGTGAGTTCGTGAGCGCT |
| | | ATTGATAGCGACGGGACTACCCGTTACGCGGATTCTGTGAAGGGCAGATTCACC |
| | | ATCAGCCAGGGCACCGCCAAAAATACTGTTTATCTCCAGATGAATAGCCTGCAA |
| | | CCAGAAGACACCGCCATGTATTACTGCAAGACCGTGTGGGTAGTGGGAAGTAGG |
| | | TGGAGTGATTACTGGGGCCAGGGTACACAAGTCACAGTTTCAAGC |
| hIL27 | 1299 | CAGGTCCAGCTCCAGGAGAGCGGGGGAGGTAGCGTCCAGGGGGGCGGAAGCCTC |
| Ra_VH | | AGACTGAGCTGCACAGCCTCTGGGTACGTCTCCTGCGACTATTTCCTGCCATCA |
| H5- | | TGGTATAGGCAAGCACCAGGCAAGGAGAGGGAGTTCGTGAGTGTGATTGACGGA |
| DR595 | | ACAGGGAGTACCTCCTACGCCGCATCTGTTAAGGGGCGGTTCACTGCAAGTCAG |
| | | GATAAAGGTAAGAACATTGCCTACCTGCAAATGAACTCCCTGAAGCCAGAGGAC |
| | | ACCGCAATGTATTACTGCAAGGCGAGCTGGGTTCGTGGTCGCGCAATCTCCGAG |
| | | TACTGGGGCCAAGGGACCCAGGTGACCGTGTCTTCCGGTGGCGGTTCCCAGGTG |
| | | CAGCTCCAGGAGTCCGGGGGTGGCTCTGTGCAGGCCGGGGGCTCTCTGACCCTC |
| | | TCCTGTGCGGCCAGCGAGTACGCCTACTCTACTTGTAACATGGGGGCGTATCGC |
| | | CAGGCTCCCGGCAAGGAGCGTGAGCTTGTTAGTGCATTTATCTCCGACGGTTCC |
| | | ACCTATTACGCCGACAGCGTGAAGGGCCGCTTTACCATTACCCGTGACAATGCG |
| | | AAGAACACTGTATATTTGCAGATGAACTCCTTGAAGCCCGAAGACACCGCCATC |
| | | TACTATTGCAGCGCTAACTGTTACCGTCGCCTGCGCAACTATTGGGGGCAGGGG |
| | | ACACAGGTGACAGTCTCCTCC |
| hIL27 | 1300 | CAGGTGCAGCTTCAGGAGAGGGGCGGAGGGTCCGTCCAAGCTGGCGGTTCCCTG |
| Ra_VH | | AGACTGAGTTGCACCGCTTCAGGGTATGTTTCCTGTGACTATTTCCTTCCGAGC |
| H5- | | TGGTATCGGCAAGCGCCAGGAAAGGAGCGTGAGTTTGTCAGCGTGATCGACGGC |
| DR595 | | ACCGGAAGCACCTCCTACGCCGCGAGTGTGAAAGGCCGTTTTACTGCATCCCAA |
| | | GACAAAGGCAAGAACATCGCCTACCTTCAAATGAACTCTCTGAAGCCGGAGGAC |
| | | ACCGCCATGTACTATTGCAAGGCATCTTGCGTCAGGGGCAGGGCCATCTCAGAG |
| | | TACTGGGGACAGGGAACCCAGGTGACAGTGTCTTCCGGCGGAAGTGGTGGCTCC |
| | | GGTGGCTCTGGTCAGGTTCAGTTGCAGGAGTCCGGTGGAGGCTCAGTGCAGGCC |
| | | GGTGGCTCCCTGACGCTGTCTTGCGCGGCCAGCGAGTATGCTTATTCCACCTGC |
| | | AATATGGGGTGGTATCGCCAGGCCCCCGGAAAGGAACGTGAGCTGGTGAGCGCC |
| | | TTCATCAGCGACGGGTCTACTTATTACGCTGATTCCGTCAAGGGTAGGTTTACC |
| | | ATCACACGGGATAATGCCAAGAACACCGTCTACCTCCAGATGAACTCTCTGAAG |
| | | CCCGAAGACACTGCCATCTACTATTGCTCCGCAAATTGCTACCGCAGACTGAGA |
| | | AATTATTGGGGCCAGGGAACTCAGGTGACTGTGTCATCA |
| hIL27 | 1301 | CAGGTGCAGGTTCAGGAATCCGGGGGGGGGTCTGTGCAGGCTGGTGGCTCTCTG |
| Ra_VH | | CGCCTCTCTTGCACCGCAAGCGGATACGTGTCGTGTGACTACTTCCTGCCGTCT |
| H5- | | TGGTACAGACAAGCGCCCGGTAAAGAGCGCGAGTTCGTTTCCGTCATTGATGGC |
| DR596 | | ACCGGGAGCACTTCCTACGCAGCCAGCGTAAAGGGCAGATTCACGGCTTCTCAA |
| | | GACAAGGGCAAGAATATCGCGTACCTCCAGATGAACTCTCTGAAGCCCGAGGAC |
| | | ACCGCGATGTACTATTGCAAGGCGAGCTGCGTGAGAGGCAGGGCCATCTCCGAA |
| | | TACTGGGGCCAGGGCACCCAGGTGACCGTGTCTAGCGGTGGGGGTAGCCAGGTA |
| | | CAGTTGCAGGAATCAGGCGGTGGATTGGTTCAGCCGGGTGGAAGCCTGAGGCTT |
| | | AGCTGCACCGCCTCCGGGTTGACTTTCGACGATTCTGTAATGGGCTGGTTTCGC |
| | | CAGGCCCCTGGTAAGGGGAGGGAGGCTGTGAGCTGCATTTCTTCCAGTGGAGCC |
| | | AATGCTTTCTACGCCGACTCCGTCAAGGGGAGATTCACCATCAGCCGCGACAAT |
| | | GCAAAAAACACTCTGTACCTCCAGATGAACTCCTTGAAGCCCGAGGACACTGCC |
| | | ACCTATTACTGTAAAAGAGGACACGCTTGTGCTGGCTACTATCCTATCCCTTAC |
| | | GATGACTATTGGGGTCAGGGCACTCAAGTGACCGTCTCCTCC |
| hIL27 | 1302 | CAGGTGCAGTTGCAGGAGTCTGGAGGCGGAAGCGTCCAAGCCGGGGGTTCCCTG |
| Ra_VH | | AGACTCTCTTGCACCGCCTCCGGCTACGTATCCTGCGACTACTTCCTGCCCAGC |
| H5- | | TGGTATCGCCAGGCTCCCGGTAAGGAGCGCGAGTTTGTCAGCGTGATCGACGGC |
| DR596 | | ACCGGCTCCACCTCTTATGCCGCTAGTGTTAAGGGCCGGTTTACTGCCTGCCAG |
| | | GACAAGGGCAAAAACATTGCCTACCTCCAGATGAACAGCCTGAAGCCCGAGGAC |
| | | ACCGCAATGTATTACTGCAAGGCGTCCTGCGTGAGGGGTCGTGCCATTTCTGAG |
| | | TACTGGGGCCAGGGGACCCAAGTCACCGTAAGTTCTGGGGGCTCTGGGGGTAGC |
| | | GGAGGGAGTGGACAGGTGCAGCTCCAGGAGAGCGGCGGAGGCCTGGTGCAGCCT |
| | | GGTGGGTCACTGAGACTGTCCTGCACAGCTTCAGGCCTGACCTTTGACGATTCC |
| | | GTCATGGGGTGGTTCAGGCAAGCTCCGGGGAAAGGGCGCGAGGCCGTAAGCTGC |
| | | ATCAGTAGCTCTGGTGCCAATGCTTTCTACGCGGACTCGGTGAAGGGGCGCTTT |
| | | ACAATCTCTCGCGATAATGCTAAGAACACCTTGTATCTCCAAATGAACTCTTTG |
| | | AAGCCCGAGGACACCGCCACCTACTATTGTAAGAGAGGGCACGCATGTGCTGGG |
| | | TATTACCCTATCCCTTATGATGACTACTGGGGGCAAGGAACACAGGTTACTGTG |
| | | TCTTCC |
| hIL27 | 1303 | CAGGTGCAGGTCCAGGAGTCTGGAGGTGGCCTGGTGCAGCCCGGGGGAAGCCTT |
| Ra_VH | | CGCCTGAGTTGTGCGGCATCCGGCTTTTCCTTCTCATCTTACGCGATGAAGTGG |
| H6- | | GTCCGCCAGGCCCCAGGGAAAGGCCTGGAGTGGGTGAGTACCATTTCATCCGGC |
| DR591 | | GGTAGCTCCACCAATTATGCTGACAGCGTGAAGGGTAGGTTCACCATCAGCCGC |
| | | GATAATGCCAAGAACACCCTCTACCTCCAGTTGAACTCCCTGAAGATCGAAGAC |
| | | ACAGCTATGTATTACTGCGCCAAGGCCATCGTTCCCACTGGGGCCACAATGGAG |
| | | AGGGGACAGGGGACGCAGGTGACTGTTAGCTCCGGTGGGGGCAGCCAAGTACAG |
| | | TTGCAGGAGTCTGGTGGCGGTTCCGTGCAGGCCGGGGGCTCACTGCGTCTGTCC |
| | | TGTACCGGTTCTGGTGCTATCGCCTCTGGATACATTGATTCTCGCTGGTGCATG |
| | | GCGTGGTTCAGGCAAGCCCCTGGTAAAGAAAGAGAGGGTGTGGCTGCCATCTGG |
| | | CCAGGAGGGGGGCTCACTGTATACGCAGATTCTGTGAAGGGAAGATTCACTATC |
| | | TCACGGGATCACGCCAAAAATACGCTGTATTTGCAGATGAACAATTTGAAGCCG |
| | | GAGGACACCGCGATGTACTATTGTGCTGCGGGCTCTCCTCGTATGTGCCCCTCC |
| | | CTGGAGTTCGGATTCGATTATTGGGGCCAGGGCACGCAAGTTACCGTAAGCAGT |
| hIL27 | 1304 | CAGGTCCAGTTGCAGGAGAGTGGTGGGGGTCTCGTGCAACCGGGAGGCAGCCTC |
| Ra_VH | | AGGCTGTCCTGCGCTGCCAGCGGGTTTTCCTTCAGCTCTTATGCGATGAAATGG |
| H6- | | GTCCGCCAGGCTCCAGGCAAAGGTCTGGAGTGGGTGAGCACCATCAGCTCAGGA |
| DR591 | | GGCTCCAGCACGAACTACGCTGACTCCGTTAAGGGACGGTTCACTATCTCAAGA |
| | | GACAATGCAAAAAATACTCTGTATCTCCAGCTGAACTCCCTGAAGATCGAAGAC |
| | | ACTGCTATGTATTACTGTGCCAAGGCCATCGTTCCTACAGGCGCGACAATGGAG |
| | | CGTGGCCAGGGCACCCAGGTCACAGTGTCTTCAGGAGGCAGCGGTGGAAGCGGG |
| | | GGCTCTGGCCAAGTGCAACTCCAGGAGTCAGGGGGGGGGTCCGTGCAGGCAGGA |
| | | GGCAGCCTTCGCCTGTCCTGTACCGCATCAGGCGCTATTGCAAGCGGCTACATC |
| | | GACTCCCGGTGGTGCATGGCCTGGTTCCGCCAAGCGCCGGGCAAAGAGCGCGAG |
| | | GGCGTGGCTGCAATCTGGCCTGGTGGAGGCTTGACAGTGTATGCCGATAGTGTG |
| | | AAGGGCCGCTTCACAATCAGCCGGGATCACGCAAAGAATACCCTGTACCTCCAG |
| | | ATGAATAACCTGAAGCCTGAAGACACCGCCATGTACTATTGCGCAGCTGGCAGT |
| | | CCTCGCATGTGTCCCAGCCTGGAGTTTGGATTCGATTACTGGGGTCAAGGTACA |
| | | CAGGTGACTGTCTCATCC |
| hIL27 | 1305 | CAAGTGCAGCTCCAGGAGTCAGGGGGGGGCCTTGTGCAGCCTGGTGGCTCTCTG |
| Ra_VH | | AGGCTGTCTTGTGCGGCTTCAGGCTTCTCCTTCTCCTCATACGCAATGAAGTGG |
| H6- | | GTACGTCAGGCCCCCGGCAAAGGCCTTGAGTGGGTCTCCACTATGTCTTCCGGT |
| DR592 | | GGCTCTTCCACCAACTACGCTGATTCAGTCAAAGGCCGGTTCACGATCTCTCGC |
| | | GATAACGCCAAGAACACGCTGTACCTCCAGCTGAACTCCCTGAAGATCGAAGAC |
| | | ACTGCCATGTACTATTGCGCCAAGGCCATTGTTCCCACCGGAGCCACTATGGAA |
| | | CGCGGCCAGGGCACCCAGGTCACCGTGTCCTCTGGAGGGGGTTCACAGGTCCAA |
| | | CTTCAGGAGAGCGGAGGGGGGTCCGTGCAGGGGGGAGGCTCCCTGCGTCTGTCC |
| | | TGTACCGCTCCCGGTTTCACCAGTAACTCCTGGGGCATGGATTGGTACAGACAG |
| | | GCCCCTGGTAAGGAAAGGGAATTTGTCAGCTCTATCAGCACCGACGGAACAACC |
| | | GGCTATGCCGATAGCGTGAAGGGACGCTTTACTATCTCAAAGGACAAGGCTAAG |
| | | GACACAGTGTACCTCCAGATGAACTCCGTCAAGCCCGAGGACACCGGCATGTAT |
| | | TCCTGCAAGACAAAAGACGGCACCATTGCAACTATGGAGCTGTGTGATTTTGGT |
| | | TATTGGGGCCAGGGAACACAGGTCACCGTCTCCAGC |
| hIL27 | 1306 | CAGGTTCAGCTTCAGGAGTCTGGCGGAGGCCTGGTTCAGCCCGGAGGCAGTCTG |
| Ra_VH | | CGCCTTTCTTGCGCCGCGAGCGGCTTCTCATTGCCCAGCTACGCCATGAAGTGG |
| H6- | | GTGCGCCAGGCTCCCGGAAAGGGACTGGAGTGGGTGTCCACAATCTCTTCAGGA |
| DR592 | | GGCTCCAGCACAAATTACGCCGACAGCGTGAAGGGCCGCTTCACGATCTCACGG |
| | | GATAACGCGAAGAACACTCTGTATCTTCAGCTCAACTCCCTGAAGATCGAGGAC |
| | | ACAGCTATGTATTACTGTGCTAAGGCTATCGTTCCTACCGGCGCTACTATGGAA |
| | | AGAGGGCAGGGCACCCAAGTGACTGTGTCTAGCGGAGGTAGCGGAGGGTCCGGT |
| | | GGAAGTGGTCAGGTACAGCTCCAGGAATCTGGTGGAGGCAGTGTTCAGGCTGGA |
| | | GGTTCTTTGAGACTGTCCTGCACAGCTCCTGGATTTACCAGCAACAGCTCGGGG |
| | | ATGGACTGGTATCGCCAGGCACCGGGCAAGGAAAGAGAGTTTGTGTCTAGCATC |
| | | TCTACCGACGGTACAACCGGCTATGCCGACTCTGTGAAGGGTAGGTTCACTATC |
| | | TCCAAGGACAAAGCAAAGGATACTGTGTACCTCCAGATGAACTCGTTGAAGCCC |
| | | GAGGACACCGGCATGTACTCATGTAAGACCAAGGATGGGACCATCGCTACTATG |
| | | GAGCTGTGTGACTTCGGATACTGGGGCCAGGGTACTCAGGTCACCGTGTCTTCC |
| hIL27 | 1307 | CAAGTCCAGTTGCAGGAGTCCGGCGGGGGCCTGGTCCAGCCAGGAGGCAGCTTG |
| Ra_VH | | CGTCTGTCATGTGCTGCCTCTGGTTTCTCATTTCCAGCTACGCCATGAAGTTGG |
| H6- | | GTGAGACAGGCTCCAGGAAAGGGCCTGGAGTGGGTGTCTACAATGTCCTCTGGA |
| DR593 | | GGGTCTTCCACTAACTATGCCGACTCCGTCAAAGGACGCTTCACAATTTCACGC |
| | | GACAACGCGAAAAATACCTTGTACCTCCAGTTGAACTCACTCAAGATCGAAGAC |
| | | ACGGCCATGTATTACTGCGCCAAAGCCATCGTGCCCACAGGTGCTACTATGGAG |
| | | CGCGGCCAGGGCACCCAGGTTACCGTCAGCTCTGGCGGAGGCTCCCAAGTGCAG |
| | | CTGCAAGAGAGCGGCGGGGGCAGCGTCCAGGCCGGTGGCTCCCTGAGGCTCTCC |
| | | TGTGCTGCCTCCGGTTACCCATACTCCAATGGGTATATGGGCTGGTTCCGCCAA |
| | | GCCCCAGGCAAGGAGAGGGAAGGAGTGGCAACCATCTACACCGGCGATGGCCGC |
| | | ACATATTACGCAGACAGCGTCAAGGGCAGGTTTACCATTAGTCGCGACAACGCC |
| | | AAGAACACTGTGGACCTCCAGATGTCTTCCCTGAAGCCCGAGGACACAGCTATG |
| | | TATTACTGTGCGGCCAGGGCTGCACCGCTGTACTCCAGCGGTTCACCGCTGACA |
| | | CGCGCCCGGTACAACGTCTGGGGCCAGGGCACACAGGTTACGGTCTCTTCT |
| hIL27 | 1308 | CAGGTCCAGCTCCAGGAATCCGGTGGGGGCCTGGTGCAGCCCGGTGGCTCCCTG |
| Ra_VH | | CGCCTGTCCTGCGCTGCATCCGGCTTCAGCTTCAGCTCCTATGCTATGAAGTGG |
| H6- | | GTCCGGCAAGCGCCTGGCAAGGGACTGGAGTGGGTGAGTACAATCAGCCCCGGG |
| DR593 | | GGCTCCTCTACCAACTATGCCGATAGCGTGAAGGGTAGGTTCACCATTAGCCGC |
| | | GACAACGCCAAGAACACTCTGTACCTTCAGCTGAACTCCCTGAAAATCGAAGAC |
| | | ACAGCGATGTATTACTGCGCGAAGGCCATCGTGCCTACTGGTGCTACTATGGAA |
| | | AGAGGGCAAGGCACACAGGTGACCGTTAGCTCCGGTGGCTCCGGCGGAAGCGGT |
| | | GGGTCAGGTCAGGTTCAGCTCCAAGAAAGCGGCGGTGGCAGCGTGCAGGCTGGA |
| | | GGCTCCCTTAGACTCTCCTGCGCCGCATCTGGATACCCTTATAGCAACGGCTAC |
| | | ATGGGATGGTTTCGCCAAGCCCCTGGTAAGGAGCGCGAGGGCGTCGCTACTATT |
| | | TATACTGGGGATGGCCGCACTTACTATGCCGATTCTGTGAAGGGGCGCTTTACT |
| | | ATCTCACGCGACAACGCAAAGAACACCGTGGACCTCCAGATGTCATCTCTCAAG |
| | | CCGGAAGATACCGCTATGTATTACTGTGCTGCCCGCGCAGCTCCCCTGTACTCC |
| | | TCCGGCTCCCCACTCACCCGTGCCCGCTATAACGTGTGGGGACAGGGCACTCAG |
| | | GTGACCGTCTCATCT |
| hIL27 | 1309 | CAGGTGCAGTTGCAGGAGAGCGGGGGGGGCTGGTCCAGCCCGGCGGGATCTCTG |
| Ra_VH | | CGCCTCAGCTGCGCCGCAAGCGGCTTCTCCTTCTCCAGCTACGCTATGAAATGG |
| H6- | | GTCCGCCAGGCCCCCGGTAAGGGGTTGGAGTGGGTGTCTACCATTTCCAGTGGC |
| DR594 | | GGTTCCAGCACCAACTACGCTGACAGCGTGAAGGGCAGATTCACGATCTCTCGC |
| | | GACAATGCTAAAAATACCCTGTACCTGCAACTCAACAGCCTGAAGATCGAGGAT |
| | | ACCGCCATGTATTACTGTGCCAAGGCCATCGTTCCTACTGGGGCCACTATGGAG |
| | | AGGGGACAAGGAACTCAGGTCACCGTCTCTTCCGGTGGGGGCAGCCAAGTCCAG |
| | | CTCCAAGAATCTGGGGGAGGCTCTGTGCAGGCTGGGGGATCACTCAGACTGTCC |
| | | TGTGTGGCGTCCGCTTCCACCTATTGCACCTACGACATGCACTGGTATCGCCAG |
| | | GCCCCTGGCAAAGGCAGGGAATTTGTGTCTGCTATCGACAGCGACGGTACAACC |
| | | CGCTATGCCGATTCAGTGAAGGGTCGGTTCACCATCTCACAAGGCACCGCCAAG |
| | | AACACCGTGTACTTGCAGATGAACTCTCTGCAACCCGAGGACACTGCAATGTAT |
| | | TACTGCAAGACTGTGTGCGTGGTCGGAAGCAGATGGTCTGATTATTGGGGCCAA |
| | | GGCACACAGGTTACTGTGAGCAGT |
| hIL27 | 1310 | CAGGTGCAGCTCCAGGAGTCCGGGGGAGGCCTGGTGCAGCCTGGTGGGTCTCTG |
| Ra_VH | | CGGCTGTCCTGTGCCGCTTCAGGATTTAGCTTCTCTTCCTATGCTATGAAGTGG |
| H6- | | GTGCGCCAGGCTCCTGGTAAGGGACTGGAATGGGTGTCTACAATTAGTTCCGGC |
| DR594 | | GGGTCCTCTACCAACTATGCCGACTCCGTGAAGGGTAGATTCACGATCAGCAGA |
| | | GACAACGCCAAGAATACCCTCTACTTGCAGCTCAACTCCCTGAAAATTGAAGAC |
| | | ACCGCGATGTATTACTGTGCCAAGGCCATCGTGCCCACGGGCGCTACAATGGAG |
| | | CGCGGCCAGGGCACACAGGTGACCGTTTCTTCCGGTGGCAGCGGAGGCTCCGGG |
| | | GGCTCCGGCCAGGTGCAGCTCCAGGAGTCAGGAGGGGGGTCCGTGCAGGCCGGT |
| | | GGCAGTCTGCGTTTGTCCTGTGTGGCCAGCGCATCCACCTATTGTACCTACGAT |
| | | ATGCACTGGTATCGTCAGGCCCCAGGCAAGGGCAGGGAGTTCGTTAGCGCTATT |
| | | GACTCCGACGGCACCACTCGCTACGCTGACAGTGTCAAAGGCCGGTTCACTATC |
| | | TCTCAGGGGACTGCCAAGAACACCGTGTATCTCCAGATGAACTCCCTGCAACCA |
| | | GAGGACACAGCCATGTACTATTGCAAGACAGTGTGCGTTGTGGGGTCTCGCTGG |
| | | TCCGACTATTGGGGCCAGGGCACCCAGGTGACAGTTGTCTTCC |
| hIL27 | 1311 | CAAGTGCAGCTCCAGGAGTCTGGTGGAGGCCTGGTCCAGCCCGGTGGGAGCTTG |
| Ra_VH | | AGATTGTCTTGCGCAGCCTCTGGATTCTCTTTCTCCAGCTACGCCATGAAATGG |
| H6- | | GTGCGGCAGGCACCAGGGAAGGGCCTGGAATGGGTGTCTACCATTTCTTCCGGC |
| DR595 | | GGTTCTTCCACTAATTACGCAGACTCCGTGAAGGGCCGGTTCACCATTTCCCGT |
| | | GACAACGCTAAGAATACCCTGTACTTGCAGCTGAACTCCCTGAAGATCGAGGAG |
| | | ACGGCTATGTATTACTGTGCTAAAGCCATTGTCCCTACAGGGGCCACAATGGAA |
| | | CGCGGCCAGGGTACTCAAGTGACGGTCAGCTCTGGGGGGGGCAGCCAAGTGCAG |
| | | CTCCAGGAGAGCGGAGGTGGGTCCGTGCAGGCTGGTGGCTCCCTGACCCTGAGC |
| | | TGTGCCGCATCTGAATATGCGTATAGCACTTGTAATATGGGATGGTATCGCCAG |
| | | GCTCCTGGTAAGGAGCGCGAGCTGGTGAGCGCTTTCATTTCCGACGGTAGCACA |
| | | TATTACGCGGACTCCGTCAAAGGTCGGTTCACCATTACACGCGATAACGCCAAG |
| | | AATACCGTGTATTTGCAGATGAACTCACTCAAGCCAGAAGACACTGCCATCTAC |
| | | TATTGTTCAGCCAACTGTTATCGCAGACTCCGCAACTATTGGGGCCAGGGGACG |
| | | CAGGTAACCGTGTCCTCC |
| hIL27 | 1312 | CAGGTGCAGCTCCAGGAATCTGGCGGTGGCCTGGTGCAGCCGGGGGGGTCCCTG |
| Ra_VH | | AGACTGAGTTGCGCTGCCAGCGGGTTTTCCTTCAGTTCGTATGCCATGAAGTGG |
| H6- | | GTCCGCCAAGCTCCGGGGAAGGGCCTGGAATGGGTCTCCACTATTAGTTCCGGT |
| DR595 | | GGCTCTAGCACAAACTATGCGGATTCCGTGAAGGGACGCTTCACCATCTCTCGG |
| | | GACAACGCCAAGAATACCCTGTACCTCCAGCTGAATAGTCTCAAGATCGAAGAC |
| | | ACCGCGATGTACTATTGCGCCAAAGCCATCGTTCCTACTGGTGCTACAATGGAG |
| | | CGTGGCCAGGGCACCCAGGTGACCGTCTCCTCTGGCGGATCTGGGGGTTCCGGG |
| | | GGCTCCGGCCAGGTCCAGTTGCAGGAAAGCGGGGGGGGCTCCGTTCAGGCCGGG |
| | | GGCAGTCTGACCCTGTCCTGTGCAGCCTCTGAGTACGCCTATTCCACCTGTAAC |
| | | ATGGGCTGGTACAGACAAGCGCCCGGAAAAGAGCGCGAATTGGTCTCCGCCTTC |
| | | ATCTCCGATGGTAGCACTTATTACGCGGACTCTGTAAAAGGCCGTTTCACTATT |
| | | ACCCGCGACAACGCCAAGAACACCGTCTATCTCCAGATGAACAGCCTGAAGCCA |
| | | GAAGATACGGCCATTTACTATTGCTCCGCTAACTGCTACAGACGGTTGAGGAAT |
| | | TATTGGGGCCAGGGCACACAAGTGACTGTTTCCTCC |
| hIL27 | 1313 | CAGGTGCAGCTGCAAGAGAGCGGTGGCGGTCTGGTACAGCCAGGTGGCAGCCTG |
| Ra_VH | | CGCCTGTCCTGCGCTGCATCAGGCTTCTCCTTCAGTTCCTATGCCATGAAGTGG |
| H6- | | GTGCGCCAGGCTCCAGGCAAAGGATTGGAGTGGGTGTCTACCATTTCTTCCGGG |
| DR596 | | GGCTCATCCACAAACTACGCCGACAGCGTGAAGGGCCGCTTCACCATCTCCCGC |
| | | GACAACGCCAAGAACACTCTGTATCTTCAGCTGAACTCCCTGAAGATTGAGGAT |
| | | ACCGCTATGTATTACTGCGCTAAGGCCATCGTTCCCACAGGCGCTACGATGGAA |
| | | CGCGGTCAGGGGACACAAGTAACCGTAAGCAGTGGTGGGGGTAGCCAGGTGCAG |
| | | CTCCAGGAGTCCGGCGGTGGCCTGGTGCAGCCGGGTGGGTCCCTGAGGCTGAGT |
| | | TGCACAGCCAGCGGTCTGACCTTCGATGACTCCGTGATGGGCGGGTTCCGCCAG |
| | | GCACCTGGCAAGGGCCGCGAGGCTGTGTCCTGCATTAGCTCTAGCGGTGCCAAT |
| | | GCCTTCTACGCTGACTCAGTCAAGGGCCGCTTCACCATCTCCCGCGATAACGCC |
| | | AAGAACACCCTGTACTTGCAGATGAACTCCCTGAAACCCGAGGATACAGCAACA |
| | | TACTATTGCAAGCGGGGGCACGCCTGTGCAGGATACTATCCTATCCCCTATGAC |
| | | GATTATTGGGGTCAGGGCACCCAGGTCACCGTCAGCAGC |
| hIL27 | 1314 | CAGGTGCAGCTCCAGGAGTCCGGCGGAGGTCTGGTTCAGCCCGGAGGCTCCCTT |
| Ra_VH | | CGCCTCTCCTGTGCTGCGTCAGGATTCTCCTTTAGCTCCTACGCTATGAAGTGG |
| H6- | | GTGCGCCAAGCCCCCGGCAAGGGCCTGGAGTGGGTATCCACCATTTCCAGTGGC |
| DR596 | | GGTTCTTCAACCAACTATGCTGATTCCGTGAAAGGGCGCTTCACCATCTCCCGT |
| | | GACAACGCAAAAAATACCTTGTATTTGCAGCTGAACAGCCTGAAGATCGAGGAC |
| | | ACCGCGATGTACTATTGCGCCAAGGCCATCGTGCCTACTGGCGCAACAATGGAG |
| | | CGTGGACAGGGCACTCAGGTCACTGTGAGTAGCGGAGGGAGTGGCGGTTCCGGC |
| | | GGTTCAGGACAGGTGCAGCTCCAGGAGAGCGGGGGTGGGCTGGTACAGCCAGGT |
| | | GGCTCTCTCAGGCTGAGTTGCACGGCCTCAGGGCTGACCTTCGATGACAGCGTC |
| | | ATGGGCTGGTTCCGCCAGGCCCCAGGTAAAGGCCGTGAGGCTGTGTCCCGTATT |
| | | AGCTCATCTGGGGCCAATGCGTTCTATGCTGACAGTGTCAAAGGCCGCTTCACA |
| | | ATTTCCCGTGACAACGCCAAGAACACCGTGTATTTGCAGATGAACAGTCTGAAG |
| | | CCGGAAGACACCGCCACCTACTATTGTAAGCGCGGTCACGCTTGTGCAGGTTAT |
| | | TACCCTATTCCTTATGATGACTACTGGGGCCAGGGAACGCAGGTGACAGTGTCC |
| | | TCC |
| hIL27 | 1315 | CAGGTCCAGTTGCAGGAGTCTGGAGGTGGCCTGGTGCAGCCGGGAGGTTCTCTG |
| Ra_VH | | CGGCTGTCCTGCGCCGCATCTGGATTCACCTTCAGTTCTTATCCAATGTCTTGG |
| H7- | | GTCAGGCAAGCGCCGGGCAAGGGCCTGGAATGGATTAGCACTATTTCCGCAGGT |
| DR591 | | GGCGATACCACTCTGTATGCCGATTCAGTGAAAGGCCGGTTCACCAGCTCTCGG |
| | | GATAACGCTAAGAACACCCTGTATCTCCAGCTGAATAGCCTTAAAACCGAAGAC |
| | | ACCGCAATCTATTACTGTGCTAAAAGGATTGATTGTAATTCTGGCTATTGTTAC |
| | | AGACGCAACTACTGGGGACAGGGCACCCAAGTGACCGTTTCCAGTGGGGGGGGG |
| | | AGCCAGGTACAACTCCAGGAGTCCGGTGGAGGTAGCGTGCAGGCCGGGGGCTCT |
| | | CTCCGTCTGTCCTGCACCGCATCCGGGGCTATCGCCTCTGGGTACATTGACAGC |
| | | CGCTGGTGCATGGCCTGGTTCCGTCAGGCCCCCGGCAAGGAACGCGAGGGCGTG |
| | | GCTGCCATCTGGCCGGGCGGAGGGCTGACTGTGTATGCGGACTCCGTCAAGGGC |
| | | AGGTTTACCATCAGCCGCGACCATGCTAAGAACACCCTGTACCTCCAGATGAAT |
| | | AACTTGAAACCTGAAGACACCGCCATGTACTATTGCGCGGCTGGTTCCCCACGC |
| | | ATGTGCCCCTCACTTGAGTTTGGGTTCGATTACTGGGGCCAGGGCACCCAGGTG |
| | | ACTGTGTCCAGC |
| hIL27 | 1316 | CAGGTCCAGCTTCAGGAGTCCGGCGGTGGCCTTGTCCAGCCAGGAGGCAGCCTG |
| Ra_VH | | CGGCTGTCCTGCGCCGCGAGCGGCTTCACGTTCTCATCCTACCCAATGTCCTGG |
| H7- | | GTCCGTCAGGCCCCAGGGAAGGGGCTGGAGTGGATCAGTACCATCTCCGCCGGT |
| DR591 | | GGCGATACCACTTTGTACGCCGACTCTGTGAAAGGGCGCTTTACCTCTTCCCGC |
| | | GATAACGCGAAAAACACATTGTACCTCCAGCTCAACTCCCTGAAGACTGAGGAC |
| | | ACTGCCATCTACTATTGCGCCAAGAGAATTGATTGCAACAGGGGGTATTGCTAC |
| | | CGGCGCAACTACTGGGGCCAGGGAACCCAGGTGACGGTATCCTCTGGGGGCTCT |
| | | GGAGGTTCAGGAGGCAGCGGACAAGTGCAGCTCCAGGAATCAGGAGGCGGTTCT |
| | | GTGCAGGCGGGGGCTCTCTGCGTCTGAGCTGCACTGCGTCTGGACGCCATCGCT |
| | | TCTGGTTACATCGACAGCAGATGGTGCATGGCATGGTTCCGTCAGGCTCCGGGA |
| | | AAGGAGAGGGAGGGAGTGGCCGCTATCTGGCCCGGAGGGGGCTTGACAGTCTAC |
| | | GCTGATTCCGTGAAGGGAAGGTTCACGATCTCACGTGACCACGCTAAGAATACC |
| | | CTGTACCTTCAGATGAATAACCTGAAGCCCGAGGACACCGCAATGTACTATTGT |
| | | GCTGCCGGAAGCCCCAGGATGTGTCCCAGCCTGGAATTTGGCTTTGACTACTGG |
| | | GGTCAGGGCACTCAGGTCACAGTCTCCTCT |
| hIL27 | 1317 | CAGGTGCAGTTGCAGGAGTCCGGGGGGGGCCTGGTGCAGCCAGGAGGGTCTCTG |
| Ra_VH | | AGGCTGAGTTGCGCCGCTAGTGGCTTCACCTTCTCCAGCTATCCCATGTCTTGG |
| H7- | | GTGCGCCAGGCTCCCGGTAAAGGACTTGAGTGGATTAGCACGATCTCCGCTGGA |
| DR592 | | GGTGACACGACTCTTCTACGCCGATAGCGTGAAGGGACGCTCACCAGTTCTCGT |
| | | GACAACGCCAAGAATACCCTGTACCTCCAACTGAACAGCCTGAAGACGGAGGAC |
| | | ACCGCCATCTATTACTGTGCAAAGCGTATTGATTGTAACTCAGGATACTGCTAT |
| | | CGGCGCAACTATTGGGGACAGGGCACGCAGGTGACAGTTAGCTCCGGTGGGGGA |
| | | AGTCAGGTCCAACTTCAGGAAAGCGGAGGTGGCAGCGTTCAGGCTGGCGGTTCC |
| | | TTGCGCCTGTCTTGTACCGCACCGGGCTTTACAAGCAACAGCTGGGGTATGGAC |
| | | TGGTATAGACAAGCTCCAGGCAAAGAAAGGGAGTTCGTGTCCAGCATTAGCACT |
| | | GACGGCACCACAGGCTATGCTGATTCCGTGAAGGGACGCTTCACTATCTCCAAA |
| | | GATAAGGCGAAGGACACTGTATACCTCCAGATGAACTCCCTGAAGCCTGAGGAT |
| | | ACCGGCATGTATAGTTGCAAGACCAAGGATGGCACCATCGCAACGATGGAACTC |
| | | TGTGACTTCGGCTATTGGGGGCAGGGCACCCAGGTGACCGTGTCCTCT |
| hIL27 | 1318 | CAGGTGCAGCTGCAAGAATCCGGGGGCGGGCTCGTGCAGCCTGGGGGTTCCCTC |
| Ra_VH | | CGTCTGAGCTGCGCGGCTTCCGGCTTCACTTTCTCTTCCTATCCCATGTCCTGG |
| H7- | | GTGAGGCAGGCCCCTGGCAAGGGCTTGGAATGGATTAGCACCATCAGCGCCGGG |
| DR592 | | GGCGACACAACCCTGTACGCTGACAGCGTGAAGGGAAGATTCACGTCCAGCCGG |
| | | GATAACGCCAAGAACACCCTGTACCTCCAGTTGAACTCCCTGAAGACTGAAGAT |
| | | ACCGCCATCTATTACTGTGCCAAGCGCATTGATTGCAACAGCGGGTACTGCTAT |
| | | CGCCGGAACTACTGGGGGCAGGGTACACAGGTAACGGTGTCCTCAGGGGGCTCC |
| | | GGGGGGAGTGGGGGCAGCGGGCAAGTTCAGTTGCAGGAGAGCGGGGGGGGCTCC |
| | | GTCCAAGCAGGCGGAAGCCTTCGCCTCAGCTGCACCGCACCGGGTTTCACATCT |
| | | AATAGCTGCGGAATGGATTGGTATCGGCAGGCCCCCGGTAAGGAGCGCGAGTTC |
| | | GTGAGTTCCATCAGTACCGACGGCACAACCGGCTATGCTGACTCCGTGAAGGGT |
| | | CGCTTCACCATTTCTAAGGATAAGGCCAAAGACACCGTGTACCTTCAGATGAAC |
| | | TCACTGAAGCCAGAAGACACAGGAATGTACTCTTGTAAAACGAAGGATGGAACC |
| | | ATCGCTACGATGGAGCTGTGCGACTTTGGCTACTGGGGCCAGGGCACCCAGGTG |
| | | ACAGTAAGCTCT |
| hIL27 | 1319 | CAGGTCCAACTCCAGGAAAGCGGCGGTGGCCTGGTGCAGCCAGGTGGGTCTCTC |
| Ra_VH | | CGTCTGAGCTGTGCTGCATCCGGCTTCACTTTTAGCTCTTACCCGATGAGCTGG |
| H7- | | GTGCGCCAGGCTCCGGGAAAGGGCCTTGAGTGGATCAGCACCATCTCAGCCGGG |
| DR593 | | GGCGATACAACCCTGTATGCAGACAGTGTTAAAGGCCGTTTTACCTCCTCTCGC |
| | | GACAATGCTAAAAACACCCTTTATCCTCAGCTCAATAGTCTGAAGACTGAGGAC |
| | | ACCGCCATTTATTACTGCGCTAAACGCATTGATTGCAACTCCGGGTACTGCTAC |
| | | AGACGTAACTATTGGGGCCAGGGAACACAGGTGACCGTGTCCAGTGGCGGAGGT |
| | | AGTCAGGTCCAGTTGCAAGAGAGCGGGGGAGGCAGCGTCCAGGGGCGGGGCTCC |
| | | CTGCGCCTGAGCTGCGCAGCCTCTGGTTACCCATACAGCAACGGCTACATGGGG |
| | | TGGTTCAGGCAAGCACCGGGTAAGGAGCGGGAAGGCGTGGCAACCATCTATACA |
| | | GGTGATGGTCGCACCTATTACGCAGATTCCGTCAAGGGCCGGTTCACTATCAGC |
| | | CGCGACAACGCAAAGAATACAGTCGATCTCCAGATGAGCAGTTTGAAACCTGAA |
| | | GACACGGCGATGTATTACTGCGCCGCTCGTGCTGCGCCACTCTACTCCTCTGGC |
| | | TCCCCTCTCACACGTGCTCGTTATAATGTATGGGGCCAGGGCACTCAAGTGACA |
| | | GTCTCCAGT |
| hIL27 | 1320 | CAGGTGCAGTTGCAGGAGTCCGGCGGTGGACTGGTCCACACCAGGGGTAGTCTC |
| Ra_VH | | AGGCTGTCCTGTGCCGCTTCTGGTTTCACTTTCAGCTCCTACCCAATGTCCTGG |
| H7- | | GTACGGCAAGCTCCTGGGAAGGGTCTGGAGTGGATCTCCACGATCTCCGCAGGC |
| DR593 | | GGGGACACTACCCTCTACGCCGACTCTGTGAAGGGCCGTTTCACTTCTAGCAGA |
| | | GATAATGCAAAAAACACTCTCTATCTTCAGCTGAACAGTCTGAAAACCGAAGAT |
| | | ACTGCGATCTATTACTGCGCCAAGCGCATCGACTGCAATTCTGGTTACTGTTAC |
| | | CGTCGCAATTACTGGGGCCAAGGAACCCAGGTCACGGTGTCCTCAGGTGGCAGG |
| | | GGAGGTTCCGGCGGGTCCGGTCAGGTCCAGTTGCAGGAAAGCGGGGGAGGTTCT |
| | | GTCCAAGCCGGAGGCAGCCTGAGGCTGTCTTGCGCTGCCAGTGGATACCCCTAT |
| | | AGCAACGGCTACATGGGCTGGTTCCGCCAAGCGCCAGGGAAGGAACGTGAAGGT |
| | | GTGGCCACCATCTACACCGGCGATGGCAGGACCTACTATGCTGACTCCGTGAAG |
| | | GGTAGGTTCACCATCTCTCGCGATAACGCCAAGAACACTGTGGACCTCCAGATG |
| | | TCTTCCTTGAAACCTGAGGACACCGCGATGTATTACTGCGCGGCCCGCGCCGGG |
| | | CCCCTCTACAGTTCAGGCAGCCCCCTGACCAGAGCAAGGTATAACGTGTGGGGT |
| | | CAGGGCACTCAGGTGACCGTTAGCAGT |
| hIL27 | 1321 | CAAGTTCAGGTCCAGGAATCTGGGGGTGGCCTGGTCCAGCCAGGTGGCTCCCTG |
| Ra_VH | | CGCCTGAGTTGTGCGGCTAGTGGCTTCACCTTCAGCTCCTACCCGATGAGTTGG |
| H7- | | GTGCGTCAGGCTCCAGGGAAGGGTCTGGAGTGGATCTCTACCATCAGCGCCGGC |
| DR594 | | GGCGATACTACCCTGTATGCGGATTCCGTGAAGGGTCGCTTTACCAGCTCTCGG |
| | | GACAACGCCAAGAACACGCTCTACTTGCAGCTGAACAGTGTGAAGACCGAGGAT |
| | | ACCGCTATCTACTATTGTGCCAAGCGCATCGACTGTAACAGGGGGTACTGCTAC |
| | | CGCCGTAACTATTGGGGCCAGGGAACACAAGTTACCGTGTCAAGCGGAGGCGGT |
| | | TCCCAGGTGCAGCTCCAGGAATCTGGCGGAGGCAGCGTACAGGGGGGAGGCTCA |
| | | CTGCGCCTGTCTTGTGTCGCATCAGCCAGCACCTACTGCACCTATGACATGCAC |
| | | TGGTATCGGCAGGCTCCCGGCAAAGGCCGCGAGTTCGTCAGCGCCATTGATTCC |
| | | GATGGCACCACGAGGTACGCCGATTCTGTAAAGGGCCGGTTTACTATCTCCCAG |
| | | GGTACTGCCAAGAACACCGTGTACCTCCAGATGAACAGTCTTCAGCCAGAGGAT |
| | | ACGGCTATGTATTACTGCAAGACTGTTTGTGTCGTTGGCTCCAGGTGGAGCGAC |
| | | TACTGGGGTCAGGGAACCCAGGTCACTGTGAGTAGC |
| hIL27 | 1322 | CAGGTCCAACTTCAGGAATCCGGCGGGGGTCTGGTGCAACCTGGGGGTAGCCTC |
| Ra_VH | | CGGCTTAGTTGCGCCGCAAGTGGATTCACCTTTTCCAGCTATCCCATGTCCTGG |
| H7- | | GTGCGTCAAGCGCCGGGTAAGGGTCTCGAATGGATTTCAACAATCTCTGCCGGT |
| DR594 | | GGCGACACCACTCTCTACGCGGATTCCGTGAAAGGGCGGTTCACCTCCTCTCGT |
| | | GACAATGCCAAGAACACTCTGTATCTCCAGCTTAACTCCTTGAAGACAGAGGAT |
| | | ACTGCCATCTATTACTGCGCCAAGCGCATCGACTGTAACTCTGGCTATTGTTAC |
| | | CGCCGTAACTACTGGGGACAGGGCACTCAAGTCACCGTCTCCTCTGGAGGCAGC |
| | | GGAGGTTCCGGGGGCTCTGGCCAGGTTCAGCTCCAGGAGAGCGGCGGAGGGAGC |
| | | GTGCAGGCAGGTGGAAGCCTGCGTCTGTCCTGTGTGGCCTCCGGTTCAACCTAC |
| | | TGCACCTATGATATGCACTGGTATCGTCAGGCCCCCGGCAAAGGTAGAGAGTTC |
| | | GTGTCCGCCATTGATTCCGATGGTACTACCAGATATGCCGACAGCGTAAAGGGA |
| | | CGTTTCACGATCTCTCAAGGCACCGCCAAGAACACCGTTTACCTCCAGATGAAT |
| | | AGCCTCCAGCCGGAAGATACTGCAATGTATTACTGCAAGACTGTTTGCGTGGTT |
| | | GGCAGCCGCTGGAGCGACTACTGGGGCCAGGGGACCCAGGTGACCGTCTCCTCT |
| hIL27 | 1323 | CAGGTCCAGTTGCAGGAATCCGGCGGAGGCCTGGTGCAGCCAGGGGGCTCTCTG |
| Ra_VH | | AGGCTGAGCTGTGCCGCGAGCGGCTTCACATTCTCCAGCTACCCCATGTCTTGG |
| H7- | | GTGCGTCAGGCCCCTGGCAAGGGTCTGGAGTGGATCTCCACCATCTCTGGTGGC |
| DR595 | | GGTGACACCACTCTGTATGCCGACAGTGTTAAAGGGCGTTTCACGTCTTCCCGT |
| | | GACAATGCCAAGAACACTCTCTACCTCCAGTTGAACTCACTGAAAACCGAGGAT |
| | | ACCGCCATTTATTACTGCGCCAAGCGTATCGACTGCAACTCTGGGTACTGTTAT |
| | | CGCCGGAACTACTGGGGCCAGGGGACCCAGGTGACTGTCAGTTCTGGGGGGGGC |
| | | TCTCAGGTTCAGCTCCAGGAGTCAGGAGGCGGTTCAGTGCAAGCTGGAGGCTCC |
| | | CTGACACTCAGCTGTGCCGCGAGTGAGTACGCATACTCCACCTGTAACATGGGT |
| | | TGGTATCGCCAGGCTCCAGGTAAAGAGCGCGAGCTGGTCTCCGCCTTCATCTCC |
| | | GACGGCTCCACGTATTACGCCGATTCTGTGAAAGGCCGTTTCACAATCACACGC |
| | | GACAATGCAAAGAACACAGTGTATGTTCAGATGAACTCACTCAAGCCTGAAGAC |
| | | ACCGCGATCTATTACTGCTCCGCAAACTGTTACAGACGCCTCCGCAACTACTGG |
| | | GGGCAAGGCACTCAAGTGACTGTTTCCAGT |
| hIL27 | 1324 | CAGGTACAGTTGCAGGAGTCAGGAGGTGGCCTGGTGCAGCCTGGTGGCAGCTTG |
| Ra_VH | | CGCCTGTCTTGCGCAGCCTCTGGCTTCACATTCAGCTCATACCCTATGTCTTGG |
| H7- | | GTCAGGCAGGCTCCAGGTAAGGGGCTGGAATGGATTTCTACCATTAGTGCAGGG |
| DR595 | | GGTGACACAACCCTGTACGCCGACTCAGTCAAGGGCCGGTTTACGTCCAGCCGC |
| | | GACAATGCAAAGAACACTCTCTACTTGCAGCTCAACAGTCTGAAGACCGAGGAT |
| | | ACTGCCATCTACTATTGTGCTAAGAGAATTGATTGCAACAGCGGCTATTGCTAC |
| | | CGCAGAAATTACTGGGGGCAGGGCACCCAGGTGACTGTTTCTTCCGGGGGCAGC |
| | | GGGGGTAGCGGTGGGTCCGGTCAGGTCCAGCTTCAGGAGTCCGGCGGAGGCAGC |
| | | GTGCAGGCCGGAGGCAGCCTCACCCTGTCTTGTGCCGCTTCTGAATACGCTTAT |
| | | TCTACTTGTAACATGGGCTGGTATCGGCAGGCTCCCGGTAAGGAGCGCGAGCTG |
| | | GTCAGCGCCTTCATCTCTGACGGATCTACTTATTACGCCGATTCCGTCAAGGGT |
| | | CGCTTCACCATCACCAGAGACAATGCCAAAAACACCGTCTATCTCCAGATGAAC |
| | | TCTCTCAAGCCGGAGGACACCGCGATTTACTATTGCCCTGCGAACTGTTACCGT |
| | | CGCCTCCGTAACTATTGGGGTCAGGGCACCCAGGTCACGGTGTCCAGC |
| hIL27 | 1325 | CAGGTACAACTCCAGGAGTCCGGGGGGGGACTGGTGCAGCCGGGTGGCTCCCTT |
| Ra_VH | | CGTCTGAGCTGTGCTGCCAGCGGTTTCACCTTCAGCTCCTACCCAATGTCTTGG |
| H7- | | GTCCGTCAAGCGCCTGGTAAGGGTCTTGAGTGGATTAGTACTATCAGCGCTGGT |
| DR596 | | GGCGATACCACGCTCTACGCTGATTCCGTCAAAGGCCGCTTTACTTCTTCCAGG |
| | | GATAACGCAAAAAACACTCTTTACCTCCAGCTGAACTCCCTGAAGACCGAAGAT |
| | | ACCGCAATCTATTACTGTGCTAAACGGATTGATTGCAACTCCGGCTACTGTTAT |
| | | CGCCGTAATTACTGGGGCCAGGGTACACAGGTGACCGTGTCCTCAGGGGGGGGC |
| | | TCCCAAGTTCAGCCTCAGGAGAGTGGGGGTGGCCTGGTCCAGCCGGGGGGTTCC |
| | | CTTCGCCTGTCCTGCACCGCGTCCGGCGTTACCTTCGACGATAGCGTGATGGGT |
| | | TGGTTCCGCCAGGCTCCCGGCAAGGGCAGGGAAGCCGTGAGCCGCATTTCATCC |
| | | TCAGGAGCCAACGCTTTCTACGCTGATTCTGTGAAGGGACGTTTTACTATCAGC |
| | | CGCGATAACGCCAAGAACACTGGTACCTCCAGATGAACTCCCCTGAAGCCTGAG |
| | | GATACGGCTACATATTACTGCAAGCGCGGTCACGCCCGCGCTGGATATTACCCG |
| | | ATCCCATACGATGACTACTGGGGCCAGGGAACACAGGTAACAGTCTCCTCT |
| hIL27 | 1326 | CAGGTTCAACTGCAAGAGTCCGGGGGGGGACTTGTCCAGCCGGGAGGCAGTCTG |
| Ra_VH | | AGATTGTCCTGCGCAGCGTCCGGGTTCACTTTCAGCTCTTATCCAATGTCATGG |
| H7- | | GTCCGTCAGGCCCCTGGCAAAGGGCTGGAGTGGATCAGCACCATTTCTGCTGGA |
| DR596 | | GGTGACACTACCCTGTACGCCGACAGCGTTAAGGGCCGCTTCACATCATCCAGG |
| | | GACAACGCCAAGAACACACTGTACCCTCAGCTGAACCCCCTGAAAACAGAGGAC |
| | | ACCGCTATCTATTACTGTGCGAAGCGCATTGACTGTAACAGCGGCTATTGTTAT |
| | | CGCAGGAACTATTGGGGACAGGGAACCCAGGTGACTGTAAGCTCCGGTGGGTCT |
| | | GGGGGCTCCGGCGGTTCTGGTCAGGTGCAACTCCAGGAGAGCGGGGGTGGCCTG |
| | | GTGCAGCCTGGCGGGAGTCTGAGGCTTTCCTGCACCGCCAGGGGCCTTACCTTC |
| | | GATGACTCCGTGATGGGGGGTTCCGCCAAGGGCCAGGAAAAGGCCGCGCAAGCC |
| | | GTCTCCTGTATCTCCTCAAGCGGCGCGAACGCGTTCTATGCCGACTCAGTGAAG |
| | | GGCCGTTTCACAATCTCCCGCGATAACGCCAAGAACACCCTGTATCTCCAGATG |
| | | AACAGCCTGAAGCCGGAAGACACGGCAACCTACTATTGTAAGAGAGGCCACGCT |
| | | TGCGGTGGGTATTACCCTATCCCTTACGACGATTACTGGGGACAGGGTACTCAG |
| | | GTGACCGTTAGCTCT |
| hIL27 | 1327 | CAAGTTCAGCTTCAGGAGTCAGGTGGCGGATCAGTGCAAGTCGGAGGCTCCCTG |
| Ra_VH | | CGCCTGTCTTGCGCTGCCTCCGGCTTTACATTCTCTTCCTACCCCATGTCCTGG |
| H8- | | GTAAGGCAGGCTCCCGGCAAAGGCCTGGAGTGGATTAGCACAATCTCTGCCGGT |
| DR591- | | GGCGACACCACTCTGTACGCTGATTCCGTGAAAGGTCGTTTCACGTCTAGCAGA |
| | | GACAACGCTAAGAACACCCTCTACCCTCAGCTCAACAGCCTGAAGACGGAGGAC |
| | | ACCGCTATTTATTACTGTGCGAAGCGCATCGACTGTAACTCTGGATACTGCTAT |
| | | CGGCGCAACTATTGGGGTCAGGGTACTCAGGTTACAGTGTCATCCGGGGGGGGA |
| | | TCACAAGTGCAACTGCAAGAATCCGGGGGGGGTAGCGTGCAGGCCGGTGGCTCA |
| | | TTGCGTCTGTCCTGCACCGCCTCTGGCGCTATCGCTTCTGGCTATATCGACAGT |
| | | CGGTGGTGCATGGCCTGGTTCCGCCAGGCCCCTGGGAAGGAGCGCGAGGGTGTG |
| | | GCCGCGATCTGGCCCGGTGGGGGCCTCACAGTGTATGCAGACTCCGTAAAAGGC |
| | | CGCTTCACAATTAGCCGCGACCACGCTAAGAACACCCTTTACTTGCAGATGAAC |
| | | AATCTGAAGCCAGAAGACACTGCAATGTACTATTGTGCTGCGGGTTCCCCTCGT |
| | | ATGTGCCCAAGTCCTGAGTTCGGTTTTGATTACTGGGGGCAGGGCACCCAGGTC |
| | | ACCGTGTCCAGT |
| hIL27 | 1328 | CAGGTACAACTCCAGGAATCTGGCGGAGGCTCCGTTCAGGCGGGGGCGTCTCTG |
| Ra_VH | | AGACTGAGCTGCGCTGCCAGCGGATTTACTTTCAGCTCCTACCCCATGAGTTGG |
| H8- | | GTGAGGCAGGCTCCCGGTAAGGGCCTGGAATGGATTAGCACTATTAGCGCTGGC |
| DR591 | | GGGGACACCACTCTGTACGCTGACAGTGTCAAGGGGCGGTTTACTAGCTCCCGC |
| | | GACAACGCCAAGAACACTCTGTATCTCCAGCTCAACAGCCTCAAAACTGAGGAC |
| | | ACAGCTATCTATTACTGTGCTAAGCGCATTGACTGTAACTCAGGCTATTGTTAC |
| | | CGCCGGAACTACTGGGGACAAGGGACCCAAGTGACAGTTTCCTCTGGGGGTAGC |
| | | GGCGGATCTGGGGGCTCTGGCCAGGTCCAGCTTCAGGAGTCCGGGGGAGGCTCC |
| | | GTCCAGGCCGGTGGCTCTTTGAGGCTGTCATGCACAGCTTCCGGTGCCATCGCA |
| | | AGTGGATATATTGACAGTCGCTGGTGCATGGCTTGGTTCCGGCAAGCCCCCGGA |
| | | AAGGAGAGAGAGGGCGTTGCTGCAATCTGGCCGGGTGGGGGCTTGACCGTCTAT |
| | | GCTGATTCTGTGAAGGGCCGTTTTACCATCAGTAGAGACCACGCTAAAAACACC |
| | | CTGTACTTGCAGATGAATAACTTGAAACCCGAAGACACCGCCATGTATTACTGC |
| | | GCCGCTGGCTCTCCCCGTATGTGCCCCAGCTTGGAGTTCGGCTTTGATTATTGG |
| | | GGTCAGGGAACTCAGGTCACTGTGTCCAGC |
| hIL27 | 1329 | CAGGTGCAGCTCCAGGAGTCTGGCGGTGGAAGCGTGCAAGTAGGAGGCTCTCTG |
| Ra_VH | | AGGCTGTCATGCGCAGCGAGCGGCTTCACCTTCAGCTCTTATCCAATGTCTTGG |
| H8- | | GTTCGCCAGGCCCCCGGTAAGGGTCTGGAGTGGATCTCAACTATCAGGGCAGGC |
| DR592 | | GGGGATACAACCCTTTACGCCGATAGTGTGAAGGGACGCTTTACCTCTAGCAGA |
| | | GATAACGCTAAGAACACTCTGTACTTGCAGCTTAACTCCCTTAAAACCGAGGAC |
| | | ACCGCGATCTATTACTGTGCCAAGCGCATTGACTGCAACTCTGGTTACTGCTAT |
| | | AGAAGGAACTACTGGGGCCAGGGTACGCAGGTGACGGTTAGTTCCGGTGGAGGC |
| | | TCACAAGTGCAGCTTCAGGAGTCCGGGGGTGGGTCTGTGCAGGCTGGCGGGTCC |
| | | CTGCGGCTTTCCTGCACAGCCCCCGGCTTCACGTCTAACTCCTGTGGCATGGAC |
| | | TGGTATCGCCAGGCCCCTGGTAAAGAGCGTGAGTTCGTGAGCAGTATTTCTACC |
| | | GATGGAACGACCGGCTATGCTGACTCCGTGAAGGGTCGGTTCACCATCTCCAAG |
| | | GACAAAGCCAAAGACACCGTTTATTTGCAGATGAACTCCCTCAAGCCAGAGGAC |
| | | ACTGGCATGTATAGTTGCAAGACCAAGGACGGCACTATTGCGACGATGGAGCTG |
| | | TGTGACTTCGGCTACTGGGGCCAGGGAACCCAGGTGACGGTGTCTTCT |
| hIL27 | 1330 | CAGGTGCAGGTCCAAGAGTCTGGTGGGGGAAGCGTTCAAGTCGGGGGCAGCCTG |
| Ra_VH | | AGACTGTCCTGTGCAGCTTCTGGCTTCACCTTTTCTAGCTACCCTATGCCCTGG |
| H8- | | GTGCGCCAGGCCCCCGGCAAAGGCCTGGAATGGATCTCCACCATCAGTGGGGGA |
| DR592 | | GGTGACACAACTCTGTACGCGGATTCTGTGAAGGGCCGGTTCACATCTAGCCGT |
| | | GATAACGCCAAGAATACTTTGTATCTCCAGCTCAACTCTCTGAAGACTGAAGAT |
| | | ACAGCAATTTATTACTGCGCAAAACGGATTGATTGTAATAGGGGGTACTGTTAC |
| | | CGCAGGAACTACTGGGGTCAAGGCACTCAGGTTACGGTGTCTTCCGGGGGATCT |
| | | GGAGGTAGCGGTGGCAGCGGTCAGGTGCAGCTCCAGGAGTCAGGGGGGGGTTCC |
| | | GTGCAGGCTGGCGGGTCCCTGCGCCTCTCTTGTACTGCCCCTGGGTTTACATCC |
| | | AACTCCTGTGGTATGGACTGGTATAGGCAAGCTCCGGGAAAGGAGCGCGAGTTC |
| | | GTGTCTTCCATCAGCACCGATGGCACCACTGGTTACGCCGATTCCGTGAAGGGA |
| | | CGCTTTACAATTAGCAAGGACAAGGCCAAGGATACTGTGTACTTGCAGATGAAC |
| | | AGCCTGAAGCCTGAGGACACTGGGATGTACTCTTGCAAGACCAAGGACGGGACC |
| | | ATCGCCACGATGGAACTCTGCGACTTCGGATACTGGGGCCAGGGCACGCAAGTG |
| | | ACCGTAAGCTCT |
| hIL27 | 1331 | CAGGTACAGCTCCAGGAGAGCGGCGGTGGCTCCGTCCAAGTAGGGGGCAGCCTC |
| Ra_VH | | CGCCTTTCTTGTGCAGCCTCTGGTTTTACATTTTCCAGGTACCCAATGAGCTGG |
| H8- | | GTCCGGCAAGCGCCTGGGAAAGGTCTGGAGTGGATTTCCACCATCTCCGGGGGG |
| DR593 | | GGCGACACTACCCTGTACGCAGATAGCGTAAAGGGTCGCTTTACCAGTTCTCGT |
| | | GATAACGCCAAGAACACTCTGTACCTTCAGCTGAATAGCCTCAAGACCGAGGAT |
| | | ACCGCCATCTACTATTGCGCTAAGCGCATCGACTGCAACTCTGGCTATTGTTAC |
| | | AGACGTAACTACTGGGGTCAAGGTACGCAGGTAACTGTATCCTCTGGAGGCGGT |
| | | TCTCAGGTACAGCTTCAGGAGAGCGGCGGTGGCAGCGTGCAGGCCGGAGGGTCC |
| | | CTGCGTCTCTCCTGCGCTGCCTCCGGCTACCCCTACTCCAATGGCTATATGGGT |
| | | TGGTTCAGACAGGCTCCTGGCAAAGAACGGGAGGGGGTGGCCACAATTTACACC |
| | | GGTGATGGCCGCACGTACTATGCCGACTCCGTGAAGGGGCGCTTTACCATTTCT |
| | | CGCGATAACGCTAAAAACACCGTGGACCTCCAGATGTCCAGCCTGAAGCCTGAA |
| | | GATACTGCAATGTATTACTGTGCCGCACGTGCTGCCCCACTGTACTCAAGTGGG |
| | | AGCCCACTGACTCGCGCTAGATATAACGTGTGGGGTCAGGGCACTCAGGTGACC |
| | | GTCTCCTCC |
| hIL27 | 1332 | CAAGTACAACTCCAGGAATCTGGGGGGGGGAGTGTTCAGGTCGGTGGCAGCCTG |
| Ra_VH | | CGTCTCAGTTGCGCCGCGTCTGGCTTCACATTTTCTTCCTATCCGATGTCTTGG |
| H8- | | GTGAGGCAAGCCCCTGGGAAGGGTCTCGAATGGATCTCCACAATGTCTGCTGGC |
| DR593 | | GGTGATACCACGCTCTACGCCGACTCTGTCAAAGGCCGCTTCACTTCTTCACGT |
| | | GATAACGCCAAAAACACCCTGTACCTTCAGCTCAACTCCCTGAAGACCGAAGAC |
| | | ACAGCAATCTATTACTGTGCGAAGCGGATTGATTGCAACTCCGGCTATTGTTAT |
| | | CGGAGGAACTACTGGGGCCAGGGCACCCAAGTGACCGTCTCCTCTGGCGGTTCC |
| | | GGTGGCTCCGGGGGTTCTGGACAAGTCCAACTCCAGGAGAGCGGCGGAGGCTCC |
| | | GTTCAGGCAGGCGGGTCCTTGAGGCTGAGTTGTGCTGCATCAGGCTACCCCTAC |
| | | TCAAACGGCTATATGGGCTGGTTCAGACAGGCTCCGGGCAAGGAGCGCGAGGGA |
| | | GTGGCCACTATCTATACCGGCGACGGTCGCACTTATTACGCAGATAGCGTAAAG |
| | | GGTCGCTTCACCATCAGCCGCGATAACGCCAAGAACACGGTTGACCTTCAAATG |
| | | TCTAGTCTCAAACCTGAAGACACTGCCATGTACTATTGCGCCGCAAGAGGTGCC |
| | | CCTCTGTATTCCAGCGGGAGCCCCCTGACCCGTGCCCGCTACAACGTGTGGGGT |
| | | CAGGGCACCCAGGTGACTGTCAGCTCT |
| hIL27 | 1333 | CAGGTGCAACTCCAGGAGAGTGGCGGGGGCTCCGTACAGGTCGGGGGCTCTCTG |
| Ra_VH | | AGACTCTCCTGTGCGGCCAGCGGATTTACCTTCAGCAGTTACCCGATGTCTTGG |
| H8- | | GTGCGTCAGGCCCCAGGCAAGGGCCTGGAGTGGATTTCCACAATCAGCGGTGGC |
| DR594 | | GGTGACACTACACTCTACGCAGATTCCGTGAAAGGGCGTTTCACCTCTAGCCGC |
| | | GACAACGCTAAGAACACCCTGTACTTGCAGTTGAACTCCCTGAAGACCGAGGAC |
| | | ACGGCCATCTACTATTGCGCGAAGCGCATCGACTGTAACAGCGGATACTGTTAC |
| | | AGGCGCAACTATTGGGGACAGGGGACCCAAGTAACCGTGAGTTCAGGTGGGGGC |
| | | TCCCAGGTGCAGTTGCAGGAGTCAGGTGGAGGCAGCGTCCAGGCCGGGGGCTCC |
| | | CTTCGCCTGTCCTGTGTGGCCAGCGCCTCTACTTACTGTACGTATGATATGCAC |
| | | TGGTATAGACAGGCCCCCGGTAAGGGAAGAGAGTTCGTCAGCGCTATTGATAGC |
| | | GATGGCACCACACGCTATGCGGATTCCGTAAAGGGTAGGTTTACAATCAGCCAG |
| | | GGAACCGCAAAAAACACTGTTTACTTGCAGATGAACAGCCTCCAGCCAGAGGAT |
| | | ACCGCCATGTACTATTGTAAGACTGTGTGTGTCGTGGGTTCCCGTTGGTCCGAT |
| | | TACTGGGGCCAGGGCACTCAAGTAACAGTTAGTAGT |
| hIL27 | 1334 | CAAGTTCAACTGCAAGAGTCCGGTGGCGGAAGCGTCCAGGTGGGAGGCTCCCTT |
| Ra_VH | | CGCCTGAGCTGTGCTGCCAGCGGTTTCACATTCAGTTCCTATCCGATGTCCTGG |
| H8- | | GTGCGTCAGGCCCCAGGCAAGGGTCTGGAGTGGATCTCTACCATCTCCGCTGGC |
| DR594 | | GGTGACACGACTCTGTACGCTGATAGCGTTAAGGGGCGCTTTACCTCCCCTCGC |
| | | GATAACGCTAAGAACACTCTCTACCTCCAGTTGAATAGCCTCAAGACTGAAGAT |
| | | ACCGCTATCTACTATTGCGCTAAGCGGATTGATTGCAACTCTGGGTACTGCTAC |
| | | CGCCGGAACTACTGGGGACAGGGAACCCAGGTGACTGTGTCCTCTGGTGGCTCT |
| | | GGAGGCTCCGGCGGTTCCGGGCAGGTCCAACTCCAGGAATCCGGGGGTGGCAGC |
| | | GTGCAGGCCGGTGGCTCCTTGCGTCTGTCCTGCGTGGCGAGCGCTTCCACCTAC |
| | | TGCACCTACGACATGCACTGGTATCGCCAGGCTCCGGGTAAGGGCCGTGAATTT |
| | | GTGTCTGCCATTGATTCTGACGGCACCACGCGGTACGCAGACTCTGTTAAGGGG |
| | | CGCTTTACCATCAGTCAGGGCACGGCTAAGAACACCGTGTACCTGCAAATGAAC |
| | | TCTCTCCAACCGGAGGACACTGCCATGTATTACTGTAAAACGGTTTGTGTTGTC |
| | | GGCTCCCGCTGGTCCGATTACTGGGGCCAGGGAACCCAGGTGACAGTCTCAAGC |
| hIL27 | 1335 | CAGGTTCAACTCCAGGAGAGTGGCGGTGGCTCAGTGCAAGTAGGGGGCAGTCTG |
| Ra_VH | | CGCCTGAGCTGTGCTGCATCCGGCTTCACCTTCTCTAGTTATCCCATGAGCTGG |
| H8- | | GTACGTCAGGCTCCAGGCAAAGGTCTGGAGTGGATCAGCACCATTAGCGGTGGC |
| DR595 | | GGAGACACGACTCTCTATGCCGACAGCGTGAAGGGCCGGTTCACCTCCAGTCGG |
| | | GACAACGCCAAGAACACGCTGTACCTTCAGTTGAACTCACTCAAGACTGAGGAT |
| | | ACCGCAATCTATTACTGCGCTAAACGCATCGACTGTAACTCCGGGTACTGTTAC |
| | | CGTCGCAACTACTGGGGTCAGGGGACTCAGGTCACCGTGTCTTCCGGGGGTGGC |
| | | AGTCAGGTGCAGCTTCAGGAATCCGGGGGAGGCTCTGTCCAGGCTGGGGGTTCC |
| | | CTGACTCTGTCTTGCGCAGCCTCAGAGTATGCCTACAGCACTTGTAACATGGGT |
| | | TGGTATCGCCAGGCTCCCGGCAAAGAACGTGAGCTTGTTAGCGCGTTCATCAGC |
| | | GATGGCAGCACCTATTACGCTGACAGCGTGAAGGGTCGCTTTACAATCACCAGG |
| | | GACAACGCGAAGAACACCGTGTATCTCCAGATGAACTCCCTCAAGCCCGAGGAC |
| | | ACCGCCATCTATTACTGCTCCGCTAATTGCTACCGTCGGCTGCGCAACTACTGG |
| | | GGCCAGGGGACCCAGGTAACAGTCTCCTCT |
| hIL27 | 1336 | CAGGTCCAGGTCCAGGAGTCCGGTGGGGGAAGCGTCCAAGTAGGGGGATCTCTG |
| Ra_VH | | AGGCTGTCCTGTGCGGCCTCCGGCTTCACCTTCTCCAGCTACCCCATGTCCTGG |
| H8- | | GTGAGACAAGCCCCTGGGAAAGGACTGGAATGGATTAGTACCATTTCAGCCGGG |
| DR595 | | GGTGACACAACCCTGTACGCAGACTCCGTGAAAGGACGTTTTACCAGTTCCCGT |
| | | GATAACGCCAAGAACACCTTGTACCTCCAGTTGAACTCCCTGAAGACAGAAGAC |
| | | ACAGCCATCTATTACTGTGCCAAGCGCATTGATTGCAACAGCGGATACTGTTAC |
| | | CGCAGAAACTATTGGGGCCAGGGCACCCAGGTGACAGTATCAAGCGGCGGTTCC |
| | | GGCGGTAGCGGCGGTAGCGGGCAAGTGCAGTTGCAGGAGAGGGGGGGTGGCTCC |
| | | GTTCAGGCAGGAGGCTCCCTGACTCTCTCCTGTGCTGCCTCTGAGTATGGGTAC |
| | | TCCACTTGCAATATGGGCTGGTACAGGCAGGGTCCCGGCAAGGAACGCGAACTG |
| | | GTGAGTGCATTCATCTCTGATGGCTCCACATATTACGCCGACAGCGTCAAGGGC |
| | | CGGTTTACCATCACTCGGGATAATGCCAAGAACACCGTCTACCTGCAGATGAAC |
| | | AGCCTGAAGCCAGAGGACACAGCCATCTATTACTGCTCTGCTAACTGCTACCGC |
| | | AGACTGCGCAATTACTGGGGCCAGGGAACCCAGGTGACTGTGTCTAGC |
| hIL27 | 1337 | CAGGTGCAACTCCAGGAATCCGGCGGAGGCAGGGTGCAAGTTGGGGGCAGTCTT |
| Ra_VH | | CGTCTGTCATGTGCAGCCAGCGGGTTCACCTTCTCAAGCTACCCGATGAGCTGG |
| H8- | | GTGCGGCAGGCTCCCGGAAAGGGCCTGGAGTGGATTTCCACTATCTCCGCTGGC |
| DR596 | | GGTGACACCACACTGTACGCTGACTCCGTGAAGGGGCGGTTCACGTCATCCCGT |
| | | GATAACGCTAAGAACACACTGTATCTCCAGCTGAACAGCCTCAAGACCGAGGAC |
| | | ACTGCAATCTACTATTGCGCTAAGAGAATTGATTGCAACTCCGGGTACTGTTAC |
| | | AGGCGTAATTACTGGGGGCAGGGCACACAAGTGACCGTGTCCAGTGGGGGTGGC |
| | | AGCCAGGTGCAGTTGCAGGAGAGTGGCGGGTGGCCTTGTGCAGCCCGGGGGAGT |
| | | CTGCGCCTGTCCTGCACAGCCTCCGGGCTGACCTTCGACGATTCTGTAATGGGC |
| | | TGGTTCCGCCAGGCTGCGGGGAAGGGCCGCGAGGCCGTCAGCTGTATCTCCAGC |
| | | TCTGGAGCCAACGCATTTTACGCGGACAGCGTAAAGGGTAGATTCACAATTTCA |
| | | CGTGACAACGCCAAGAACACCCTGTACCTCCAGATGAACAGCCTGAAGCCCGAG |
| | | GATACCGCCACCTACTATTGTAAGCGCGGCCACGCTTGCGCGGGTTACTATCCG |
| | | ATCCCATACGATGACTATTGGGGCCAGGGCACGCAGGTTACGGTGTCTTCC |
| hIL27 | 1338 | CAGGTCCAACTGCAAGAGTCTGGCGGGGGTTCCGTTCAGGTCGGTGGCTCCTTG |
| Ra_VH | | CGGCTGAGCTGTGCTGCCTCCGGCTTTACTTTTAGTTCTTATCCGATGAGCTGG |
| H8- | | GTGAGGCAGGCTCCTGGCAAGGGCCTGGAGTGGATCTCCACAATCAGCGGTGGC |
| DR596 | | GGGGACACCACATTGTACGCCGACTCCGTCAAGGGCAGGTTCACCTCCTCACGC |
| | | GATAATGCTAAGAACACTCTGTATCTCCAGCTTAACTCTCTCAAGACCGAGGAC |
| | | ACCGCCATTTATTACTGCGCGAAGAGAATTGATTGTAACTCCGGCTACTGCTAC |
| | | CGCCGTAATTACTGGGGTCAGGGAACCCAGGTTACGGTCAGCTCCGGTGGCAGT |
| | | GGGGGTAGCGGAGGCTCCGGCCAGGTGCAGTTGCAGGAGTCTGGCGGTGGCCTG |
| | | GTCCAGCCTGGAGGCAGCCTCCGGCTGAGCTGTACTGGTTCAGGCCTGACATTT |
| | | GACGATAGCGTGATGGGGGGTTCCGCCAAGCGCCGGGAAAGCGGTCGCGAGGCC |
| | | GTCTCTTGTATCAGTTCCTCTGGAGCCAACGCTTTCTATGCTGATAGCGTGAAG |
| | | GGCCGCTTCACAATTAGCCGGGACAATGCAAAGAACACCTTGTACCTGCAAATG |
| | | AACTCTCTGAAGCCCGAAGATACGGCTACCTACTATTGCAAGCGCGGCCATGCC |
| | | TGCGCAGGATATTACCCAATCCCCTACGACGATTATTGGGGTCAGGGCACCCAG |
| | | GTGACCGTGAGCAGG |
| hIL27 | 1339 | CAGGTCCAACTCCAGGAGAGCGGGGGTGGCAGTGTGCAGTCTGGCGGTTCCCTT |
| Ra_VH | | AGGCTGAGTTGCGCAGCCAGCGGCTTCACATACAGCACCAGCAATTCATGGATG |
| H9- | | GCGTGGTTCCGTCAGGCCCCCGGTAAGGAGCGGGAGGGCGTCGCCGCTATCTAG |
| DR591 | | ACAGTGGGGGGTCCATCTTTTCACGCTGATTCCGTGCGTGGCCGTTTCACCATC |
| | | TCCCAAGATGCGACAAAGAATATGTTCTACCTGCAAATGAACACACTCAAGCCC |
| | | GAAGATACCGCTATGTATTACTGCGCGGCAGCTTCTGGCCGCCTCCGTGGAAAG |
| | | TGGTTCTGGCCATACGAATATAACTATTGGGGCCAGGGAACTCAGGTGACAGTT |
| | | AGTAGCGGCGGTGGCTCTCAGGTGCAACTCCAGGAGTCCGGGGGGGGAAGCGTG |
| | | CAGGCGGGAGGGAGCCTGCGGCTGTCATGTACCGCCAGCGGAGCCATCGCCAGC |
| | | GGATACATTGATTCCCGCTGGTGTATGGCTTGGTTTCGCCAGGCCCCCGGCAAG |
| | | GAAAGGGAGGGAGTCGCCGCTATTTGGCCCGGTGGCGGTCTGACAGTGTACGCC |
| | | GACAGCGTAAAGGGTCGCTTCACTATCAGCCGTGACCACGCTAAGAACACCCTG |
| | | TACCTCCAGATGAATAACTTGAAGCCAGAGGACACCGCCATGTACTATTGCGCC |
| | | GCTGGCTCCCCGAGAATGTGCCCTTCTCTCGAATTTGGTTTTGACTACTGGGGG |
| | | CAGGGGACCCAGGTCACGGTTTCTTCC |
| hIL27 | 1340 | CAGGTGCAACTCCAGGAGAGTGGGGGGGGTTCCGTCCAGAGCGGTGGCAGTCTG |
| Ra_VH | | AGGCTGTCCTGCGCCGCTTCCGGTTTCACATACTCCACCAGTAACAGCTGGATG |
| H9- | | GCCTGGTTTCGCCAAGCCCCTGGGAAAGAGAGAGAAGGAGTCGCCGCAATCTAC |
| DR591 | | ACTGTAGGAGGCTCCATTTTCTACGCAGACTCTGTCAGAGGCCGTTTCACCATT |
| | | AGCCAGGATGCCACTAAGAATATGTTTTACCTCCAGATGAATACACTGAAGCCA |
| | | GAAGACACCGCTATGTATTACTGTGCTGCGGCTTCCGGCAGGCTGCGCGGCAAG |
| | | TGGTTCTGGCCTTACGAGTATAACTATTGGGGCCAAGGCACCCAGGTCACCGTG |
| | | TCAAGCGGGGGTTCCGGTGGCAGCGGAGGGAGCGGCCAGGTGCAGCTGCAAGAG |
| | | TCTGGTGGCGGTTCCGTCCAGGCTGGGGGCTCTCTCCGGCTGTCTTGTACGGCC |
| | | AGCGGTGCCATCGCTAGTGGCTACATCGACAGGGGCCGGGGCATGGCCTGGTTC |
| | | CGTCAGGCTCCTGGAAAGGAACGGGAAGGCGTCGCCGCTATTTGGCCAGGTGGC |
| | | GGACTGACGGTCTATGCTGACTCTGTCAAGGGACGCTTCACCATCAGTCGCGAT |
| | | CACGGTAAGAACACCGTCTACCTCCAGATGAATAACCTGAAGCCCGAGGATACG |
| | | GCCATGTATTACTGCGCCGCTGGTTCCCCAAGAATGTGTCCCAGCCTGGAGTTC |
| | | GGGTTCGACTATTGGGGACAGGGCACCCAGGTAACCGTGTCATCA |
| hIL27 | 1341 | CAGGTCCAGCTCCAGGAGAGGGGAGGCGGTAGTGTCCAGTCCGGTGGCTCCCTG |
| Ra_VH | | CGTCTGAGTTGCGCAGCCAGTGGCTTCACTTATTCCACAAGCAACTCTTGGATG |
| H9- | | GCATGGTTTCGCCAGGCCCCTGGGAAGGAACGCGAGGGTGTCGCTGCCATCTAC |
| DR592 | | ACAGTGGGCGGTTCCATTTTCTACGCAGACTCCGTTAGAGGTAGGTTTACTATC |
| | | AGCCAGGATGCTACCAAGAATATGTTCTACCTTCAGATGAACACACTCAAGCCC |
| | | GAAGACACCGCCATGTATTACTGTGCCGCAGCCTCTGGCCGCCTGCGCGGTAAG |
| | | TGGTTCTGGCCTTACGAGTACAATTACTGGGGTCAGGGCACCCAGGTGACAGTG |
| | | TCCAGTGGTGGAGGCTCCCAGGTTCAGCTTCAGGAGTCTGGAGGGGGCTCCGTG |
| | | CAGGCAGGAGGCTCTCTGAGACTCAGCTGCACCGCCCCAGGTTTTACCTCCAAC |
| | | AGCTGCGGCATGGACTGGTATCGCCAGGCCCCCGGCAAGGAACGCGAGTTCGTG |
| | | AGTTCTATCTCCACCGATGGAACAACGGGTTACGCCGATTCTGTGAAGGGCCGG |
| | | TTTACAATCTCCAAGGATAAGGCCAAGGACACTGTGTACCTCCAGATGAACTCC |
| | | TTGAAACCAGAAGACACAGGCATGTATAGCTGTAAGACCAAGGATGGGACCATC |
| | | GCGACTATGGAACTGTGTGACTTCGGATACTGGGGCCAGGGGACCCAGGTCACA |
| | | GTGTCCTCC |
| hIL27 | 1342 | CAGGTCCAGCTGCAAGAGAGCGGAGGGGGTAGTGTGCAGAGCGGCGGAAGCCTG |
| Ra_VH | | CGCCTCAGCTGCGCCGCGTCCGGCTTTACCTACAGCACAAGTAACTCTTGGATG |
| H9- | | GCCTGGTTCCGGCAGGCTCCCGGCAAGGAAAGGGAAGGGGTGGCCGCAATCTAC |
| DR592 | | ACGGTCGGAGGTTCTATTTTCTACGCCGATAGCGTCAGAGGCCGGTTCACAATC |
| | | TCTCAGGACGCAACCAAAAATATGTTCTACCTGCAGATGAACACACTCAAGCCC |
| | | GAGGACACCGCGATGTATTACTGTGCCGCTGCATCCGGGGGGCTGAGAGGAAAG |
| | | TGGTTCTGGCCTTATGAGTACAATTATTGGGGTCAGGGCACTCAGGTGACTGTG |
| | | TCCTCTGGTGGCTCCGGTGGCAGTGGGGGCAGCGGTCAGGTGCAGCTTCAGGAG |
| | | TCCGGTGGAGGGAGCGTCCAGGCTGGCGGTTCCCTCAGGGTGTCCTGCACAGCA |
| | | CCGGGCTTCACTAGCAACAGCTGCGGTATGGATTGGTATCGCCAGGCACCGGGT |
| | | AAGGAGCGCGAATTTGTCTCATCTATCAGCACCGATGGGACAACCGGGTACGCT |
| | | GATAGCGTGAAAGGTAGGTTCACCATCTCCAAGGACAAGGCAAAAGATACCGTG |
| | | TACCTCCAGATGAACTCTCTCAAGCCCGAGGACACCGGAATGTATAGCTGCAAG |
| | | ACAAAGGACGGCACCATTGCAACAATGGAGCTTTGTGACTTTGGCTATTGGGGC |
| | | CAGGGCACCCAGGTAACGGTCTCTTCA |
| hIL27 | 1343 | CAGGTCCAGCTTCAAGAATCCGGGGGGGGCTCCGTACAATCAGGAGGGTCACTC |
| Ra_VH | | AGGCTGAGTTGCGCAGCCTCTGGATTCACGTACTCCACAAGCAATTCCTGGATG |
| H9- | | GCCTGGTTTAGACAGGCACCCGGCAAGGAGCGTGAGGGGGTGGCTGCCATCTAC |
| DR593 | | ACTGTGGGGGCTCCATCTTCTATGCGGACAGTGTTCGCGGCGCGTTTTACAATC |
| | | TCCCAGGACGCCACCAAGAATATGTTCTACTTGCAGATGAACACACTGAAACCG |
| | | GAGGACACCGCCATGTATTACTGTGCAGCCGCGTCAGGCCGCCTGCGGGGGAAG |
| | | TGGTTCTGGCCATACGAGTACAACTACTGGGGTCAGGGCACCCAAGTGACCGTG |
| | | AGTTCCGGGGGAGGTTCCCAGGTTCAGCTTCAGGAGTCCGGTGGGGGGTCTGTG |
| | | CAGGCCGGTGGCTCCCTTCGGCTCTCCTGCGCCGCGAGTGGCTATCCTTATTCC |
| | | AACGGCTACATGGGCTGGTTCCGTCAGGCACCCGGCAAGGAACGGGAAGGCGTC |
| | | GCTACCATCTATACAGGTGATGGGCGGACTTATTACGCAGACTCCGTGAAGGGC |
| | | CGCTTTACTATCAGTCGCGACAACGCAAAGAATACAGTGGACTTGCAAATGAGT |
| | | TCCCTCAAGCCTGAGGACACTGCTATGTACTATTGTGCTGCCCGTGCAGCGCCA |
| | | CTGTATAGTTCCGGCTCACCGCTGACCCGCGCTAGGTATAACGTATGGGGTCAG |
| | | GGCACACAAGTGACGGTGTCAAGC |
| hIL27 | 1344 | CAGGTGCAACTCCAGGAGTCCGGGGGAGGCAGTGTGCAATCCGGGGGTTCTCTG |
| Ra_VH | | CGCCTCTCTTGCGCTGCCTCTGGGTTTACATATTCCACATCCAACTCCTGGATG |
| H9- | | GCCTGGTTCCGCCAGGCTCCCGGCAAGGAGAGAGAGGGGCGGGGCGCGATCTAC |
| DR593 | | ACCGTTGGTGGCTCTATCTTCTATGCCGACAGCGTTCGGGGCCGCTTTACCATT |
| | | AGCCAGGATGCAACTAAGAATATGTTCTACCTTCAGATGAACACACTGAAGCCA |
| | | GAGGATACTGCAATGTATTACTGCGCCGCAGCTTCTGGCAGACTGCGGGGAAAA |
| | | TGGTTCTGGCCTTATGAGTACAATTATTGGGGGCAGGGCACCCAGGTCACAGTA |
| | | AGCTCCGGTGGCTCCGGCGGTAGCGGAGGCTCCGGCCAAGTCCAACTTCAGGAA |
| | | AGCGGTGGGGGGTCTGTGCAGGCCGGTGGCTCCCTCCGCCTGTCTTGCGCGGCT |
| | | TCCGGCTATCCATACAGTAACGGCTACATGGGTTGGTTCCGGCAGGCCCCTGGC |
| | | AAGGAGCGTGAGGGCGTTGCTACGATCTATACAGGTGACGGAAGGACTTACTAT |
| | | GCTGACAGCGTAAAGGGGCGCTTCACCATTTCTCGTGACAACGCAAAGAACACA |
| | | GTGGACCTCCAGATGAGCAGTCTGAAGCCTGAGGACACCGCGATGTACTATTGT |
| | | GCCGGTCGGGCCGCTCCTCTGTATTCCAGCGGCAGCCCTCTGACTAGGGCTAGA |
| | | TACAACGTCTGGGGACAGGGCACTCAGGTGACTGTGAGCAGC |
| hIL27 | 1345 | CAGGTGCAACTCCAGGAGTCTGGAGGGGGCTCCGTGCAGAGTGGCGGAAGCCTG |
| Ra_VH | | AGGTTGTCCTGTGCCGCGAGTGGATTCACCTACTCCACCTCCAACAGCTGGATG |
| H9- | | GCCTGGTTTAGACAGGCTCCAGGTAAGGAAAGAGAGGGCGTGGCCGCGATCTAC |
| DR594 | | ACCGTAGGGGGCTCCATCTTCTATGCAGATAGCGTGAGGGGCCGGTTCACAATC |
| | | AGTCAGGATGCCACCAAAAATATGTTTTACCTCCAGATGAACACATTGAAGCCA |
| | | GAGGACACCGCCATGTACTATTGCGCCGCTGCCTCTGGTAGGCTGAGGGGCAAA |
| | | TGGTTCTGGCCCTATGAATATAACTACTGGGGGCAGGGCACCCAGGTAACGGTG |
| | | TCAAGCGGTGGGGGCTCTCAGGTGCAGCTCCAGGAGTCCGGGGGGGGCAGCGTA |
| | | CAGGCTGGAGGTAGTCTGCGCTTGAGTTGCGTCGCGTCAGCCTCTACCTATTGC |
| | | ACATACGACATGCACTGGTATAGACAGGCCCCAGGCAAGGGAAGGGAGTTCGTG |
| | | TCCGCAATCGACTCTGATGGCACAACCAGATACGCAGACTCCGTAAAGGGTCGC |
| | | TTCACCATCAGCCAGGGCACAGCGAAGAACACAGTGTACCTCCAGATGAACTCC |
| | | TTGCAGCCTGAGGATACTGCCATGTACTATTGCAAGACTGTGTGCGTTGTCGGA |
| | | TCTCGCTGGTCAGACTACTGGGGCCAGGGAACACAGGTGACAGTGTCCTCC |
| hIL27 | 1346 | CAGGTGCAACTCCAGGAGTCTGGAGGTGGCTCTGTCCAAAGCGGTGGCTCCCTT |
| Ra_VH | | CGCCTGAGCTGTGCCGCTAGTGGATTCACGTACTCTACGTCAAATAGCTGGATG |
| H9- | | GCGTGGTTCAGACAGGCCCCTGGCAAGGAGAGGGAGGGTGTGGCTGCGATCTAT |
| DR594 | | ACTGTGGGCGGTTCCATCTTCTATGCCGATTCCGTGCGCGGTCGGTTTACCATC |
| | | TCTCAGGACGCTACCAAGAATATGTTTTACTTGCAGATGAACACTCTGAAGCCA |
| | | GAGGATACCGCGATGTACTATTGCGCAGCGGCCTCTGGTAGGCTGAGAGGCAAA |
| | | TGGTTCTGGCCTTATGAATACAACTATTGGGGACAGGGAACTCAGGTGACGGTC |
| | | TCTTCCGGTGGCTCTGGGGGCAGCGGTGGGAGGGGCCAAGTGCAGCTTCAGGAG |
| | | TCCGGCGGAGGCAGCGTTCAAGCAGGGGGTAGCCTGAGACTCTCATGTGTAGCC |
| | | TCCGCTTCCACTTACTGCACCTACGACATGCACTGGTATCGTCAGGCTCCTGGC |
| | | AAGGGCCGCGAGTTTGTGTCCGCCATCGACTCCGACGGCACAACGCGGTACGCT |
| | | GACAGCGTGAAGGGCCGCTTTACCATTAGCCAAGGCACCGCCAAGAATACAGTG |
| | | TACCTGCAAATGAATAGCCCGCAACCGGAGGATACCGCGATGTACTATTGCAAG |
| | | ACGGTCTGTGTTGTGGGCAGTCGGGGAGCGACTACTGGCGGGCAAGGTACACAA |
| | | GTCACTGTGTCCTCC |
| hIL27 | 1347 | CAGGTGCAGGTCCAGGAATCTGGAGGTGGCTCTGTGCAGAGTGGGGGCAGCCTT |
| Ra_VH | | CGCCTGTCCTGCGCTGCCAGCGGCTTCACATACAGTACGTCAAACTCCTGGATG |
| H9- | | GCCTGGTTCCGTCAAGCGCCTGGAAAGGAACGCGAGGGAGTGGCTGCGATTTAT |
| DR595 | | ACCGTCGGGGGGTCTATTTTTTACGCCGACTCCGTCAGAGGACGTTTCACGATC |
| | | TCCCAGGACGCCACGAAGAATATGTTTTATCTCCAGATGAACACACTTAAACCC |
| | | GAAGACACCGCAATGTATTACTGCGCTGCCGCATCTGGCCGCCTCCGGGGCAAA |
| | | TGGTTTTGGCCTTACGAGTACAATTACTGGGGCCAGGGAACACAGGTTACCGTG |
| | | AGCAGTGGAGGCGGGTCCCAGGTTCAGTTGCAGGAGTCAGGGGGGGGAAGCGTG |
| | | CAGGCCGGAGGTTCACTGACCCTGTCTTGTGCAGCGTCTGAATACGCCTACAGC |
| | | ACCTGTAACATGGGAATGGTATCGCCAGGCTCCTGGAAAGGAAAGGAGCTGGTG |
| | | TCTGCCTTTATCTCTGACGGCAGCACTTATTACGCTGACTCCGTGAAGGGACGC |
| | | TTTACCATCACCCGCGACAACGCGAAGAACACTGTGTATCTTCAGATGAACTCC |
| | | CTGAAGCCCGAAGACACCGCGATCTACTATTGGTCCGCTAATTGTTACCGTCGC |
| | | CTCCGCAACTATTGGGGCCAAGGGACCCAGGTGACCGTTAGTTCG |
| hIL27 | 1348 | CAAGTCCAGGTTCAGGAGTCCGGCGGTGGCAGCGTGCAGTCTGGAGGGTCCCTG |
| Ra_VH | | CGCTTGTCCTGCGCCGCTTCCGGTTTCACGTATTCCACAAGTAATAGTTGGATG |
| H9- | | GCTTGGTTTAGACAGGCCCCCGGCAAAGAGCGCGAGGGTGTGGCTGCCATCTAT |
| DR595 | | ACCGTCGGTGGGAGCATCTTTTACGCCGATTCCGTGCGTGGCCGCTTCACCATC |
| | | AGCCAGGATGCCACAAAGAATATGTTTTAGCTGCAGATGAACACCCTGAAGCCC |
| | | GAAGACACCGCTATGTACTATTGCGCGGCAGCTTCTGGACGGCTGCGCGGTAAG |
| | | TGGTTTTGGCCATACGAGTACAACTATTGGGGCCAGGGCACTCAGGTGACCGTG |
| | | TCATCCGGGGGCTCCGGCGGGAGCGGTGGCTCTGGCCAGGTGCAGCTCCAGGAG |
| | | TCCGGGGGTGGCTCTGTCCAGGCTGGAGGCAGTCTGACCCTGTCCTGTGCCGCG |
| | | TCTGAGTACGCCTACTCCACTTGTAATATGGGCTGGTATCGTCAGGCACCTGGC |
| | | AAAGAACGCGAACTGGTGTCCGCATTTATTTCCGACGGTAGTACCTATTACGCT |
| | | GATTCCGTGAAGGGACGCTTCACCATCACACGGGATAACGCCAAGAACACTGTG |
| | | TATCTTCAGATGAACTCCTTGAAGCCCGAAGACACCGCTATCTACTATTGCAGC |
| | | GCCAACTGCTACAGACGGCTGCGTAACTACTGGGGCCAGGGCACTCAGGTGACT |
| | | GTCTCCTCT |
| hIL27 | 1349 | CAGGTGCAGCTTCAGGAATCCGGGGGGGGCAGCGTCCAGAGCGGCGGTTCCCTC |
| Ra_VH | | CGCCTCAGCTGTGCGGCTTCTGGATTCACCTATTCTACAAGCAATTCATGGATG |
| H9- | | GCGTGGTTTCGCCAAGCGCCGGGCAAAGAGCGTGAGGGCGTTGCGGCTATTTAC |
| DR596 | | ACTGTCGGAGGCTCCATCTTCTACGCAGACTCAGTGCGTGGCCGTTTCACTATC |
| | | TCCCAGGACGCCACTAAGAATATGTTCTATCTCCAGATGAATACCCTCAAACCA |
| | | GAAGACACCGCAATGTATTACTGCGCTGCCGCATCTGGCCGTCTGAGAGGTAAG |
| | | TGGTTTTGGCCTTACGAGTACAACTACTGGGGACAAGGCACTCAGGTCACAGTC |
| | | TCCTCAGGCGGTGGCTCCCAGGTGCAGGTCCAAGAGTCAGGCGGGGGTCTGGTC |
| | | CAGCCTGGGGGGTCCCTTAGGCTTAGCTGTACCGCGTCCGGTCTCACCTTCGAT |
| | | GACAGCGTCATGGGATGGTTCCGCCAGGCCCCAGGCAAGGGGAGGGAGGCAGTG |
| | | AGCTGCATCTCCAGCTCCGGCGCTAACGCCTTTTATGCGGACAGTGTCAAAGGC |
| | | AGGTTCACGATTTCTAGGGATAACGCTAAGAACACCCTGTACCTCCAGATGAAC |
| | | AGCCTCAAACCGGAGGATACCGCGACTTACTATTGCAAACGTGGTCACGCCTGC |
| | | GCTGGTTATTACCCCATCCCGTATGATGACTATTGGGGCCAAGGCACCCAGGTG |
| | | ACTGTTTCTTCC |
| hIL27 | 1350 | CAGGTCCAGTTGCAGGAGTCAGGCGGGGGCTCTGTGCAGTCTGGAGGCAGTCTC |
| Ra_VH | | AGACTGTCTTGCGCGGCTTCCGGCTTCACATACTCCACTTCCAACAGTTGGATG |
| H9- | | GCCTGGTTCAGGCAAGCGCCGGGCAAGGAGAGAGAGGGTGTAGCGGCAATCTAT |
| DR596 | | ACTGTCGGCGGTTCAATTTTTTACGCGGACTCTGTTCGTGGCAGATTCACCATT |
| | | TCCCAGGACGCCACAAAAAATATGTTCTACCTCCAAATGAACACCCTCAAGCCT |
| | | GAGGACACTGCTATGTATTACTGTGCGGCTGCCTCAGGCCGTCTGCGGGGTAAG |
| | | TGGTTTTGGCCCTACGAGTACAACTACTGGGGCCAGGGGACGCAGGTGACAGTC |
| | | AGCTCAGGGGGCAGCGGCGGGTCCGGCGGGAGTGGCCAGGTGCAGCTCCAGGAA |
| | | TCTGGCGGAGGTCTTGTCCAGCCTGGCGGGTCTTTGCGTCTGTCGTGTACCGCA |
| | | AGTGGTCTGACCTTCGATGACTCAGTTATGGGATGGTTCAGACAAGCGCCTGGT |
| | | AAGGGCCGCGAGGCGGTGTCATGTATCTCTTCCAGCGGAGCCAACGCTTTTTAC |
| | | GCCGACTCCGTGAAAGGCAGGTTTACGATTAGTCGGGATAATGCCAAGAACACC |
| | | TTGTACCTCCAGATGAACAGCTTGAAGCCCGAAGACACTGCTACCTATTACTGT |
| | | AAGCGCGGTCACGCCTGCGCGGGCTATTACCCAATCCCCTACGATGACTACTGG |
| | | GGCCAGGGAACCCAGGTGACAGTTTCCAGC |
| hIL27 | 1351 | CAGGTGCAGCTGCAAGAATCCGGCGGTGGCTCCGTTCAGGCCGGAGGCTCATTG |
| Ra_VH | | CGTCTCTCCTGTCGTGCGTCTGGGTCCACCTACTCTAACTATTGCCTGGGCGGG |
| H10- | | TTCCGGCAGATTACGGGTAAGGAACGCGAAGGAGTTGCCGTGATTAACTGGGTC |
| DR591 | | GGTGGAATGGTGTACTTTGCTGACTCCGTCAAGGGTCGGTTCACCGTCTCCCAG |
| | | GACCAGGCTAAGAACACCCTGTACTTGCAGATGAACTCCCTCAAGCCTGAGGAC |
| | | ACCGCAATGTATTACTGTGCCGCTGAGTCAGTCAGCTCCTTCTCCTGGGGGGGG |
| | | TGGCTCACTCGCCCCGACAGAGTCCCCTATTGGGGGCAGGGAACCCAGGTGACC |
| | | GTCTCTAGCGGTGGGGGCTCCCAGGTGCAGTTGCAGGAATCTGGTGGGGGCTCC |
| | | GTCCAAGCCGGTGGCTCACTTAGGCTGTCCTGTACGGCAAGGGGGGCCATCGCC |
| | | TCTGGTTATATCGACTCACGGTGGTGTATGGCATGGTTCAGACAGGCTCCAGGG |
| | | AAGGAGCGGGAGGGAGTCGCTGCCATCTGGCCGGGGGGGGGCCTCACCGTTTAC |
| | | GCAGATAGCGTGAAGGGTAGGTTTACCATCTCTCGCGACCACGCCAAAAATACT |
| | | CTGTACCTCCAAATGAATAACCTGAAGCCCGAAGATACCGCCATGTACTATTGC |
| | | GCCGCTGGCTCCCCGCGCATGTGCCCTTCTCTGGAGTTCGGCTTCGATTATTGG |
| | | GGACAAGGAACCCAGGTGACAGTGTCTTCC |
| hIL27 | 1352 | CAGGTACAGGTTCAGGAAAGCGGGGGAGGCTCAGTGCAGGCTGGCGGAAGCCTT |
| Ra_VH | | CGCCTGTCATGTCGGGCCTCTGGTTCCACCTACAGTAACTACTGCCTGGGCTGG |
| H10- | | TTCCGCCAGATCACTGGCAAGGAACGCGAGGGCGTCGCGGTCATCAACTGGGTC |
| DR591 | | GGCGGAATGTTGTACTTCGCAGATAGCGTCAAGGGCAGGTTCACAGTCTCCCAG |
| | | GATCAGGCCAAGAACACCCCGTATCTGCAAATGAACTCCCTCAAGCCTGAAGAT |
| | | ACCGCCATGTATTACTGCGCAGCCGAGTCTGTCTCTTCATTCCCTTGCGGGGGT |
| | | TGGCTGACCCGTCCTGACAGGGTGCCATACTGGGGCCAGGGTACACAAGTCACC |
| | | GTGTCTTCAGGAGGTTCCGGTGGGTCAGGAGGTTCCGGGCAAGTTCAGCTCCAA |
| | | GAGAGTGGTGGCGGTAGCGTGCAGGCAGGCGGTTCACTTCGTCTGTCTTGCACA |
| | | GCCAGTGGGGCTATCGCCTCCGGCTACATTGACAGCAGATGGTGTATGGCCTGG |
| | | TTCCGGCAGGCCCCAGGCAAAGAAAGAGAAGGGCGGGCAGCCATCTGGCCGGGT |
| | | GGGGGCCTGACGGTCTATGCTGACAGCGTGAAGGGCCGCTTCACTATCTCAAGG |
| | | GATCACGCCAAGAACACCTTGTACTTGCAGATGAACAATCTGAAGCCCGAAGAC |
| | | ACCGCAATGTATTACTGTGCCGCAGGTAGCCCCCGCATGTGCCCATCCCTGGAG |
| | | TTTGGATTCGACTACTGGGGCCAGGGAACCCAGGTGACCGTTAGCTCT |
| hIL27 | 1353 | CAGGTTCAGCTTCAGGAGAGCGGCCGGGGGTCCGTGCAGGCCGGTGGCTCCCTG |
| Ra_VH | | CGCCTGAGCTGCCGCGCCAGCGGAAGCACTTACAGCAATTATTGTCTTGGGTGG |
| H10- | | TTCCGTCAGATCACAGGCAAAGAGCGCGAAGGCGTGGCCGTTATCAACTGGGTC |
| DR592 | | GGCGGTATGCTGTATTTCGCCGACTCTGTGAAAGGTCGCTTTACGGTCTCTCAG |
| | | GATCAGGCCAAGAACACGCTTTACTTGCAAATGAACTCACTGAAGCCCGAAGAC |
| | | ACCGCCATGTATTACTGTGCCGCAGAGTCTGTCAGCCCCTTTTCATGCGGAGGT |
| | | TGGTTGACTCGCCCCGACCGTGTTCCTTACTGGGGCCAGGGCACGCAAGTGACC |
| | | GTTTCCTCTGGAGGCGGAAGTCAGGTGCAGTTGCAGGAGTCCGGGGGGGGTAGC |
| | | GTGCAGGCCGGTGGCAGTCTGCGTCTGAGTTGTACTGCCCCCGGCTTCACCTCC |
| | | AACTCCTGTGGCATGGACTGGTATCGCCAGGCCCCCGGAAAAGAACGTGAGTTC |
| | | GTATCTAGCATCTCCACCGATGGGACCACAGGTTATGCTGATTCGGTAAAGGGT |
| | | AGGTTCACTATCAGCAAGGATAAAGCCAAAGATACCGTGTACCTCCAGATGAAT |
| | | AGCCTTAAACCAGAGGATACGGGAATGTATAGTTGCAAGACCAAGGATGGAACT |
| | | ATCGCGACAATGGAACTCTGCGACTTCGGCTACTGGGGCCAGGGAACTCAGGTG |
| | | ACCGTGTCCTCC |
| hIL27 | 1354 | CAGGTTCAGTTGCAGGAATCCGGGGGGGGAAGCGTGCAAGCAGGGGGTAGTTTG |
| Ra_VH | | CGCCTGTCATGCCGCGCCAGGGGCTCCACCTACAGCAATTATTGTCTGGGTTGG |
| H10- | | TTCCGCCAGATCACTGGCAAGGAGCGCGAAGGGGTGGCCGTGATTAACTGGGTG |
| DR592 | | GGCGGTATGTTGTACTTCGCTGACTCCGTAAAGGGACGCTTCACTGTCAGCCAG |
| | | GACCAGGCTAAGAACACTCTGTATCTCCAGATGAACTCACTGAAGCCTGAGGAT |
| | | ACCGCCATGTATTACTGCGCTGCCGAGTCCGTCTCCAGGTTTTCCTGGGGCGGA |
| | | TGGCTGACTCGCCCTGACCGCGTGCCGTACTGGGGGCAAGGCACTCAGGTGACA |
| | | GTGTCTAGCGGAGGGTCTGGGGGCTCTGGTGGAAGCGGGCAGGTGCAGCTTCAG |
| | | GAATCTGGCGGTGGGAGCGTACAAGCGGGGGGATCTCTTCGTCTGTCCTGTACC |
| | | GCTCCTGGCTTTACGTCCAATTCCTGTGGGATGGATTGGTACAGACAAGCCCCA |
| | | GGTAAGGAGCGCGAGTTCGTGTCCTCTATCAGCACCGACGGAACTACGGGGTAC |
| | | GCTGACTCAGTGAAGGGTCGCTTCACCATCAGCAAGGATAAGGCCAAGGATACC |
| | | GTGTACCTGCAAATGAACAGTCTGAAGCCAGAAGATACCGGCATGTACTCATGC |
| | | AAGACGAAAGATGGGACTATCGCCACTATGGAACTGTGCGATTTTGGCTACTGG |
| | | GGGCAGGGCACTCAGGTGACTGTCTCAAGC |
| hIL27 | 1355 | CAGGTCCAGCTCCAGGAATCTGGTGGAGGTAGCGTTCAGGCCGGGGGCTCCCTC |
| Ra_VH | | CGCCTGTCATGCAGGGCCAGCGGCTCTACCTATTCCAATTACTGCCTCGCGGGG |
| H10- | | TTCAGACAGATCACAGGAAAGGAGCGCGAGGGCGTGGCAGTCATCAACTGGGTA |
| DR593 | | GGCGGTATGCTGTATTTCGCGGATAGTGTGAAGGGAAGATTCACCGTGAGTCAG |
| | | GATCAGGCTAAGAACACCCTGTATCCCCAGATGAACTCCTTGAAGCCCGAGGAC |
| | | ACTGCCATGTATTACTGCGCAGCCGAATCTGTTAGCTCGTTCAGCTGTGGTGGC |
| | | TGGCTGACCCGCCCAGATCGCGTGCCATATTGGGGCCAGGGCACACAGGTTACT |
| | | GTCTCCAGTGGCGGGGGCAGCCAAGTGCAGTTGCAGGAGAGCGGAGGCGGAAGT |
| | | GTGCAGGCAGGCGGATCTCTGAGACTCTCTTGTGCTGCCTCCGGGTATCCATAT |
| | | TCTAATGGCTACATGGGATGGTTCCGCCAAGCACCGGGCAAAGAGAGAGAAGGG |
| | | GTTGCTACCATCTATACCGGCGATGGCAGAACCTATTACGCGGACTCCGTGAAG |
| | | GGACGCTTCACCATCAGCCGTGATAACGCCAAGAACACTGTGGACCTCCAGATG |
| | | TCTTCCCTGAAGCCTGAAGACACCGCTATGTACTATTGTGCTGCACGCGCCGGG |
| | | CCACTTTACAGTAGCGGTTCTCCTCTTACCCGCGCTCGCTACAACGTCTGGGGG |
| | | CAGGGTACTCAGGTGACAGTTAGTAGC |
| hIL27 | 1356 | CAAGTGCAGTTGCAGGAAAGCGGAGGCGGGAGCGTACAGGGGGGGGGTTCTCTG |
| Ra_VH | | CGGCTGAGCTGCCGCGCATCTGGTTCCACATACAGCAATTATTGTCTGGGTTGG |
| H10- | | TTTAGGCAGATCACTGGTAAGGAGAGGGAAGGGGTCGCAGTTATAAATTGGGTG |
| DR593 | | GGTGGGATGCTCTACTTCGCCGATTCAGTGAAGGGCCGGTTCACCGTGAGTCAG |
| | | GATCAGGCGAAGAATACTCTGTACCTCCAGATGAATAGCCTGAAGCCCGAGGAC |
| | | ACCGCCATGTATTACTGCGCCGCAGAGAGTGTCAGCTCCTTTAGCTGCGGGGGC |
| | | TGGCTGACCGGCCCGGACCGCGTGCCCTACTGGGGCCAGGGGACCCAGGTTACC |
| | | GTTAGCTCTGGCGGTAGCGGGGGCTCTGGAGGCAGCGGACAAGTCCAGCTGCAA |
| | | GAATCCGGGGGAGGCAGCGTACAGGCTGGCGGGTCTCTGCGCCTGTCCTGCGCC |
| | | GCTTCAGGATACCCGTATAGCAACGGCTATATGGGTTGGTTCAGACAGGCCCCC |
| | | GGCAAGGAACGTGAAGGAGTGGCCACCATCTACACCGGCGACGGGCGCACCTAT |
| | | TACGCTGATTCTGTGAAGGGCCGCTTTACAATCAGCCGCGATAACGCTAAGAAC |
| | | ACCGTTGACGTTCAAATGTCCAGCCTGAAGCCGGAGGATACCGCCATGTATTAC |
| | | TGTGCAGCCCGCGCCGCACCACTCTACAGCTCCGGTTCTCCCCTCACAAGGGCT |
| | | CGGTACAATGTTTGGGGCCAGGGCACCCAGGTCACCGTCTCTAGC |
| hIL27 | 1357 | CAGGTGCAGCTCCAGGAATCAGGTGGGGGCAGCGTACAGGCAGGGGGTTCACTC |
| Ra_VH | | CGCCTGTCTTGCCGGGCCTCTGGCAGTACATACTCCAATTATTGCCTGGGTTGG |
| H10- | | TTTCGCCAAATTACCGGCAAGGAGCGGGAGGGCGTCGCTGTAATCAACTGGGTG |
| DR594 | | GGCGGGATGCTCTATTTTGCTGACTCCGTTAAGGGTCGTTTCACGGTCAGCCAG |
| | | GACCAGGCCAAAAATACACTGTATCTCCAGATGAACTCCCTCAAACCCGAAGAC |
| | | ACCGCCATGTATTACTGTGCTGCCGAGAGTGTCAGCTCTTTTTCCTGGGGGGGG |
| | | TGGCTTACCCGCCCGGACCGTGTCCCATACTGGGGTCAAGGCACCCAGGTTACC |
| | | GTCTCATCCGGCGGAGGCAGCCAGGTGCAACTCCAGGAGAGCGGGGGGGGTAGC |
| | | GTGCAGGCAGGCGGAAGCCTGCGTCTCTCCTGTGTGGCTTCCGCGTCCACCTAC |
| | | TGTACCTATGATATGCACTGGTATCGCCAGGCTCCTGGAAAGGGCCGCGAGTTC |
| | | GTGAGTGCTATTGATTCCGATGGCACCACTCGGTACGCTGACTCCGTGAAGGGA |
| | | CGTTTCACCATCTCCCAGGGTACAGCTAAGAACACCGTGTACCTCCAGATGAAC |
| | | TCCCTCCAGCCCGAGGATACCGCAATGTATTACTGCAAGACCGTTTGTGTAGTG |
| | | GGCTCACGCTGGTCCGACTATTGGGGCCAGGGGACCCAGGTGACTGTATCATCT |
| hIL27 | 1358 | CAGGTCCAGCTTCAGGAAAGCGGTGGGGGCTCCGTGCAGGCCGGAGGCTCCCTG |
| Ra_VH | | CGTCTGAGCTGTCGGGCCTCTGGTTCCACCTACTCCAACTACTGTCTGGGCTGG |
| H10- | | TTCCGCCAGATTACAGGCAAGGAACGCGAGGGTGTGGCGGTCATAAATTGGGTG |
| DR594 | | GGTGGAATGCTGTATTTCGCGGATAGCGTGAAAGGGCGCTTCACCGTGTCTCAG |
| | | GACCAGGCTAAGAACACACTGTACTTGCAGATGAACAGCCTGAAGCCCGAAGAC |
| | | ACTGCCATGTACTATTGTGCCGCTGAGTCTGTGTCCTCTTTCACGTGGGGCGGA |
| | | TGGCTGACCCGCCCCGACAGGGTGCCTTACTGGGGTCAGGGCACCCAGGTGACG |
| | | GTTTCTAGTGGTGGCTCAGGTGGCTCCGGCGGTTCCGGCCAGGTCCAGTTGCAG |
| | | GAGTCTGGAGGTGGCAGCGTGCAGGCCGGTGGCAGCCTGCGCCTGTCATGTGTC |
| | | GCTTCTGCGAGCACCTATTGTACCTATGATATGCACTGGTATCGCCAGGCCCCA |
| | | GGCAAGGGTAGGGAGTTTGTGTCCGCGATTGACTCTGACGGCACCACGCGCTAT |
| | | GCGGATTCCGTGAAGGGGCGTTTTACAATCTCCCAGGGCACCGCTAAGAACACT |
| | | GTTTATCTTCAGATGAACAGCTTGCAGCCTGAGGACACCGCGATGTATTACTGT |
| | | AAAACTGTCTGTGTAGTGGGCTCCAGGTGGTCTGACTACTGGGGTCAGGGCACC |
| | | CAGGTGACGGTTTCCAGC |
| hIL27 | 1359 | CAGGTGCAGTTGCAGGAATCTGGGCGGGGCTCTGTGCAGGCGGGCGGGTCCCTG |
| Ra_VH | | CGCTTGTCCTGCCGCGCTTCTGGTTCTACCTACTCCAACTATTGTCTGGGCTGG |
| H10- | | TTTAGGCAGATCACCGGAAAGGAGCGCGAAGGTGTGGCTGTTATACAATTGGGG |
| DR595 | | GGCGGGATGGTCTATTTTGCAGACTCCGTCAAGGGCCGCTTCACCGTCTCACAG |
| | | GATCAAGCCAAGAATACGCTGTATCTTCAGATGAACAGCCTGAAGCCCGAGGAT |
| | | ACAGCCATGTATTACTGCGCCGCAGAGTCTGTGTCTTCCTTTTCTTGCGGAGGC |
| | | TGGCTCACACGCCCGGACCGCGTGCCCTACTGGGGCCAGGGCACCCAGGTGACC |
| | | GTATCCAGCGGGGGGGGTTCCCAAGTGCAGCTCCAGGAGTCTGGCGGAGGGTCC |
| | | GTGCAGGCTGGTGGGAGTCTGACCTTGAGTTGCGCAGCCAGCGAATACGGTTAC |
| | | TCCACTTGCAACATGGGTTGGTATCGTCAGGGTCCCGGTAAAGAGCGCGAGCTG |
| | | GTCTCTGCATTCATCTCAGATGGCTCTACTTACTATGCTGACTCCGTGAAGGGT |
| | | CGGTTTACTATCACCCGCGACAACGCCAAGAACACAGTGTACTTGCAGATGAAC |
| | | TCCCTGAAGCCCGAGGATACCGCCATTTACTATTGTTCCGCCAACTGTTACAGA |
| | | CGCCTGCGCAACTATTGGGGGCAGGGAACACAGGTTACTGTGTCCTCT |
| hIL27 | 1360 | CAGGTGCAGTTGCAGGAGAGCGGAGGTGGCAGCGTCCAGGCCGGAGGTTCTCTG |
| Ra_VH | | CGTCTGAGCTGCCGTGCATCCGGTAGTACATACTCTAACTACCGTCTCGGCTGG |
| H10- | | TTCAGGCAGATCACAGGCAAGGAAAGGGAGGGTGTTGCCGTAATCAACTGGGTT |
| DR595 | | GGCGGTATGCTCTACTTCGCTGACTCCGTGAAGGGTAGATTTACCGTGTCTCAG |
| | | GACCAAGCTAAGAACACCCTGTACTTGCAGATGAACAGCCTGAAACCGGAAGAT |
| | | ACCGCCATGTACTATTGCGCTGCCGAGTCCGTGTCCTGTTTCAGCTGGGGGGGT |
| | | TGGCTGACCAGACCTGATCGCGTTCCGTACTGGGGTCAAGGCACACAGGTTACC |
| | | GTGTCAAGCGGTGGCTCTGGAGGCTCCGGTGGGTCCGGTCAGGTCCAGTTGCAA |
| | | GAGTCTGGAGGTGGCAGCGTCCAGGCCGGAGGGTCCCTCACCCTGAGTTGTGCG |
| | | GCCAGTGAGTACGCCTACTCCACTTGCAACATGGGATGGTATCGCCAGGCCCCA |
| | | GGAAAGGAGCGCGAGCTGGTCTCCGCTTTCATCTCCGACGGATCTACCTATTAC |
| | | GCCGACTCTGTGAAGGGACGTTTTACTATCACTAGGGATAACGCGAAAAACACT |
| | | GTGTACCTCCAGATGAACTCTCTCAAGCCTGAGGACACGGCCATCTACTATTGG |
| | | TCTGCGAACTGTTACAGACGGGTGAGAAATTATTGGGGACAGGGTACTCAGGTG |
| | | ACCGTTAGCAGC |
| hIL27 | 1361 | CAGGTTCAGGTCCAGGAATCTGGGGGAGGCTCTGTCCAAGCTGGGGGTTCCTTG |
| Ra_VH | | CGTCTGTCTTGTAGGGCCTCTGGGAGCACCTACTCCAATTACTGTCTGCGGGGG |
| H10- | | TTTCGGCAGATCACCGGCAAAGAACGCGAGGGCGTGGCCGTCATCAACTGGGTG |
| DR596 | | GGCGGAATGCTGTACTTTGCCGACTCTGTGAAGGGCCGGTTCACGGTGTCACAG |
| | | GACCAGGCCAAGAATACCCTGTATCTTCAGATGAACTCTCTCAAGCCCGAGGAC |
| | | ACTGCTATGTATTACTGTGCTGCGGAGAGCGTCTCATCCTTCACGTGTGGTGGC |
| | | TGGCTGACACGCCCGGACCGTGTCCCTTATTGGGGTCAGGGAACCCAGGTGACC |
| | | GTTAGCTCAGGAGGTGGATCTCAGGTGCAACTCCAGGAGAGCGGCGGTGGCCTG |
| | | GTGCAACCCGGAGGTAGCTTGCGCTTGTCCTGTACTGCCTCTGGACTCACCTTC |
| | | GATGACTCAGTAATGGGTTGGTTCAGACAGGCCCCCGGAAAGGGCAGGGAGGCA |
| | | GTGTCTTGTATTAGCTCCAGGGGGGCTAATGCGTTCTACGCCGATTCCGTCAAG |
| | | GGCAGGTTTACCATTAGTCGCGATAACGCCAAGAACACCCTTTACTTGCAGATG |
| | | AACAGCCTGAAGCCCGAGGACACAGCCACCTATTACTGTAAGAGAGGTCACGCC |
| | | TGTGCTGGCTATTACCCAATTCCTTACGATGACTACTGGGGACAGGGAACCCAA |
| | | GTCACTGTCTCTTCT |
| hIL27 | 1362 | CAGGTCCAGCTTCAGGAGTCCGGGGGGGGTTCCGTCCAGGCCGGGGGATCACTT |
| Ra_VH | | CGCCTGTCTTGTCGCGCCTCAGGCTCTACTTACTCCAATTATTGCCTCGGTTGG |
| H10- | | TTCCGCCAAATCACCGGCAAAGAACGGGAGGGAGTAGCCGTAATTAACTGGGTT |
| DR596 | | GGGGGAATGCTGTATTTCGCCGACAGTGTGAAGGGCCGCTTCACCGTGTGTCAG |
| | | GACCAGGCAAAGAATACCCTGTATCTCCAGATGAACTCCCTGAAGCCGGAGGAC |
| | | ACTGCCATGTATTACTGCGCCGCTGAGTCCGTGTCCAGCTTCTCCTGCGGTGGC |
| | | TGGCTGACCCGCCCAGATCGTGTTCCTTATTGGGGCCAGGGCACTCAGGTCACA |
| | | GTCAGCTCTGGAGGCTCAGGAGGTTCTGGCGGTAGTGGTCAGGTTCAGCTCCAG |
| | | GAAAGCGGAGGCGGATTGGTTCAGCCTGGTGGAAGTCTGCGCCTCTCTTGCACC |
| | | GCCTCCGGCCTGACATTTGACGATAGCGTGATGGGCTGGTTCCGCCAGGGTCCG |
| | | GGAAAGGGTAGAGAGGCTGTGTCTTGTATCAGCTCTTCGGGGGCGAACGCCTTC |
| | | TACGCCGACTCCGTGAAGGGCCGTTTCACCATCTCACGCGACAACGCAAAGAAC |
| | | ACTCTGTATCTCCAGATGAACAGTCTGAAGCCCGAAGACACGGCGACCTACTAT |
| | | TGCAAGCGCGGCCACGCCTGCGCCGGGTACTATCCCATCCCATACGATGACTAT |
| | | TGGGGCCAGGGAACCCAAGTGACCGTATGTTCA |
| hIL27 | 1363 | CAGGTGCAGGTTCAGGAGAGTGGTGGAGGCTCCGTGCAGGCCGGTGGCAGCCTC |
| Ra_VH | | CGTCTCTCTTGCAGAGCCTCAGGAAGCACCTACAGCAATTACTGTTTGGGCGGG |
| H11- | | TTTCGGCAGTCAACGGGCAAGGAGAGAGAGGGCGTGGCCGTTATCAACTGGGTG |
| DR591 | | GGCGGTATGCTGTACTTCGCCGACTCTGTGAAGGGTCGGTTCACAGTGAGCCAG |
| | | GACCACGCGAAGAATACAGTGACTCTCCAGATGAACTCCCTCAAGCCCGAGGAC |
| | | ACCGCCATGTACTATTGTGCGGCTGAGTCCGTATCCTCTTTTTCGTGCGGGGGC |
| | | TGGCTGACTAGGCCTGGCCGTGTCCCATACTGGGGGCAGGGCACTCAGGTCACA |
| | | GTCAGCAGTGGAGGCGGTAGTCAGGTCCAGTTGCAGGAAAGCGGGGGAGGTTCC |
| | | GTCCAGGCCGGAGGTTCCCTGCGCCTGAGCTGCACCGGTTCGGGCGCAATCGCC |
| | | TCTGGTTACATCGACTCCCGCTGGTGTATGGCCTGGTTCCGCCAGGCCCCTGGA |
| | | AAGGAGCGCGAGGGTGTCGCTGCGATCTGGCCTGGTGGAGGCCTGACTGTCTAT |
| | | GCTGATTCCGTAAAGGGCCGTTTCACTATCTCTCGCGATCATGCCAAAAACACC |
| | | CTGTACTTGCAGATGAATAACCTGAAGCCCGAAGACACTGCCATGTATTACTGT |
| | | GCCGCAGGCTCTCCTAGAATGTGCCCTTCCCTGGAGTTCGGGTTTGATTATTGG |
| | | GGGCAGGGCACCCAGGTTACTGTGTCTTCC |
| hIL27 | 1364 | CAAGTCCAGCTCCAGGAGAGCGGGGGAGGCTCCGTGCAGGCCGGTGGCAGCCTC |
| Ra_VH | | CGCCTCAGCTGCCGTGCTTCAGGAAGCACCTACAGCAACTACTGCTTGGGTTGG |
| H11- | | TTTCGTCAGAGCACCGGAAAAGAGCGCGAGGGTGTGGCTGTGATTAACTGGGTC |
| DR591 | | GGAGGGATGGTGTATTTTGCGGATAGCGTGAAGGGGCGCTTCACAGTGTCTCAG |
| | | GATCATGCCAAGAACACAGTGACCCTTCAGATGAACAGCCTCAAGCCTGAGGAC |
| | | ACCGCCATGTATTACTGCGCTGCCGAGTCCGTCAGCTCCTTCAGCTGGGGGGGC |
| | | TGGCTCACCCGCCCTGGCCGCGTGCCCTATTGGGGCCAGGGAACCCAGGTTACT |
| | | GTTTCCAGTGGAGGGTCCGGGGGCTCTGGAGGCAGCGGCCAGGTACAGTTGCAG |
| | | GAATCCGGGGGGGGCTCAGTCCAGGCGGGAGGTTCCTTGCGCCTGTCCTGTACC |
| | | GCGTCCGGTGCAATCGCCTCCGGCTATATTGATTCCAGATGGTGTATGGCATGG |
| | | TTCCGCCAGGCTCCCGGTAAGGAACGGGAGGGGGTTGCGGCCATCTGGCCAGGA |
| | | GGGGGTCTCACCGTGTATGCCGACAGTGTTAAGGGCCGTTTCACTATCAGCAGG |
| | | GACCATGCAAAGAACACGCTGTACCTCCAAATGAACAATCTGAAGCCGGAAGAC |
| | | ACCGCCATGTATTACTGTGCTGCCGGTAGTCCTAGAATGTGTCCATCTCTGGAG |
| | | TTCGGTTTTGATTACTGGGGTCAGGGCACACAAGTTACCGTGTCTAGC |
| hIL27 | 1365 | CAGGTACAACTCCAGGAGTCTGGTGGGGGCAGCGTACAGGCCGGAGGCTCTCTC |
| Ra_VH | | AGACTCTCATGTCGCGCGAGCGGCTCCACCTATAGCAACTATTGGCTGGGGTGG |
| H11- | | TTCAGACAGAGCACTGGTAAGGAGCGTGAAGGTGTGGCAGTTATCAACTGGGTC |
| DR592 | | GGGGGAATGCTGTATTTCGCCGACAGCGTGAAGGGCCGGTTCACTGTGTCCCAG |
| | | GATCATGCCAAAAACACCGTCACCCTCCAGATGAACTCCCTGAAGCCCGAGGAC |
| | | ACAGCTATGTATTACTGTGCTGCGGAGAGCGTTAGCTCTTTCAGCTGTGGTGGC |
| | | TGGCTCACCCGGCCAGGCCGTGTCCCATATTGGGGACAGGGCACCCAGGTGACA |
| | | GTGTCTAGCGGCGGTGGCTCCCAGGTCCAGCTTCAGGAGTCCGGCGGTGGATCT |
| | | GTGCAGGCAGGCGGTTCCCTCAGGCTGAGTTGTACCGCCCCCGGCTTCACCAGC |
| | | AACAGCTGCGGTATGGACTGGTATCGCCAGGGTCCCGGAAAGGAGAGAGAGTTC |
| | | GTGAGTTCTATCTCCACTGATGGGACGACCGGCTACGCCGACTCCGTGAAGGGG |
| | | CGCTTCACTATCTCTAAAGATAAGGCTAAGGATACCGTCTACTTGCAGATGAAT |
| | | AGCTTGAAACCTGAAGATACCGGCATGTACTCCTGCAAGACCAAGGATGGGACC |
| | | ATTGCTACGATGGAACTCTGTGACTTCGGCTACTGGGGACAGGGCACTCAGGTG |
| | | ACCGTCTCCAGT |
| hIL27 | 1366 | CAGGTGCAGCTTCAGGAGTCTGGAGGCGGATCTGTGCAGGCAGGCGGTAGTCTG |
| Ra_VH | | CGTCTTAGCTGCCGTGCCTCTGGCTCAACCTATTCCAATTATTGCCTCGGCTGG |
| H11- | | TTTCGGCAGTCCACCGGCAAGGAAAGGGAAGGCGTGGGGGTCATCAACTGGGTT |
| DR592 | | GGGGGTATGCTGTATTTCGCCGACAGCGTGAAGGGCCGTTTACAGTTGTCTCAA |
| | | GACCATGCTAAGAATACCGTTACCCTCCAGATGAACTCTCTCAAACCGGAAGAC |
| | | ACTGCTATGTATTACTGCGCAGCCGAATCCGTTTCTAGCTTCAGCTGCGGGGGT |
| | | TGGCTGACTAGGCCTGGGGGGGGCCCTATTGGGGGCAGGGTACACCAGGTAACC |
| | | GTTTCCAGCGGAGGCTCTGGGGGGAGTGGCGGATCTGGCCAGGTGCAGTTGCAG |
| | | GAATCTGGAGGCGGATCTGTCCAAGCTGGGGGCAGCCTCCGTCTGTCATGTACC |
| | | GCCCCTGGGTTCACTTCCAACTCCTGCGGCATGGATTGGTATCGCCAGGCACCT |
| | | GGGAAGGAGCGCGAATTTGTAAGCTCAATCTCCACAGATGGCACCACTGGCTAT |
| | | GCTGACAGTGTTAAAGGCCGGTTCACCATCTCCAAGGATAAGGCTAAGGATACT |
| | | GTATACCTTCAGATGAACTCACTGAAACCTGAAGACACAGGAATGTATTCCTGT |
| | | AAGACGAAGGATGGCACCATCGCTACAATGGAGCTTTGCGATTTTGGCTATTGG |
| | | GGACAGGGCACACAGGTGACGGTTAGTAGC |
| hIL27 | 1367 | CAGGTGCAGCTCCAGGAGTCTGGTGGGGGTTCCGTGCAGGCCGGAGGCAGCCTG |
| Ra_VH | | CGCCTGTCCTGCCGCGCCTCTGGATCTACATACTCCAATTACTGTCTGGGGTGG |
| H11- | | TTCAGACAGAGTACTGGAAAAGAGCGCGAAGGTGTAGCCGTCATAAATTGGGTC |
| DR593 | | GGCGGTATGCTGTACTTCGCCGACAGCGTGAAGGGAAGGTTTACTGTGTCCCAG |
| | | GACCATGCTAAGAATACCGTGACCCTGCAAATGAACTCCCTTAAACCCGAGGAC |
| | | ACCGCTATGTACTATTGCGCGGCTGAGTCCGTGTCCAGCTTTTCTTGCGGAGGC |
| | | TGGCTCACCCGTCCAGGCCGTGTTCCTTATTGGGGACAGGGCACCCAGGTGACG |
| | | GTCTCCTCTGGTGGAGGTTCCCAGGTGCAGCTCCAGGAGAGTGGAGGGGGGTCA |
| | | GTACAGGCTGGCGGAAGCCTCCGCTTGAGCTGCGCTGCGTCCGGCTATCCCTAC |
| | | TCAAACGGCTATATGGGATGGTTTCGCCAGGCCCCCGGAAAGGAACGCGAGGGG |
| | | GTGGCCACCATTTATACTGGCGATGGCCGGACCTACTATGCTGACTCTGTGAAA |
| | | GGCCGCTTTACCATCAGCCGTGACAACGCCAAGAACACTGTTGATTTGCAGATG |
| | | TCTAGCCTGAAGCCCGAAGACACCGCTATGTATTACTGTGCAGCTCGGGGTGCC |
| | | CCTCTGTACTCCTCTGGAAGCCCCCTCACTCGGGCCAGATACAACGTGTGGGGA |
| | | CAGGGGACCCAGGTCACCGTGTCAAGT |
| hIL27 | 1368 | CAGGTGCAGTTGCAGGAGTCTGGGGCGGGGTCCGTCCAGGCTGGCGGTTCCCTT |
| Ra_VH | | CGCCTGTCTTGCCGTGCCTCAGGCAGTACCTACAGCAACTACTGTCTCGGTTGG |
| H11- | | TTCCGCCAGTCCACGGGCAAGGAGCGCGAAGGTGTGGCTGTTATCAACTGGGTG |
| DR593 | | GGAGGTATGGTGTACTTTGCCGACTCCGTTAAGGGGCGCTTTACCGTGAGCCAA |
| | | GACCACGCGAAAAACACGGTAACTTTGCAGATGAACTCTCTTAAACCCGAAGAT |
| | | ACCGCTATGTATTACTGCGCGGCAGAGTCTGTGTCTTCCTTCTCCTGTGGCGGA |
| | | TGGCTGACCAGGCCTGGGCGCGTCCCGTACTGGGGTCAGGGCACTCAGGTGACA |
| | | GTCAGCTCCGGCGGAAGCGGTGGCTCCGGTGGCAGTGGGCAAGTGCAACTCCAG |
| | | GAGTCAGGAGGTGGGAGCGTTCAAGCGGGGGGTAGTCTTCGGCTGAGTTGTGCT |
| | | GCGAGTGGCTATCCGTACTCCAACGGCTATATGGGTTGGTTCCGCCAGGCCCCT |
| | | GGCAAGGAGCGGGAGGGCGTGGCCACTATCTACACCGGCGATGGCCGTACCTAT |
| | | TACGCCGACTCCGTGAAGGGACGGTTTACCATCTCACGCGACAACGCGAAGAAC |
| | | ACAGTTGACCTCCAGATGTCCTCACTGAAGCCAGAGGATACTGCCATGTATTAC |
| | | TGTGCTGCCCGCGCAGCCCCCTGTACTCTAGTGGTTCCCCTCTCACACAGAGCC |
| | | AGATACAACGTCTGGGGACAAGGCACACAGGTGACTGTGAGTTCG |
| hIL27 | 1369 | CAGGTACAGCTCCAGGAGTCAGGTGGCGGTTCCGTCCAGGCAGGAGGTAGTTTG |
| Ra_VH | | CGTCTGTCCTGTCGTGCCAGCGGATCTACCTACTCTAACTACTGCCTGGGATGG |
| H11- | | TTCCGCCAGTCTACCGGCAAGGAGAGAGAGGGGGTTGCTGTTATCAACTGGGTC |
| DR594 | | GGCGGGATGCTCTATTTCGCCGACTCTGTGAAAGGGCGCTTTACTGTGTCCCAG |
| | | GATCACGCTAAGAACACCGTAACGCTCCAGATGAACAGCCTGAAGCCTGAAGAC |
| | | ACAGCCATGTATTACTGTGCCGCTGAGAGCGTGTCCAGCTTCTCTTGTGGCGGA |
| | | TGGCTCACCCGCCCCGGTCGTGTGCCCTACTGGGGGCAGGGAACCCAGGTCACA |
| | | GTAAGCTCCGGCGGTGGCAGCCAGGTGCAGTTGCAGGAGTCTGGTGGGGGGTCC |
| | | GTTCAGGGGGGAGGCTCCCTGAGACTTAGCTGTGTTGCGTCCGCATCCACCTAC |
| | | TGCACTTACGATATGCACTGGTATCGCCAGGCTCCTGGCAAGGGCCGTGAGTTC |
| | | GTGTCCGCTATTGATTCCGACGGCACCACTCGCTATGCCGATAGTGTCAAGGGG |
| | | AGGTTTACCATCTCCCAAGGTACAGCGAAAAATACAGTTTACCTCCAGATGAAC |
| | | AGCTTGCAGCCCGAGGACACGGCTATGTATTACTGTAAGACGGTGTGCGTCGTG |
| | | GGCAGTCGGTGGTCCGATTACTGGGGCCAGGGGACCCAGGTTACTGTCTCCAGC |
| hIL27 | 1370 | CAGGTACAGCTTCAGGAGTCCGGCGGTGGCTCTGTCCAGGCTGGCGGTTCCCTC |
| Ra_VH | | AGGCTGTCTTGCAGGGCTTCTGGTTCCACCTACAGCAACTACTGTTTGGGATGG |
| H11- | | TTCCGCCAGTCCACAGGTAAGGAGCGCGAGGGAGTGGCGGTCATCAACTGGGTA |
| DR594 | | GGAGGCATGTTGTACTTCGCCGACTCTGTGAAAGGCAGATTCACTGTCAGCCAG |
| | | GACCATGCCAAGAACACAGTCACTTTGCAGATGAACTCTCTGAAACCAGAGGAC |
| | | ACTGCTATGTATTACTGTGCTGCCGAGTCAGTCAGCTCGTTCTCCTGCGGAGGG |
| | | TGGCTCACCAGACCTGGCCGCGTGCCGTACTGGGGCCAGGGGACGCAGGTGACC |
| | | GTGTCCAGTGGCGGGAGCGGAGGTTCTGGTGGCTCAGGCCAGGTTCAACTGCAA |
| | | GAGTCCGGTGGAGGTTCTGTGCAGGCCGGTGGGTCCCTGCGTCTGTCCTGCGTG |
| | | GCCTCTGCCTCCACTTACTGTACCTATGATATGCACCGGTATCGCCAGGCCCCA |
| | | GGCAAGGGCCGTGAGTTTGTGAGCGCCATTGACAGTGATGGCACCACTAGGTAT |
| | | GCCGATTCCGTGAAGGGTCGCTTCACCATCAGTCAGGGCACTGCCAAGAACACT |
| | | GTGTATCTCCAGATGAACAGCTTGCAGCCCGAGGACACCGCCATGTATTACTGG |
| | | AAGACCGTCTGCGTCGTGGGAAGCCGCTGGAGCGACTATTGGGGCCAGGGGACC |
| | | CAGGTGACCGTCTCATCC |
| hIL27 | 1371 | CAGGTGCAACTGCAAGAGTCCGGCGGAGGCTCCGTTCAGGCTGGGGGGTCTCTG |
| Ra_VH | | CGGCTCTCCTGCCGCGCCAGCGGCTCTACTTATAGCAACTACTGCCTGGGATGG |
| H11- | | TTCAGACAGAGTACAGGTAAGGAGAGGGAGGGAGTTCGCTGTATCAACCGGGTT |
| DR595 | | GGCGGTATGCTCTATTTCGCGGACTCCGTGAAGGGCCGGTTCACCGTGAGCCAG |
| | | GACCACGCCAAGAACACCGTGACACTCCAGATGAACAGCCTTAAACCTGAGGAC |
| | | ACCGCTATGTACTATTGCGCAGCCGAAAGTGTTAGCTCTTTTTCCTGTGGTGGC |
| | | TGGCTGACTCGCCCTGGGCGCGTTCCATATTGGGGTCAGGGCACCCAGGTGACG |
| | | GTTTCATCCGGCGGAGGCAGTCAAGTGCAGCTTCAGGAATCCGGGGGGGGATCT |
| | | GTGCAGGCTGGGGGCAGTCTGACCCTGTCCTGTGCTGCCAGCGAGTACGCCTAC |
| | | TCCACTTGCAATATGGGATGGTATCGCCAGGCACCGGGAAAGGAGCGTGAGCTG |
| | | GTCAGTGCATTTATCAGCGATGGCTCCACCTATTACGCCGATTCTGTTAAGGGC |
| | | CGCTTCACCATCACCCGCGACAACGCCAAAAACACCGTATATTTGCAGATGAAT |
| | | AGCTTGAAGCCCGAAGATACTGCCATTTATTACTGCCCTGCTAATTGCTACAGA |
| | | CGCCTGCGCAACTATTGGGGCCAAGGCACACAGGTGACAGTAAGCTCC |
| hIL27 | 1372 | CAGGTGCAGCTCCAGGAATCTGGCGGTGGGAGCGTCCAGGGGGGGCGGAGCCTC |
| Ra_VH | | CGCTTGAGCTGCCGCGCTAGTGGTAGCACCTACAGTAACTATTGCCTCGGGTGG |
| H11- | | TTCAGGCAGTCCACCGGCAAGGAGCGCGAAGGCGTTGCCGTCATTAACTGGGTT |
| DR595 | | GGAGGTATGCTGTACTTTGCCGACAGCGTAAAGGGTCGTTTTACGGTAAGTCAA |
| | | GATCATGCTAAGAACACTGTGACATTGCAGATGAACAGCCTGAAGCCGGAGGAT |
| | | ACTGCCATGTATTACTGCGCGGCTGAGTCTGTAAGCTCTTTCTCATGCGGTGGA |
| | | TGGCTCACCCGTCCGGGTCGCGTTCCGTACTGGGGTCAGGGCACGCAAGTTACC |
| | | GTTTCTAGCGGAGGCAGTGGGGGCTCTGGGGGTTCCGGCCAGGTCCAGCTTCAG |
| | | GAGTCTGGCGGTGGCTCCGTTCAAGCCGGTGGCTCTCTGACACTGTCCTGTGCC |
| | | GCTTCTGAGTATGCGTACAGCACTTGCAACATGGGATGGTATCGCCAGGCCCCC |
| | | GGAAAAGAGCGCGAGCTGGTTTCCGCATTTATTTCCGACGGCAGCACCTATTAC |
| | | GCCGATTCCGTGAAGGGAAGGTTCACTATTACCGGCGACAACGCAAAGAATACC |
| | | GTCTACTTGCAGATGAACTCCCTGAAGCCCGAGGACACCGCTATCTACTATTGC |
| | | TCCGCAAATTGCTACCGGCGTCTCCGCAACTACTGGGGCCAGGGTACGCAGGTG |
| | | ACAGTCAGCTCC |
| hIL27 | 1373 | CAGGTCCAGGTTCAGGAGAGCGGCGGTGGCTCCGTCCAGGCCGGAGGCTCTCTG |
| Ra_VH | | CGCTTGTCCTGCCGTGCCTCCGGTTCCACATACAGCAACTACTGTCTGGGGTGG |
| H11- | | TTCCGTCAGAGCACTGGGAAGGAGAGAGAGGGCGTGGCTGTCATCAACTGGGTT |
| DR596 | | GGCGGGATGCTGTACTTTGCGGATTCAGTCAAGGGAAGGTTCACTGTTAGCCAG |
| | | GACCACGCTAAGAACACCGTAACTCTCCAGATGAACTCCCTGAAGCCCGAGGAT |
| | | ACAGCCATGTATTACTGTGCTGCCGAGTCCGTGTCCAGCTTCTCCTGTGGTGGG |
| | | TGGCTCACTCGCCCAGGGAGAGTCCCATACTGGGGACAGGGGACCCAGGTCACA |
| | | GTGAGTTCCGGCGGAGGCTCTCAGGTGCAGCTGCAAGAGTCTGGCGGAGGTCTT |
| | | GTGCAGCCTGGCGGTTCCCTGCGTCTGAGTTGTACCGCATCTGGTCTCACTTTT |
| | | GACGATAGCGTCATGGGATGGTTCCGCCAGGCACCGGGGAAAGGCCGCGAGGCC |
| | | GTCTCCTGTATTAGTTCCTCTGGTGCCAACGCTTTCTACGCTGATTCCGTGAAA |
| | | GGACGGTTTACCATCTCCCGCGATAACGCCAAAAACACCCTGTATCTCCAGATG |
| | | AACTCTCTGAAGCCAGAGGACACTGCGACCTATTACTGTAAACGGGGTCATGCT |
| | | TGTGCCGGATATTACCCCATCCCCTATGACGATTACTGGGGACAGGGCACCCAA |
| | | GTTACCGTGTCTAGT |
| hIL27 | 1374 | CAGGTGCAGCTCCAGGAAAGCGGAGGGGGCTCAGTCCAGGCCGGAGGCTCTCTG |
| Ra_VH | | AGACTCTCTTGCCGTGCATCCGGCAGCACTTATTCCAACTATTGTCTGGGGTGG |
| H11- | | TTTAGGCAGAGCACAGGCAAGGAGCGCGAAGGTGTCGCAGTGATTAACTGGGTC |
| DR596 | | GGCGGGATGTTGTACTTCGCCGATAGCGTCAAGGGGCGGTTTACAGTGTCCCAG |
| | | GACCACGCCAAGAACACCGTGACCCTTCAGATGAATAGCCTGAAGCCCGAAGAT |
| | | ACAGCAATGTATTACTGTGCGGCTGAGTCCGTGTCTTCCFTCAGCTGGGGGGGC |
| | | TGGCTGACCAGACCTGGCCGCGTTCCGTACTGGGGACAGGGAACTCAGGTGACC |
| | | GTTTCCTCTGGCGGATCAGGAGGCTCCGGCGGTTCTGGCCAGGTGCAGTTGCAG |
| | | GAGTCTGGTGGAGGCCTGGTACAACCCGGCGGTTCTTTGCGCCTCTCATGCACT |
| | | GCGAGCGGTCTTACATTTGATGACTCTGTAATGGGCTGGTTCCGTCAGGCTCCC |
| | | GGCAAGGGACGTGAGGCCGTGAGCTGCATTTCCAGCTCCGGCGCGAATGCCTTT |
| | | TACGCTGATTCCGTGAAGGGAAGATTCACCATTAGTAGAGACAACGCTAAGAAC |
| | | ACCCTGTATTTGCAGATGAACTCTCTGAAACCTGAGGATACAGCTACCTATTAC |
| | | TGCAAGCGCGGTCACGGTTGTGCTGGCTATTACCCGATCCCTTACGATGACTAC |
| | | TGGGGACAGGGCACCCAAGTGACAGTCTCTTCC |
| hIL27 | 1375 | CAGGTGCAGCTTCAGGAGTCCGGCGGTGGCTCCGTGCAGGCGGGCGAATCCCTT |
| Ra_VH | | CGTCTGAGCTGCCGCGCTTCTGGCTCCACCTACTCTAACTATTGTCTGGGCTGG |
| H12- | | TTTCGTCAGATCACCGGCAAAGAGCGCGAGGGAGTGGCCGTTATAAATTGGGTA |
| DR591 | | GGCGGTATGCTGTACTTTGCTGACTCCGTGAAGGGGCGGTTCACAGTTTCCCAG |
| | | GATCAGGCAAAGAATACTGTTTATCTGGAAATGAACTCCCTGAAGCCTGAGGAC |
| | | ACTGCTATGTATTACTGCGCCACCGAGTCCGTGTCCAGGTTTTCATGTGGGGGT |
| | | TGGCTGACCCGCCCTGATCGGGTCCCTTACTGGGGTCAGGGGACACAGGTAACA |
| | | GTGTCCAGTGGTGGCGGGTCCCAGGTGCAACTCCAGGAATCTGGGGGCGGTTCC |
| | | GTTCAGGCTGGCGGGTCCCTTCGCCTGTCCTGCACCGCGTCCGGTGCTATCGCT |
| | | TCAGGTTATATCGACTCCCGTTGGTGTATGGCTTGGTTTAGACAAGCTCCTGGC |
| | | AAAGAACGCGAGGGGGTCGCTGCCATCTGGCCAGGAGGCGGTCTTACCGTCTAC |
| | | GCCGACAGCGTGAAGGGCCGGTTTACCATCTCACGTGACCACGCCAAGAACACA |
| | | CTGTACCTCCAGATGAATAACCTGAAGCCGGAAGATACGGCCATGTATTACTGT |
| | | GCAGCCGGAAGCCCCCGCATGTGCCCATCTCTTGAGTTCGGGTTTGACTATTGG |
| | | GGACAGGGAACCCAAGTCACTGTGTCCTCT |
| hIL27 | 1376 | CAGGTCCAGGTCCAGGAGTCAGGAGGGGGCAGCGTACAGGCCGGAGAGTCCCTG |
| Ra_VH | | AGACTTAGTTGCCGCGCCAGGGGATCTACCTACTCTAACTACTGCCTCGGCTGG |
| H12- | | TTCAGGCAGATCACCGGCAAGGAGAGAGAGGGTGTGGCCGTAATCAACTGGGTT |
| DR591 | | GGGGGAATGCTGTACTTCGCCGACTCCGTGAAGGGTAGGTTCACTGTTTCTCAG |
| | | GACCAGGCAAAGAATACTGTGTATCTGGAGATGAACTCCCTGAAACCCGAAGAT |
| | | ACTGCCATGTATTACTGTGCCACCGAGAGCGTGTCCTCTTTCTCCTGTGGAGGC |
| | | TGGCTCACGCGCCCTGATCGCGTGCCCTATTGGGGACAGGGTACTCAAGTCACC |
| | | GTGTCAAGCGGAGGCAGCGGGGGCTCTGGAGGCAGCGGTCAGGTCCAGCTGCAA |
| | | GAGAGCGGTGGAGGTTCAGTACAGGCTGGGGGGTCACTGCGTCTGAGTTGTACT |
| | | GCCAGTGGAGCCATCGCGTCTGGCTACATCGACTCTAGGTGGTGCATGGCCTGG |
| | | TTCCGGCAGGCTCCTGGGAAGGAGAGAGAGGGAGTGGCCGCGATTTGGCCAGGT |
| | | GGGGGCCTGACCGTCTACGCCGATAGCGTCAAGGGGCGGTTCACCATCAGCCGC |
| | | GATCACGCTAAAAATACCCTGTACCTTCAGATGAATAACCTGAAGCCAGAAGAC |
| | | ACAGCTATGTACTATTGTGCCGCTGGGTCTCCCCGGATGTGTCCAAGTCTGGAA |
| | | TTTGGCTTCGACTACTGGGGGCAGGGCACACAGGTTACTGTGAGTTCC |
| hIL27 | 1377 | CAGGTCCAGTTGCAGGAGTCCGGCGGTGGCTCCGTGCAGGCCGGTGAGTCTCTG |
| Ra_VH | | AGACTGTCTTGCCGCGCTAGTGGGTCCACATACAGTAACTACTGCCTGGGCTGG |
| H12- | | TTCCGCCAGATCACTGGTAAGGAAAGGGAAGGCGTTGCTGTTATCAACTGGGTG |
| DR592 | | GGCGGTATGCTGTACTTCGCTGACTCCGTCAAGGGCCGGTTCACCGTCTCTCAG |
| | | GATCAGGCTAAGAATACAGTCTATCTGGAAATGAACAGCCTGAAGCCCGAAGAT |
| | | ACTGCTATGTACTATTGTGCTACCGAGTCTGTCTCTAGCTTTTCTTGCGGAGGC |
| | | TGGCTGACCCGCCCGGACAGGGTCCCTTACTGGGGCCAGGGCACTCAGGTGACC |
| | | GTATCTAGCGGTGGGGGTTCACAGGTGCAACTCCAGGAGAGCGGTGGGGGATCT |
| | | GTGCAGGCCGGAGGCAGCCTTCGCTTGAGCTGCACGGCCCCCGGCTTTACCAGT |
| | | AACAGCTGTGGCATGGACTGGTATCGCCAGGCCCCCGGAAAGGAGCGCGAGTTC |
| | | GTCAGCTCCATCTCCACTGACGGAACTACCGGATATGCTGACTCTGTCAAAGGT |
| | | CGCTTCACCATCTCTAAGGATAAGGCAAAGGACACAGTCTATCTGCAAATGAAC |
| | | AGTCTCAAGCCAGAGGACACAGGTATGTACTCGTGCAAGACCAAGGATGGCACT |
| | | ATTGCCACAATGGAACTTTGTGATTTCGGCTACTGGGGACAGGGCACCCAAGTT |
| | | ACTGTTTCTAGC |
| hIL27 | 1378 | CAGGTGCAACTTCAGGAATCAGGGGGTGGCTCCGTGCAGGCTGGTGAGTCACTG |
| Ra_VH | | CGTCTTTCTTGCAGGGCCTCTGGGTCCACCTACTCCAACTACTGCCTGGGTTGG |
| H12- | | TTCAGGCAGATCACCGGCAAGGAGAGAGAGGGCGTGGCCGTTATAAATTGGGTA |
| DR592 | | GGAGGTATGCTGTACTTCGCCGACAGCGTCAAGGGACGCTTCACTGTCAGTCAG |
| | | GATCAGGCCAAGAACACTGTGTACCTTGAGATGAACTCTCTGAAGCCTGAAGAC |
| | | ACAGCCATGTATTACTGTGCCACAGAGAGTGTCTCTAGCTTCAGCTGGGGTGGA |
| | | TGGCTGACCCGTCCTGACCGCGTTCCCTACTGGGGCCAGGGCACTCAGGTTACT |
| | | GTGTCTAGCGGAGGGAGCGGAGGGTCAGGTGGCTCTGGCCAGGTCCAGTTGCAG |
| | | GAGTCTGGTGGGGGCTCTGTGCAAGCTGGCGGGTCTCTGAGACTGTCCTGTACC |
| | | GCACCCGGCTTCACATCCAATAGCTGCGGCATGGACTGGTATAGACAAGCGCCT |
| | | GGGAAGGAGCGCGAGTTTGTTAGCTCCATCAGCACAGACGGCACAACGGGCTAT |
| | | GCCGACTCTGTGAAAGGGCGCTTTACCATTTCTAAGGACAAGGCCAAGGACACC |
| | | GTCTATCTCCAGATGAACAGCCTGAAGCCTGAGGACACAGGCATGTATAGCTGC |
| | | AAAACGAAGGACGGGACCATCGCAACGATGGAGCTGTGTGACTTTGGGTACTGG |
| | | GGGCAGGGCACCCAGGTTACCGTCTCTTCC |
| hIL27 | 1379 | CAGGTGCAACTCCAGGAGAGCGGTGGAGGTAGCGTGCAAGCTGGTGAGAGTCTG |
| Ra_VH | | CGCCTCAGCTGCCGCGCTTCCGGCTCAACTTACAGCAATTACTGCCTGGGTTGG |
| H12- | | TTCCGTCAGATCACCGGAAAGGAGAGAGAGGGGGTAGCAGTCATCAACTGGGTC |
| DR593 | | GGCGGAATGCTCTATTTTGCCGACTCCGTTAAAGGACGCTTCACCGTGTCTCAA |
| | | GACCAGGCTAAGAACACAGTGTATCTGGAGATGAACTCTCTCAAGCCAGAGGAT |
| | | ACCGCCATGTACTATTGCGCCACTGAATCCGTGTCTTCGTTCTCATGTGGGGGG |
| | | TGGCTCACTCGTCCTGATCGGGTGCCATACTGGGGGCAGGGCACCCAGGTCACC |
| | | GTCTCTAGTGGTGGAGGCAGCCAGGTCCAGTTGCAGGAGTCTGGGGGCGGTTCT |
| | | GTCCAAGCTGGTGGCTCACTCCGTCTGTCCTGCGCCGCGAGCGGGTATCCATAC |
| | | TCAAATGGCTACATGGGATGGTTCCGTCAGGGTCCAGGAAAGGAGCGTGAGGGG |
| | | GTCGCGACCATTTATACCGGCGACGGACGCACCTACTATGCCGACTCCGTGAAG |
| | | GGGAGGTTTACCATCAGTCGCGATAACGCCAAGAACACCGTGGATCTCCAGATG |
| | | AGTTCCCTGAAGCCTGAAGACACCGCTATGTATTACTGCGCGGCACGTGCCGCA |
| | | CCGGTTTACAGTAGCGGCAGTCCCCTGACCCGCGCGAGGTATAACGTGTGGGGC |
| | | CAGGGCACCCAGGTTACAGTGTCTTCT |
| hIL27 | 1380 | CAGGTGCAGCTCCAGGAGTCTGGGGGGGGTTCTGTCCAAGCTGGCGAGAGCCTG |
| Ra_VH | | CGCCTGTCCTGCCGGGCTTCCGGTTCCACCTATTCTAACTATTGTCTGGGTTGG |
| H12- | | TTTCGCCAGATCACTGGAAAGGAGAGGGAGGGTGTGGCAGTTATCAACTGGGTG |
| DR593 | | GGTGGGATGGTCTATTTCGCCGATTCAGTTAAGGGACGCTTCACAGTGAGCCAA |
| | | GACCAGGCTAAGAACACAGTCTACCTGGAGATGAACAGCCTGAAACCGGAAGAC |
| | | ACGGCAATGTATTACTGCGCGACCGAGAGCGTGTCTAGTTTTAGCTGTGGGGGC |
| | | TGGCTGACGAGACCCGACAGGGTGCCTTACTGGGGGCAGGGTACTCAGGTTACC |
| | | GTCTCTTCCGGTGGCAGTGGTGGCAGCGGAGGCAGCGGCCAGGTGCAGCTGCAA |
| | | GAATCTGGCGGTGGCAGCGTTCAGGCGGGAGGTTCCCTTCGCCTGTCTTGTGCT |
| | | GCATCCGGCTACCCTTACTCTAACGGTTACATGGGCTGGTTTCGTCAAGCCCCC |
| | | GGCAAGGAACGTGAGGGGGTGGCGACAATTTATACAGGTGATGGCCGCACTTAT |
| | | TACGGTGACTCCGTTAAGGGACGCTTCACCATCTCCCGCGATAACGCCAAGAAT |
| | | ACGGTGGACCTCCAGATGTCCTCTCTCAAACCTGAGGACACCGCAATGTACTAT |
| | | TGCGCCGCTCGCGCCGCGCCGCTCTACAGTAGCGGCAGCCCACTGACTCGCGCC |
| | | CGCTACAATGTGTGGGGACAGGGAACCCAGGTGACCGTGAGCAGT |
| hIL27 | 1381 | CAAGTGCAGGTCCAAGAGTCCGGTGGAGGCTCCGTGCAGGCTGGCGAGTCCCTG |
| Ra_VH | | CGTCTGTCCTGCCGCGCGTCTGGTAGTACTTACTCTAACTATTGTCTCGGTTGG |
| H12- | | TTCAGGCAGATCACCGGCAAAGAGCGCGAAGGAGTTGCCGTTATCAACTGGGTG |
| DR594 | | GGCGGTATGCTCTATTTCGCCGACAGCGTGAAAGGGAGATTCACTGTGAGCCAG |
| | | GACCAAGCTAAGAACACAGTGTATGTTGAGATGAACAGCCTCAAGCCTGAGGAC |
| | | ACCGCTATGTATTACTGTGCCACAGAATCTGTGTCTTCCTTCTCATGTGGGGGT |
| | | TGGCTCACCAGGCCGGACAGGGTCCCATACTGGGGCCAGGGCACCCAGGTGACC |
| | | GTGTCTAGCGGAGGTGGCAGCCAGGTGCAGTTGCAGGAGTCCGGTGGGGGCTCC |
| | | GTGCAGGCTGGAGGCTCACTTCGGCTCAGCTGCGTGGCCTCAGCGTCTACCTAC |
| | | TGCACATACGATATGCACTGGTATCGTCAAGCACCCGGCAAAGGCCGCGAGTTC |
| | | GTCAGCGCCATTGATTCCGACGGTACAACCCGTTACGCTGACTCCGTCAAGGGG |
| | | CGTTTTACCATTTCCCAGGGGACCGCTAAGAACACCGTTTATCTTCAGATGAAC |
| | | AGCCTCCAGCCGGAAGACACAGCCATGTATTACTGTAAAACCGTGTGCGTAGTG |
| | | GGCTCCAGATGGTCAGACTATTGGGGGCAGGGCACCCAGGTAACCGTGTCCTCT |
| hIL27 | 1382 | CAGGTTCAGGTTCAGGAATCAGGAGGTGGCTCAGTGCAGGCAGGAGAGTCTCTC |
| Ra_VH | | CGCCTGTCCTGTCGTGCCAGCGGATCTACGTATTCCAACTACTGCCTCGGTTGG |
| H12- | | TTCAGGCAGATCACCGGCAAAGAGCGCGAAGGAGTGGGGGTTATCAACTGGGTT |
| DR594 | | GGCGGAATGCTGTACTTCGCAGACAGTGTCAAGGGCAGATTCACTGTGTCCCAG |
| | | GACCAGGCCAAAAACACAGTGTACCTGGAGATGAATAGTTTGAAGCCGGAAGAT |
| | | ACAGCCATGTATTACTGCGCCACCGAGTCTGTGTCCAGCTTTTCTTGCGGAGGC |
| | | TGGCTGACCCGCCCAGACCGCGTCCCCTACTGGGGCCAGGGAACTCAAGTCACC |
| | | GTGTCTTCCGGGGGAGTGGTGGCAGGGGGcGGCTCCGGCCAGGTGCAGTTGCAG |
| | | GAGTCCGGTGGGGGAAGTGTCCAGGCCGGGGGCTCCTTGAGACTTTCATGCGTC |
| | | GCCTCTGCCTCCACCTACTGCACTTACGACATGCACTGGTATCGCCAGGCTCCG |
| | | GGTAAGGGACGGGAGTTCGTATCTGCCATCGACTCCGACGGGACCACACGCTAC |
| | | GCGGACAGCGTGAAAGGCAGGTTCACCATCAGTCAGGGGACCGCCAAGAATACG |
| | | GTTTACCTCCAGATGAACTCTCTTCAGCCGGAGGACACGGCTATGTATTACTGC |
| | | AAAACTGTGTGCGTGGTTGGAAGCAGATGGTCTGATTACTGGGGTCAGGGCACC |
| | | CAGGTGACTGTATCAAGC |
| hIL27 | 1383 | CAAGTGCAGCTGCAAGAAAGTGGCGGTGGCAGTGTGCAAGCTGGAGAGTCCCTC |
| Ra_VH | | AGACTGAGTTGTAGAGCCAGTGGTAGCACATATAGCAATTACTGCCTGGGTTGG |
| H12- | | TTTCGTCAGATCACCGGCAAGGAGAGAGAAGGCGTGGCCGTCATAAATTGGGTC |
| DR595 | | GGCGGTATGGTGTACTTTGCAGATTCCGTCAAGGGCCGCTTTACGGTCAGCCAA |
| | | GATCAGGCCAAGAATACTGTGTATCTGGAAATGAACAGTCTGAAACCGGAGGAC |
| | | ACCGCTATGTATTACTGCGCTACTGAATCCGTCTCCTCTTTTAGTTGCGGTGGG |
| | | TGGCTCACCAGACCGGATCGCGTGCCCTATTGGGGCCAGGGCACCCAGGTGACG |
| | | GTGTCCTCAGGAGGCGGATCACAGGTGCAGCTCCAGGAGAGCGGGGGGGGGAGT |
| | | GTGCAGGCCGGTGGCAGCCTGACCTTGTCATGTGCGGCTAGTGAGTACGCCTAT |
| | | TCTACCTGTAATATGGGCTGGTATCGCCAGGCACCCGGAAAAGAGCGCGAGCTG |
| | | GTGTCCGCGTTCATTAGCGATGGGTCTACCTATTACGCCGATTCCGTGAAGGGC |
| | | AGGTTCACCATCACACGCGACAACGCTAAGAACACCGTATACCTCCAGATGAAC |
| | | AGCCTGAAGCCAGAAGACACGGCCATCTATTACTGCTCTGCTAATTGCTACGGC |
| | | AGGCTGCGCAACTACTGGGGACAGGGCACTCAGGTGACTGTGTCTAGT |
| hIL27 | 1384 | CAGGTGCAACTTCAGGAGTCCGGTGGGGGTAGTGTGCAGGCTGGCGAGTCTCTG |
| Ra_VH | | CGTCTGTCTTGCCGGGCGTCCGGCTCCACCTACTCCAACTACTGCCTGGGTTGG |
| H12- | | TTCCGCCAGATTACCGGCAAAGAAAGGGAGGGCGTTGCGGTTATTAACTGGGTG |
| DR595 | | GGCGGGATGCTGTACTTCGCAGATTCTGTGAAGGGTAGGTTTACAGTCTCACAG |
| | | GACCAGGCTAAGAACACGGTGTACCTGGAAATGAACAGTCTCAAGCCCGAAGAC |
| | | ACCGCCATGTATTACTGCGCTACAGAGTCTGTTTCCTCTTTTAGCTGGGGTGGG |
| | | TGGCTGACCCGCCCTGACCGTGTGCCTTACTGGGGACAGGGGACCCAGGTCACT |
| | | GTAAGCTCTGGAGGGTCCGGTGGCTCCGGCGGGAGCGGCCAGGTACAGCTCCAG |
| | | GAGTCCGGGGGGGGAAGTGTGCAGGCCGGAGGCAGCCTCACCCTGAGCTGCGCG |
| | | GCATCCGAGTACGCCTATTCTACCTGTAACATGGGGTGGTACAGGCAGGCTCCG |
| | | GGCAAGGAGAGAGAGTTGGTTTCTGCTTTTATCAGTGATGGCAGTACCTATTAC |
| | | GCGGATTCCGTGAAAGGGAGATTCACCATTACACGTGACAACGCTAAGAACACC |
| | | GTTTATTTGCAGATGAACTCCCTCAAGCCCGAGGATACAGCCATTTACTATTGC |
| | | TCTGCCAACTGCTATCGTCGCCTGCGCAACTACTGGGGACAGGGGACACAGGTG |
| | | ACCGTGTCCAGT |
| hIL27 | 1385 | CAGGTCCAGCTCCAGGAGTCCGGGcGGGGCTCCGTGCAGGCTGGCGAATCTTTG |
| Ra_VH | | CGCCTCTCATGCAGAGCTTCCGGCTCCACCTATAGCAACTATTGTCTGGGTTGG |
| H12- | | TTTCGCCAGATCACCGGCAAAGAGCGTGAAGGGGTCGCCGTCATCAACTGGGTC |
| DR596 | | GGCGGGATGCTGTACTTTGCGGACTCTGTTAAGGGTCGTTTCACAGTCAGTCAG |
| | | GATCAAGCGAAGAACACCGTGTACCTGGAGATGAACTCCCTGAAGCCGGAGGAC |
| | | ACCGCAATGTATTACTGTGCCACTGAATCTGTGAGCAGTTTTAGCTGTGGCGGT |
| | | TGGCTGACCCGCCCTGACCGCGTTCCCTACTGGGGGCAGGGCACCCAAGTAACC |
| | | GTGTCTTCCGGGGGGGGGTCTCAGGTCCAGCTCCAGGAGTCTGGAGGGGGCCTT |
| | | GTCCAGCCCGGAGGCAGCCTGCGCCTGTCTTGCACGGCTAGTGGCCTGACATTC |
| | | GACGATAGCGTGATGGGCTGGTTCAGACAGGCTCCGGGGAAGGGCCGTGAGGCC |
| | | GTTTCTTGTATCTCTAGCTCTGGTGCTAACGCCTTCTATGCCGACTCCGTGAAG |
| | | GGCCGCTTCACAATCAGCCGTGACAACGCCAAGAACACGCTGTATCTTCAGATG |
| | | AACTCCCTGAAGCCTGAGGATACCGCCACATATTACTGTAAGCGCGGTCACGCC |
| | | TGTGCAGGCTATTACCCCATCCCCTACGATGACTACTGGGGTCAGGGAACCCAG |
| | | GTTACGGTTTCATCT |
| hIL27 | 1386 | CAGGTGCAACTTCAGGAGAGCGGGGGCGGGAGCGTGCAGGGGGGGGAGAGCCTG |
| Ra_VH | | CGCCTGTCCTGCCGTGCGTCTGGCTCCACCTACAGTAACTACTGCCTGGGGTGG |
| H12- | | TTTCGCCAGATTACGGGCAAGGAGCGCGAAGGTGTGGCCGTTATCAACTGGGTG |
| DR596 | | GGTGGAATGCTCTACTTTGCCGATTCTGTGAAAGGACGGTTTACCGTGTCTCAG |
| | | GATCAGGCTAAGAATACAGTTTACCTGGAGATGAACTCTCTCAAGCCTGAGGAT |
| | | ACAGCGATGTACTATTGTGCAACCGAGTCCGTCTCTTCCTTCAGCTGCGGGGGG |
| | | TGGCTGACCCGTCCTGACCGCGTGCCATACTGGGGTCAAGGTACACAGGTGACA |
| | | GTCAGCTCTGGTGGCAGCGGCGGTAGCGGGGGCAGTGGCCAGGTCCAGTTGCAG |
| | | GAAAGTGGAGGCGGTCTGGTTCAACCTGGGGGCTCCCTGCGCCTGTCTTGCACA |
| | | GCCTGTGGACTGACGTTTGATGACTCCGTGATGGGCTGGTTCAGACAGGGTCCC |
| | | GGTAAGGGCCGCGAGGCCGTCAGCTGTATCAGTAGCTCCGGGGCCAACGCTTTC |
| | | TATGCAGACTCTGTCAAAGGCAGATTCACTATCAGCCGCGACAACGCCAAGAAC |
| | | ACACTGTATTTGCAGATGAACAGCCTGAAACCTGAAGATACTGCTACATACTAT |
| | | TGTAAGCGCGGCCATGCCTGTGCTGGGTATTACCCTATCCCCTACGATGACTAC |
| | | TGGGGCCAGGGCACGCAGGTTACAGTAAGCAGT |
| hIL27 | 1387 | CAGGTGCAGCTCCAGGAATCCGGTGGGGGGTCCGTGCAGGCTGGCGGTTCCCTC |
| Ra_VH | | CGTCTGTCCTGTGTGGCCTCCGGGTACGTCAGCTGTGACTACTTTCTGCCATCC |
| H13- | | TGGTATCGCCAGGCCCCCGGTAAGGAGAGAGAGTTCGTGTCTATCATTGATGGC |
| DR591 | | ACTGGCAGCACTTCTTACGCCGCATCTGTGAAGGGGCGGTTCACAGCCTGCCAG |
| | | GATAGAGGCAAAAACATTGCCTACCTTCAGATGAACTCTTTGAAACCCGAGGAT |
| | | ACTGCCATGTATTACTGCAAAGCGAGCTGTGTCCGGGGCCGCACCATCTCCGAA |
| | | TACTGGGGACAGGGAACCCAGGTCACGGTCTCGTCTGGTGGGGGGTCCCAGGTG |
| | | CAGCTTCAGGAGAGGGGGGGAGGTTCCGTGCAGGCCGGAGGCTCTCTCAGGCTC |
| | | AGCTGCACAGCGTCCGGCGCTATTGCCAGCGGATATATCGACAGTCGTTGGTGT |
| | | ATGGCCTGGTTTCGCCAGGCTCCGGGCAAGGAGCGTGAGGGAGTCGCTGCCATT |
| | | TGGCCCGGGGGGGGACTGACCGTGTATGCGGACTCTGTGAAAGGAAGATTCACC |
| | | ATCTCTCGGGATCACGCTAAGAATACCCTGTATCTCCAGATGAATAACCTGAAG |
| | | CCCGAGGACACCGCCATGTATTACTGCGCCGCAGGATCACCCCGCATGTGTCCG |
| | | TCTCTGGAGTTCGGTTTCGACTACTGGGGCCAAGGCACCCAAGTCACTGTGTCT |
| | | TCA |
| hIL27 | 1388 | CAGGTGCAACTCCAGGAGTCCGGGCGGGGCTCCGTACAGGCTGGAGGCAGCCTC |
| Ra_VH | | CGTCTCAGTTGTGTTGCCAGCGGCTATGTAAGCTGCGACTATTTCCTGCCTTCC |
| H13- | | TGGTATCGCCAGGCACCTGGGAAGGAGCGTGAGTTCGTGTCCATCATTGATGGC |
| DR591 | | ACAGGCAGCACCTCCTATGCCGCTTCAGTCAAGGGACGGTTTACAGCCTCCCAG |
| | | GACCGTGGCAAGAACATCGCCTACCTTCAGATGAACTCCCTGAAGCCTGAGGAC |
| | | ACAGCCATGTATTACTGTAAGGCTTCCTGTGTGCGTGGCCGGACCATCTCTGAA |
| | | TACTGGGGACAGGGCACGCAGGTGACGGTCAGCTCTGGGGGTAGTGGGGGGTCC |
| | | GGCGGGAGTGGACAGGTGCAGCTTCAGGAGAGCGGTGGGGGATCTGTCCAAGCC |
| | | GGTGGCAGCCTTAGGCTCTCTTGCACTGCCAGCGGGGCCATCGCTTCAGGATAT |
| | | ATTGATAGCCGCTGGTGCATGGCCTGGTTCCGCCAGGCCCCAGGGAAAGAGCGT |
| | | GAAGGCGTGGCAGCCATCTGGCCGGGTGGCGGTCTGACCGTCTACGCCGACTCT |
| | | GTTAAAGGAAGATTTACCATTTCCCGCGATCACGCTAAGAACACTCTGTATCTT |
| | | CAAATGAATAACCTGAAACCTGAAGACACCGCTATGTATTACTGTGCTGCCGGT |
| | | AGCCCGAGGATGTGCCCAAGCCTTGAGTTCGGCTTCGACTATCGGGGCCAGGGA |
| | | ACTCAAGTGACCGTGAGCAGC |
| hIL27 | 1389 | CAGGTGCAGCTCCAGGAGTCTGGTGGGGGTTCCGTCCAGGCTGGCGGTTCCCTG |
| Ra_VH | | AGGCTCTCATGCGTGGCCAGCGGTTACGTGTCTTGCGACTATTTCCTGCCCTCA |
| H13- | | TGGTATCGTCAGGCCCCTGGCAAGGAGCGCGAGTTCGTGTCAATTATCGACGGC |
| DR592 | | ACAGGCAGTACCAGCTATGCAGCCAGCGTTAAGGGCCGTTTTACCGCGTCACAG |
| | | GATCGCGGGAAGAATATCGCCTACCTCCAGATGAACAGTCTGAAACCTGAAGAT |
| | | ACAGCGATGTATTACTGTAAGGCCAGCTGTGTGAGGGGTCGGACGATCTCTGAA |
| | | TACTGGGGCCAAGGAACCCAAGTAACTGTGTCCAGCGGAGGGGGATCTCAGGTG |
| | | CAGCTTCAGGAATCCGGCGGTGGCAGCGTCCAGGCCGGAGGGAGCCTGCGTCTT |
| | | TCCTGCACCGCTCCTGGCTTCACCAGCAACAGTTGGGGGATGGATTGGTATCGT |
| | | CAGGCTCCCGGCAAGGAACGGGAGTTCGTGTCCAGTATCAGCACTGATGGCACT |
| | | ACCGGATACGCTGATTCCGTTAAGGGTCGTTTCACCATCACCAAAGATAAGGCC |
| | | AAAGACACCGTGTATGTCCAGATGAACTCCCTGAAACCAGAAGACACAGGTATG |
| | | TATTCTTGTAAGACTAAAGACGGCACCATCGCGACAATGGAGCTGTGTGATTTC |
| | | GGATACTGGGGTCAGGGAACACAGGTGACTGTGTCCTCT |
| hIL27 | 1390 | CAAGTACAGTTGCAGGAGTCTGGCGGAGGCTCCGTGCAGGGGGGCGGGAGCCTG |
| Ra_VH | | AGGCTGAGTTGTGTGGCCTCAGGGTATGTGTCGTGTGACTACTTTCTCCCGAGC |
| H13- | | TGGTATCGCCAGGCTCCAGGGAAGGAGCGTGAGTTCGTGTCTATTATCGACGGG |
| DR592 | | ACTGGATCTACCTCTTATGCCGCATCTGTCAAGGGGCGCTTCACCGCCTCTCAG |
| | | GATCGCGGAAAGAACATCGCCTACTTGCAAATGAACAGCCTGAAGCCCGAGGAC |
| | | ACTGCCATGTATTACTGTAAAGCCTCTTGTGTGAGAGGCCGCACTATTTCAGAG |
| | | TACTGGGGACAGGGAACCCAGGTCACTGTCAGCAGTGGAGGTTCTGGCGGTTCT |
| | | GGTGGGTCAGGACAGGTACAGTTGCAGGAAAGCGGAGGCGGTTCCGTGCAGGCC |
| | | GGAGGCTCCCTGAGGCTGTCTTGCACAGCTCCTGGTTTCACATCTAACAGTTGC |
| | | GGCATGGACTGGTATCGCCAGGCCCCAGGGAAGGAGAGGGAATTTGTGTCTAGC |
| | | ATCAGCACCGACGGAACCACAGGATACGCCGACTCCGTCAAGGGTCGTTTTACT |
| | | ATCAGTAAGGACAAGGCTAAGGATACTGTCTACCTGCAAATGAACAGTCTGAAG |
| | | CCCGAGGACACCGGCATGTATAGCTGTAAGACTAAGGATGGAACTATTGCGACT |
| | | ATGGAGCTGTGTGACTTCGGGTATTGGGGCCAGGGTACACAAGTAACTGTGAGT |
| | | TCT |
| hIL27 | 1391 | CAGGTACAGCTCCAGGAGAGCGGTGGCGGTTCCGTCCAAGCAGGCGGTTCCCTC |
| Ra_VH | | AGACTGTCATGCGTCGCAAGCGGCTACGTGTCATGTGATTACTTCCTGCCCTCC |
| H13- | | TGGTACAGGCAAGCGCCAGGAAAGGAAAGGGAGTTTGTCTCCATTATCGACGGC |
| DR593 | | ACCGGAAGCACAAGTTACGCCGCGTCTGTTAAGGGCCGGTTCACCGGTTCTCAA |
| | | GATCGCGGCAAAAACATCGCCTACCTGCAAATGAACAGCCTCAAACCAGAGGAC |
| | | ACCGCGATGTATTACTGCAAGGCTTCTTGTGTGCGTGGTCGGACGATTTCCGAA |
| | | TACTGGGGCCAAGGCACCCAGGTGACTGTTTCTAGCGGTGGGGGCAGCCAGGTC |
| | | CAGTTGCAGGAGAGTGGCGGTGGCAGCGTTCAGGCTGGGGGGAGCCTGAGACTC |
| | | AGCTGCGCTGCGTCTGGTTACCCCTATTCCAACGGCTATATGGGGTGGTTCAGA |
| | | CAGGCACCAGGGAAGGAGCGCGAGGGGGTGGCCACCATCTACACCGGCGATGGC |
| | | CGCACATATTACGCTGATTCAGTGAAGGGAAGATTCACTATCAGTAGGGACAAC |
| | | GCTAAGAATACCGTGGATCTCCAGATGAGTTCACTGAAGCCGGAGGATACCGCG |
| | | ATGTATTACTGCGCGGCTCGCGCAGCCCCTCTCTACAGTAGCGGCTCCCCTCTT |
| | | ACGCGGGCTAGATACAACGTGTGGGGCCAGGGAACCCAGGTTACTGTGAGCAGT |
| hIL27 | 1392 | CAGGTTCAGCTGCAAGAGTCCGGCGGGGGCTCCGTTCAGGCCGGAGGCTCTCTC |
| Ra_VH | | AGGCTGAGCTGTGTCGCCTCCGGCTATGTGAGGTGTGACTACTTTCTGCCATCC |
| H13- | | TGGTATCGCCAGGCCCCTGGCAAGGAACGCGAGTTTGTCTCCATCATTGACGGC |
| DR593 | | ACCGGCTCCACATCCTACGCGGCTTCCGTGAAGGGGCGTTTCACCGCGTCCCAA |
| | | GATCGCGGGAAGAATATCGCCTACCTCCAAATGAACTCTCTCAAGCCGGAGGAC |
| | | ACTGGTATGTATTACTGTAAGGCATCCTGTGTTCGGGGGGGGACAATCAGCGAA |
| | | TACTGGGGCCAGGGCACCCAGGTAACTGTATCCAGCGGAGGCTCTGGAGGTTCC |
| | | GGTGGCTCCGGGCAGGTCCAGGTCCAGGAGTCTGGCGGTGGCTCCGTGCAGGCT |
| | | GGTGGCAGCCTTCGTCTGTCTTGCGCTGCCTCTGGTTATCCTTACTCCAATGGC |
| | | TACATGGGCTGGTTTCGCCAGGCCCCAGGGAAGGAGAGGGAGGGCGTGGCCACT |
| | | ATCTACACCGGCGATGGCCGCACCTACTATGCTGACAGCGTGAAGGGCAGATTC |
| | | ACCATCAGCAGGGATAACGCAAAGAACACAGTGGACCTTCAGATGAGTAGCCTG |
| | | AAGCCAGAGGACACCGCTATGTATTACTGTGCGGCCAGGGCAGCTCCGTTGTAC |
| | | TCTTCCGGTTCCCCTCTGACCAGAGCGAGATATAATGTGTGGGGACAGGGCACC |
| | | CAGGTTACCGTCAGTTCC |
| hIL27 | 1393 | CAAGTGCAGTTGCAGGAGAGCGGGGCGGGGTCCGTCCAGGCTGGGGGCTCACTG |
| Ra_VH | | CGTCTGAGCTGTGTGGCCTCTGGTTACGTGTCCTGTGACTATTTCCTGCCTTCT |
| H13- | | TGGTATAGACAGGCCCCAGGCAAAGAAAGGGAGTTCGTGTCTATTATCGACGGC |
| DR594 | | ACCGGCAGCACTAGCTATGCGGCCAGCGTTAAGGGGCGTTTCACAGCCAGCCAG |
| | | GATCGCGGCAAGAACATCGCCTATTTGCAGATGAACTCGTTGAAGCCCGAGGAC |
| | | ACCGCCATGTACTATTGCAAGGCCAGCTGCGTGGGGGGCAGGACCATTTCCGAG |
| | | TACTGGGGCCAAGGCACTCAGGTGACAGTGTCTTCCGGTGGCGGTAGTCAGGTG |
| | | CAGTTGCAGGAGTCGGGGGGAGGCAGCGTGCAGGCCGGTGGGTCCCTCCGCCTG |
| | | AGCTGTGTGGCGTCCGCAAGCACCTACTGTACCTATGATATGCACTGGTACAGG |
| | | CAAGCCCCTGGCAAGGGCCGCGAGTTTGTCCCTGCTATCGACCCAGACGGGACC |
| | | ACTCGCTACGCTGACAGTTGTAAAGGGGCGCTTCACTATAGCCAGGGCACAGCC |
| | | AAGAACACCGTGTACCTCCAGATGAATAGCCTCCAGCCCGAGGACACTGCCATG |
| | | TATTACTGCAAGACCGTATGCGTGGTTGGGAGTCGCTGGTCAGATTACTGGGGT |
| | | CAGGGTACTCAGGTGACCGTCTCCAGC |
| hIL27 | 1394 | CAGGTGCAGTTGCAGGAATCCGGCGGTGGCTCAGTCCAGGCCGGGGGCTCCCTG |
| Ra_VH | | AGGCTGTCATGCGTCGCCAGCGGATATGTGTCTTGCGACTATTTTCTGCCCTCC |
| H13- | | TGGTATCGCCAAGCTCCCGGCAAGGAGAGAGAATTTGTTTCTATCATTGAGGGG |
| DR594 | | ACAGGTTCCACTTCCTACGCTGCCAGCGTCAAAGGCCGGTTCACGGCGAGCCAG |
| | | GATAGGGGCAAAAACATCGCCTACCTGCAAATGAACTCTCTCAAGCCCGAGGAC |
| | | ACTGCCATGTATTACTGTAAAGCCAGCTGTGTGCGCGGTCGCACTATCTCTGAA |
| | | TATTGGGGCCAGGGCACCCAGGTGACCGTCTCCTCTGGAGGTTCAGGGGGCTCC |
| | | GGTGGCAGCGGTCAGGTCCAGCTCCAAGAGTCTGGGGGGGGTAGTGTGCAGGCG |
| | | GGCGGTTCCCTCAGACTCTCCTGCGTGGCCCCCGCCTCTACCTATTGCACCTAC |
| | | GATATGCACTGGTATCGGCAGGCTCCAGGAAAGGGCCGGGAGTTCGTGAGCGCG |
| | | ATTGACTCCGATGGCACTACCCGTTATGCCGATTCCGTTAAGGGACGGTTCACA |
| | | ATTAGCCAGGGAACCGCGAAGAACACCGTGTACCTGCAAATGAACTCTCTCCAG |
| | | CCGGAGGATACTGCCATGTATTACTGTAAGACTGTGTGCGTCGTTGGGTCCAGG |
| | | TGGTCCGATTATTGGGGCCAGGGAACTCAGGTTACCGTGTCCAGC |
| hIL27 | 1395 | CAAGTGCAGCTCCAGGAGTCTGGCGGTGGCTCTGTGCAAGGGGGGGGTTCTCTC |
| Ra_VH | | AGACTCAGCTGCGTGGCCAGCGGCTACGTGTCCTGCGATTACTTCCTGCCTAGC |
| H13- | | TGGTATCGTCAGGCCCCCGGCAAGGAACGCGAGTTCGTCAGCATTATCGACGGG |
| DR595 | | ACTGGGTCAACCTCCTATGCAGCCTCCGTAAAAGGCAGGTTCACCGCCAGTCAG |
| | | GATCGCGGCAAGAATATCGCGTACCTCCAGATGAACTCTTTGAAACCTGAGGAT |
| | | ACAGCTATGTATTACTGTAAGGCTAGTTGTGTGCGTGGGCGCACCATCTCCGAA |
| | | TACTGGGGCCAGGGAACTCAGGTCACTGTTTCTAGTGGGGGGGGAAGTCAGGTG |
| | | CAGTTGCAGGAGTCTGGAGGCGGTTCAGTCCAGGCTGGCGGATCTCTGACGCTC |
| | | TCTTGTGCAGCCTCTGAGTATGCCTATAGCACCTGCAACATGGGATGGTACAGA |
| | | CAGGCTCCTGGTAAGGAGCGCGAATTGGTGAGCGCTTTCATCTCTGATGGTTCC |
| | | ACATATTACGCTGATTCCGTGAAGGGCCGCTTTACGATTACACGCGACAATGCG |
| | | AAAAACACAGTGTACCTCCAGATGAACTCCCTCAAACCAGAAGACACTGCAATC |
| | | TACTATTGTTCTGCTAACTGCTATCGCCGTCTGCGCAACTACTGGGGGCAAGGT |
| | | ACGCAAGTGACCGTCTCCAGC |
| hIL27 | 1396 | CAGGTCCAGCTGCAAGAGAGCGGCGGGCGGTCTGTGCAGGCCGGAGGCAGCCTG |
| Ra_VH | | CGTCTCAGCTGCGTCGCTTCTGGATATGTCAGCTGTGACTACTTCCTCCCATCC |
| H13- | | TGGTACAGACAAGCGCCGGGGAAGGAACGCGAGTTCGTTAGCATTATCGACGGC |
| DR595 | | ACTGGAAGTACCTCTTATGCCGCTTCCGTTAAAGGACGCTTCACGGCCTCTCAG |
| | | GATCGCGGCAAGAATATCGCCTACCTTCAGATGAACAGCCTGAAGCCAGAGGAC |
| | | ACCGCTATGTATTACTGCAAGGCCAGCTGTGTGAGAGGGCGCACCATCAGCGAA |
| | | TACTGGGGCCAGGGCACCCAGGTGACCGTATCTAGTGGGGGGAGCGGTGGCTCC |
| | | GGTGGCAGTGGCCAGGTGCAGCTGCAAGAGAGTGGTGGGGGCAGTGTGCAGGCC |
| | | GGGGGTTCCCTGACGCTGTCCTGTGCGGCCAGCGAGTATGCTTACTCAACGTGG |
| | | AATATGGGTTGGTATCGCCAGGCCCCTGGTAAGGAGCGCGAACTCGTCTCTGCA |
| | | TTTATTAGCGACGGCTCCACTTATTACGCCGACAGCGTGAAGGGGAGATTCACT |
| | | ATTACCCGCGACAATGCCAAGAACACTGTGTACCTCCAAATGAACTCCCTGAAG |
| | | CCCGAGGACACAGCCATCTATTACTGCTCCGCGAACTGCTATCGCCGTCTGCGT |
| | | AACTACTGGGGCCAGGGGACCCAGGTCACAGTCTCCTCT |
| hIL27 | 1397 | CAGGTACAGCTCCAGGAGTCCCGGGGGCGCTCTGTGCAGGCAGGAGGCTCACTG |
| Ra_VH | | CGCTTGAGTTGCGTGGCCAGCGGCTACGTGTCCTGCGACTACTTCCTGCCATCC |
| H13- | | TGGTATAGGCAGGCTCCGGGTAAGGAAAGGGAGTTCGTCTCCATTATCGACGGT |
| DR596 | | ACTGGCAGCACCTCTTACGCAGCCTCCGTGAAGGGGCGTTTCACCGCCAGCCAG |
| | | GATCGGGGCAAGAACATCGCCTACTTGCAGATGAACTCCCTGAAGCCAGAGGAC |
| | | ACTGCAATGTATTACTGCAAAGCCAGCTGTGTGAGAGGACGCACCATCCCTGAG |
| | | TACTGGGGGCAAGGCACACAGGTCACGGTCTCTAGTGGGGGGGGTAGCCAGGTG |
| | | CAGCTCCAGGAAAGCGGAGGTGGCCTGGTTCAGCCGGGAGGGTCACTGAGACTG |
| | | TCCTGTACCGCCAGCGGGCTGACCTTCGATGACAGCGTCATGGGTTGGTTCCGG |
| | | CAGGCCCCCGGTAAAGGAAGAGAGGCGGTGAGGTGCATCAGCTCGTCTGGAGCT |
| | | AACGCCTTCTATGCTGACTCCGTGAAGGGTCGCTTCACCATCTCCAGAGATAAC |
| | | GCTAAGAATACTCTCTACCTCCAGATGAACTCTCTGAAACCGGAGGACACCGCA |
| | | ACCTATTACTGCAAGCGCGGGCACGCCTGCGCGGGATACTATCCAATCCCATAC |
| | | GATGACTACTGGGGTCAGGGAACCCAGGTCACCGTGAGTTCT |
| hIL27 | 1398 | CAGGTGCAGCTCCAGGAGAGCGGCGGTGGCTCTGTCCAGGCAGGCGGTTCCCTG |
| Ra_VH | | CGCCTCTCTTGCGTGGCGAGTGGCTATGTTAGTTGTGACTACTTCCTGCCCTCA |
| H13- | | TGGTATCGGCAAGCTCCGGGAAAAGAGCGCGAGTTTGTCTCCATCATTGATGGA |
| DR596 | | ACAGGATCTACGTCCTATGCCGCAAGTGTCAAGGGGCGGTTCACCGCTTCTCAG |
| | | GACAGAGGAAAGAATATCGCTTATCTCCAGATGAACTCCCTGAAGCCAGAAGAC |
| | | ACCGGTATGTACTATTGCAAGGCTTCTTGTGTGCGCGGCAGAACCATCTCTGAG |
| | | TACTGGGGCCAGGGCACCCAGGTGACCGTTAGTTCAGGAGGGAGCGGAGGCAGT |
| | | GGAGGCTCAGGTCAGGTGCAGCTCCAGGAATCTGGCGGAGGTCTGGTCCAGCCA |
| | | GGTGGGTCCCTGCGCCTCAGCTGTACTGCCTCCGGTCTCACCTTCGATGACAGC |
| | | GTCATGGGCTGGTTCCGCCAAGCGCCGGGTAAGGGGCGTGAAGCCGTGAGCTGC |
| | | ATTTCCTCTAGCGGAGCCAACGCTTTTTACGCCGACAGCGTGAAGGGTAGATTT |
| | | ACAATCTCTCGCGATAACGCGAAGAACACCCTGTATCTCCAAATGAACAGCCTG |
| | | AAGCCAGAGGACACCGCTACTTATTACTGCAAGAGGGGACATGCGTGTGGGGGG |
| | | TATTACCCCATTCCCTATGATGACTACTGGGGCCAGGGCACCCAGGTGACCGTT |
| | | AGTTCT |
| hIL27 | 1399 | CAGGTGCAACTCCAGGAATCCGGCGGTGGCTCCGTGCAAGCCGGTGGCAGTCTT |
| Ra_VH | | AGGCTGTCCTGTGTGGCATCAGGGTATGTGTCCTGCGACTACTTCCTGCCTAGC |
| H14- | | TGGTACAGACAAGCTCCCGGTAAGGAGAGAGAGTTCGTGTCAATCATTGATGGC |
| DR591 | | ACCGGCTCCACTTCCTACGCCGCTTCTGTGAAGGGACGTTTTACTGCCAGCCAA |
| | | GATAAGGGCAAGAACATTGCCTATTTGCAGATGAACTCCTTGAAGCCGGAGGAC |
| | | ACCGCTATGTATTACTGCAAGGCCTCCTGTGTACGTGGGGGGGCCATTTCCGAA |
| | | TACTGGGGCCAAGGAACGCAGGTCACAGTGTCCTCTGGAGGTGGGTCCCAGGTG |
| | | CAGTTGCAGGAAAGCGGCGGAGGGTCCGTCCAGGCTGGTGGCTCTCTGCGCCTT |
| | | TCCTGCACCGCCTCTGGGGCTATCGCCTCCGGGTACATTGATTCTCGCTGGTGC |
| | | ATGGCATGGTTCCGCCAAGCTCCTGGCAAGGAACGCGAGGGAGTGGCCGCGATT |
| | | TGGCCCGGTGGGGGTCTGACTGTCTACGCAGACTCTGTAAAGGGTCGCTTCACA |
| | | ATTTCCAGAGATCACGCGAAGAATACCGTCTACCTCCAGATGAACAATCTCAAG |
| | | CCCGAGGACACTGCCATGTACTATTGCGCCGCTGGATCTCCGCGCATGTGCCCC |
| | | AGTCTGGAATTTGGCTTCGATTACTGGGGCCAGGGTACTCAGGTGACAGTGTCT |
| | | AGC |
| hIL27 | 1400 | CAGGTGCAGTTGCAGGAGTCAGGAGGTGGCAGCGTCCAGGCAGGAGGTAGCTTG |
| Ra_VH | | CGGCTCTCCTGCGTTGCGTCCGGCTATGTGAGTTGTGACTATTTTCTCCCATCT |
| H14- | | TGGTATCGTCAAGCGCCAGGTAAGGAACGTGAATTTGTGTCCATCATTGATGGA |
| DR591 | | ACTGGCTCCACTAGCTACGCCGCGAGCGTGAAGGGCCGGTTCACGGCCTGTCAG |
| | | GATAAGGGGAAGAACATCGCATACCTCCAGATGAACTCCTTGAAGCCAGAGGAT |
| | | ACCGCCATGTATTACTGCAAGGCCTCCTGCGTCCGTGGTCGGGGGATCTCCGAG |
| | | TACTGGGGCCAGGGAACTCAGGTGACAGTCAGGTCTGGTGGCAGGGGAGGGTCC |
| | | GGCGGAAGCGGACAGGTGCAGCTCCAGGAAAGTGGAGGGGGAAGCGTGCAGGCA |
| | | GGGGGCAGCCTGCGGCTGTCTTGTACCGCATCCGGGGCCATTGCTTCCGGCTAT |
| | | ATTGATTCCAGATGGTGCATGGCTTGGTTTAGGCAAGCGCCTGGCAAAGAGCGT |
| | | GAAGGTGTCGCCGCAATCTGGCCGGGGGGGGGTCTGACAGTGTACGCCGACTCT |
| | | GTGAAGGGTAGATTCACAATCAGCCGCGACCATGCTAAAAACACTTTGTACCTC |
| | | CAGATGAATAACCTGAAGCCTGAAGACACCGCCATGTATTACTGCGCTGCCGGA |
| | | AGCCCTCGCATGTGCCCCAGCCTGGAGTTTGGCTTCGATTACTGGGGCCAGGGG |
| | | ACCCAGGTGACGGTGAGCAGC |
| hIL27 | 1401 | CAGGTACAGTTGCAAGAGAGCGGAGGGGGTTCTGTGCAGGCTGGAGGCAGCCTT |
| Ra_VH | | CGCCTGTCCTGCGTGGCCTCTGGATACGTGTCATGTGACTACTTCCTGCCGAGT |
| H14- | | TGGTATCGCCAGGCTCCTGGGAAGGAGCGGGAGTTCGTGAGCATTATCGACGGG |
| DR592 | | ACAGGCAGTACGTCCTACGCGGCCAGCGTGAAAGGTAGGTTCACTGCTTCCCAG |
| | | GATAAAGGCAAAAACATCGCGTACTTGCAGATGAACTCACTGAAGCCTGAGGAC |
| | | ACAGCCATGTATTACTGTAAGGCCAGCTGTGTGCGTGGCCGCGCTATCCCTGAG |
| | | TACTGGGGGCAAGGCACTCAGGTCACTGTCAGCAGTGGTGGAGGTAGCCAGGTG |
| | | CAGCTCCAGGAGTCAGGAGGCGGGTCCGTGCAGGCTGGTGGCAGCCTGCGCCTC |
| | | AGCTGCACCGCTCCCGGCTTCACTTCTAACAGCTGCGGCATGGACTGGTATAGG |
| | | CAAGCCCCAGGGAAGGAGCGCGAGTTCGTTTCCAGTATCTCCACCGATGGAACC |
| | | ACGGGCTATGCTGATTCCGTGAAAGGACGTTTCACTATTAGCAAGGACAAGGCG |
| | | AAAGACACTGTGTATCTCCAGATGAACTCTCTGAAGCCTGAGGATACCGGCATG |
| | | TATAGCTGTAAAACAAAAGATGGGACCATCGCAACTATGGAACTTTGTGACTTC |
| | | GGCTATTGGGGCCAGGGTACTCAGGTAACCGTGTCCTCT |
| hIL27 | 1402 | CAGGTTCAGTTGCAAGAAAGTGGTGGGCGGCAGCGTGCAGGGGGAGGCAGCTTG |
| Ra_VH | | AGGCTGAGCTGCGTGGCATCCGGCTACGTTTCCTGTGATTACTTTCTGCCCTCC |
| H14- | | TGGTATCGCCAAGCGCCTGGAAAAGAGAGGGAGTTCGTTTCTATTATCGACGGC |
| DR592 | | ACAGGCTCCACGAGTTATGCGGCCTCTGTCAAGGGACGCTTTACTGCTTCCCAA |
| | | GACAAGGGGAAGAATATCGCCTATCTCCAGATGAACTCTCTGAAGCCTGAAGAC |
| | | ACCGCTATGTATTACTGCAAGGCCAGCTGTGTGAGAGGTCGCGCTATGTCCGAG |
| | | TACTGGGGACAAGGCACTCAAGTAACGGTGTCATCCGGTGGCTCTGGAGGGTCC |
| | | GGCGGGTCTGGCCAAGTCCAGTTGCAGGAATCTGGTGGAGGGTCTGTCCAGGCA |
| | | GGAGGCAGTCTGCGCCTGTCATGTACCGCGCCCGGATTTACCAGTAATAGCTGC |
| | | GGAATGGATTGGTATCGCCAGGCTCCTGGCAAGGAAAGAGAGTTCGTCTCTTCC |
| | | ATCAGCACTGACGGCACTACAGGCTACGCCGATTCTGTGAAAGGGCGCTTCACA |
| | | ATCTCCAAGGACAAAGCTAAGGATACCGTGTATCTGCAAATGAATAGTCTGAAG |
| | | CCTGAGGATACAGGAATGTACTCCTGTAAGACCAAGGACGGCACCATCGCTACA |
| | | ATGGAGCTGTGCGACTTTGGCTACTGGGGCCAGGGCACCCAAGTGACCGTCTCC |
| | | TCA |
| hIL27 | 1403 | CAGGTGCAGCTCCAGGAGAGTGGTGGAGGGAGCGTGCAGGCTGGAGGCTCCCTG |
| Ra_VH | | CGTCTTAGCTGCGTTGCCAGGGGGTACGTCTCCTGCGACTACTTTCTGCCGTCC |
| H14- | | TGGTATCGTCAAGCCCCTGGTAAAGAAAGAGAGTTCGTCTCCATCATTGATGGA |
| DR593 | | ACAGGCAGCACATCCTATGCGGCCTCCGTGAAGGGCCGCTTTACCGCAAGCCAA |
| | | GATAAAGGTAAGAATATCGCCTACCTCCAGATGAACAGTTTGAAGCCCGAGGAT |
| | | ACAGCCATGTATTACTGTAAGGCCAGTTGTGTGCGGGGGAGAGCCATCAGCGAG |
| | | TACTGGGGCCAGGGCACACAGGTGACTGTATCTTCCGGGGGTGGCTCCCAGGTG |
| | | CAGCTGCAAGAGAGCGGAGGGGGGTCCGTGCAGGCCGGAGGTAGCCTGCGCCTG |
| | | TCATGCGCGGCCTCCGGTTACCCTTACTCCAACGGCTACATGGGGCGGTTTAGA |
| | | CAGGCTCCAGGTAAGGAGCGGGAGGGGGTCGCCACAATCTACACGGGTGATGGC |
| | | AGGACTTACTATGCCGACTCAGTGAAAGGCAGGTTTACCATTAGTCGCGACAAC |
| | | GCCAAGAACACAGTTGACCTTCAGATGTCCTCACTGAAGCCTGAAGACACCGCC |
| | | ATGTATTACTGCGCTGCACGCGCCGCTCCGCTGTACTCCAGCGGCTCCCCATTG |
| | | ACTCGCGCACGCTACAATGTGTGGGGGCAAGGAACCCAGGTGACGGTGTCCTCT |
| hIL27 | 1404 | CAGGTGCAGCTCCAAGAGTCAGGCGGGGGTTCCGTTCAAGCCGGTGGGAGCCTG |
| Ra_VH | | CGCCTGTCATGTGTGGCATCTGGATACGTGTCTTGTGACTACTTCCTGCCATCC |
| H14- | | TGGTATCGGCAGGCCCCTGGGAAGGAGCGCGAATTTGTGTCTATCATTGATGGA |
| DR593 | | ACCGGATCTACAAGCTACGCCGCATCCGTTAAGGGGAGGTTCACAGCCTGCCAG |
| | | GACAAGGGAAAGAACATTGCATACCTCCAGATGAACTCCCTCAAGCCCGAGGAT |
| | | ACTGGTATGTATTACTGTAAGGCCAGTTGTGTGCGTGGACGCGCCATCTCCGAG |
| | | TACTGGGGCCAGGGAACCCAGGTGACCGTGTCCAGCGGTGGCAGCGGCGGTTCC |
| | | GGCGGATCTGGACAGGTGCAGCTCCAGGAGTCTGGAGGCGGGTCCGTTCAGGCT |
| | | GGGGGCAGCTTGCGTTTGAGTTGCGCCGCTTCCGGCTACCCTTACTCTAACGGT |
| | | TACATGGGCTGGTTTCGCCAAGCACCGGGCAAGGAACGGGAAGGTGTCGCTACG |
| | | ATTTACACCGGCGACGGACGCACATATTACGCGGATAGCGTGAAGGGGCGCTTC |
| | | ACAATCTCTAGGGACAACGCGAAGAACACTGTGGACCTCCAGATGTCAAGCCTG |
| | | AAGCCTGAGGATACCGCCATGTACTATTGCGCCGCTCGTGCTGCGCCCCTGTAT |
| | | TCTAGCGGCAGCCCTCTTACTCGGGCACGCTACAACGTGTGGGGTCAAGGCACA |
| | | CAGGTGACCGTCTCCTCC |
| hIL27 | 1405 | CAGGTGCAGGTTCAAGAATCAGGTGGGGGATCTGTGCAGGCAGGGGGGTCACTT |
| Ra_VH | | CGCCTGTCCTGCGTGGCCTCCGGCTATGTCTCTTGTGATTACTTCCTGCCTAGC |
| H14- | | TGGTATCGCCAGGCTCCAGGCAAGGAGAGAGAGTTCGTGAGCATCATTGACGGG |
| DR594 | | ACGGGCTCCACTTCTTACGCCGCAAGCGTCAAGGGGCGGTTCACCGCGTCCCAG |
| | | GACAAGGGAAAGAACATCGCGTACTTGCAGATGAACTCCCTGAAGCCAGAAGAC |
| | | ACAGCTATGTATTACTGTAAGGCGTCTTGCGTCCGTGGCCGTGCAATCTCTGAG |
| | | TACTGGGGCCAGGGCACTCAGGTGACTGTCAGGTCTGGCGGTGGCAGTCAGGTT |
| | | CAGCTTCAGGAATCTGGCGGGGGCTCTGTGCAGGCAGGCGGTAGCCTGAGGTTG |
| | | TCCTGTGTGGCTTCCGCTTCTACCTACTGCACCTACGATATGCACTGGTATCGT |
| | | CAGGCCCCCGGTAAAGGGCGCGAGTTTGTGTCTGCTATTGATTCTGACGGCACC |
| | | ACACGTTATGCCGACTCTGTCAAAGGCAGGTTCACAATCTCTCAAGGTACTGCT |
| | | AAAAACACGGTATACCTTCAAATGAACTCTCTGCAACCTGAAGATACAGCGATG |
| | | TATTACTGTAAGACTGTGTGCGTTGTGGGTAGCAGATGGAGCGACTACTGGGGC |
| | | CAGGGTACACAGGTGACAGTCTCTTCT |
| hIL27 | 1406 | CAGGTCCAGCTTCAGGAGTCTGGGGGGGGTTCTGTGCAGGCTGGGGGCTCTCTC |
| Ra_VH | | CGGCTGAGCTGTGTGGCAAGCGGTTATGTGTCATGCGACTACTTTCTGCCCAGC |
| H14- | | TGGTATCGCCAAGCTCCCGGCAAGGAGAGGGAGTTCGTGTCTATTATCGACGGG |
| DR594 | | ACAGGCTCCACCAGTTACGCGGCCTCAGTGAAGGGACGTTTCACTGCATCTCAA |
| | | GACAAGGGCAAGAACATCGCCTACCTCCAGATGAATAGCCTGAAGCCTGAGGAC |
| | | ACAGCTATGTATTACTGTAAGGCCTCCTGTGTGCGCGGGCGTGCTATCAGCGAA |
| | | TACTGGGGTCAGGGGACTCAAGTCACGGTCTCCTCAGGAGGGTCCGGGGGCAGG |
| | | GGAGGGTCAGGCCAGGTGCAGTTGCAGGAATCTGGAGGCGGTAGTGTTCAGGCC |
| | | GGTGGCTCTGTGCGCCTGTCCTGCGTCGCGAGTGCATCTACGTACTGTACCTAC |
| | | GATATGCACTGGTATCGGCAAGCCCCTGGGAAGGGCCGTGAGTTTGTCAGCGCC |
| | | ATTGATTCTGACGGCACTACCCGCTACGCAGATAGCGTTAAAGGCCGCTTTACT |
| | | ATCAGTCAGGGCACCGCCAAGAACACCGTCTACCTCCAGATGAACTCTCTCCAG |
| | | CCCGAGGATACCGCCATGTACTATTGCAAAACAGTGTGCGTCGTGGGCTCCCGG |
| | | TGGTCCGACTACTGGGGCCAAGGCACTCAAGTGACTGTTTCTTCC |
| hIL27 | 1407 | CAGGTCCAGCTCCAGGAGTCTGGCGGGGGCTCCGTGCAAGCTGGTGGCAGCCTC |
| Ra_VH | | CGTCTGAGCTGCGTAGCCTCTGGTTACGTGTCATGCGACTACTTGCTGCCGTCC |
| H14- | | TGGTATCGGCAGGCCCCCGGCAAAGAACGTGAGTTCGTAAGCATCATTGATGGT |
| DR595 | | ACTGGGAGTACGTCCTATGCAGCCAGCGTGAAGGGCCGCTTTACCGCAAGCCAG |
| | | GACAAGGGTAAAAATATTGGTTATCTTCAGATGAACAGCCTGAAACCTGAAGAT |
| | | ACCGCAATGTATTACTGTAAGGCCAGCTGCGTAAGGGGCCGTGCCATTTCAGAA |
| | | TACTGGGGACAGGGTACACAGGTGACTGTGTCATCTGGGGGGGGTAGCCAGGTG |
| | | CAGCTCCAGGAGTCCGGGGGGGGAGCGTGCAGGCAGGGGCGCTCCCTGACCCTG |
| | | TCCTGTGCGGCCAGCGAGTACGCCTCATAGCACCTGTAATATGGGGGGTATCGT |
| | | CAGGCCCCTGGGAAAGAGCGCGAGCTGGTGTCTGCCTTCATCAGTGACGGGTCC |
| | | ACTTATTACGCCGACTCCGTCAAGGGCCGGTTCACCATTACCCGTGATAACGCC |
| | | AAGAATACCGTTTACCTCCAGATGAATAGCCTCAAACCCGAGGACACCGCGATT |
| | | TACTATTGCTCCGCTAACTGTTACCGCAGGCTGCGTAACTACTGGGGACAGGGC |
| | | ACTCAGGTGACTGTCTCTTCC |
| hIL27 | 1408 | CAGGTCCAGGTCCAGGAGAGCGGCGGGGGTTCCGTCCAGGCCGGTGGCAGCCTG |
| Ra_VH | | CGTCTCTCTTGTGTGGCGAGCGGGTACGTCAGGTGCGATTACTTGCTGCCCTCC |
| H14- | | TGGTATCGCCAGGCCCCTGGTAAGGAGAGGGAGTTCGTGTCTATCATTGATGGG |
| DR595 | | ACTGGATCAACTAGCTACGCTGCGTCTGTTAAAGGGCGGTTTACTGCAAGCCAG |
| | | GACAAGGGCAAGAACATTGCCTACCTTCAGATGAACAGCCTGAAGCCGGAGGAC |
| | | ACCGCCATGTATTACTGTAAGGCCTCTTGTGTACGCGGTCGCGCCATCTCCGAA |
| | | TACTGGGGTCAGGGTACACAGGTGACAGTCAGGTCAGGAGGTTCGGGTGGCTCC |
| | | GGGGGTTCCGGCCAGGTCCAGTTGCAGGAATCCGGTGGCGGAAGCGTGCAGGCT |
| | | GGTGGCTCCCTGACACTGTCTTGCGCAGCCAGCGAATATGCTTATAGTACTTGC |
| | | AACATGGGCTGGTATCGCCAGGCCCCCGGCAAAGAACGCGAACTCGTGAGTGCC |
| | | TTCATCTCCGACGGTAGCACTTATTACGCCGATTCTGTGAAGGGAAGGTTCACT |
| | | ATTACGCGCGACAACGCCAAGAACACCGTCTACTTGCAGATGAACTCCCTGAAG |
| | | CCCGAAGACACTGCCATCTACTATTGCTCCGCCAACTGCTACCGTCGCCTGCGG |
| | | AACTATTGGGGACAGGGCACCCAGGTCACTGTCAGCTCC |
| hIL27 | 1409 | CAAGTGCAGGTGCAAGAGAGTGGAGGTGGCTCCGTACAGGCAGGGGGGAGCCTC |
| Ra_VH | | CGCCTCAGCTGCGTAGCCAGCGGCTATGTCAGTTGCGACTACTTCCTCCCCTCT |
| H14- | | TGGTACAGGCAGGCCCCAGGCAAGGAACGCGAGTTTGTGAGCATTATCGACGGC |
| DR596 | | ACAGGGAGCACGAGTTACGCTGCCAGCGTCAAAGGCAGATTCACAGCCTCTCAA |
| | | GACAAAGGGAAAAACATCGCCTACTTGCAGATGAACTCTCTGAAGCCCGAGGAT |
| | | ACCGCCATGTATTACTGCAAGGCCTCTTGTGTGGGGGGCCGCGCTATCTCCGAA |
| | | TACTGGGGACAGGGGACCCAGGTGACTGTGTCCAGTGGAGGTGGGTCCCAAGTT |
| | | CAGCTCCAGGAGTCCGGTGGCGGGCTTGTGCAGCCGGGCGGTTCCCTGCGCCTG |
| | | TCCTGCACTGCATCTGGTCTGACTTTCGACGATTCCGTAATGGGCTGGTTCCGC |
| | | CAAGCACCTGGCAAAGGTCGCGAGGCTGTATCCTGTATCTCTAGCTCTGGTGCC |
| | | AACGCATTCTACGCTGACTCCGTCAAAGGCCGTTTTACCATTAGTCGCGATAAC |
| | | GCTAAAAACACACTGTACCTGCAAATGAACTCCCTCAAACCGGAAGATACTGCC |
| | | ACCTACTATTGCAAGCGCGGGCACGCCTGTGGGGGCTATTACCCTATCCCATAC |
| | | GATGACTACTGGGGCCAGGGCACCCAGGTCACTGTTAGCTCC |
| hIL27 | 1410 | CAGGTCCAACTCCAGGAGTCCGGCGGGGGCAGTGTGCAGGCTGGGGGGTCTCTG |
| Ra_VH | | CGTCTGTCCTGCGTCGCGAGCGGCTACGTCAGGTGCGACTATTTTCTGCCTAGC |
| H14- | | TGGTATCGCCAGGCCCCAGGTAAGGAAAGAGAGTTCGTGAGCATTATCGACGGG |
| DR596 | | ACAGGTTCCACCTCCTACGCCGCGTCTGTGAAGGGCCGGTTTACTGCTTCCCAG |
| | | GATAAAGGTAAGAACATTGCATATCTGCAAATGAACTCTCTGAAGCCCGAGGAC |
| | | ACTGCTATGTATTACTGTAAAGCAAGTTGTGTGCGCGGCAGAGCAATTTCTGAA |
| | | TACTGGGGCCAGGGCACCCAGGTGACTGTGTCCAGCGGGGGCAGTGGTGGCAGT |
| | | GGAGGTAGCGGCCAAGTCCAGTTGCAGGAGAGTGGGGGTGGCCTGGTGCAGCCA |
| | | GGTGGCTCCCTCCGTCTGAGCTGCACAGCCAGCGGGCTGACGTTCGATGACTCT |
| | | GTGATGGGTTGGTTTCGCCAGGCTCCCGGCAAGGGCCGCGAAGCTGTTTCCTGT |
| | | ATCTCTTCCTCTGGCGCGAATGCTTTTTACGCCGACTCCGTGAAGGGGAGGTTC |
| | | ACTATTTCCAGAGACAATGCCAAGAACACTCTGTACCTTCAGATGAACTCCCTC |
| | | AAGCCTGAAGACACCGCCACCTACTATTGTAAGCGCGGCCATGCGTGTGCCGGG |
| | | TACTATCCTATTCCTTATGATGACTACTGGGGCCAGGGCACTCAGGTGACCGTG |
| | | TCCTCC |
| hIL27 | 1411 | CAGGTGCAGCTCCAGGAGAGCGGCGGTGGCTCCGTGCAGGCCGGTGGCTCCCTC |
| Ra_VH | | CGCCTCAGCTGCGTGGCCTCAGGTTACGTCAGCTGCGATTACTTTCTGCCAAGC |
| H15- | | TGGTATCGCCAAGCGCCGGGGAAGGAGCGCGAGTTTGTGTCTATTATCGACGGA |
| DR591 | | ACTGGCTCCACTAGCTATGCTGCCAGCGTGAAGGGACGCTTCACAGCTTCCCAG |
| | | GATAAGGGAAAAAACATCGCCTACCTCCAGATGAACACCCTGAAACCCGAGGAC |
| | | ACCGCCATGTATTACTGCAAGGCAAGCTGCGTGCGCGGCAGAGCTATCCCCGAG |
| | | TACTGGGGCCAGGGAACCCAGGTTACCGTCAGCTCAGGCGGAGGGTCACAGGTT |
| | | CAGTTGCAAGAAAGCGGCGGTGGGAGCGTGCAGGCTGGTGGAAGCCTGCGTCTG |
| | | TCCTGCACAGCATCCGGCGCAATCGCTAGTGGATACATTGATAGCCGCTGGTGC |
| | | ATGGCCTGGTTCCGCCAAGCTCCAGGTAAAGAGCGCGAGCGGGGGCGGGCCATG |
| | | TGGCCTGGCGGAGGCCTGACTGTTTACGCCGACTCAGTGAAGGGGCGCTTTACC |
| | | ATCTCACGGGACCATGCTAAGAACACCCTGTACTTGCAGATGAACAATCTGAAA |
| | | CCTGAGGACACCGCGATGTATTACTGCGCAGCGGGCTCACCTCGTATGTGCCCT |
| | | AGCTTGGAGTTTGGCTTCGACTACTGGGGCCAAGGCACCCAGGTGACTGTGAGC |
| | | AGC |
| hIL27 | 1412 | CAAGTGCAGTTGCAGGAGAGTGGTGGGGGCTCTGTGCAGGCCGGGGGTTCCCTG |
| Ra_VH | | AGGCTCTCTTGCGTGGCCAGGGGATACGTGTCCTGTGACTACTTCCTGCCCTCC |
| H15- | | TGGTATCGCCAAGCGCCAGGGAAGGAACGCGAGTTCGTGAGCATTATCGACGGC |
| DR591 | | ACTGGTTCCACCTCTTACGCAGCGTCTGTAAAGGGCCGTTTCACCGCCAGCCAG |
| | | GACAAGGGGAAGAACATCGCTTACCTTCAGATGAATACGTTGAAGCCTGAGGAC |
| | | ACTGCGATGTATTACTGTAAAGCGTCTTGCGTTCGTGGCCGTGCCATCTCCGAA |
| | | TACTGGGGCCAAGGCACTCAGGTAACTGTGTCCAGCGGCGGTTCCGGTGGCTCT |
| | | GGAGGTAGCGGTCAGGTGCAGCTCCAGGAATCTGGCGGTGGGAGTGTGCAGGCT |
| | | GGCGGTAGTCTCCGGCTGTCCTGTACCGCCAGTGGAGCTATTGCCAGCGGATAC |
| | | ATTGATTCCCGGTGGTGTATGGCATGGTTCCGGCAGGCCCCCGGCAAGGAACGC |
| | | GAGGGGGTCGCCGCTATTTGGCCCGGAGGCGGGCTGACCGTGTACGCGGACAGT |
| | | GTCAAGGGCCGCTTCACGATCAGTAGGGATCACGCCAAGAACACCCTCTACTTG |
| | | CAGATGAATAACCTCAAACCAGAGGACACTGCAATGTATTACTGCGCCGGTGGC |
| | | AGCCCGAGGATGTGCCCGTCCCTGGAGTTCGGCTTCGACTATTGGGGTCAGGGC |
| | | ACTCAGGTCACCGTGTCCTCC |
| hIL27 | 1413 | CAGGTCCAGGTCCAGGAGAGCGGCGGAGGCTCTGTTCAGGCTGGGGGCAGCCTC |
| Ra_VH | | CGCCTGTCTTGCGTGGCCTCCGGCTACGTAAGGTGCGACTACTTGTTGCCTTCC |
| H15- | | TGGTATCGCCAGGCTCCGGGCAAAGAAAGGGAATTTGTATCCATTATCGACGGC |
| DR592 | | ACGGGTAGCACTTCCTACGCCGCTAGTGTAAAGGGTCGGTTCACCGCCTCTCAG |
| | | GACAAGGGCAAAAACATCGCGTACCTCCAGATGAACACACTGAAGCCAGAGGAT |
| | | ACCGCCATGTATTACTGCAAGGCTTCCTGCGTGCGGGGGGGGGCCATTAGCGAG |
| | | TACTGGGGACAAGGAACCCAAGTGACTGTGTCCAGCGGTGGAGGCTCTCAGGTC |
| | | CAGCTGCAAGAGTCTGGAGGCGGTTCAGTGCAGGCCGGTGGAAGCCTTAGGCTG |
| | | AGCTGCACCGCCCCTGGATTTACCTCTAACTCTTGCGGCATGGACTGGTATCGT |
| | | CAGGCCCCCGGCAAGGAACGCGAGTTCGTGTCTAGCATCTCCACCGATGGCACC |
| | | ACAGGCTATGCTGACAGTGTGAAAGGCCGTTTTACCATCTCCAAGGACAAAGCC |
| | | AAGGACACGGTGTACCTCCAGATGAACTCCCTGAAGCCTGAAGACACGGGTATG |
| | | TATTCTTGCAAGACTAAGGACGGCACCATCGCTACTATGGAGCTGTGCGACTTC |
| | | GGTTATTGGGGACAGGGGACCCAGGTGACTGTATCTTCT |
| hIL27 | 1414 | CAGGTGCAGCTGCAAGAGTCTGGAGGTGGGTCAGTCCAGGCTGGGGGCAGCCTC |
| Ra_VH | | AGGCTGTCCTGCGTCGCTTCTGGATACGTGTCGTGCGACTACTTGCTCCCCTCC |
| H15- | | TGGTATCGCCAGGCCCCAGGTAAAGAACGCGAGTTCGTGTCCATCATTGATGGC |
| DR592 | | ACCGGGTCTACTTCCTATGCTGCGTCCGTGAAGGGCCGTTTTACCGCTTCACAG |
| | | GATAAAGGAAAGAACATTGCTTACCTCCAGATGAACACACTCAAGCCGGAAGAC |
| | | ACCGCCATGTATTACTGTAAAGCGAGCTGTGTCCGTGGCAGAGCCATCAGTGAG |
| | | TATTGGGGCCAGGGAACTCAGGTGACCGTGTCCTCTGGAGGCAGCGGCGGTAGT |
| | | GGTGGCTCCGGGCAAGTTCAACTTCAGGAGAGTGGGCGGGGGTCCGTGCAGGCT |
| | | GGGGGTTCTCTCCGCCTCAGCTGTACCGCGCCCGGCTTCACCTCTAACAGCTGC |
| | | GGTATGGACTGGTATCGCCAAGCGCCGGGTAAAGAGCGTGAGTTCGTTAGCTCC |
| | | ATCTCCACCGACGGCACCACAGGATATGCCGATAGCGTTAAGGGACGCTTTACC |
| | | ATTAGTAAGGACAAGGCTAAGGATACCGTCTACCTCCAGATGAACTCTCTGAAA |
| | | CCTGAAGACACAGGCATGTACTCCTGTAAAACCAAGGACGGCACCATTGCTACA |
| | | ATGGAGCTTTGTGATTTCGGCTATTGGGGCCAGGGTACACAGGTTACCGTGTCC |
| | | TCC |
| hIL27 | 1415 | CAAGTGCAGTTGCAGGAGTCCGGCGGAGGTTCAGTTCAGGCTGGCGGGTCCCTG |
| Ra_VH | | AGACTGAGTTGTGTGGCCTCCGGCTATGTGAGCTGCGACTACTTTCTGCCTTCT |
| H15- | | TGGTACAGACAAGCACCAGGCAAAGAGGGGGAGTTCGTGAGTATCATTGATGGG |
| DR593 | | ACCGGCTCCACCAGCTATGCTGCCAGCGTGAAGGGTCGCTTTACCGCTTCTCAG |
| | | GACAAAGGCAAAAACATCGCTTACCTCCAGATGAATACATTGAAGCCTGAAGAC |
| | | ACCGCCATGTATTACTGTAAGGCATCATGTGTGCGTGGCCGCGCCATTTCTGAG |
| | | TACTGGGGCCAGGGAACTCAGGTGACAGTGTCTAGTGGCGGAGGCTCCCAGGTC |
| | | CAGTTGCAGGAGAGCGGCGGTGGATCTGTCCAGGGGGGCGGCAGCCTCCGTCTG |
| | | TCCTGTGCGGCCAGGGGGTATCCTTATTCCAACGGCGTACATGGGGGGTTCCGC |
| | | CAGGCCCCCGGTAAGGAACGCGAGGGCGTGGCCACTATTTACACAGGTGACGGG |
| | | CGTACCTATTACGCAGACTCTGTAAAGGGTCGCTTCACAATTTCCCGCGACAAC |
| | | GCCAAGAACACCGTCGATCTCCAGATGTCTAGTCTCAAACCTGAGGACACCGCC |
| | | ATGTATTACTGTGCCGCTCGCGCCGCGCCACTTTACAGCTCTGGTTCTCCCCTC |
| | | ACACGCGCTCGCTACAATGTTTGGGGCCAAGGCACACAGGTGACCGTGAGCAGT |
| hIL27 | 1416 | CAGGTGCAGTTGCAGGAATCTGGTGGAGGCTCCGTGCAGGCTGGGGGCTCCCTG |
| Ra_VH | | AGGCTTTCATGTGTGGCAAGCGGCTACGTGAGTTGTGATTACTTTCTGCCTTCC |
| H15- | | TGGTATCGCCAGGCCCCTGGCAAGGAGCGGGAATTTGTCTCAATCATTGATGGT |
| DR593 | | ACTGGTTCCACTTCTTATGCGGCCAGTGTGAAAGGGCGTTTTACTGCCAGCCAG |
| | | GATAAGGGCAAGAATATTGCTTACTTGCAGATGAACACCCTGAAGCCCGAAGAT |
| | | ACCGCTATGTACTATTGCAAGGCCAGTTGTGTAAGGGGTCGGGCGATTAGTGAG |
| | | TATTGGGGCCAGGGCACGCAGGTCACCGTGTCATCCGGGGGCAGCGGGGGGTCC |
| | | GGGGGTTCTGGCCAGGTGCAGCTCCAGGAGAGCGGTGGAGGCAGCGTGCAGGCC |
| | | GGTGGAAGCCTTCGTCTGTCCTGCGCCGCTTCAGGCTATCCTTACTCTAATGGC |
| | | TACATGGGTTGGTTTCGCCAAGCCCCAGGGAAAGAGCGCGAGGGCGTTGCCACA |
| | | ATTTACACAGGTGATGGAAGGACCTACTATGCGGACAGCGTCAAGGGCCGCTTT |
| | | ACAATCAGCCGTGATAATGCTAAGAACACCGTCGATCTCCAGATGTCCAGCCTG |
| | | AAGCCTGAGGATACTGCTATGTACTATTGCGCTGCCCGCGCAGCCCCACTCTAC |
| | | AGCTCCGGCAGTCCCCTGACGAGGGCTCGCTATAACGTGTGGGGTCAGGGCACA |
| | | CAGGTCACCGTGTCCAGC |
| hIL27 | 1417 | CAGGTGCAACTCCAGGAATCCGGGGGAGGCAGCGTGCAAGCCGGTGGCTCCCTG |
| Ra_VH | | CGCCTGTCCTGCGTCGCCAGCGGCTACGTCAGCTGCGACTACTTTCTGCCCTCC |
| H15- | | TGGTATCGCCAGGCTCCTGGGAAAGAGCGCGAGTTTGTTTCCATCATTGATGGA |
| DR594 | | ACCGGCAGCACATCCTATGCCGCGAGCGTTAAGGGCAGATTTACCGCCTCTCAA |
| | | GACAAGGGTAAGAACATCGCCTACCTCCAAATGAACACCCTGAAGCCTGAGGAC |
| | | ACGGCAATGTATTACTGTAAAGCAAGCTGTGTGCGGGGTCGGGCCATCAGCGAG |
| | | TACTGGGGTCAGGGCACCCAGGTCACTGTAAGCTCCGGGGGAGGGTCCCAGGTG |
| | | CAGCTCCAGGAGTCAGGCGGAGGCTCCGTGCAGGCCGGAGGCCCCTTGCGTTTG |
| | | AGCTGCGTGGCGTCTGCGTCTACCTACTGCACTTACGACATGCACTGGTATCGC |
| | | CAGGCTCCGGGGAAGGGCCGCGAGTTCGTGAGCGCTATCGACTCCGATGGCACA |
| | | ACTCGCTACGCCGACAGCGTGAAGGGGCGTTTTACCATTTCCCAGGGCACCGCC |
| | | AAGAACACCGTGTATGTCCAGATGAACTCTCTGCAACCGGAGGATACAGCTATG |
| | | TATTACTGCAAGACAGTCTGTGTGGTTGGTAGCCGCTGGTCTGACTACTGGGGC |
| | | CAGGGCACCCAGGTCACGGTGTCCTCT |
| hIL27 | 1418 | CAGGTGCAGCTTCAAGAGTCTGGTGGCGGGTCCGTCCAGGCGGGTGGCAGTTTG |
| Ra_VH | | AGACTCAGTTGTGTGGCCAGTGGCTACGTCAGCTGTGACTATTTTCTGCCTAGG |
| H15- | | TGGTATCGCCAGGCACCCGGTAAAGAGAGGGAGTTCGTGAGCATCATTGATGGG |
| DR594 | | ACAGGGTCTACATCTTATGCTGCCTCCGTGAAAGGGGGGTTCACAGCTTCCCAG |
| | | GATAAGGGCAAGAACATTGCGTATCTCCAGATGAACACCCTCAAGCCGGAGGAT |
| | | ACCGCGATGTATTACTGTAAGGCGTCTTGTGTGCGCGGGCGTGCTATTAGCGAG |
| | | TATTGGGGACAGGGGACGCAAGTGACGGTCTCCTCAGGCGGGTCTGGTGGCTCA |
| | | GGCGGGTCCGGGCAGGTCCAGCTCCAGGAGTCTGGCGGTGGCTCCGTCCAAGCC |
| | | GGAGGCAGCCTCCGTCTCTCCTGTGTTGCATCAGCCTCCACGTACTGCACCTAT |
| | | GATATGCACTGGTATCGGCAAGCGCCCGGCAAGGGCCGCGAGTTTGTGTCTGCC |
| | | ATCGACAGTGATGGTACAACGCGGTACGCTGATAGCGTGAAAGGCCGGTTCACC |
| | | ATCAGTCAGGGCACGGCCAAAAACACAGTGTATCTCCAGATGAACTCTCTCCAG |
| | | CCTGAAGATACAGCCATGTACTATTGTAAGACGGTCTGCGTCGTGGGCAGCCGG |
| | | TGGTCCGACTACTGGGGCCAGGGAACCCAAGTGACTGTCTCAAGT |
| hIL27 | 1419 | CAGGTGCAGTTGCAGGAGAGCGGCGGAGGCTCTGTGCAGGCAGGAGGCTCACTG |
| Ra_VH | | AGACTGTCCTGCGTGGCAAGGGGCTATGTGAGGTGCGACTACTTTCTGCCAAGT |
| H15- | | TGGTATCGTCAGGCTCCTGGCAAAGAAAGGGAGTTTGTGAGCATCATTGATGGA |
| DR595 | | ACCGGAAGCACAAGTTATGCGGCCTCCGTGAAGGGTCGCTTCACCGCCTCACAG |
| | | GATAAGGGCAAGAACATTGCTTACCTCCAGATGAACACACTCAAGCCTGAAGAC |
| | | ACCGCGATGTACTATTGTAAAGCAAGTTGTGTGAGAGGCCGCGCCATTAGCGAG |
| | | TATTGGGGCCAGGGCACTCAGGTGACCGTCAGCTCTGGGGGGGGAAGCCAGGTA |
| | | CAACTCCAGGAGAGTGGTGGAGGCTCAGTGCAGGCCGGGGGAAGCCTGACCCTG |
| | | TCCTGTGCCGCTTCCGAGTACGCATACAGCACTTGCAACATGGGCTGGTATCGC |
| | | CAAGCCCCTGGCAAAGAACGGGAACTCGTGTCCGCCTTCATCTCCGATGGTTCT |
| | | ACCTATTACGCTGATAGCGTGAAAGGACGCTTCACCATTACCCGTGATAACGCT |
| | | AAGAACACAGTTTACTTGCAGATGAACTCTCTGAAGCCCGAAGACACCGCCATT |
| | | TATTACTGTAGCGCCAACTGTTATCGCAGGCTTCGGAATTACTGGGGACAGGGG |
| | | ACCCAGGTGACAGTGAGTAGC |
| hIL27 | 1420 | CAGGTGCAGTTGCAGGAGTCTGGGGGGGGTAGCGTCCAGGCTGGGGGATCACTG |
| Ra_VH | | CGCCTGAGTTGTGTCGCGTCCGGCTACGTCAGCTGCGACTATTTCTTGCCCTCC |
| H15- | | TGGTATCGCCAGGCCCCTGGCAAGGAACGTGAGTTCGTAAGCATCATTGACGGC |
| DR595 | | ACTGGTAGCACTTCCTACGCCGCTAGTGTGAAGGGGCGGTTCACCGCAAGCCAG |
| | | GACAAGGGGAAGAACATCGCCTACCTCCAGATGAACACACTGAAGCCCGAGGAC |
| | | ACGGCCATGTACTATTGCAAAGCCTCTTGCGTGCGGGGGCGTGCGATCTCTGAA |
| | | TATTGGGGCCAGGGAACTCAGGTGACCGTGTCTAGTGGTGGCAGTGGCGGGTCC |
| | | GGGGGGAGCGGTCAGGTTCAGTTGCAGGAGAGCGGCGGTGGCTCCGTTCAGGCC |
| | | GGGGGCTCTCTGACGCTGTCTTGCGCGGCCAGCGAATACGCTTACTCAACTTGT |
| | | AATATGGGTTGGTATCGTCAGGCACCCGGCAAGGAGCGGGAGCTGGTCTCCGCT |
| | | TTCATCAGCGACGGCTCCACCTATTACGCAGACAGCGTTAAGGGTCGCTTTACT |
| | | ATCACGCGCGACAACGCCAAGAACACCGTGTACCTCCAGATGAACTCTCTGAAA |
| | | CCCGAGGATACCGCCATTTACTATTGCTCTGCTAACTGGTACCGCAGGTTGAGA |
| | | AATTACTGGGGCCAAGGAACTCAGGTGACGGTCTCAAGC |
| hIL27 | 1421 | CAGGTGCAGCTCCAGGAATCTGGCGGTGGCTCCGTACAGGCTGGCGGTTCCCTG |
| Ra_VH | | AGGCTCTCTTGTGTCGCCAGCGGCTACGTCAGCTGTGACTATTTCCTCCCTTCT |
| H15- | | TGGTATCGTCAGGCCCCAGGGAAGGAGCGCGAGTTCGTGTCCATTATGGACGGG |
| DR596 | | ACCGGAAGCACCTCCTATGCTGCATCAGTGAAAGGGCGGTTTACCGCGTCTCAG |
| | | GACAAAGGAAAGAACATTGCATATCTGCAAATGAACACACTCAAGCCAGAGGAC |
| | | ACCGCTATGTACTATTGTAAGGCCTCCTGTGTGAGAGGTCGCGCAATTTCTGAA |
| | | TATTGGGGCCAGGGAACCCAGGTGACCGTGTCCTCTGGGGGGGGTTCTCAGGTC |
| | | CAACTTCAGGAAAGCGGGGCGGGGCTCGTGCAACCCGGTGGCAGCCTTCGGCTG |
| | | TCTTGTACTGCCTCCGGTTTGACATTCGATGACTCTGTGATGGGATGGTTTAGG |
| | | CAGGCTCCTGGCAAAGGCCGCGAGGCAGTATCTTGTATCAGTTCTTCCGGCGCT |
| | | AACGCATTTTATGCCGACAGTGTGAAGGGCCGTTTCACAATCTCCCGCGATAAC |
| | | GCCAAGAACACCCTCTACCTCCAGATGAACTCCCTGAAGCCTGAGGACACCGCC |
| | | ACTTATTACTGCAAGCGGGGTCATGCCTGTGCTGGCTATTACCCTATTCCATAC |
| | | GATGACTACTGGGGACAGGGCACCCAAGTTACCGTGTCTAGC |
| hIL27 | 1422 | CAGGTGCAGCTTCAGGAGTCAGGAGGTGGCTCAGTGCAGGCTGGTGGCAGTCTG |
| Ra_VH | | CGCCTGTCCTGTGTCGCCAGTGGATATGTCAGTTGTGACTATTTTCTCCCCAGT |
| H15- | | TGGTATCGCCAAGCGCCCGGTAAAGAGCGGGAGTTTGTCTCAATCATTGACGGC |
| DR596 | | ACCGGCAGCACCTCCTACGCCGCTTCCGTGAAGGGTCGCTTCACCGCGTCCCAG |
| | | GACAAAGGCAAGAACATCGCGTATCTTCAGATGAACACCCTCAAGCCCGAAGAT |
| | | ACAGCTATGTATTACTGCAAGGCCTCCTGCGTGCGGGGACGCGCGATCTCTGAA |
| | | TACTGGGGCCAGGGCACACAGGTCACCGTCTCTTCAGGGGGTTCCGGTGGCTCC |
| | | GGTGGCTCCGGCCAGGTGCAGTTGCAGGAAAGCGGTGGCGGGCTGGTGCAGCCG |
| | | GGGGGGAGTCTCCGCCTGTCCTGCACAGCGTCAGGGCTGACTTTTGATGACTCT |
| | | GTTATGGGCTGGTTTAGGCAGGCTCCGGGTAAGGGCAGAGAGGCCGTTAGCTGC |
| | | ATCTGTTCCAGCGGGGCTAACGCCTTCTATGCCGACAGCGTGAAGGGTCGCTTC |
| | | ACAATTAGCCGGGACAACGCTAAGAACACTCTGTACCTTCAGATGAACTCCCTG |
| | | AAGCCCGAAGATACTGCTACTTACTATTGCAAGAGAGGTCATGCTTGCGCCGGA |
| | | TACTATCCAATCCCTTACGATGACTACTGGGGCCAAGGCACCCAAGTTACCGTG |
| | | TCTTCT |
| hIL27 | 1423 | CAGGTGCAGTTGCAGGAAAGTGGCGGTGGATCTGTGCAGGCAGGGGGAAGTCTG |
| Ra_VH | | AGACTGAGCTGCCGGGCCAGCGGTTCAACTTATAGCAATTACTGTCTGGGTTGG |
| H16- | | TTCAGGCAGATTACTGGTAAGGAACGCGAAGGCGTTGCTGTTATCAACTGGGTT |
| DR591- | | GGGGGAATGCTGTACTTTGCTGACTCCGTTAAGGGCAGATTCACTGTCAGCCAG |
| | | GACCAGGCCAAGAATACCGTGTACTTGCAGATGAACAGCCTGAAGCCCGAGGAT |
| | | ACAGCCATGTATTACTGTGCTGCCGAGTCAGCCTCCTCTTTCTCATGTGGGGGA |
| | | TGGCTTACAAGGCCCGACCGCGTTCCATATTGGGGCCAGGGCACCCAGGTGACT |
| | | GTAAGCTCCGGTGGAGGCAGTCAGGTCCAGCTCCAGGAGAGCGGCGGAGGCTCT |
| | | GTTCAGGCTGGAGGTAGCCTGCGCCTTAGCTGCACAGCGTCAGGTGCCATCGCC |
| | | AGCGGTTATATTGATTCACGCTGGTGTATGGCCTGGTTTCGCCAGGCCCCTGGG |
| | | AAGGAGAGGGAAGGTGTGGCTGCCATTTGGCCCGGAGGCGGTCTGACAGTCTAC |
| | | GCAGACTCCGTGAAGGGCCGGTTTACCATCAGCCGGGACCACGCGAAGAACACT |
| | | CTGTATTTGCAAATGAATAACCTCAAACCCGAGGACACCGCAATGTACTATTGT |
| | | GCCGCTGGCTCTCCTCGTATGTGTCCATCTCTGGAGTTCGGCTTTGACTACTGG |
| | | GGCCAGGGGACCCAGGTGACTGTGAGTAGC |
| hIL27 | 1424 | CAAGTGCAGTTGCAGGAGTCTGGTGGCGGTTCCGTGCAGGCTGGTGGAAGCCTG |
| Ra_VH | | AGGCTGTCTTGTCGTGCATCTGGAAGCACCTATTCCAACTATTGTCTTGGGTGG |
| H16- | | TTCAGACAGATCACTGGCAAAGAGAGAGAAGGGGTTGCGGTTATCAACTGGGTG |
| DR591 | | GGCGGTATGGTGTACTTCGCTGATTCCGTGAAGGGCCGCTTCACGGTATCCCAG |
| | | GACCAAGCAAAGAATACCGTCTATTTGCAGATGAACAGCCTGAAACCGGAGGAT |
| | | ACCGCCATGTACTATTGTGCAGCCGAGAGCGCCAGCTCCTTCTCTTGTGGCGGT |
| | | TGGCTGACTCGTCCAGATCGCGTGCCCTACTGGGGCCAAGGGACCCAGGTGACA |
| | | GTCAGCTCCGGCGGTAGCGGTGGCTCTGGGGGCTCCGGGCAGGTGCAGCTCCAG |
| | | GAAAGTGGGGGGGGTTCAGTGCAGGCAGGTGGCAGTCTGCGTCTGTCCTGCACC |
| | | GCAAGCGGCGCAATCGCTTCCGGTTATATTGATTCTCGCTGGTGCATGGCTTGG |
| | | TTTCGCCAGGCCCCCGGCAAGGAACGCGAGGGGGTGGCAGCCATCTGGCCGGGA |
| | | GGGGGACTGACCGTTTACGCTGACAGCGTGAAGGGGCGCTTCACGATCAGTAGG |
| | | GATCACGCGAAGAACACCCTTTACCTCCAGATGAATAACCTGAAGCCAGAGGAC |
| | | ACAGCGATGTATTACTGCGCCGCTGGTTCTCCCCGGATGTGTCCATCCCTGGAG |
| | | TTCGGGTTCGATTACTGGGGCCAGGGGACGCAAGTGACAGTTAGCTCC |
| hIL27 | 1425 | CAGGTACAGCTGCAAGAAAGCGGCGGAGGGTCCGTGCAGGCTGGCGGTTCTCTT |
| Ra_VH | | CGTCTGTCTTGCCGCGCCTCCGGTAGTACCTACTCCAACTACTGCTTGGGCTGG |
| H16- | | TTCCGCCAGATCACAGGCAAGGAGCGTGAGGGGGTGGCCGTAATAAATTGGGTG |
| DR592 | | GGCGGTATGGTGTATTTCGCAGATTCCGTGAAAGGCCGCTTTACCGTCTCCCAG |
| | | GACCAGGCCAAAAACACCGTGTACCTCCAGATGAACTCTCTGAAGCCGGAGGAC |
| | | ACAGCGATGTACTATTGTGCTGCCGAAAGCGCTTCTAGTTTTAGCTGTGGCGGT |
| | | TGGCTGACCCGTCCTGATCGCGTTCCATACTGGGGACAGGGTACTCAGGTCACC |
| | | GTGTCTTCCGGCGGTGGCTCCCAGGTTCAGCTCCAGGAATCTGGCGGTGGGAGT |
| | | GTCCAGGCTGGAGGTAGTCTGCGCCTGTCATGTACCGCCCCCGGTTTTACCTCA |
| | | AACTCTTGCGGTATGGACTGGTATCGCCAGGCTCCCGGTAAGGAGAGGGAGTTC |
| | | GTCAGCTCTATCAGTACTGACGGCACTACAGGATACGCCGACTCTGTGAAGGGC |
| | | CGTTTCACCATCTCTAAGGACAAGGCTAAAGACACCGTCTACCTCCAGATGAAC |
| | | AGTCTCAAGCCCGAGGACACGGGTATGTATTCCTGCAAAACAAAGGATGGGACT |
| | | ATTGCCACAATGGAGCTTTGTGACTTTGGCTACTGGGGACAGGGAACACAGGTG |
| | | ACTGTCTCAAGC |
| hIL27 | 1426 | CAGGTGCAGTTGCAGGAGTCAGGAGGTGGGTCCGTGCAGGCCGGTGGCAGCCTG |
| Ra_VH | | CGGCTCTCTTGTCGGGCCAGTGGCAGCACTTATAGTAACTACTGTCTGGGGTGG |
| H16- | | TTCCGCCAGATCACTGGTAAAGAGCGGGAGGGCGGGGCTGTCATTAACTGGGTC |
| DR592 | | GGCGGTATGCTGTACTTCGCCGATTCCGTGAAGGGCCGTTTCACAGTGTCCCAA |
| | | GACCAGGCCAAGAACACAGTTTACCTTCAGATGAACAGCCTGAAGCCAGAGGAC |
| | | ACCGCCATGTACTATTGCGCCGCAGAATCTGCTTCTAGCTTCAGCTGCGGCGGT |
| | | TGGCTGACACGCCCGGACCGCGTGCCGTACTGGGGGCAGGGCACACAGGTAACC |
| | | GTGTCCTCTGGCGGGAGTGGGGGGTCCGGGGGTAGCGGCCAAGTCCAGCTCCAG |
| | | GAGTCAGGCGGAGGCTCCGTCCAGGCTGGTGGCAGCTTGAGGTTGTCCTGCACT |
| | | GCTCCTGGTTTTACATCTAACTCCTGGGGCATGGATTGGTATCGCCAGGGTCCC |
| | | GGAAAGGAGCGCGAGTTCGTGTCCTCTATTAGTACCGATGGCACTACCGGGTAT |
| | | GCCGACTCTGTTAAGGGTCGTTTCACAATCAGTAAAGATAAGGCCAAGGACACA |
| | | GTGTACTTGCAGATGAACTCACTGAAACCAGAGGATACAGGCATGTATAGCTGT |
| | | AAGACCAAAGACGGGACCATCGCAACTATGGAGCTGTGCGATTTGGGCTACTGG |
| | | GGCCAGGGCACCCAGGTAACCGTCAGCTCA |
| hIL27 | 1427 | CAGGTACAGTTGCAGGAGTCCGGCGGGGGAAGCGTGCAGGCTGGAGGTAGCCTG |
| Ra_VH | | CGTCTGAGCTGTCGCGCCAGCGGCTCCACCTACTCAAACTACTGTCTTGGATGG |
| H16- | | TTCCGTCAGATTACTGGCAAGGAGAGGGAAGGGGTCGCCGTTATCAACTGGGTG |
| DR593 | | GGCGGTATGCTGTACTTCGCCGACTCTGTTAAAGGCCGTTTCACAGTGAGCCAG |
| | | GATCAGGCCAAGAACACCGTCTACCTTCAGATGAACTCTTTGAAGCCTGAAGAT |
| | | ACGGCCATGTATTACTGTGCAGCCGAGTCTGCGTCCAGCTTCTCGTGCGGGGGC |
| | | TGGCTCACACGCCCCGACCGTGTGCCTTATTGGGGACAGGGGACACAAGTCACT |
| | | GTCTCCTCTGGAGGGGGATCTCAAGTGCAGCTGCAAGAGTCTGGAGGGGGTAGC |
| | | GTGCAGGCTGGGGGGTCCCTCAGACTGAGCTGTGCTGCCTCCGGTTACCCCTAC |
| | | TCCAACGGATATATGGGCTGGTTCCGCCAGGCACCAGGCAAGGAGCGCGAGGGC |
| | | GTGGCTACTATCTATACAGGTGATGGCCGCACCTATTACGCTGATTCCGTGAAG |
| | | GGTCGCTTCACCATCAGCCGCGACAATGCTAAGAACACCGTGGACCTTCAGATG |
| | | TCTAGCCTCAAGCCTGAGGACACTGCAATGTATTACTGTGCTGCACGGGCAGCG |
| | | CCCCTTTATTCCTCTGGCTCCCCTCTCACCCGCGCACGTTACAACGTATGGGGT |
| | | CAAGGCACCCAGGTGACTGTCTCCAGC |
| hIL27 | 1428 | CAGGTCCAGCTCCAGGAGAGTGGTGGGGGGTCAGTGCAGGCGGGAGGCTCACTG |
| Ra_VH | | CGTCTGTCCTGTCGGGCCTCCGGCTCCACTTACTCTAACTATTGCTTGGGCTGG |
| H16- | | TTCAGGCAGATTACAGGTAAGGAGCGCGAAGGGGTGGCTGTTATCAACTGGGTA |
| DR593 | | GGGGGTATGCTGTATTTCGCCGATTCAGTTAAGGGCCGTTTCACGGTGAGTCAG |
| | | GATCAGGCCAAAAACACTGTTTATCTTCAAATGAACTCCCTGAAGCCCGAGGAC |
| | | ACCGCGATGTACTATTGCGCAGCCGAGAGCGCGTCTAGCTTCAGGTGTGGGGGA |
| | | TGGCTGACACGGCCTGACCGCGTCCCTTATTGGGGCCAGGGAACCCAAGTGACC |
| | | GTGTCTAGCGGAGGCTCCGGCGGATCTGGAGGTTCCGGTCAGGTGCAGTTGCAA |
| | | GAGTCCGGCGGAGGGTCTGTGCAGGGGCGGGGCAGCCTGCGCCTGTCCTGCGCG |
| | | GCCTCTGGATATCCGTACAGCAACGGCTACATGGGCTGGTTCAGACAAGCACCG |
| | | GGGAAAGAGAGAGAGGGCGTGGCCACCATCTACACCGGGGACGGGCGTACTTAC |
| | | TATGCCGACAGTGTAAAGGGGCGCTTCACTATGTCACGCGACAATGCGAAAAAC |
| | | ACAGTGGACCTCCAGATGTCAAGTTTGAAGCCGGAGGACACTGCAATGTATTAC |
| | | TGTGGTGCCAGAGCCGCACCTCTGTACTCCAGCGGCTCTCCTCTGACTCGTGCC |
| | | CGCTACAACGTGTGGGGTCAGGGCACTCAGGTCACAGTCAGCAGT |
| hIL27 | 1429 | CAAGTGCAGCTCCAGGAATCTGGAGGCGGAAGCGTCCAGGCGGGGGGTTCTCTG |
| Ra_VH | | CGTCTGTCTTGTCGGGCCAGCGGCTCTACTTACTCTAATTACTGTCTGGGTTGG |
| H16- | | TTCCGCCAGATCACTGGTAAGGAGAGAGAGGGGGTGGCCGTTATCAACTGGGTC |
| DR594 | | GGCGGGATGTGTACTTTGCTGATTCAGTGAAACGGCCGGTTCACAGTAAGCCAG |
| | | GATCAGGCCAAGAACACAGTTTACCTCCAGATGAACTCTTTGAAGCCTGAGGAC |
| | | ACCGCCATGTACTATTGTGCGGCAGAATCCGCCTCTAGCTTCAGCTGTGGCGGT |
| | | TGGCTGACCCGTCCTGACAGAGTGCCATACTGGGGCCAGGGCACCCAGGTGACA |
| | | GTGTCCTCTGGCGGAGGCTCCCAGGTGCAGCTCCAGGAATCAGGGGGAGGCAGT |
| | | GTGCAGGCGGGTGGCTCCCTGAGATTGTCCCGTGTCGCCAGCGCGTCCACCTAT |
| | | TGCACCTACGATATGCACTGGTACAGACAGGCACCCGGAAAGGGCCGTGAGTTT |
| | | GTGAGTGCTATTGATTCCGACGGTACAACTCGCTACGCGGATTCAGTGAAAGGG |
| | | AGATTCACGATTTCTCAAGGCACGGCCAAGAATACTGTGTACCTCCAGATGAAC |
| | | AGCCTCCAGCCAGAAGACACGGCTATGTACTATTGCAAGACCGTGTGTGTGGTC |
| | | GGCAGCAGGTGGTCCGACTATTGGGGCCAGGGTACACAGGTGACCGTGAGCAGG |
| hIL27 | 1430 | CAAGTGCAACTCCAGGAGAGGGGCGGTGGCTCCGTGCAGGGGGGCGGAAGCCTC |
| Ra_VH | | CGCCTTAGTTGCAGAGCGAGCGGCAGCACCTACTCCAACTACTGCTTGGGCTGG |
| H16- | | TTTAGACAGATTACGGGCAAGGAACGTGAGGGTGTCGCCGTAATCAACTGGGTT |
| DR594 | | GGCGGTATGCTCTACTTTGCTGACTCCGTTAAGGGACGCTTTACTGTTTCTCAG |
| | | GATCAGGCGAAGAACACCGTCTACCTTCAGATGAACTCCCTCAAACCCGAGGAC |
| | | ACCGCTATGTACTATTGCGCAGCCGAATCTGCAAGCTGTTTCTCATGGGGTGGG |
| | | TGGCTGACCCGCCCGGACCGTGTGCCTTACTGGGGCCAGGGGACACAGGTGACC |
| | | GTTAGCTCCGGGGGCTCCGGGGGTTCAGGTGGATCTGGGCAGGTTCAGCTGCAA |
| | | GAGTCAGGTGGGGGCTCTGTCCAAGCCGGGGGATCTCTGAGACTGTCATGCGTG |
| | | GCCTCCGCATCAACCTACTGCACCTATGATATGCACTGGTATCGCCAAGGTCCG |
| | | GGTAAAGGCAGGGAGTTTGTGAGCGCCATCGACAGCGATGGAACGACCCGCTAC |
| | | GCCGATAGCGTGAAGGGGAGATTTACTATTTCCCAAGGAACAGCCAAAAACACA |
| | | GTCTACCTTCAGATGAACTCCCTGCAACCCGAAGACACAGCCATGTATTACTGC |
| | | AAGACAGTTTGCGTGGTTGGTTCCCGTTGGAGCGACTACTGGGGCCAGGGCACC |
| | | CAGGTGACTGTCTCCTCT |
| hIL27 | 1431 | CAGGTCCAGGTGCAAGAATCAGGCGGTGGGTCCGTGCAAGCTGGTGGATCTCTC |
| Ra_VH | | CGTCTGTCTTGCCGCGCTTCTGGAAGCACTTATTCAAATTATTGCCTGGGGTGG |
| H16- | | TTTAGGCAGATTACTGGGAAGGAGCGCGAGGGCGTCGCCGTGATAAATTGGGTC |
| DR595 | | GGTGGGATGCTGTATTTCGCGGACAGCGTGAAGGGCCGGTTCACAGTCTCCCAA |
| | | GATCAGGCTAAGAACACAGTGTACTTGCAGATGAACTCCCTGAAGCCAGAGGAC |
| | | ACTGCCATGTACTATTGCGCGGCTGAAAGCGCCTCTTCCTTCTCCTGTGGGGGG |
| | | TGGCTCACACGCCCAGATCGCGTACCATACTGGGGTCAGGGCACACAGGTGACC |
| | | GTGTCATCTGGGGGGCGGAGCCAGGTGCAGCTCCAGGAGTCAGGAGCGGGGTCC |
| | | GTACAGGCTGGTGGCTCCCTGACCCTGTCTTGCGCTGCATCAGAGTACGCCTAC |
| | | TCCACCTGCAACATGGGTTGGTATCGTCAGGCTCCCGGCAAAGAAAGGGAACTC |
| | | GTGAGTGCCTTCATTAGCGATGGATCAACCTATTACGCGGACTCCGTGAAGGGC |
| | | CGCTTCACCATCACCCGCGACAACGCGAAAAATACGGTCTACTTGCAGATGAAT |
| | | AGTCTGAAACCAGAGGACACAGCCATCTATTACTGCTCTGCCAACTGTTACCGT |
| | | CGCCTGCGGAATTATTGGGGCCAGGGCACCCAGGTGACTGTCTCCTCT |
| hIL37 | 1432 | CAGGTCCAGTTGCAGGAGTCCGGGGGTGGCTCTGTCCAAGCAGGTGGCAGCCTG |
| Ra_VH | | CGGCTGTCTTGCCGCGCGAGCGGGTCCACTTATAGTAACTATTGCCTGGGCTGG |
| H16- | | TTTCGTCAGATCACAGGCAAGGAGCGCGAAGGCGTCGCCGTTATCAACTGGGTG |
| DR595 | | GGCGGGATGGTGTATTTCGCCGACAGCGTGAAGGGACGTTTTACAGTGTCACAG |
| | | GACCAGGCCAAGAACACCGTCTACCTCCAGATGAACTCCCTGAAGCCTGAAGAC |
| | | ACTGCCATGTACTATTGTGCCGCAGAGTCAGCCAGCTCGTTCAGCTGCGGCGGA |
| | | TGGTTGACCCGCCCGGACAGGGTTCCCTACTGGGGTCAGGGCACTCAGGTTACG |
| | | GTGAGCAGTGGTGGCTCTGGTGGCTCTGGAGGCTCCGGCCAGGTGCAGCTTCAG |
| | | GAGAGTGGCGGTGGAAGTGTGCAAGCGGGAGGTAGTCTGACCCTGTCTTGCGCT |
| | | GCCTCTGAATACGCATACAGTACATGCAATATGGGATGGTATCGCCAGGCCCCT |
| | | GGCAAAGAGCGTGAACTCGTGAGCGCCTTCATCTCAGATGGGAGCACCTATTAC |
| | | GCTGACTCTGTCAAGGGCAGGTTCACGATTACCCGCGATAACGCAAAGAACACC |
| | | GTGTATTTGCAGATGAACTCCCTGAAGCCTGAAGACACAGCCATTTATTACTGG |
| | | TCCGCCAACTGTTATCGCAGACTGCGGAACTATTGGGGCCAGGGCACCCAGGTG |
| | | ACCGTGTCTTCC |
| hIL27 | 1433 | CAGGTGCAGTTGCAGGAGTCTGGAGGTGGCTCCGTCCAGGCTGGTGGCTCTCTG |
| Ra_VH | | CGGCTGAGCTGCCGCGCGAGTGGCAGCACCTACTCCAACTACTGGCTGGGCTGG |
| H16- | | TTTCGCCAGATTACAGGAAAGGAGAGAGAGGGAGTGGCCGTCATCAACTGGGTG |
| DR596 | | GGCGGGATGTTGTATTTCGCTGACAGTGTGAAGGGAAGATTTACCGTTAGTCAG |
| | | GACCAGGCTAAGAACACCGTATACCTCCAGATGAACTCTCTGAAGCCAGAGGAT |
| | | ACAGCCATGTACTATTGTGCCGCAGAGTCTGCCTCCTCTTTTTCATGCGGCGGG |
| | | TGGTTGACTCGCCCAGACAGAGTACCATACTGGGGCCAGGGGACCCAAGTGACC |
| | | GTCTCCAGCGGGGGGGGCAGCCAGGTCCAGCTCCAGGAGTCAGGTGGGGGGCTG |
| | | GTCCAGCCTGGCGGTTCTCTGCGCCTGTCCTGCACCGCCAGTGGGCTCACCTTC |
| | | GATGACTCCGTGATGGGTTGGTTCCGGCAGGCTCCGGGTAAGGGACGTGAGGCT |
| | | GTGTCCTGTATTTCTTCCAGCGGAGCCAACGCATTTTATGCGGACAGTGTAAAG |
| | | GGTCGCTTTACCATCAGTCGTGACAACGCCAAGAACACCCTCTATCTTCAGATG |
| | | AACTCCCTGAAGCCAGAGGATACTGCCACGTATTACTGCAAGCGTGGTCATGCG |
| | | TGCGCAGGCTATTACCCCATCCCCTACGACGATTATTGGGGCCAAGGCACGCAG |
| | | GTGACCGTTTCCTCC |
| hIL27 | 1434 | CAGGTGCAGCTTCAGGAGTCCGGTGGGGGCAGCGTGCAAGCTGGAGGCAGTCTG |
| Ra_VH | | AGGCTGAGCTGTCGTGCGTCAGGGTCTACCTACTCCAACTATTGTCTGGGGTGG |
| H16- | | TTTCGCCAGATCACTGGTAAGGAGCGCGAGGGCGTTGCCGTTATCAACTGGGTC |
| DR596 | | GGAGGTATGCTGTACTTCGCTGACTCCGTGAAGGGACGTTTCACTGTGAGCCAG |
| | | GATCAGGCCAAGAACACCGTGTACCTCCAGATGAACAGCCTGAAGCCTGAAGAT |
| | | ACCGCCATGTATTACTGTGCAGCCGAATCCGCCTCTTCCTTTAGCTGTGGGGGC |
| | | TGGCTGACTAGGCCTGACCGCGTGCCTTACTGGGGCCAGGGGACCCAAGTAACC |
| | | GTCAGCTCTGGTGGCAGCGGAGGTAGCGGTGGGAGTGGCCAGGTTCAGCTTCAG |
| | | GAGAGCGGTGGAGGTCTGGTGCAGCCAGGGGGTAGTCTGCGCCTTAGCTGCACC |
| | | GCCTCCGGTCTGACCTTCGATGACAGTGTGATGGGTTGGTTTCGTCAGGCTCCA |
| | | GGCAAGGGGCGCGAAGCAGTGTCTTGTATCAGGTCCAGCGGCGCTAATGCCTTT |
| | | TACGCCGATTCCGTGAAAGGCCGCTTTACGATTAGCCGCGATAACGCCAAGAAC |
| | | ACACTGTATTTGCAGATGAACAGCCTCAAACCCGAAGACACGGCCACCTACTAT |
| | | TGTAAGCGGGGTCATGCCTGCGCTGGGTATTACCCAATTCCCTACGATGACTAT |
| | | TGGGGGCAGGGCACCCAGGTTACTGTCTCCCCC |
| hIL27 | 1435 | CAGGTTCAGTTGCAGGAAAGCGGAGGTGGCTTGGTGCAGCCCGGTGGCTCTCTC |
| Ra_VH | | CGTCTGTCTTGCGCGGCCAGTGGGTTCACATTCTCCCTGAGCGGAATGTCTTGG |
| H17- | | GTGCGTCAGGCCCCAGGCAAAGGTCTGGAATGGGTTAGCGCGATCAGCTCTGGT |
| DR591 | | GGCGCTTCCACCTACTATACGGACAGCGTTAAGGGCAGATTTACTATTTCTCGC |
| | | GACAACGCCAAGAACATGCTTTACCTCCAGTTGAACAGCCTGAAGACCGAAGAT |
| | | ACCGGTATGTATTACTGTGCCAAGGCGGGGTCCGGCTACGGCGATGCCTCCCGT |
| | | ATGACATCCCCTGGCAGCCAGGGCACTCAAGTGACCGTGTCCAGCGGCGGTGGC |
| | | AGTCAAGTCCAGCTCCAGGAGTCTGGGGGTGGATCTGTCCAGGCCGGGGGGTCC |
| | | CTCCGCCTGAGTTGCACTGCGAGCGGTGCTATCGCCAGCGGCTACATCGACTCC |
| | | AGGTGGTGCATGGCCTGGTTCCGCCAGGCTCCGGGAAAAGAGCGTGAGGGTGTT |
| | | GCCGCTATCTGGCCCGGAGGGGGGCTGACCGTCTATGCCGACTGCGTAAAGGGC |
| | | CGGTTCACCATCAGCCGCGACCATGCCAAGAACACCCTGTACCTTCAGATGAAT |
| | | AACTTGAAACCTGAGGACACTGCTATGTATTACTGGGGGGCTGGTAGCCGTCGC |
| | | ATGTGCCCGAGCCTGGAATTTGGGTTCGATTACTGGGGACAGGGCACCCAGGTC |
| | | ACGGTGTCCAGC |
| hIL27 | 1436 | CAGGTGCAGTTGCAAGAAAGTGGCGGGGGTCTGGTACAGCCTGGAGGCTCTCTG |
| Ra_VH | | CGCCTCTCCTGTGCTGCCAGCGGCTTTACTTTTAGCCTGTCAGGTATGTCCTGG |
| H17- | | GTCCGCCAGGCCCCCGGTAAAGGTCTTGAGTGGGTCTCCGCTATTTCCAGCGGA |
| DR591 | | GGTGCCAGTACCTATTACACCGACTCAGTGAAAGGACGTTTCACCATTTCCCGC |
| | | GATAATGCTAAGAATATGTTGTATTTGCAGCTCAACAGCCTGAAGACAGAGGAT |
| | | ACCGGTATGTACTATTGCGCTAAAGGGGGATCTGGTTATGGAGACGCCTCCCGT |
| | | ATGACATCCCCTGGGTCTCAGGGAACGCAGGTGACCGTGTCTAGCCGGGGGTCC |
| | | GGAGGCTCCGGTGGCTCCGGCCAGGTCCAGCTCCAGGAAAGCGGTGGCGGGTCT |
| | | GTGCAGGCAGGCGGTTCCCTGAGGCTGAGCTGCACAGCCTCCGGGGCAATCGCC |
| | | AGCGGTTATATCGACAGCAGATGGTGCATGGCCTGGTTCCGTCAGGCCCCCGGC |
| | | AAAGAGAGGGAGGGAGTGGCGGCCATCTGGCCAGGAGGCGGACTGACCGTGTAT |
| | | GCCGACTCTGTGAAGGGTCGCTTTACCATCAGCAGAGACCACGCCAAGAACACC |
| | | CTGTACTTGCAGATGAATAACCTCAAGCCCGAGGACACCGCAATGTATTACTGC |
| | | GCCGCAGGCAGCCCTCGCATGTGTCCGTCTCTGGAGTTCGGATTCGATTACTGG |
| | | GGCCAGGGGACCCAGGTGACCGTTTCCTCC |
| hIL27 | 1437 | CAAGTGCAGGTCCAGGAGTCAGGTGGAGGCCTGGTTCAGCCTGGGGGAAGTCTG |
| Ra_VH | | CGCCTTTCATGTGCAGCTTCTGGTTTTACCTTCTCTCTGAGCGGCATGTCCTGG |
| H17- | | GTCCGGCAGGCACCGGGCAAGGGCCTGGAGTGGGTGAGTGCCATCTCATCTGGC |
| DR592 | | GGTGCGAGCACCTACTATACCGACTCTGTTAAAGGAAGGTTTACCATCTCCCGC |
| | | GATAACGCTAAAAATATGTTGTACTTGCAGCTCAACTCACTGAAGACCGAAGAC |
| | | ACCGCTATGTATTACTGCGCCAAGGGTGGCTCCGGGTACGGCGATGCCTCCCGC |
| | | ATGACCAGCCCCGGTAGTCAGGGCACTCAGGTCACTGTTAGCTCGGGGGGTGGC |
| | | TCCCAGGTCCAGCTCCAGGAGTCTGGTGGAGGCTCCGTGCAGGCTGGGGGTTCC |
| | | CTGAGATTGTCTTGTACTGCCCCCGGCTTCACAAGTAACTCCTGTGGTATGGAC |
| | | TGGTATCGTCAAGCGCCTGGAAAGGAGCGCGAGTTCGTTAGCTCCATTTCTACC |
| | | GATGGGACCACTGGGTATGCAGACAGCGTGAAAGGTCGCTTTACCATCTCCAAG |
| | | GACAAGGCTAAGGACACCGTGTACCTCCAAATGAACTCCCTGAAGCCCGAGGAC |
| | | ACCGGGATGTATTCCTGCAAGACCAAGGACGGGACCATCGCCACTATGGAGTTG |
| | | TGTGACTTCGGATATTGGGGGCAGGGCACCCAAGTGACTGTGTCTAGC |
| hIL27 | 1438 | CAGGTCCAGCTTCAGGAGTCTGGCGGGGGTCTGGTCCAGCCTGGGGGTTCTCTC |
| Ra_VH | | CGCCTGTCCTGCGCAGCTAGTGGATTCACCTTCTCTCTCTCCGGTATGTCTTGG |
| H17- | | GTGCGCCAGGCCCCTGGCAAGGGGCTGGAGTGGGTGAGTGCAATTAGCTCGGGG |
| DR592 | | GGAGCAAGCACCTACTATACAGACTCAGTCAAGGGAAGATTCACAATTTCCCGC |
| | | GATAACGCTAAAAACATGCTGTATCTCCAGCTCAACAGCCTGAAGACGGAAGAC |
| | | ACGGCTATGTACTATTGTGCCAAAGGGGGCAGGGGCTATGGCGACGCCAGCCGC |
| | | ATGACCTCTCCGGGCAGCCAGGGTACGCAGGTGACCGTGTCCAGCGGAGGGTCC |
| | | GGGGGCTCTGGAGGTTCAGGGCAGGTACAGCTCCAAGAATCCGGGGGGGGTTCT |
| | | GTGCAGGCTGGAGGCTCCCTGCGCCTGAGCTGCACAGCTCCGGGCTTTACCTCC |
| | | AACAGTTGCGGAATGGATTGGTACAGGCAAGCGCCCGGCAAGGAGAGGGAGTTC |
| | | GTGTCCAGTATTTCAACCGACGGCACAACCGGCTACGCGGATTCCGTGAAAGGA |
| | | CGCTTCACCATTTCCAAAGACAAGGCCAAGGACACCGTGTATTTGCAGATGAAC |
| | | TCACTGAAGCCCGAGGATACCGGAATGTATTCTTGTAAAACGAAGGATGGGACC |
| | | ATCGCTACAATGGAGCTGTGTGACTTTGGTTACTGGGGCCAGGGCACTCAGGTC |
| | | ACTGTCTCTTCT |
| hIL27 | 1439 | CAGGTGCAGCTCCAGGAAAGCGGTGGCGGTTTGGTGCAGCCTGGGGGCTCCCTG |
| Ra_VH | | CGCCTGTCTTGTGCGGCTAGTGGCTTCACCTTCTCTCTGTCCGGCATGAGCTGG |
| H17- | | GTGGGGCAAGCGCCGGGCAAGGGCCTCGAATGGGTGAGGGCAATCAGCTCTGGA |
| DR593 | | GGCGCATCAACCTACTATACCGATTCAGTCAAGGGAAGGTTTACCATCTCCAGA |
| | | GATAACGCCAAGAATATGCTGTACCTCCAGCTCAATTCACTGAAGACCGAGGAT |
| | | ACCGCCATGTATTACTGCGCTAAGGGTGGCAGGGGATACGGAGATGCGTCCCGG |
| | | ATGACAAGCCCCGGCTCACAGGGCACACAGGTGACTGTGTCCTCAGGAGGTGGC |
| | | TCCCAAGTGCAGCTTCAGGAGAGCGGTGGAGGCTCCGTCCAAGCTGGAGGGTCC |
| | | CTCCGTCTCTCTTGTGCAGCTTCCGGCTATCCTTACTCCAATGGGTACATGGGA |
| | | TGGTTCCGCCAAGCTCCTGGCAAGGAGAGGGAGGGGGTGGCTACCATCTACACC |
| | | GGCGATGGCCGCACCTATTACGCCGACAGCGTGAAAGGACGTTTCACCATTTCC |
| | | CGTGACAATGCAAAGAACACTGTGGACCTCCAGATGTCAAGCCTCAAGCCAGAA |
| | | GACACCGCGATGTACTATTGTGCTGCCAGAGCAGCGCCCCTGTATTCTTCAGGC |
| | | TCTCCCCTGACACGCGCACGTTACAACGTCTGGGGCCAGGGTACTCAGGTCACC |
| | | GTGTCTAGC |
| hIL27 | 1440 | CAGGTGCAGCTCCAGGAATCCGGGCGGAGGCCTGGTCCAGCCCGGGGGTCTCTG |
| Ra_VH | | CGCCTGTCTTGTGCTGCCAGCGGCTTCACCTTCAGTCTGAGTGGAATGAGCTGG |
| H17- | | GTGCGTCAGGCTCCAGGAAAGGGCCTGGAGTGGGTGTCCGCCATTAGCTCTGGA |
| DR593 | | GGCGCGTCCACATATTACACCGATAGCGTAAAAGGCCGTTTCACTATCTCACGC |
| | | GACAACGCTAAGAATATGCTGTATCTCCAGCTGAACCCCCTGAAAACCGAGGAT |
| | | ACGGCCATGTATTACTGTGCGAAAGGGGGTTCAGGGTACGGCGACGCATCCAGG |
| | | ATGACCAGCCCAGGTTCCCAAGGGACCCAGGTGACAGTGTCTTCCGGGGGAAGC |
| | | GGAGGCTCCGGTGGCAGCGGCCAGGTGCAGTTGCAGGAATCTGGCGGTGGGAGC |
| | | GTGCAGGCTGGCGGGAGCCTGCGCCTGAGCTGTGCTGCGAGCGGCTACCCCTAT |
| | | TCTAACGGGTACATGGGTTGGTTCCGGCAGGCTCCCGGTAAAGAGCGCGAGGGA |
| | | GTGGCCACGATTTACACGGGTGATGGGCGCACTTATTACGCAGACTCCGTTAAG |
| | | GGCCGGTTTACCATTAGTCGTGATAACGCGAAGAACACCGTGGACCTTCAGATG |
| | | TCCAGCTTGAAGCCAGAGGACACCGCTATGTATTACTGTGCTGCACGCGGTGCG |
| | | CCTCTCTACAGTAGCGGCTCCCCTCTGACCCGCGCTCGCTATAACGTGTGGGGC |
| | | CAGGGCACTCAGGTGACAGTAAGCTCC |
| hIL27 | 1441 | CAAGTTCAGCTCCAGGAGTCTGGAGGCGGTCTCGTGCAGCCCGGTGGCTCCCTC |
| Ra_VH | | CGCCTGTCTTGCGCCGCGAGCGGCTTCACCTTGTCTTTGAGCGGAATGAGCTGG |
| H17- | | GTCAGGCAGGCTCCCGGCAAAGGCCTGGAGTGGGTGTCCGCCATTAGTAGCGGC |
| DR594 | | GGAGCCTCTACCTATTACACCGATTCTGTGAAGGGTCGTTTCACCATTTCTCGC |
| | | GATAACGCAAAGAACATGCTGTACCTCCAGCTGAACTCTCTGAAGACCGAGGAT |
| | | ACTGCAATGTATTACTGCGCCAAAGGCGGTAGTGGTTACGGTGACGCCAGCAGG |
| | | ATGACCTCCCCAGGATCACAGGGAACCCAGGTCACCGTCAGCCCTGGTGGAGGG |
| | | AGCCAGGTGCAGTTGCAGGAAAGCGGCGGAGGCAGTGTGCAGGCCGGAGGGTCT |
| | | CTGAGGCTCTCTTGCGTCGCCAGCGCCTCCACTTACTGCACTTATGACATGCAC |
| | | TGGTATCGGCAAGCTCCTGGGAAGGGCCGTGAATTTGTGAGCGCTATCGACAGC |
| | | GACGGCACCACTAGGTATGCTGATTCTGTGAAAGGGAGGTTTACCATTTCCCAG |
| | | GGCACCGCCAAGAACACGGTGTATCTCCAGATGAACTCTCTCCAGCCGGAAGAC |
| | | ACTGCCATGTATTACTGTAAGACCGTGTGCGTGGTGGGATCTCGGTGGTCTGAT |
| | | TATTGGGGCCAGGGAACCCAGGTGACGGTTTCTTCC |
| hIL27 | 1442 | CAGGTCCAGTTGCAGGAGTCCGGCGGAGGGCTGGTACAGCCCGGGGGAAGCCTG |
| Ra_VH | | CGCCTGAGTTGCGCCGCGTCTGGATTTACTTTCTCTCTCTCTGGTATGAGCTGG |
| H17- | | GTGCGCCAGGCTCCCGGCAAGGGACTTGAGTGGGTAAGCGCAATCTCCTCTGGC |
| DR594 | | GGGGCGAGCACCTATTACACCGACAGCGTTAAGGGCAGGTTCACAATTAGCCGT |
| | | GACAACGCTAAGAATATGCTTTATCTTCAGCTGAACAGCCTGAAGACTGAAGAC |
| | | ACGGCCATGTATTACTGTGCCAAGGGCGGTTCCGGCTACGGAGACGCTAGTCGC |
| | | ATGACCTCCCCAGGCTCCCAGGGCACCCAGGTCACCGTGTCTAGGGGAGGCTCT |
| | | GGCGGTAGTGGTGGCTCCGGTCAGGTGCAACTTCAGGAGAGTGGGGGTGGCTCC |
| | | GTTCAAGCTGGGGGTTCCCTGCGTCTGTCTTGTGTCGCTAGTGCGTCCACTTAT |
| | | TGCACCTACGACATGCACTGGTATCGTCAAGCGCCGGGCAAGGGAAGGGAGTTC |
| | | GTCAGCGCCATTGATTCCGACGGCACCACTCGCTACGCCGACTCTGTCAAGGGA |
| | | CGCTTCACCATCTCCCAGGGCACTGCCAAAAATACCGTGTATTTGCAGATGAAC |
| | | TCACTTCAGCCAGAGGACACTGCCATGTATTACTGCAAGACCGTCTGCGTCGTG |
| | | GGCAGCAGGTGGTCTGATTACTGGGGCCAAGGCACCCAGGTGACCGTCAGCAGC |
| hIL27 | 1443 | CAGGTGCAACTTCAGGAATCAGGGGGGGGACTGGTGCAGCCAGGTGGCTCTCTG |
| Ra_VH | | CGCCTCAGCTGTGCGGCCTCTGGCTTTACCTTCTCTTTGAGCGGTATGAGTTGG |
| H17- | | GTGCGCCAGGCTCCGGGAAAAGGTCTGGAGTGGGTCTCTGCCATCTCTTCCGGC |
| DR595 | | GGTGCCTCCACGTACTATACCGACTCCGTGAAGGGCCGGTTTACCATTTCCAGG |
| | | GACAACGCCAAGAACATGCTTTATCTCCAGCTGAACTCTCTGAAGACAGAAGAT |
| | | ACCGCCATGTACTATTGCGCCAAAGGGGCCCCCGGTTACGGCGACGCGAGCCGC |
| | | ATGACTAGCCCTGGCTCCCAGGGAACCCAGGTCACAGTGTCCTCTGGGGGTGGC |
| | | TCTCAGGTCCAGCTCCAGGAGTCTGGTGGGGGGTCCGTGCAGGCCGGTGGCTCC |
| | | CTGACCCTGTCTTGTGCTGCCAGTGAGTATGCGTACAGTACCTGTAATATGGGT |
| | | TGGTACAGGCAAGCGCCCGGCAAGGAACGCGAGCTGGTCTCCGCTTTTATCTCC |
| | | GACGGAAGCACCTATTACGCAGACTCAGTGAAGGGTCGTTTCACCATCACCCGT |
| | | GATAATGCTAAAAACACTGTGTACCTCCAGATGAATAGTCTGAAGCCCGAGGAT |
| | | ACCGCTATTTATTACTGCTCCGCCAACTGTTACCGTCGCCTGAGAAACTACTGG |
| | | GGACAAGGCACCCAGGTAACCGTCAGCTCT |
| hIL27 | 1444 | CAGGTGCAGTTGCAGGAGTCTGGCGGAGGTCTGGTACAGCCTGGTGGCAGCCTC |
| Ra_VH | | CGCTTGAGCTGCGCGGCCAGTGGCTTCACTTTTTCCCTGTCAGGCATGAGCTGG |
| H17- | | GTGCGTCAGGCTCCTGGCAAAGGACTGGAGTGGGTGTCCGCTATGTCCTCTGGA |
| DR595 | | GGCGCATCCACTTATTACACCGATTCTGTCAAAGGTCGTTTCACCATTTCCCGC |
| | | GATAATGCTAAGAACATGCTGTATTTGCAGTTGAACTCCCTGAAGACAGAGGAC |
| | | ACCGGTATGTATTACTGCGCTAAGGGTGGCAGCGGGTATGGGGATGCCAGCCGC |
| | | ATGACAAGCCCCGGCTCCCAGGGTACACAGGTCACCGTCTCCTCAGGAGGCAGC |
| | | GGAGGTAGCGGGGGCTCCGGCCAAGTCCAGCTCCAGGAGTCCGGGGGTGGATCT |
| | | GTCCAGGCCGGGGGCAGTCTCACTCCGTCCTGTGCCGCTTCAGAGTACGCCTAT |
| | | AGTACCTGTAACATGGGCTGGTATAGGCAGGCTCCCGGCAAGGAGAGAGAACTG |
| | | GTCAGCGCGTTTATCTCCGACGGCAGTACCTACTATGCTGACTCTGTGAAGGGA |
| | | CGTTTTACCATCACCCGCGACAACGCCAAGAATACCGTCTACCTCCAGATGAAC |
| | | TCTCTCAAACCGGAGGACACCGCCATCTATTACTGCTCTGCAAACTGTTATAGA |
| | | AGGCTGCGTAACTACTGGGGACAAGGCACGCAAGTGACAGTGTCGTCA |
| hIL27 | 1445 | CAGGTGCAGTTGCAGGAGAGCGGTGGCGGTCTCGTGCAGCCAGGTGGCTCTCTG |
| Ra_VH | | CGGCTGAGTTGCGCTGCCTCTGGCTTCACATTGTCTCTGTCCGGCATGTCTTGG |
| H17- | | GTGCGCCAGGCTCCGGGTAAAGGGCTGGAGTGGGTGAGCGCAATCTCCTCTGGA |
| DR596 | | GGTGCATCCACCTATTACACCGACTCTGTGAAGGGTCGGTTTACGATCTCCCGC |
| | | GACAACGCAAAGAATATGCTTTACCTGCAACTGAACTCCCTGAAAACAGAGGAC |
| | | ACGGCCATGTACTATTGTGCCAAGGGCGGGTCTGGCTACGGCGACGCTTCCAGG |
| | | ATGACCAGTCCGGGCTCTCAGGGCACCCAGGTGACAGTCTCTAGGGGTGGGGGC |
| | | TCCCAGGTCCAGCTTCAGGAAAGCGGAGGTGGCCTTGTTCAACCGGGGGGTTCA |
| | | CTTCGCTTGAGCTGCACAGCTTCAGGTCTGACCTTCGACGATTCTGTCATGGGC |
| | | TGGTTTAGGCAGGCCCCTGGGAAGGGCAGAGAAGCGGTGTCTTGTATCAGTTCC |
| | | TCAGGGGCCAACGCTTTCTATGCAGACTCCGTGAAGGGACGTTTTACAATTTCA |
| | | CGCGATAATGCCAAGAACACATTGTACCTCCAGATGAACTCCCTGAAACCCGAG |
| | | GACACTGCTACATACTATTGTAAACGCGGCCACGCTTGTGCAGGGTACTATCCC |
| | | ATCCCTTACGATGACTACTGGGGTCAGGGAACCCAGGTTACCGTTAGCTCC |
| hIL27 | 1446 | CAGGTTCAGGTCCAGGAGAGCGGAGGTGGCCTGGTTCAGCCTGGTGGCAGCCTG |
| Ra_VH | | CGCCTGTCTTGCGCTGCCAGCGGTTTTACCTTTAGTTTGTCCGGTATGTCATGG |
| H17- | | GTTCGTCAAGCTCCTGGCAAGGGTCTCGAATGGGTGTCCGCGATTTCCTCAGGA |
| DR596 | | GGTGCCTCTACTTATTACACCGATAGTGTGAAGGGTCGGTTTACTATCTCTCGC |
| | | GACAACGCGAAGAACATGCTGTACCTCCAACTGAACAGCCTGAAGACTGAAGAT |
| | | ACCGCGATGTACTATTGCGCGAAGGGAGGCTCTGGGTATGGAGATGCCTCCCGT |
| | | ATGACATCTCCTGGCTCTCAAGGCACCCAGGTGACCGTCTCCAGGGGGGGTTCC |
| | | GGGGGCTCAGGGGGATCTGGCCAGGTTCAGTTGCAGGAGTCCGGCGGTGGCCTG |
| | | GTCCAACCGGGGGGAGCCTGAGGCTGTCCTGCACTGCGAGCCGGGTTGACATTC |
| | | GACGATAGCGTGATGGGTTGGTTTCGCCAGGCTCCCGGAAAGGGTCGGGAGGCC |
| | | GTCAGCTGTATCTCCAGCAGTGGGGCTAATGCCTTCTACGCCGATTCCGTTAAA |
| | | GGGAGATTTACCATCTCACGGGATAACGCTAAGAACACGCTGTACTTGCAGATG |
| | | AACAGTCTCAAGCCAGAGGACACAGCGACTTACTATTGTAAACGCGGACATGCC |
| | | TGTGCCGGGTACTATCCTATTCCCTACGATGACTATTGGGGTCAGGGCACCCAG |
| | | GTGACCGTATCTTCT |
| hIL27 | 1447 | CAGGTTCAGTTGCAAGAGTCCGGCGGTGGAAGCGTCCAGGCCGGGGGTAGTCTT |
| Ra_VH | | CGGCTGTCATGCGTGGCGAGTGGCTATGTGTCTTGCGACTATTTTCTGCCGAGC |
| H18- | | TGGTATCGCCAGGCTCCAGGTAAGGAACGCGAGTTCGTGAGCATCATTGATGGC |
| DR591 | | ACAGGTTCAACATCCTACGCTGCGTCCGTGAAGGGACGGTTCACCGCGAGCCAG |
| | | GACAAGGGTAAGAACATCGCTTACCTCCAGATGAACTCCCTGAAGCCGGAGGAC |
| | | ACCGCCATGTACTATTGTAAGGCTTCCTGTGTGCGCGGCAGGGGAATCAGTGAA |
| | | TACTGGGGCCAAGGAACACAGGTGACTGTCAGTTCCGGTGGCGGAAGCCAGGTG |
| | | CAGTTGCAAGAGTCGGGGGGCGGTTCCGTGCAGGCTGGTGGCTCCCTGAGACTC |
| | | TCTTGTACCGCTTCAGGTGCTATTGCCAGTGGATACATTGATTCCCGCTGGTGC |
| | | ATGGCCTGGTTCCGCCAGGCCCCCGGCAAGGAGAGGGAAGGAGTCGCCGCTATC |
| | | TGGCCTGGGGGTGGCCTGACCGTGTATGCTGATAGCGTGAAGGGCAGATTCACC |
| | | ATCAGCCGCGATCATGCTAAGAACACACTCTACCTTCAGATGAATAACCTGAAG |
| | | CCTGAGGACACCGCTATGTATTACTGCGCAGCCGGTTCCCCTCGCATGTGCCCC |
| | | TCTTTGGAGTTTGGCTTCGACTACTGGGGCCAGGGCACTCAGGTTACCGTAAGC |
| | | TCC |
| hIL27 | 1448 | CAAGTCCAGCTCCAGGAATCCGGGGGGGCTCCGTCCAGGCGGCGGGGTTCCCTC |
| Ra_VH | | AGGCTCTCCTGCGTGGCCAGCGGCTACGTAAGCTGCGATTATTTCCTGCCATCC |
| H18- | | TGGTATCGCCAAGCCCCTGGTAAGGAGAGAGAATTTGTCTCAATCATTGATGGT |
| DR591 | | ACAGGTAGCACCTCCTACGCAGCCTCAGTGAAGGGACGTTTTACCGCGAGCCAA |
| | | GACAAAGGCAAGAACATCGCGTATTTGCAGATGAACTCTCTGAAGCCGGAGGAT |
| | | ACCGCGATGTACTATTGCAAAGCCTCCTGTGTACGCGGCAGGGGAATCTCAGAG |
| | | TATTGGGGCCAGGGCACTCAAGTGACAGTCAGCTCTGGGGGGAGCGGGGGGTCT |
| | | GGAGGTAGCGGTCAGGTGCAACTCCAGGAATCTGGCGGAGGCTCTGTGCAGGCC |
| | | GGTGGCAGCCTGCGTCTCAGCTGCACAGCCAGCGGGGCCATTGCCAGCGGCTAC |
| | | ATCGACTCCCGCTGGTGCATGGCTTGGTTCCGGCAAGCGCCGGGAAAGGAAAGA |
| | | GAGGGAGTCGCCGCGATCTGGCCGGGGGGTGGCCTGACTGTGTACGCTGACAGC |
| | | GTGAAGGGTCGCTTCACCATCAGTCGGGACCATGCGAAAAACACGCTGTACTTG |
| | | CAGATGAACAATCTGAAGCCTGAGGACACTGCCATGTATTACTGCGCTGCCGGT |
| | | AGTCCACGCATGTGCCCGTCTCTGGAGTTCGGCTTTGACTACTGGGGCCAGGGG |
| | | ACTCAGGTCACGGTTAGCTCT |
| hIL27 | 1449 | CAGGTGCAACTGCAAGAAAGTGGTGGAGGTTCCGTGCAAGCTGGCGGATGTTTG |
| Ra_VH | | CGCCTCAGCTGCGTGGCCTCTGGCTATGTGAGTTGTGATTACTTCCTGCCTTCT |
| H18- | | TGGTATCGTCAGGCCCCTGGTAAGGAACGGGAGTTCGTGTCCATTATCGACGGC |
| DR592 | | ACCGGATCTACGAGCTACGCTGCATCCGTCAAGGGTCGTTTTACCGCTTCCCAA |
| | | GACAAAGGCAAAAATATTGCCTACTTGCAAATGAACTCTCTGAAACCCGAAGAT |
| | | ACAGCAATGTATTACTGTAAAGCCAGTTGTGTGCGCGGACGCGGCATTTCCGAG |
| | | TATTGGGGACAGGGCACTCAGGTGACTGTCTCATCTGGGGGGGGCTCCCAGGTG |
| | | CAACTTCAGGAATCTGGCGGGGGTTCTGTTCAAGCAGGCGGTTCTCTGCGTCTG |
| | | AGTTGCACCGCTCCCGGATTTACCTCCAACAGTTGCGGAATGGATTGGTATCGC |
| | | CAGGCTCCTGGCAAGGAGCGTGAGTTCGTAAGTAGCATTTCAACTGATGGTACT |
| | | ACCGGATACGCTGACTCAGTTAAGGGTCGGTTCACTATCAGCAAGGATAAAGCT |
| | | AAGGACACAGTGTACCTTCAGATGAACTCCCTGAAGCCTGAGGACACCGGAATG |
| | | TACTCCTGTAAAACCAAAGACGGGACCATCGCCACGATGGAACTGTGTGATTTC |
| | | GGATATTGGGGTCAGGGCACCCAGGTGACCGTGAGTAGC |
| hIL27 | 1450 | CAGGTGCAGGTTCAAGAGAGCGGCGGGGGCTCCGTCCAGGCCGGTGGGTCCTTG |
| Ra_VH | | CGCTTGTCTTGTGTGGCCAGCGGATACGTGTCTTGCGATTATTTCCTGCCCTCT |
| H18- | | TGGTACAGACAGGCCCCAGGCAAGGAGCGCGAGTTCGTATCCATCATTGACGGC |
| DR592 | | ACTGGTTCTACAAGTTACGCTGCATCTGTGAAGGGCCGGTTTACCGCCTCTCAG |
| | | GATAAGGGTAAAAACATCGCTTATTTGCAGATGAACTCACTCAAGCCTGAGGAC |
| | | ACCGCCATGTACTATTGTAAGGCCTCTTGTGTCAGAGGGAGAGGCATTAGTGAA |
| | | TATTGGGGTCAGGGGACCCAGGTGACTGTGTCCAGCGGGGGGAGCGGGGGCAGC |
| | | GGTGGCAGCGGCCAGGTTCAACTTCAGGAGAGCGGGGGTGGAAGCGTCCAAGCA |
| | | GGCGGGTCCCTGCGCTTGAGTTGTACCGCTCCGGGATTCACTAGCAACAGCTGC |
| | | GGGATGGACTGGTATCGGCAGGCCCCAGGGAAGGAGCGTGAGTTCGTCAGGTCC |
| | | ATCTCTACAGACGGCACAACCGGCTACGCCGACAGCGTTAAGGGCAGGCTCACA |
| | | ATCAGCAAGGACAAGGCCAAGGATACCGTGTATTTGCAGATGAACAGCCTGAAG |
| | | CCAGAGGATACAGGAATGTATAGCTGCAAGACTAAGGACGGCACTATTGCAACT |
| | | ATGGAACTGTGTGATTTTGGCTACTGGGGTCAAGGCACGCAGGTGACCGTGAGT |
| | | TCC |
| hIL27 | 1451 | CAGGTGCAGGTCCAGGAGTCCGGTGGGGGCTCCGTACAGGGGGGGGGTTCCCTG |
| Ra_VH | | CGCCTTTCTTGTGTGGCCAGCGGTTACGTGAGCTGCGACTACTTCCTCCGTTCC |
| H18- | | TGGTATCGTCAAGCCCCTGGTAAAGAGAGGGAGTTCGTGTCAATCATTGATGGC |
| DR593 | | ACCGGCTCTACCTCTTATGCTGCCTCTGTGAAAGGACGGTTTACTGCTTCTCAG |
| | | GATAAGGGCAAGAATATTGCTTACTTGCAGATGAACAGTCTGAAACCTGAGGAT |
| | | ACTGCCATGTACTATTGCAAGGCCTCCTGTGTGCGTGGCCGGGGCATTAGCGAA |
| | | TACTGGGGCCAGGGAACCCAGGTCACCGTCAGGTCTGGGGGTGGGTCTCAGGTG |
| | | CAGCTGCAAGAATCCGGCGGTGGCTCCGTTCAGGCTGGCGGTTCCCTGCGGCTG |
| | | TCCTGTGCAGCCTCCGGCTACCCGTACTCCAACGGCTATATGGGCTGGTTTCGC |
| | | CAGGCCCCTGGCAAAGAGCGCGAGGGAGTTGCCACCATGTACACCGGGGATGGC |
| | | CGCACTTATTACGCTGACTCTGTGAAAGGGCGCTTCACTATCAGCCGGGATAAG |
| | | GCCAAGAATACCGTGGACCTCCAAATGAGTTCCCTCAAGCCGGAAGACACTGCA |
| | | ATGTACTATTGCGCGGCTCGTGCAGCTCCACTGTACTCCTCTGGGTCTCCCCTG |
| | | ACCCGCGCACGTTATAATGTCTGGGGCCAGGGCACCCAGGTGACCGTCTCAAGT |
| hIL27 | 1452 | CAGGTGCAGCTGCAAGAATCTGGGGGGGGCTCCGTGCAGGCAGGGGGTAGCCTG |
| Ra_VH | | AGGCTGAGCTGCGTGGCTAGTGGCTACGTCTCCTGCGACTACTTTCTGCCCAGC |
| H18- | | TGGTATCGTCAGGCACCTGGGAAGGAACGCGAGTTCGTATCCATCATTGATGGC |
| DR593 | | ACTGGCTCTACCAGCTACGCAGCCTCCGTGAAAGGACGCTTCACTGCGAGCCAG |
| | | GACAAGGGCAAGAACATCGCCTACTTGCAGATGAACTCCCTGAAGCCGGAAGAC |
| | | ACCGCCATGTATTACTGTAAGGCTTCTTGTGTACGCGGGCGCGGTATCTCTGAA |
| | | TATTGGGGCCAGGGCACTCAGGTGACGGTGTCCAGCGGAGGTAGCGGGGGCTCC |
| | | GGCGGGAGCGGGCAGGTTCAGCTCCAGGAGTCTGGGGGTGGCTCCGTGCAGGCC |
| | | GGAGGCTCCCTGCGCCTGTCCTGCGCCGCTTCAGGTTACCCTTATTCTAACGGT |
| | | TATATGGGTTGGTTCCGTCAAGCGCCCGGCAAAGAAAGGGAGGGTGTGGCTACC |
| | | ATTTACACTGGTGATGGTCGCACCTACTATGCTGATTCCGTGAAGGGCCGTTTC |
| | | ACCATCTCTCGCGATAACGCTAAGAATACCGTGGACTTGCAGATGAGTTCTCTG |
| | | AAACCTGAAGATACCGCCATGTACTATTGTGCCGCACGCGCCGCTCCGCTCTAC |
| | | TCTAGCGGGTCTCCCCTGACGAGGGCTAGGTACAACGTGTGGGGACAGGGCACC |
| | | CAGGTGACCGTGTCCAGC |
| hIL27 | 1453 | CAGGTGCAGTTGCAGGAAAGCGGGGGGGGCAGCGTCCAGGCTGGGGGAAGTCTC |
| Ra_VH | | CGCCTGTCCTGCGTGGCCAGTGGCTACGTCAGCTGTGACTATTTTCTGCCGAGC |
| H18- | | TGGTACAGGCAGGCCCCTGGGAAGGAGCGGGAGTTCGTGTCTATCATTGATGGG |
| DR594 | | ACCGGCTCCACCAGCTACGCGGCCTCCGTGAAAGGACGTTTCACGGCCAGCCAG |
| | | GATAAGGGAAAGAATATCGCTTACCTCCAGATGAACTCTCTGAAACCTGAGGAT |
| | | ACTGCCATGTATTACTGCAAGGCCTCATGTGTGCGCGGACGCGGCATCTCAGAA |
| | | TATTGGGGTCAGGGAACCCAGGTGACTGTCAGTTCTGGAGGTGGCTCCCAGGTA |
| | | CAGCTCCAGGAGTCCGGTGGGGGCTCCGTGCAAGCAGGGGGTTCTTTGCGGCTT |
| | | TCCTGTGTAGCCAGCGCCTTCTACCTACTGCACCTACGACATGCCTGGTATCGC |
| | | CAAGCGCCGGGCAAAGGCCGCGAGTTTGTCTCTGCCATTGATTCCGATGGTACA |
| | | ACCCGTTATGCAGATTCCGTGAAGGGCAGATTCACCATCAGCCAGGGCACGGCG |
| | | AAGAATACGGTCTACTTGCAGATGAACTCTTTGCAGCCCGAGGACACAGCCATG |
| | | TATTACTGTAAGACAGTTTGTGTGGTAGGTTCTCGCTGGTCAGATTACTGGGGT |
| | | CAGGGCACACAGGTGACGGTTTCTAGT |
| hIL27 | 1454 | CAGGTCCAGCTTCAGGAGTCTGGGGGGGGTTCCGTTCAGGCAGGCGGGTCCCTC |
| Ra_VH | | AGGCTCTCTTGTGTCGCCTCCGGCTACGTGTCTTGCGACTATTTCCTGCCCTCT |
| H18- | | TGGTATCGGCAGGCTCCGGGCAAGGAGCGCGAGTTCGTGAGCATCATTGATGGC |
| DR594 | | ACAGGCAGTACTTCCTATGCGGCATCCGTGAAAGGGCGCTTCACTGCCAGTCAA |
| | | GACAAGGGCAAAAACATTGCATACCTCCAGATGAACTCCCTGAAGCCTGAAGAC |
| | | ACAGCTATGTATTACTGTAAGGCGAGTTGTGTACGTGGACGTGGTATCAGTGAA |
| | | TACTGGGGCCAGGGCACCCAGGTGACCGTGTCAAGCGGGGGTTCTGGGGGGTCA |
| | | GGCGGTTCCGGCCAGGTGCAGCTCCAGGAGTCCGGGGGAGGGAGCGTCCAGGCC |
| | | GGCGGGTCTCTGCGCCTGTCATGCGTCGCCAGCGCATCTACTTACTGTACCTAC |
| | | GATATGCACTGGTATCGCCAAGCGCCCGGCAAGGGCCGCGAGTTTGTCTCCGCC |
| | | ATTGATTCAGACGGGACAACCCGGTATGCCGATAGTGTGAAGGGCCGTTTTACC |
| | | ATCAGTCAGGGCACTGCTAAGAACACTGTGTATCTCCAGATGAACAGTCTTCAG |
| | | CCTGAAGATACTGCTATGTATTACTGCAAAACTGTGTGTGTGGTTGGATCTCGT |
| | | TGGAGCGACTATTGGGGTCAGGGTACTCAGGTCACAGTGTCTTCT |
| hIL27 | 1455 | CAAGTTCAGTTGCAGGAGTCTGGAGGTGGGTCTGTGCAGGCTGGGGGCTCCCTG |
| Ra_VH | | CGTTTGTCTTGTGTTGCGAGGGGCTACGTCTCTTGTGACTATTTCCTGCCTAGC |
| H18- | | TGGTATCGGCAGGCCCCCGGCAAAGAACGCGAGTTTGTGAGCATTATCGACGGT |
| DR595 | | ACTGGCTCCACCAGCTATGCGGCCTCCGTGAAGGGACGGTTTACCGCCTCCCAG |
| | | GATAAGGGCAAGAACATCGCGTACCTCCAGATGAACTCACTGAAGCCAGAGGAC |
| | | ACCGCTATGTACTATTGCAAAGCAAGCTGTGTTCGCGGTAGGGGAATTTCAGAG |
| | | TATTGGGGCCAAGGAACCCAGGTCACCGTCAGCAGTGGAGGTGGCAGCCAGGTG |
| | | CAGCTCCAGGAGAGTGGCGGGGGTTCTGTGCAGGCCGGAGGCTCCCTGACCCTG |
| | | TCTTGCGCCGCAAGTGAGTATGCGTACTCCACATGCAACATGGGATGGTATCGC |
| | | CAGGCTCCCGGTAAGGAGCGCGAGCTGGTGAGCGCTTTTATCTCAGACGGCTCT |
| | | ACCTACTATGCCGACTCCGTCAAGGGCCGTTTCACAATCACCCGTGATAACGCC |
| | | AAGAATACCGTCTACTTGCAGATGAACTCTTTGAAGCCTGAGGACACAGCCATT |
| | | TACTATTGCTCTGCGAACTGTTACCGCCGTCTGCGGAACTATTGGGGCCAGGGG |
| | | ACCCAGGTGACTGTGTCCTCC |
| hIL27 | 1456 | CAGGTGCAGCTGCAAGAGTCCGGCGGTGGCAGTGTCCAAGCTGGGGGGTCCCTG |
| Ra_VH | | AGGTTGTCCTGCGTGGCATCCGGCTATGTTAGTTGTGACTACTTTCTGCCCTCC |
| H18- | | TGGTATCGCCAGGCTCCTGGCAAGGAGCGCGAATTTGTGTCTATCATTGATGGC |
| DR595 | | ACCGGCAGCACTTCTTATGCTGCATCTGTGAAAGGACGGTTCACCGCGAGCCAG |
| | | GACAAGGGTAAGAATATCGCCTACCTCCAGATGAACAGCCTGAAACCCGAGGAT |
| | | ACTGCCATGTACTATTGTAAGGCCAGTTGCGTGCGCGGTCGTGGCATCAGCGAG |
| | | TATTGGGGCCAGGGGACACAGGTGACCGTGTCCAGTGGAGGTAGCGGTGGAAGC |
| | | GGCGGTTCCGGCCAGGTCCAGGTCCAGGAGAGGGGGGGAGGCTCAGTGCAGGCC |
| | | GGAGGCAGCCTGACACTGTCCTGCGCCGCAAGCGAATATGCGTACAGCACCTGC |
| | | AACATGGGCTGGTATCGCCAGGCCCCAGGCAAGGAGCGTGAACTTGTGTCAGCC |
| | | TTCATCAGCGACGGTAGCACATATTACGCTGACTCAGTGAAGGGACGCTTCACA |
| | | ATCACCAGGGATAACGCCAAGAACACAGTTTACCTCCAGATGAACTCCCTGAAG |
| | | CCAGAGGATACCGCTATTTATTACTGTTCCGCAAATTGTTACCGTAGACTGCGC |
| | | AATTATTGGGGACAAGGCACACAGGTGACAGTGAGTTCT |
| hIL27 | 1457 | CAGGTCCAGTTGCAGGAGTCTGGGGGAGGCAGCGTCCAGGCCGGGGGCAGTCTC |
| Ra_VH | | CGCCTGTCCTGTGTCGCGAGCGGGTACGTGTCTTGCGATTACTTCCTCCCCTCC |
| H18- | | TGGTATCGCCAAGCGCCAGGCAAGGAACGCGAGTTCGTTTCCATCATTGACGGC |
| DR596 | | ACCGGAAGCACTTCCTACGCTGCCTCCGTGAAGGGGCGTTTCACAGCGTCCCAG |
| | | GATAAGGGAAAGAATATCGCTTATTTGCAGATGAACTCTCTGAAGCCTGAGGAT |
| | | ACCGCCATGTACTATTGTAAAGCCTCCTGTGTGAGGGGTCGCGGGATCTCTGAG |
| | | TACTGGGGCCAAGGAACTCAGGTGACGGTGTCTAGCGGCGGAGGGTCCCAGGTG |
| | | CAGCTGCAAGAATCTGGCGGAGGCTTGGTGCAGCCGGGAGGCTCACTGCGTCTC |
| | | TCCTGCACCGCCAGCGGCCTCACTTTTGACGATAGCGTGATGGGATGGTTCCGT |
| | | CAGGCCCCTGGCAAGGGCCGTGAGGCGGTCAGCTGCATCTCTTCCAGTGGTGCC |
| | | AACGCTTTCTACGCAGACAGCGTGAAGGGTAGGTTCACCATCAGCAGAGATAAC |
| | | GCCAAGAACACGCTGTATCTCCAGATGAACTCCCTGAAGCCTGAGGACACTGCC |
| | | ACCTACTATTGCAAGGGGGGCCATGCGTGTGCTGGCTATTACCCAATTCCCTAC |
| | | GATGACTACTGGGGCCAGGGCACACAAGTCACGGTAAGCAGC |
| hIL27 | 1458 | CAGGTCCAGCTCCAGGAATCAGGTGGCGGAAGCGTGCAGGCTGGGGGCTCTCTG |
| Ra_VH | | CGCCTGTCTTGTGTGGCCAGTGGCTACGTTAGCTGCGATTATTTCCTGCCATCT |
| H18- | | TGGTACAGGCAGGCCCCTGGCAAGGAGCGTGAGTTCGTCTCTATCATTGATGGA |
| DR596 | | ACAGGTTCCACCAGTTACGCCGCTTCTGTTAAGGGCCGGTTCACCGCCTCACAG |
| | | GATAAGGGCAAGAACATCGCTTACCTCCAAATGAACTCCCTGAAGCCCGAGGAC |
| | | ACTGCCATGTATTACTGTAAGGCTTCTTGTGTGCGCGGTCGCGGCATTTCCGAA |
| | | TACTGGGGACAAGGAACCCAGGTAACAGTGTCCTCTGGAGGCAGTGGAGGCTCA |
| | | GGGGGCTCTGGTCAGGTCCAACTCCAGGAGAGCGGCGGTGGACTGGTGCAGCCC |
| | | GGTGGCTCTGTCAGGCTGTCCTGTACGGCGAGCGGCCTCACCTTTGATGACTCT |
| | | GTTATGGGCTGGTTCCGCCAAGCCCCTGGGAAGGGGGGGGAGGCTGTGAGCTGT |
| | | ATTTCCTCTAGCGGGGCGAACGCTTTCTACGCCGATTCAGTGAAAGGCCGTTTC |
| | | ACCATCAGTCGCGACAACGCCAAAAACACGCTGTACTTGCAGATGAACTCTTTG |
| | | AAACCAGAGGACACCGCAACTTACTATTGTAAACGCGGCCACGCATGTGCTGGG |
| | | TATTACCCTATCCCCTACGATGACTACTGGGGCCAGGGCACCCAGGTTACAGTG |
| | | TCATCT |
| hIL27 | 1459 | CAAGTCCAGTTGCAGGAATCCGGCGGTGGATCAGTGCAGGCAGGGGGTAGCTTG |
| Ra_VH | | AGGCTGTCTTGTAGAGCGTCCGGTTCAACTTACTCCAACTACTGCCTGGGCTGG |
| H19- | | TTTCGGCAGATTACAGGCAAGGAGCGCGAGGGAGTGGCCGTGATAAATTGGGTG |
| DR591 | | GGTGGAATGCTGTATTTTGCCGACTCCGTGAAGGGCCGGTTCACCGTGTCTCAG |
| | | GACCAGGCCAAGAACACCGTCTACCTTCAGATGAACTCCCTGAAGCCTGAGGAT |
| | | ACCGCCATGTATTACTGCGCCGCTGAGAGTGTCAGCTCTTTTAGTTGCGGTGGG |
| | | TGGCTGACCGGCCCCGACCGTGTGCCGTACTGGGGCCAGGGAACACAGGTGACA |
| | | GTGAGTTCCGGTGGGGGCTCCCAAGTGCAACTTCAGGAGTCTGGCGGTGGCAGC |
| | | GTGCAGGCAGGGGGCTCTCTGCGTCTGTCATGCACCGCCAGCGGAGCCATCGCC |
| | | TCCGGTTACATCGACTCCCGCTGGTGTATGGCTTGGTTTCGCCAGGCTCCCGGC |
| | | AAGGAGCGTGAAGGCGTGGCCGCGATCTGGCCTGGCGGGGGCTGACTGTTTTAT |
| | | GCTGACTCAGTGAAGGGTCGCTTTACCATCTCCCGCGACCACGCAAAGAACACC |
| | | CTTTATTTGCAGATGAATAACCTGAAGCCGGAAGACACTGCCATGTACTATTGC |
| | | GCAGCTGGCTCTCCCCGCATGTGTCCGTCTCTGGAGTTCGGTTTCGATTACTGG |
| | | GGTCAGGGCACTCAGGTTACCGTGTCCAGC |
| hIL27 | 1460 | CAGGTGCAGTTGCAGGAAAGTGGAGGGGGGTCAGTTCAGGCTGGGGGTTCCCTG |
| Ra_VH | | AGGCTGTCCTGTCGTGCAAGTGGCTCAACGTATTCAAACTATTGCCTTGGTTGG |
| H19- | | TTTAGGCAGATCACCGGCAAGGAGCGCGAGGGCGTTGCTGTCATCAACTGGGTC |
| DR591- | | GGCGGGATGCTGTACTTTGCCGACAGCGTCAAGGGCAGATTTACCGTCTCCCAG |
| | | GATCAGGCCAAGAACACAGTGTATCTCCAGATGAACTCTTTGAAGCCCGAAGAC |
| | | ACAGCAATGTACTATTGTGCGGCTGAATCTGTTAGCTCCTTTTCCTGCGGCGGT |
| | | TGGTTGACGAGACCGGATCGGGTCCCGTACTGGGGTCAGGGGACCCAGGTCACG |
| | | GTCAGCTCCGGGGGCAGCGGCGGTAGTGGAGGCTCCGGTCAGGTGCAGCTCCAG |
| | | GAGAGCGGGGGGGGTAGCGTGCAAGCAGGGGGCAGTCTGCGGCTGAGCTGTACT |
| | | GCGAGCGGAGCCATCGCCAGGGGATATATCGACAGCCGGTGGTGTATGGCCTGG |
| | | TTCCGCCAGGCTCCCGGAAAAGAGCGCGAGGGAGTCGCTGCGATTTGGCCAGGG |
| | | GGCGGATTGACAGTATACGCCGACTCTGTGAAAGGCAGATTTACTATCAGCCGC |
| | | GATCACGCCAAGAACACTCTGTACCTCCAGATGAATAACCTCAAACCGGAGGAC |
| | | ACCGCCATGTATTACTGCGCTGCCGGGTCCCCCAGGATGTGTCCTAGTCTGGAG |
| | | TTCGGTTTCGACTACTGGGGCCAAGGAACCCAGGTGACCGTCTCTAGC |
| hIL27 | 1461 | CAGGTGCAGTTGCAGGAGTCAGGAGGTGGGAGTGTTCAGGCCGGAGGCAGCCTG |
| Ra_VH | | CGCTTGAGCTGCCGGGCGTCTGGCAGCACCTACAGCAACTATTGCCTGGGTTGG |
| H19- | | TTCCGTCAGATCACCGGCAAGGAGCGGGAGGGCGTGGCTGTCATCAACTGGGTG |
| DR592 | | GGTGGAATGGTGTATTTTGCGGATAGTGTGAAGGGACGGTTCACAGTCTCTCAG |
| | | GATCAGGCAAAGAATACCGTGTACCTTCAGATGAACTCCCTGAAGCCTGAAGAC |
| | | ACTGCCATGTACTATTGCGCCGCTGAGAGCGTGTCCAGGTTCTCTTGCGGTGGC |
| | | TGGCTCACCAGGCCTGACCGTGTGCCTTACTGGGGCCAGGGTACGCAGGTGACC |
| | | GTATCTAGCGGGGGGGGTTCTCAGGTCCAGTTGCAGGAGTCCGGTGGGGGGTCC |
| | | GTCCAGGGGGGCGGTAGCCTGCGGCTGAGTTGCACCGCTCCTGGATTTACCAGC |
| | | AACAGTTGTGGCATGGACTGGTATCGTCAGGCCCCCGGAAAGGAACGCGAGTTC |
| | | GTGAGCAGTATCTCCACAGACGGCACGACCGGGTACGCTGACTCCGTCAAGGGC |
| | | CGTTTTACCATCTCCAAAGACAAAGCAAAGGATACAGTGTATCTCCAGATGAAC |
| | | TCACTGAAGCCCGAGGACACGGGTATGTATAGCTGCAAGACTAAGGACGGAACC |
| | | ATTGCCACAATGGAGCTTTGTGACTTCGGGTACTGGGGACAGGGAACACAAGTA |
| | | ACGGTCAGCTCC |
| hIL27 | 1462 | CAGGTCCAGTTGCAGGAATCCGGCGGGGGATCAGTGCAGGCTGGCGGGTCTCTG |
| Ra_VH | | CGCCTCTCATGCAGAGCCTCCGGCTCCACCTATTCTAACTACTGCTTGGGCTGG |
| H19- | | TTCAGACAGATCACCGGCAAAGAGCGCGAGGGGGTTGCAGTGATAAATTGGGTG |
| DR592 | | GGTGGAATGCTGTATTTTGCAGACTCCGTCAAGGGAAGGTTCACCGTGAGTCAG |
| | | GATCAAGCCAAGAACACCGTGTATCTGCAAATGAACTCTCTGAAGCCCGAGGAC |
| | | ACCGGTATGTATTACTGTGCAGCTGAAAGCGTGTCCTCTTTCTCTTGCGGAGGC |
| | | TGGCTTACTAGACCTGATCGTGTGCCATATTGGGGTCAGGGCACCCAGGTCACA |
| | | GTTTCTTCCGGTGGCTCAGGCGGATCTGGGGGGTCCGGGCAGGTACAGCTCCAG |
| | | GAGTCTGGTGGCGGTTCCGTTCAGGCTGGGGGGTCCCTGCGTCTGTCCTGCACC |
| | | GCGCCCGGCTTCACAAGCAACTCCTGGGGTATGGACTGGTATCGCCAAGCGCCT |
| | | GGAAAAGAGCGCGAGTTCGTCTCTAGCATTTCCACTGACGGTACTACCGGCTAC |
| | | GCCGACTCCGTGAAGGGGCGCTTCACTATTTCTAAGGATAAGGCGAAGGATACT |
| | | GTGTACCTCCAGATGAACTCACTGAAGCCCGAGGACACTGGGATGTATAGTTGG |
| | | AAGACCAAGGATGGCACAATCGCCACTATGGAATTGTGTGACTTCGGCTACTGG |
| | | GGGCAGGGCACCCAGGTGACAGTGTCCAGC |
| hIL27 | 1463 | CAGGTCCAGTTGCAGGAGTCCGGTGGCGGTTCCGTCCAAGCCGGAGGCTCACTC |
| Ra_VH | | AGACTCTCTTGTCGCGCCTCAGGAAGCACTTACTCCAACTACTGCCTGGGCGGG |
| H19- | | TTTCGCCAGATCACTGGCAAGGAGCGGGAGGGTGTCGCGGTAATAAATTGGGTC |
| DR593 | | GGCGGTATGCTGTATTTCGCGGACTCCGTGAAGGGCCGTTTCACCGTATCACAG |
| | | GACCAGGCCAAGAACACTGTGTATCTCCAGATGAACTCACTGAAGCCCGAAGAT |
| | | ACTGCAATGTATTACTGCGCGGCTGAGAGCGTGTCTTCCTTCTCTTGCGGCGGA |
| | | TGGCTCACACGGCCAGACCGTGTGCCATACTGGGGTCAGGGCACCCAAGTTACA |
| | | GTCTCCAGCGGGGGTGGCTCCCAAGTCCAGCTGCAAGAGAGCGGGGGGGGCTCC |
| | | GTGCAGGCTGGAGGTTCTCTGCGTTTGTCCTGTGCTGCCTCAGGATACCCTTAT |
| | | AGTAACGGTTACATGGGCTGGTTTCGGCAGGCTCCAGGGAAGGAAAGGGAGGGG |
| | | GTGGCTACAATTTACACCGGCGACGGAAGGACCTATTACGCCGACTCTGTGAAA |
| | | GGTCGCTTCACCATTTCCCGTGACAACGCGAAGAATACAGTTGATCTTCAGATG |
| | | TCTTCCCTGAAGCCCGAGGACACAGCGATGTATTACCGCGCTGCCCGTGGTGCC |
| | | CCACTCTACAGCTCTGGCTCTCCCCTGACCCGCGCCCGTTACAACGTGTGGGGC |
| | | CAGGGGACTCAGGTCACAGTCTCATCC |
| hIL27 | 1464 | CAAGTGCAGCTGCAAGAGAGCGGAGGTGGATCTGTGCAGGCTGGTGGGTGTCTG |
| Ra_VH | | CGCCTCTCTTGCCGTGCGTCAGGCTCCACCTACTCTAATTATTGCCTCGGTTGG |
| H19- | | TTCCGGCAGATTACGGGCAAGGAGCGCGAGGGTGTGGCAGTTATCAACTGGGTT |
| DR593 | | GGGGGTATGTTGTACTTTGCTGACTCCGTCAAGGGACGTTTCACTGTGAGTCAA |
| | | GACCAGGCTAAGAACACTGTGTACCCCCAGATGAATAGTCTGAAGCCTGAGGAT |
| | | ACCGCCATGTATTACTGCGCAGCTGAGAGCGTGAGCAGTTTTTCCTGTGGCGGA |
| | | TGGCTTACCAGACCTGATCGCGTGCCGTACTGGGGCCAGGGGACCCAGGTGACT |
| | | GTTAGTTCTGGAGGCAGCGGAGGCTCCGGTGGCAGCGGTCAGGTACAGCTCCAG |
| | | GAATCTGGTGGCGGTAGCGTGCAGGCAGGGGGCTCTCTTCGCCTGAGCTGTGCA |
| | | GCTTCTGGATACCCATATAGCAACGGTTACATGGGCTGGTTTCGCCAAGCCCCC |
| | | GGCAAGGAGAGAGAAGGTGTGGCTACCATTTATACCGGCGACGGGCGCACTTAC |
| | | TATGCAGACTCCGTCAAAGGGCGGTTTACAATCTCTAGGGACAACGCCAAGAAT |
| | | ACCGTCGATCTTCAGATGTCTTCCCTGAAGCCGGAGGATACCGCCATGTATTAC |
| | | TGTGCGGCCAGGGCTGCCCCTCTCTACTCCTCTGGATCACCTCTGACTAGGGCT |
| | | CGCTATAACGTATGGGGTCAGGGAACACAGGTCACGGTGTCATCT |
| hIL27 | 1465 | CAGGTTCAGCTCCAAGAGAGCGGCGGTGGCTCAGTGCAGGCAGGTGGCTCACTT |
| Ra_VH | | AGGCTGAGTTGCAGAGCTTCCGGCTCAACATACTCCAACTATTGTCTCGGCTGG |
| H19- | | TTCAGGCAGATCACTGGTAAGGAGCGCGAGGGGGTGGCCGTCATCAACTGGGTG |
| DR594 | | GGTGGAATGGTGTACTTTGCCGACTCTGTAAAGGGTCGTTTTACTGTGTCTCAG |
| | | GACCAGGCCAAGAACACAGTGTACCTTCAGATGAACTCTCTGAAGCCGGAGGAT |
| | | ACGGCAATGTACTATTGCGCTGCCGAGAGTGTTAGTTCATTCTCCTGTGGAGGC |
| | | TGGCTGACTCGCCCGGATCGTGTGCCGTACTGGGGCCAGGGCACCCAGGTGACC |
| | | GTTAGCTCCGGCGGTGGCTCCCAGGTGCAGCTGCAAGAGAGTGGTGGAGGCTCC |
| | | GTGCAGGCAGGTGGCTCCCTTCGTCTTTCTTGTGTCGCCTCTGCTTCAACCTAC |
| | | TGCACATACGACATGCACTGGTATCGCCAGGCCCCTGGCAAGGGGCGCGAGTTC |
| | | GTTAGTGCCATCGACAGTGACGGCACTACCAGATACGCCGACAGCGTCAAGGGC |
| | | CGCTTCACCATCAGCCAGGGAACAGCTAAAAACACCGTCTACCTCCAGATGAAC |
| | | TCCTTGCAGCCAGAGGACACAGCCATGTATTACTGCAAAACTGTCTGCGTCGTG |
| | | GGGTCCAGATGGAGCGACTACTGGGGCCAAGGCACGCAGGTTACCGTGAGCAGC |
| hIL27 | 1466 | CAGGTCCAGTTGCAGGAGTCCGGCGGGGGTTCTGTGCAGGCTGGAGGCAGCCTC |
| Ra_VH | | AGACTCTCTTGCAGGGCTTCTGGATCAACTTATTCTAATTATTGCCTCGGTTGG |
| H19- | | TTCCGCCAAATCACAGGCAAGGAGAGGGAGGGAGTGGCGGTGATTAACTGGGTG |
| DR594 | | GGCGGTATGCTGTACTTCGCCGACTCCGTGAAAGGTCGTTTCACTGTCTCTCAG |
| | | GATCAGGCCAAGAACACTGTTTACCTCCAGATGAACTCTCTGAAGCCCGAAGAT |
| | | ACGGCCATGTACTATTGCGCTGCCGAATCCGTCTCCTCTTTTTCCTGCGGAGGC |
| | | TGGCTGACACGCCCCGACCGCGTCCCCTATTGGGGTCAGGGGACTCAGGTCACC |
| | | GTCAGCTCTGGCGGGAGCGGAGGCAGCGGGGGCAGCGGCCAGGTCCAGCTTCAG |
| | | GAAAGCGGAGGTGGCTCCGTGCAGGCTGGCGGAAGCCTGAGACTGTCCTGCGTG |
| | | GCTTCAGCTTCCACCTACTGCACCTATGACATGCACTGGTATAGGCAAGGGCCA |
| | | GGAAAGGGCAGGGAGTTCGTTTCTGCTATTGATTCTGACGGAACAACTAGATAC |
| | | GCAGACTCCGTCAAGGGAAGGTTCACCATCCCCCAGGGCACCGCCAAGAACACT |
| | | GTCTACCTCCAGATGAACAGTCTGCAACCAGAAGACACCGCTATGTATTACTGC |
| | | AAGACCGTGTGCGTGGTCGGGTCCAGATGGTCCGATTACTGGGGCCAGGGAACA |
| | | CAGGTGACTGTCTCATCT |
| hIL27 | 1467 | CAGGTGCAGGTCCAGGAATCTGGGGGAGGTTCAGTGCAGGCAGGTGGCTCCCTG |
| Ra_VH | | CGCCTGAGTTGCAGGGCGTCCGGTAGTACCTACTCAAACTACTGCCTGCGGGGG |
| H19- | | TTTAGACAAATCACCGGAAAGGAAAGGGAGGGCGTCGCAGTGATAAATTGGGTG |
| DR595 | | GGCGGTATGCTGTACTTCGCCGACTCTGTGAAGGGACGGTTTACCGTCTCCCAG |
| | | GATCAGGCTAAGAACACTGTGTATTTGCAGATGAACTCCCTGAAACCAGAGGAC |
| | | ACAGCCATGTATTACTGTGCTGCCGAAAGCGTCTCTAGTTTTTCCTGCGGAGGG |
| | | TGGCTTACCCGTCCCGACAGAGTTCCTTATTGGGGCCAGGGAACCCAGGTGACA |
| | | GTTTCCTCTGGTGGGGGGTCTCAGGTTCAGCTTCAGGAGTCTGGGGGGGGCTCT |
| | | GTGCAGGCTGGCGGATCTCTGACTCTGAGCTGCGCCGCGAGCGAGTACGCCTAC |
| | | TCAACCTGCAACATGGGCTGGTATCGCCAGGCACCGGGCAAGGAACGGGAGCTG |
| | | GTGTCTGCATTCATCTCAGACGGTTCTACGTATTACGCCGACTCCGTGAAGGGT |
| | | CGCTTTACTATTACCCGCGACAACGCTAAGAACACGGTGTACTTGCAGATGAAC |
| | | AGCCTGAAGCCTGAGGATACTGCCATCTATTACTGTTCCGCTAACTGCTATAGA |
| | | CGCCTGCGCAATTACTGGGGACAGGGGACGCAAGTGACAGTGTCTAGC |
| hIL27 | 1468 | CAAGTGCAGTTGCAGGAATCCGGCGGAGGCTCCGTACAGGCCGGTGGATCACTG |
| Ra_VH | | CGCCTGTCTTGTCGGGCCTCCGGGTCAACCTATAGCAACTACTGTCTGGGCTGG |
| H19- | | TTTCGTCAGATCACAGGTAAAGAGCGCGAGGGGGTCGCCGTCATCAACTGGGTG |
| DR595 | | GGCGGTATGCTGTACTTCGCTGACAGTGTGAAGGGTCGGTTCACCGTTTCCCAG |
| | | GATCAAGCCAAAAACACCGTTCTACCTCCAGATGAACTCGTGAAGCCCGAAGAC |
| | | ACCGCCATGTATTACTGCGCCGCAGAGAGTGTGTCCTCTTTCTCGTGCGGTGGG |
| | | TGGTTGACTAGGCCGGACCGTGTTCCCTACTGGGGCCAGGGAACCCAAGTGACC |
| | | GTTAGCTCAGGGGGCTCCGGTGGCTCTGGTGGCTCTGGTCAGGTGCAGCTCCAG |
| | | GAGTGTGGAGGGGGGTCAGTTCAGGCTGGGGGCAGTCTGACTCTGTCCTGTGCT |
| | | GCAAGCGAGTACGCTTACTCTACCTGTAACATGGGTTGGTATCGCCAGGCCCCA |
| | | GGGAAGGAACGCGAGCTTGTGTCAGCCTTTATCTCCGATGGCAGCACTTATTAC |
| | | GCAGATTCCGTAAAGGGACGCTTCACCATCACCCGTGAGAACGCCAAGAATACC |
| | | GTCTACCTCCAGATGAACAGCCTGAAGCCGGAGGACACCGCCATCTATTACTGC |
| | | TCCGCCAACTGCTACCGGCGTCTGCGCAACTACTGGGGCCAGGGCACCCAGGTG |
| | | ACCGTTTCATCA |
| hIL27 | 1469 | CAGGTTCAACTCCAGGAGTCCGGCGGAGGGTCCGTGCAGGCTGGAGGTTCCCTC |
| Ra_VH | | CGGTTGTCCTGTCGTGCGTCCGGTTCCACCTACTCAAATTATTGTCTGGGCTGG |
| H19- | | TTCCGCCAGATTACTGGCAAGGAAAGAGAGGGCGTAGCCGTCATCAACTGGGTT |
| DR596 | | GGGGGAATGTTGTACTTCGCCGACAGCGTGAAAGGCCGGTTTACGGTTTCCCAG |
| | | GACCAGGCTAAGAACACTGTATACCTTCAGATGAACTCCCTGAAGCCGGAGGAC |
| | | ACCGCTATGTACTATTGCGCCGCTGAGTCTTGTCTCCAGCTCTCTTGCGGGGGA |
| | | TGGTTGACCCGCCCTGATCGCGTGCCATACTGGGGGCAGGGTACTCAAGTGACA |
| | | GTGTCCTCTGGCGGAGGCTCCCAGGTTCAGTTGCAGGAGAGCGGCGGTGGACTG |
| | | GTTCAGCCTGGTGGGAGTCTCCGCCTGAGCTGCACAGCCAGCGGACTGACTTTC |
| | | GATGACTCAGTGATGGGATGGTTCAGACAGGCTCCGGGAAAGGGCCGTGAGGCG |
| | | GTCAGCTGTATCTCCTCTTCCGGCGCTAACGCCTTCTACGCTGATTCCGTAAAG |
| | | GGACGGTTTACCATTTCTCGGGATAACGCCAAGAATACCCTGTATCTCCAGATG |
| | | AACTCTCTCAAGCCCGAGGATACCGCCACCTATTACTGTAAAAGGGGCCACGCA |
| | | TGTGCTGGATATTACCCGATTCCCTATGATGACTATTGGGGGCAAGGAACGCAG |
| | | GTTACCGTTAGTTCT |
| hIL27 | 1470 | CAGGTGCAGCTCCAGGAGTCCGGTGGGGGTTCCGTCCAGGCAGGTGGCTCCCTG |
| Ra_VH | | CGGCTGTCTTGTAGGGCTTCTGGCTCTACCTACTCCAACTACTGTCTGGGATGG |
| H19- | | TTCCGCCAGATCACAGGCAAGGAGAGGGAGGGCGTAGCAGTGATTAACTGGGTT |
| DR596 | | GGGGGTATGCTGTATTTCGCCGATTCCGTGAAGGGTCGCTTCACCGTGTCCCAG |
| | | GATCAGGCCAAAAACACTGTTTATTTGCAGATGAACTCCCTGAAGCCTGAGGAC |
| | | ACCGCTATGTATTACTGTGCAGCGGAGTCCGTGTCATCCTTCTCCTGTGGTGGC |
| | | TGGCTGACTCGTCCAGACCGTGTGCCTTATTGGGGACAGGGCACCCAGGTGACC |
| | | GTGTCCTCAGGCGGTTCCGGTGGCTCCGGTGGCAGCGGTCAGGTGCAGCTTCAG |
| | | GAGAGCGGTGGGGGCCTGGTGCAGCCCGGAGGTTCACTGAGACTGAGCTGCACG |
| | | GCCAGCGGTCTGACCTTCGATGACAGCGTGATGGGTTGGTTCCGTCAAGCGCCG |
| | | GGCAAGGGACGCGAAGCAGTGTCCTGTATCAGTTCCCCTGGTGCTAACGCCTTC |
| | | TATGCTGACAGTGTGAAGGGACGCTTTACTATCTCCCGCGACAACGCCAAGAAT |
| | | ACCCTGTACCTCCAGATGAACTCTCAGAAGCCCGAGGATACCGCCACGTATTAC |
| | | TGTAAGCGCGGTCACGCCTGTGCAGGGTACTATCCCATCCCCTACGATGACTAC |
| | | TGGGGCCAGGGAACTCAGGTCACGGTCTCCTCA |
| hIL27 | 1471 | CAGGTTCAGCTCCAGGAGAGTGGGGGGGGGCTGGTGCAGCCCGGCGGATCACTC |
| Ra_VH | | CGGCTGTCCTGTGCCGCGAGTGGGTTTACGTTTTCCTCATACCCTATGTCCTGG |
| H20- | | GTCCGCCAGGCTCCGGGCAAAGGACTGGAATGGGTAAGCACAATTAGCTCTGGG |
| DR591 | | GGCGATACAACTCTGTATGCGGACTCTGTAAAAGGACGCTTCACCAGCTCTCGC |
| | | GACAACGCAAAGAATACACTCTACCTCCAGCTTAACCCTCTGAAAACAGAGGAC |
| | | ACAGCAATGTATTACTGTGCAAAGCGCATTGATTGCAATTCCGGCTACTGCTAC |
| | | AAGCGCTCTTATTGGGGGCAAGGTACGCAGGTGACGGTTAGCTCCGGCGGAGGC |
| | | TCCCAGGTGCAGTTGCAGGAATCCGGCGGCAGGCTCAGTGCAAGGGGAGGGTCC |
| | | CTGAGACTGAGCTGTACTGCATCCGGGGCTATCGCCAGCGGATACATTGATTCA |
| | | CGGTGGTGTATGGCTTGGTTCCGGCAAGCCCCTGGCAAGGAGCGCGAGGGCGTG |
| | | GCGGCTATCTGGCCCGGCGGTGGACTGACCGTGTACGCTGACTCCGTCAAGGGC |
| | | CGCTTTACTATCAGCCGCGACCACGCTAAGAACACGCTGTACCTCCAGATGAAC |
| | | AATCTGAAGCCCGAGGACACCGCTATGTACTATTGCGCAGCTGGCTCACCTCGG |
| | | ATGTGCCCTTCCCTTGAGTTTGGGTTCGATTACTGGGGCCAAGGCACCCAGGTG |
| | | ACCGTTAGCAGT |
| hIL27 | 1472 | CAGGTGCAGGTGCAAGAATCCGGCGGTGGCCTGGTGCAGCCAGGAGGCTCCCTG |
| Ra_VH | | CGGCTGTCCTGTGCTGCGTCTGGCTTCACCTTTAGCTCTTATCCCATGTCTTGG |
| H20- | | GTGCGTCAGGCTCCGGGAAAGGGCTTGGAATGGGTGTCCACCATTTCTAGCGGA |
| DR591 | | GGCGACACCACTCTCTACGCTGACTCCGTGAAAGGGCGGTTCACCTCCAGCAGA |
| | | GACAACGCTAAGAACACCCTGTACTTGCAGCTTAATTCTCTGAAGACCGAGGAT |
| | | ACAGCAATGTACTATTGCGCCAAGCGGATTGATTGCAACTCCGGCTATTGTTAC |
| | | AAGAGGTCCTATTGGGGCCAGGGAACTCAGGTAACTGTTAGTTCAGGAGGCTCC |
| | | GGTGGATCTGGCGGAAGCGGACAGGTGCAGCTTCAGGAATCTGGCGGTGGAAGC |
| | | GTCCAGGCTGGAGGTTCCCTGAGGCTCAGCTGCACCGCCTCTGGTGCCATTGCG |
| | | TCCGGGTACATTGATAGCCGCTGGTGCATGGCCTGGTTTAGACAAGCGCCAGGC |
| | | AAGGAAAGAGAGGGTGTGGCAGCCATCTGGCCGGGGGGAGGTCTGACTGTGTAC |
| | | GCCGACAGCGTGAAGGGCCGTTTCACTATCCCTCGCGACCACGCCAAGAACACT |
| | | TTGTATCTTCAGATGAACAATCTGAAGCCCGAAGATACAGCCATGTATTACTGC |
| | | GCAGCCGGTAGCCCTAGAATGTGCCCCAGTCTGGAGTTCGGCTTCGATTATTGG |
| | | GGTCAGGGTACTCAGGTCACCGTCAGCTCT |
| hIL27 | 1473 | CAGGTGCAGGTTCAAGAATCTGGAGGGGGCCTGGTTCAGCCAGGGGGTTCTCTG |
| Ra_VH | | CGTCTGAGCTGTGCCGCGTCTGGATTCACATTCTCATCCTATCCAATGAGCTGG |
| H20- | | GTGCGTCAGGCTCCCGGTAAAGGGCTGGAATGGGTGTCTACTATCTCCAGCGGA |
| DR592 | | GGCGACACTACCCTGTATGCGGACAGCGTCAAGGGACGGTTCACGTCCAGCAGA |
| | | GACAACGCCAAAAATACCTTGTACTTGCAGCTGAACAGCCTGAAGACGGAGGAT |
| | | ACAGCTATGTACTATTGCGCTAAAAGAATCGACTGCAACTCCGGCTACTGTTAC |
| | | AAGAGAAGCTATTGGGGCCAGGGTACTCAGGTCACCGTGTCTAGCGGAGGTGGC |
| | | TCCCAAGTGCAGTTGCAGGAGTCTGGGGGGGGTTCCGTCCAGGCTGGTGGCTCC |
| | | TTGCGTCTTTCCTGTACTGCGCCGGGGTTTACCAGCAACTCATGGGGCATGGAT |
| | | TGGTATCGTCAAGCGCCTGGTAAGGAGAGGGAATTTGTGTCATCCATCTCCACC |
| | | GACGGCACGACCGGCTATGCTGACAGCGTTAAAGGTCGTTTTACCATTTCTAAG |
| | | GATAAGGCCAAGGATACCGTTCTACCTCCAGATGAACTCTCGAAGCCTGAGGAT |
| | | ACTGGCATGTACTCCTGCAAGACTAAGGATGGCACCATTGCCACGATGGAACTG |
| | | TGCGATTTCGGGTACTGGGGTCAAGGCACCCAGGTTACCGTCTCCAGC |
| hIL27 | 1474 | CAGGTCCAGCTTCAGGAAAGTGGCGGAGGCCTTGTCCAACCTGGGGGTTCACTC |
| Ra_VH | | AGACTGTCTTGCGCTGCCAGTGGTTTCACCTTTTCTTCCTACCCCATGTCATGG |
| H20- | | GTGCGCCAGGCCCCAGGGAAGGGATTGGAATGGGTCTCCACAATCTCCTGTGGC |
| DR592 | | GGTGATACCACACTGTACGCTGACTCTGTCAAGGGACGCTTCACTTCCAGCCGT |
| | | GATAACGCCAAGAACACCCTGTACTTGCAACTGAACAGTTTGAAAACCGAGGAT |
| | | ACCGCTATGTATTACTGCGCCAAGCGCATTGATTGCAACTCCGGGTATTGTTAC |
| | | AAGCGCAGCTATTGGGGCCAGGGCACCCAGGTGACTGTCTCCTCTGGTGGCTCA |
| | | GGCGGAAGCGGAGGCTCAGGGCAAGTACAGCTCCAGCGAGTCAGGTGGGGTTCC |
| | | GTACAAGCCGGGGGCAGCCTGCGTCTTAGTTGCACCGCACCAGGGTTCACCTCT |
| | | AACAGCTGCGGAATGGATTGGTACAGGCAAGCGCCCGGCAAGGAACGCGAGTTT |
| | | GTAAGCTCCATCTCCACCGATGGCACAACCGGGTACGCAGACTCTGTGAAGGGG |
| | | CGCTTCACGATCAGCAAGGACAAAGCGAAGGATACTGTGTACCTGCAAATGAAC |
| | | TCCCTCAAGCCCGAAGACACCGGCATGTATTCCTGTAAGACCAAGGACGGCACC |
| | | ATCGCCACTATGGAGCTGTGCGATTTTGGATACTGGGGACAGGGCACCCAGGTG |
| | | ACTGTCTCCTCC |
| hIL27 | 1475 | CAAGTACAGTTGCAGGAGAGCGGTGGGGGACTGGTACAGCCAGGCGGTTCCCTG |
| Ra_VH | | CGCTTGAGCTGTGCTGCCTCCGGCTTCACCTTCTCCAGCTACCCTATGTCATGG |
| H20- | | GTGCGCCAGGCCCCTGGCAAAGGTCTGGAATGGGTCTCTACCATCTCATCTGGA |
| DR593 | | GGGGACACAACCCTTTATGCAGACAGCGTCAAAGGACGTTTCACTTCTTCACGG |
| | | GACAACGCGAAGAACACCCTGTACCCTCAGCTGAACTCCCTGAAGACTGAGGAT |
| | | ACCGCAATGTACTATTGCGCCAAGCGGATTGATTGTAATTCCGGCTATTGTTAT |
| | | AAGCGCTCCTATTGGGGGCAGGGGACCCAGGTAACTGTTTCCTCAGGAGGGGGT |
| | | TCCCAGGTTCAGCTCCAGGAGTCTGGTGGAGGCTCCGTCCAAGCCGGGGGCTCA |
| | | CTGCGCCTGTCATGCGCAGCCAGCGGTTACCCCTATAGCAACGGTTATATGGGT |
| | | TGGTTCAGGCAGGCCCCCGGTAAAGAACGCGAGGGCGTCGCTACCATCTACACG |
| | | GGCGACGGCAGGACTTATTACGCCGACTCTGTCAAGGGGCGTTTTACTATCTCT |
| | | CGCGACAATGCCAAGAACACGGTGGACCTTCAAATGTCCAGTCTGAAGCCGGAA |
| | | GACACTGCCATGTATTACTGCGCAGCGCGTGCGGCCCCTCTGTATTCCTCAGGC |
| | | AGCCCTCTGACCCGCGCTCGCTACAACGTGTGGGGCCAAGGCACGCAAGTAACT |
| | | GTGTCCTCT |
| hIL27 | 1476 | CAGGTGCAGCTCCAGGAGTCTGGCGGTGGCCTGGTACAGCCGGGAGGCTCCCTG |
| Ra_VH | | CGCCTGAGCTGTGCAGCTTCTGGCTTCACCTTCTCTTCCTACCCAATGTCCTGG |
| H20- | | GTGCGTCAGGCTCCCGGTAAGGGCCTTGAGTGGGTGTCTACCATTTCTTCCGGG |
| DR593 | | GGTGATACCACGTTGTACGCAGACTCCGTGAAGGGCAGGTTCACCAGCTCTCGG |
| | | GACAATGCCAAGAACACTCTGTACTTGCAGCTGAACTCTTTGAAGACCGAGGAT |
| | | ACCGCAATGTACTATTGTGCAAAGAGGATCGACTGTAACTCTGGCTATTGTTAT |
| | | AAGAGAAGCTACTGGGGGCAGGGTACTCAGGTGACCGTGTCAAGCGGGGGAAGC |
| | | GGTGGGTCTGGAGGGAGTGGGCAGGTGCAGCTCCAGGAGTCTGGAGGGGGGAGC |
| | | GTGCAGGCAGGCGGTAGCCTCCGGCTGTCATGCGCCGCTTCCGGTTATCCCTAT |
| | | AGCAACGGATACATGGGATGGTTCCGTCAGGGTCCCGGCAAGGAAAGGGAGGGT |
| | | GTGGCCACGATCTACACAGGGGATGGGAGAACTTATTACGCCGACTCCGTTAAG |
| | | GGACGCTTCACTATCAGCCGCGACAACGCTAAAAACACTGTGGACCTTCAGATG |
| | | TCCAGCTTGAAGCCTGAGGATACCGCCATGTATTACTGTGCAGCTCGCGCCGCT |
| | | CCCCTGTACTCAAGCGGTTCACCCCTCACCCGCGCTCGCTACAACGTCTGGGGG |
| | | CAGGGCACACAGGTGACTGTGTCCTCT |
| hIL27 | 1477 | CAGGTGCAGTTGCAAGAGAGGGGCGGGGGCCTGGTCCAGCCTGGGGGCTCACTG |
| Ra_VH | | CGTCTCTCCTGCGCCGCATCTGGATTTACCTTGTCCTCTTATCCTATGAGTTGG |
| H20- | | GTACGCCAGGCTCCTGGCAAGGGTTTGGAGTGGGTTTCCACAATCTCTAGCGGA |
| DR594 | | GGTGACACTACACTTTACGCTGATAGCGTCAAGGGACGCTTTACCTCATCCAGG |
| | | GACAACGCTAAGAACACCCTCTATCTCCAGCTGAACTCCCTCAAGACTGAGGAT |
| | | ACAGCCATGTACTATTGCGCTAAACGCATCGACTGTAACAGCGGATACTGCTAC |
| | | AAGCGTAGCTACTGGGGCCAGGGCACCCAGGTTACCGTCAGCTCTGGAGGTGGG |
| | | TCCCAGGTCCAGTTGCAGGAAAGTGGCGGTGGGTCAGTGCAAGCTGGGGGATCT |
| | | CTGCGCCTGTCCTGTGTGGCCAGCGCGTCTACCTACTGTACCTACGATATGCAC |
| | | TGGTACAGACAAGCGCCTGGTAAAGGTAGGGAGTTTGTGAGCGCTATCGACTCC |
| | | GATGGAACCACGAGGTATGCAGACTCCGTCAAGGGAAGGTTCACTATTTCCCAG |
| | | GGCACAGCCAAAAACACCGTCTACTTGCAGATGAACAGCTTGCAGCCCGAGGAC |
| | | ACCGCGATGTACTATTGCAAAACGGTGTGCGTAGTGGGGTCCCGGTGGAGCGAC |
| | | TACTGGGGCCAGGGAACTCAGGTAACAGTCTCGCC |
| hIL27 | 1478 | CAGGTCCAGGTGCAAGAGTCTGGGGGGGGACTGGTTCAGCCTGGGGGCAGCCTG |
| Ra_VH | | CGGCTCAGCTGTGCAGCCTCCGGCTTCACTTTTTCCAGCTACCCTATGTCTTGG |
| H20- | | GTGCGCCAGGCTCCCGGTAAGGGCCTGGAGTGGGTGTCCACGATTTCTAGTGGG |
| DR594 | | GGTGATACGACCCTGTATGCCGACAGTGTAAAGGGTCGGTTCACAAGCAGTCGT |
| | | GACAACGCTAAAAACACCCTGTACCTTCAGCTCAATTCCCTGAAAACAGAGGAC |
| | | ACTGCTATGTATTACTGCGCGAAGCGTATCGACTGTAACAGCGGGTATTGCTAT |
| | | AAGCGCAGTTATTGGGGCCAGGGCACGCAGGTCACCGTGAGTTCGGGGGGTTCT |
| | | GGTGGCAGCGGCGGTTCTGGGCAGGTTCAGCTTCAGGAGAGTGGTGGGGGGTCC |
| | | GTTCAGGCCGGGGGCTCTTTGCGGCTGTCATGCGTGGCGTCCGCTTCTACCTAC |
| | | TGCACCTATGATATGCACTGGTATCGCCAAGCGCCCGGCAAGGGTCGCGAGTTT |
| | | GTCAGCGCCATCGACTCCGACGGCACGACTCGGTACGCCGACTCTGTCAAGGGG |
| | | CGGTTCACGATCTCCCAGGGCACCGCCAAGAACACAGTGTATGTTCAGATGAAC |
| | | TCTCTGCAACCTGAAGATACTGCAATGTACTATTGTAAGACCGTGTGCGTGGTC |
| | | GGCTCCAGATGGAGCGACTACTGGGGTCAGGGCACCCAGGTGACTGTGAGTAGT |
| hIL27 | 1479 | CAGGTGCAACTGCAAGAGTCTGGAGGCGGTCTGGTGCAGCCAGGAGGTAGTCTT |
| Ra_VH | | CGTCTGTCTTGCGCGGCCTCCGGCTTTACATTCTCCAGCTACCCAATGTCCTGG |
| H20- | | GTGCGTCAGGCCCCCGGCAAGGGCCTGGAGTGGGTTTCTACCATCTCTAGTGGC |
| DR595 | | GGTGACACAACCCTGTACGCTGACTCTGTTAAAGGAAGATTTACCTCCAGTCGG |
| | | GACAACGCCAAGAACACACTTTACTTGCAACTGAACTCTCTGAAAACCGAGGAC |
| | | ACCGCCATGTATTACTGCGCTAAGCGTATTGACTGCAATTCAGGCTACCGTTAT |
| | | AAGCGCTCTTACTGGGGCCAGGGCACTCAGGTCACCGTGTCTTCAGGGGGTGGC |
| | | TCTCAGGTGCAGCTCCAGGAAAGCGGTGGGGGCTCCGTCCAAGCCGGTGGCAGC |
| | | CTGACACTTTCCTGTGCTGCCTCCGAATACGCTTACTCAACCTGTAACATGGGC |
| | | TGGTACAGACAGGCTCCTGGCAAGGAAAGAGAGTTGGTGTCCGCTTTCATCTCC |
| | | GACGGCTCCACTTATTACGCCGACTCTGTAAAGGGCAGGTTCACGATCACCCGC |
| | | GACAATGCCAAAAACACCGTCTACTTGCAGATGAACAGGCTGAAGCCGGAAGAC |
| | | ACAGCAATCTACTATTGTAGCGCGAACTGCTATCGGCGTCTGCGCAACTATTGG |
| | | GGCCAGGGCACCCAGGTCACCGTCTCCTCC |
| hIL27 | 1480 | CAGGTGCAGCTCCAGGAATCTGGGGGCGGTCTGGTTCAGCCGGGGGGTAGTCTG |
| Ra_VH | | CGGCTCTCCTGTGCCGCAAGCGGTTTTACTTTGTCTAGCTATCCCATGAGTTGG |
| H20- | | GTTCGGCAGGCCCCAGGTAAAGGGTTGGAGTGGGTATCTACCATCAGCTCTGGA |
| DR595 | | GGTGACACTACCCTGTACGCCGACTCTGTGAAGGGCCGTTTCACCTCTTCACGC |
| | | GACAACGCAAAAAATACGCTGTACCTCCAGCTCAATTCACTGAAAACTGAGGAC |
| | | ACCGCTATGTATTACTGTGCGAAGCGCATTGATTGTAACAGCGGATATTGCTAC |
| | | AAGCGCTCCTACTGGGGACAAGGTACGCAGGTGACTGTCTCCAGCGGCGGTAGC |
| | | GGGGGTTCTGGAGGTTCCGGCCAGGTGCAACTGCAAGAGTCCGGGGGTGGCTCC |
| | | GTGCAGGCCGGTGGGTCCCTGACTCTGTCCTGTGCTGCATCAGAATATGCGTAT |
| | | TCCACTTGCAACATGGGTTGGTACAGGCAGGCTCCCGGAAAAGAGCGCGAGCTG |
| | | GTCTCCGCCTTCATCAGTGACGGGTCTACCTATTACGCGGACAGTGTGAAGGGC |
| | | CGCTTCACCATCACACGTGATAACGCCAAGAATACCGTGTATTTGCAAATGAAC |
| | | TCTCTGAAACCCGAGGACACCGCAATCTATTACTGTAGCGCTAACTGCTACCGC |
| | | CGTCTCAGGAACTATTGGGGACAGGGCACGCAGGTCACAGTTTCGTCC |
| hIL27 | 1481 | CAGGTACAGGTTCAAGAGTCTGGGGGAGGTTTGGTGCAGCCTGGTGGGTCCTTG |
| Ra_VH | | CGGCTGTCCTGTGCCGCGAGCGGGTTTACGTTGTCTTCCTATCCTATGTCTTGG |
| H20- | | GTGAGGCAAGCGCCGGGCAAGGGCCTTGAATGGGTGTCCACCATCTCTAGCGGA |
| DR596 | | GGCGACACGACCCTTTACGCTGACTCTGTGAAGGGACGGTTTACATCAAGTAGA |
| | | GACAACGCCAAGAATACACTGTATTTGCAGCTGAACTCCCTGAAGACTGAGGAC |
| | | ACTGCCATGTATTACTGCGCTAAGAGAATTGACTGCAACTCAGGCTACTGCTAC |
| | | AAGCGCAGCTACTGGGGCCAGGGAACTCAGGTGACCGTTAGCTCGGGTGGCGGA |
| | | TCTCAGGTACAGTTGCAAGAGTCAGGAGGTGGCCCTGGTGCAGCCCGGGGTAGC |
| | | CTGCGTCTTAGCTGTACTGCCAGCGGCTTGACATTCGATGACTCCGTGATGGGT |
| | | TGGTTTAGACAGGCCCCTGGCAAAGGACGGGAGGCTGTGAGCTGTATCAGCTCC |
| | | TCAGGAGCTAATGCTTTCTACGCCGACTCCGTCAAGGGACGTTTTACAATCTCA |
| | | CGTGATAACGCTAAGAATACTCTGTATTTGCAAATGAACTCTCTGAAGCCAGAG |
| | | GATACCGCTACCTATTACTGCAAGAGAGGCCACGCCTGCGGGGGGTATTACCCC |
| | | ATTCCTTACGACGATTACTGGGGTCAGGGTACACAGGTGACCGTCAGTTCT |
| hIL27 | 1482 | CAGGTACAGTTGCAGGAGTCTGGAGGCGGACTGGTGCAACCCGGAGGCAGCCTG |
| Ra_VH | | AGGCTGAGCTGCGCAGCCAGCGGATTTACCTTCTCCTGTTATCCCATGAGCTGG |
| H20- | | GTTCGCCAAGCGCCAGGTAAAGGTCTGGAATGGGTCAGCACAATCAGCTCAGGC |
| DR596 | | GGTGACACTACCCTGTACGCAGACTCTGTGAAAGGCCGGTTCACCTCTTCCAGG |
| | | GATAACGCCAAGAATACGCTCTACCTCCAGCTGAACTCCCTCAAGACTGAAGAC |
| | | ACCGCGATGTACTATTGCGCAAAGAGAATTGATTGTAACAGCGGTTATTGTTAC |
| | | AAACGTAGCTACTGGGGTCAGGGCACCCAGGTCACCGTGTCTTCCGGGGGAAGT |
| | | GGAGGTTCTGGTGGCTCTGGACAGGTACAGTTGCAGGAGAGCGGCGGTGGCCTG |
| | | GTGCAGCCTGGGGGAAGCCTGCGGCTGAGCTGCACTGCGAGCGGTTTGACTTTC |
| | | GATGACTCTGTTATGGGTTGGTTCCGTCAGGCCCCTGGTAAGGGCCGTGAGGCG |
| | | GTTTCCTGCATCTCTTCATCCGGTGCTAACGCTTTTTATGCAGACTCCGTGAAG |
| | | GGTCGCTTTACAATCAGCAGGGATAACGCGAAAAACACCCTCTACCTGCAAATG |
| | | AACTCCCTGAAACCCGAAGACACTGCTACCTATTACTGCAAGAGGGGGCACGCC |
| | | TGCGCTGGCTATTACCCTATCCCTTACGATGACTACTGGGGTCAGGGAACCCAG |
| | | GTCACCGTGTCCTCC |
| hIL27 | 1483 | CAAGTGCAGTTGCAGGAAAGCGGAGGGGGACTTGTTCAGCCAGGGGGATCTTTG |
| Ra_VH | | CGCCTCAGCTGTGCCGCTTCAGGGTTTACATTCTCCCTGTCTAGCATGAGCTGG |
| H21- | | GTCCGTCAGGCTCCTGGAAAGGGTCTGGAATGGGTTTCCGCCATTAGCTCCGGC |
| DR591 | | GGTGCCTCCACTTATTACACCGACAGTGTGAAAGGCCGCTTTACAATCAGCCGC |
| | | GACAACGCCAAGAACATGCTGTACTTGCAACTGAACAGCCTGAAAACCGAGGAC |
| | | ACCGCCATGTACTATTGTGCCAAGGGGGGTCCGGCTACCGGCGACGCGAGCCGG |
| | | ATGACCTCCCCAGGCTCCCAGGGCACCCAGGTCACCGTGAGCAGTGGGGGAGGG |
| | | TCCCAGGTTCAGTTGCAGGAGTCTGGAGGGGGCTCCGTGCAGGCTGGCGGAAGC |
| | | CTGCGCCTGAGCTGCACTGCCTCCGGCGCTATCGCTTCAGGATATATTGATTCC |
| | | CGTTGGTGTATGGCCTGGTTTAGGCAGGCTCCTGGAAAGGAGCGCGAAGGCGTG |
| | | GCAGCTATCTGGCCTGGCGGTGGCCTGACCGTATACGCAGATAGTGTCAAGGGC |
| | | CGCTTCACTATCTCCCGCGACCACGCAAAGAACACGCTGTACCTCCAGATGAAT |
| | | AACCTGAAGCCTGAGGACACCGCCATGTATTACTGTGCCGCTGGCTCACCTCGT |
| | | ATGTGCCCCTCCCTGGAGTTCGGCTTTGACTACTGGGGCCAGGGAACCCAGGTC |
| | | ACCGTCTCTTCT |
| hIL27 | 1484 | CAGGTGCAGCTTCAGGAGAGCGGAGGGGCCTGGTTCAGCCTGGTGCGGTCACTC |
| Ra_VH | | CGCCTGAGTTGTGCGGCCTCAGGATTTACATTCTCCCTCTCCAGCATGTCTTGG |
| H21- | | GTTAGACAAGCTCCTGGCAAAGGTCTGGAGTGGGTATCCGCCATTAGCTCCGGT |
| DR591 | | GGCGCTTCTACTTACTATACCGACAGCGTGAAGGGTCGCTTCACTATCTCCCGC |
| | | GACAATGCCAAGAACATGCTGTACCTTCAGCTCAATTCTCTGAAGACCGAGGAC |
| | | ACTGCGATGTACTATTGCGCCAAGGGAGGCTCTGGTTATGGCGACGCATCTCGC |
| | | ATGACCTCCCCCGGTTCCCAAGGCACCCAGGTCACGGTGTCCAGCGGAGGCAGT |
| | | GGCGGTTCAGGCGGGAGCGGACAGGTGCAGCTCCAGGAGTCCGGGGGGGGCTCA |
| | | GTGCAGGCTGGAGGCTCCCCTCGTCTGAGCTGCACTGCTTCCGGGGCAATCGCG |
| | | AGCGGCTACATTGATTCTCGTTGGTGTATGGCGTGGTTTCGTCAGGCACCCGGC |
| | | AAGGAGCGCGAAGGGGTTGCTGCAATTTGGCCGGGAGGGGCGTTGACGGTCTAT |
| | | GCGGACTCCGTGAAGGGCCGCTTCACTATCTCCCGCGACCATGCCAAGAACACA |
| | | CTGTATCTTCAGATGAATAACCTGAAGCCTGAGGACACTGCAATGTACTATTGC |
| | | GCCGCAGGCTCTCCCCGTATGTGCCCGTCACTGGAATTTGGGTTTGACTATTGG |
| | | GGCCAAGGCACACAAGTAACCGTGTCCAGC |
| hIL27 | 1485 | CAGGTCCAGCTGCAAGAGTCTGGCGGTGGCCTCGTGCAGCCCGGAGGCAGCCTG |
| Ra_VH | | AGGCTCAGCTGTGCCGCAAGTGGATTTACCTTTTCACTCAGCTCTATGTCCTGG |
| H21- | | GTGCGTCAGGCTCCAGGAAAGGGACTGGAGTGGGTGTCTGCTATCTCATCTGGA |
| DR592 | | GGGGCGTCTACATATTACACTGATAGTGTGAAGGGGCGGTTCACCATCAGTCGT |
| | | GATAACGCGAAAAACATGCTGTACCTGCAACTGAACAGTCTGAAGACTGAGGAC |
| | | ACCGCCATGTACTATTGTGCGAAGGGTGGAAGTGGCTACGGTGATGCGTCAAGA |
| | | ATGACCTCTCCCGGTAGTCAGGGAACTCAGGTCACTGTGTCCAGTGGCGGAGGC |
| | | AGCCAGGTGCAGCTCCAGGAAAGTGGAGGGGGTTCTGTCCAGGCAGGGGGCTCC |
| | | CTGCGCCTGAGCTGTACCGCCCCCGGCTTCACCTCCAACTCCTGTGGAATGGAT |
| | | TGGTACAGACAGGCTCCCGGCAAGGAGAGGGAGTTCGTTAGCTCCATCTCTACA |
| | | GACGGAACCACTGGGTACGCCGATAGCGTGAAGGGGCGTTTTACCATCTCCAAA |
| | | GATAAAGCCAAGGATACTGTGTATTTGCAGATGAACTCCCTCAAGCCCGAGGAT |
| | | ACCGGAATGTATAGCTGCAAAACCAAGGACGGTACTATCGCTACGATGGAGCTG |
| | | TGCGACTTTGGCTACTGGGGCCAAGGCACCCAGGTGACCGTTTCTAGC |
| hIL27 | 1486 | CAGGTCCAGTTGCAGGAGTCCGGCGGGGGCCTGGTTCAGCCGGGGCGCTCTCTG |
| Ra_VH | | CGCCTGAGCTGCGCCGCTTCTGGCTTCACTTTCTCTCTCAGCTCTATGTCCTGG |
| H21- | | GTCAGACAAGCGCCGGGCAAAGGTTTGGAGTGGGTGTCTGCTATTTCATCCGGT |
| DR592 | | GGCGCGAGTACCTACTATACCGATTCCGTGAAGGGCCGGTTCACTATTAGCCGG |
| | | GACAATGCTAAGAACATGCTGTACCTTCAGCTCAATTCTCTGAAAACCGAGGAC |
| | | ACTGCCATGTATTACTGCGCTAAGGGAGGTTCAGGATACGGCGATGCCTCCAGG |
| | | ATGACTAGCCCAGGCAGCCAAGGGACACAGGTCACCGTTTCCAGTGGGGGCTCC |
| | | GGCGGTTCTGGCGGTAGCGGCCAAGTGCAGCTGCAAGAGTCCGGGGGGGGCAGC |
| | | GTTCAAGCTGGGGGTAGCCTCAGGCTTTCCTGCACAGCTCCAGGTTTCACCAGC |
| | | AACAGTTGCGGAATGGACTGGTATCGCCAAGCGCCAGGTAAGGAGAGGGAGTTT |
| | | GTTTCCAGCATCTCTACCGACGGCACCACGGGATATGCTGATTCTGTGAAGGGA |
| | | CGCTTTACCATCTCTAAGGATAAAGCGAAGGACACCGTGTACTTGCAGATGAAC |
| | | AGCCTGAAGCCCGAGGATACCGGCATGTATAGGTGCAAAACTAAGGACGGAACC |
| | | ATTGCGACAATGGAACTCTGCGATTTCGGATACTGGGGTCAGGGAACCCAGGTG |
| | | ACTGTGTCTAGC |
| hIL27 | 1487 | CAGGTCCAGCTCCAGGAATCTGGCGGAGGGCTGGTGCAGCCGGGGGGTAGCCTC |
| Ra_VH | | CGGTTGTCCTGCGCTGCCAGCGGCTTTACCTTTTCCCTCAGCTCCATGTCTTGG |
| H21- | | GTGCGCCAGGCACCCGGAAAAGGATTGGAGTGGGTGTCCGCTATCTCATCCGGG |
| DR593 | | GGAGCCAGCACGTATTACACTGATTCCGTGAAGGGCCGGTTCACCATTTCTCGC |
| | | GATAACGCTAAAAACATGCTGTATTTGCAGCTTAACTCCCTGAAAACAGAAGAT |
| | | ACCGCGATGTATTACTGTGCTAAGGGTGGGTCCGGCTACGGCGACGCATCTCGT |
| | | ATGACCAGCCCTGGCAGCCAGGGCACGCAGGTCACCGTGTCAAGCGGAGGCGGT |
| | | TCCCAGGTTCAGTTGCAGGAGAGCGGTGGCGGTAGCGTGCAGGCTGGGGGTTCC |
| | | CTTCGCCTCTCTTGCGCGGCTTCCGGTTATCCATACAGCAACGGCTATATGGGG |
| | | TGGTTCAGACAGGCACCCGGCAAGGAACGCGAAGGCGTGGCAACCATCTACACT |
| | | GGCGACGGACGCACGTACTATGCTGACAGCGTTAAAGGCCGTTTTACTATCAGC |
| | | CGCGACAATGCGAAGAACACCGTCGATTTGCAGATGTCAAGTCTGAAGCCAGAA |
| | | GACACAGCCATGTACTATTGCGCTGCGAGAGCCGCTCCCCTCTACTCTAGCGGC |
| | | AGCCCCCTGACCCGCGCACGTTACAATGTGTGGGGCCAGGGCACCCAGGTGACC |
| | | GTCTCCTCC |
| hIL27 | 1488 | CAGGTGCAGCTTCAAGAATCTGGCGGGGGCTTGGTGCAGCCCGGCGGTAGCCTC |
| Ra_VH | | CGGTTGAGCTGCGCCGCTTCAGGGTTTACCCTCAGCTTGTCATCCATGTCATGG |
| H21- | | GTGCGCCAGGCCCCCGGCAAGGGACTGGAGTGGGTCAGCGCCATCAGTAGCGGG |
| DR593 | | GGAGCGTCAACTTATTACACTGACTCCGTCAAGGGTCGCTTCACAATTAGCCGG |
| | | GACAACGCCAAGAACATGCTGTACCTCCAGTTGAACTCCCTGAAAACTGAGGAC |
| | | ACAGCCATGTACTATTGCGCCAAAGGGGGTAGCGGTTATGGCGACGCTTCCCGC |
| | | ATGACCTCTCCCGGCTCCCAAGGCACCCAGGTCACTGTAAGCTCAGGAGGGAGC |
| | | GGCGGTTCCGGTGGCAGCGGACAGGTCCAGCTCCAGGAGAGTGGAGGGGGATCA |
| | | GTGCAAGCCGGTGGCTCCCTCAGGCTCTCCTGCGCTGCGTCTGGCTACCCATAT |
| | | AGCAACGGCTACATGGGATGGTTCCGCCAGGCCCCAGGCAAAGAGCGGGAGGGC |
| | | GTTGCCACCATCTACACCGGGGATGGCCGTACCTATTACGCAGACTCCGTGAAG |
| | | GGGAGGTTTACCATCTCTCGGGACAATGCCAAAAACACGGTGGACCTCCAGATG |
| | | AGTTCCCTGAAGCCCGAAGACACCGCCATGTATTACTGGGCAGCCAGAGCCGCT |
| | | CCTTTGTACTCCTCAGGTAGCCCCCTCACCCGTGCTCGCTATAATGTATGGGGA |
| | | CAGGGCACTCAAGTGACCGTGTCCTCC |
| hIL27 | 1489 | CAGGTTCAGCTTCAGGAGTCTGGTGGGGGCCTGGTGCAGCCAGGTGGCAGCTTG |
| Ra_VH | | CGCCTGTCCTGCGCGGCCAGCGGATTTACTTTCTCTCTCAGCTCCATGTCTTGG |
| H21- | | GTGAGGCAGGCTCCAGGTAAGGGCCTGGAGTGGGTTTCTGCTATTAGCTCCGGG |
| DR594 | | GGAGCTTCCACATATTACACAGATTCTGTGAAAGGTCGTTTCACGATCCCTCGC |
| | | GACAACGCCAAAAACATGCTGTACCCTCAGCTGAACAGCCTGAAAACCGAAGAC |
| | | ACAGCTATGTACTATTGCGCCAAGGGAGGTAGCGGGTACGGCGACGCCTCCAGG |
| | | ATGACAAGTCCCGGTTCCCAGGGCACCCAGGTGACGGTCAGCTCCGGTGGAGGT |
| | | AGTCAAGTCCAACTTCAGGAGTCCGGCGGTGGCTCCGTTCAGGCTGGCGGTTCT |
| | | CTGCGTCTGTCTTGTGTGGCGTCCGCTTCCACCTACTGCACTTATGATATGCAC |
| | | TGGTACAGGCAAGCGCCCGGTAAGGGGCGCGAGTTTGTTTCCGCCATTGATTCC |
| | | GATGGGACCACTCGGTATGCGGACAGCGTCAAGGGCCGCTTCACTATCTCACAG |
| | | GGTACAGCCAAGAACACAGTGTACCTCCAGATGAACTCATTGCAACCCGAGGAC |
| | | ACGGCCATGTACTATTGTAAGACAGTCTGCGTGGTCGGTTCCAGATGGTCAGAC |
| | | TACTGGGGCCAAGGGACCCAAGTCACAGTCTCGTCA |
| hIL27 | 1490 | CAGGTGCAACTCCAGGAAAGTGGCGGTGGCCTTGTTCAGCCTGGAGGCTCCCTG |
| Ra_VH | | CGGCTTAGCTGCGCTGCCTCCGGGTTTACTTTCTCACTGTCATCTATGAGCTGG |
| H21- | | GTCCGGCAAGCTCCAGGGAAGGGCCTGGAATGGGTCAGCGCTATCTCTTCCGGC |
| DR594 | | GGAGCCTCAACTTACTATACTGACTCCGTGAAGGGCCGGTTTACCATCAGTCGC |
| | | GACAACGCTAAGAACATGCTGTACCTTCAGCTGAACAGCCTGAAGACTGAAGAT |
| | | ACTGCTATGTACTATTGCGCCAAAGGCGGTTCCGGGTACGGCGACGCCTCTCGG |
| | | ATGACCTCTCCGGGTTCCCAAGGCACCCAGGTTACCGTGTCCAGTGGGGGCTCG |
| | | GGGGGTAGCGGTGGCTCTGGCCAGGTGCAGCTCCAGGAATCCGGTGGGGGGTCC |
| | | GTGCAGGCTGGAGGGTCACTGCGCCTGTCCTGCGTGGCCAGCGCCTCTACCTAC |
| | | TGCACATACGACATGCACTGGTACAGGCAAGCTCCAGGTAAGGGTCGGGAGTTT |
| | | GTCAGCGCTATCGACAGCGACGGCACAACTCGCTATGGGGATAGCGTCAAGGGG |
| | | CGTTTTACCATCAGCCAAGGGACTGCCAAAAACACAGTGTATCTCCAGATGAAG |
| | | TCTCTCCAGCCAGAGGATACTGCCATGTATTACTGCAAGACCGTGTGCGTTGTG |
| | | GGTTCACGTTGGTCCGACTATTGGGGCCAAGGCACACAGGTGACCGTGTCCTCA |
| hIL27 | 1491 | CAGGTGCAGCTCCAGGAATCTGGCGGGGGTCTGGTGCAGCCCGGCGGGTCTTTG |
| Ra_VH | | CGCCTCTCATGTGCAGCCTCTGGGTTTACCTTCTCTCTGTCCAGCATGTCTTGG |
| H21- | | GTTAGGCAGGCCCCAGGTAAGGGGCTGGAGTGGGTTTCCGCCATGTCATCCGGG |
| DR595 | | GGTGCCAGCACATATTACACCGACTCCGTCAAGGGGAGATTCACCATCAGCCGG |
| | | GACAACGCCAAGAACATGCTGTATCTCCAGCTCAACTCCCTGAAAACCGAGGAC |
| | | ACCGCCATGTATTACTGCGCCAAGGGGGGTTCCGGCTATGGTGATGGTTCAAGG |
| | | ATGACTAGCCCCGGTTCCCAGGGCACCCAAGTCACTGTATCCAGCGGGGGAGGG |
| | | TCCCAGGTGCAGCTTCAGGAGTCTGGCGGTGGCAGCGTGCAGGCCGGGGGCTCC |
| | | CTGACACTGTCCTGTGCCGCTTCCGAGTACGCCTATTCCACGTGTAACATGGGA |
| | | TGGTATCGCCAGGCTCCTGGCAAGGAGCGCGAGCTGGTCAGCGCCTTTATCTCC |
| | | GACGGCAGCACTTATTACGCCGACTCCGTGAAGGGACGGTTCACAATCACTCGC |
| | | GACAACGCCAAGAATACTGTGTACCTCCAGATGAACAGCCTCAAGCCTGAGGAT |
| | | ACCGCCATCTATTACTGCTCTGCTAATTGCTATCGTCGGCTGCGCAACTACTGG |
| | | GGACAAGGAACACAGGTGACCGTGTCATCT |
| hIL27 | 1492 | CAGGTTCAACTCCAGGAAAGCGGAGGTGGACTTGTGCAGCCTGGCGGTTCCCTT |
| Ra_VH | | CGGCTGTCCTGCGCTGCCTCTGGCTTCACTTTGTCACTCTCATCCATGAGCTGG |
| H21- | | GTCCGCCAGGCCCCCGGCAAAGGCCTGGAGTGGGTGAGTGCTATCTCCTCTGGC |
| DR595 | | GGAGCTTCCACCTACTATACTGACTCTGTGAAGGGCCGCTTCACCATTAGTCGG |
| | | GACAACGCCAAGAACATGCTGTACTTGCAGCTGAACTCACTGAAGACCGAGGAC |
| | | ACTGCCATGTACTATTGCGCTAAGGGAGGCTCCGGGTACGGCGACGCCTCCAGA |
| | | ATGACATCTCCCGGCTCTCAGGGGACCCAGGTCACGGTGTCCAGGGGGGGGTCC |
| | | GGGGGCTCCGGCGGATCTGGTCAGGTACAACTTCAGGAGTCAGGGGGAGGCAGC |
| | | GTTCAAGCCGGAGGCTCCTTGACACTGTCCTGCGCGGCCTCTGAGTACGCATAC |
| | | AGTACCTGCAACATGGGCTGGTATCGGCAGGCCCCCGGAAAGGAGCGCGAATTG |
| | | GTTTCTGCTTTCATCAGCGATGGATCTACTTATTACGCCGACTCTGTAAAAGGT |
| | | AGATTCACTATTACTCGTGACAACGCTAAAAACACGGTGTATCTGCAAATGAAC |
| | | AGCCTGAAGCCCGAGGACACTGCCATTTATTACTGTTCCGCTAATTGTTACCGT |
| | | AGGCTGCGCAATTACTGGGGTCAGGGTACTCAGGTCACTGTATCCAGC |
| hIL27 | 1493 | CAAGTCCAACTGCAAGAGTCTGGTGGAGGCTTGGTACAGCCCGGAGGGAGCCTG |
| Ra_VH | | CGCCTCTCCTGCGCAGCCTCTGGCTTCACCTTGTCTCTCAGCTCAATGTCCTGG |
| H21- | | GTGAGGCAGGCCCCCGGAAAGGGCCTTGAGTGGGTGTCTGCTATCTCCTCTGGA |
| DR596 | | GGGGCCAGCACCTACTATACAGACAGCGTCAAGGGCAGATTTACCATTAGTCGT |
| | | GATAATGCTAAAAATATGCTCTACTTGCAGCTGAACTCCCTGAAAACAGAGGAC |
| | | ACCGCCATGTACTATTGCGCCAAGGGGGGTTCAGGATATGGAGATGCCAGTCGG |
| | | ATGACCAGCCCCGGTAGTCAGGGTACACAAGTGACCGTATCTAGCGGGGGAGGC |
| | | TCTCAGGTGCAGCTCCAGGAGTCTGGAGGGGGCTTGGTGCAGCCGGGAGGCTCT |
| | | CTCCGCCTTAGTTGTACTGCCTCTGGACTGACTTTCGATGACTCAGTGATGGGC |
| | | TGGTTCCGCCAGGCCCCAGGCAAGGGCAGAGAGGCCGTGTCTTGTATCAGCAGT |
| | | AGCGGGGCCAACGCTTTCTATGCCGACTCTGTCAAGGGCCGTTTCACCATCTCC |
| | | CGCGACAACGCTAAGAATACCCTGTACCTCCAAATGAACTCCCTGAAACCCGAG |
| | | GACACAGCAACGTATTACTGTAAGAGAGGTCATGCTTGTGGGGGGTATTACCCA |
| | | ATTCCCTATGACGATTACTGGGGCCAGGGGACCCAGGTAACAGTAAGCTCC |
| hIL27 | 1494 | CAGGTGCAGTTGCAGGAGTCCGGCGGTGGGCTGGTCCAGCCTGGGGGCAGCCTG |
| Ra_VH | | CGCCTTAGTTGCGCCGCTTCCGGCTTCACCTTTAGCCTTAGCAGTATGAGCTGG |
| H21- | | GTGAGACAGGCCCCAGGCAAGGGGCTGGAGTGGGTGTCCGCAATTTCTTCCGGG |
| DR596 | | GGTGCGAGCACCTATTACACTGATAGCGTTAAGGGACGTTTCACCATTTCTCGC |
| | | GACAACGCTAAGAACATGCTGTACCTCCAGCTCAACAGCCTGAAGACAGAAGAT |
| | | ACTGCTATGTATTACTGTGCAAAGGGAGGCAGCGGCTACGGCGATGCGAGCCGG |
| | | ATGACCAGTCCGGGGTCCCAGGGCACTCAGGTCACCGTTAGCTCCGGCGGGTCT |
| | | GGCGGTTCCGGCGGTTCCGGTCAAGTTCAGCTGCAAGAGAGCGGTGGGGGACTG |
| | | GTGCAGCCGGGAGGCTCCCTGCGCCTGAGTTGCACCGCTTCCGGTCTGACCTTT |
| | | GACGATTCCGTTATGGGCTGGTTTCGCCAAGCCCCTGGCAAGGGGAGGGAAGCT |
| | | GTGTCCTGCATTTCTAGCTCTGGCGCTAACGCCTTCTATGCCGATTCCGTGAAG |
| | | GGCCGCTTCACGATCAGCCGCGACAACGCCAAGAACACCCTGTACCTGCAAATG |
| | | AACTCATTGAAGCCCGAGGATACCGCCACCTATTACTGCAAACGCGGCCATGCT |
| | | TGCGCGGGCTATTACCCGATTCGTTACGACGATTACTGGGGTCAAGGCACCCAG |
| | | GTGACAGTCTCCTCC |
| hIL27 | 1495 | CAGGTGCAGGTCCAGGAATCCGGGGGAGGCTCTGTGCAGGCTGGTGGCAGCCTG |
| Ra_VH | | AGGCTGAGTTGCCGCGCTTCCGGCTCCACTTATAGCAACTACTGTCTGGGCTGG |
| H22- | | TTTCGCCAGACAACCGGAAAAGAAAGAGAAGGCGTGGCCGTGATTAACTGGGTC |
| DR591 | | GGCGGGATGCTCTACTTTGCCGATAGTGTGAAAGGTCGTTTCACGGTCTCCCAG |
| | | GACCAGGCCAAAAACACCGTCTACCTCCAGATGAACTCCCTCAAGCCTGAGGAT |
| | | ACTGCGATGTATTACTGCGCCGCTGAGTCCGTCTCTTCCTTCTCGTGCGGGGGC |
| | | TGGCTGACACGCCCCGACAGAGTCCCTTATTGGGGCCAAGGAACTCAGGTAACA |
| | | GTGTCTTCCGGTGGGGGTTCCCAGGTACAGCTTCAGGAGTCTGGTGGGGGTTCT |
| | | GTTCAGGCCGGGGGCTCCCTGCGCCTGTCCTGCACTGCTTCTGGCGCTATCGCA |
| | | AGCGGATATATTGATAGTCGCTGGTGTATGGCATGGTTCAGACAAGCCCCTGGG |
| | | AAGGAGCGCGAAGGCGTGGCGGCCATCTGGCCAGGCGGTGGGCTGACTGTCTAC |
| | | GCTGACAGCGTTAAAGGTCGCTTCACCATCTCAAGAGATCATGCTAAGAACACT |
| | | CTCTACCTCCAGATGAACAATCTGAAGCCTGAGGACACCGCCATGTATTACTGT |
| | | GCTGCGGGAAGCCCCCGCATGTGTCCTAGCCTGGAGTTCGGGTTCGACTACTGG |
| | | GGTCAGGGGACCCAGGTCACAGTGTCCAGC |
| hIL27 | 1496 | CAAGTGCAACTCCAAGAATCTGGAGGTGGCTCTGTGCAGGCGGGGGGTTCCCTG |
| Ra_VH | | AGACTGTCTTGCAGAGCAAGCGGCAGTACTTACTCTAACTACTGTTTGGGATGG |
| H22- | | TTCCGGCAGACAACCGGCAAAGAGAGGGAAGGCGTGGCCGTAATCAACTGGGTG |
| DR591 | | GGTGGCATGTTGTACTTCGCGGATAGCGTTAAGGGCCGCTTCACAGTTTCCCAG |
| | | GATCAGGCCAAGAACACCGTGTACTTGCAAATGAACTCTCTGAAGCCGGAGGAC |
| | | ACCGCCATGTATTACTGCGCTGCCGAGTCAGTATCTTCATTTAGCTGTGGCGGA |
| | | TGGTTGACTAGGCCGGACAGGGTCCCTTACTGGGGCCAGGGCACACAGGTGACT |
| | | GTGTCCTCTGGCGGAAGCGGGGGTTCAGGAGGCTCTGGGCAGGTGCAGTTGCAG |
| | | GAGAGCGGGGGGGGCTCTGTCCAGGCTGGAGGGAGCCTCAGGCTCTCATGCACA |
| | | GCCTCTGGGGCTATCGCATCAGGATATATTGATTCTCGGTGGTGCATGGCTTGG |
| | | TTCAGACAGGCACCGGGAAAGGAGCGCGAAGGTGTGGGGGCCATCTGGCCTGGC |
| | | GGGGGCCTGACCGTGTATGCCGATTCCGTGAAGGGTCGTTTCACCATCTCCCGT |
| | | GACCACGCGAAGAATACCCTGTATTTGCAGATGAATAACCTGAAGCCCGAAGAT |
| | | ACCGCCATGTATTACTGCGCTGCCGGGAGCCCACGTATGTGCCCTTCTTTGGAG |
| | | TTCGGTTTCGATTACTGGGGTCAGGGGACCCAGGTGACCGTCTCCTCC |
| hIL27 | 1497 | CAGGTGCAACTCCAGGAGAGCGGCGGGGGCAGTGTGCAGGCCGGAGGTTCCTTG |
| Ra_VH | | CGCCTGTCTTGTCGTGCGTCAGGAAGCACATACAGCAACTACTGGCTCGCGGGG |
| H22- | | TTCAGGCAGACTACCGGCAAGGAACGTGAAGGTGTAGCCGTGATTAACTGGGTG |
| DR592 | | GGCGGAATGCTGTACTTCGCGGACTCCGTGAAGGGCCGCTTCACCGTGTCTCAG |
| | | GACCAGGCCAAGAACACCGTTTATCTCCAGATGAACTCCCTGAAGCCGGAGGAC |
| | | ACGGCCATGTATTACTGCGCTGCCGAGTCCGTGTCTTCATTCCCCTGCGGTGGC |
| | | TGGTTGACTCGCCCTGATCGCGTGCCCTATTGGGGTCAGGGCACACAGGTGACA |
| | | GTCAGCTCTGGTGGAGGTTCACAGGTCCAGCTCCAGGAGAGTGGGGGGGGTAGC |
| | | GTTCAGGCCGGAGGCTCCCTGCGCCTGAGCTGTACCGCCCCAGGCTTTACCTCT |
| | | AATAGCTGTGGAATGGACTGGTATCGTCAGGCTCCTGGTAAGGAACGCGAGTTC |
| | | GTCTCTAGCATCTCAACCGACGGGACTACCGGCTATGCGGACTCTGTGAAGGGT |
| | | CGTTTCACCATTAGCAAGGACAAGGCCAAGGACACAGTGTACCTTCAGATGAAC |
| | | TCACTGAAGCCCGAGGACACTGGCATGTATAGCTGCAAAACTAAAGATGGAACC |
| | | ATCGCCACTATGGAACTGTGCGATTTCGGCTACTGGGGCCAGGGCACGCAGGTG |
| | | ACCGTGTCTTCC |
| hIL27 | 1498 | CAGGTGCAGTTGCAGGAGAGTGGTGGAGGCAGTGTGCAGGCCGGTGGCTCTCTT |
| Ra_VH | | AGACTGAGCTGCCGCGCCAGTGGCTCCACCTACTCCAACTATTGTCTGGGATGG |
| H22- | | TTTCGTCAGACCACTGGTAAAGAGCGGGAGGGGGTGGCCGTCATCAACTGGGTT |
| DR592 | | GGGGGTATGCTGTACTTTGCTGACTCTGTCAAGGGGCGGTTCACAGTCTCACAG |
| | | GACCAAGCCAAGAATACCGTGTACCTCCAGATGAACAGTCTGAAGCCGGAGGAT |
| | | ACCGCTATGTATTACTGCGCAGCCGAATCCGTCAGCTCTTTCTCCTGCGGAGGC |
| | | TGGCTCACGCGCCCTGACAGGGTTCCCTACTGGGGCCAAGGAACGCAGGTGACA |
| | | GTTTCTAGTGGTGGGTCCGGGGGTAGCGGAGGGTCCGGCCAGGTGCAGCTCCAG |
| | | GAATCCGGGGGAGGTTCCGTGCAGGCCGGAGGCTCCCTCCGCCTGAGCTGCACA |
| | | GCCCCCGGTTTCACCTCCAATTCTTGGGGCATGGACTGGTATCGCCAGGGTCCG |
| | | GGCAAGGAGCGTGAGTTCGTGTCCAGTATTTCTACGGACGGCACTACCGGGTAC |
| | | GCTGACTCCGTTAAGGGCCGCTTTACCATTAGCAAAGACAAGGCGAAGGACACA |
| | | GTCTATCTCCAGATGAATAGTGTGAAGCCGGAGGATACCGGCATGTACTCATGC |
| | | AAAACAAAGGATGGCACGATTGCGACAATGGAGCTGTGCGACTTCGGGTATTGG |
| | | GGCCAGGGCACGCAGGTGACGGTGTCCTCC |
| hIL27 | 1499 | CAGGTACAGCTCCAGGAATCCGGTGGGGGTTCAGTTCAGGCCGGGGGTAGCCTT |
| Ra_VH | | CGGCTGTCCTGCCGCGCGAGTGGCTCCACCTATAGCAACTACTGCCTGGGATGG |
| H22- | | TTCCGCCAGACCACAGGCAAGGAGCGTGAGGGCGTCGCTGTAATAAATTGGGTT |
| DR593 | | GGGGGAATGTTGTACTTTGCGGACAGTGTGAAGGGACGTTTCACCGTCAGTCAG |
| | | GACCAGGCCAAGAACACCGTTTACCTCCAGATGAACAGCCTCAAGCCCGAGGAT |
| | | ACCGCCATGTATTACTGCGCGGCTGAGTCTGTTTCTTCCTTCTCTTGTGGGGGC |
| | | TGGCTGACCAGGCCCGACAGAGTCCCATACTGGGGCCAGGGGACCCAGGTGACA |
| | | GTTAGCTCCGGGGGGCTCACAGGTGCAGCTGCAAGACGAGCCGGCGGTGGCTCC |
| | | GTCCAAGCGGGGGGGTCACTCAGACTGTCTTGCGCAGCTAGTGGTTACCCGTAT |
| | | TCAAACGGTTACATGGGATGGTTTAGACAGGCACCAGGCAAAGAGCGCGAAGGC |
| | | GTCGCTACCATCTACACAGGAGATGGCCGCACCTATTACGCCGACAGCGTGAAA |
| | | GGCAGGTTTACAATTAGTCGCGACAACGCCAAGAACACTGTCGATCTTCAGATG |
| | | TCTTCCTTGAAGCCAGAAGACACCGCCATGTATTACTGTGCAGCGCGGGCAGCC |
| | | CCACTTTATTCCAGCGGCTCTCCCCTCACCCGCGCCCGGTATAACGTCTGGGGC |
| | | CAAGGTACTCAGGTGACCGTGAGTTCT |
| hIL27 | 1500 | CAGGTGCAGTTGCAGGAATCAGGTGGAGGCTCTGTGCAGGCCGGTGGATCTCTG |
| Ra_VH | | CGGCTCAGCTGCCGGGCGTCCGGGAGCACTTACAGCAATTACTGTTTGGCGGGG |
| H22- | | TTTCGGCAGACCACTGGTAAGGAGAGAGAGGGAGTGGCCGTTATCAACTGGGTC |
| DR593 | | GGAGGTATGGTCTACTTCGCTGACAGCGTCAAGGGACGCTTCACCGTGTCCCAG |
| | | GACCAGGCTAAGAATACCGTTTATCCCCAGATGAATAGCCTGAAGCCTGAGGAC |
| | | ACCGCTATGTATTACTGTGCGGCTGAGAGCGTCAGCTCTTTCTCCTGCGGTGGA |
| | | TGGCTCACACGCCCCGACCGTGTCCCATATTGGGGACAAGGCACTCAGGTGACC |
| | | GTCAGCTCAGGAGGCAGCGGGGGTAGCGGGGGTTCCGGCCAAGTGCAGCTCCAG |
| | | GAGTCTGGGGGCGGAAGCGTGCAGGCCGGGGGAGCTTGCGCCTGTCCCTGTGCC |
| | | GCGTCTGGCTACCCTTACAGTAACGGCTATATGGGCTGGTTCCGGCAGGCCCCT |
| | | GGCAAGGAAAGGGAGGGTGTGGCCACTATCTACACTGGTGATGGTAGGACATAT |
| | | TACGCCGATAGCGTGAAGGGCCGGTTCACGATCTCTCGTGATAACGCCAAAAAT |
| | | ACCGTGGATCTTCAGATGTCATCCCTCAAGCCCGAGGATACCGCTATGTACTAT |
| | | TGCGCAGCCCGCGCTGCCCCGCTCTACAGCAGTGGAAGCCCGTTGACCCGCGCC |
| | | CGTTACAACGTGTGGGGTCAGGGCACACAAGTCACTGTCTCCTCA |
| hIL27 | 1501 | CAAGTCCAACTTCAGGAGTCAGGGGGAGGTTCTGTTCAAGCCGGGGGTTCACTG |
| Ra_VH | | AGGCTCTCTTGCCGCGCCTCCGGCTCAACATACTCCAACTATTGTCTGGGCTGG |
| H22- | | TTCAGGCAGACAACCGGGAAGGAGCGCGAGGGTGTGGCCGTCATCAACTGGGTC |
| DR594 | | GGGGGGATGCTGTACTTTGCAGATTCCGTGAAGGGCCGGTTTACAGTGAGCCAG |
| | | GACCAGGCTAAGAACACCGTGTACCTGCAAATGAACTCTCTGAAACCCGAGGAT |
| | | ACAGCGATGTATTACTGCGCTGCGGAGTCCGTGTCTAGCTTCTCTTGCGGGGGG |
| | | TGGCTGACCCGTCCTGATAGAGTCCCATACTGGGGACAGGGGACACAGGTGACC |
| | | GTGAGCAGTGGGGGTGGCAGCCAGGTGCAGCTTCAGGAAAGCGGAGGTGGCTCA |
| | | GTACAGGCGGGAGGCAGTCTGCGCCTGTCTTGCGTGGCCTCCGCATCCACCTAT |
| | | TGCACCTACGACATGCACTGGTACAGGCAGGCACCTGGCAAGGGTCGCGAGTTT |
| | | GTCAGTGCTATCGACTCCGACGGCACCACTCGGTACGCAGATTCCGTCAAGGGC |
| | | CGGTTTACCATCTCCCAGGGCACAGCTAAAAACACTGTTTACCTCCAAATGAAC |
| | | TCCCTCCAGCCTGAGGATACCGCTATGTATTACTGCAAGACAGTCTGTGTAGTG |
| | | GGCTCCCGTTGGTCCGATTACTGGGGCCAGGGGACCCAAGTTACGGTTTCTAGC |
| hIL27 | 1502 | CAAGTCCAACTCCAGGAAAGCGGGGGTGGCTCAGTCCAAGCAGGGGGCTCTCTC |
| Ra_VH | | AGACTGAGTTGCAGGGCAAGCGGTTCCACCTACTCAAACTACTGCCTCGGTTGG |
| H22- | | TTTCGGCAGACTACCGGCAAGGAAAGGGAGGGCGTGGCGGTCATTAACTGGGTA |
| DR594 | | GGGGGTATGGTCTACTTTGCAGACTCCGTGAAGGGCAGATTTACAGTATCCCAG |
| | | GACCAGGCTAAGAACACGGTGTACCTGCAAATGAACTCTCTGAAGCCAGAGGAC |
| | | ACCGCCATGTACTATTGCGCGGCAGAGTCAGTGAGCAGTTTTTCTTGTGGGGGC |
| | | TGGCTCACCCGCCCGGACCGCGTGCCTTACTGGGGACAAGGAACGCAGGTTACA |
| | | GTCTCTTCCGGCGGAAGCGGGGGCAGTGGTGGCAGCGGTCAGGTTCAGCTTCAG |
| | | GAGAGTGGGGGAGGCAGCGTGCAGGCAGGGGGTTCCCTGAGGCTGTCCTGTGTG |
| | | GCCTCTGCGAGTACGTATTGCACCTACGACATGCACTGGTATCGCCAGGGTCCT |
| | | GGTAAGGGCCGTGAGTTCGTGTCCGCCATCGACAGCGACGGAACCACACGGTAC |
| | | GCTGATTCCGTGAAAGGCAGGTTCACTATTTCCCAAGGAACTGCCAAAAACACT |
| | | GTCTACCTCCAGATGAACAGCCTCCAGCCTGAGGATACAGCCATGTACTATTGG |
| | | AAGACTGTGTGCGTGGTCGGCTCTCGTTGGTCCGACTACTGGGGCCAGGGCACC |
| | | CAGGTCACCGTGTCCAGT |
| hIL27 | 1503 | CAGGTGCAGGTCCAGGAGAGCGGTGGCGGGAGTGTTCAGGGGGGGGGTAGCCTG |
| Ra_VH | | AGACTGTCATGCAGAGCATCTGGCTCTACGTACTCTAACTACTGTCTTGGGTGG |
| 22- | | TTCCGCCAGACGACCGGAAAGGAGCGTGAGGGCGTCGCTGTGATTAACTGGGTG |
| DR595 | | GATCAGGCTAAGAACACTGTGTACCTTCAGATGAACAGGTTGAAGCCCGAAGAC |
| | | ACTGCCATGTACTATTGTGCAGCTGAATCTGTGAGCAGTTTCTCATGGGGTGGG |
| | | TGGCTTACCAGACCGGATAGAGTGCCTTACTGGGGCCAGGGGACCCAGGTTACC |
| | | GTGTCTAGCGGGGGTGGATCTCAGGTGCAGCTCCAGGAGTCCGGGGGGGGCTCC |
| | | GTTCAGGCTGGGGGTAGTCTCACTCTGTCCTGTGCCGCTTCTGAGTACGCCTAC |
| | | AGTACATGCAACATGGGCTGGTATCGCCAGGCTCCGGGGAAGGAACGCGAACTT |
| | | GTGAGCGCCTTCATCTCCGATGGCTCTACCTATTACGCCGACTCCGTGAAGGGG |
| | | AGGTTTACCATCACTCGCGACAACGCTAAGAACACTGTCTACTTGCAGATGAAC |
| | | TCACTGAAACCCGAGGATACTGCCATCTATTACTGTTCCGCTAACTGCTATAGG |
| | | CGGCTGAGAAACTATTGGGGACAGGGGACCCAGGTGACTGTCTCTAGC |
| hIL27 | 1504 | CAAGTGCAACTCCAGGAAAGTGGAGGTGGCTCCGTGCAAGCCGGAGGCAGTCTC |
| Ra_VH | | CGCCTGAGCTGTCGTGCGAGCGGTTCCACCTATAGCAACTACTGCCTGGGATGG |
| H22- | | TTTAGACAGACAACCGGGAAGGAGCGGGAGGGAGTCGCGGTGATAAATTGGGTG |
| DR595 | | GGCGGTATGCTGTACTTCGCGGATTCTGTCAAGGGGAGATTCACCGTGAGCCAG |
| | | GATCAGGCGAAGAACACTGTGTACCTTCAGATGAACTCTCTCAAGCCGGAGGAC |
| | | ACAGCCATGTACTATTGTGCTGCCGAGTCTGTTTCATCCTTCTCGTGTGGCGGT |
| | | TGGCTGACCCGCCCAGATCGCGTGCCTTACTGGGGCCAGGGTACTCAGGTCACC |
| | | GTCTCTTCCGGGGGTTCAGGCGGTAGCGGTGGCTCCGGCCAGGTTCAACTCCAG |
| | | GAAAGCGGAGGCGGTTCTGTGCAAGCCGGGGGCTCCCTGACCCTGTCCTGTGCT |
| | | GCCAGCGAGTACGCCTATAGCACTTGTAACATGGGATGGTATCGCCAGGCCCCA |
| | | GGAAAAGAACGCGAGCTTGTGAGTGCTTTTATCTCTGACGGGTCCACCTATTAC |
| | | GCGGACTCTGTGAAAGGTCGCTTCACAATCACCCGCGATAACGCAAAGAACACT |
| | | GTCTACCTTCAGATGAACTCCCTGAAGCCCGAGGACACTGCGATTTATTACTGT |
| | | AGCGCCAACTGTTACCGCCGTCTCCGCAACTACTGGGGACAGGGGACCCAGGTG |
| | | ACCGTCTCATCC |
| hIL27 | 1505 | CAGGTCCAACTTCAGGAGTCTGGCGGAGGCAGCGTGCAAGCGGGTGGGAGCCTG |
| Ra_VH | | CGCCTTTCCTGCCGCGCGTCTGGCAGCACCTACTCCAACTACTGCCTGGGATGG |
| H22- | | TTTCGCCAGACCACAGGTAAGGAGCGTGAAGGTGTGGCCGTCATCAACTGGGTC |
| DR596 | | GGCGGTATGGTGTATTTCGCTGATTCCGTTAAGGGCAGATTTACTGTGAGTCAG |
| | | GACCAGGCAAAGAACACAGTGTACCTTCAGATGAATAGCCTGAAGCCCGAAGAC |
| | | ACTGCCATGTACTATTGCGCAGCCGAATCTGTGTCTAGCTTTTCCTGCGGTGGG |
| | | TGGCTGACCCGCCCTGATCGCGTGCCATACTGGGGGCAGGGCACCCAGGTGACT |
| | | GTATCTTCCGGGGGAGGCTCCCAGGTCCAGTTGCAGGAGTCCGGCGGAGGCCTG |
| | | GTACAGCCTGGTGGATCTCTCCGCCTCTCTTGCACCGCGTCCGGGCTTACCTTC |
| | | GATGACTCTGTCATGGGCTGGTTTCGGCAGGCTCCTGGTAAGGGCCGTGAAGCC |
| | | GTGTCCTGCATCTCCTCTTCAGGAGCGAACGCTTTCTATGCTGATAGCGTTAAA |
| | | GGCCGTTTCACCATCTCCCGTGATAACGCTAAGAACACCCTCTACCTTCAAATG |
| | | AACTCCCTCAAGCCAGAGGACACCGCCACATATTACTGTAAACGGGGCCATGCT |
| | | TGCGCGGGATATTACCCCATCCCTTATGACGATTACTGGGGTCAAGGGACACAG |
| | | GTCACTGTCAGCTCC |
| hIL27 | 1506 | CAGGTGCAACTTCAGGAGTCCGGTGGGGGTTCCGTGCAGGCTGGTGGCAGCCTG |
| Ra_VH | | AGGCTGAGCTGTCGCGCCAGGGGGTCCACCTACTCTAACTATTGTCTGGGATGG |
| H22- | | TTCCGGCAGACCACAGGCAAGGAGCGCGAGGGAGTCGCTGTAATCAACTGGGTA |
| DR596 | | GGTGGGATGGTGTACTTCGCCGATAGTGTAAAAGGCCGGTTCACCGTGTCACAG |
| | | GACCAAGCCAAGAACACAGTCTACCTCCAGATGAACTCCCTGAAGCCAGAGGAC |
| | | ACCGCCATGTATTACTGCGCCGCTGAATCCGTCAGCTCTTTCTCTTGGGGCGGT |
| | | TGGCTGACCCGCCCCGACCGTGTCCCCTACTGGGGCCAGGGCACTCAGGTGACT |
| | | GTGAGCAGTGGTGGCAGCGGGGGCTCCGGTGGAAGCGGCCAGGTTCAGCTCCAG |
| | | GAATCCGGGGGCGGACTCGTCCAGCCAGGGGGCAGCCTGCGGCTGTCCTGCACC |
| | | GCAAGCGGCCTGACCTTCGACGATAGCGTCATGGGATGGTTCCGTCAAGCCCCT |
| | | GGCAAGGGGAGGGAGGCCGTCAGCTGTATTAGCTCTAGCGGCGCTAACGCCTTC |
| | | TATGCCGATTCTGTTAAGGGTAGGTTCACAATCTCCCGCGACAACGCCAAAAAT |
| | | ACGCTGTATCTCCAGATGAACAGCCTGAAGCCCGAGGACACCGCAACCTATTAC |
| | | TGTAAGAGGGGACACGCATGTGCCGGTTATTACCCTATCCCCTACGATGACTAC |
| | | TGGGGGCAAGGTACTCAGGTCACCGTTTCCTCA |
| hIL27 | 1507 | CAGGTGCAACTTCAGGAGAGTGGAGGCGGTAGCGTCCAAGCTGGGGGTTCCCTG |
| Ra_VH | | AGACTTTCATGTAGAGCTTCCCGTTCTCCTTACGGAAATTACTGTCTGGGCTGG |
| H23- | | TTCCGCCAGTCCACTGGTAAGGAACGCGAGGGTGTGGCTGTGATAAATTGGGTG |
| DR591 | | GGTGGAATGCTGTACTTTGCCGACTCTGTTAAGGGTAGATTCACTGTGTCCCAA |
| | | GATCATGCCAAGAACACCGTCACCCTTCAGATGAACTCCCTGAAGCCGGAAGAC |
| | | ACCGCTATGTACTATTGTGCTGCCGAGTCCGTGTCCTCTTTCTCTTGCGGCGGT |
| | | TGGCTGACCAGACCGGACAGGGTTCCGTATTGGGGCCAAGGCACCCAGGTCACT |
| | | GTGAGTTCTGGAGGGGGTTCCCAGGTCCAACTCCAAGAAAGCGGAGGCGGTTCC |
| | | GTCCAGGCTGGCGGTTCTCTGCGCCTGAGCTGCACCGCCAGCGGGGCCATTGCG |
| | | TCTGGTTACATCGACAGTAGATGGTGTATGGCGTGGTTTCGGCAGGCTCCTGGA |
| | | AAGGAACGCGAGGGCGTTGCTGCCATCTGGCCAGGGGGTGGCCTGACAGTCTAT |
| | | GCGGACTCCGTGAAGGGGCGCTTCACCATTAGCAGAGACCACGCAAAAAACACT |
| | | CTGTATTTGCAGATGAATAACCTGAAGCCCGAAGACACTGCCATGTACTATTGT |
| | | GCTGCCGGTTCTCCGCGCATGTGCCCATCCCTGGAGTTCGGATTCGACTACTGG |
| | | GGACAGGGCACCCAGGTGACGGTGTCCAGT |
| hIL27 | 1508 | CAGGTCCAGTTGCAGGAGTCAGGGGGGGGTAGCGTCCAGGCAGGGGGCAGCCTG |
| Ra_VH | | CGCCTGTCCTGCCGGGCCAGCAGAAGCCCCTACGGAAATTACTGTCTGGGCTGG |
| H23- | | TTCCGGCAGAGCACTGGGAAGGAGCGCGAGGGCGTCGCCGTAATCAACTGGGTC |
| DR591 | | GGCGGGATGTGTATTTCGCCGACAGTGTGAAGGGACGGTTTACCTGTGAGTCAG |
| | | GACCACGCAAAGAATACAGTCACGTTGCAGATGAACTCCCTGAAGCCTGAGGAC |
| | | ACCGCTATGTACTATTGCGCGGCTGAGTCAGTGTCTAGCTTCAGGTGTGGTGGC |
| | | TGGCTCACTCGTCCTGATCGCGTACCATATTGGGGTCAGGGGACTCAGGTCACC |
| | | GTGAGTAGCGGCGGAAGTGGAGGCAGCGGTGGGAGCGGCCAAGTTCAGTTGCAG |
| | | GAGAGCGGCGGGGGCTCTGTGCAGGCTGGTGGCTCCCTGCGTCTGTCCTGCACC |
| | | GCGTCCGGGGCCATCGCCAGCGGATACATCGACAGTAGGTGGTGTATGGCATGG |
| | | TTCCGTCAGGCCCCTGGTAAGGAGCGCGAGGGCGTGGCCGCTATTTGGCCGGGT |
| | | GGGGGACTCACCGTGTATGCTGACTCCGTAAAGGGTCGCTTCACTATCTCTCGC |
| | | GATCACGCCAAGAACACCCTGTACTTGCAGATGAACAATCTGAAGCCCGAGGAC |
| | | ACCGCTATGTATTACTGCGCAGCTGGAAGCCCCAGGATGTGTCCGAGCCTGGAG |
| | | TTCGGCTTCGACTACTGGGGGCAGGGGACTCAGGTGACCGTGTCCTCT |
| hIL27 | 1509 | CAAGTCCAGCTTCAGGAAAGCGGGGGTGGAAGCGTGCAGGCTGGAGGCAGCCTG |
| Ra_VH | | AGACTCAGCTGCCGCGCTTCAAGGTCCCCCTACGGTAACTACTGCCTGGGCTGG |
| H23- | | TTTCGCCAGAGTACAGGCAAGGAAAGGGAAGGTGTGGCCGTTATCAACTGGGTG |
| DR592 | | GGCGGTATGCTGTACTTTGCCGACTCCGTCAAGGGCCGTTTTACAGTGTCCCAG |
| | | GACCACGCCAAGAACACGGTCACGCTTCAGATGAACAGCCTGAAGCCCGAGGAC |
| | | ACCGCTATGTATTACTGTGCTGCCGAGTCCGTTAGCAGTTTCTCCTGGGGCGGA |
| | | TGGCTGACCCGCCCTGACCGCGTTCCCTACTGGGGGCAGGGCACTCAGGTGACG |
| | | GTCAGCTCTGGGGGGGGGTCCCAGGTTCAGCTGCAAGAGAGTGGTGGAGGCTCC |
| | | GTACAGGCCGGGGGATCTCTTCGCCTGTCCTGTACTGCTCCGGGCTTCACGAGT |
| | | AACTCCTGTGGGATGGACTGGTATCGCCAAGCGCCCGGCAAGGAGCGCGAGTTC |
| | | GTAAGTAGCATCTCAACAGACGGCACGACCGGGTACGCCGATTCCGTGAAGGGC |
| | | CGCTTTACCATTAGCAAGGACAAGGCCAAGGACACTGTGTATCTGCAAATGAAC |
| | | TCCCTGAAACCCGAGGATACGGGCATGTACTCTTGCAAGACTAAGGATGGCACG |
| | | ATTGCCACAATGGAGGTGTGCGATTTCGGATATTGGGGCCAGGGCACCCAGGTG |
| | | ACTGTCTCCTCT |
| hIL27 | 1510 | CAGGTGCAACTTCAGGAAAGTGGGGGGGGTTCCGTGCAGGCAGGGGGCTCTCTG |
| Ra_VH | | CGTCTGTCTTGCAGAGCCAGCAGGTCTCCTTATGGTAACTACTGCCTCGGGTGG |
| H23- | | TTTCGTCAGTCTACCGGAAAGGAGCGCGAAGGAGTCGCAGTGATAAATTGGGTC |
| DR592 | | GGCGGTATGGTCTATTTCGCCGACAGCGTCAAGGGGCGTTTCACCGTCAGCCAA |
| | | GATCACGCTAAGAACACCGTAACACTGCAAATGAACTCCCTCAAACCTGAAGAC |
| | | ACGGCTATGTATTACTGCGCAGCGGAAAGTGTGAGCAGTTTCAGTTGTGGTGGA |
| | | TGGCTGACCCGCCCAGATCGCGTCCCTTACTGGGGGCAGGGCACCCAGGTGACT |
| | | GTCTCCAGTGGAGGGTCCGGTGGCAGCGGGGGAAGCGGCCAGGTGCAGTTGCAG |
| | | GAGTCTGGGGGGGGTTCTGTCCAGGCTGGAGGTAGCCTCCGCCTGTCTTGCACA |
| | | GCCCCCGGCTTTACGTCCAACTCTTGTGGCATGGACTGGTATCGGCAGGCTCCA |
| | | GGCAAAGAACGCGAGTTCGTCAGCTCTATCTCTACTGATGGAACTACAGGATAC |
| | | GCAGACAGCGTTAAGGGGCGTTTCACCATCCCCAAAGATAAAGCTAAGGACACC |
| | | GTTTACCTGCAAATGAACTCACTGAAGCCAGAGGATACAGGTATGTACTCTTGC |
| | | AAGACTAAGGATGGGACCATCGCCACTATGGAACTGTGTGATTTCGGCTACTGG |
| | | GGCCAGGGCACCCAGGTCACCGTGAGCAGC |
| hIL27 | 1511 | CAGGTGCAACTGCAAGAAAGTGGCGGTGGAAGCGTGCAGGCAGGTGGAAGCCTG |
| Ra_VH | | CGCCTCTCTTGTCGTGCTTCCAGAAGTCCGTATGGCAATTATTGTCTGGGCTGG |
| H23- | | TTTAGACAGTCCACTGGTAAGGAAAGGGAAGGAGTGGCTGTCATCAACTGGGTG |
| DR593 | | GGCGGTATGCTGTACTTCGCCGATTCCGTCAAGGGCCGTTTCACAGTCTCCCAA |
| | | GACCATGCAAAGAACACAGTGACACTCCAGATGAACTCCCTCAAGCCCGAGGAC |
| | | ACTGCTATGTATTACTGCGCCGCTGAGTCTGTCTCCAGCTTCTCTTGCGGCGGA |
| | | TGGCTGACTCGCCCGGATCGCGTTCCCTATTGGGGCCAGGGGACGCAGGTGACC |
| | | GTGTCCTCAGGCGGGGGCTCCCAGGTTCAGCTCCAGGAGTCTGGGGGGGGCTCA |
| | | GTACAGGCCGGTGGCTCCCTCCGCCTCTCATGCGCAGCCTCCGGCTACCCGTAT |
| | | AGCAACGGTTACATGGGCTGGTTCAGGCAGGCCCCTGGGAAGGAACGCGAGGGC |
| | | GTGGCGACTATCTACACTGGCGACGGTAGGACTTATTACGCTGATTCAGTGAAG |
| | | GGCAGGTTCACAATTTCTCGCGACAACGCTAAAAATACCGTGGATCTCCAGATG |
| | | TCCAGCCTGAAACCTGAGGATACTGCCATGTATTACTGTGCCGCTCGCGCAGCT |
| | | CCTCTGTATAGTAGCGGCAGCCCCTTGACCCGCGCTAGATACAATGTCTGGGGT |
| | | CAGGGCACCCAGGTAACCGTTAGTTCC |
| hIL27 | 1512 | CAGGTACAACTTCAGGAAAGCGGAGGGGGTCCGTGCAGGCCGGCTGGCTCACTG |
| Ra_VH | | CGCTTGAGTTGTCGCGCCAGCCGCTCCCCTTATGGGAACTACTGCCTTGGGTGG |
| H23- | | TTCCGCCAGAGCACTGGAAAGGAGCGCGAGGGCGTCGCAGTCATCAACTGGGTT |
| DR593 | | GGCGGGATGCTCTATTTCGCCGACAGCGTGAAAGGGCGGTTTACCGTTAGCCAA |
| | | GACCACGCCAAGAACACAGTGACCCTTCAAATGAACTCCCTGAAGCCCGAAGAC |
| | | ACCGCTATGTACTATTGCGCCGCAGAGTCAGTGTCCAGTTTTAGGTGGGGAGGC |
| | | TGGTTGACTCGCCCTGACCGCGTGCCTTATTGGGGCCAGGGCACCCAGGTGACA |
| | | GTAAGCAGTGGAGGCAGCGGCGGTTCCGGTGGAAGCGGCCAGGTGCAGCTTCAG |
| | | GAGAGCGGCGGTGGCAGTGTTCAGGCTGGCGGGTCCCTGAGGCTGTCCTGTGCT |
| | | GCCTCCGGCTACCCCTACTCCAACGGTTACATGGGTTGGTTCCGCCAAGCGCCA |
| | | GGAAAGGAACGCGAGGGTGTGGCTACCATCTACACCGGCGATGGTCGCACTTAT |
| | | TACGGTGACAGCGTGAAAGGCCGGTTCACAATCAGCCGGGACAACGCCAAGAAC |
| | | ACAGTGGACCTGCAAATGTCCAGTTTGAAGCCTGAAGACACAGCCATGTATTAC |
| | | TGTGCGGCTCGGGCCGCGCCCCTCTACAGCTCCGGCTCACCACTGACACGCGCG |
| | | AGATACAACGTGTGGGGACAGGGCACCCAGGTGACAGTTAGCAGG |
| hIL27 | 1513 | CAGGTTCAGTTGCAGGAGTCTGGTGGCGGTTCCGTCCAGGCTGGGGGAAGTCTG |
| Ra_VH | | CGCCTCAGTTGTCGTGCCAGCCGCTCTCCTTACGGCAACTATTGCCTGGGCTGG |
| H23- | | TTCCGGCAGTCCACTGGCAAGGAGAGGGAAGGAGTCGCCGTTATCAACCGGGTG |
| DR594 | | GGTGGAATGCTCTACTTTGCTGATAGCGTGAAGGGCCGGTTCACAGTTTCTCAG |
| | | GACCATGCGAAGAACACTGTTACTTTGCAGATGAACTCCCTGAAGCCAGAGGAT |
| | | ACGGCTATGTATTACTGTGCAGCCGAGTCCGTGAGTTCCTTTTCCTGCGGGGGT |
| | | TGGCTCACCCGTCCTGACCGTGTGCCTTATTGGGGCCAGGGTACGCAAGTCACC |
| | | GTAAGTAGCGGAGGGGGCAGCCAGGTCCAGTTGCAGGAGTCCGGCGGAGGCTCC |
| | | GTGCAGGCCGGGGGTTCTCTGCGTCTCTCATGTGTGGCATCTGCTTCTACTTAC |
| | | TGCACTTACGACATGCACTGGTATCGGCAGGCTCCTGGGAAAGGACGGGAGTTC |
| | | GTTAGTGCCATCGACAGCGACGGCACTACACGCTATGCTGATTCCGTTAAGGGC |
| | | CGCTTTACGATTTCACAGGGGACGGCCAAGAACACTGTGTACCTCCAGATGAAG |
| | | TCTTTGCAGCCTGAGGACACGGCTATGTATTACTGCAAAACCGTTTGTGTAGTG |
| | | GGCAGCCGCTGGTCAGACTATTGGGGCCAGGGCACACAGGTCACCGTGAGTTCT |
| hIL27 | 1514 | CAGGTGCAACTGCAAGAGTCCGGCGGAGGCTCCGTGCAGGCTGGTGGCTCACTC |
| Ra_VH | | CGGCTCTCCTGTCGCGCCTCTCGCAGCCCTTATGGGAATTACTGCCTGGGTTGG |
| H23- | | TTCCGTCAGAGTACCGGCAAGGAGCGTGAGGGAGTCGCAGTAATCAACTGGGTC |
| DR594 | | GGAGGTATGCTGTACTTCGCCGACTCCGTGAAGGGAAGGTTCACTGTGTCCCAG |
| | | GACCACGCCAAGAACACGGTAACGCTCCAGATGAACTCCCTGAAGCCCGAAGAG |
| | | ACCGCTATGTATTACTGTGCTGCCGAGTCCGTGTCCTCATTCTCGTGCGGCGGT |
| | | TGGCTGACACGCCCAGACCGCGTGCCCTATTGGGGACAGGGTACACAGGTTACC |
| | | GTCAGCTCTGGCGGGTCTGGTGGCAGTGGGGGCTCCGGTCAGGTGCAGTTGCAG |
| | | GAGTCCGGCGGAGGCTCTGTGCAGGCTGGTGGGAGTCTGCGCCTGTCTTGCGTC |
| | | GCCAGCGCCAGTACTTATTGTACTTACGACATGCACTGGTATAGACAGGCCCCC |
| | | GGTAAGGGAAGGGAGTTCGTGTCAGCTATCGACAGCGATGGGACCACTAGATAT |
| | | GCGGATAGCGTCAAGGGGCGCTTCACCATCTCCCAGGGAACCGCAAAAAACACT |
| | | GTCTACCTTCAGATGAACAGCCTCCAGCCTGAGGACACGGCTATGTATTACTGC |
| | | AAGACCGTGTGTGTCGTTGGATCTCGCTGGTCTGACTATTGGGGGCAGGGCACC |
| | | CAGGTGACGGTGTCCAGC |
| hIL27 | 1515 | CAGGTTCAGCTCCAGGAGTCTGGAGGGGGCAGCGTGCAGGCGGGAGGGTCTCTT |
| Ra_VH | | CGTCTTAGCTGCCGTGCCAGTCGCTCCCCCTACGGTAACTACTGCCTGGGGTGG |
| H23- | | TTTCGCCAGAGTACCGGCAAGGAGCGCGAAGGTGTGGCTGTAATCAACTGGGTT |
| DR595 | | GGGGGTATGGTGTATTTCGCCGACTCCGTGAAAGGTAGGTTTACAGTGAGCCAG |
| | | GACCACGCTAAGAACACCGTCACCCTCCAGATGAACTCTCTCAAACCGGAGGAT |
| | | ACCGCTATGTATTACTGCGCTGCCGAGAGTGTGAGTAGCTTCTCTTGTGGCGGT |
| | | TGGCTGACCAGGCCTGACCGCGTACCCTACTGGGGCCAGGGCACCCAGGTCACA |
| | | GTCAGCTCTGGTGGAGGCTCACAGGTGCAGCTTCAGGAGTCCGGGGGGGGCAGG |
| | | GTGCAGGCCGGGGGCTCACTGACCCTGAGCTGTGCTGCCAGCGAATACGCATAC |
| | | TCTACCTGCAATATGGGCTGGTATCGGCAGGCCCCTGGGAAAGAAAGGGAACTG |
| | | GTTTCTGCCTTTATCAGCGATGGATCAACATATTACGCCGACTCCGTGAAAGGG |
| | | AGGTTCACGATCACCCGCGACAACGCCAAGAACACTGTGTACCTCCAAATGAAC |
| | | AGCCTGAAGCCTGAGGACACCGCGATCTATTACTGCTCTGCTAACTGCTACCGC |
| | | AGGCTGCGCAATTATTGGGGTCAAGGCACCCAGGTTACCGTGTCTTCC |
| hIL27 | 1516 | CAGGTCCAGCTCCAGGAGTCTGGCGGAGGCTCCGTGCAGGCTGGAGGCAGCCTC |
| Ra_VH | | AGACTTTCTTGCAGAGCTTCCCGGTCTCCCTATGGAAACTACTGCTTGGGTTGG |
| H23- | | TTCCGCCAATCCACTGGCAAGGAGAGGGAGGGTGTTGCTGTCATCAACTGGGTG |
| DR595 | | GGCGGGATGGTTTATTTCGCCGACAGTGTCAAGGGACGGTTCACCGTCTCTCAG |
| | | GATCACGCCAAGAACACTGTGACCTTGCAGATGAACTCACTCAAACCCGAAGAT |
| | | ACCGCTATGTACTATTGTGCCGCAGAAAGCGTGTCTAGGTTCAGCTGTGGTGGC |
| | | TGGCTCACACGCCCTGACCGGGTGCCATACTGGGGCCAGGGCACGCAGGTGACC |
| | | GTAAGCTCAGGCGGGAGTGGTGGCTCTGGAGGCTCTGGACAGGTGCAGCTCCAG |
| | | GAGAGCGGGGGGGGGAGCGTGCAGGCTGGCGGTAGTCTGACCCTGTCATGCGCT |
| | | GCAAGCGAATACGCCTACAGCACCTGCAATATGGGGTGGTATCGTCAGGCTCCT |
| | | GGCAAGGAGCGTGAGCTGGTTTCCGCCTTCATGTCCGATGGCTCTACCTATTAC |
| | | GCCGACTCAGTGAAGGGCCGCTTCACTATCACTAGGGACAATGCCAAGAACACA |
| | | GTATACCTCCAGATGAACTCTCTGAAGCCAGAGGATACTGCCATCTATTACTGT |
| | | AGCGCCAACTGTTATCGTCGCCTGCGCAACTATTGGGGTCAAGGCACACAGGTG |
| | | ACAGTCTCTAGC |
| hIL27 | 1517 | CAGGTGCAACTCCAGGAATCCGGGGGTGGATCAGTACAGGCAGGCGGAAGCCTG |
| Ra_VH | | AGGCTGTCTTGTCGCGCTTCCCGTTCTCCCTACGGAAATTACTGTCTGGGCTGG |
| H23- | | TTCCGGCAGTCCACTGGAAAAGAGAGGGAGGGAGTGGCCGTTATCAACTGGGTA |
| DR596 | | GGAGGTATGCTGTACTTTGCCGACAGCGTGAAAGGAAGGTTCACCGTGAGCCAG |
| | | GACCACGCCAAGAATACCGTGACCTTGCAGATGAACAGCCTGAAGCCCGAAGAC |
| | | ACTGCCATGTACTATTGTGCCGCTGAGAGTGTATCATCTTTCTCCTGGGGTGGC |
| | | TGGTTGACTCGCCCAGACAGAGTGCCTTATTGGGGGCAGGGCACTCAGGTGACC |
| | | GTGTCTTCCGGTGGAGGCTCCCAGGTTCAACTTCAGGAGTCCGGTGGAGGCCTG |
| | | GTACAGCCTGGCGGATCTCTCAGGCTCAGCTGCACTGCCTCTGGACTGACCTTC |
| | | GATGACTCTGTTATGGGCTGGTTTAGGCAGGCCCCTGGAAAAGGCCGGGAGGCG |
| | | GTCAGCTGCATCAGCTCTAGCGGGGCCAATGCGTTTTACGCTGATTCCGTGAAG |
| | | GGCCGCTTCACTATCTCTAGGGACAACGCGAAGAACACCCTCTACTTGCAGATG |
| | | AACTCCCTCAAGCCCGAGGACACGGCGACTTATTACTGCAAAAGAGGACACGCT |
| | | TGTGCCGGATATTACCCGATTCCCTATGATGACTATTGGGGCCAGGGCACCCAG |
| | | GTGACAGTGAGCAGC |
| hIL27 | 1518 | CAGGTGCAACTCCAGGAAAGCGGCGGTGGAAGCGTGCAGGCTGGAGGGTCCCTC |
| Ra_VH | | AGACTGAGTTGCCGGGCCTCACGGTCCCCCTATGGGAACTATTGCCTGGGTTGG |
| H23- | | TTCCGTCAGTCCACTGGCAAGGAGCGCGAGGGCGTGGCCGTTATCAACTGGGTG |
| DR596 | | GGAGGGATGCTGTACTTTGCAGACAGCGTTAAGGGTCGGTTTACCGTGAGCCAG |
| | | GACCACGCCAAGAATACCGTGACCTTGCAGATGAACTCCCTGAAGCCTGAGGAC |
| | | ACCGCCATGTATTACTGCGCTGCCGAATCCGTGTCTAGCTTTAGCTGTGGGGGG |
| | | TGGCTCACCAGACCTGATCGCGTGCCGTACTGGGGCCAGGGCACTCAAGTGACG |
| | | GTCAGCTCCGGTGGAAGCGGTGGCTCCGGCGGTTCCGGGCAGGTGCAGTTGCAG |
| | | GAGTCAGGGGGAGGGTTGGTTCAGCCCGGTGGCTCCTTGCGTCTGTCCTGTACT |
| | | GCAAGCGGGCTGACCTTCGACGATTCTGTGATGGGCTGGTTTCGTCAGGCTCCT |
| | | GGCAAGGGCAGAGAGGCGGTGAGCTGTATCTCCAGCTCCGGCGCTAATGCCTTC |
| | | TACGCAGACAGTGTTAAGGGTAGGTTTACTATCTCCAGGGACAATGCTAAAAAC |
| | | ACTCTGTATGTTCAGATGAACAGCCTGAAGCCGGAGGATACTGCTACCTATTAC |
| | | TGTAAGCGGGGCCATGCTTGCGCCGGGTACTATCCAATCCCCTACGATGACTAT |
| | | TGGGGTCAAGGCACACAGGTGACAGTGTCCTCC |
| hIL27 | 1519 | CAGGTCCAGTTGCAGGAGAGCGGTGGGGGCCTGGTGCAGCCTGGCGGTTCCCTG |
| Ra_VH | | CGCCTGTGTTGTGCTGCGAGCGGGTTCACTTTCAGCCATTCTGGTATGAGTTGG |
| H24- | | GTACGCCAGGCTCCAGGCAAGGGCCTGGAATGGGTGAGCACCATCAACTCTGGA |
| DR591 | | GGTGCCTTCTACTTATTACACCGACAGTGTCAAGGGACGCTTACAATCTCCAGA |
| | | GACAACGCGAAGAATATGCTTTACTTGCAGCTCAACTCACTGAAGACCGAGGAT |
| | | ACTGCCATGTATTACTGCGCTAAGGGAGGCTCAGGATATGGTGATGCCTCTCGC |
| | | ATGACCTCTCCTGGCTCACAGGGAACCCAGGTGACCGTCAGTTCCGGTGGAGGT |
| | | TCTCAGGTCCAGCTCCAGGAGTCCGGGGGGGCAGCGTGCAGGCCCGGAGGCAGC |
| | | CTGCGCCTGTCATGCACAGCCTCTGGCGCTATCGCCAGTGGTTACATCGACAGC |
| | | AGGTGGTGCATGGCCTGGTTCAGACAGGCCCCTGGTAAGGAACGCGAGGGAGTT |
| | | GCTGCCATTTGGCCAGGGGGTGGCCTGACAGTGTACGCCGATAGCGTCAAGGGG |
| | | CGCTTCACTATCTCACGGGATCACGCTAAAAACACTTTGTACCTCCAGATGAAT |
| | | AACCTCAAACCAGAGGACACCGCGATGTATTACTGCGCTGCGGGCTCTCCCCGC |
| | | ATGTGTCCCTCTCTGGAGTTCGGGTTCGATTATTGGGGTCAGGGAACGCAGGTG |
| | | ACTGTGTCCTCC |
| hIL27 | 1520 | CAGGTGCAGCTTCAGGAGTCCGGGGGAGGCCTGGTTCAGCCCGGCGGTTCTCTC |
| Ra_VH | | CGCCTCAGCTGTGCCGCTTCCGGGTTTACATTCAGCCACAGTGGAATGTCTTGG |
| H24- | | GTACGTCAAGCGCCAGGTAAGGGCCTGGAGTGGGTCTCTACAATCAACAGCGGT |
| DR591 | | GGCGCAAGTACATACTATACCGACTCCGTGAAAGGGGGGTTTACCATCTCCCGT |
| | | GACAACGCTAAGAATATGCTCTATCTTCAGCTGAACTCACTCAAGACTGAGGAT |
| | | ACCGCTATGTATTACTGTGCGAAAGGGGGATCTGGGTACGGCGATGCGAGCCGC |
| | | ATGACCTCCCCCGGCAGTCAGGGCACTCAAGTGACCGTATCATCCGGGGGTTCC |
| | | GGTGGCTCAGGAGGTAGCGGCCAGGTTCAACTGCAAGAGAGTGGGGGGGGCTCC |
| | | GTGCAGGGGGGGGTTCCCTCAGGCTCTCTTGCACTGCAAGGGGCCGCTATTGCG |
| | | AGTGGGTACATCGACTCCAGATGGTGTATGGCGTGGTTCCGCCAGGCACCTGGC |
| | | AAGGAGCGCGAAGGCGTGGCAGCCATCTGGCCTGGGGGGGGTCTTACCGTCTAC |
| | | GCTGACTCCGTGAAAGGCCGCTTCACTATTTCTCGCGACCATGCCAAGAACACC |
| | | CTGTACTTGCAAATGAATAACCTGAAGCCTGAAGACACCGCGATGTATTACTGC |
| | | GCCGCTGGATGTCCCAGGATGTGCCCGAGCCTGGAGTTCGGTTTCGACTACTGG |
| | | GGCCAAGGCACACAGGTTACAGTCTCCTCC |
| hIL27 | 1521 | CAAGTGCAGCTCCAGGAGAGCGGTGGCGGTCTCGTGCAACCAGGAGGTTCCTTG |
| Ra_VH | | CGGCTGAGCTGTGCCGCGTCAGGTTTTACATTTAGCCACAGTGGGATGTCTTGG |
| H24- | | GTGAGACAGGCTCCGGGTAAGGGTCTCGAATGGGTCAGTACTATCAATTCCGGC |
| DR592 | | GGAGCAAGCACATACTATACCGACTCTGTCAAGGGGAGATTTACCATCCCCAGA |
| | | GACAATGCCAAAAACATGCTGTATTTGCAACTCAACAGTCTGAAGACCGAAGAC |
| | | ACCGCGATGTATTACTGCGCCAAGGGCGGTTCCGGGTACGGAGACGCGAGCCGC |
| | | ATGACTTCCCCTGGATCTCAGGGCACCCAGGTGACTGTCTCCAGCGGTGGCGGT |
| | | TCCCAAGTCCAACTCCAGGAAAGCGGAGGGGGTTCTGTGCAGGCAGGGGGGTCC |
| | | CTTCGGTTGAGCTGTACCGCACCCGGCTTTACCAGCAACTCTTGTGGTATGGAC |
| | | TGGTATCGGCAGGCTCCTGGCAAAGAGCGCGAGTTCGTGTCTTCCATTTCCACC |
| | | GACGGCACTACCGGGTACGCGGATTCCGTGAAGGGCAGGTTCACGATCAGCAAG |
| | | GATAAGGCAAAAGATACTGTTTATCTCCAGATGAACTCACTCAAGCCCGAAGAC |
| | | ACTGGCATGTACTCCTGCAAGACCAAAGACGGCACCATTGCCACTATGGAGCTG |
| | | TGCGATTTTGGCTACTGGGGTCAGGGCACCCAGGTCACCGTGTCATCC |
| hIL27 | 1522 | CAGGTGCAGGTTCAGGAGAGTGGTGGGGGTCTGGTTCAGCCTGGGGGTAGCCTG |
| Ra_VH | | AGGCTGAGTTGTGCCGCGAGCGGATTTACTTTCTCTCATAGCGGCATGAGTTGG |
| H24- | | GTGCGCCAGGCCCCTGGCAAGGGTCTGGAATGGGTGAGTACAATCAACTCTGGC |
| DR592 | | GGTGCCTCTACCTATTACACCGACAGCGTGAAGGGGAGATTCACTATTAGCCGC |
| | | GACAATGCTAAGAATATGCCCTATCTTCAGCTGAACTCCCTGAAAACCGAAGAC |
| | | ACCGCAATGTATACTGTGCAAAAGGGTGGAAGCGGCTACGGCGACGCCAGCCGC |
| | | ATGACTTCCCCCGGCTCTCAGGGAACTCAGGTTACCGTCTCTTCGGGGGGTAGC |
| | | GGGGGCTCTGGAGGCTCTGGCCAGGTTCAGTTGCAGGAAAGTGGAGGCGGTTCA |
| | | GTTCAGGCTGGAGGCAGCTTGCGCTTGTCCTGTACCGCGCCAGGGTTCACAAGT |
| | | AATAGCTGTGGCATGGATTGGTATCGTCAGGCACCGGGAAAGGAGCGCGAGTTT |
| | | GTGTCTAGCATCAGCACAGAGGGGACAACCGGCTATGCGGATTCCGTAAAAGGA |
| | | CGGTTTACTATCTCCAAGGATAAGGCTAAGGACACAGTGTACCTGCAAATGAAC |
| | | AGCCTGAAGCCCGAAGATACTGGTATGTATAGGTGCAAGACCAAGGATGGCACA |
| | | ATCGCCACTATGGAGCTGTGTGACTTCGGCTATTGGGGTCAGGGCACCCAGGTG |
| | | ACAGTGTCTTCT |
| hIL27 | 1523 | CAGGTGCAGCTCCAGGAAAGCGGAGGTGGCCTGGTACAGCCCGGCGGTAGTCTG |
| Ra_VH | | AGGCTGAGTTGTGCTGCCTCAGGTTTTACTTTCTCACACCCGCGGATGTCCTGG |
| H24- | | GTGAGACAAGCGCCGGGCAAGGGCCTGGAGTGGGTATCAACTATTAACAGTGGC |
| DR593 | | GGGGCGTTCTACCTATTACACTGACAGCGTGAAGGGCCGGTCACTATCAGCAGA |
| | | GATAACGCTAAAAATATGTTGTATCCTCAGCTCAATTCCCTGAAAACCGAAGAT |
| | | ACCGCGATGTATTACTGTGCTAAAGGGGGTTCTGGATACGGCGATGCGTCTCGC |
| | | ATGACCTCTCCTGGAAGCCAGGGCACCCAGGTCACCGTGTCTAGTGGCGGAGGT |
| | | AGTCAGGTTCAGCTCCAAGAGTCCGGCGGAGGTAGCGTCCAGGCCGGGGGGTCA |
| | | CTCCGCCTGTCCTGCGCAGCCAGCGGATACCCCTATTCAAACGGCTACATGGGG |
| | | TGGTTCCGCCAGGGCGGAAAAGAGCCGTGAAGGAGTGGCCTACAATCTCATACC |
| | | GGCGACGGGAGGACCTATTACGCAGATTCCGTGAAAGGCCGTTTCACCATCTCC |
| | | CGTGACAACGCCAAAAATACCGTGGACCTTCAGATGAGTTCTCTGAAGCCAGAG |
| | | GACACCGCTATGTACTATTGTGCTGCCAGAGCTGCACCACTGTATTCCTCTGGC |
| | | AGCCCCCTGACCAGAGCCCGCTACAACGTCTGGGGACAGGGCACCCAGGTCACA |
| | | GTCAGCTCT |
| hIL27 | 1524 | CAGGTTCAGCTCCAGGAATCTGGGGGTGGCCTGGTTCAGCCGGGTGGCAGTCTT |
| Ra_VH | | CGTCTGAGCTGTGCAGCCTCTGGCTTCACCTTCTCTCACTCCGGCATGAGGTGG |
| H24- | | GTCAGACAGGCCCCAGGCAAGGGTCTGGAGTGGGTGTCTACGATCAATAGCGGA |
| DR593 | | GGCGCTTCTACCTATTACACCGACAGCGTGAAGGGCAGATTTACCATTTCCCGC |
| | | GACAACGCCAAAAACATGCCGTATCTGCAACTGAACTCTCTGAAGACCGAGGAC |
| | | ACCGCCATGTACTATTGCGCTAAGGGCGGTAGCGGATATGGCGACGCGAGCAGA |
| | | ATGACTTCTCCGGGGAGCCAGGGTACACAGGTGACCGTGTCCAGCGGGGGTAGT |
| | | GGGGGCAGCGGTGGCAGCGGACAGGTCCAGTTGCAGGAATCCGGGGGAGGCAGC |
| | | GTGCAGGCTGGCGGTTCACTGAGACTCTCTTGCGCGGCCAGCGGCTATCCCTAT |
| | | TCTAACGGTTATATGGGCTGGTTTAGGCAGGCTCCCGGCAAGGAAAGGGAGGGC |
| | | GTGGCTACTATCTATACTGGCGATGGCCGCACTTACTATGCCGATAGTGTCAAG |
| | | GGCCGTTTCACTATCTCTCGCGACAACGCTAAGAACACAGTGGATCTTCAGATG |
| | | TCTTCCCTGAAGCCAGAGGATACCGCGATGTATTACTGTGCCGCGAGGGGTGCA |
| | | CCACTGTACTCTAGCGGATCTCCCCTGACCCGCGCCAGATACAACGTGTGGGGC |
| | | CAGGGGACGCAGGTTACCGTTTCTTCT |
| hIL27 | 1525 | CAGGTGCAGTTGCAGGAGAGTGGAGGCGGACTGGTGCAGCCAGGAGGTTCACTG |
| Ra_VH | | CGTCTTTCTTGCGCAGCCAGCGGTTTTACATTCAGCCACTCTGGAATGTCTTGG |
| H24- | | GTTCGCCAGGCACCGGGGAAGGGCCTGGAGTGGGTGTCCACAATCAACAGTGGG |
| DR594 | | GGTGCCTCTACTTACTATACAGACTCCGTGAAAGGCAGGTTTACCATCTCCAGG |
| | | GACAACGCCAAAAATATGCTTTATCTTCAGCTCAATTCTCTGAAGACCGAGGAT |
| | | ACTGCGATGTATTACTGTGCAAAAGGGGGCTCAGGATATGGCGCATGCCCCCGC |
| | | ATGACCAGCCCAGGGAGCCAGGGCACCCAGGTGACCGTGTCCAGTGGGGGAGGC |
| | | TCTCAGGTGCAGCTCCAGGAGTCAGGCGGTGGCTCCGTCCAGGCTGGCGGGAGG |
| | | TTGCGCTTGTCCTGTGTTGCCTCCGCTAGTACCTATTGCACCTATGATATGCAC |
| | | TGGTACAGACAGGCTCCGGGTAAGGGCCGTGAGTTCGTGTCCGCCATTGATAGC |
| | | GACGGCACAACTCGCTACGCTGATTCCGTGAAAGGCCGGTTCACCATTAGCCAG |
| | | GGCACCGCTAAGAACACAGTGTACTTGCAGATGAACAGCTTGCAGCCAGAGGAT |
| | | ACCGCGATGTATTACTGCAAAACTGTGTGCGTAGTGGGCAGTAGATGGTCCGAC |
| | | TACTGGGGCCAGGGCACACAGGTCACCGTGAGCAGC |
| hIL27 | 1526 | CAGGTACAGCTCCAAGAGTCTGGCGGTGGCCTGGTGCAACCAGGGGGCTCATTG |
| Ra_VH | | AGATTGTCTTGCGCCGCTTCTGGATTCACATTCTCCCACTCAGGTATGAGTTGG |
| H24- | | GTGCGGCAAGCCCCAGGCAAGGGCCTGGAGTGGGTTTCTACCATCAACAGTGGG |
| DR594 | | GGAGCTAGTACCTACTATACAGACTCCGTGAAAGGCCGGTTCACCATTAGCCGC |
| | | GATAACGCTAAGAACATGCTGTACTTGCAGCTCAATTCCCTGAAGACCGAGGAC |
| | | ACAGCTATGTACTATTGCGCCAAGGGAGGCAGTGGTTACGGGGACGCCAGCCGG |
| | | ATGACCTCTCCCGGCTCTCAAGGGACGCAGGTTACCGTGTCAAGCGGCGGTTCC |
| | | GGTGGGTCCGGTGGCTCCGGTCAAGTGCAGCTCCAGGAGAGTGGGGGTGGCAGC |
| | | GTCCAGGCCGGAGGCTCCCTCAGGCTGTCCTGCGTGGCCTCTGCCAGCACATAT |
| | | TGCACATACGACATGCACTGGTATCGTCAGGCTCCTGGCAAAGGGCGCGAGTTC |
| | | GTGTCCGCTATTGATTCCGATGGTACAACCCGGTACGCTGACAGTGTGAAGGGC |
| | | CGTTTCACAATTAGCCAGGGCACCGCTAAAAACACCGTGTACCTCCAGATGAAC |
| | | TCCCTCCAACCTGAGGATACTGCCATGTATTACTGCAAAACGGTGTGCGTCGTG |
| | | GGCTCTCGCTGGTCCGATTACTGGGGGCAGGGCACTCAGGTGACCGTGTCCAGC |
| hIL27 | 1527 | CAGGTGCAGTTGCAGGAGAGCGGAGGCGGGCTGGTGCAGCCTGGAGGCTCTCTG |
| Ra_VH | | CGCCTGTCCTGCGCAGCCTCAGGATTTACATTGTCTCACAGTGGGATGTCTTGG |
| H24- | | GTGCGGCAGGCTCCGGGCAAAGGCCTGGAATGGGTGTCCACCATTAACTCTGGT |
| DR595 | | GGCGCGAGCACTTACTATACCGATAGTGTCAAAGGCCGGTTCACCATCAGCCGG |
| | | GACAACGCTAAGAACATGCTGTATCTCCAGCTGAACAGCCTGAAGACCGAAGAC |
| | | ACTGCTATGTATTACTGTGCCAAGGGCGGTTCAGGTTATGGGGACGCCTCTCGG |
| | | ATGACATCACCGGGCTCCCAAGGAACTCAGGTCACCGTCAGCAGTGGCGGTGGC |
| | | TCCCAGGTGCAACTTCAGGAGTCCGGGGGTGGAAGCGTGCAGGCTGGAGGCTCC |
| | | CTGACACTGTCTTGCGCTGCCAGCGAATACGCTTATTCCACCTGTAACATGGGG |
| | | TGGTATCGCCAAGCTCCTGGGAAGGAACGTGAACTGGTCAGTGCGTTCATCTCT |
| | | GATGGGTCCACGTATTACGCTGATTCAGTAAAGGGACGTTTCACAATCACCGGC |
| | | GACAACGCCAAAAACACTGTGTACTTGCAGATGAACAGCCTGAAGCCAGAGGAT |
| | | ACCGCTATCTACTATTGCAGCGCCAACTGTTACCGTCGGCTGCGCAACTACTGG |
| | | GGGCAGGGCACACAAGTCACAGTGTCCAGC |
| hIL27 | 1528 | CAGGTCCAGCTCCAGGAGTCAGGGGGTGGACTTGTTCAACCCGGTGGCTCCCTC |
| Ra_VH | | CGTCTCTCCTGTGCTGCGTCTGGTTTCACCTTCAGCCACTCTGGAATGAGCTGG |
| H24 | | GTTAGACAAGCACCTGGCAAAGGCCTCGAATGGGTCTTCTACCATAACAGCGGC |
| DR595 | | GGAGCCTCCACTTATTACACCGACTCCGTGAAGGGTCGCTTCACGATCAGCAGA |
| | | GACAACGCCAAGAACATGCTGTACCTCCAACTGAATAGCCTGAAAACCGAGGAT |
| | | ACTGCCATGTATTACTGTGCAAAGGGGGGAAGTGGTTACGGAGACGCCAGCAGG |
| | | ATGACATCTCCGGGTTCTCAAGGCACCCAGGTCACCGTGAGTAGCGGCGGATCT |
| | | GGGGGCAGCGGAGGCAGCGGCCAGGTGCAGCTTCAGGAAAGCGGAGGGGGTTCC |
| | | GTTCAGGCCGGGGGCTCACTGACACTGAGTTGCGCGGCCTCCGAATACGCCTAT |
| | | TCTACTTGTAACATGGGCTGGTACAGACAAGCTCCTGGGAAGGAGAGAGAACTG |
| | | GTGAGCGCTTTTATTTCTGACGGCTCAACCTATTACGCAGACTCCGTCAAGGGC |
| | | CGCTTCACCATTACCGGCGACAACGCTAAGAATACGGTGTACCTCCAGATGAAC |
| | | TCCCTGAAGCCCGAAGACACCGCAATTTACTATTGCAGCGCCAACTGCTACCGG |
| | | CGGTTGCGCAACTACTGGGGCCAGGGAACTCAGGTTACCGTATCCTCC |
| hIL27 | 1529 | CAGGTGCAACTCCAGGAGTCCGGCGGTGGACTGGTGCAGCCTGGCGGTAGCTTG |
| Ra_VH | | AGGCTGTCTTGCGCCGCATCCGGCTTCACATTCAGCCACTCTGGAATGTCATGG |
| H24- | | GTCAGACAGGCTCCGGGCAAGGGCCTGGAATGGGTCTCAACCATCAATAGTGGA |
| DR596 | | GGTGCCTCAACTTACTATACCGACTCCGTGAAAGGTCGTTTCACAATTTCACGC |
| | | GACAACGCTAAGAACATGCTGTACCTCCAGCTGAATAGTCTCAAAACTGAGGAC |
| | | ACCGCTATGTATTACTGTGCTAAGGGCGGTTCCGGGTATGGGGACGCCTCCAGG |
| | | ATGACTTCACCGGGTAGCCAGGGGACCCAGGTGACGGTTTCTAGCGGGGGGGGG |
| | | TCCCAGGTGCAGCTTCAGGAAAGCGGAGGGGGCCTGGTGCAGCCCGGGGGGTCC |
| | | CTGAGACTGAGCTGCACGGCAAGCGGCCTGACGTTTGATGACTCCGTGATGGGC |
| | | TGGTTCAGACAGGCCCCCGGCAAGGGGCGCGAAGCCGTCTCCTGTATTAGCTCC |
| | | AGCGGGGCCAACGCTTTCTACGCGGACAGCGTCAAAGGCCGCTTCACCATTAGC |
| | | AGAGACAACGCTAAGAACACTCTGTACCTCCAGATGAACAGCCTGAAACCAGAG |
| | | GATACCGCAACGTATTACTGCAAGCGCGGACATGCTTGCGCGGGCTATTACCCA |
| | | ATCCCTTATGACGATTACTGGGGCCAGGGCACTCAGGTGACGGTAAGCTCC |
| hIL27 | 1530 | CAGGTCCAACTCCAGGAGTCCGGCGGTGGCCTGGTTCAGCCAGGAGGTTCCCTG |
| Ra_VH | | CGCCTGTCCTGTGCCGCTTCCGGCTTTACCTTCTCCCATTCCGGCATGAGCTGG |
| H24- | | GTGAGGCAGGCCCCTGGCAAAGGCCTGGAGTGGGTGTCTACCATTAACTCTGGC |
| DR596 | | GGAGCCAGCACATACTATACAGACTCAGTAAAGGGACGGTTCACCATCAGCAGG |
| | | GACAACGCCAAAAACATGCTGTATCTCCAGCTGAACTCTCTCAAGACCGAAGAC |
| | | ACCGCTATGTATTACTGTGCTAAGGGCGGTAGCGGCTATGGGGACGCATCTCGT |
| | | ATGACCTCTCCCGGCTCTCAAGGCACCCAGGTGACTGTGTCTAGTGGTGGGTCT |
| | | GGCGGGTCTGGCGGATCTGGTCAAGTCCAGTTGCAGGAGAGTGGAGGCGGTCTC |
| | | GTCCAGCCAGGCGGAAGCCTGCGCCTGTCTTGTACTGCCAGCGGTTTGACCTTT |
| | | GATGACTCTGTGATGGGCTGGTTTCGTCAGGCCCCCGGCAAGGGCCGTGAGGCC |
| | | GTGTCATGTATCTCATCTTCAGGAGCCAACGCCTTTTATGCCGATAGCGTGAAA |
| | | GGGAGATTCACCATCTCTCGTGATAACGCAAAGAACACCCTGTACCTTCAGATG |
| | | AACTCTCTGAAGCCTGAGGACACTGCTACCTACTATTGTAAGCGTGGGCACGCC |
| | | TGCGCTGGATATTACCCTATCCCTTACGATGACTATTGGGGGCAGGGCACTCAG |
| | | GTGACCGTCAGCAGC |

In some embodiments, a bispecific V_{H}H² comprises:
an anti-gp130 V_{H}H antibody comprising a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 193-198; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 199-204; and a CDR3 having at least 90% (e.g., 91%, 92%, 93%, 94%, 95%, 96%, 97%, 98%, 99%, or 100%) sequence identity, or having 0, 1, 2, or 3 amino acid changesoptionally conservative amino acid changes relative, to the sequence of any one of SEQ ID NOS: 205-210; and
an anti-IL27Rα V_{H}H antibody comprising a CDR1 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 211-217; a CDR2 having at least 90% (e.g., 91%, 92%, 93%, 94%, 95%, 96%, 97%, 98%, 99%, or 100%) sequence identity, or having 0, 1, 2, or 3 amino acid changes, optionally conservative amino acid changes relative, to the sequence of any one of SEQ ID NOS: 218-224; and a CDR3 having at least 90% (e.g., 91%, 92%, 93%, 94%, 95%, 96%, 97%, 98%, 99%, or 100%) sequence identity, or having 0, 1, 2, or 3 amino acid changes optionally conservative amino acid changes relative, to the sequence of any one of SEQ ID NOS: 225-231.

In certain embodiments, a bispecific V_{H}H² described herein comprises an anti-gp130 V_{H}H antibody comprising a CDR1, a CDR2, and a CDR3 and an anti-IL27Rα V_{H}H antibody comprising a CDR1, a CDR2, and a CDR3 as described in each row of Table 1 below. In some embodiments, the CDR1, CDR2, and CDR3 in the anti-gp130 V_{H}H antibody and CDR1, CDR2, and CDR3 in the anti-IL27Rα V_{H}H antibody can each, independently, comprise at least 90% (e.g., 91%, 92%, 93%, 94%, 95%, 96%, 97%, 98%, 99%, or 100%) sequence identity, or have 0, 1, 2, or 3 amino acid changes, optionally conservative amino acid changes, relative to the sequence described in each row of Table 1.

In some embodiments, the bispecific V_{H}H² comprises an anti-gp130 V_{H}H antibody at the N-terminus and an anti-IL27Rα V_{H}H antibody at the C-terminus. In some embodiments, the bispecific V_{H}H² comprises an anti-IL27Rα V_{H}H antibody at the N-terminus and an anti-gp130 V_{H}H antibody at the C-terminus.

TABLE 1

| Anti-gp130 V_{H}H | Anti-gp130 V_{H}H | Anti-gp130 V_{H}H | Anti-IL27Rα V_{H}H | Anti-IL27Rα V_{H}H | Anti-IL27Rα V_{H}H |
|---|---|---|---|---|---|
| CDR1 | CDR2 | CDR3 | CDR1 | CDR2 | CDR3 |
| ALASGYIDSR | AIWPGGGLTVY | GSPRMCPSLEFG | FTFSSYPMS | TISAGGDTTLYA | GYCYRRNY |
| (SEQ ID NO: 193) | ADSVKG (SEQ | FDY (SEQ ID | (SEQ ID NO: 211) | DSVKG (SEQ ID | (SEQ ID NO: 225) |
| | ID NO: 199) | NO: 205) | | NO: 218) | |
| ALASGYIDSR | AIWPGGGLTVY | GSPRMCPSLEFG | FTFSNYAMS | GINVAYGITSYA | HSGTTIPRGFISY |
| (SEQ ID NO: 193) | ADSVKG (SEQ | FDY (SEQ ID | (SEQ ID NO: 212) | DSVKG (SEQ ID | TK (SEQ ID |
| | ID NO: 199) | NO: 205) | | NO: 219) | NO: 226) |
| ALASGYIDSR | AIWPGGGLIVY | GSPRMCPSLEFG | FSFSSYAMK | TISSGGSSTNYA | AIVPTGATME |
| (SEQ ID NO: 193) | ADSVKG (SEQ | FDY (SEQ ID | (SEQ ID NO: 213) | DSVKG (SEQ ID | (SEQ ID NO: 227) |
| | ID NO: 199) | NO: 205) | | NO: 220) | |
| AIASGYIDSR | AIWPGGGLTVY | GSPRMCPSLEFG | FTYSTSNSWMA | AIYTVGGSIFYA | ASGRLRGKWFW |
| (SEQ ID NO: 193) | ADSVKG (SEQ | FDY (SEQ ID | (SEQ ID NO: 214) | DSVRG (SEQ ID | PYEYNY (SEQ ID |
| | ID NO: 199) | NO: 205) | | NO: 221) | NO: 228) |
| AIASGYIDSR | AIWPGGGLTVY | GSPRMCPSLEFG | YVSCDYFLPS | HIDGTGSTSYAA | SCVRGRAISEY |
| (SEQ ID NO: 193) | ADSVKG (SEQ | FDY (SEQ ID | (SEQ ID NO: 215) | SVKG (SEQ ID | (SEQ ID NO: 229) |
| | ID NO: 199) | NO: 205) | | NO: 222) | |
| AIASGYIDSR | AIWPGGGLTVY | GSPRMCPSLEFG | STYSNYCLG | VINWVGGMLYF | ESVSSFSCGGWL |
| (SEQ ID NO: 193) | ADSVKG (SEQ | FDY (SEQ ID | (SEQ ID NO: 216) | ADSVKG (SEQ | TRPDRVPY (SEQ |
| | ID NO: 199) | NO: 205) | | ID NO: 223) | ID NO: 230) |
| AIASGYIDSR | ATWPGGGLTVY | GSPRMCPSLEFG | FTFSLSSMS | AISSGGASTYYT | GGSGYGDASRM |
| (SEQ ID NO: 193) | ADSVKG (SEQ | FDY (SEQ ID | (SEQ ID NO: 217) | DSVKG (SEQ ID | TSP (SEQ ID |
| | ID NO: 199) | NO: 205) | | NO: 224) | NO: 231) |
| FTSNSCGMD | SISTDGTTGYAD | KDGTIATMELC | FTFSSYPMS | TISAGGDTILYA | GYCYRRNY |
| (SEQ ID NO: 194) | SVKG (SEQ ID | DEGY (SEQ ID | (SEQ ID NO: 211) | DSVKG (SEQ ID | (SEQ ID NO: 225) |
| | NO: 200) | NO: 206) | | NO: 218) | |
| FTSNSCGMD | SISTDGTTGYAD | KDGTIATMELC | FTFSNYAMS | GINVAYGITSYA | HSGTTIPRGFISY |
| (SEQ ID NO: 194) | SVKG (SEQ ID | DFGY (SEQ ID | (SEQ ID NO: 212) | DSVKG (SEQ ID | TK (SEQ ID |
| | NO: 200) | NO: 206) | | NO: 219) | NO: 226) |
| FTSNSCGMD | SISTDGTTGYAD | KDGTIATMELC | FSFSSYAMK | TISSGGSSTNYA | AIVPTGATME |
| (SEQ ID NO: 194) | SVKG (SEQ ID | DFGY (SEQ ID | (SEQ ID NO: 213) | DSVKG (SEQ ID | (SEQ ID NO: 227) |
| | NO: 200) | NO: 206) | | NO: 220) | |
| FTSNSCGMD | SISTDGTTGYAD | KDGTIATMELC | FTYSTSNSWMA | AIYTVGGSIFYA | ASGRLRGKWFW |
| (SEQ ID NO: 194) | SVKG (SEQ ID | DFGY (SEQ ID | (SEQ ID NO: 214) | DSVRG (SEQ ID | PYEYNY (SEQ ID |
| | NO: 200) | NO: 206) | | NO: 221) | NO: 228) |
| FTSNSCGMD | SISTDGTTGYAD | KDGTIATMELC | YVSCDYFLPS | IIDGTGSTSYAA | SCVRGRAISEY |
| (SEQ ID NO: 194) | SVKG (SEQ ID | DFGY (SEQ ID | (SEQ ID NO: 215) | SVKG (SEQ ID | (SEQ ID NO: 229) |
| | NO: 200) | NO: 206) | | NO: 222) | |
| FTSNSCGMD | SISTDGTTGYAD | KDGTIATMELC | STYSNYCLG | VINWVGGMLYF | ESVSSFSCGGWL |
| (SEQ ID NO: 194) | SVKG (SEQ ID | DFGY (SEQ ID | (SEQ ID NO: 216) | ADSVKG (SEQ | TRPDRVPY (SEQ |
| | NO: 200) | NO: 206) | | ID NO: 223) | ID NO: 230) |
| FTSNSCGMD | SISTDGTTGYAD | KDGTIATMELC | FTFSLSSMS | AISSGGASTYYT | GGSGYGDASRM |
| (SEQ ID NO: 194) | SVKG (SEQ ID | DEGY (SEQ ID | (SEQ ID NO: 217) | DSVKG (SEQ ID | TSP (SEQ ID |
| | NO: 200) | NO: 206) | | NO: 224) | NO: 231) |
| YPYSNGYMG | TIYTGDGRTYY | RAAPLYSSGSPL | FTFSSYPMS | TISAGGDTTLYA | GYCYRRNY |
| (SEQ ID NO: 195) | ADSVKG (SEQ | TRARYNV (SEQ | (SEQ ID NO: 211) | DSVKG (SEQ ID | (SEQ ID NO: 225) |
| | ID NO: 201) | ID NO: 207) | | NO: 218) | |
| YPYSNGYMG | TIYTGDGRTYY | RAAPLYSSGSPL | FTFSNYAMS | GINVAYGITSYA | HSGTTIPRGFISY |
| (SEQ ID NO: 195) | ADSVKG (SEQ | TRARYNV (SEQ | (SEQ ID NO: 212) | DSVKG (SEQ ID | TK (SEQ ID |
| | ID NO: 201) | ID NO: 207) | | NO: 219) | NO: 226) |
| YPYSNGYMG | TIYTGDGRTYY | RAAPLYSSGSPL | FSFSSYAMK | TISSGGSSTNYA | AIVPTGATME |
| (SEQ ID NO: 195) | ADSVKG (SEQ | TRARYNV (SEQ | (SEQ ID NO: 213) | DSVKG (SEQ ID | (SEQ ID NO: 227) |
| | ID NO: 201) | ID NO: 207) | | NO: 220) | |
| YPYSNGYMG | TIYTGDGRTYY | RAAPLYSSGSPL | FTYSTSNSWMA | AIYTVGGSIFYA | ASGRLRGKWFW |
| (SEQ ID NO: 195) | ADSVKG (SEQ | TRARYNV (SEQ | (SEQ ID NO: 214) | DSVRG (SEQ ID | PYEYNY (SEQ ID |
| | ID NO: 201) | ID NO: 207) | | NO: 221) | NO: 228) |
| YPYSNGYMG | TIYTGDGRTYY | RAAPLYSSGSPL | YVSCDYFLPS | HIDGTGSTSYAA | SCVRGRAISEY |
| (SEQ ID NO: 195) | ADSVKG (SEQ | TRARYNV (SEQ | (SEQ ID NO: 215) | SVKG (SEQ ID | (SEQ ID NO: 229) |
| | ID NO: 201) | ID NO: 207) | | NO: 222) | |
| YPYSNGYMG | TIYTGDGRTYY | RAAPLYSSGSPL | STYSNYCLG | VINWVGGMLYF | ESVSSFSCGGWL |
| (SEQ ID NO: 195) | ADSVKG (SEQ | TRARYNV (SEQ | (SEQ ID NO: 216) | ADSVKG (SEQ | TRPDRVPY (SEQ |
| | ID NO: 201) | ID NO: 207) | | ID NO: 223) | ID NO: 230) |
| YPYSNGYMG | TIYTGDGRTYY | RAAPLYSSGSPL | FTFSLSSMS | AISSGGASTYYT | GGSGYGDASRM |
| (SEQ ID NO: 195) | ADSVKG (SEQ | TRARYNV (SEQ | (SEQ ID NO: 217) | DSVKG (SEQ ID | TSP (SEQ ID |
| | ID NO: 201) | ID NO: 207) | | NO: 224) | NO: 231) |
| STYCTYDMH | AIDSDGTTRYAD | GSRWSDY (SEQ | FTFSSYPMS | TISAGGDTTLYA | GYCYRRNY |
| (SEQ ID NO: 196) | SVKG (SEQ ID | ID NO: 208) | (SEQ ID NO: 211) | DSVKG (SEQ ID | (SEQ ID NO: 225) |
| | NO: 202) | | | NO: 218) | |
| STYCTYDMH | AIDSDGTTRYAD | GSRWSDY (SEQ | FTFSNYAMS | GINVAYGITSYA | HSGTTIPRGFISY |
| (SEQ ID NO: 196) | SVKG (SEQ ID | ID NO: 208) | (SEQ ID NO: 212) | DSVKG (SEQ ID | TK (SEQ ID |
| | NO: 202) | | | NO: 219) | NO: 226) |
| STYCTYDMH | AIDSDGTTRYAD | GSRWSDY (SEQ | FSFSSYAMK | TISSGGSSTNYA | AIVPTGATME |
| (SEQ ID NO: 196) | SVKG (SEQ ID | ID NO: 208) | (SEQ ID NO: 213) | DSVKG (SEQ ID | (SEQ ID NO: 227) |
| | NO: 202) | | | NO: 220) | |
| STYCTYDMH | AIDSDGTTRYAD | GSRWSDY (SEQ | FTYSTSNSWMA | AIYTVGGSIFYA | ASGRLRGKWFW |
| (SEQ ID NO: 196) | SVKG (SEQ ID | ID NO: 208) | (SEQ ID NO: 214) | DSVRG (SEQ ID | PYEYNY (SEQ ID |
| | NO: 202) | | | NO: 221) | NO: 228) |
| STYCTYDMH | AIDSDGTTRYAD | GSRWSDY (SEQ | YVSCDYFLPS | IIDGTGSTSYAA | SCVRGRAISEY |
| (SEQ ID NO: 196) | SVKG (SEQ ID | ID NO: 208) | (SEQ ID NO: 215) | SVKG (SEQ ID | (SEQ ID NO: 229) |
| | NO: 202) | | | NO: 222) | |
| STYCTYDMH | AIDSDGTTRYAD | GSRWSDY (SEQ | STYSNYCLG | VINWVGGMLYF | ESVSSFSCGGWL |
| (SEQ ID NO: 196) | SVKG (SEQ ID | ID NO: 208) | (SEQ ID NO: 216) | ADSVKG (SEQ | TRPDRVPY (SEQ |
| | NO: 202) | | | ID NO: 223) | ID NO: 230) |
| STYCTYDMH | AIDSDGTTRYAD | GSRWSDY (SEQ | FTFSLSSMS | AISSGGASTYYT | GGSGYGDASRM |
| (SEQ ID NO: 196) | SVKG (SEQ ID | ID NO: 208) | (SEQ ID NO: 217) | DSVKG (SEQ ID | TSP (SEQ ID |
| | NO: 202) | | | NO: 224) | NO: 231) |
| YAYSTONMG | AFISDGSTYYAD | NCYRRLRNY | FTFSSYPMS | TISAGGDTTLYA | GYCYRRNY |
| (SEQ ID NO: 197) | SVKG (SEQ ID | (SEQ ID | (SEQ ID NO: 211) | DSVKG (SEQ ID | (SEQ ID NO: 225) |
| | NO: 203) | NO: 209) | | NO: 218) | |
| YAYSTCNMG | AFISDGSTYYAD | NCYRRLRNY | FTFSNYAMS | GINVAYGITSYA | HSGTTIPRGFISY |
| (SEQ ID NO: 197) | SVKG (SEQ ID | (SEQ ID | (SEQ ID NO: 212) | DSVKG (SEQ ID | TK (SEQ ID |
| | NO: 203) | NO: 209) | | NO: 219) | NO: 226) |
| YAYSTCNMG | AFISDGSTYYAD | NCYRRLRNY | FSFSSYAMK | TISSGGSSTNYA | AIVPTGATME |
| (SEQ ID NO: 197) | SVKG (SEQ ID | (SEQ ID | (SEQ ID NO: 213) | DSVKG (SEQ ID | (SEQ ID NO: 227) |
| | NO: 203) | NO: 209) | | NO: 220) | |
| YAYSTCNMG | AFISDGSTYYAD | NCYRRLRNY | FTYSTSNSWMA | AIYTVGGSIFYA | ASGRLRGKWFW |
| (SEQ ID NO: 197) | SVKG (SEQ ID | (SEQ ID | (SEQ ID NO: 214) | DSVRG (SEQ ID | PYEYNY (SEQ ID |
| | NO: 203) | NO: 209) | | NO: 221) | NO: 228) |
| YAYSTCNMG | AFISDGSTYYAD | NCYRRLRNY | YVSCDYFLPS | IIDGTGSTSYAA | SCVRGRAISEY |
| (SEQ ID NO: 197) | SVKG (SEQ ID | (SEQ ID | (SEQ ID NO: 215) | SVKG (SEQ ID | (SEQ ID NO: 229) |
| | NO: 203) | NO: 209) | | NO: 222) | |
| YAYSTONMG | AFISDGSTYYAD | NCYRRLRNY | STYSNYCLG | VINWVGGMLYF | ESVSSFSCGGWL |
| (SEQ ID NO: 197) | SVKG (SEQ ID | (SEQ ID | (SEQ ID NO: 216) | ADSVKG (SEQ | TRPDRVPY (SEQ |
| | NO: 203) | NO: 209) | | ID NO: 223) | ID NO: 230) |
| YAYSTONMG | AFISDGSTYYAD | NCYRRLRNY | FTFSLSSMS | AISSGGASTYYT | GGSGYGDASRM |
| (SEQ ID NO: 197) | SVKG (SEQ ID | (SEQ ID | (SEQ ID NO: 217) | DSVKG (SEQ ID | TSP (SEQ ID |
| | NO: 203) | NO: 209) | | NO: 224) | NO: 231) |
| LTFDDSVMG | CISSSGANAFYA | GHACAGYYPIP | FTFSSYPMS | TISAGGDTTLYA | GYCYRRNY |
| (SEQ ID NO: 198) | DSVKG (SEQ ID | YDDY (SEQ ID | (SEQ ID NO: 211) | DSVKG (SEQ ID | (SEQ ID NO: 225) |
| | NO: 204) | NO: 210) | | NO: 218) | |
| LIFDDSVMG | CISSSGANAFYA | GHACAGYYPIP | FTFSNYAMS | GINVAYGITSYA | HSGTTIPRGFISY |
| (SEQ ID NO: 198) | DSVKG (SEQ ID | YDDY (SEQ ID | (SEQ ID NO: 212) | DSVKG (SEQ ID | TK (SEQ ID |
| | NO: 204) | NO: 210) | | NO: 219) | NO: 226) |
| LTFDDSVMG | CISSSGANAFYA | GHACAGYYPIP | FSFSSYAMK | TISSGGSSTNYA | AIVPTGATME |
| (SEQ ID NO: 198) | DSVKG (SEQ ID | YDDY (SEQ ID | (SEQ ID NO: 213) | DSVKG (SEQ ID | (SEQ ID NO: 227) |
| | NO: 204) | NO: 210) | | NO: 220) | |
| LTFDDSVMG | CISSSGANAFYA | GHACAGYYPIP | FTYSTSNSWMA | AIYTVGGSIFYA | ASGRLRGKWFW |
| (SEQ ID NO: 198) | DSVKG (SEQ ID | YDDY (SEQ ID | (SEQ ID NO: 214) | DSVRG (SEQ ID | PYEYNY (SEQ ID |
| | NO: 204) | NO: 210) | | NO: 221) | NO: 228) |
| LTFDDSVMG | CISSSGANAFYA | GHACAGYYPIP | YVSCDYFLPS | IIDGTGSTSYAA | SCVRGRAISEY |
| (SEQ ID NO: 198) | DSVKG (SEQ ID | YDDY (SEQ ID | (SEQ ID NO: 215) | SVKG (SEQ ID | (SEQ ID NO: 229) |
| | NO: 204) | NO: 210) | | NO: 222) | |
| LIFDDSVMG | CISSSGANAFYA | GHACAGYYPIP | STYSNYCLG | VINWVGGMLYF | ESVSSFSCGGWL |
| (SEQ ID NO: 198) | DSVKG (SEQ ID | YDDY (SEQ ID | (SEQ ID NO: 216) | ADSVKG (SEQ | TRPDRVPY (SEQ |
| | NO: 204) | NO: 210) | | ID NO: 223) | ID NO: 230) |
| LIFDDSVMG | CISSSGANAFYA | GHACAGYYPIP | FTFSLSSMS | AISSGGASTYYT | GGSGYGDASRM |
| (SEQ ID NO: 198) | DSVKG (SEQ ID | YDDY (SEQ ID | (SEQ ID NO: 217) | DSVKG (SEQ ID | TSP (SEQ ID |
| | NO: 204) | NO: 210) | | NO: 224) | NO: 231) |

### Anti-GP130-Linker-Anti-Il27Rα V_{H}H

A bispecific V_{H}H² can contain, from the N-terminus to the C-terminus, a first V_{H}H binding to gp130 (an anti-gp130 V_{H}H antibody), a linker, and a second V_{H}H binding to IL27Rα (an anti-IL27Rα V_{H}H antibody). In other words, the linker joins the C-terminus of the anti-gp130 V_{H}H in the binding protein to the N-terminus of the anti-IL27Rα V_{H}H in the binding protein. In some embodiments, a purification peptide, e.g., a six-histidine peptide ((His)₆(SEQ ID NO: 1531) or His-tag) can be included, or not, in the bispecific V_{H}H².

In certain embodiments, a bispecific V_{H}H² described herein comprises an anti-gp130 V_{H}H antibody comprising a sequence having at least 90% (e.g., 91%, 92%, 93%, 94%, 95%, 96%, 97%, 98%, 99%, or 100%) sequence identity to a sequence of any one of SEQ ID NOS: 232-237; and an anti-IL27Rα V_{H}H antibody comprising a sequence having at least 90% (e.g., 91%, 92%, 93%, 94%, 95%, 96%, 97%, 98%, 99%, or 100%) sequence identity to a sequence of any one of SEQ ID NOS: 238-244.

In certain embodiments, a bispecific V_{H}H² described herein comprises an anti-gp130 V_{H}H antibody and an anti-IL27Rα V_{H}H antibody as described in each row of Table 2A below or Table 1A above. In some embodiments, in each row of Table 2A, the anti-gp130 V_{H}H antibody and the anti-IL27Rα V_{H}H antibody can each, independently, comprise at least 90% (e.g., 91%, 92%, 93%, 94%, 95%, 96%, 97%, 98%, 99%, or 100%) sequence identity to the sequence described in each row of Table 2A. In some embodiments, the bispecific V_{H}H² can comprise a linker (e.g., linkers described in Section IV) between the anti-gp130 V_{H}H antibody and the anti-IL27Rα V_{H}H antibody as described in each row of Table 2A below. In particular embodiments, the linker is GGGS (SEQ ID NO:108) or (GGGS) n (SEQ ID NO: 1532), (GGS)nG (SEQ ID NO: 1533), (GGGGS)n (SEQ ID NO: 1534) as described elsewhere herein. The sequence of the anti-gp130 V_{H}H is N-terminus to the linker and the sequence of the anti-IL27Rα V_{H}H is C-terminus to the linker. Examples of linkers are further described in Section IV below. The CDR sequences in each V_{H}H are underlined.

TABLE 2A

| N-terminal Anti-gp130 V_{H}H | C-terminal Anti-IL27Rα V_{H}H |
|---|---|
| QVQLQESGGGSVQAGGSLRLSCTASGAIASGY | QVQLQESGGGLVQPGGSLRLSCAASGFTFSSY |
| IDSRWCMAWFRQAPGKEREGVAAIWPGGGLT | PMSWVRQAPGKGLEWISTISAGGDTTLYADSV |
| VYADSVKGRFTISRDHAKNTLYLQMNNLKPE | KGRFTSSRDNAKNTLYLQLNSLKTEDAAIYYC |
| DTAMYYCAAGSPRMCPSLEFGFDYWGQGTQV | AKRIDCNSGYCYRRNYWGQGTQVTVS (SEQ |
| TVSS (SEQ ID NO: 232) | ID NO: 238) |
| QVQLQESGGGSVQAGGSLRLSCTASGAIASGY | QVQLQESGGGLVQPGESLRLSCTASGFTFSNY |
| IDSRWCMAWFRQAPGKEREGVAAIWPGGGLT | AMSWVRQAPGKGLEWVSGINVAYGITSYADSV |
| VYADSVEGRETISRDHAKNTLYLQMNNLKPE | KGRFTISRDNIKNTLYLQLNSLKTEDTAIYYC |
| DTAMYYCAAGSPRMCPSLEFGFDYWGQGTQV | VKHSGTTIPRGFISYTKRGQGTQVTVS |
| TVSS (SEQ ID NO: 232) | (SEQ ID NO: 239) |
| QVQLQESGGGSVQAGGSLRLSCTASGAIASGY | QVQLQESGGGLVQPGGSLRLSCAASGFSFSSY |
| IDSRWCMAWFRQAPGKEREGVAAIWPGGGLT | AMKWVRQAPGKGLEWVSTISSGGSSTNYADS |
| VYADSVKGRFTISRDHAKNTLYLQMNNLKPE | VKGRFTISRDNAKNTLYLQLNSLKIEDTAMYY |
| DTAMYYCAAGSPRMCPSLEFGFDYWGQGTQV | CAKAIVPTGATMERGQGTQVTVS (SEQ ID |
| TVSS (SEQ ID NO: 232) | NO: 240) |
| QVQLQESGGGSVQAGGSLRLSCTASGAIASGY | QVQLQESGGGSVQSGGSLRLSCAASGFTYSTS |
| IDSRWCMAWFRQAPGKEREGVAAIWPGGGLI | NSWMAWFRQAPGKEREGVAAIYTVGGSIFYA |
| VYADSVKGRFTISRDHAKNTLYLQMNNLKPE | DSVRGRFTISQDATKNMFYLQMNTLKPEDTA |
| DTAMYYCAAGSPRMCPSLEFGFDYWGQGTQV | MYYCAAASGRLRGKWFWPYEYNYWGQGTQ |
| TVSS (SEQ ID NO: 232) | VTVS (SEQ ID NO: 241) |
| QVQLQESGGGSVQAGGSLRLSCTASGAIASGY | QVQLQESGGGSVQAGGSLRLSCVASGYVSCDY |
| IDSRWCMAWFRQAPGKEREGVAAIWPGGGLT | FLPSWYRQAPGKEREFVSIIDGTGSTSYAASV |
| VYADSVKGRFTISRDHAKNTLYLQMNNLKPE | KGRFTASQDKGKNIAYLQMNTLKPEDTAMYY |
| DTAMYYCAAGSPRMCPSLEFGFDYWGQGTQV | CKASCVRGRAISEYWGQGTQVTVS (SEQ ID |
| TVSS (SEQ ID NO: 232) | NO: 242) |
| QVQLQESGGGSVQAGGSLRLSCTASGAIASGY | QVQLQESGGGSVQAGGSLRLSCRASGSTYSNY |
| IDSRWCMAWFRQAPGKEREGVAAIWPGGGLT | CLGWFRQITGKEREGVAVINWVGGMLYFADS |
| VYADSVKGRFTISRDHAKNTLYLQMNNLKPE | VKGRFTVSQDQAKNTVYLQMNSLKPEDTAMY |
| DTAMYYCAAGSPRMCPSLEFGFDYWGQGTQV | YCAAESVSSFSCGGWLTRPDRVPYWGQGTQV |
| TVSS (SEQ ID NO: 232) | TVS (SEQ ID NO: 243) |
| QVQLQESGGGSVQAGGSLRLSCTASGAIASGY | QVQLQESGGGLVQPGGSLRLSCAASGFTFSLS |
| IDSRWCMAWFRQAPGKEREGVAAIWPGGGLT | SMSWVRQAPGKGLEWVSAISSGGASTYYTDSV |
| VYADSVKGRFTISRDHAKNTLYLQMNNLKPE | KGRFTISRDNAKNMLYLQLNSLKTEDTAMYY |
| DTAMYYCAAGSPRMCPSLEFGFDYWGQGTQV | CAKGGSGYGDASRMTSPGSQGTQVTVS (SEQ |
| TVSS (SEQ ID NO: 232) | ID NO: 244) |
| QVQLQESGGGSVQAGGSLRLSCTAPGFTSNSC | QVQLQESGGGLVQPGGSLRLSCAASGFTFSSY |
| GMDWYRQAPGKEREFVSSISTDGTTGYADSV | PMSWVRQAPGKGLEWISTISAGGDTTLYADSV |
| KGRFTISKDKAKDTVYLQMNSLKPEDIGMYS | KGRFTSSRDNAKNTLYLQLNSLKTEDAAIYYC |
| CKTKDGTIATMELCDFGYWGQGTQVTVSS | AKRIDCNSGYCYRRNYWGQGTQVTVS (SEQ |
| (SEQ ID NO: 233) | ID NO: 238) |
| QVQLQESGGGSVQAGGSLRLSCTAPGFTSNSC | QVQLQESGGGLVQPGESLRLSCTASGFTFSNY |
| GMDWYRQAPGKEREFVSSISTDGTTGYADSV | AMSWVRQAPGKGLEWVSGINVAYGITSYADSV |
| KGRFTISKDKAKDTVYLQMNSLKPEDIGMYS | KGRFTISRDNIKNTLYLQLNSLKTEDTAIYYC |
| CKTKDGTIATMELCDFGYWGQGTQVTVSS | VKHSGTTIPRGFISYTKRGQGTQVTVS |
| (SEQ ID NO: 233) | (SEQ ID NO: 239) |
| QVQLQESGGGSVQAGGSLRLSCTAPGFTSNSC | QVQLQESGGGLVQPGGSLRLSCAASGFSFSSY |
| GMDWYRQAPGKEREFVSSISTDGTTGYADSV | AMKWVRQAPGKGLEWVSTISSGGSSTNYADS |
| KGRFTISKDKAKDTVYLQMNSLKPEDIGMYS | VKGRFTISRDNAKNTLYLQLNSLKIEDTAMYY |
| CKTKDGTIATMELCDFGYWGQGTQVTVSS | CAKAIVPTGATMERGQGTQVTVS (SEQ ID |
| (SEQ ID NO: 233) | NO: 240) |
| QVQLQESGGGSVQAGGSLRLSCTAPGFTSNSC | QVQLQESGGGSVQSGGSLRLSCAASGFTYSTS |
| GMDWYRQAPGKEREFVSSISTDGTTGYADSV | NSWMAWFRQAPGKEREGVAAIYTVGGSIFYA |
| KGRFTISKDKAKDTVYLQMNSLKPEDIGMYS | DSVRGRFTISQDATKNMFYLQMNTLKPEDTA |
| CKTKDGTIATMELCDFGYWGQGTQVTVSS | MYYCAAASGRLRGKWFWPYEYNYWGQGTQ |
| (SEQ ID NO: 233) | VTVS (SEQ ID NO: 241) |
| QVQLQESGGGSVQAGGSLRLSCTAPGFTSNSC | QVQLQESGGGSVQAGGSLRLSCVASGYVSCDY |
| GMDWYRQAPGKEREFVSSISTDGTTGYADSV | FLPSWYRQAPGKEREFVSIIDGTGSTSYAASV |
| KGRFTISKDKAKDTVYLQMNSLKPEDIGMYS | KGRFTASQDKGKNIAYLQMNTLKPEDTAMYY |
| CKTKDGTIATMELCDFGYWGQGTQVTVSS | CKASCVRGRAISEYWGQGTQVTVS (SEQ ID |
| (SEQ ID NO: 233) | NO: 242) |
| QVQLQESGGGSVQAGGSLRLSCTAPGFTSNSC | QVQLQESGGGSVQAGGSLRLSCRASGSTYSNY |
| GMDWYRQAPGKEREFVSSISTDGTTGYADSV | CLGWFRQITGKEREGVAVINWVGGMLYFADS |
| KGRFTISKDKAKDTVYLQMNSLKPEDIGMYS | VKGRFTVSQDQAKNTVYLQMNSLKPEDTAMY |
| CKTKDGTIATMELCDFGYWGQGTQVTVSS | YCAAESVSSFSCGGWLTRPDRVPYWGQGTQV |
| (SEQ ID NO: 233) | TVS (SEQ ID NO: 243) |
| QVQLQESGGGSVQAGGSLRLSCTAPGFTSNSC | QVQLQESGGGLVQPGGSLRLSCAASGFTFSLSS |
| GMDWYRQAPGKEREFVSSISTDGTTGYADSV | MSWVRQAPGKGLEWVSAISSGGASTYYTDSV |
| KGRFTISKDKAKDTVYLQMNSLKPEDIGMYS | KGRFTISRDNAKNMLYLQLNSLKTEDTAMYY |
| CKTKDGTIATMELCDFGYWGQGTQVTVSS | CAKGGSGYGDASRMTSPGSQGTQVTVS (SEQ |
| (SEQ ID NO: 233) | ID NO: 244) |
| QVQLQESGGGSVQAGGSLRLSCAASGYPYSN | QVQLQESGGGLVQPGGSLRLSCAASGFTFSSY |
| GYMGWFRQAPGKEREGVATIYTGDGRTYYAD | PMSWVRQAPGKGLEWISTISAGGDTTLYADSV |
| SVKGRFTISRDNAKNTVDLQMSSLKPEDTAMY | KGRFTSSRDNAKNTLYLQLNSLKTEDAAIYYC |
| YCAARAAPLYSSGSPLTRARYNVWGQGTQVT | AKRIDCNSGYCYRRNYWGQGTQVTVS (SEQ |
| VSS (SEQ ID NO: 234) | ID NO: 238) |
| QVQLQESGGGSVQAGGSLRLSCAASGYPYSN | QVQLQESGGGLVQPGESLRLSCTASGFTFSNY |
| GYMGWFRQAPGKEREGVATIYTGDGRTYYAD | AMSWVRQAPGKGLEWVSGINVAYGITSYADSV |
| SVKGRFTISRDNAKNTVDLQMSSLKPEDTAMY | KGRFTISRDNIKNTLYLQLNSLKTEDTAIYYC |
| YCAARAAPLYSSGSPLTRARYNVWGQGTQVT | VKHSGTTIPRGFISYTKRGQGTQVTVS |
| VSS (SEQ ID NO: 234) | (SEQ ID NO: 239) |
| QVQLQESGGGSVQAGGSLRLSCAASGYPYSN | QVQLQESGGGLVQPGGSLRLSCAASGFSFSSY |
| GYMGWFRQAPGKEREGVATIYTGDGRTYYAD | AMKWVRQAPGKGLEWVSTISSGGSSTNYADS |
| SVKGRFTISRDNAKNTVDLQMSSLKPEDTAMY | VKGRFTISRDNAKNTLYLQLNSLKIEDTAMYY |
| YCAARAAPLYSSGSPLTRARYNVWGQGTQVT | CAKAIVPTGATMERGQGTQVTVS (SEQ ID |
| VSS (SEQ ID NO: 234) | NO: 240) |
| QVQLQESGGGSVQAGGSLRLSCAASGYPYSN | QVQLQESGGGSVQSGGSLRLSCAASGFTYSTS |
| GYMGWFRQAPGKEREGVATIYTGDGRTYYAD | NSWMAWFRQAPGKEREGVAAIYTVGGSIFYA |
| SVKGRFTISRDNAKNTVDLQMSSLKPEDTAMY | DSVRGRFTISQDATKNMFYLQMNTLKPEDTA |
| YCAARAAPLYSSGSPLTRARYNVWGQGTQVT | MYYCAAASGRLRGKWFWPYEYNYWGQGTQ |
| VSS (SEQ ID NO: 234) | VTVS (SEQ ID NO: 241) |
| QVQLQESGGGSVQAGGSLRLSCAASGYPYSN | QVQLQESGGGSVQAGGSLRLSCVASGYVSCDY |
| GYMGWFRQAPGKEREGVATIYTGDGRTYYAD | FLPSWYRQAPGKEREFVSIIDGTGSTSYAASV |
| SVKGRFTISRDNAKNTVDLQMSSLKPEDTAMY | KGRFTASQDKGKNIAYLQMNTLKPEDTAMYY |
| YCAARAAPLYSSGSPLTRARYNVWGQGTQVT | CKASCVRGRAISEYWGQGTQVTVS (SEQ ID |
| VSS (SEQ ID NO: 234) | NO: 242) |
| QVQLQESGGGSVQAGGSLRLSCAASGYPYSN | QVQLQESGGGSVQAGGSLRLSCRASGSTYSNY |
| GYMGWFRQAPGKEREGVATIYTGDGRTYYAD | CLGWFRQITGKEREGVAVINWVGGMLYFADS |
| SVKGRFTISRDNAKNTVDLQMSSLKPEDTAMY | VKGRFTVSQDQAKNTVYLQMNSLKPEDTAMY |
| YCAARAAPLYSSGSPLTRARYNVWGQGTQVT | YCAAESVSSFSCGGWLTRPDRVPYWGQGTQV |
| VSS (SEQ ID NO: 234) | TVS (SEQ ID NO: 243) |
| QVQLQESGGGSVQAGGSLRLSCAASGYPYSN | QVQLQESGGGLVQPGGSLRLSCAASGFTFSLSS |
| GYMGWFRQAPGKEREGVATIYTGDGRTYYAD | MSWVRQAPGKGLEWVSAISSGGASTYYTDSV |
| SVKGRFTISRDNAKNTVDLQMSSLKPEDTAMY | KGRFTISRDNAKNMLYLQLNSLKTEDTAMYY |
| YCAARAAPLYSSGSPLTRARYNVWGQGTQVT | CAKGGSGYGDASRMTSPGSQGTQVTVS (SEQ |
| VSS (SEQ ID NO: 234) | ID NO: 244) |
| QVQLQESGGGSVQAGGSLRLSCVASASTYCTY | QVQLQESGGGLVQPGGSLRLSCAASGFTFSSY |
| DMHWYRQAPGKGREFVSAIDSDGTTRYADSV | PMSWVRQAPGKGLEWISTISAGGDTTLYADSV |
| KGRFTISQGTAKNTVYLQMNSLQPEDTAMYY | KGRFTSSRDNAKNTLYLQLNSLKTEDAAIYYC |
| CKTVCVVGSRWSDYWGQGTQVTVSS (SEQ ID | AKRIDCNSGYCYRRNYWGQGTQVTVS (SEQ |
| NO: 235) | ID NO: 238) |
| QVQLQESGGGSVQAGGSLRLSCVASASTYCTY | QVQLQESGGGLVQPGESLRLSCTASGFTFSNY |
| DMHWYRQAPGKGREFVSAIDSDGTTRYADSV | AMSWVRQAPGKGLEWVSGINVAYGITSYADSV |
| KGRFTISQGTAKNTVYLQMNSLQPEDTAMYY | KGRFTISRDNIKNTLYLQLNSLKTEDTAIYYC |
| CKTVCVVGSRWSDYWGQGTQVTVSS (SEQ ID | VKHSGTTIPRGFISYTKRGQGTQVTVS |
| NO: 235) | (SEQ ID NO: 239) |
| QVQLQESGGGSVQAGGSLRLSCVASASTYCTY | QVQLQESGGGLVQPGGSLRLSCAASGFSFSSY |
| DMHWYRQAPGKGREFVSAIDSDGTTRYADSV | AMKWVRQAPGKGLEWVSTISSGGSSTNYADS |
| KGRFTISQGTAKNTVYLQMNSLQPEDTAMYY | VKGRFTISRDNAKNTLYLQLNSLKIEDTAMYY |
| CKTVCVVGSRWSDYWGQGTQVTVSS (SEQ ID | CAKAIVPTGATMERGQGTQVTVS (SEQ ID |
| NO: 235) | NO: 240) |
| QVQLQESGGGSVQAGGSLRLSCVASASTYCTY | QVQLQESGGGSVQSGGSLRLSCAASGFTYSTS |
| DMHWYRQAPGKGREFVSAIDSDGTTRYADSV | NSWMAWFRQAPGKEREGVAAIYTVGGSIFYA |
| KGRFTISQGTAKNTVYLQMNSLQPEDTAMYY | DSVRGRFTISQDATKNMFYLQMNTLKPEDTA |
| CKTVCVVGSRWSDYWGQGTQVTVSS (SEQ ID | MYYCAAASGRLRGKWFWPYEYNYWGQGTQ |
| NO: 235) | VTVS (SEQ ID NO: 241) |
| QVQLQESGGGSVQAGGSLRLSCVASASTYCTY | QVQLQESGGGSVQAGGSLRLSCVASGYVSCDY |
| DMHWYRQAPGKGREFVSAIDSDGTTRYADSV | FLPSWYRQAPGKEREFVSIIDGTGSTSYAASV |
| KGRFTISQGTAKNTVYLQMNSLQPEDTAMYY | KGRFTASQDKGKNIAYLQMNTLKPEDTAMYY |
| CKTVCVVGSRWSDYWGQGTQVTVSS (SEQ ID | CKASCVRGRAISEYWGQGTQVTVS (SEQ ID |
| NO: 235) | NO: 242) |
| QVQLQESGGGSVQAGGSLRLSCVASASTYCTY | QVQLQESGGGSVQAGGSLRLSCRASGSTYSNY |
| DMHWYRQAPGKGREFVSAIDSDGTTRYADSV | CLGWFRQITGKEREGVAVINWVGGMLYFADS |
| KGRFTISQGTAKNTVYLQMNSLQPEDTAMYY | VKGRFTVSQDQAKNTVYLQMNSLKPEDTAMY |
| CKTVCVVGSRWSDYWGQGTQVTVSS (SEQ ID | YCAAESVSSFSCGGWLTRPDRVPYWGQGTQV |
| NO: 235) | TVS (SEQ ID NO: 243) |
| QVQLQESGGGSVQAGGSLRLSCVASASTYCTY | QVQLQESGGGLVQPGGSLRLSCAASGFTFSLSS |
| DMHWYRQAPGKGREFVSAIDSDGTTRYADSV | MSWVRQAPGKGLEWVSAISSGGASTYYTDSV |
| KGRFTISQGTAKNTVYLQMNSLQPEDTAMYY | KGRFTISRDNAKNMLYLQLNSLKTEDTAMYY |
| CKTVCVVGSRWSDYWGQGTQVTVSS (SEQ ID | CAKGGSGYGDASRMTSPGSQGTQVTVS (SEQ |
| NO: 235) | ID NO: 244) |
| QVQLQESGGGSVQAGGSLTLSCAASEYAYSTC | QVQLQESGGGLVQPGGSLRLSCAASGFTFSSY |
| NMGWYRQAPGKERELVSAFISDGSTYYADSV | PMSWVRQAPGKGLEWISTISAGGDTTLYADSV |
| KGRFTITRDNAKNTVYLQMNSLKPEDTAIYYC | KGRFTSSRDNAKNTLYLQLNSLKTEDAAIYYC |
| SANCYRRLRNYWGQGTQVTVSS (SEQ ID | AKRIDCNSGYCYRRNYWGQGTQVTVS (SEQ |
| NO: 236) | ID NO: 238) |
| QVQLQESGGGSVQAGGSLTLSCAASEYAYSTC | QVQLQESGGGLVQPGESLRLSCTASGFTFSNY |
| NMGWYRQAPGKERELVSAFISDGSTYYADSV | AMSWVRQAPGKGLEWVSGINVAYGITSYADSV |
| KGRFTITRDNAKNTVYLQMNSLKPEDTAIYYC | KGRFTISRDNIKNTLYLQLNSLKTEDTAIYYC |
| SANCYRRLRNYWGQGTQVTVSS (SEQ ID | VKHSGTTIPRGFISYTKRGQGTQVTVS |
| NO: 236) | (SEQ ID NO: 239) |
| QVQLQESGGGSVQAGGSLTLSCAASEYAYSTC | QVQLQESGGGLVQPGGSLRLSCAASGFSFSSY |
| NMGWYRQAPGKERELVSAFISDGSTYYADSV | AMKWVRQAPGKGLEWVSTISSGGSSTNYADS |
| KGRFTITRDNAKNTVYLQMNSLKPEDTAIYYC | VKGRFTISRDNAKNTLYLQLNSLKIEDTAMYY |
| SANCYRRLRNYWGQGTQVTVSS (SEQ ID | CAKAIVPTGATMERGQGTQVTVS (SEQ ID |
| NO: 236) | NO: 240) |
| QVQLQESGGGSVQAGGSLTLSCAASEYAYSTC | QVQLQESGGGSVQSGGSLRLSCAASGFTYSTS |
| NMGWYRQAPGKERELVSAFISDGSTYYADSV | NSWMAWFRQAPGKEREGVAAIYTVGGSIFYA |
| KGRFTITRDNAKNTVYLQMNSLKPEDTAIYYC | DSVRGRFTISQDATKNMFYLQMNTLKPEDTA |
| SANCYRRLRNYWGQGTQVTVSS (SEQ ID | MYYCAAASGRLRGKWFWPYEYNYWGQGTQ |
| NO: 236) | VTVS (SEQ ID NO: 241) |
| QVQLQESGGGSVQAGGSLTLSCAASEYAYSTC | QVQLQESGGGSVQAGGSLRLSCVASGYVSCDY |
| NMGWYRQAPGKERELVSAFISDGSTYYADSV | FLPSWYRQAPGKEREFVSIIDGTGSTSYAASV |
| KGRFTITRDNAKNTVYLQMNSLKPEDTAIYYC | KGRFTASQDKGKNIAYLQMNTLKPEDTAMYY |
| SANCYRRLRNYWGQGTQVTVSS (SEQ ID | CKASCVRGRAISEYWGQGTQVTVS (SEQ ID |
| NO: 236) | NO: 242) |
| QVQLQESGGGSVQAGGSLTLSCAASEYAYSTC | QVQLQESGGGSVQAGGSLRLSCRASGSTYSNY |
| NMGWYRQAPGKERELVSAFISDGSTYYADSV | CLGWFRQITGKEREGVAVINWVGGMLYFADS |
| KGRFTITRDNAKNTVYLQMNSLKPEDTAIYYC | VKGRFTVSQDQAKNTVYLQMNSLKPEDTAMY |
| SANCYRRLRNYWGQGTQVTVSS (SEQ ID | YCAAESVSSFSCGGWLTRPDRVPYWGQGTQV |
| NO: 236) | TVS (SEQ ID NO: 243) |
| QVQLQESGGGSVQAGGSLTLSCAASEYAYSTC | QVQLQESGGGLVQPGGSLRLSCAASGFTFSLSS |
| NMGWYRQAPGKERELVSAFISDGSTYYADSV | MSWVRQAPGKGLEWVSAISSGGASTYYTDSV |
| KGRFTITRDNAKNTVYLQMNSLKPEDTAIYYC | KGRFTISRDNAKNMLYLQLNSLKTEDTAMYY |
| SANCYRRLRNYWGQGTQVTVSS (SEQ ID | CAKGGSGYGDASRMTSPGSQGTQVTVS (SEQ |
| NO: 236) | ID NO: 244) |
| QVQLQESGGGLVQPGGSLRLSCTASGLTFDDS | QVQLQESGGGLVQPGGSLRLSCAASGFTFSSY |
| VMGWFRQAPGKGREAVSCISSSGANAFYADS | PMSWVRQAPGKGLEWISTISAGGDTTLYADSV |
| VKGRFTISRDNAKNTLYLQMNSLKPEDTATYY | KGRFTSSRDNAKNTLYLQLNSLKTEDAAIYYC |
| CKRGHACAGYYPIPYDDYWGQGTQVTVSS | AKRIDCNSGYCYRRNYWGQGTQVTVS (SEQ |
| (SEQ ID NO: 237) | ID NO: 238) |
| QVQLQESGGGLVQPGGSLRLSCTASGLTFDDS | QVQLQESGGGLVQPGESLRLSCTASGFTFSNY |
| VMGWFRQAPGKGREAVSCISSSGANAFYADS | AMSWVRQAPGKGLEWVSGINVAYGITSYADSV |
| VKGRFTISRDNAKNTLYLQMNSLKPEDTATYY | KGRFTISRDNIKNTLYLQLNSLKTEDTAIYYC |
| CKRGHACAGYYPIPYDDYWGQGTQVTVSS | VKHSGTTIPRGFISYTKRGQGTQVTVS |
| (SEQ ID NO: 237) | (SEQ ID NO: 239) |
| QVQLQESGGGLVQPGGSLRLSCTASGLTFDDS | QVQLQESGGGLVQPGGSLRLSCAASGFSFSSY |
| VMGWFRQAPGKGREAVSCISSSGANAFYADS | AMKWVRQAPGKGLEWVSTISSGGSSTNYADS |
| VKGRFTISRDNAKNTLYLQMNSLKPEDTATYY | VKGRFTISRDNAKNTLYLQLNSLKIEDTAMYY |
| CKRGHACAGYYPIPYDDYWGQGTQVTVSS | CAKAIVPTGATMERGQGTQVTVS (SEQ ID |
| (SEQ ID NO: 237) | NO: 240) |
| QVQLQESGGGLVQPGGSLRLSCTASGLTFDDS | QVQLQESGGGSVQSGGSLRLSCAASGFTYSTS |
| VMGWFRQAPGKGREAVSCISSSGANAFYADS | NSWMAWFRQAPGKEREGVAAIYTVGGSIFYA |
| VKGRFTISRDNAKNTLYLQMNSLKPEDTATYY | DSVRGRFTISQDATKNMFYLQMNTLKPEDTA |
| CKRGHACAGYYPIPYDDYWGQGTQVTVSS | MYYCAAASGRLRGKWFWPYEYNYWGQGTQ |
| (SEQ ID NO: 237) | VTVS (SEQ ID NO: 241) |
| QVQLQESGGGLVQPGGSLRLSCTASGLTFDDS | QVQLQESGGGSVQAGGSLRLSCVASGYVSCDY |
| VMGWFRQAPGKGREAVSCISSSGANAFYADS | FLPSWYRQAPGKEREFVSIIDGTGSTSYAASV |
| VKGRFTISRDNAKNTLYLQMNSLKPEDTATYY | KGRFTASQDKGKNIAYLQMNTLKPEDTAMYY |
| CKRGHACAGYYPIPYDDYWGQGTQVTVSS | CKASCVRGRAISEYWGQGTQVTVS (SEQ ID |
| (SEQ ID NO: 237) | NO: 242) |
| QVQLQESGGGLVQPGGSLRLSCTASGLTFDDS | QVQLQESGGGSVQAGGSLRLSCRASGSTYSNY |
| VMGWFRQAPGKGREAVSCISSSGANAFYADS | CLGWFRQITGKEREGVAVINWVGGMLYFADS |
| VKGRFTISRDNAKNTLYLQMNSLKPEDTATYY | VKGRFTVSQDQAKNTVYLQMNSLKPEDTAMY |
| CKRGHACAGYYPIPYDDYWGQGTQVTVSS | YCAAESVSSFSCGGWLTRPDRVPYWGQGTQV |
| (SEQ ID NO: 237) | TVS (SEQ ID NO: 243) |
| QVQLQESGGGLVQPGGSLRLSCTASGLTFDDS | QVQLQESGGGLVQPGGSLRLSCAASGFTFSLSS |
| VMGWFRQAPGKGREAVSCISSSGANAFYADS | MSWVRQAPGKGLEWVSAISSGGASTYYTDSV |
| VKGRFTISRDNAKNTLYLQMNSLKPEDTATYY | KGRFTISRDNAKNMLYLQLNSLKTEDTAMYY |
| CKRGHACAGYYPIPYDDYWGQGTQVTVSS | CAKGGSGYGDASRMTSPGSQGTQVTVS (SEQ |
| (SEQ ID NO: 237) | ID NO: 244) |

In certain embodiments, the bispecific V_{H}H² comprises a sequence that is substantially identical to a sequence of any one of SEQ ID NOS: 1-42. Such a bispecific V_{H}H² can have a sequence having at least 90% (e.g., 91%, 92%, 93%, 94%, 95%, 96%, 97%, 98%, 99%, or 100%) sequence identity to a sequence of any one of SEQ ID NOS: 1-42, as shown in Table 2B below. In each of sequences of SEQ ID NOS: 1-42, the linker GGGS (SEQ ID NO: 108) is in bold. The sequence of the anti-gp130 V_{H}H is N-terminus to the linker and the sequence of the anti-IL27Rα V_{H}H is C-terminus to the linker. The CDR sequences in each V_{H}H are underlined.

TABLE 2B

| SEQ | |
|---|---|
| ID | |
| NO | Sequence |
| 1 | QVQLQESGGGSVQAGGSLRLSCTASGAIASGYIDSRWCMAWFRQAPGKEREGVAAIWPGG |
| | GLIVYADSVKGRFTISRDHAKNTLYLQMNNLKPEDTAMYYCAAGSPRMCPSLEFGFDYWG |
| | QGTQVTVSSGGGSQVQLQESGGGLVQPGGSLRLSCAASGFTFSSYPMSWVRQAPGKGLEW |
| | ISTISAGGDTILYADSVKGRFTSSRDNAKNTLYLQLNSLKTEDAAIYYCAKRIDCNSGYC |
| | YRRNYWGQGTQVTVSS |
| 2 | QVQLQESGGGSVQAGGSLRLSCTASGAIASGYIDSRWCMAWFRQAPGKEREGVAAIWPGG |
| | GLTVYADSVKGRFTISRDHAKNTLYLQMNNLKPEDTAMYYCAAGSPRMCPSLEFGFDYW |
| | GQGTQVTVSSGGGSQVQLQESGGGLVQPGESLRLSCTASGFTFSNYAMSWVRQAPGKGLE |
| | WVSGINVAYGITSYADSVKGRFTISRDNTKNTLYLQLNSLKTEDTAIYYCVKHSGTTIPR |
| | GFISYTKRGQGTQVTVSS |
| 3 | QVQLQESGGGSVQAGGSLRLSCTASGAIASGYIDSRWCMAWFRQAPGKEREGVAAIWPGG |
| | GLTVYADSVKGRFTISRDHAKNTLYLQMNNLKPEDTAMYYCAAGSPRMCPSLEFGFDYW |
| | GQGTQVTVSSGGGSQVQLQESGGGLVQPGGSLRLSCAASGFSFSSYAMKWVRQAPGKGL |
| | EWVSTISSGGSSTNYADSVKGRFTISRDNAKNILYLQLNSLKIEDTAMYYCAKAIVPTG |
| | ATMERGQGTQVTVSS |
| 4 | QVQLQESGGGSVQAGGSLRLSCTASGAIASGYIDSRWCMAWFRQAPGKEREGVAAIWPGG |
| | GLTVYADSVKGRFTISRDHAKNTLYLQMNNLKPEDTAMYYCAAGSPRMCPSLEFGFDYW |
| | GQGTQVTVSSGGGSQVQLQESGGGSVQSGGSLRLSCAASGFTYSTSNSWMAWFRQAPGK |
| | EREGVAAIYTVGGSIFYADSVRGRFTISQDATKNMFYLQMNTLKPEDTAMYYCAAASGRL |
| | RGKWFWPYEYNYWGQGTQVTVSS |
| 5 | QVQLQESGGGSVQAGGSLRLSCTASGAIASGYIDSRWCMAWFRQAPGKEREGVAAIWPGG |
| | GLTVYADSVKGRFTISRDHAKNTLYLQMNNLKPEDTAMYYCAAGSPRMCPSLEFGFDYW |
| | GQGTQVTVSSGGGSQVQLQESGGGSVQAGGSLRLSCVASGYVSCDYFLPSWYRQAPGKE |
| | REFVSIIDGTGSTSYAASVKGRFTASQDKGKNIAYLQMNTLKPEDTAMYYCKASCVRGR |
| | AISEYWGQGTQVTVSS |
| 6 | QVQLQESGGGSVQAGGSLRLSCTASGAIASGYIDSRWCMAWFRQAPGKEREGVAAIWPGG |
| | GLTVYADSVKGRFTISRDHAKNTLYLQMNNLKPEDTAMYYCAAGSPRMCPSLEFGFDYW |
| | GQGTQVTVSSGGGSQVQLQESGGGSVQAGGSLRLSCRASGSTYSNYCLGWFRQITGKERE |
| | GVAVINWVGGMLYFADSVKGRFTVSQDQAKNTVYLQMNSLKPEDTAMYYCAAESVSSFS |
| | CGGWLTRPDRVPYWGQGTQVTVSS |
| 7 | QVQLQESGGGSVQAGGSLRLSCTASGAIASGYIDSRWCMAWFRQAPGKEREGVAAIWPGG |
| | GLTVYADSVKGRFTISRDHAKNTLYLQMNNLKPEDTAMYYCAAGSPRMCPSLEFGFDYW |
| | GQGTQVTVSSGGGSQVQLQESGGGLVQPGGSLRLSCAASGFTFSLSSMSWVRQAPGKGLE |
| | WVSAISSGGASTYYTDSVKGRFTISRDNAKNMLYLQLNSLKTEDTAMYYCAKGGSGYGDA |
| | SRMTSPGSQGTQVTVSS |
| 8 | QVQLQESGGGSVQAGGSLRLSCTAPGFTSNSCGMDWYRQAPGKEREFVSSISTDGTTGYA |
| | DSVKGRFTISKDKAKDTVYLQMNSLKPEDIGMYSCKTKDGTIATMELCDFGYWGQGTQVT |
| | VSSGGGSQVQLQESGGGLVQPGGSLRLSCAASGFTFSSYPMSWVRQAPGKGLEWISTISA |
| | GGDTTLYADSVKGRFTSSRDNAKNTLYLQLNSLKTEDAAIYYCAKRIDCNSGYCYRRNYW |
| | GQGTQVTVSS |
| 9 | QVQLQESGGGSVQAGGSLRLSCTAPGFTSNSCGMDWYRQAPGKEREFVSSISTDGTTGYA |
| | DSVKGRFTISKDKAKDTVYLQMNSLKPEDIGMYSCKTKDGTIATMELCDFGYWGQGTQV |
| | TVSSGGGSQVQLQESGGGLVQPGESLRLSCTASGFTFSNYAMSWVRQAPGKGLEWVSGIN |
| | VAYGITSYADSVKGRFTISRDNIKNILYLQLNSLKTEDTAIYYCVKHSGTTIPRGFISYT |
| | KRGQGTQVTVSS |
| 10 | QVQLQESGGGSVQAGGSLRLSCTAPGFTSNSCGMDWYRQAPGKEREFVSSISTDGTTGYA |
| | DSVKGRFTISKDKAKDTVYLQMNSLKPEDIGMYSCKTKDGTIATMELCDFGYWGQGTQV |
| | TVSSGGGSQVQLQESGGGLVQPGGSLRLSCAASGFSFSSYAMKWVRQAPGKGLEWVSTIS |
| | SGGSSTNYADSVKGRFTISRDNAKNTLYLQLNSLKIEDTAMYYCAKAIVPTGATMERGQGT |
| | QVTVSS |
| 11 | QVQLQESGGGSVQAGGSLRLSCTAPGFTSNSCGMDWYRQAPGKEREFVSSISTDGTTGYA |
| | DSVKGRFTISKDKAKDTVYLQMNSLKPEDIGMYSCKTKDGTIATMELCDFGYWGQGTQV |
| | TVSSGGGSQVQLQESGGGSVQSGGSLRLSCAASGFTYSTSNSWMAWFRQAPGKEREGVA |
| | AIYTVGGSIFYADSVRGRFTISQDATKNMFYLQMNTLKPEDTAMYYCAAASGRLRGKWFW |
| | PYEYNYWGQGTQVTVSS |
| 12 | QVQLQESGGGSVQAGGSLRLSCTAPGFTSNSCGMDWYRQAPGKEREFVSSISTDGTTGYA |
| | DSVKGRFTISKDKAKDTVYLQMNSLKPEDIGMYSCKTKDGTIATMELCDFGYWGQGTQVT |
| | VSSGGGSQVQLQESGGGSVQAGGSLRLSCVASGYVSCDYFLPSWYRQAPGKEREFVSIID |
| | GTGSTSYAASVKGRFTASQDKGKNIAYLQMNTLKPEDTAMYYCKASCVRGRAISEYWGQ |
| | GTQVTVSS |
| 13 | QVQLQESGGGSVQAGGSLRLSCTAPGFTSNSCGMDWYRQAPGKEREFVSSISTDGTTGYA |
| | DSVKGRFTISKDKAKDTVYLQMNSLKPEDIGMYSCKTKDGTIATMELCDFGYWGQGTQVT |
| | VSSGGGSQVQLQESGGGSVQAGGSLRLSCRASGSTYSNYCLGWFRQITGKEREGVAVIN |
| | WVGGMLYFADSVKGRFTVSQDQAKNTVYLQMNSLKPEDTAMYYCAAESVSSFSCGGWL |
| | TRPDRVPYWGQGTQVTVSS |
| 14 | QVQLQESGGGSVQAGGSLRLSCTAPGFTSNSCGMDWYRQAPGKEREFVSSISTDGTTGYA |
| | DSVKGRFTISKDKAKDTVYLQMNSLKPEDTGMYSCKTKDGTIATMELCDFGYWGQGTQVT |
| | VSSGGGSQVQLQESGGGLVQPGGSLRLSCAASGFTFSLSSMSWVRQAPGKGLEWVSAISS |
| | GGASTYYTDSVKGRFTISRDNAKNMLYLQLNSLKTEDTAMYYCAKGGSGYGDASRMTSP |
| | GSQGTQVTVSS |
| 15 | QVQLQESGGGSVQAGGSLRLSCAASGYPYSNGYMGWFRQAPGKEREGVATIYTGDGRTY |
| | YADSVKGRFTISRDNAKNTVDLQMSSLKPEDTAMYYCAARAAPLYSSGSPLTRARYNVWG |
| | QGTQVTVSSGGGSQVQLQESGGGLVQPGGSLRLSCAASGFTFSSYPMSWVRQAPGKGLEW |
| | ISTISAGGDTTLYADSVKGRFTSSRDNAKNTLYLQLNSLKTEDAAIYYCAKRIDQNSGYC |
| | YRRNYWGQGTQVTVSS |
| 16 | QVQLQESGGGSVQAGGSLRLSCAASGYPYSNGYMGWFRQAPGKEREGVATIYTGDGRTY |
| | YADSVKGRFTISRDNAKNTVDLQMSSLKPEDTAMYYCAARAAPLYSSGSPLTRARYNVWG |
| | QGTQVTVSSGGGSQVQLQESGGGLVQPGESLRLSCTASGFTFSNYAMSWVRQAPGKGLE |
| | WVSGINVAYGITSYADSVKGRFTISRDNTKNTLYLQLNSLKTEDTAIYYCVKHSGTTIPR |
| | GFISYTKRGQGTQVTVSS |
| 17 | QVQLQESGGGSVQAGGSLRLSCAASGYPYSNGYMGWFRQAPGKEREGVATIYTGDGRTY |
| | YADSVKGRFTISRDNAKNTVDLQMSSLKPEDTAMYYCAARAAPLYSSGSPLTRARYNVWG |
| | QGTQVTVSSGGGSQVQLQESGGGLVQPGGSLRLSCAASGFSFSSYAMKWVRQAPGKGLE |
| | WVSTISSGGSSTNYADSVKGRFTISRDNAKNTLYLQLNSLKIEDTAMYYCAKAIVPTGA |
| | TMERGQGTQVTVSS |
| 18 | QVQLQESGGGSVQAGGSLRLSCAASGYPYSNGYMGWFRQAPGKEREGVATIYTGDGRTY |
| | YADSVKGRFTISRDNAKNTVDLQMSSLKPEDTAMYYCAARAAPLYSSGSPLTRARYNVWG |
| | QGTQVTVSSGGGSQVQLQESGGGSVQSGGSLRLSCAASGFTYSTSNSWMAWFRQAPGKE |
| | REGVAAIYTVGGSIFYADSVRGRFTISQDATKNMFYLQMNTLKPEDTAMYYCAAASGRLR |
| | GKWFWPYEYNYWGQGTQVTVSS |
| 19 | QVQLQESGGGSVQAGGSLRLSCAASGYPYSNGYMGWFRQAPGKEREGVATIYTGDGRTY |
| | YADSVKGRFTISRDNAKNTVDLQMSSLKPEDTAMYYCAARAAPLYSSGSPLTRARYNVWG |
| | QGTQVTVSSGGGSQVQLQESGGGSVQAGGSLRLSCVASGYVSCDYFLPSWYRQAPGKERE |
| | FVSIIDGTGSTSYAASVKGRFTASQDKGKNIAYLQMNTLKPEDTAMYYCKASCVRGRAISE |
| | YWGQGTQVTVSS |
| 20 | QVQLQESGGGSVQAGGSLRLSCAASGYPYSNGYMGWFRQAPGKEREGVATIYTGDGRTY |
| | YADSVKGRFTISRDNAKNTVDLQMSSLKPEDTAMYYCAARAAPLYSSGSPLTRARYNVWG |
| | QGTQVTVSSGGGSQVQLQESGGGSVQAGGSLRLSCRASGSTYSNYCLGWFRQITGKEREG |
| | VAVINWVGGMLYFADSVKGRFTVSQDQAKNTVYLQMNSLKPEDTAMYYCAAESVSSESC |
| | GGWLTRPDRVPYWGQGTQVTVSS |
| 21 | QVQLQESGGGSVQAGGSLRLSCAASGYPYSNGYMGWFRQAPGKEREGVATIYTGDGRTY |
| | YADSVKGRFTISRDNAKNTVDLQMSSLKPEDTAMYYCAARAAPLYSSGSPLTRARYNVWG |
| | QGTQVTVSSGGGSQVQLQESGGGLVQPGGSLRLSCAASGFTFSLSSMSWVRQAPGKGLEW |
| | VSAISSGGASTYYTDSVKGRFTISRDNAKNMLYLQLNSLKTEDTAMYYCAKGGSGYGDAS |
| | RMTSPGSQGTQVTVSS |
| 22 | QVQLQESGGGSVQAGGSLRLSCVASASTYCTYDMHWYRQAPGKGREFVSAIDSDGTTRYA |
| | DSVKGRFTISQGTAKNTVYLQMNSLQPEDTAMYYCKTVCVVGSRWSDYWGQGTQVTVSS |
| | GGGSQVQLQESGGGLVQPGGSLRLSCAASGFTFSSYPMSWVRQAPGKGLEWISTISAGGDT |
| | TLYADSVKGRFTSSRDNAKNTLYLQLNSLKTEDAAIYYCAKRIDCNSGYCYRRNYWGQGT |
| | QVTVSS |
| 23 | QVQLQESGGGSVQAGGSLRLSCVASASTYCTYDMHWYRQAPGKGREFVSAIDSDGTTRYA |
| | DSVKGRFTISQGTAKNTVYLQMNSLQPEDTAMYYCKTVCVVGSRWSDYWGQGTQVTVSS |
| | GGGSQVQLQESGGGLVQPGESLRLSCTASGFTFSNYAMSWVRQAPGKGLEWVSGINVAY |
| | GITSYADSYKGRFTISRDNTKNTLYLQLNSLKTEDTAIYYCVKHSGTTIPRGFISYTKRG |
| | QGTQVTVSS |
| 24 | QVQLQESGGGSVQAGGSLRLSCVASASTYCTYDMHWYRQAPGKGREFVSAIDSDGTTRYA |
| | DSVKGRFTISQGTAKNTVYLQMNSLQPEDTAMYYCKTVCVVGSRWSDYWGQGTQVTVSS |
| | GGGSQVQLQESGGGLVQPGGSLRLSCAASGFSESSYAMKWVRQAPGKGLEWVSTISSGGS |
| | STNYADSVKGRFTISRDNAKNTLYLQLNSLKIEDTAMYYCAKAIVPTGATMERGQGTQVT |
| | VSS |
| 25 | QVQLQESGGGSVQAGGSLRLSCVASASTYCTYDMHWYRQAPGKGREFVSAIDSDGTTRYA |
| | DSVKGRFTISQGTAKNTVYLQMNSLQPEDTAMYYCKTVCVVGSRWSDYWGQGTQVTVSS |
| | GGGSQVQLQESGGGSVQSGGSLRLSCAASGFTYSTSNSWMAWFRQAPGKEREGVAAIYTV |
| | GGSIFYADSVRGRFTISQDATKNMFYLQMNTLKPEDTAMYYCAAASGRLRGKWFWPYEY |
| | NYWGQGTQVTVSS |
| 26 | QVQLQESGGGSVQAGGSLRLSCVASASTYCTYDMHWYRQAPGKGREFVSAIDSDGTTRYA |
| | DSVKGRFTISQGTAKNTVYLQMNSLQPEDTAMYYCKTVCVVGSRWSDYWGQGTQVTVSS |
| | GGGSQVQLQESGGGSVQAGGSLRLSCVASGYVSCDYFLPSWYRQAPGKEREFVSIIDGTGS |
| | TSYAASVKGRFTASQDKGKNIAYLQMNTLKPEDTAMYYCKASCVRGRAISEYWGQGTQV |
| | TVSS |
| 27 | QVQLQESGGGSVQAGGSLRLSCVASASTYCTYDMHWYRQAPGKGREFVSAIDSDGTTRYA |
| | DSVKGRFTISQGTAKNTVYLQMNSLQPEDTAMYYCKTVCVVGSRWSDYWGQGTQVTVSS |
| | GGGSQVQLQESGGGSVQAGGSLRLSCRASGSTYSNYCLGWFRQITGKEREGVAVINWVGG |
| | MLYFADSVKGRFTVSQDQAKNTVYLQMNSLKPEDTAMYYCAAESVSSFSCGGWLTRPDR |
| | VPYWGQGTQVTVSS |
| 28 | QVQLQESGGGSVQAGGSLRLSCVASASTYCTYDMHWYRQAPGKGREFVSAIDSDGTTRYA |
| | DSVKGRFTISQGTAKNTVYLQMNSLQPEDTAMYYCKTVCVVGSRWSDYWGQGTQVTVSS |
| | GGGSQVQLQESGGGLVQPGGSLRLSCAASGFTFSLSSMSWVRQAPGKGLEWVSAISSGGA |
| | STYYTDSVKGRFTISRDNAKNMLYLQLNSLKTEDTAMYYCAKGGSGYGDASRMTSPGSQG |
| | TQVTVSS |
| 29 | QVQLQESGGGSVQAGGSLTLSCAASEYAYSTCNMGWYRQAPGKERELVSAFISDGSTYYA |
| | DSVKGRFTITRDNAKNTVYLQMNSLKPEDTAIYYCSANCYRRLRNYWGQGTQVTVSSGG |
| | GSQVQLQESGGGLVQPGGSLRLSCAASGFTFSSYPMSWVRQAPGKGLEWISTISAGGDTIL |
| | YADSVKGRFTSSRDNAKNTLYLQLNSLKTEDAAIYYCAKRIDCNSGYCYRRNYWGQGTQV |
| | TVSS |
| 30 | QVQLQESGGGSVQAGGSLTLSCAASEYAYSTCNMGWYRQAPGKERELVSAFISDGSTYYA |
| | DSVKGRFTITRDNAKNTVYLQMNSLKPEDTAIYYCSANCYRRLRNYWGQGTQVTVSSGG |
| | GSQVQLQESGGGLVQPGESLRLSCTASGFTFSNYAMSWVRQAPGKGLEWVSGINVAYGIT |
| | SYADSVKGRFTISRDNTKNTLYLQLNSLKTEDTAIYYCVKHSGTTIPRGFISYTKRGQGT |
| | QVTVSS |
| 31 | QVQLQESGGGSVQAGGSLTLSCAASEYAYSTCNMGWYRQAPGKERELVSAFISDGSTYYA |
| | DSVKGRFTITRDNAKNTVYLQMNSLKPEDTAIYYCSANCYRRLRNYWGQGTQVTVSSGGG |
| | SQVQLQESGGGLVQPGGSLRLSCAASGFSFSSYAMKWVRQAPGKGLEWVSTISSGGSSTN |
| | YADSVKGRFTISRDNAKNTLYLQLNSLKIEDTAMYYCAKAIVPTGATMERGQGTQVTVSS |
| 32 | QVQLQESGGGSVQAGGSLTLSCAASEYAYSTCNMGWYRQAPGKERELVSAFISDGSTYYA |
| | DSVKGRFTITRDNAKNTVYLQMNSLKPEDTAIYYCSANCYRRLRNYWGQGTQVTVSSGGG |
| | SQVQLQESGGGSVQSGGSLRLSCAASGFTYSTSNSWMAWFRQAPGKEREGVAAIYTVGG |
| | SIFYADSVRGRFTISQDATKNMFYLQMNTLKPEDTAMYYCAAASGRLRGKWFWPYEYNY |
| | WGQGTQVTVSS |
| 33 | QVQLQESGGGSVQAGGSLTLSCAASEYAYSTCNMGWYRQAPGKERELVSAFISDGSTYYA |
| | DSVKGRFTITRDNAKNTVYLQMNSLKPEDTAIYYCSANCYRRLRNYWGQGTQVTVSSGGG |
| | SQVQLQESGGGSVQAGGSLRLSCVASGYVSCDYFLPSWYRQAPGKEREFVSIIDGTGSTS |
| | YAASVKGRFTASQDKGENIAYLQMNTLKPEDTAMYYCKASCVRGRAISEYWGQGTQVTV |
| 34 | QVQLQESGGGSVQAGGSLTLSCAASEYAYSTCNMGWYRQAPGKERELVSAFISDGSTYYA |
| | DSVKGRFTITRDNAKNTVYLQMNSLKPEDTAIYYCSANCYRRLRNYWGQGTQVTVSSGG |
| | GSQVQLQESGGGSVQAGGSLRLSCRASGSTYSNYCLGWFRQITGKEREGVAVINWVGGML |
| | YFADSVKGRFTVSQDQAKNTVYLQMNSLKPEDTAMYYCAAESVSSFSCGGWLTRPDRVP |
| | YWGQGTQVTVSS |
| 35 | QVQLQESGGGSVQAGGSLTLSCAASEYAYSTCNMGWYRQAPGKERELVSAFISDGSTYYA |
| | DSVKGRFTITRDNAKNTVYLQMNSLKPEDTAIYYCSANCYRRLRNYWGQGTQVTVSSGG |
| | GSQVQLQESGGGLVQPGGSLRLSCAASGFTFSLSSMSWVRQAPGKGLEWVSAISSGGASTY |
| | YTDSVKGRFTISRDNAKNMLYLQLNSLKTEDTAMYYCAKGGSGYGDASRMTSPGSQGTQ |
| | VTVSS |
| 36 | QVQLQESGGGLVQPGGSLRLSCTASGLTFDDSVMGWFRQAPGKGREAVSCISSSGANAFY |
| | ADSVKGRFTISRDNAKNTLYLQMNSLKPEDTATYYCKRGHACAGYYPIPYDDYWGQGTQ |
| | VTVSSGGGSQVQLQESGGGLVQPGGSLRLSCAASGFTFSSYPMSWVRQAPGKGLEWISTIS |
| | AGGDTTLYADSVKGRFTSSRDNAKNTLYLQLNSLKTEDAAIYYCAKRIDCNSGYCYRRNY |
| | WGQGTQVTVSS |
| 37 | QVQLQESGGGLVQPGGSLRLSCTASGLTFDDSVMGWFRQAPGKGREAVSCISSSGANAFY |
| | ADSVKGRFTISRDNAKNTLYLQMNSLKPEDTATYYCKRGHACAGYYPIPYDDYWGQGTQ |
| | VTVSSGGGSQVQLQESGGGLVQPGESLRLSCTASGFTFSNYAMSWVRQAPGKGLEWVSGI |
| | NVAYGITSYADSVKGRFTISRDNIKNILYLQLNSLKTEDTAIYYCVKHSGTTIPRGFIS |
| | YTKRGQGTQVTVSS |
| 38 | QVQLQESGGGLVQPGGSLRLSCTASGLTFDDSVMGWFRQAPGKGREAVSCISSSGANAFY |
| | ADSVKGRFTISRDNAKNTLYLQMNSLKPEDTATYYCKRGHACAGYYPIPYDDYWGQGTQ |
| | VTVSSGGGSQVQLQESGGGLVQPGGSLRLSCAASGFSFSSYAMKWVRQAPGKGLEWVSTI |
| | SSGGSSTNYADSVKGRFTISRDNAKNTLYLQLNSLKIEDTAMYYCAKAIVPTGATMERGQG |
| | TQVTVSS |
| 39 | QVQLQESGGGLVQPGGSLRLSCTASGLTFDDSVMGWFRQAPGKGREAVSCISSSGANAFY |
| | ADSVKGRFTISRDNAKNTLYLQMNSLKPEDTATYYCKRGHACAGYYPIPYDDYWGQGTQ |
| | VTVSSGGGSQVQLQESGGGSVQSGGSLRLSCAASGFTYSTSNSWMAWFRQAPGKEREGY |
| | AAIYTYGGSIFYADSVRGRFTISQDATKNMFYLQMNTLKPEDTAMYYCAAASGRLRGKWF |
| | WPYEYNYWGQGTQVTVSS |
| 40 | QVQLQESGGGLVQPGGSLRLSCTASGLTFDDSVMGWFRQAPGKGREAVSCISSSGANAFY |
| | ADSVKGRFTISRDNAKNTLYLQMNSLKPEDTATYYCKRGHACAGYYPIPYDDYWGQGTQV |
| | TVSSGGGSQVQLQESGGGSVQAGGSLRLSCVASGYVSCDYFLPSWYRQAPGKEREFVSII |
| | DGTGSTSYAASVKGRFTASQDKGKNIAYLQMNTLKPEDTAMYYCKASCVRGRAISEYWG |
| | QGTQVTVSS |
| 41 | QVQLQESGGGLVQPGGSLRLSCTASGLTFDDSVMGWFRQAPGKGREAVSCISSSGANAFY |
| | ADSYKGRFTISRDNAKNTLYLQMNSLKPEDTATYYCKRGHACAGYYPIPYDDYWGQGTQV |
| | TVSSGGGSQVQLQESGGGSVQAGGSLRLSCRASGSTYSNYCLGWFRQITGKEREGVAVI |
| | NWVGGMLYFADSVKGRFTVSQDQAKNTVYLQMNSLKPEDTAMYYCAAESVSSFSCGGW |
| | LTRPDRVPYWGQGTQVTVSS |
| 42 | QVQLQESGGGLVQPGGSLRLSCTASGLTFDDSVMGWFRQAPGKGREAVSCISSSGANAFY |
| | ADSVKGRFTISRDNAKNTLYLQMNSLKPEDTATYYCKRGHACAGYYPIPYDDYWGQGTQV |
| | TVSSGGGSQVQLQESGGGLVQPGGSLRLSCAASGFTFSLSSMSWVRQAPGKGLEWVSAIS |
| | SGGASTYYTDSVKGRFTISRDNAKNMLYLQLNSLKTEDTAMYYCAKGGSGYGDASRMTSP |
| | GSQGTQVTVSS |

In some embodiments, an IL27R binding protein described herein (e.g., an IL27R binding protein comprising a sequence of any one of SEQ ID NOS: 1-42) is encoded by an isolated nucleic acid that is substantially identical to a sequence of any one of SEQ ID NOS: 109-150, as listed in Table 2C below. In some embodiments, an IL27R binding protein described herein (e.g., an IL27R binding protein comprising a sequence of any one of SEQ ID NOS: 1-42) is encoded by an isolated nucleic acid comprising a sequence having at least 90% (e.g., 91%, 92%, 93%, 94%, 95%, 96%, 97%, 98%, 99%, or 100%) sequence identity to a sequence of any one of SEQ ID NOS: 109-150, as listed in Table 2C below.

TABLE 2C

| SEQ | |
|---|---|
| ID | |
| NO | Sequence |
| 109 | CAGGTGCAGCTGCAGGAGAGCGGCGGCGGCAGCGTGCAGGCCGGGGGCAGCCTGAGG |
| | CTGAGCTGCACCGCCAGCGGCGCCATCGCCAGCGGCTACATCGACAGCAGGTGGTGCA |
| | TGGCCTGGTTCAGGCAGGCCCCCGGCAAGGAGAGGGAGGGCGTGGCCGCCATCTGGCC |
| | CGGCGGGGGCCTGACCGTGTACGCCGACAGCGTGAAGGGCAGGTTCACCATCAGCAGG |
| | GACCACGCCAAGAACACCCTGTACCTGCAGATGAACAACCTGAAGCCCGAGGACACCG |
| | CCATGTACTACTGCGCCGCCGGCAGCCCCAGGATGTGCCCCAGCCTGGAGTTCGGCTTC |
| | GACTACTGGGGCCAGGGCACCCAGGTGACCGTCTCGAGTGGCGGCGGATCCCAGGTGC |
| | AGCTGCAGGAGAGCGGGGGCGGCCTGGTGCAGCCCGGGGGCAGCCTGAGGCTGAGCTG |
| | CGCCGCCAGCGGCTTCACCTTCAGCAGCTACCCCATGAGCTGGGTGAGGCAGGCCCCC |
| | GGCAAGGGCCTGGAGTGGATCAGCACCATCAGCGCCGGCGGCGACACCACCCTGTACG |
| | CCGACAGCGTGAAGGGCAGGTTCACCAGCAGCAGGGACAACGCCAAGAACACCCTGTA |
| | CCTGCAGCTGAACAGCCTGAAGACCGAGGACGCCGCCATCTACTACTGCGCCAAGAGG |
| | ATCGACTGCAACAGCGGCTACTGCTACAGGAGGAACTACTGGGGCCAGGGCACCCAGG |
| | TGACCGTGAGCGCTAGCCACCACCACCACCACCACCACCAC |
| 110 | CAGGTGCAGCTGCAGGAGAGCGGCGGGGCAGCGTGCAGGCCGGCGGCAGCCTGAGG |
| | CTGAGCTGCACCGCCAGCGGCGCCATCGCCAGCGGCTACATCGACAGCAGGTGGTGCA |
| | TGGCCTGGTTCAGGCAGGCCCCCGGCAAGGAGAGGGAGGGCGTGGCCGCCATCTGGCC |
| | CGGCGGCGGCCTGACCGTGTACGCCGACAGCGTGAAGGGCAGGTTCACCATCAGCAGG |
| | GACCACGCCAAGAACACCCTGTACCTGCAGATGAACAACCTGAAGCCCGAGGACACCG |
| | CCATGTACTACTGCGCCGCCGGCAGCCCCAGGATGTGCCCCAGCCTGGAGTTCGGCTTC |
| | GACTACTGGGGCCAGGGCACCCAGGTGACCGTCTCGAGTGGCGGCGGATCCCAGGTGC |
| | AGCTGCAGGAGAGCGGCGGCGGCCTGGTGCAGCCCGGCGAGAGCCTGAGGCTGAGCTG |
| | CACCGCCAGCGGCTTCACCTTCAGCAACTACGCCATGAGCTGGGTGAGGCAGGCCCCC |
| | GGCAAGGGCCTGGAGTGGGTGAGCGGCATCAACGTGGCCTACGGCATCACCAGCTACG |
| | CCGACAGCGTGAAGGGCAGGTTCACCATCAGCAGGGACAACACCAAGAACACCCTGTA |
| | CCTGCAGCTGAACAGCCTGAAGACCGAGGACACCGCCATCTACTACTGCGTGAAGCAC |
| | AGCGGCACCACCATCCCCAGGGGCTTCATCAGCTACACCAAGAGGGGCCAGGGCACCC |
| | AGGTGACCGTGAGCGCTAGCCACCACCACCACCACCACCACCAC |
| 111 | CAGGTGCAGCTGCAGGAGAGCGGCGGCGGCAGCGTGCAGGCCGGGGGCAGCCTGAGG |
| | CTGAGCTGCACCGCCAGCGGCGCCATCGCCAGCGGCTACATCGACAGCAGGTGGTGCA |
| | TGGCCTGGTTCAGGCAGGCCCCCGGCAAGGAGAGGGAGGGCGTGGCCGCCATCTGGCC |
| | CGGCGGGGGCCTGACCGTGTACGCCGACAGCGTGAAGGGCAGGTTCACCATCAGCAGG |
| | GACCACGCCAAGAACACCCTGTACCTGCAGATGAACAACCTGAAGCCCGAGGACACCG |
| | CCATGTACTACTGCGCCGCCGGCAGCCCCAGGATGTGCCCCAGCCTGGAGTTCGGCTTC |
| | GACTACTGGGGCCAGGGCACCCAGGTGACCGTCTCGAGTGGGGGGGGATCCCAGGTGC |
| | AGCTGCAGGAGAGCGGGGGGGGCCTGGTGCAGCCCGGGGGCAGCCTGAGGCTGAGCTG |
| | CGCCGCCAGCGGCTTCAGCTTCAGCAGCTACGCCATGAAGTGGGTGAGGCAGGCCCCC |
| | GGCAAGGGCCTGGAGTGGGTGAGCACCATCAGCAGCGGCGGCAGCAGCACCAACTAC |
| | GCCGACAGCGTGAAGGGCAGGTTCACCATCAGCAGGGACAACGCCAAGAACACCCTGT |
| | ACCTGCAGCTGAACAGCCTGAAGATCGAGGACACCGCCATGTACTACTGCGCCAAGGC |
| | CATCGTGCCCACCGGCGCCACCATGGAGAGGGGCCAGGGCACCCAGGTGACCGTGAGC |
| | GCTAGCCACCACCACCACCACCACCACCAC |
| 112 | CAGGTGCAGCTGCAGGAGAGCGGCGGCGGCAGCGTGCAGGCCGGCGGCAGCCTGAGG |
| | CTGAGCTGCACCGCCAGCGGCGCCATCGCCAGCGGCTACATCGACAGCAGGTGGTGCA |
| | TGGCCTGGTTCAGGCAGGCCCCCGGCAAGGAGAGGGAGGGCGTGGCCGCCATCTGGCC |
| | CGGCGGCGGCCTGACCGTGTACGCCGACAGCGTGAAGGGCAGGTTCACCATCAGCAGG |
| | GACCACGCCAAGAACACCCTGTACCTGCAGATGAACAACCTGAAGCCCGAGGACACCG |
| | CCATGTACTACTGCGCCGCCGGCAGCCCCAGGATGTGCCCCAGCCTGGAGTTCGGCTTC |
| | GACTACTGGGGCCAGGGCACCCAGGTGACCGTCTCGAGTGGCGGCGGATCCCAGGTGC |
| | AGCTGCAGGAGAGCGGCGGCGGCAGCGTGCAGAGCGGCGGCAGCCTGAGGCTGAGCT |
| | GCGCCGCCAGCGGCTTCACCTACAGCACCAGCAACAGCTGGATGGCCTGGTTCAGGCA |
| | GGCCCCCGGCAAGGAGAGGGAGGGCGTGGCCGCCATCTACACCGTGGGGGGCAGCATC |
| | TTCTACGCCGACAGCGTGAGGGGCAGGTTCACCATCAGCCAGGACGCCACCAAGAACA |
| | TGTTCTACCTGCAGATGAACACCCTGAAGCCCGAGGACACCGCCATGTACTACTGCGCC |
| | GCCGCCAGCGGCAGGCTGAGGGGCAAGTGGTTCTGGCCCTACGAGTACAACTACTGGG |
| | GCCAGGGCACCCAGGTGACCGTGAGCGCTAGCCACCACCACCACCACCACCACCAC |
| 113 | CAGGTGCAGCTGCAGGAGAGCGGCGGCGGCAGCGTGCAGGCCGGCGGCAGCCTGAGG |
| | CTGAGCTGCACCGCCAGCGGCGCCATCGCCAGCGGCTACATCGACAGCAGGTGGTGCA |
| | TGGCCTGGTTCAGGCAGGCCCCCGGCAAGGAGAGGGAGGGCGTGGCCGCCATCTGGCC |
| | CGGCGGCGGCCTGACCGTGTACGCCGACAGCGTGAAGGGCAGGTTCACCATCAGCAGG |
| | GACCACGCCAAGAACACCCTGTACCTGCAGATGAACAACCTGAAGCCCGAGGACACCG |
| | CCATGTACTACTGCGCCGCCGGCAGCCCCAGGATGTGCCCCAGCCTGGAGTTCGGCTTC |
| | GACTACTGGGGCCAGGGCACCCAGGTGACCGTCTCGAGTGGGGGGGGATCCCAGGTGC |
| | AGCTGCAGGAGAGCGGCGGCGGCAGCGTGCAGGCCGGCGGCAGCCTGAGGCTGAGCT |
| | GCGTGGCCAGCGGCTACGTGAGCTGCGACTACTTCCTGCCCAGCTGGTACAGGCAGGC |
| | CCCCGGCAAGGAGAGGGAGTTCGTGAGCATCATCGACGGCACCGGCAGCACCAGCTAC |
| | GCCGCCAGCGTGAAGGGCAGGTTCACCGCCAGCCAGGACAAGGGCAAGAACATCGCCT |
| | ACCTGCAGATGAACACCCTGAAGCCCGAGGACACCGCCATGTACTACTGCAAGGCCAG |
| | CTGCGTGAGGGGCAGGGCCATCAGCGAGTACTGGGGCCAGGGCACCCAGGTGACCGTG |
| | AGCGCTAGCCACCACCACCACCACCACCACCAC |
| 114 | CAGGTGCAGCTGCAGGAGAGCGGCGGCGGCAGCGTGCAGGCCGGCGGCAGCCTGAGG |
| | CTGAGCTGCACCGCCAGCGGCGCCATCGCCAGCGGCTACATCGACAGCAGGTGGTGCA |
| | TGGCCTGGTTCAGGCAGGCCCCCGGCAAGGAGAGGGAGGGCGTGGCCGCCATCTGGCC |
| | CGGCGGCGGCCTGACCGTGTACGCCGACAGCGTGAAGGGCAGGTTCACCATCAGCAGG |
| | GACCACGCCAAGAACACCCTGTACCTGCAGATGAACAACCTGAAGCCCGAGGACACCG |
| | CCATGTACTACTGCGCCGCCGGCAGCCCCAGGATGTGCCCCAGCCTGGAGTTCGGCTTC |
| | GACTACTGGGGCCAGGGCACCCAGGTGACCGTCTCGAGTGGCGGCGGATCCCAGGTGC |
| | AGCTGCAGGAGAGCGGCGGCGGCAGCGTGCAGGCCGGCGGCAGCCTGAGGCTGAGCT |
| | GCAGGGCCAGCGGCAGCACCTACAGCAACTACTGCCTGGGCTGGTTCAGGCAGATCAC |
| | CGGCAAGGAGAGGGAGGGCGTGGCCGTGATCAACTGGGTGGGCGGCATGCTGTACTTC |
| | GCCGACAGCGTGAAGGGCAGGTTCACCGTGAGCCAGGACCAGGCCAAGAACACCGTGT |
| | ACCTGCAGATGAACAGCCTGAAGCCCGAGGACACCGCCATGTACTACTGCGCCGCCGA |
| | GAGCGTGAGCAGCTTCAGCTGCGGCGGCTGGCTGACCAGGCCCGACAGGGTGCCCTAC |
| | TGGGGCCAGGGCACCCAGGTGACCGTGAGCGCTAGCCACCACCACCACCACCACCACC |
| | AC |
| 115 | CAGGTGCAGCTGCAGGAGAGCGGCGGCGGCAGCGTGCAGGCCGGGGGCAGCCTGAGG |
| | CTGAGCTGCACCGCCAGCGGCGCCATCGCCAGCGGCTACATCGACAGCAGGTGGTGCA |
| | TGGCCTGGTTCAGGCAGGCCCCCGGCAAGGAGAGGGAGGGCGTGGCCGCCATCTGGCC |
| | CGGCGGCGGCCTGACCGTGTACGCCGACAGCGTGAAGGGCAGGTTCACCATCAGCAGG |
| | GACCACGCCAAGAACACCCTGTACCTGCAGATGAACAACCTGAAGCCCGAGGACACCG |
| | CCATGTACTACTGCGCCGCCGGCAGCCCCAGGATGTGCCCCAGCCTGGAGTTCGGCTTC |
| | GACTACTGGGGCCAGGGCACCCAGGTGACCGTCTCGAGTGGCGGCGGATCCCAGGTGC |
| | AGCTGCAGGAGAGCGGCGGCGGCCTGGTGCAGCCCGGGGGCAGCCTGAGGCTGAGCTG |
| | CGCCGCCAGCGGCTTCACCTTCAGCCTGAGCAGCATGAGCTGGGTGAGGCAGGCCCCC |
| | GGCAAGGGCCTGGAGTGGGTGAGCGCCATCAGCAGCGGCGGCGCCAGCACCTACTACA |
| | CCGACAGCGTGAAGGGCAGGTTCACCATCAGCAGGGACAACGCCAAGAACATGCTGTA |
| | CCTGCAGCTGAACAGCCTGAAGACCGAGGACACCGCCATGTACTACTGCGCCAAGGGC |
| | GGCAGCGGCTACGGCGACGCCAGCAGGATGACCAGCCCCGGCAGCCAGGGCACCCAG |
| | GTGACCGTGAGCGCTAGCCACCACCACCACCACCACCACCAC |
| 116 | CAGGTGCAGCTGCAGGAGAGCGGCGGGGGCAGCGTGCAGGCCGGCGGCAGCCTGAGG |
| | CTGAGCTGCACCGCCCCCGGCTTCACCAGCAACAGCTGCGGCATGGACTGGTACAGGC |
| | AGGCCCCCGGCAAGGAGAGGGAGTTCGTGAGCAGCATCAGCACCGACGGCACCACCG |
| | GCTACGCCGACAGCGTGAAGGGCAGGTTCACCATCAGCAAGGACAAGGCCAAGGACA |
| | CCGTGTACCTGCAGATGAACAGCCTGAAGCCCGAGGACACCGGCATGTACAGCTGCAA |
| | GACCAAGGACGGCACCATCGCCACCATGGAGCTGTGCGACTTCGGCTACTGGGGCCAG |
| | GGCACCCAGGTGACCGTCTCGAGTGGCGGCGGATCCCAGGTGCAGCTGCAGGAGAGCG |
| | GCGGCGGCCTGGTGCAGCCCGGCGGCAGCCTGAGGCTGAGCTGCGCCGCCAGCGGCTT |
| | CACCTTCAGCAGCTACCCCATGAGCTGGGTGAGGCAGGCCCCCGGCAAGGGCCTGGAG |
| | TGGATCAGCACCATCAGCGCCGGCGGCGACACCACCCTGTACGCCGACAGCGTGAAGG |
| | GCAGGTTCACCAGCAGCAGGGACAACGCCAAGAACACCCTGTACCTGCAGCTGAACAG |
| | CCTGAAGACCGAGGACGCCGCCATCTACTACTGCGCCAAGAGGATCGACTGCAACAGC |
| | GGCTACTGCTACAGGAGGAACTACTGGGGCCAGGGCACCCAGGTGACCGTGAGCGCTA |
| | GCCACCACCACCACCACCACCACCAC |
| 117 | CAGGTGCAGCTGCAGGAGAGCGGCGGCGGCAGCGTGCAGGCCGGCGGCAGCCTGAGG |
| | CTGAGCTGCACCGCCCCCGGCTTCACCAGCAACAGCTGCGGCATGGACTGGTACAGGC |
| | AGGCCCCCGGCAAGGAGAGGGAGTTCGTGAGCAGCATCAGCACCGACGGCACCACCG |
| | GCTACGCCGACAGCGTGAAGGGCAGGTTCACCATCAGCAAGGACAAGGCCAAGGACA |
| | CCGTGTACCTGCAGATGAACAGCCTGAAGCCCGAGGACACCGGCATGTACAGCTGCAA |
| | GACCAAGGACGGCACCATCGCCACCATGGAGCTGTGCGACTTCGGCTACTGGGGCCAG |
| | GGCACCCAGGTGACCGTCTCGAGTGGCGGCGGATCCCAGGTGCAGCTGCAGGAGAGCG |
| | GCGGCGGCCTGGTGCAGCCCGGCGAGAGCCTGAGGCTGAGCTGCACCGCCAGCGGCTT |
| | CACCTTCAGCAACTACGCCATGAGCTGGGTGAGGCAGGCCCCCGGCAAGGGCCTGGAG |
| | TGGGTGAGCGGCATCAACGTGGCCTACGGCATCACCAGCTACGCCGACAGCGTGAAGG |
| | GCAGGTTCACCATCAGCAGGGACAACACCAAGAACACCCTGTACCTGCAGCTGAACAG |
| | CCTGAAGACCGAGGACACCGCCATCTACTACTGCGTGAAGCACAGCGGCACCACCATC |
| | CCCAGGGGCTTCATCAGCTACACCAAGAGGGGCCAGGGCACCCAGGTGACCGTGAGCG |
| | CTAGCCACCACCACCACCACCACCACCAC |
| 118 | CAGGTGCAGCTGCAGGAGAGCGGCGGCGGCAGCGTGCAGGCCGGCGGCAGCCTGAGG |
| | CTGAGCTGCACCGCCCCCGGCTTCACCAGCAACAGCTGCGGCATGGACTGGTACAGGC |
| | AGGCCCCCGGCAAGGAGAGGGAGTTCGTGAGCAGCATCAGCACCGACGGCACCACCG |
| | GCTACGCCGACAGCGTGAAGGGCAGGTTCACCATCAGCAAGGACAAGGCCAAGGACA |
| | CCGTGTACCTGCAGATGAACAGCCTGAAGCCCGAGGACACCGGCATGTACAGCTGCAA |
| | GACCAAGGACGGCACCATCGCCACCATGGAGCTGTGCGACTTCGGCTACTGGGGCCAG |
| | GGCACCCAGGTGACCGTCTCGAGTGGCGGCGGATCCCAGGTGCAGCTGCAGGAGAGCG |
| | GCGGCGGCCTGGTGCAGCCCGGCGGCAGCCTGAGGCTGAGCTGCGCCGCCAGCGGCTT |
| | CAGCTTCAGCAGCTACGCCATGAAGTGGGTGAGGCAGGCCCCCGGCAAGGGCCTGGAG |
| | TGGGTGAGCACCATCAGCAGCGGGGGCAGCAGCACCAACTACGCCGACAGCGTGAAGG |
| | GCAGGTTCACCATCAGCAGGGACAACGCCAAGAACACCCTGTACCTGCAGCTGAACAG |
| | CCTGAAGATCGAGGACACCGCCATGTACTACTGCGCCAAGGCCATCGTGCCCACCGGC |
| | GCCACCATGGAGAGGGGCCAGGGCACCCAGGTGACCGTGAGCGCTAGCCACCACCACC |
| | ACCACCACCACCAC |
| 119 | CAGGTGCAGCTGCAGGAGAGCGGCGGGGGCAGCGTGCAGGCCGGCGGCAGCCTGAGG |
| | CTGAGCTGCACCGCCCCCGGCTTCACCAGCAACAGCTGCGGCATGGACTGGTACAGGC |
| | AGGCCCCCGGCAAGGAGAGGGAGTTCGTGAGCAGCATCAGCACCGACGGCACCACCG |
| | GCTACGCCGACAGCGTGAAGGGCAGGTTCACCATCAGCAAGGACAAGGCCAAGGACA |
| | CCGTGTACCTGCAGATGAACAGCCTGAAGCCCGAGGACACCGGCATGTACAGCTGCAA |
| | GACCAAGGACGGCACCATCGCCACCATGGAGCTGTGCGACTTCGGCTACTGGGGCCAG |
| | GGCACCCAGGTGACCGTCTCGAGTGGGGGGGGATCCCAGGTGCAGCTGCAGGAGAGCG |
| | GCGGCGGCAGCGTGCAGAGCGGCGGCAGCCTGAGGCTGAGCTGCGCCGCCAGCGGCTT |
| | CACCTACAGCACCAGCAACAGCTGGATGGCCTGGTTCAGGCAGGCCCCCGGCAAGGAG |
| | AGGGAGGGCGTGGCCGCCATCTACACCGTGGGGGGCAGCATCTTCTACGCCGACAGCG |
| | TGAGGGGCAGGTTCACCATCAGCCAGGACGCCACCAAGAACATGTTCTACCTGCAGAT |
| | GAACACCCTGAAGCCCGAGGACACCGCCATGTACTACTGCGCCGCCGCCAGCGGCAGG |
| | CTGAGGGGCAAGTGGTTCTGGCCCTACGAGTACAACTACTGGGGCCAGGGCACCCAGG |
| | TGACCGTGAGCGCTAGCCACCACCACCACCACCACCACCAC |
| 120 | CAGGTGCAGCTGCAGGAGAGCGGCGGCGGCAGCGTGCAGGCCGGGGGCAGCCTGAGG |
| | CTGAGCTGCACCGCCCCCGGCTTCACCAGCAACAGCTGCGGCATGGACTGGTACAGGC |
| | AGGCCCCCGGCAAGGAGAGGGAGTTCGTGAGCAGCATCAGCACCGACGGCACCACCG |
| | GCTACGCCGACAGCGTGAAGGGCAGGTTCACCATCAGCAAGGACAAGGCCAAGGACA |
| | CCGTGTACCTGCAGATGAACAGCCTGAAGCCCGAGGACACCGGCATGTACAGCTGCAA |
| | GACCAAGGACGGCACCATCGCCACCATGGAGCTGTGCGACTTCGGCTACTGGGGCCAG |
| | GGCACCCAGGTGACCGTCTCGAGTGGCGGCGGATCCCAGGTGCAGCTGCAGGAGAGCG |
| | GCGGCGGCAGCGTGCAGGCCGGCGGCAGCCTGAGGCTGAGCTGCGTGGCCAGCGGCTA |
| | CGTGAGCTGCGACTACTTCCTGCCCAGCTGGTACAGGCAGGCCCCCGGCAAGGAGAGG |
| | GAGTTCGTGAGCATCATCGACGGCACCGGCAGCACCAGCTACGCCGCCAGCGTGAAGG |
| | GCAGGTTCACCGCCAGCCAGGACAAGGGCAAGAACATCGCCTACCTGCAGATGAACAC |
| | CCTGAAGCCCGAGGACACCGCCATGTACTACTGCAAGGCCAGCTGCGTGAGGGGCAGG |
| | GCCATCAGCGAGTACTGGGGCCAGGGCACCCAGGTGACCGTGAGCGCTAGCCACCACC |
| | ACCACCACCACCACCAC |
| 121 | CAGGTGCAGCTGCAGGAGAGCGGCGGGGGCAGCGTGCAGGCCGGCGGCAGCCTGAGG |
| | CTGAGCTGCACCGCCCCCGGCTTCACCAGCAACAGCTGCGGCATGGACTGGTACAGGC |
| | AGGCCCCCGGCAAGGAGAGGGAGTTCGTGAGCAGCATCAGCACCGACGGCACCACCG |
| | GCTACGCCGACAGCGTGAAGGGCAGGTTCACCATCAGCAAGGACAAGGCCAAGGACA |
| | CCGTGTACCTGCAGATGAACAGCCTGAAGCCCGAGGACACCGGCATGTACAGCTGCAA |
| | GACCAAGGACGGCACCATCGCCACCATGGAGCTGTGCGACTTCGGCTACTGGGGCCAG |
| | GGCACCCAGGTGACCGTCTCGAGTGGCGGCGGATCCCAGGTGCAGCTGCAGGAGAGCG |
| | GCGGCGGCAGCGTGCAGGCCGGCGGCAGCCTGAGGCTGAGCTGCAGGGCCAGCGGCA |
| | GCACCTACAGCAACTACTGCCTGGGCTGGTTCAGGCAGATCACCGGCAAGGAGAGGGA |
| | GGGCGTGGCCGTGATCAACTGGGTGGGCGGCATGCTGTACTTCGCCGACAGCGTGAAG |
| | GGCAGGTTCACCGTGAGCCAGGACCAGGCCAAGAACACCGTGTACCTGCAGATGAACA |
| | GCCTGAAGCCCGAGGACACCGCCATGTACTACTGCGCCGCCGAGAGCGTGAGCAGCTT |
| | CAGCTGCGGCGGCTGGCTGACCAGGCCCGACAGGGTGCCCTACTGGGGCCAGGGCACC |
| | CAGGTGACCGTGAGCGCTAGCCACCACCACCACCACCACCACCAC |
| 122 | CAGGTGCAGCTGCAGGAGAGCGGCGGCGGCAGCGTGCAGGCCGGGGGCAGCCTGAGG |
| | CTGAGCTGCACCGCCCCCGGCTTCACCAGCAACAGCTGCGGCATGGACTGGTACAGGC |
| | AGGCCCCCGGCAAGGAGAGGGAGTTCGTGAGCAGCATCAGCACCGACGGCACCACCG |
| | GCTACGCCGACAGCGTGAAGGGCAGGTTCACCATCAGCAAGGACAAGGCCAAGGACA |
| | CCGTGTACCTGCAGATGAACAGCCTGAAGCCCGAGGACACCGGCATGTACAGCTGCAA |
| | GACCAAGGACGGCACCATCGCCACCATGGAGCTGTGCGACTTCGGCTACTGGGGCCAG |
| | GGCACCCAGGTGACCGTCTCGAGTGGCGGCGGATCCCAGGTGCAGCTGCAGGAGAGCG |
| | GCGGCGGCCTGGTGCAGCCCGGCGGCAGCCTGAGGCTGAGCTGCGCCGCCAGCGGCTT |
| | CACCTTCAGCCTGAGCAGCATGAGCTGGGTGAGGCAGGCCCCCGGCAAGGGCCTGGAG |
| | TGGGTGAGCGCCATCAGCAGCGGCGGCGCCAGCACCTACTACACCGACAGCGTGAAGG |
| | GCAGGTTCACCATCAGCAGGGACAACGCCAAGAACATGCTGTACCTGCAGCTGAACAG |
| | CCTGAAGACCGAGGACACCGCCATGTACTACTGCGCCAAGGGCGGCAGCGGCTACGGC |
| | GACGCCAGCAGGATGACCAGCCCCGGCAGCCAGGGCACCCAGGTGACCGTGAGCGCTA |
| | GCCACCACCACCACCACCACCACCAC |
| 123 | CAGGTGCAGCTGCAGGAGAGCGGCGGGGGCAGCGTGCAGGCCGGCGGCAGCCTGAGG |
| | CTGAGCTGCGCCGCCAGCGGCTACCCCTACAGCAACGGCTACATGGGCTGGTTCAGGC |
| | AGGCCCCCGGCAAGGAGAGGGAGGGCGTGGCCACCATCTACACCGGCGACGGCAGGA |
| | CCTACTACGCCGACAGCGTGAAGGGCAGGTTCACCATCAGCAGGGACAACGCCAAGAA |
| | CACCGTGGACCTGCAGATGAGCAGCCTGAAGCCCGAGGACACCGCCATGTACTACTGC |
| | GCCGCCAGGGCCGCCCCCCTGTACAGCAGCGGCAGCCCCCTGACCAGGGCCAGGTACA |
| | ACGTGTGGGGCCAGGGCACCCAGGTGACCGTCTCGAGTGGCGGCGGATCCCAGGTGCA |
| | GCTGCAGGAGAGCGGGGGGGGCCTGGTGCAGCCCGGGGGCAGCCTGAGGCTGAGCTGC |
| | GCCGCCAGGGGCTTCACCTTCAGCAGCTACCCCATGAGCTGGGTGAGGCAGGCCCCCG |
| | GCAAGGGCCTGGAGTGGATCAGCACCATCAGCGCCGGCGGCGACACCACCCTGTACGC |
| | CGACAGCGTGAAGGGCAGGTTCACCAGCAGCAGGGACAACGCCAAGAACACCCTGTAC |
| | CTGCAGCTGAACAGCCTGAAGACCGAGGACGCCGCCATCTACTACTGCGCCAAGAGGA |
| | TCGACTGCAACAGGGGCTACTGCTACAGGAGGAACTACTGGGGCCAGGGCACCCAGGT |
| | GACCGTGAGCGCTAGCCACCACCACCACCACCACCACCAC |
| 124 | CAGGTGCAGCTGCAGGAGAGCGGCGGCGGCAGCGTGCAGGCCGGGGGCAGCCTGAGG |
| | CTGAGCTGCGCCGCCAGCGGCTACCCCTACAGCAACGGCTACATGGGCTGGTTCAGGC |
| | AGGCCCCCGGCAAGGAGAGGGAGGGCGTGGCCACCATCTACACCGGCGACGGCAGGA |
| | CCTACTACGCCGACAGCGTGAAGGGCAGGTTCACCATCAGCAGGGACAACGCCAAGAA |
| | CACCGTGGACCTGCAGATGAGCAGCCTGAAGCCCGAGGACACCGCCATGTACTACTGC |
| | GCCGCCAGGGCCGCCCCCCTGTACAGCAGCGGCAGCCCCCTGACCAGGGCCAGGTACA |
| | ACGTGTGGGGCCAGGGCACCCAGGTGACCGTCTCGAGTGGGGGGATCCCAGGTGCA |
| | GCTGCAGGAGAGCGGCGGCGGCCTGGTGCAGCCCGGCGAGAGCCTGAGGCTGAGCTGC |
| | ACCGCCAGCGGCTTCACCTTCAGCAACTACGCCATGAGCTGGGTGAGGCAGGCCCCCG |
| | GCAAGGGCCTGGAGTGGGTGAGCGGCATCAACGTGGCCTACGGCATCACCAGCTACGC |
| | CGACAGCGTGAAGGGCAGGTTCACCATCAGCAGGGACAACACCAAGAACACCCTGTAC |
| | CTGCAGCTGAACAGCCTGAAGACCGAGGACACCGCCATCTACTACTGCGTGAAGCACA |
| | GCGGCACCACCATCCCCAGGGGCTTCATCAGCTACACCAAGAGGGGCCAGGGCACCCA |
| | GGTGACCGTGAGCGCTAGCCACCACCACCACCACCACCACCAC |
| 125 | CAGGTGCAGCTGCAGGAGAGCGGCGGGGGCAGCGTGCAGGCCGGCGGCAGCCTGAGG |
| | CTGAGCTGCGCCGCCAGCGGCTACCCCTACAGCAACGGCTACATGGGCTGGTTCAGGC |
| | AGGCCCCCGGCAAGGAGAGGGAGGGCGTGGCCACCATCTACACCGGCGACGGCAGGA |
| | CCTACTACGCCGACAGCGTGAAGGGCAGGTTCACCATCAGCAGGGACAACGCCAAGAA |
| | CACCGTGGACCTGCAGATGAGCAGCCTGAAGCCCGAGGACACCGCCATGTACTACTGC |
| | GCCGCCAGGGCCGCCCCCCTGTACAGCAGCGGCAGCCCCCTGACCAGGGCCAGGTACA |
| | ACGTGTGGGGCCAGGGCACCCAGGTGACCGTCTCGAGTGGCGGCGGATCCCAGGTGCA |
| | GCTGCAGGAGAGCGGGGGCGGCCTGGTGCAGCCCGGGGGCAGCCTGAGGCTGAGCTGC |
| | GCCGCCAGCGGCTTCAGCTTCAGCAGCTACGCCATGAAGTGGGTGAGGCAGGCCCCCG |
| | GCAAGGGCCTGGAGTGGGTGAGCACCATCAGCAGCGGCGGCAGCAGCACCAACTACGC |
| | CGACAGCGTGAAGGGCAGGTTCACCATCAGCAGGGACAACGCCAAGAACACCCTGTAC |
| | CTGCAGCTGAACAGCCTGAAGATCGAGGACACCGCCATGTACTACTGCGCCAAGGCCA |
| | TCGTGCCCACCGGCGCCACCATGGAGAGGGGCCAGGGCACCCAGGTGACCGTGAGCGC |
| | TAGCCACCACCACCACCACCACCACCAC |
| 126 | CAGGTGCAGCTGCAGGAGAGCGGCGGCGGCAGCGTGCAGGCCGGGGGCAGCCTGAGG |
| | CTGAGCTGCGCCGCCAGCGGCTACCCCTACAGCAACGGCTACATGGGCTGGTTCAGGC |
| | AGGCCCCCGGCAAGGAGAGGGAGGGCGTGGCCACCATCTACACCGGCGACGGCAGGA |
| | CCTACTACGCCGACAGCGTGAAGGGCAGGTTCACCATCAGCAGGGACAACGCCAAGAA |
| | CACCGTGGACCTGCAGATGAGCAGCCTGAAGCCCGAGGACACCGCCATGTACTACTGC |
| | GCCGCCAGGGCCGCCCCCCTGTACAGCAGCGGCAGCCCCCTGACCAGGGCCAGGTACA |
| | ACGTGTGGGGCCAGGGCACCCAGGTGACCGTCTCGAGTGGCGGCGGATCCCAGGTGCA |
| | GCTGCAGGAGAGCGGCGGCGGCAGCGTGCAGAGCGGCGGCAGCCTGAGGCTGAGCTG |
| | CGCCGCCAGCGGCTTCACCTACAGCACCAGCAACAGCTGGATGGCCTGGTTCAGGCAG |
| | GCCCCCGGCAAGGAGAGGGAGGGCGTGGCCGCCATCTACACCGTGGGGGGCAGCATCT |
| | TCTACGCCGACAGCGTGAGGGGCAGGTTCACCATCAGCCAGGACGCCACCAAGAACAT |
| | GTTCTACCTGCAGATGAACACCCTGAAGCCCGAGGACACCGCCATGTACTACTGGGGGG |
| | CCGCCAGCGGCAGGCTGAGGGGCAAGTGGTTCTGGCCCTACGAGTACAACTACTGGGG |
| | CCAGGGCACCCAGGTGACCGTGAGCGCTAGCCACCACCACCACCACCACCACCAC |
| 127 | CAGGTGCAGCTGCAGGAGAGCGGCGGGGGCAGCGTGCAGGCCGGCGGCAGCCTGAGG |
| | CTGAGCTGCGCCGCCAGCGGCTACCCCTACAGCAACGGCTACATGGGCTGGTTCAGGC |
| | AGGCCCCCGGCAAGGAGAGGGAGGGCGTGGCCACCATCTACACCGGCGACGGCAGGA |
| | CCTACTACGCCGACAGCGTGAAGGGCAGGTTCACCATCAGCAGGGACAACGCCAAGAA |
| | CACCGTGGACCTGCAGATGAGCAGCCTGAAGCCCGAGGACACCGCCATGTACTACTGC |
| | GCCGCCAGGGCCGCCCCCCTGTACAGCAGCGGCAGCCCCCTGACCAGGGCCAGGTACA |
| | ACGTGTGGGGCCAGGGCACCCAGGTGACCGTCTCGAGTGGGGGGGGATCCCAGGTGCA |
| | GCTGCAGGAGAGCGGGGGGGGCAGCGTGCAGGCCGGGGGCAGCCTGAGGCTGAGCTG |
| | CGTGGCCAGCGGCTACGTGAGCTGCGACTACTTCCTGCCCAGCTGGTACAGGCAGGCCC |
| | CCGGCAAGGAGAGGGAGTTCGTGAGCATCATCGACGGCACCGGCAGCACCAGCTACGC |
| | CGCCAGCGTGAAGGGCAGGTTCACCGCCAGCCAGGACAAGGGCAAGAACATCGCCTAC |
| | CTGCAGATGAACACCCTGAAGCCCGAGGACACCGCCATGTACTACTGCAAGGCCAGCT |
| | GCGTGAGGGGCAGGGCCATCAGCGAGTACTGGGGCCAGGGCACCCAGGTGACCGTGA |
| | GCGCTAGCCACCACCACCACCACCACCACCAC |
| 128 | CAGGTGCAGCTGCAGGAGAGCGGCGGCGGCAGCGTGCAGGCCGGCGGCAGCCTGAGG |
| | CTGAGCTGCGCCGCCAGCGGCTACCCCTACAGCAACGGCTACATGGGCTGGTTCAGGC |
| | AGGCCCCCGGCAAGGAGAGGGAGGGCGTGGCCACCATCTACACCGGCGACGGCAGGA |
| | CCTACTACGCCGACAGCGTGAAGGGCAGGTTCACCATCAGCAGGGACAACGCCAAGAA |
| | CACCGTGGACCTGCAGATGAGCAGCCTGAAGCCCGAGGACACCGCCATGTACTACTGC |
| | GCCGCCAGGGCCGCCCCCCTGTACAGCAGCGGCAGCCCCCTGACCAGGGCCAGGTACA |
| | ACGTGTGGGGCCAGGGCACCCAGGTGACCGTCTCGAGTGGCGGCGGATCCCAGGTGCA |
| | GCTGCAGGAGAGCGGCGGCGGCAGCGTGCAGGCCGGCGGCAGCCTGAGGCTGAGCTG |
| | CAGGGCCAGCGGCAGCACCTACAGCAACTACTGCCTGGGCTGGTTCAGGCAGATCACC |
| | GGCAAGGAGAGGGAGGGCGTGGCCGTGATCAACTGGGTGGGGGCATGCTGTACTTCG |
| | CCGACAGCGTGAAGGGCAGGTTCACCGTGAGCCAGGACCAGGCCAAGAACACCGTGTA |
| | CCTGCAGATGAACAGCCTGAAGCCCGAGGACACCGCCATGTACTACTGCGCCGCCGAG |
| | AGCGTGAGCAGCTTCAGCTGCGGCGGCTGGCTGACCAGGCCCGACAGGGTGCCCTACT |
| | GGGGCCAGGGCACCCAGGTGACCGTGAGCGCTAGCCACCACCACCACCACCACCACCA |
| | C |
| 129 | CAGGTGCAGCTGCAGGAGAGGGGGGGGCAGCGTGCAGGCCGGCGGCAGCCTGAGG |
| | CTGAGCTGCGCCGCCAGCGGCTACCCCTACAGCAACGGCTACATGGGCTGGTTCAGGC |
| | AGGCCCCCGGCAAGGAGAGGGAGGGCGTGGCCACCATCTACACCGGCGACGGCAGGA |
| | CCTACTACGCCGACAGCGTGAAGGGCAGGTTCACCATCAGCAGGGACAACGCCAAGAA |
| | CACCGTGGACCTGCAGATGAGCAGCCTGAAGCCCGAGGACACCGCCATGTACTACTGC |
| | GCCGCCAGGGCCGCCCCCCTGTACAGCAGCGGCAGCCCCCTGACCAGGGCCAGGTACA |
| | ACGTGTGGGGCCAGGGCACCCAGGTGACCGTCTCGAGTGGCGGCGGATCCCAGGTGCA |
| | GCTGCAGGAGAGCGGCGGCGGCCTGGTGCAGCCCGGCGGCAGCCTGAGGCTGAGCTGC |
| | GCCGCCAGCGGCTTCACCTTCAGCCTGAGCAGCATGAGCTGGGTGAGGCAGGCCCCCG |
| | GCAAGGGCCTGGAGTGGGTGAGCGCCATCAGCAGCGGCGGCGCCAGCACCTACTACAC |
| | CGACAGCGTGAAGGGCAGGTTCACCATCAGCAGGGACAACGCCAAGAACATGCTGTAC |
| | CTGCAGCTGAACAGCCTGAAGACCGAGGACACCGCCATGTACTACTGCGCCAAGGGCG |
| | GCAGCGGCTACGGCGACGCCAGCAGGATGACCAGCCCCGGCAGCCAGGGCACCCAGGT |
| | GACCGTGAGCGCTAGCCACCACCACCACCACCACCACCAC |
| 130 | CAGGTGCAGCTGCAGGAGAGCGGCGGCGGCAGCGTGCAGGCCGGCGGCAGCCTGAGG |
| | CTGAGCTGCGTGGCCAGCGCCAGCACCTACTGCACCTACGACATGCACTGGTACAGGC |
| | AGGCCCCCGGCAAGGGCAGGGAGTTCGTGAGCGCCATCGACAGCGACGGCACCACCAG |
| | GTACGCCGACAGCGTGAAGGGCAGGTTCACCATCAGCCAGGGCACCGCCAAGAACACC |
| | GTGTACCTGCAGATGAACAGCCTGCAGCCCGAGGACACCGCCATGTACTACTGCAAGA |
| | CCGTGTGCGTGGTGGGCAGCAGGTGGAGCGACTACTGGGGCCAGGGCACCCAGGTGAC |
| | CGTCTCGAGTGGCGGCGGATCCCAGGTGCAGCTGCAGGAGAGCGGCGGCGGCCTGGTG |
| | CAGCCCGGCGGCAGCCTGAGGCTGAGCTGCGCCGCCAGCGGCTTCACCTTCAGCAGCT |
| | ACCCCATGAGCTGGGTGAGGCAGGCCCCCGGCAAGGGCCTGGAGTGGATCAGCACCAT |
| | CAGCGCCGGCGGCGACACCACCCTGTACGCCGACAGCGTGAAGGGCAGGTTCACCAGC |
| | AGCAGGGACAACGCCAAGAACACCCTGTACCTGCAGCTGAACAGCCTGAAGACCGAGG |
| | ACGCCGCCATCTACTACTGCGCCAAGAGGATCGACTGCAACAGCGGCTACTGCTACAG |
| | GAGGAACTACTGGGGCCAGGGCACCCAGGTGACCGTGAGCGCTAGCCACCACCACCAC |
| | CACCACCACCAC |
| 131 | CAGGTGCAGCTGCAGGAGAGCGGCGGCGGCAGCGTGCAGGCCGGCGGCAGCCTGAGG |
| | CTGAGCTGCGTGGCCAGCGCCAGCACCTACTGCACCTACGACATGCACTGGTACAGGC |
| | AGGCCCCCGGCAAGGGCAGGGAGTTCGTGAGCGCCATCGACAGCGACGGCACCACCAG |
| | GTACGCCGACAGCGTGAAGGGCAGGTTCACCATCAGCCAGGGCACCGCCAAGAACACC |
| | GTGTACCTGCAGATGAACAGCCTGCAGCCCGAGGACACCGCCATGTACTACTGCAAGA |
| | CCGTGTGCGTGGTGGGCAGCAGGTGGAGCGACTACTGGGGCCAGGGCACCCAGGTGAC |
| | CGTCTCGAGTGGCGGCGGATCCCAGGTGCAGCTGCAGGAGAGCGGCGGCGGCCTGGTG |
| | CAGCCCGGCGAGAGCCTGAGGCTGAGCTGCACCGCCAGCGGCTTCACCTTCAGCAACT |
| | ACGCCATGAGCTGGGTGAGGCAGGCCCCCGGCAAGGGCCTGGAGTGGGTGAGCGGCAT |
| | CAACGTGGCCTACGGCATCACCAGCTACGCCGACAGCGTGAAGGGCAGGTTCACCATC |
| | AGCAGGGACAACACCAAGAACACCCTGTACCTGCAGCTGAACAGCCTGAAGACCGAGG |
| | ACACCGCCATCTACTACTGCGTGAAGCACAGCGGCACCACCATCCCCAGGGGCTTCATC |
| | AGCTACACCAAGAGGGGCCAGGGCACCCAGGTGACCGTGAGCGCTAGCCACCACCACC |
| | ACCACCACCACCAC |
| 132 | CAGGTGCAGCTGCAGGAGAGCGGCGGCGGCAGCGTGCAGGCCGGCGGCAGCCTGAGG |
| | CTGAGCTGCGTGGCCAGCGCCAGCACCTACTGCACCTACGACATGCACTGGTACAGGC |
| | AGGCCCCCGGCAAGGGCAGGGAGTTCGTGAGCGCCATCGACAGCGACGGCACCACCAG |
| | GTACGCCGACAGCGTGAAGGGCAGGTTCACCATCAGCCAGGGCACCGCCAAGAACACC |
| | GTGTACCTGCAGATGAACAGCCTGCAGCCCGAGGACACCGCCATGTACTACTGCAAGA |
| | CCGTGTGCGTGGTGGGCAGCAGGTGGAGCGACTACTGGGGCCAGGGCACCCAGGTGAC |
| | CGTCTCGAGTGGCGGCGGATCCCAGGTGCAGCTGCAGGAGAGCGGCGGCGGCCTGGTG |
| | CAGCCCGGCGGCAGCCTGAGGCTGAGCTGCGCCGCCAGCGGCTTCAGCTTCAGCAGCT |
| | ACGCCATGAAGTGGGTGAGGCAGGCCCCCGGCAAGGGCCTGGAGTGGGTGAGCACCAT |
| | CAGCAGCGGCGGCAGCAGCACCAACTACGCCGACAGCGTGAAGGGCAGGTTCACCATC |
| | AGCAGGGACAACGCCAAGAACACCCTGTACCTGCAGCTGAACAGCCTGAAGATCGAGG |
| | ACACCGCCATGTACTACTGCGCCAAGGCCATCGTGCCCACCGGCGCCACCATGGAGAG |
| | GGGCCAGGGCACCCAGGTGACCGTGAGCGCTAGCCACCACCACCACCACCACCACCAC |
| 133 | CAGGTGCAGCTGCAGGAGAGCGGGGGGGGCAGCGTGCAGGCCGGCGGCAGCCTGAGG |
| | CTGAGCTGCGTGGCCAGCGCCAGCACCTACTGCACCTACGACATGCACTGGTACAGGC |
| | AGGCCCCCGGCAAGGGCAGGGAGTTCGTGAGCGCCATCGACAGCGACGGCACCACCAG |
| | GTACGCCGACAGCGTGAAGGGCAGGTTCACCATCAGCCAGGGCACCGCCAAGAACACC |
| | GTGTACCTGCAGATGAACAGCCTGCAGCCCGAGGACACCGCCATGTACTACTGCAAGA |
| | CCGTGTGCGTGGTGGGCAGCAGGTGGAGCGACTACTGGGGCCAGGGCACCCAGGTGAC |
| | CGTCTCGAGTGGCGGCGGATCCCAGGTGCAGCTGCAGGAGAGCGGCGGCGGCAGCGTG |
| | CAGAGCGGCGGCAGCCTGAGGCTGAGCTGCGCCGCCAGCGGCTTCACCTACAGCACCA |
| | GCAACAGCTGGATGGCCTGGTTCAGGCAGGCCCCCGGCAAGGAGAGGGAGGGCGTGG |
| | CCGCCATCTACACCGTGGGGGCAGCATCTTCTACGCCGACAGCGTGAGGGGCAGGTT |
| | CACCATCAGCCAGGACGCCACCAAGAACATGTTCTACCTGCAGATGAACACCCTGAAG |
| | CCCGAGGACACCGCCATGTACTACTGCGCCGCCGCCAGCGGCAGGCTGAGGGGCAAGT |
| | GGTTCTGGCCCTACGAGTACAACTACTGGGGCCAGGGCACCCAGGTGACCGTGAGCGC |
| | TAGCCACCACCACCACCACCACCACCAC |
| 134 | CAGGTGCAGCTGCAGGAGAGCGGCGGCGGCAGCGTGCAGGCCGGCGGCAGCCTGAGG |
| | CTGAGCTGCGTGGCCAGCGCCAGCACCTACTGCACCTACGACATGCACTGGTACAGGC |
| | AGGCCCCCGGCAAGGGCAGGGAGTTCGTGAGCGCCATCGACAGCGACGGCACCACCAG |
| | GTACGCCGACAGCGTGAAGGGCAGGTTCACCATCAGCCAGGGCACCGCCAAGAACACC |
| | GTGTACCTGCAGATGAACAGCCTGCAGCCCGAGGACACCGCCATGTACTACTGCAAGA |
| | CCGTGTGCGTGGTGGGCAGCAGGTGGAGCGACTACTGGGGCCAGGGCACCCAGGTGAC |
| | CGTCTCGAGTGGCGGCGGATCCCAGGTGCAGCTGCAGGAGAGCGGGGGCGGCAGCGTG |
| | CAGGCCGGCGGCAGCCTGAGGCTGAGCTGCGTGGCCAGCGGCTACGTGAGCTGCGACT |
| | ACTTCCTGCCCAGCTGGTACAGGCAGGCCCCCGGCAAGGAGAGGGAGTTCGTGAGCAT |
| | CATCGACGGCACCGGCAGCACCAGCTACGCCGCCAGCGTGAAGGGCAGGTTCACCGCC |
| | AGCCAGGACAAGGGCAAGAACATCGCCTACCTGCAGATGAACACCCTGAAGCCCGAGG |
| | ACACCGCCATGTACTACTGCAAGGCCAGCTGCGTGAGGGGCAGGGCCATCAGCGAGTA |
| | CTGGGGCCAGGGCACCCAGGTGACCGTGAGCGCTAGCCACCACCACCACCACCACCAC |
| | CAC |
| 135 | CAGGTGCAGCTGCAGGAGAGCGGCGGCGGCAGCGTGCAGGCCGGCGGCAGCCTGAGG |
| | CTGAGCTGCGTGGCCAGCGCCAGCACCTACTGCACCTACGACATGCACTGGTACAGGC |
| | AGGCCCCCGGCAAGGGCAGGGAGTTCGTGAGCGCCATCGACAGCGACGGCACCACCAG |
| | GTACGCCGACAGCGTGAAGGGCAGGTTCACCATCAGCCAGGGCACCGCCAAGAACACC |
| | GTGTACCTGCAGATGAACAGCCTGCAGCCCGAGGACACCGCCATGTACTACTGCAAGA |
| | CCGTGTGCGTGGTGGGCAGCAGGTGGAGCGACTACTGGGGCCAGGGCACCCAGGTGAC |
| | CGTCTCGAGTGGCGGCGGATCCCAGGTGCAGCTGCAGGAGAGCGGCGGCGGCAGCGTG |
| | CAGGCCGGGGCAGCCTGAGGCTGAGCTGCAGGGCCAGCGGCAGCACCTACAGCAACT |
| | ACTGCCTGGGCTGGTTCAGGCAGATCACCGGCAAGGAGAGGGAGGGCGTGGCCGTGAT |
| | CAACTGGGTGGGCGGCATGCTGTACTTCGCCGACAGCGTGAAGGGCAGGTTCACCGTG |
| | AGCCAGGACCAGGCCAAGAACACCGTGTACCTGCAGATGAACAGCCTGAAGCCCGAGG |
| | ACACCGCCATGTACTACTGCGCCGCCGAGAGCGTGAGCAGCTTCAGCTGCGGCGGCTG |
| | GCTGACCAGGCCCGACAGGGTGCCCTACTGGGGCCAGGGCACCCAGGTGACCGTGAGC |
| | GCTAGCCACCACCACCACCACCACCACCAC |
| 136 | CAGGTGCAGCTGCAGGAGAGCGGCGGCGGCAGCGTGCAGGCCGGCGGCAGCCTGAGG |
| | CTGAGCTGCGTGGCCAGCGCCAGCACCTACTGCACCTACGACATGCACTGGTACAGGC |
| | AGGCCCCCGGCAAGGGCAGGGAGTTCGTGAGCGCCATCGACAGCGACGGCACCACCAG |
| | GTACGCCGACAGCGTGAAGGGCAGGTTCACCATCAGCCAGGGCACCGCCAAGAACACC |
| | GTGTACCTGCAGATGAACAGCCTGCAGCCCGAGGACACCGCCATGTACTACTGCAAGA |
| | CCGTGTGCGTGGTGGGCAGCAGGTGGAGCGACTACTGGGGCCAGGGCACCCAGGTGAC |
| | CGTCTCGAGTGGCGGCGGATCCCAGGTGCAGCTGCAGGAGAGCGGCGGCGGCCTGGTG |
| | CAGCCCGGCGGCAGCCTGAGGCTGAGCTGCGCCGCCAGCGGCTTCACCTTCAGCCTGA |
| | GCAGCATGAGCTGGGTGAGGCAGGCCCCCGGCAAGGGCCTGGAGTGGGTGAGCGCCAT |
| | CAGCAGCGGCGGCGCCAGCACCTACTACACCGACAGCGTGAAGGGCAGGTTCACCATC |
| | AGCAGGGACAACGCCAAGAACATGCTGTACCTGCAGCTGAACAGCCTGAAGACCGAGG |
| | ACACCGCCATGTACTACTGCGCCAAGGGCGGCAGCGGCTACGGCGACGCCAGCAGGAT |
| | GACCAGCCCCGGCAGCCAGGGCACCCAGGTGACCGTGAGCGCTAGCCACCACCACCAC |
| | CACCACCACCAC |
| 137 | CAGGTGCAGCTGCAGGAGAGCGGCGGCGGCAGCGTGCAGGCCGGCGGCAGCCTGACC |
| | CTGAGCTGCGCCGCCAGCGAGTACGCCTACAGCACCTGCAACATGGGCTGGTACAGGC |
| | AGGCCCCCGGCAAGGAGAGGGAGCTGGTGAGCGCCTTCATCAGCGACGGCAGCACCTA |
| | CTACGCCGACAGCGTGAAGGGCAGGTTCACCATCACCAGGGACAACGCCAAGAACACC |
| | GTGTACCTGCAGATGAACAGCCTGAAGCCCGAGGACACCGCCATCTACTACTGCAGCG |
| | CCAACTGCTACAGGAGGCTGAGGAACTACTGGGGCCAGGGCACCCAGGTGACCGTCTC |
| | GAGTGGCGGCGGATCCCAGGTGCAGCTGCAGGAGAGCGGCGGCGGCCTGGTGCAGCCC |
| | GGCGGCAGCCTGAGGCTGAGCTGCGCCGCCAGCGGCTTCACCTTCAGCAGCTACCCCAT |
| | GAGCTGGGTGAGGCAGGCCCCCGGCAAGGGCCTGGAGTGGATCAGCACCATCAGCGCC |
| | GGCGGCGACACCACCCTGTACGCCGACAGCGTGAAGGGCAGGTTCACCAGCAGCAGGG |
| | ACAACGCCAAGAACACCCTGTACCTGCAGCTGAACAGCCTGAAGACCGAGGACGCCGC |
| | CATCTACTACTGCGCCAAGAGGATCGACTGCAACAGCGGCTACTGCTACAGGAGGAAC |
| | TACTGGGGCCAGGGCACCCAGGTGACCGTGAGCGCTAGCCACCACCACCACCACCACC |
| | ACCAC |
| 138 | CAGGTGCAGCTGCAGGAGAGCGGCGGCGGCAGCGTGCAGGCCGGCGGCAGCCTGACC |
| | CTGAGCTGCGCCGCCAGCGAGTACGCCTACAGCACCTGCAACATGGGCTGGTACAGGC. |
| | AGGCCCCCGGCAAGGAGAGGGAGCTGGTGAGCGCCTTCATCAGCGACGGCAGCACCTA |
| | CTACGCCGACAGCGTGAAGGGCAGGTTCACCATCACCAGGGACAACGCCAAGAACACC |
| | GTGTACCTGCAGATGAACAGCCTGAAGCCCGAGGACACCGCCATCTACTACTGCAGCG |
| | CCAACTGCTACAGGAGGCTGAGGAACTACTGGGGCCAGGGCACCCAGGTGACCGTCTC |
| | GAGTGGCGGCGGATCCCAGGTGCAGCTGCAGGAGAGCGGCGGGGGCCTGGTGCAGCCC |
| | GGCGAGAGCCTGAGGCTGAGCTGCACCGCCAGCGGCTTCACCTTCAGCAACTACGCCA |
| | TGAGCTGGGTGAGGCAGGCCCCCGGCAAGGGCCTGGAGTGGGTGAGCGGCATCAACGT |
| | GGCCTACGGCATCACCAGCTACGCCGACAGCGTGAAGGGCAGGTTCACCATCAGCAGG |
| | GACAACACCAAGAACACCCTGTACCTGCAGCTGAACAGCCTGAAGACCGAGGACACCG |
| | CCATCTACTACTGCGTGAAGCACAGCGGCACCACCATCCCCAGGGGCTTCATCAGCTAC |
| | ACCAAGAGGGGCCAGGGCACCCAGGTGACCGTGAGCGCTAGCCACCACCACCACCACC |
| | ACCACCAC |
| 139 | CAGGTGCAGCTGCAGGAGAGCGGCGGCGGCAGCGTGCAGGCCGGCGGCAGCCTGACC |
| | CTGAGCTGCGCCGCCAGCGAGTACGCCTACAGCACCTGCAACATGGGCTGGTACAGGC |
| | AGGCCCCCGGCAAGGAGAGGGAGCTGGTGAGCGCCTTCATCAGCGACGGCAGCACCTA |
| | CTACGCCGACAGCGTGAAGGGCAGGTTCACCATCACCAGGGACAACGCCAAGAACACC |
| | GTGTACCTGCAGATGAACAGCCTGAAGCCCGAGGACACCGCCATCTACTACTGCAGCG |
| | CCAACTGCTACAGGAGGCTGAGGAACTACTGGGGCCAGGGCACCCAGGTGACCGTCTC |
| | GAGTGGCGGCGGATCCCAGGTGCAGCTGCAGGAGAGCGGCGGCGGCCTGGTGCAGCCC |
| | GGCGGCAGCCTGAGGCTGAGCTGCGCCGCCAGCGGCTTCAGCTTCAGCAGCTACGCCA |
| | TGAAGTGGGTGAGGCAGGCCCCCGGCAAGGGCCTGGAGTGGGTGAGCACCATCAGCAG |
| | CGGCGGCAGCAGCACCAACTACGCCGACAGCGTGAAGGGCAGGTTCACCATCAGCAGG |
| | GACAACGCCAAGAACACCCTGTACCTGCAGCTGAACAGCCTGAAGATCGAGGACACCG |
| | CCATGTACTACTGCGCCAAGGCCATCGTGCCCACCGGCGCCACCATGGAGAGGGGCCA |
| | GGGCACCCAGGTGACCGTGAGCGCTAGCCACCACCACCACCACCACCACCAC |
| 140 | CAGGTGCAGCTGCAGGAGAGCGGGGGGGGCAGCGTGCAGGCCGGCGGCAGCCTGACC |
| | CTGAGCTGCGCCGCCAGCGAGTACGCCTACAGCACCTGCAACATGGGCTGGTACAGGC |
| | AGGCCCCCGGCAAGGAGAGGGAGCTGGTGAGCGCCTTCATCAGCGACGGCAGCACCTA |
| | CTACGCCGACAGCGTGAAGGGCAGGTTCACCATCACCAGGGACAACGCCAAGAACACC |
| | GTGTACCTGCAGATGAACAGCCTGAAGCCCGAGGACACCGCCATCTACTACTGCAGCG |
| | CCAACTGCTACAGGAGGCTGAGGAACTACTGGGGCCAGGGCACCCAGGTGACCGTCTC |
| | GAGTGGCGGCGGATCCCAGGTGCAGCTGCAGGAGAGCGGCGGCGGCAGCGTGCAGAG |
| | CGGCGGCAGCCTGAGGCTGAGCTGCGCCGCCAGCGGCTTCACCTACAGCACCAGCAAC |
| | AGCTGGATGGCCTGGTTCAGGCAGGCCCCCGGCAAGGAGAGGGAGGGCGTGGCCGCCA |
| | TCTACACCGTGGGGGGCAGCATCTTCTACGCCGACAGCGTGAGGGGCAGGTTCACCATC |
| | AGCCAGGACGCCACCAAGAACATGTTCTACCTGCAGATGAACACCCTGAAGCCCGAGG |
| | ACACCGCCATGTACTACTGCGCCGCCGCCAGCGGCAGGCTGAGGGGCAAGTGGTTCTG |
| | GCCCTACGAGTACAACTACTGGGGCCAGGGCACCCAGGTGACCGTGAGCGCTAGCCAC |
| | CACCACCACCACCACCACCAC |
| 141 | CAGGTGCAGCTGCAGGAGAGCGGCGGCGGCAGCGTGCAGGCCGGCGGCAGCCTGACC |
| | CTGAGCTGCGCCGCCAGCGAGTACGCCTACAGCACCTGCAACATGGGCTGGTACAGGC |
| | AGGCCCCCGGCAAGGAGAGGGAGCTGGTGAGCGCCTTCATCAGCGACGGCAGCACCTA |
| | CTACGCCGACAGCGTGAAGGGCAGGTTCACCATCACCAGGGACAACGCCAAGAACACC |
| | GTGTACCTGCAGATGAACAGCCTGAAGCCCGAGGACACCGCCATCTACTACTGCAGCG |
| | CCAACTGCTACAGGAGGCTGAGGAACTACTGGGGCCAGGGCACCCAGGTGACCGTCTC |
| | GAGTGGCGGCGGATCCCAGGTGCAGCTGCAGGAGAGCGGCGGCGGCAGCGTGCAGGC |
| | CGGCGGCAGCCTGAGGCTGAGCTGCGTGGCCAGCGGCTACGTGAGCTGCGACTACTTC |
| | CTGCCCAGCTGGTACAGGCAGGCCCCCGGCAAGGAGAGGGAGTTCGTGAGCATCATCG |
| | ACGGCACCGGCAGCACCAGCTACGCCGCCAGCGTGAAGGGCAGGTTCACCGCCAGCCA |
| | GGACAAGGGCAAGAACATCGCCTACCTGCAGATGAACACCCTGAAGCCCGAGGACACC |
| | GCCATGTACTACTGCAAGGCCAGCTGCGTGAGGGGCAGGGCCATCAGCGAGTACTGGG |
| | GCCAGGGCACCCAGGTGACCGTGAGCGCTAGCCACCACCACCACCACCACCACCAC |
| 142 | CAGGTGCAGCTGCAGGAGAGGGGGGCGGCAGCGTGCAGGCCGGCGGCAGCCTGACC |
| | CTGAGCTGCGCCGCCAGCGAGTACGCCTACAGCACCTGCAACATGGGCTGGTACAGGC |
| | AGGCCCCCGGCAAGGAGAGGGAGCTGGTGAGCGCCTTCATCAGCGACGGCAGCACCTA |
| | CTACGCCGACAGCGTGAAGGGCAGGTTCACCATCACCAGGGACAACGCCAAGAACACC |
| | GTGTACCTGCAGATGAACAGCCTGAAGCCCGAGGACACCGCCATCTACTACTGCAGCG |
| | CCAACTGCTACAGGAGGCTGAGGAACTACTGGGGCCAGGGCACCCAGGTGACCGTCTC |
| | GAGTGGCGGCGGATCCCAGGTGCAGCTGCAGGAGAGCGGCGGGGGCAGCGTGCAGGC |
| | CGGCGGCAGCCTGAGGCTGAGCTGCAGGGCCAGCGGCAGCACCTACAGCAACTACTGC |
| | CTGGGCTGGTTCAGGCAGATCACCGGCAAGGAGAGGGAGGGCGTGGCCGTGATCAACT |
| | GGGTGGGCGGCATGCTGTACTTCGCCGACAGCGTGAAGGGCAGGTTCACCGTGAGCCA |
| | GGACCAGGCCAAGAACACCGTGTACCTGCAGATGAACAGCCTGAAGCCCGAGGACACC |
| | GCCATGTACTACTGCGCCGCCGAGAGCGTGAGCAGCTTCAGCTGCGGCGGCTGGCTGA |
| | CCAGGCCCGACAGGGTGCCCTACTGGGGCCAGGGCACCCAGGTGACCGTGAGCGCTAG |
| | CCACCACCACCACCACCACCACCAC |
| 143 | CAGGTGCAGCTGCAGGAGAGCGGGGGGGGCAGCGTGCAGGCCGGCGGCAGCCTGACC |
| | CTGAGCTGCGCCGCCAGCGAGTACGCCTACAGCACCTGCAACATGGGCTGGTACAGGC |
| | AGGCCCCCGGCAAGGAGAGGGAGCTGGTGAGCGCCTTCATCAGCGACGGCAGCACCTA |
| | CTACGCCGACAGCGTGAAGGGCAGGTTCACCATCACCAGGGACAACGCCAAGAACACC |
| | GTGTACCTGCAGATGAACAGCCTGAAGCCCGAGGACACCGCCATCTACTACTGCAGCG |
| | CCAACTGCTACAGGAGGCTGAGGAACTACTGGGGCCAGGGCACCCAGGTGACCGTCTC |
| | GAGTGGCGGCGGATCCCAGGTGCAGCTGCAGGAGAGCGGCGGCGGCCTGGTGCAGCCC |
| | GGCGGCAGCCTGAGGCTGAGCTGCGCCGCCAGCGGCTTCACCTTCAGCCTGAGCAGCA |
| | TGAGCTGGGTGAGGCAGGCCCCCGGCAAGGGCCTGGAGTGGGTGAGCGCCATCAGCAG |
| | CGGCGGCGCCAGCACCTACTACACCGACAGCGTGAAGGGCAGGTTCACCATCAGCAGG |
| | GACAACGCCAAGAACATGCTGTACCTGCAGCTGAACAGCCTGAAGACCGAGGACACCG |
| | CCATGTACTACTGCGCCAAGGGCGGCAGCGGCTACGGCGACGCCAGCAGGATGACCAG |
| | CCCCGGCAGCCAGGGCACCCAGGTGACCGTGAGCGCTAGCCACCACCACCACCACCAC |
| | CACCAC |
| 144 | CAGGTGCAGCTGCAGGAGAGGGGGGGGCCTGGTGCAGCCCGGCGGCAGCCTGAGGC |
| | TGAGCTGCACCGCCAGCGGCCTGACCTTCGACGACAGCGTGATGGGCTGGTTCAGGCA |
| | GGCCCCCGGCAAGGGCAGGGAGGCCGTGAGCTGCATCAGCAGCAGCGGCGCCAACGC |
| | CTTCTACGCCGACAGCGTGAAGGGCAGGTTCACCATCAGCAGGGACAACGCCAAGAAC |
| | ACCCTGTACCTGCAGATGAACAGCCTGAAGCCCGAGGACACCGCCACCTACTACTGCA |
| | AGAGGGGCCACGCCTGCGCCGGCTACTACCCCATCCCCTACGACGACTACTGGGGCCA |
| | GGGCACCCAGGTGACCGTCTCGAGTGGCGGCGGATCCCAGGTGCAGCTGCAGGAGAGC |
| | GGCGGCGGCCTGGTGCAGCCCGGCGGCAGCCTGAGGCTGAGCTGCGCCGCCAGCGGCT |
| | TCACCTTCAGCAGCTACCCCATGAGCTGGGTGAGGCAGGCCCCCGGCAAGGGCCTGGA |
| | GTGGATCAGCACCATCAGCGCCGGCGGCGACACCACCCTGTACGCCGACAGCGTGAAG |
| | GGCAGGTTCACCAGCAGCAGGGACAACGCCAAGAACACCCTGTACCTGCAGCTGAACA |
| | GCCTGAAGACCGAGGACGCCGCCATCTACTACTGCGCCAAGAGGATCGACTGCAACAG |
| | CGGCTACTGCTACAGGAGGAACTACTGGGGCCAGGGCACCCAGGTGACCGTGAGCGCT |
| | AGCCACCACCACCACCACCACCACCAC |
| 145 | CAGGTGCAGCTGCAGGAGAGCGGCGGCGGCCTGGTGCAGCCCGGCGGCAGCCTGAGGC |
| | TGAGCTGCACCGCCAGCGGCCTGACCTTCGACGACAGCGTGATGGGCTGGTTCAGGCA |
| | GGCCCCCGGCAAGGGCAGGGAGGCCGTGAGCTGCATCAGCAGCAGCGGCGCCAACGC |
| | CTTCTACGCCGACAGCGTGAAGGGCAGGTTCACCATCAGCAGGGACAACGCCAAGAAC |
| | ACCCTGTACCTGCAGATGAACAGCCTGAAGCCCGAGGACACCGCCACCTACTACTGCA |
| | AGAGGGGCCACGCCTGCGCCGGCTACTACCCCATCCCCTACGACGACTACTGGGGCCA |
| | GGGCACCCAGGTGACCGTCTCGAGTGGGGGCGGATCCCAGGTGCAGCTGCAGGAGAGC |
| | GGCGGCGGCCTGGTGCAGCCCGGCGAGAGCCTGAGGCTGAGCTGCACCGCCAGCGGCT |
| | TCACCTTCAGCAACTACGCCATGAGCTGGGTGAGGCAGGCCCCCGGCAAGGGCCTGGA |
| | GTGGGTGAGCGGCATCAACGTGGCCTACGGCATCACCAGCTACGCCGACAGCGTGAAG |
| | GGCAGGTTCACCATCAGCAGGGACAACACCAAGAACACCCTGTACCTGCAGCTGAACA |
| | GCCTGAAGACCGAGGACACCGCCATCTACTACTGCGTGAAGCACAGCGGCACCACCAT |
| | CCCCAGGGGCTTCATCAGCTACACCAAGAGGGGCCAGGGCACCCAGGTGACCGTGAGC |
| | GCTAGCCACCACCACCACCACCACCACCAC |
| 146 | CAGGTGCAGCTGCAGGAGAGCGGCGGCGGCCTGGTGCAGCCCGGCGGCAGCCTGAGGC |
| | TGAGCTGCACCGCCAGCGGCCTGACCTTCGACGACAGCGTGATGGGCTGGTTCAGGCA |
| | GGCCCCCGGCAAGGGCAGGGAGGCCGTGAGCTGCATCAGCAGCAGCGGCGCCAACGC |
| | CTTCTACGCCGACAGCGTGAAGGGCAGGTTCACCATCAGCAGGGACAACGCCAAGAAC |
| | ACCCTGTACCTGCAGATGAACAGCCTGAAGCCCGAGGACACCGCCACCTACTACTGCA |
| | AGAGGGGCCACGCCTGCGCCGGCTACTACCCCATCCCCTACGACGACTACTGGGGCCA |
| | GGGCACCCAGGTGACCGTCTCGAGTGGCGGCGGATCCCAGGTGCAGCTGCAGGAGAGC |
| | GGGGCGGCCTGGTGCAGCCCGGCGGCAGCCTGAGGCTGAGCTGCGCCGCCAGCGGCT |
| | TCAGCTTCAGCAGCTACGCCATGAAGTGGGTGAGGCAGGCCCCCGGCAAGGGCCTGGA |
| | GTGGGTGAGCACCATCAGCAGCGGGGGCAGCAGCACCAACTACGCCGACAGCGTGAAG |
| | GGCAGGTTCACCATCAGCAGGGACAACGCCAAGAACACCCTGTACCTGCAGCTGAACA |
| | GCCTGAAGATCGAGGACACCGCCATGTACTACTGCGCCAAGGCCATCGTGCCCACCGG |
| | CGCCACCATGGAGAGGGGCCAGGGCACCCAGGTGACCGTGAGCGCTAGCCACCACCAC |
| | CACCACCACCACCAC |
| 147 | CAGGTGCAGCTGCAGGAGAGCGGCGGCGGCCTGGTGCAGCCCGGCGGCAGCCTGAGGC |
| | TGAGCTGCACCGCCAGCGGCCTGACCTTCGACGACAGCGTGATGGGCTGGTTCAGGCA |
| | GGCCCCCGGCAAGGGCAGGGAGGCCGTGAGCTGCATCAGCAGCAGCGGCGCCAACGC |
| | CTTCTACGCCGACAGCGTGAAGGGCAGGTTCACCATCAGCAGGGACAACGCCAAGAAC |
| | ACCCTGTACCTGCAGATGAACAGCCTGAAGCCCGAGGACACCGCCACCTACTACTGCA |
| | AGAGGGGCCACGCCTGCGCCGGCTACTACCCCATCCCCTACGACGACTACTGGGGCCA |
| | GGGCACCCAGGTGACCGTCTCGAGTGGGGGCGGATCCCAGGTGCAGCTGCAGGAGAGC |
| | GGCGGCGGCAGCGTGCAGAGCGGCGGCAGCCTGAGGCTGAGCTGCGCCGCCAGCGGCT |
| | TCACCTACAGCACCAGCAACAGCTGGATGGCCTGGTTCAGGCAGGCCCCCGGCAAGGA |
| | GAGGGAGGGCGTGGCCGCCATCTACACCGTGGGGGGCAGCATCTTCTACGCCGACAGC |
| | GTGAGGGGCAGGTTCACCATCAGCCAGGACGCCACCAAGAACATGTTCTACCTGCAGA |
| | TGAACACCCTGAAGCCCGAGGACACCGCCATGTACTACTGCGCCGCCGCCAGCGGCAG |
| | GCTGAGGGGCAAGTGGTTCTGGCCCTACGAGTACAACTACTGGGGCCAGGGCACCCAG |
| | GTGACCGTGAGCGCTAGCCACCACCACCACCACCACCACCAC |
| 148 | CAGGTGCAGCTGCAGGAGAGCGGCGGCGGCCTGGTGCAGCCCGGCGGCAGCCTGAGGC |
| | TGAGCTGCACCGCCAGCGGCCTGACCTTCGACGACAGCGTGATGGGCTGGTTCAGGCA |
| | GGCCCCCGGCAAGGGCAGGGAGGCCGTGAGCTGCATCAGCAGCAGCGGCGCCAACGC |
| | CTTCTACGCCGACAGCGTGAAGGGCAGGTTCACCATCAGCAGGGACAACGCCAAGAAC |
| | ACCCTGTACCTGCAGATGAACAGCCTGAAGCCCGAGGACACCGCCACCTACTACTGCA |
| | AGAGGGGCCACGCCTGCGCCGGCTACTACCCCATCCCCTACGACGACTACTGGGGCCA |
| | GGGCACCCAGGTGACCGTCTCGAGTGGCGGCGGATCCCAGGTGCAGCTGCAGGAGAGC |
| | GGCGGCGGCAGCGTGCAGGCCGGCGGCAGCCTGAGGCTGAGCTGCGTGGCCAGCGGCT |
| | ACGTGAGCTGCGACTACTTCCTGCCCAGCTGGTACAGGCAGGCCCCCGGCAAGGAGAG |
| | GGAGTTCGTGAGCATCATCGACGGCACCGGCAGCACCAGCTACGCCGCCAGCGTGAAG |
| | GGCAGGTTCACCGCCAGCCAGGACAAGGGCAAGAACATCGCCTACCTGCAGATGAACA |
| | CCCTGAAGCCCGAGGACACCGCCATGTACTACTGCAAGGCCAGCTGCGTGAGGGGCAG |
| | GGCCATCAGCGAGTACTGGGGCCAGGGCACCCAGGTGACCGTGAGCGCTAGCCACCAC |
| | CACCACCACCACCACCAC |
| 149 | CAGGTGCAGCTGCAGGAGAGCGGCGGCGGCCTGGTGCAGCCCGGCGGCAGCCTGAGGC |
| | TGAGCTGCACCGCCAGCGGCCTGACCTTCGACGACAGCGTGATGGGCTGGTTCAGGCA |
| | GGCCCCCGGCAAGGGCAGGGAGGCCGTGAGCTGCATCAGCAGCAGCGGCGCCAACGC |
| | CTTCTACGCCGACAGCGTGAAGGGCAGGTTCACCATCAGCAGGGACAACGCCAAGAAC |
| | ACCCTGTACCTGCAGATGAACAGCCTGAAGCCCGAGGACACCGCCACCTACTACTGCA |
| | AGAGGGGCCACGCCTGCGCCGGCTACTACCCCATCCCCTACGACGACTACTGGGGCCA |
| | GGGCACCCAGGTGACCGTCTCGAGTGGCGGCGGATCCCAGGTGCAGCTGCAGGAGAGC |
| | GGCGGCGGCAGCGTGCAGGCCGGCGGCAGCCTGAGGCTGAGCTGCAGGGCCAGCGGC |
| | AGCACCTACAGCAACTACTGCCTGGGCTGGTTCAGGCAGATCACCGGCAAGGAGAGGG |
| | AGGGCGTGGCCGTGATCAACTGGGGGGCGGCATGCTGTACTTCGCCGACAGCGTGAA |
| | GGGCAGGTTCACCGTGAGCCAGGACCAGGCCAAGAACACCGTGTACCTGCAGATGAAC |
| | AGCCTGAAGCCCGAGGACACCGCCATGTACTACTGCGCCGCCGAGAGCGTGAGCAGCT |
| | TCAGCTGCGGCGGCTGGCTGACCAGGCCCGACAGGGTGCCCTACTGGGGCCAGGGCAC |
| | CCAGGTGACCGTGAGCGCTAGCCACCACCACCACCACCACCACCAC |
| 150 | CAGGTGCAGCTGCAGGAGAGCGGCGGGGGCCTGGTGCAGCCCGGCGGCAGCCTGAGGC |
| | TGAGCTGCACCGCCAGCGGCCTGACCTTCGACGACAGCGTGATGGGCTGGTTCAGGCA |
| | GGCCCCCGGCAAGGGCAGGGAGGCCGTGAGCTGCATCAGCAGCAGCGGCGCCAACGC |
| | CTTCTACGCCGACAGCGTGAAGGGCAGGTTCACCATCAGCAGGGACAACGCCAAGAAC |
| | ACCCTGTACCTGCAGATGAACAGCCTGAAGCCCGAGGACACCGCCACCTACTACTGCA |
| | AGAGGGGCCACGCCTGCGCCGGCTACTACCCCATCCCCTACGACGACTACTGGGGCCA |
| | GGGCACCCAGGTGACCGTCTCGAGTGGCGGCGGATCCCAGGTGCAGCTGCAGGAGAGC |
| | GGCGGCGGCCTGGTGCAGCCCGGCGGCAGCCTGAGGCTGAGCTGCGCCGCCAGCGGCT |
| | TCACCTTCAGCCTGAGCAGCATGAGCTGGGTGAGGCAGGCCCCCGGCAAGGGCCTGGA |
| | GTGGGTGAGCGCCATCAGCAGCGGCGGCGCCAGCACCTACTACACCGACAGCGTGAAG |
| | GGCAGGTTCACCATCAGCAGGGACAACGCCAAGAACATGCTGTACCTGCAGCTGAACA |
| | GCCTGAAGACCGAGGACACCGCCATGTACTACTGCGCCAAGGGGGGCAGCGGCTACGG |
| | CGACGCCAGCAGGATGACCAGCCCCGGCAGCCAGGGCACCCAGGTGACCGTGAGCGCT |
| | AGCCACCACCACCACCACCACCACCAC |

Anti-IL27Rα V_{H}H-Linker-Anti-GP130 V_{H}H

A bispecific V_{H}H² can contain, from the N-terminus to the C-terminus, a first V_{H}H binding to IL27Rα (an anti-IL27Rα V_{H}H antibody), a linker, and a second V_{H}H binding to gp130 (an anti-gp130 V_{H}H antibody). In other words, the linker joins the C-terminus of the anti-IL27Rα V_{H}H in the binding protein to the N-terminus of the anti-gp130 V_{H}H in the binding protein. In some embodiments, a purification peptide, e.g., a six-histidine peptide ((His), (SEQ ID NO: 1531) or His-tag) or an Fe tag can be included in the bispecific V_{H}H².

In certain embodiments, a bispecific V_{H}H² described herein comprises an anti-IL27Rα V_{H}H antibody comprising a sequence having at least 90% (e.g., 91%, 92%, 93%, 94%, 95%, 96%, 97%, 98%, 99%, or 100%) sequence identity to a sequence of any one of SEQ ID NOS: 245-251; and an anti-gp130 V_{H}H antibody comprising a sequence having at least 90% (e.g., 91%, 92%, 93%, 94%, 95%, 96%, 97%, 98%, 99%, or 100%) sequence identity to a sequence of any one of SEQ ID NOS: 252-257.

In certain embodiments, a bispecific V_{H}H² described herein comprises an anti-IL27Rα V_{H}H antibody and an anti-gp130 V_{H}H antibody as described in each row of Table 3A below or Table A above. In some embodiments, in each row of Table 3A, the anti-IL27Rα V_{H}H antibody and the anti-gp130 V_{H}H antibody can each, independently, comprise at least 90% (e.g., 91%, 92%, 93%, 94%, 95%, 96%, 97%, 98%, 99%, or 100%) sequence identity to the sequence described in each row of Table 3A. In some embodiments, the bispecific V_{H}H² can comprise a linker (e.g., linkers described in Section IV) between the anti-IL27Rα V_{H}H antibody and the anti-gp130 V_{H}H antibody as described in each row of Table 3A below. In particular embodiments, the linker is GGGS (SEQ ID NO:108). The sequence of the anti-IL27Rα V_{H}H is N-terminal to the linker and the sequence of the anti-gp130 V_{H}H is C-terminal to the linker. Examples of linkers are further described in Section IV below. The CDR sequences in each V_{H}H are underlined.

TABLE 3A

| N-terminal Anti-IL27Rα V_{H}H | C-terminal Anti-gp130 V_{H}H |
|---|---|
| QVQLQESGGGLVQPGGSLRLSCAASGFTFSSY | QVQLQESGGGSVQAGGSLRLSCTASGAIASGY |
| PMSWVRQAPGKGLEWISTISAGGDTTLYADSV | IDSRWCMAWFRQAPGKEREGVAAIWPGGGLT |
| KGRFTSSRDNAKNTLYLQLNSLKTEDAAIYYC | VYADSVKGRFTISRDHAKNTLYLQMNNLKPE |
| AKRIDCNSGYCYRRNYWGQGTQVTVSS (SEQ | DTAMYYCAAGSPRMCPSLEFGFDYWGQGTQV |
| ID NO: 245) | TVS (SEQ ID NO: 252) |
| QVQLQESGGGLVQPGGSLRLSCAASGFTFSSY | QVQLQESGGGSVQAGGSLRLSCTAPGFTSNSC |
| PMSWVRQAPGKGLEWISTISAGGDTTLYADSV | GMDWYRQAPGKEREFVSSISTDGTTGYADSV |
| KGRFTSSRDNAKNTLYLQLNSLKTEDAAIYYC | KGRFTISKDKAKDTVYLQMNSLKPEDIGMYS |
| AKRIDCNSGYCYRRNYWGQGTQVTVSS (SEQ | CKTKDGTIATMELCDFGYWGQGTQVTVS |
| ID NO: 245) | (SEQ ID NO: 253) |
| QVQLQESGGGLVQPGGSLRLSCAASGFTFSSY | QVQLQESGGGSVQAGGSLRLSCAASGYPYSN |
| PMSWVRQAPGKGLEWISTISAGGDTTLYADSV | GYMGWFRQAPGKEREGVATIYTGDGRTYYAD |
| KGRFTSSRDNAKNTLYLQLNSLKTEDAAIYYC | SVKGRFTISRDNAKNTVDLQMSSLKPEDTAMY |
| AKRIDCNSGYCYRRNYWGQGTQVTVSS (SEQ | YCAARAAPLYSSGSPLTRARYNVWGQGTQVT |
| ID NO: 245) | VS (SEQ ID NO: 254) |
| QVQLQESGGGLVQPGGSLRLSCAASGFTFSSY | QVQLQESGGGSVQAGGSLRLSCVASASTYCTY |
| PMSWVRQAPGKGLEWISTISAGGDTTLYADSV | DMHWYRQAPGKGREFVSAIDSDGTTRYADSV |
| KGRFTSSRDNAKNTLYLQLNSLKTEDAAIYYC | KGRFTISQGTAKNTVYLQMNSLQPEDTAMYY |
| AKRIDCNSGYCYRRNYWGQGTQVTVSS (SEQ | CKTVCVVGSRWSDYWGQGTQVTVS (SEQ ID |
| ID NO: 245) | NO: 255) |
| QVQLQESGGGLVQPGGSLRLSCAASGFTFSSY | QVQLQESGGGSVQAGGSLILSCAASEYAYSTC |
| PMSWVRQAPGKGLEWISTISAGGDTTLYADSV | NMGWYRQAPGKERELVSAFISDGSTYYADSV |
| KGRFTSSRDNAKNTLYLQLNSLKTEDAAIYYC | KGRFTITRDNAKNTVYLQMNSLKPEDTAIYYC |
| AKRIDCNSGYCYRRNYWGQGTQVTVSS (SEQ | SANCYRRLRNYWGQGTQVTVS (SEQ ID |
| ID NO: 245) | NO: 256) |
| QVQLQESGGGLVQPGGSLRLSCAASGFTFSSY | QVQLQESGGGLVQPGGSLRLSCTASGLTFDDS |
| PMSWVRQAPGKGLEWISTISAGGDTTLYADSV | VMGWFRQAPGKGREAVSCISSSGANAFYADS |
| KGRFTSSRDNAKNTLYLQLNSLKTEDAAIYYC | VKGRFTISRDNAKNTLYLQMNSLKPEDTATYY |
| AKRIDCNSGYCYRRNYWGQGTQVTVSS (SEQ | CKRGHACAGYYPIPYDDYWGQGTQVTVS |
| ID NO: 245) | (SEQ ID NO: 257) |
| QVQLQESGGGLVQPGESLRLSCTASGFTFSNY | QVQLQESGGGSVQAGGSLRLSCTASGAIASGY |
| AMSWVRQAPGKGLEWVSGINVAYGITSYADS | IDSRWCMAWFRQAPGKEREGVAAIWPGGGLT |
| VKGRFTISRDNIKNTLYLQLNSLKTEDTAIYYC | VYADSVKGRFTISRDHAKNTLYLQMNNLKPE |
| VKHSGTTIPRGFISYTKRGQGTQVTVSS (SEQ | DTAMYYCAAGSPRMCPSLEFGFDYWGQGTQV |
| ID NO: 246) | TVS (SEQ ID NO: 252) |
| QVQLQESGGGLVQPGESLRLSCTASGFTFSNY | QVQLQESGGGSVQAGGSLRLSCTAPGFTSNSC |
| AMSWVRQAPGKGLEWVSGINVAYGITSYADS | GMDWYRQAPGKEREFVSSISTDGTTGYADSV |
| VKGRFTISRDNIKNTLYLQLNSLKTEDTAIYYC | KGRFTISKDKAKDTVYLQMNSLKPEDIGMYS |
| VKHSGTTIPRGFISYTKRGQGTQVTVSS (SEQ | CKTKDGTIATMELCDFGYWGQGTQVTVS |
| ID NO: 246) | (SEQ ID NO: 253) |
| QVQLQESGGGLVQPGESLRLSCTASGFTFSNY | QVQLQESGGGSVQAGGSLRLSCAASGYPYSN |
| AMSWVRQAPGKGLEWVSGINVAYGITSYADS | GYMGWFRQAPGKEREGVATIYTGDGRTYYAD |
| VKGRFTISRDNIKNTLYLQLNSLKTEDTAIYYC | SVKGRFTISRDNAKNTVDLQMSSLKPEDTAMY |
| VKHSGTTIPRGFISYTKRGQGTQVTVSS (SEQ | YCAARAAPLYSSGSPLTRARYNVWGQGTQVT |
| ID NO: 246) | VS (SEQ ID NO: 254) |
| QVQLQESGGGLVQPGESLRLSCTASGFTFSNY | QVQLQESGGGSVQAGGSLRLSCVASASTYCTY |
| AMSWVRQAPGKGLEWVSGINVAYGITSYADS | DMHWYRQAPGKGREFVSAIDSDGTTRYADSV |
| VKGRFTISRDNIKNTLYLQLNSLKTEDTAIYYC | KGRFTISQGTAKNTVYLQMNSLQPEDTAMYY |
| VKHSGTTIPRGFISYTKRGQGTQVTVSS (SEQ | CKTVCVVGSRWSDYWGQGTQVTVS (SEQ ID |
| ID NO: 246) | NO: 255) |
| QVQLQESGGGLVQPGESLRLSCTASGFTFSNY | QVQLQESGGGSVQAGGSLILSCAASEYAYSTC |
| AMSWVRQAPGKGLEWVSGINVAYGITSYADS | NMGWYRQAPGKERELVSAFISDGSTYYADSV |
| VKGRFTISRDNIKNTLYLQLNSLKTEDTAIYYC | KGRFTITRDNAKNTVYLQMNSLKPEDTAIYYC |
| VKHSGTTIPRGFISYTKRGQGTQVTVSS (SEQ | SANCYRRLRNYWGQGTQVTVS (SEQ ID |
| ID NO: 246) | NO: 256) |
| QVQLQESGGGLVQPGESLRLSCTASGFTFSNY | QVQLQESGGGLVQPGGSLRLSCTASGLTFDDS |
| AMSWVRQAPGKGLEWVSGINVAYGITSYADS | VMGWFRQAPGKGREAVSCISSSGANAFYADS |
| VKGRFTISRDNIKNTLYLQLNSLKTEDTAIYYC | VKGRFTISRDNAKNTLYLQMNSLKPEDTATYY |
| VKHSGTTIPRGFISYTKRGQGTQVTVSS (SEQ | CKRGHACAGYYPIPYDDYWGQGTQVTVS |
| ID NO: 246) | (SEQ ID NO: 257) |
| QVQLQESGGGLVQPGGSLRLSCAASGFSESSY | QVQLQESGGGSVQAGGSLRLSCTASGAIASGY |
| AMKWVRQAPGKGLEWVSTISSGGSSTNYADS | IDSRWCMAWFRQAPGKEREGVAAIWPGGGLT |
| VKGRFTISRDNAKNTLYLQLNSLKIEDTAMYY | VYADSVKGRFTISRDHAKNTLYLQMNNLKPE |
| CAKAIVPTGATMERGQGTQVTVSS (SEQ ID | DTAMYYCAAGSPRMCPSLEFGFDYWGQGTQV |
| NO: 247) | TVS (SEQ ID NO: 252) |
| QVQLQESGGGLVQPGGSLRLSCAASGFSESSY | QVQLQESGGGSVQAGGSLRLSCTAPGFTSNSC |
| AMKWVRQAPGKGLEWVSTISSGGSSTNYADS | GMDWYRQAPGKEREFVSSISTDGTTGYADSV |
| VKGRFTISRDNAKNTLYLQLNSLKIEDTAMYY | KGRFTISKDKAKDTVYLQMNSLKPEDIGMYS |
| CAKAIVPTGATMERGQGTQVTVSS (SEQ ID | CKTKDGTIATMELCDFGYWGQGTQVTVS |
| NO: 247) | (SEQ ID NO: 253) |
| QVQLQESGGGLVQPGGSLRLSCAASGFSESSY | QVQLQESGGGSVQAGGSLRLSCAASGYPYSN |
| AMKWVRQAPGKGLEWVSTISSGGSSTNYADS | GYMGWFRQAPGKEREGVATIYTGDGRTYYAD |
| VKGRFTISRDNAKNTLYLQLNSLKIEDTAMYY | SVKGRFTISRDNAKNTVDLQMSSLKPEDTAMY |
| CAKAIVPTGATMERGQGTQVTVSS (SEQ ID | YCAARAAPLYSSGSPLTRARYNVWGQGTQVT |
| NO: 247) | VS (SEQ ID NO: 254) |
| QVQLQESGGGLVQPGGSLRLSCAASGFSESSY | QVQLQESGGGSVQAGGSLRLSCVASASTYCTY |
| AMKWVRQAPGKGLEWVSTISSGGSSTNYADS | DMHWYRQAPGKGREFVSAIDSDGTTRYADSV |
| VKGRFTISRDNAKNTLYLQLNSLKIEDTAMYY | KGRFTISQGTAKNTVYLQMNSLQPEDTAMYY |
| CAKAIVPTGATMERGQGTQVTVSS (SEQ ID | CKTVCVVGSRWSDYWGQGTQVTVS (SEQ ID |
| NO: 247) | NO: 255) |
| QVQLQESGGGLVQPGGSLRLSCAASGFSESSY | QVQLQESGGGSVQAGGSLILSCAASEYAYSTC |
| AMKWVRQAPGKGLEWVSTISSGGSSTNYADS | NMGWYRQAPGKERELVSAFISDGSTYYADSV |
| VKGRFTISRDNAKNTLYLQLNSLKIEDTAMYY | KGRFTITRDNAKNTVYLQMNSLKPEDTAIYYC |
| CAKAIVPTGATMERGQGTQVTVSS (SEQ ID | SANCYRRLRNYWGQGTQVTVS (SEQ ID |
| NO: 247) | NO: 256) |
| QVQLQESGGGLVQPGGSLRLSCAASGFSESSY | QVQLQESGGGLVQPGGSLRLSCTASGLTFDDS |
| AMKWVRQAPGKGLEWVSTISSGGSSTNYADS | VMGWFRQAPGKGREAVSCISSSGANAFYADS |
| VKGRFTISRDNAKNTLYLQLNSLKIEDTAMYY | VKGRFTISRDNAKNTLYLQMNSLKPEDTATYY |
| CAKAIVPTGATMERGQGTQVTVSS (SEQ ID | CKRGHACAGYYPIPYDDYWGQGTQVTVS |
| NO: 247) | (SEQ ID NO: 257) |
| QVQLQESGGGSVQSGGSLRLSCAASGFTYSTS | QVQLQESGGGSVQAGGSLRLSCTASGAIASGY |
| NSWMAWFRQAPGKEREGVAAIYTVGGSIFYA | IDSRWCMAWFRQAPGKEREGVAAIWPGGGLT |
| DSVRGRFTISQDATKNMFYLQMNTLKPEDTA | VYADSVKGRFTISRDHAKNTLYLQMNNLKPE |
| MYYCAAASGRLRGKWFWPYEYNYWGQGTQ | DTAMYYCAAGSPRMCPSLEFGFDYWGQGTQV |
| VTVSS (SEQ ID NO: 248) | TVS (SEQ ID NO: 252) |
| QVQLQESGGGSVQSGGSLRLSCAASGFTYSTS | QVQLQESGGGSVQAGGSLRLSCTAPGFTSNSC |
| NSWMAWFRQAPGKEREGVAAIYTVGGSIFYA | GMDWYRQAPGKEREFVSSISTDGTTGYADSV |
| DSVRGRFTISQDATKNMFYLQMNTLKPEDTA | KGRFTISKDKAKDTVYLQMNSLKPEDIGMYS |
| MYYCAAASGRLRGKWFWPYEYNYWGQGTQ | CKTKDGTIATMELCDFGYWGQGTQVTVS |
| VTVSS (SEQ ID NO: 248) | (SEQ ID NO: 253) |
| QVQLQESGGGSVQSGGSLRLSCAASGFTYSTS | QVQLQESGGGSVQAGGSLRLSCAASGYPYSN |
| NSWMAWFRQAPGKEREGVAAIYTVGGSIFYA | GYMGWFRQAPGKEREGVATIYTGDGRTYYAD |
| DSVRGRFTISQDATKNMFYLQMNTLKPEDTA | SVKGRFTISRDNAKNTVDLQMSSLKPEDTAMY |
| MYYCAAASGRLRGKWFWPYEYNYWGQGTQ | YCAARAAPLYSSGSPLTRARYNVWGQGTQVT |
| VTVSS (SEQ ID NO: 248) | VS (SEQ ID NO: 254) |
| QVQLQESGGGSVQSGGSLRLSCAASGFTYSTS | QVQLQESGGGSVQAGGSLRLSCVASASTYCTY |
| NSWMAWFRQAPGKEREGVAAIYTVGGSIFYA | DMHWYRQAPGKGREFVSAIDSDGTTRYADSV |
| DSVRGRFTISQDATKNMFYLQMNTLKPEDTA | KGRFTISQGTAKNTVYLQMNSLQPEDTAMYY |
| MYYCAAASGRLRGKWFWPYEYNYWGQGTQ | CKTVCVVGSRWSDYWGQGTQVTVS (SEQ ID |
| VTVSS (SEQ ID NO: 248) | NO: 255) |
| QVQLQESGGGSVQSGGSLRLSCAASGFTYSTS | QVQLQESGGGSVQAGGSLILSCAASEYAYSTC |
| NSWMAWFRQAPGKEREGVAAIYTVGGSIFYA | NMGWYRQAPGKERELVSAFISDGSTYYADSV |
| DSVRGRFTISQDATKNMFYLQMNTLKPEDTA | KGRFTITRDNAKNTVYLQMNSLKPEDTAIYYC |
| MYYCAAASGRLRGKWFWPYEYNYWGQGTQ | SANCYRRLRNYWGQGTQVTVS (SEQ ID |
| VTVSS (SEQ ID NO: 248) | NO: 256) |
| QVQLQESGGGSVQSGGSLRLSCAASGFTYSTS | QVQLQESGGGLVQPGGSLRLSCTASGLTFDDS |
| NSWMAWFRQAPGKEREGVAAIYTVGGSIFYA | VMGWFRQAPGKGREAVSCISSSGANAFYADS |
| DSVRGRFTISQDATKNMFYLQMNTLKPEDTA | VKGRFTISRDNAKNTLYLQMNSLKPEDTATYY |
| MYYCAAASGRLRGKWFWPYEYNYWGQGTQ | CKRGHACAGYYPIPYDDYWGQGTQVTVS |
| VTVSS (SEQ ID NO: 248) | (SEQ ID NO: 257) |
| QVQLQESGGGSVQAGGSLRLSCVASGYVSCD | QVQLQESGGGSVQAGGSLRLSCTASGAIASGY |
| YFLPSWYRQAPGKEREFVSIIDGTGSTSYAASV | IDSRWCMAWFRQAPGKEREGVAAIWPGGGLT |
| KGRFTASQDKGKNIAYLQMNTLKPEDTAMYY | VYADSVKGRFTISRDHAKNTLYLQMNNLKPE |
| CKASCVRGRAISEYWGQGTQVTVSS (SEQ ID | DTAMYYCAAGSPRMCPSLEFGFDYWGQGTQV |
| NO: 249) | TVS (SEQ ID NO: 252) |
| QVQLQESGGGSVQAGGSLRLSCVASGYVSCD | QVQLQESGGGSVQAGGSLRLSCTAPGFTSNSC |
| YFLPSWYRQAPGKEREFVSIIDGTGSTSYAASV | GMDWYRQAPGKEREFVSSISTDGTTGYADSV |
| KGRFTASQDKGKNIAYLQMNTLKPEDTAMYY | KGRFTISKDKAKDTVYLQMNSLKPEDIGMYS |
| CKASCVRGRAISEYWGQGTQVTVSS (SEQ ID | CKTKDGTIATMELCDFGYWGQGTQVTVS |
| NO: 249) | (SEQ ID NO: 253) |
| QVQLQESGGGSVQAGGSLRLSCVASGYVSCD | QVQLQESGGGSVQAGGSLRLSCAASGYPYSN |
| YFLPSWYRQAPGKEREFVSIIDGTGSTSYAASV | GYMGWFRQAPGKEREGVATIYTGDGRTYYAD |
| KGRFTASQDKGKNIAYLQMNTLKPEDTAMYY | SVKGRFTISRDNAKNTVDLQMSSLKPEDTAMY |
| CKASCVRGRAISEYWGQGTQVTVSS (SEQ ID | YCAARAAPLYSSGSPLTRARYNVWGQGTQVT |
| NO: 249) | VS (SEQ ID NO: 254) |
| QVQLQESGGGSVQAGGSLRLSCVASGYVSCD | QVQLQESGGGSVQAGGSLRLSCVASASTYCTY |
| YFLPSWYRQAPGKEREFVSIIDGTGSTSYAASV | DMHWYRQAPGKGREFVSAIDSDGTTRYADSV |
| KGRFTASQDKGKNIAYLQMNTLKPEDTAMYY | KGRFTISQGTAKNTVYLQMNSLQPEDTAMYY |
| CKASCVRGRAISEYWGQGTQVTVSS (SEQ ID | CKTVCVVGSRWSDYWGQGTQVTVS (SEQ ID |
| NO: 249) | NO: 255) |
| QVQLQESGGGSVQAGGSLRLSCVASGYVSCD | QVQLQESGGGSVQAGGSLILSCAASEYAYSTC |
| YFLPSWYRQAPGKEREFVSIIDGTGSTSYAASV | NMGWYRQAPGKERELVSAFISDGSTYYADSV |
| KGRFTASQDKGKNIAYLQMNTLKPEDTAMYY | KGRFTITRDNAKNTVYLQMNSLKPEDTAIYYC |
| CKASCVRGRAISEYWGQGTQVTVSS (SEQ ID | SANCYRRLRNYWGQGTQVTVS (SEQ ID |
| NO: 249) | NO: 256) |
| QVQLQESGGGSVQAGGSLRLSCVASGYVSCD | QVQLQESGGGLVQPGGSLRLSCTASGLTFDDS |
| YFLPSWYRQAPGKEREFVSIIDGTGSTSYAASV | VMGWFRQAPGKGREAVSCISSSGANAFYADS |
| KGRFTASQDKGKNIAYLQMNTLKPEDTAMYY | VKGRFTISRDNAKNTLYLQMNSLKPEDTATYY |
| CKASCVRGRAISEYWGQGTQVTVSS (SEQ ID | CKRGHACAGYYPIPYDDYWGQGTQVTVS |
| NO: 249) | (SEQ ID NO: 257) |
| QVQLQESGGGSVQAGGSLRLSCRASGSTYSNY | QVQLQESGGGSVQAGGSLRLSCTASGAIASGY |
| CLGWFRQITGKEREGVAVINWVGGMLYFADS | IDSRWCMAWFRQAPGKEREGVAAIWPGGGLT |
| VKGRFTVSQDQAKNTVYLQMNSLKPEDTAMY | VYADSVKGRFTISRDHAKNTLYLQMNNLKPE |
| YCAAESVSSFSCGGWLTRPDRVPYWGQGTQV | DTAMYYCAAGSPRMCPSLEFGFDYWGQGTQV |
| TVSS (SEQ ID NO: 250) | TVS (SEQ ID NO: 252) |
| QVQLQESGGGSVQAGGSLRLSCRASGSTYSNY | QVQLQESGGGSVQAGGSLRLSCTAPGFTSNSC |
| CLGWFRQITGKEREGVAVINWVGGMLYFADS | GMDWYRQAPGKEREFVSSISTDGTTGYADSV |
| VKGRFTVSQDQAKNTVYLQMNSLKPEDTAMY | KGRFTISKDKAKDTVYLQMNSLKPEDIGMYS |
| YCAAESVSSFSCGGWLTRPDRVPYWGQGTQV | CKTKDGTIATMELCDFGYWGQGTQVTVS |
| TVSS (SEQ ID NO: 250) | (SEQ ID NO: 253) |
| QVQLQESGGGSVQAGGSLRLSCRASGSTYSNY | QVQLQESGGGSVQAGGSLRLSCAASGYPYSN |
| CLGWFRQITGKEREGVAVINWVGGMLYFADS | GYMGWFRQAPGKEREGVATIYTGDGRTYYAD |
| VKGRFTVSQDQAKNTVYLQMNSLKPEDTAMY | SVKGRFTISRDNAKNTVDLQMSSLKPEDTAMY |
| YCAAESVSSFSCGGWLTRPDRVPYWGQGTQV | YCAARAAPLYSSGSPLTRARYNVWGQGTQVT |
| TVSS (SEQ ID NO: 250) | VS (SEQ ID NO: 254) |
| QVQLQESGGGSVQAGGSLRLSCRASGSTYSNY | QVQLQESGGGSVQAGGSLRLSCVASASTYCTY |
| CLGWFRQITGKEREGVAVINWVGGMLYFADS | DMHWYRQAPGKGREFVSAIDSDGTTRYADSV |
| VKGRFTVSQDQAKNTVYLQMNSLKPEDTAMY | KGRFTISQGTAKNTVYLQMNSLQPEDTAMYY |
| YCAAESVSSFSCGGWLTRPDRVPYWGQGTQV | CKTVCVVGSRWSDYWGQGTQVTVS (SEQ ID |
| TVSS (SEQ ID NO: 250) | NO: 255) |
| QVQLQESGGGSVQAGGSLRLSCRASGSTYSNY | QVQLQESGGGSVQAGGSLILSCAASEYAYSTC |
| CLGWFRQITGKEREGVAVINWVGGMLYFADS | NMGWYRQAPGKERELVSAFISDGSTYYADSV |
| VKGRFTVSQDQAKNTVYLQMNSLKPEDTAMY | KGRFTITRDNAKNTVYLQMNSLKPEDTAIYYC |
| YCAAESVSSFSCGGWLTRPDRVPYWGQGTQV | SANCYRRLRNYWGQGTQVTVS (SEQ ID |
| TVSS (SEQ ID NO: 250) | NO: 256) |
| QVQLQESGGGSVQAGGSLRLSCRASGSTYSNY | QVQLQESGGGLVQPGGSLRLSCTASGLTFDDS |
| CLGWFRQITGKEREGVAVINWVGGMLYFADS | VMGWFRQAPGKGREAVSCISSSGANAFYADS |
| VKGRFTVSQDQAKNTVYLQMNSLKPEDTAMY | VKGRFTISRDNAKNTLYLQMNSLKPEDTATYY |
| YCAAESVSSFSCGGWLTRPDRVPYWGQGTQV | CKRGHACAGYYPIPYDDYWGQGTQVTVS |
| TVSS (SEQ ID NO: 250) | (SEQ ID NO: 257) |
| QVQLQESGGGLVQPGGSLRLSCAASGFTFSLSS | QVQLQESGGGSVQAGGSLRLSCTASGAIASGY |
| MSWVRQAPGKGLEWVSAISSGGASTYYTDSV | IDSRWCMAWFRQAPGKEREGVAAIWPGGGLT |
| KGRFTISRDNAKNMLYLQLNSLKTEDTAMYY | VYADSVKGRFTISRDHAKNTLYLQMNNLKPE |
| CAKGGSGYGDASRMTSPGSQGTQVTVSS (SEQ | DTAMYYCAAGSPRMCPSLEFGFDYWGQGTQV |
| ID NO: 251) | TVS (SEQ ID NO: 252) |
| QVQLQESGGGLVQPGGSLRLSCAASGFTFSLSS | QVQLQESGGGSVQAGGSLRLSCTAPGFTSNSC |
| MSWVRQAPGKGLEWVSAISSGGASTYYTDSV | GMDWYRQAPGKEREFVSSISTDGTTGYADSV |
| KGRFTISRDNAKNMLYLQLNSLKTEDTAMYY | KGRFTISKDKAKDTVYLQMNSLKPEDIGMYS |
| CAKGGSGYGDASRMTSPGSQGTQVTVSS (SEQ | CKTKDGTIATMELCDFGYWGQGTQVTVS |
| ID NO: 251) | (SEQ ID NO: 253) |
| QVQLQESGGGLVQPGGSLRLSCAASGFTFSLSS | QVQLQESGGGSVQAGGSLRLSCAASGYPYSN |
| MSWVRQAPGKGLEWVSAISSGGASTYYTDSV | GYMGWFRQAPGKEREGVATIYTGDGRTYYAD |
| KGRFTISRDNAKNMLYLQLNSLKTEDTAMYY | SVKGRFTISRDNAKNTVDLQMSSLKPEDTAMY |
| CAKGGSGYGDASRMTSPGSQGTQVTVSS (SEQ | YCAARAAPLYSSGSPLTRARYNVWGQGTQVT |
| ID NO: 251) | VS (SEQ ID NO: 254) |
| QVQLQESGGGLVQPGGSLRLSCAASGFTFSLSS | QVQLQESGGGSVQAGGSLRLSCVASASTYCTY |
| MSWVRQAPGKGLEWVSAISSGGASTYYTDSV | DMHWYRQAPGKGREFVSAIDSDGTTRYADSV |
| KGRFTISRDNAKNMLYLQLNSLKTEDTAMYY | KGRFTISQGTAKNTVYLQMNSLQPEDTAMYY |
| CAKGGSGYGDASRMTSPGSQGTQVTVSS (SEQ | CKTVCVVGSRWSDYWGQGTQVTVS (SEQ ID |
| ID NO: 251) | NO: 255) |
| QVQLQESGGGLVQPGGSLRLSCAASGFTFSLSS | QVQLQESGGGSVQAGGSLILSCAASEYAYSTC |
| MSWVRQAPGKGLEWVSAISSGGASTYYTDSV | NMGWYRQAPGKERELVSAFISDGSTYYADSV |
| KGRFTISRDNAKNMLYLQLNSLKTEDTAMYY | KGRFTITRDNAKNTVYLQMNSLKPEDTAIYYC |
| CAKGGSGYGDASRMTSPGSQGTQVTVSS (SEQ | SANCYRRLRNYWGQGTQVTVS (SEQ ID |
| ID NO: 251) | NO: 256) |
| QVQLQESGGGLVQPGGSLRLSCAASGFTFSLSS | QVQLQESGGGLVQPGGSLRLSCTASGLTFDDS |
| MSWVRQAPGKGLEWVSAISSGGASTYYTDSV | VMGWFRQAPGKGREAVSCISSSGANAFYADS |
| KGRFTISRDNAKNMLYLQLNSLKTEDTAMYY | VKGRFTISRDNAKNTLYLQMNSLKPEDTATYY |
| CAKGGSGYGDASRMTSPGSQGTQVTVSS (SEQ | CKRGHACAGYYPIPYDDYWGQGTQVTVS |
| ID NO: 251) | (SEQ ID NO: 257) |

In certain embodiments, the bispecific V_{H}H² comprises a sequence that is substantially identical to a sequence of any one of SEQ ID NOS: 43-84. Such a bispecific V_{H}H² can have a sequence having at least 90% (e.g., 91%, 92%, 93%, 94%, 95%, 96%, 97%, 98%, 99%, or 100%) sequence identity to the sequence of any one of SEQ ID NOS: 43-84, as shown in Table 3B below. In each of sequences of SEQ ID NOS: 43-84, the linker GGGS (SEQ ID NO: 108) is in bold. The sequence of the anti-IL27Rα V_{H}H is N-terminus to the linker and the sequence of the anti-gp130 V_{H}H is C-terminus to the linker. The CDR sequences in each V_{H}H are underlined.

TABLE 3B

| SEQ | |
|---|---|
| NO | Sequence |
| 43 | QVQLQESGGGLVQPGGSLRLSCAASGFTFSSYPMSWVRQAPGKGLEWISTISAGGDTTLYA |
| | DSVKGRFTSSRDNAKNTLYLQLNSLKTEDAAIYYCAKRIDCNSGYCYRRNYWGQGTQVTV |
| | SSGGGSQVQLQESGGGSVQAGGSLRLSCTASGAIASGYIDSRWCMAWFRQAPGKEREGVA |
| | AIWPGGGLIVYADSVKGRFTISRDHAKNTLYLQMNNLKPEDTAMYYCAAGSPRMCPSLEF |
| | GFDYWGQGTQVTVS |
| 44 | QVQLQESGGGLVQPGGSLRLSCAASGFTFSSYPMSWVRQAPGKGLEWISTISAGGDTILYA |
| | DSVKGRFTSSRDNAKNTLYLQLNSLKTEDAAIYYCAKRIDCNSGYCYRRNYWGQGTQVTV |
| | SSGGGSQVQLQESGGGSVQAGGSLRLSCTAPGFTSNSCGMDWYRQAPGKEREFVSSISTDG |
| | TTGYADSVKGRFTISKDKAKDTVYLQMNSLKPEDIGMYSCKTKDGTIATMELCDFGYWG |
| | QGTQVTVS |
| 45 | QVQLQESGGGLVQPGGSLRLSCAASGFTFSSYPMSWVRQAPGKGLEWISTISAGGDTILYA |
| | DSVKGRFTSSRDNAKNTLYLQLNSLKTEDAAIYYCAKRIDCNSGYCYRRNYWGQGTQVTV |
| | SSGGGSQVQLQESGGGSVQAGGSLRLSCAASGYPYSNGYMGWFRQAPGKEREGVATIYT |
| | GDGRTYYADSVKGRFTISRDNAKNTVDLQMSSLKPEDTAMYYCAARAAPLYSSGSPLTRA |
| | RYNVWGQGTQVTVS |
| 46 | QVQLQESGGGLVQPGGSLRLSCAASGFTFSSYPMSWVRQAPGKGLEWISTISAGGDTTLYA |
| | DSVKGRFTSSRDNAKNTLYLQLNSLKTEDAAIYYCAKRIDCNSGYCYRRNYWGQGTQVTV |
| | SSGGGSQVQLQESGGGSVQAGGSLRLSCVASASTYCTYDMHWYRQAPGKGREFVSAIDSD |
| | GTTRYADSVKGRFTISQGTAKNTVYLQMNSLQPEDTAMYYCKTVCVVGSRWSDYWGQGT |
| | QVTVS |
| 47 | QVQLQESGGGLVQPGGSLRLSCAASGFTFSSYPMSWVRQAPGKGLEWISTISAGGDTTLYA |
| | DSVKGRFTSSRDNAKNILYLQLNSLKTEDAAIYYCAKRIDCNSGYCYRRNYWGQGTQVTV |
| | SSGGGSQVQLQESGGGSVQAGGSLILSCAASEYAYSTQNMGWYRQAPGKERELVSAFISD |
| | GSTYYADSVKGRFTITRDNAKNTVYLQMNSLKPEDTAIYYCSANCYRRLRNYWGQGTQVT |
| | VS |
| 48 | QVQLQESGGGLVQPGGSLRLSCAASGFTFSSYPMSWVRQAPGKGLEWISTISAGGDTTLYA |
| | DSVKGRFTSSRDNAKNILYLQLNSLKTEDAAIYYCAKRIDCNSGYCYRRNYWGQGTQVTV |
| | SSGGGSQVQLQESGGGLVQPGGSLRLSCTASGLTFDDSVMGWFRQAPGKGREAVSCISSSG |
| | ANAFYADSVKGRFTISRDNAKNTLYLQMNSLKPEDTATYYCKRGHACAGYYPIPYDDYW |
| | GQGTQVTVS |
| 49 | QVQLQESGGGLVQPGESLRLSCTASGFTFSNYAMSWVRQAPGKGLEWVSGINVAYGITSY |
| | ADSVKGRFTISRDNIKNTLYLQLNSLKTEDTAIYYCVKHSGTTIPRGFISYTKRGQGTQVTV |
| | SSGGGSQVQLQESGGGSVQAGGSLRLSCTASGAIASGYIDSRWCMAWFRQAPGKEREGVA |
| | AIWPGGGLIVYADSVKGRFTISRDHAKNTLYLQMNNLKPEDTAMYYCAAGSPRMCPSLEF |
| | GFDYWGQGTQVTVS |
| 50 | QVQLQESGGGLVQPGESLRLSCTASGFTFSNYAMSWVRQAPGKGLEWVSGINVAYGITSY |
| | ADSVKGRFTISRDNTKNTLYLQLNSLKTEDTAIYYCVKHSGTTIPRGFISYTKRGQGTQVTV |
| | SSGGGSQVQLQESGGGSVQAGGSLRLSCTAPGFTSNSCGMDWYRQAPGKEREFVSSISTDG |
| | TTGYADSVKGRFTISKDKAKDTVYLQMNSLKPEDIGMYSCKTKDGTIATMELCDFGYWG |
| | QGTQVTVS |
| 51 | QVQLQESGGGLVQPGESLRLSCTASGFTFSNYAMSWVRQAPGKGLEWVSGINVAYGITSY |
| | ADSVKGRFTISRDNTKNTLYLQLNSLKTEDTAIYYCVKHSGTTIPRGFISYTKRGQGTQVTV |
| | SSGGGSQVQLQESGGGSVQAGGSLRLSCAASGYPYSNGYMGWFRQAPGKEREGVATIYT |
| | GDGRTYYADSVKGRFTISRDNAKNTVDLQMSSLKPEDTAMYYCAARAAPLYSSGSPLTRA |
| | RYNVWGQGTQVTVS |
| 52 | QVQLQESGGGLVQPGESLRLSCTASGFTFSNYAMSWVRQAPGKGLEWVSGINVAYGITSY |
| | ADSVKGRFTISRDNTKNTLYLQLNSLKTEDTAIYYCVKHSGTTIPRGFISYTKRGQGTQVTV |
| | SSGGGSQVQLQESGGGSVQAGGSLRLSCVASASTYCTYDMHWYRQAPGKGREFVSAIDSD |
| | GTTRYADSVKGRFTISQGTAKNTVYLQMNSLQPEDTAMYYCKTVCVVGSRWSDYWGQGT |
| | QVTVS |
| 53 | QVQLQESGGGLVQPGESLRLSCTASGFTFSNYAMSWVRQAPGKGLEWVSGINVAYGITSY |
| | ADSVKGRFTISRDNIKNTLYLQLNSLKTEDTAIYYCVKHSGTTIPRGFISYTKRGQGTQVTV |
| | SSGGGSQVQLQESGGGSVQAGGSLTLSCAASEYAYSTCNMGWYRQAPGKERELVSAFISD |
| | GSTYYADSVKGRFTITRDNAKNTVYLQMNSLKPEDTAIYYCSANCYRRLRNYWGQGTQVT |
| | VS |
| 54 | QVQLQESGGGLVQPGESLRLSCTASGFTFSNYAMSWVRQAPGKGLEWVSGINVAYGITSY |
| | ADSVKGRFTISRDNIKNTLYLQLNSLKTEDTAIYYCVKHSGTTIPRGFISYTKRGQGTQVTV |
| | SSGGGSQVQLQESGGGLVQPGGSLRLSCTASGLTFDDSVMGWFRQAPGKGREAVSCISSSG |
| | ANAFYADSVKGRFTISRDNAKNTLYLQMNSLKPEDTATYYCKRGHACAGYYPIPYDDYW |
| | GQGTQVTVS |
| 55 | QVQLQESGGGLVQPGGSLRLSCAASGFSFSSYAMKWVRQAPGKGLEWVSTISSGGSSTNY |
| | ADSVKGRFTISRDNAKNTLYLQLNSLKIEDTAMYYCAKAIVPTGATMERGQGTQVTVSSG |
| | GGSQVQLQESGGGSVQAGGSLRLSCTASGAIASGYIDSRWCMAWFRQAPGKEREGVAAIW |
| | PGGGLTVYADSVKGRFTISRDHAKNTLYLQMNNLKPEDTAMYYCAAGSPRMCPSLEFGFD |
| | YWGQGTQVTVS |
| 56 | QVQLQESGGGLVQPGGSLRLSCAASGFSFSSYAMKWVRQAPGKGLEWVSTISSGGSSTNY |
| | ADSVKGRFTISRDNAKNTLYLQLNSLKIEDTAMYYCAKAIVPTGATMERGQGTQVTVSSG |
| | GGSQVQLQESGGGSVQAGGSLRLSCTAPGFTSNSCGMDWYRQAPGKEREFVSSISIDGTTG |
| | YADSVKGRFTISKDKAKDTVYLQMNSLKPEDIGMYSCKTKDGTIATMELCDFGYWGQGT |
| | QVTVS |
| 57 | QVQLQESGGGLVQPGGSLRLSCAASGFSFSSYAMKWVRQAPGKGLEWVSTISSGGSSTNY |
| | ADSVKGRFTISRDNAKNTLYLQLNSLKIEDTAMYYCAKAIVPTGATMERGQGTQVTVSSG |
| | GGSQVQLQESGGGSVQAGGSLRLSCAASGYPYSNGYMGWFRQAPGKEREGVATIYTGDG |
| | RTYYADSVKGRFTISRDNAKNTVDLQMSSLKPEDTAMYYCAARAAPLYSSGSPLTRARYN |
| | VWGQGTQVTVS |
| 58 | QVQLQESGGGLVQPGGSLRLSCAASGFSFSSYAMKWVRQAPGKGLEWVSTISSGGSSTNY |
| | ADSVKGRFTISRDNAKNTLYLQLNSLKIEDTAMYYCAKAIVPTGATMERGQGTQVTVSSG |
| | GGSQVQLQESGGGSVQAGGSLRLSCVASASTYCTYDMHWYRQAPGKGREFVSAIDSDGTT |
| | RYADSVKGRFTISQGTAKNTVYLQMNSLQPEDTAMYYCKTVCVVGSRWSDYWGQGTQV |
| | TVS |
| 59 | QVQLQESGGGLVQPGGSLRLSCAASGFSFSSYAMKWVRQAPGKGLEWVSTISSGGSSTNY |
| | ADSVKGRFTISRDNAKNTLYLQLNSLKIEDTAMYYCAKAIVPTGATMERGQGTQVTVSSG |
| | GGSQVQLQESGGGSVQAGGSLTLSCAASEYAYSTCNMGWYRQAPGKERELVSAFISDGST |
| | YYADSVKGRFTITRDNAKNTVYLQMNSLKPEDTAIYYCSANCYRRLRNYWGQGTQVTVS |
| 60 | QVQLQESGGGLVQPGGSLRLSCAASGFSFSSYAMKWVRQAPGKGLEWVSTISSGGSSTNY |
| | ADSVKGRFTISRDNAKNTLYLQLNSLKIEDTAMYYCAKAIVPTGATMERGQGTQVTVSSG |
| | GGSQVQLQESGGGLVQPGGSLRLSCTASGLTFDDSVMGWFRQAPGKGREAVSCISSSGAN |
| | AFYADSVKGRFTISRDNAKNTLYLQMNSLKPEDTATYYCKRGHACAGYYPIPYDDYWGQ |
| | GTQVTVS |
| 61 | QVQLQESGGGSVQSGGSLRLSCAASGFTYSTSNSWMAWFRQAPGKEREGVAAIYTVGGSIF |
| | YADSVRGRFTISQDATKNMFYLQMNTLKPEDTAMYYCAAASGRLRGKWFWPYEYNYWG |
| | QGTQVTVSSGGGSQVQLQESGGGSVQAGGSLRLSCTASGAIASGYIDSRWCMAWFRQAPG |
| | KEREGVAAIWPGGGLTVYADSVKGRFTISRDHAKNTLYLQMNNLKPEDTAMYYCAAGSP |
| | RMCPSLEFGFDYWGQGTQVTVS |
| 62 | QVQLQESGGGSVQSGGSLRLSCAASGFTYSTSNSWMAWFRQAPGKEREGVAAIYTVGGSIF |
| | YADSVRGRFTISQDATKNMFYLQMNTLKPEDTAMYYCAAASGRLRGKWFWPYEYNYWG |
| | QGTQVTVSSGGGSQVQLQESGGGSVQAGGSLRLSCTAPGFTSNSCGMDWYRQAPGKEREF |
| | VSSISTDGTTGYADSVKGRFTISKDKAKDTVYLQMNSLKPEDIGMYSCKTKDGTIATMELC |
| | DFGYWGQGTQVTVS |
| 63 | QVQLQESGGGSVQSGGSLRLSCAASGFTYSTSNSWMAWFRQAPGKEREGVAAIYTVGGSIF |
| | YADSVRGRFTISQDATKNMFYLQMNTLKPEDTAMYYCAAASGRLRGKWFWPYEYNYWG |
| | QGTQVTVSSGGGSQVQLQESGGGSVQAGGSLRLSCAASGYPYSNGYMGWFRQAPGKERE |
| | GVATIYTGDGRTYYADSVKGRFTISRDNAKNTVDLQMSSLKPEDTAMYYCAARAAPLYSS |
| | GSPLTRARYNVWGQGTQVTVS |
| 64 | QVQLQESGGGSVQSGGSLRLSCAASGFTYSTSNSWMAWFRQAPGKEREGVAAIYTVGGSIF |
| | YADSVRGRFTISQDATKNMFYLQMNTLKPEDTAMYYCAAASGRLRGKWFWPYEYNYWG |
| | QGTQVTVSSGGGSQVQLQESGGGSVQAGGSLRLSCVASASTYCTYDMHWYRQAPGKGRE |
| | FVSAIDSDGITRYADSVKGRFTISQGTAKNTVYLQMNSLQPEDTAMYYCKTVCVVGSRWS |
| | DYWGQGTQVIVS |
| 65 | QVQLQESGGGSVQSGGSLRLSCAASGFTYSTSNSWMAWFRQAPGKEREGVAAIYTVGGSIF |
| | YADSVRGRFTISQDATKNMFYLQMNTLKPEDTAMYYCAAASGRLRGKWFWPYEYNYWG |
| | QGTQVTVSSGGGSQVQLQESGGGSVQAGGSLTLSCAASEYAYSTQNMGWYRQAPGKERE |
| | LVSAFISDGSTYYADSVKGRFTITRDNAKNTVYLQMNSLKPEDTAIYYCSANCYRRLRNYW |
| | GQGTQVTVS |
| 66 | QVQLQESGGGSVQSGGSLRLSCAASGFTYSTSNSWMAWFRQAPGKEREGVAAIYTVGGSIF |
| | YADSVRGRFTISQDATKNMFYLQMNTLKPEDTAMYYCAAASGRLRGKWFWPYEYNYWG |
| | QGTQVTVSSGGGSQVQLQESGGGLVQPGGSLRLSCTASGLTFDDSVMGWFRQAPGKGRE |
| | AVSCISSSGANAFYADSVKGRFTISRDNAKNTLYLQMNSLKPEDTATYYCKRGHACAGYY |
| | PIPYDDYWGQGTQVTVS |
| 67 | QVQLQESGGGSVQAGGSLRLSCVASGYVSCDYFLPSWYRQAPGKEREFVSIIDGIGSTSYA |
| | ASVKGRFTASQDKGKNIAYLQMNTLKPEDTAMYYCKASCVRGRAISEYWGQGTQVTVSS |
| | GGGSQVQLQESGGGSVQAGGSLRLSCTASGAIASGYIDSRWCMAWFRQAPGKEREGVAAI |
| | WPGGGLTVYADSVKGRFTISRDHAKNTLYLQMNNLKPEDTAMYYCAAGSPRMCPSLEFGF |
| | DYWGQGTQVTVS |
| 68 | QVQLQESGGGSVQAGGSLRLSCVASGYVSCDYFLPSWYRQAPGKEREFVSIIDGIGSTSYA |
| | ASVKGRFTASQDKGKNIAYLQMNTLKPEDTAMYYCKASCVRGRAISEYWGQGTQVTVSS |
| | GGGSQVQLQESGGGSVQAGGSLRLSCTAPGFTSNSCGMDWYRQAPGKEREFVSSISTDGT |
| | TGYADSVKGRFTISKDKAKDTVYLQMNSLKPEDTGMYSCKTKDGTIATMELCDFGYWGQ |
| | GTQVTVS |
| 69 | QVQLQESGGGSVQAGGSLRLSCVASGYVSCDYFLPSWYRQAPGKEREFVSIIDGIGSTSYA |
| | ASVKGRFTASQDKGKNIAYLQMNTLKPEDTAMYYCKASCVRGRAISEYWGQGTQVTVSS |
| | GGGSQVQLQESGGGSVQAGGSLRLSCAASGYPYSNGYMGWFRQAPGKEREGVATIYTGD |
| | GRTYYADSVKGRFTISRDNAKNTVDLQMSSLKPEDTAMYYCAARAAPLYSSGSPLTRARY |
| | NVWGQGTQVTVS |
| 70 | QVQLQESGGGSVQAGGSLRLSCVASGYVSCDYFLPSWYRQAPGKEREFVSIIDGTGSTSYA |
| | ASVKGRFTASQDKGKNIAYLQMNTLKPEDTAMYYCKASCVRGRAISEYWGQGTQVTVSS |
| | GGGSQVQLQESGGGSVQAGGSLRLSCVASASTYCTYDMHWYRQAPGKGREFVSAIDSDG |
| | TTRYADSVKGRFTISQGTAKNTVYLQMNSLQPEDTAMYYCKTVCVVGSRWSDYWGQGTQ |
| | VTVS |
| 71 | QVQLQESGGGSVQAGGSLRLSCVASGYVSCDYFLPSWYRQAPGKEREFVSIIDGTGSTSYA |
| | ASVKGRFTASQDKGKNIAYLQMNTLKPEDTAMYYCKASCVRGRAISEYWGQGTQVTVSS |
| | GGGSQVQLQESGGGSVQAGGSLTLSCAASEYAYSTCNMGWYRQAPGKERELVSAFISDGS |
| | TYYADSVKGRFTITRDNAKNTVYLQMNSLKPEDTAIYYCSANCYRRLRNYWGQGTQVTVS |
| 72 | QVQLQESGGGSVQAGGSLRLSCVASGYVSCDYELPSWYRQAPGKEREFVSIIDGTGSTSYA |
| | ASVKGRFTASQDKGKNIAYLQMNTLKPEDTAMYYCKASCVRGRAISEYWGQGTQVTVSS |
| | GGGSQVQLQESGGGLVQPGGSLRLSCTASGLTFDDSVMGWFRQAPGKGREAVSCISSSGA |
| | NAFYADSVKGRFTISRDNAKNTLYLQMNSLKPEDTATYYCKRGHACAGYYPIPYDDYWG |
| | QGTQVTVS |
| 73 | QVQLQESGGGSVQAGGSLRLSCRASGSTYSNYCLGWFRQITGKEREGVAVINWVGGMLYF |
| | ADSVKGRFTVSQDQAKNTVYLQMNSLKPEDTAMYYCAAESVSSFSCGGWLTRPDRVPYW |
| | GQGTQVTVSSGGGSQVQLQESGGGSVQAGGSLRLSCTASGAIASGYIDSRWCMAWFRQAP |
| | GKEREGVAAIWPGGGLTVYADSVKGRFTISRDHAKNTLYLQMNNLKPEDTAMYYCAAGS |
| | PRMCPSLEFGFDYWGQGTQVTVS |
| 74 | QVQLQESGGGSVQAGGSLRLSCRASGSTYSNYCLGWFRQITGKEREGVAVINWVGGMLYF |
| | ADSVKGRFTVSQDQAKNTVYLQMNSLKPEDTAMYYCAAESVSSFSCGGWLTRPDRVPYW |
| | GQGTQVTVSSGGGSQVQLQESGGGSVQAGGSLRLSCTAPGFTSNSCGMDWYRQAPGKER |
| | EFVSSISTDGTTGYADSVKGRFTISKDKAKDTVYLQMNSLKPEDTGMYSCKTKDGTIATME |
| | LCDFGYWGQGTQVTVS |
| 75 | QVQLQESGGGSVQAGGSLRLSCRASGSTYSNYCLGWFRQITGKEREGVAVINWVGGMLYF |
| | ADSVKGRFTVSQDQAKNTVYLQMNSLKPEDTAMYYCAAESVSSFSCGGWLTRPDRVPYW |
| | GQGTQVTVSSGGGSQVQLQESGGGSVQAGGSLRLSCAASGYPYSNGYMGWFRQAPGKER |
| | EGVATIYTGDGRTYYADSVKGRFTISRDNAKNTVDLQMSSLKPEDTAMYYCAARAAPLYS |
| | SGSPLTRARYNVWGQGTQVTVS |
| 76 | QVQLQESGGGSVQAGGSLRLSCRASGSTYSNYCLGWFRQITGKEREGVAVINWVGGMLYF |
| | ADSVKGRFTVSQDQAKNTVYLQMNSLKPEDTAMYYCAAESVSSFSCGGWLTRPDRVPYW |
| | GQGTQVTVSSGGGSQVQLQESGGGSVQAGGSLRLSCVASASTYCTYDMHWYRQAPGKGR |
| | EFVSAIDSDGTTRYADSVKGRFTISQGTAKNTVYLQMNSLQPEDTAMYYCKTVCVVGSRW |
| | SDYWGQGTQVTVS |
| 77 | QVQLQESGGGSVQAGGSLRLSCRASGSTYSNYCLGWFRQITGKEREGVAVINWVGGMLYF |
| | ADSVKGRFTVSQDQAKNTVYLQMNSLKPEDTAMYYCAAESVSSFSCGGWLTRPDRVPYW |
| | GQGTQVTVSSGGGSQVQLQESGGGSVQAGGSLTLSCAASEYAYSTCNMGWYRQAPGKER |
| | ELVSAFISDGSTYYADSVKGRFTITRDNAKNTVYLQMNSLKPEDTAIYYCSANCYRRLRNY |
| | WGQGTQVTVS |
| 78 | QVQLQESGGGSVQAGGSLRLSCRASGSTYSNYCLGWFRQITGKEREGVAVINWVGGMLYF |
| | ADSVKGRFTVSQDQAKNTVYLQMNSLKPEDTAMYYCAAESVSSFSCGGWLTRPDRVPYW |
| | GQGTQVTVSSGGGSQVQLQESGGGLVQPGGSLRLSCTASGLTFDDSVMGWFRQAPGKGR |
| | EAVSCISSSGANAFYADSVKGRFTISRDNAKNTLYLQMNSLKPEDTATYYCKRGHACAGY |
| | YPIPYDDYWGQGTQVTVS |
| 79 | QVQLQESGGGLVQPGGSLRLSCAASGFTFSLSSMSWVRQAPGKGLEWVSAISSGGASTYYT |
| | DSVKGRFTISRDNAKNMLYLQLNSLKTEDTAMYYCAKGGSGYGDASRMTSPGSQGTQVT |
| | VSSGGGSQVQLQESGGGSVQAGGSLRLSCTASGAIASGYIDSRWCMAWFRQAPGKEREGV |
| | AAIWPGGGLTVYADSVKGRFTISRDHAKNTLYLQMNNLKPEDTAMYYCAAGSPRMCPSLE |
| | FGFDYWGQGTQVTVS |
| 80 | QVQLQESGGGLVQPGGSLRLSCAASGFTFSLSSMSWVRQAPGKGLEWVSAISSGGASTYYT |
| | DSVKGRFTISRDNAKNMLYLQLNSLKTEDTAMYYCAKGGSGYGDASRMTSPGSQGTQVT |
| | VSSGGGSQVQLQESGGGSVQAGGSLRLSCTAPGFTSNSCGMDWYRQAPGKEREFVSSISTD |
| | GTTGYADSVKGRFTISKDKAKDTVYLQMNSLKPEDIGMYSCKTKDGTIATMELCDFGYW |
| | GQGTQVTVS |
| 81 | QVQLQESGGGLVQPGGSLRLSCAASGFTFSLSSMSWVRQAPGKGLEWVSAISSGGASTYYT |
| | DSVKGRFTISRDNAKNMLYLQLNSLKTEDTAMYYCAKGGSGYGDASRMTSPGSQGTQVT |
| | VSSGGGSQVQLQESGGGSVQAGGSLRLSCAASGYPYSNGYMGWFRQAPGKEREGVATIY |
| | TGDGRTYYADSVKGRFTISRDNAKNTVDLQMSSLKPEDTAMYYCAARAAPLYSSGSPLTR |
| | ARYNVWGQGTQVTVS |
| 82 | QVQLQESGGGLVQPGGSLRLSCAASGFTFSLSSMSWVRQAPGKGLEWVSAISSGGASTYYT |
| | DSVKGRFTISRDNAKNMLYLQLNSLKTEDTAMYYCAKGGSGYGDASRMTSPGSQGTQVT |
| | VSSGGGSQVQLQESGGGSVQAGGSLRLSCVASASTYCTYDMHWYRQAPGKGREFVSAIDS |
| | DGTTRYADSVKGRFTISQGTAKNTVYLQMNSLQPEDTAMYYCKTVCVVGSRWSDYWGQ |
| | GTQVTVS |
| 83 | QVQLQESGGGLVQPGGSLRLSCAASGFTFSLSSMSWVRQAPGKGLEWVSAISSGGASTYYT |
| | DSVKGRFTISRDNAKNMLYLQLNSLKTEDTAMYYCAKGGSGYGDASRMTSPGSQGTQVT |
| | VSSGGGSQVQLQESGGGSVQAGGSLILSCAASEYAYSTQNMGWYRQAPGKERELVSAFIS |
| | DGSTYYADSVKGRFTITRDNAKNTVYLQMNSLKPEDTAIYYCSANCYRRLRNYWGQGTQ |
| | VTVS |
| 84 | QVQLQESGGGLVQPGGSLRLSCAASGFTFSLSSMSWVRQAPGKGLEWVSAISSGGASTYYT |
| | DSVKGRFTISRDNAKNMLYLQLNSLKTEDTAMYYCAKGGSGYGDASRMTSPGSQGTQVT |
| | VSSGGGSQVQLQESGGGLVQPGGSLRLSCTASGLTFDDSVMGWFRQAPGKGREAVSCISSS |
| | GANAFYADSVKGRFTISRDNAKNTLYLQMNSLKPEDTATYYCKRGHACAGYYPIPYDDY |
| | WGQGTQVTVS |

In some embodiments, an IL27R binding protein described herein (e.g., an IL27R binding protein comprising a sequence of any one of SEQ ID NOS: 43-84) is encoded by an isolated nucleic acid that is substantially identical to a sequence of any one of SEQ ID NOS: 151-192, as listed in Table 3C below. In some embodiments, an IL27R binding protein described herein (e.g., an IL27R binding protein comprising a sequence of any one of SEQ ID NOS: 43-84) is encoded by an isolated nucleic acid comprising a sequence having at least 90% (e.g., 91%, 92%, 93%, 94%, 95%, 96%, 97%, 98%, 99%, or 100%) sequence identity to a sequence of any one of SEQ ID NOS: 151-192, as listed in Table 3C below.

TABLE 3C

| SEQ | |
|---|---|
| ID | |
| NO | Sequence |
| 151 | CAGGTGCAGCTGCAGGAGAGCGGCGGCGGCCTGGTGCAGCCCGGCGGCAGCCTGAGGC |
| | TGAGCTGCGCCGCCAGCGGCTTCACCTTCAGCAGCTACCCCATGAGCTGGGTGAGGCA |
| | GGCCCCCGGCAAGGGCCTGGAGTGGATCAGCACCATCAGCGCCGGCGGCGACACCACC |
| | CTGTACGCCGACAGCGTGAAGGGCAGGTTCACCAGCAGCAGGGACAACGCCAAGAAC |
| | ACCCTGTACCTGCAGCTGAACAGCCTGAAGACCGAGGACGCCGCCATCTACTACTGCG |
| | CCAAGAGGATCGACTGCAACAGCGGCTACTGCTACAGGAGGAACTACTGGGGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGCGGCAGCCTGAGGCTGAGCTGCACCGCCAGCGGCGCCA |
| | TCGCCAGCGGCTACATCGACAGCAGGTGGTGCATGGCCTGGTTCAGGCAGGCCCCCGG |
| | CAAGGAGAGGGAGGGCGTGGCCGCCATCTGGCCCGGCGGCGGCCTGACCGTGTACGCC |
| | GACAGCGTGAAGGGCAGGTTCACCATCAGCAGGGACCACGCCAAGAACACCCTGTACC |
| | TGCAGATGAACAACCTGAAGCCCGAGGACACCGCCATGTACTACTGCGCCGCCGGCAG |
| | CCCCAGGATGTGCCCCAGCCTGGAGTTCGGCTTCGACTACTGGGGCCAGGGCACCCAG |
| | GTGACCGTGAGCGCTAGCCACCACCACCACCACCACCACCAC |
| 152 | CAGGTGCAGCTGCAGGAGAGCGGCGGGGGCCTGGTGCAGCCCGGCGGCAGCCTGAGGC |
| | TGAGCTGCGCCGCCAGCGGCTTCACCTTCAGCAGCTACCCCATGAGCTGGGTGAGGCA |
| | GGCCCCCGGCAAGGGCCTGGAGTGGATCAGCACCATCAGCGCCGGGGGCGACACCACC |
| | CTGTACGCCGACAGCGTGAAGGGCAGGTTCACCAGCAGCAGGGACAACGCCAAGAAC |
| | ACCCTGTACCTGCAGCTGAACAGCCTGAAGACCGAGGACGCCGCCATCTACTACTGCG |
| | CCAAGAGGATCGACTGCAACAGCGGCTACTGCTACAGGAGGAACTACTGGGGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGCGGCAGCCTGAGGCTGAGCTGCACCGCCCCCGGCTTCA |
| | CCAGCAACAGCTGCGGCATGGACTGGTACAGGCAGGCCCCCGGCAAGGAGAGGGAGTT |
| | CGTGAGCAGCATCAGCACCGACGGCACCACCGGCTACGCCGACAGCGTGAAGGGCAGG |
| | TTCACCATCAGCAAGGACAAGGCCAAGGACACCGTGTACCTGCAGATGAACAGCCTGA |
| | AGCCCGAGGACACCGGCATGTACAGCTGCAAGACCAAGGACGGCACCATCGCCACCAT |
| | GGAGCTGTGCGACTTCGGCTACTGGGGCCAGGGCACCCAGGTGACCGTGAGCGCTAGC |
| | CACCACCACCACCACCACCACCAC |
| 153 | CAGGTGCAGCTGCAGGAGAGGGGGGCGGCCTGGTGCAGCCCGGCGGCAGCCTGAGGC |
| | TGAGCTGCGCCGCCAGCGGCTTCACCTTCAGCAGCTACCCCATGAGCTGGGTGAGGCA |
| | GGCCCCCGGCAAGGGCCTGGAGTGGATCAGCACCATCAGCGCCGGCGGCGACACCACC |
| | CTGTACGCCGACAGCGTGAAGGGCAGGTTCACCAGCAGCAGGGACAACGCCAAGAAC |
| | ACCCTGTACCTGCAGCTGAACAGCCTGAAGACCGAGGACGCCGCCATCTACTACTGCG |
| | CCAAGAGGATCGACTGCAACAGCGGCTACTGCTACAGGAGGAACTACTGGGGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGGGCAGCCTGAGGCTGAGCTGCGCCGCCAGCGGCTACC |
| | CCTACAGCAACGGCTACATGGGCTGGTTCAGGCAGGCCCCCGGCAAGGAGAGGGAGGG |
| | CGTGGCCACCATCTACACCGGCGACGGCAGGACCTACTACGCCGACAGCGTGAAGGGC |
| | AGGTTCACCATCAGCAGGGACAACGCCAAGAACACCGTGGACCTGCAGATGAGCAGCC |
| | TGAAGCCCGAGGACACCGCCATGTACTACTGCGCCGCCAGGGCCGCCCCCCTGTACAG |
| | CAGCGGCAGCCCCCTGACCAGGGCCAGGTACAACGTGTGGGGCCAGGGCACCCAGGTG |
| | ACCGTGAGCGCTAGCCACCACCACCACCACCACCACCAC |
| 154 | CAGGTGCAGCTGCAGGAGAGCGGGGGGGGCCTGGTGCAGCCCGGCGGCAGCCTGAGGC |
| | TGAGCTGCGCCGCCAGCGGCTTCACCTTCAGCAGCTACCCCATGAGCTGGGTGAGGCA |
| | GGCCCCCGGCAAGGGCCTGGAGTGGATCAGCACCATCAGCGCCGGCGGCGACACCACC |
| | CTGTACGCCGACAGCGTGAAGGGCAGGTTCACCAGCAGCAGGGACAACGCCAAGAAC |
| | ACCCTGTACCTGCAGCTGAACAGCCTGAAGACCGAGGACGCCGCCATCTACTACTGGG |
| | CCAAGAGGATCGACTGCAACAGCGGCTACTGCTACAGGAGGAACTACTGGGGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGCGGCAGCCTGAGGCTGAGCTGCGTGGCCAGCGCCAGCA |
| | CCTACTGCACCTACGACATGCACTGGTACAGGCAGGCCCCCGGCAAGGGCAGGGAGTT |
| | CGTGAGCGCCATCGACAGCGACGGCACCACCAGGTACGCCGACAGCGTGAAGGGCAG |
| | GTTCACCATCAGCCAGGGCACCGCCAAGAACACCGTGTACCTGCAGATGAACAGCCTG |
| | CAGCCCGAGGACACCGCCATGTACTACTGCAAGACCGTGTGCGTGGTGGGCAGCAGGT |
| | GGAGCGACTACTGGGGCCAGGGCACCCAGGTGACCGTGAGCGCTAGCCACCACCACCA |
| | CCACCACCACCAC |
| 155 | CAGGTGCAGCTGCAGGAGAGCGGCGGCGGCCTGGTGCAGCCCGGCGGCAGCCTGAGGC |
| | TGAGCTGCGCCGCCAGCGGCTTCACCTTCAGCAGCTACCCCATGAGCTGGGTGAGGCA |
| | GGCCCCCGGCAAGGGCCTGGAGTGGATCAGCACCATCAGCGCCGGCGGCGACACCACC |
| | CTGTACGCCGACAGCGTGAAGGGCAGGTTCACCAGCAGCAGGGACAACGCCAAGAAC |
| | ACCCTGTACCTGCAGCTGAACAGCCTGAAGACCGAGGACGCCGCCATCTACTACTGCG |
| | CCAAGAGGATCGACTGCAACAGCGGCTACTGCTACAGGAGGAACTACTGGGGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGCGGCAGCCTGACCCTGAGCTGCGCCGCCAGCGAGTACG |
| | CCTACAGCACCTGCAACATGGGCTGGTACAGGCAGGCCCCCGGCAAGGAGAGGGAGCT |
| | GGTGAGCGCCTTCATCAGCGACGGCAGCACCTACTACGCCGACAGCGTGAAGGGCAGG |
| | TTCACCATCACCAGGGACAACGCCAAGAACACCGTGTACCTGCAGATGAACAGCCTGA |
| | AGCCCGAGGACACCGCCATCTACTACTGCAGCGCCAACTGCTACAGGAGGCTGAGGAA |
| | CTACTGGGGCCAGGGCACCCAGGTGACCGTGAGCGCTAGCCACCACCACCACCACCAC |
| | CACCAC |
| 156 | CAGGTGCAGCTGCAGGAGAGCGGCGGGGGCCTGGTGCAGCCCGGCGGCAGCCTGAGGC |
| | TGAGCTGCGCCGCCAGCGGCTTCACCTTCAGCAGCTACCCCATGAGCTGGGTGAGGCA |
| | GGCCCCCGGCAAGGGCCTGGAGTGGATCAGCACCATCAGCGCCGGCGGCGACACCACC |
| | CTGTACGCCGACAGCGTGAAGGGCAGGTTCACCAGCAGCAGGGACAACGCCAAGAAC |
| | ACCCTGTACCTGCAGCTGAACAGCCTGAAGACCGAGGACGCCGCCATCTACTACTGCG |
| | CCAAGAGGATCGACTGCAACAGCGGCTACTGCTACAGGAGGAACTACTGGGGCCAGGG |
| | CACCCAGGTGACCGTCTCGAGTGGGGCGGATCCCAGGTGCAGCTGCAGGAGAGCGGC |
| | GGCGGCCTGGTGCAGCCCGGGGGCAGCCTGAGGCTGAGCTGCACCGCCAGCGGCCTGA |
| | CCTTCGACGACAGCGTGATGGGCTGGTTCAGGCAGGCCCCCGGCAAGGGCAGGGAGGC |
| | CGTGAGCTGCATCAGCAGCAGCGGCGCCAACGCCTTCTACGCCGACAGCGTGAAGGGC |
| | AGGTTCACCATCAGCAGGGACAACGCCAAGAACACCCTGTACCTGCAGATGAACAGCC |
| | TGAAGCCCGAGGACACCGCCACCTACTACTGCAAGAGGGGCCACGCCTGCGCCGGCTA |
| | CTACCCCATCCCCTACGACGACTACTGGGGCCAGGGCACCCAGGTGACCGTGAGCGCT |
| | AGCCACCACCACCACCACCACCACCAC |
| 157 | CAGGTGCAGCTGCAGGAGAGCGGCGGCGGCCTGGTGCAGCCCGGCGAGAGCCTGAGG |
| | CTGAGCTGCACCGCCAGCGGCTTCACCTTCAGCAACTACGCCATGAGCTGGGTGAGGC |
| | AGGCCCCCGGCAAGGGCCTGGAGTGGGTGAGCGGCATCAACGTGGCCTACGGCATCAC |
| | CAGCTACGCCGACAGCGTGAAGGGCAGGTTCACCATCAGCAGGGACAACACCAAGAAC |
| | ACCCTGTACCTGCAGCTGAACAGCCTGAAGACCGAGGACACCGCCATCTACTACTGCGT |
| | GAAGCACAGCGGCACCACCATCCCCAGGGGCTTCATCAGCTACACCAAGAGGGGCCAG |
| | GGCACCCAGGTGACCGTCTCGAGTGGCGGCGGATCCCAGGTGCAGCTGCAGGAGAGCG |
| | GCGGCGGCAGCGTGCAGGCCGGCGGCAGCCTGAGGCTGAGCTGCACCGCCAGCGGCGC |
| | CATCGCCAGCGGCTACATCGACAGCAGGTGGTGCATGGCCTGGTTCAGGCAGGCCCCC |
| | GGCAAGGAGAGGGAGGGCGTGGCCGCCATCTGGCCCGGCGGCGGCCTGACCGTGTACG |
| | CCGACAGCGTGAAGGGCAGGTTCACCATCAGCAGGGACCACGCCAAGAACACCCTGTA |
| | CCTGCAGATGAACAACCTGAAGCCCGAGGACACCGCCATGTACTACTGCGCCGCCGGC |
| | AGCCCCAGGATGTGCCCCAGCCTGGAGTTCGGCTTCGACTACTGGGGCCAGGGCACCC |
| | AGGTGACCGTGAGCGCTAGCCACCACCACCACCACCACCACCAC |
| 158 | CAGGTGCAGCTGCAGGAGAGCGGCGGCGGCCTGGTGCAGCCCGGCGAGAGCCTGAGG |
| | CTGAGCTGCACCGCCAGCGGCTTCACCTTCAGCAACTACGCCATGAGCTGGGTGAGGC |
| | AGGCCCCCGGCAAGGGCCTGGAGTGGGTGAGCGGCATCAACGTGGCCTACGGCATCAC |
| | CAGCTACGCCGACAGCGTGAAGGGCAGGTTCACCATCAGCAGGGACAACACCAAGAAC |
| | ACCCTGTACCTGCAGCTGAACAGCCTGAAGACCGAGGACACCGCCATCTACTACTGCGT |
| | GAAGCACAGCGGCACCACCATCCCCAGGGGCTTCATCAGCTACACCAAGAGGGGCCAG |
| | GGCACCCAGGTGACCGTCTCGAGTGGCGGCGGATCCCAGGTGCAGCTGCAGGAGAGCG |
| | GCGGCGGCAGCGTGCAGGCCGGCGGCAGCCTGAGGCTGAGCTGCACCGCCCCCGGCTT |
| | CACCAGCAACAGCTGCGGCATGGACTGGTACAGGCAGGCCCCCGGCAAGGAGAGGGA |
| | GTTCGTGAGCAGCATCAGCACCGACGGCACCACCGGCTACGCCGACAGCGTGAAGGGC |
| | AGGTTCACCATCAGCAAGGACAAGGCCAAGGACACCGTGTACCTGCAGATGAACAGCC |
| | TGAAGCCCGAGGACACCGGCATGTACAGCTGCAAGACCAAGGACGGCACCATCGCCAC |
| | CATGGAGCTGTGCGACTTCGGCTACTGGGGCCAGGGCACCCAGGTGACCGTGAGCGCT |
| | AGCCACCACCACCACCACCACCACCAC |
| 159 | CAGGTGCAGCTGCAGGAGAGCGGCGGGGGCCTGGTGCAGCCCGGCGAGAGCCTGAGG |
| | CTGAGCTGCACCGCCAGCGGCTTCACCTTCAGCAACTACGCCATGAGCTGGGTGAGGC |
| | AGGCCCCCGGCAAGGGCCTGGAGTGGGTGAGCGGCATCAACGTGGCCTACGGCATCAC |
| | CAGCTACGCCGACAGCGTGAAGGGCAGGTTCACCATCAGCAGGGACAACACCAAGAAC |
| | ACCCTGTACCTGCAGCTGAACAGCCTGAAGACCGAGGACACCGCCATCTACTACTGCGT |
| | GAAGCACAGCGGCACCACCATCCCCAGGGGCTTCATCAGCTACACCAAGAGGGGCCAG |
| | GGCACCCAGGTGACCGTCTCGAGTGGCGGCGGATCCCAGGTGCAGCTGCAGGAGAGCG |
| | GCGGCGGCAGCGTGCAGGCCGGCGGCAGCCTGAGGCTGAGCTGCGCCGCCAGCGGCTA |
| | CCCCTACAGCAACGGCTACATGGGCTGGTTCAGGCAGGCCCCCGGCAAGGAGAGGGAG |
| | GGCGTGGCCACCATCTACACCGGCGACGGCAGGACCTACTACGCCGACAGCGTGAAGG |
| | GCAGGTTCACCATCAGCAGGGACAACGCCAAGAACACCGTGGACCTGCAGATGAGCAG |
| | CCTGAAGCCCGAGGACACCGCCATGTACTACTGCGCCGCCAGGGCCGCCCCCCTGTAC |
| | AGCAGCGGCAGCCCCCTGACCAGGGCCAGGTACAACGTGTGGGGCCAGGGCACCCAGG |
| | TGACCGTGAGCGCTAGCCACCACCACCACCACCACCACCAC |
| 160 | CAGGTGCAGCTGCAGGAGAGGGGGGGGCCTGGTGCAGCCCGGCGAGAGCCTGAGG |
| | CTGAGCTGCACCGCCAGCGGCTTCACCTTCAGCAACTACGCCATGAGCTGGGTGAGGC |
| | AGGCCCCCGGCAAGGGCCTGGAGTGGGTGAGCGGCATCAACGTGGCCTACGGCATCAC |
| | CAGCTACGCCGACAGCGTGAAGGGCAGGTTCACCATCAGCAGGGACAACACCAAGAAC |
| | ACCCTGTACCTGCAGCTGAACAGCCTGAAGACCGAGGACACCGCCATCTACTACTGCGT |
| | GAAGCACAGCGGCACCACCATCCCCAGGGGCTTCATCAGCTACACCAAGAGGGGCCAG |
| | GGCACCCAGGTGACCGTCTCGAGTGGCGGCGGATCCCAGGTGCAGCTGCAGGAGAGCG |
| | GCGGCGGCAGCGTGCAGGCCGGCGGCAGCCTGAGGCTGAGCTGCGTGGCCAGCGCCAG |
| | CACCTACTGCACCTACGACATGCACTGGTACAGGCAGGCCCCCGGCAAGGGCAGGGAG |
| | TTCGTGAGCGCCATCGACAGCGACGGCACCACCAGGTACGCCGACAGCGTGAAGGGCA |
| | GGTTCACCATCAGCCAGGGCACCGCCAAGAACACCGTGTACCTGCAGATGAACAGCCT |
| | GCAGCCCGAGGACACCGCCATGTACTACTGCAAGACCGTGTGCGTGGTGGGCAGCAGG |
| | TGGAGCGACTACTGGGGCCAGGGCACCCAGGTGACCGTGAGCGCTAGCCACCACCACC |
| | ACCACCACCACCAC |
| 161 | CAGGTGCAGCTGCAGGAGAGCGGCGGCGGCCTGGTGCAGCCCGGCGAGAGCCTGAGG |
| | CTGAGCTGCACCGCCAGCGGCTTCACCTTCAGCAACTACGCCATGAGCTGGGTGAGGC |
| | AGGCCCCCGGCAAGGGCCTGGAGTGGGTGAGCGGCATCAACGTGGCCTACGGCATCAC |
| | CAGCTACGCCGACAGCGTGAAGGGCAGGTTCACCATCAGCAGGGACAACACCAAGAAC |
| | ACCCTGTACCTGCAGCTGAACAGCCTGAAGACCGAGGACACCGCCATCTACTACTGCGT |
| | GAAGCACAGCGGCACCACCATCCCCAGGGGCTTCATCAGCTACACCAAGAGGGGCCAG |
| | GGCACCCAGGTGACCGTCTCGAGTGGCGGCGGATCCCAGGTGCAGCTGCAGGAGAGCG |
| | GCGGGGCAGCGTGCAGGCCGGCGGCAGCCTGACCCTGAGCTGCGCCGCCAGCGAGTA |
| | CGCCTACAGCACCTGCAACATGGGCTGGTACAGGCAGGCCCCCGGCAAGGAGAGGGAG |
| | CTGGTGAGCGCCTTCATCAGCGACGGCAGCACCTACTACGCCGACAGCGTGAAGGGCA |
| | GGTTCACCATCACCAGGGACAACGCCAAGAACACCGTGTACCTGCAGATGAACAGCCT |
| | GAAGCCCGAGGACACCGCCATCTACTACTGCAGCGCCAACTGCTACAGGAGGCTGAGG |
| | AACTACTGGGGCCAGGGCACCCAGGTGACCGTGAGCGCTAGCCACCACCACCACCACC |
| | ACCACCAC |
| 162 | CAGGTGCAGCTGCAGGAGAGCGGCGGCGGCCTGGTGCAGCCCGGCGAGAGCCTGAGG |
| | CTGAGCTGCACCGCCAGCGGCTTCACCTTCAGCAACTACGCCATGAGCTGGGTGAGGC |
| | AGGCCCCCGGCAAGGGCCTGGAGTGGGTGAGCGGCATCAACGTGGCCTACGGCATCAC |
| | CAGCTACGCCGACAGCGTGAAGGGCAGGTTCACCATCAGCAGGGACAACACCAAGAAC |
| | ACCCTGTACCTGCAGCTGAACAGCCTGAAGACCGAGGACACCGCCATCTACTACTGCGT |
| | GAAGCACAGCGGCACCACCATCCCCAGGGGCTTCATCAGCTACACCAAGAGGGGCCAG |
| | GGCACCCAGGTGACCGTCTCGAGTGGCGGCGGATCCCAGGTGCAGCTGCAGGAGAGCG |
| | GCGGCGGCCTGGTGCAGCCCGGGGGCAGCCTGAGGCTGAGCTGCACCGCCAGCGGCCT |
| | GACCTTCGACGACAGCGTGATGGGCTGGTTCAGGCAGGCCCCCGGCAAGGGCAGGGAG |
| | GCCGTGAGCTGCATCAGCAGCAGCGGCGCCAACGCCTTCTACGCCGACAGCGTGAAGG |
| | GCAGGTTCACCATCAGCAGGGACAACGCCAAGAACACCCTGTACCTGCAGATGAACAG |
| | CCTGAAGCCCGAGGACACCGCCACCTACTACTGCAAGAGGGGCCACGCCTGCGCCGGC |
| | TACTACCCCATCCCCTACGACGACTACTGGGGCCAGGGCACCCAGGTGACCGTGAGCG |
| | CTAGCCACCACCACCACCACCACCACCAC |
| 163 | CAGGTGCAGCTGCAGGAGAGCGGCGGCGGCCTGGTGCAGCCCGGCGGCAGCCTGAGGC |
| | TGAGCTGCGCCGCCAGCGGCTTCAGCTTCAGCAGCTACGCCATGAAGTGGGTGAGGCA |
| | GGCCCCCGGCAAGGGCCTGGAGTGGGTGAGCACCATCAGCAGCGGGGGCAGCAGCACC |
| | AACTACGCCGACAGCGTGAAGGGCAGGTTCACCATCAGCAGGGACAACGCCAAGAAC |
| | ACCCTGTACCTGCAGCTGAACAGCCTGAAGATCGAGGACACCGCCATGTACTACTGCG |
| | CCAAGGCCATCGTGCCCACCGGCGCCACCATGGAGAGGGGCCAGGGCACCCAGGTGAC |
| | CGTCTCGAGTGGCGGCGGATCCCAGGTGCAGCTGCAGGAGAGCGGGGGCGGCAGCGTG |
| | CAGGCCGGCGGCAGCCTGAGGCTGAGCTGCACCGCCAGCGGCGCCATCGCCAGCGGCT |
| | ACATCGACAGCAGGTGGTGCATGGCCTGGTTCAGGCAGGCCCCCGGCAAGGAGAGGGA |
| | GGGCGTGGCCGCCATCTGGCCCGGGGGCGGCCTGACCGTGTACGCCGACAGCGTGAAG |
| | GGCAGGTTCACCATCAGCAGGGACCACGCCAAGAACACCCTGTACCTGCAGATGAACA |
| | ACCTGAAGCCCGAGGACACCGCCATGTACTACTGCGCCGCCGGCAGCCCCAGGATGTG |
| | CCCCAGCCTGGAGTTCGGCTTCGACTACTGGGGCCAGGGCACCCAGGTGACCGTGAGC |
| | GCTAGCCACCACCACCACCACCACCACCAC |
| 164 | CAGGTGCAGCTGCAGGAGAGCGGCGGCGGCCTGGTGCAGCCCGGCGGCAGCCTGAGGC |
| | TGAGCTGCGCCGCCAGCGGCTTCAGCTTCAGCAGCTACGCCATGAAGTGGGTGAGGCA |
| | GGCCCCCGGCAAGGGCCTGGAGTGGGTGAGCACCATCAGCAGCGGCGGCAGCAGCACC |
| | AACTACGCCGACAGCGTGAAGGGCAGGTTCACCATCAGCAGGGACAACGCCAAGAAC |
| | ACCCTGTACCTGCAGCTGAACAGCCTGAAGATCGAGGACACCGCCATGTACTACTGCG |
| | CCAAGGCCATCGTGCCCACCGGCGCCACCATGGAGAGGGGCCAGGGCACCCAGGTGAC |
| | CGTCTCGAGTGGCGGCGGATCCCAGGTGCAGCTGCAGGAGAGCGGCGGCGGCAGCGTG |
| | CAGGCCGGCGGCAGCCTGAGGCTGAGCTGCACCGCCCCCGGCTTCACCAGCAACAGCT |
| | GCGGCATGGACTGGTACAGGCAGGCCCCCGGCAAGGAGAGGGAGTTCGTGAGCAGCAT |
| | CAGCACCGACGGCACCACCGGCTACGCCGACAGCGTGAAGGGCAGGTTCACCATCAGC |
| | AAGGACAAGGCCAAGGACACCGTGTACCTGCAGATGAACAGCCTGAAGCCCGAGGAC |
| | ACCGGCATGTACAGCTGCAAGACCAAGGACGGCACCATCGCCACCATGGAGCTGTGCG |
| | ACTTCGGCTACTGGGGCCAGGGCACCCAGGTGACCGTGAGCGCTAGCCACCACCACCA |
| | CCACCACCACCAC |
| 165 | CAGGTGCAGCTGCAGGAGAGCGGCGGCGGCCTGGTGCAGCCCGGCGGCAGCCTGAGGC |
| | TGAGCTGCGCCGCCAGCGGCTTCAGCTTCAGCAGCTACGCCATGAAGTGGGTGAGGCA |
| | GGCCCCCGGCAAGGGCCTGGAGTGGGTGAGCACCATCAGCAGCGGGGGCAGCAGCACC |
| | AACTACGCCGACAGCGTGAAGGGCAGGTTCACCATCAGCAGGGACAACGCCAAGAAC |
| | ACCCTGTACCTGCAGCTGAACAGCCTGAAGATCGAGGACACCGCCATGTACTACTGCG |
| | CCAAGGCCATCGTGCCCACCGGCGCCACCATGGAGAGGGGCCAGGGCACCCAGGTGAC |
| | CGTCTCGAGTGGCGGCGGATCCCAGGTGCAGCTGCAGGAGAGCGGCGGCGGCAGCGTG |
| | CAGGCCGGCGGCAGCCTGAGGCTGAGCTGCGCCGCCAGCGGCTACCCCTACAGCAACG |
| | GCTACATGGGCTGGTTCAGGCAGGCCCCCGGCAAGGAGAGGGAGGGCGTGGCCACCAT |
| | CTACACCGGCGACGGCAGGACCTACTACGCCGACAGCGTGAAGGGCAGGTTCACCATC |
| | AGCAGGGACAACGCCAAGAACACCGTGGACCTGCAGATGAGCAGCCTGAAGCCCGAG |
| | GACACCGCCATGTACTACTGCGCCGCCAGGGCCGCCCCCCTGTACAGCAGCGGCAGCC |
| | CCCTGACCAGGGCCAGGTACAACGTGTGGGGCCAGGGCACCCAGGTGACCGTGAGCGC |
| | TAGCCACCACCACCACCACCACCACCAC |
| 166 | CAGGTGCAGCTGCAGGAGAGCGGCGGCGGCCTGGTGCAGCCCGGCGGCAGCCTGAGGC |
| | TGAGCTGCGCCGCCAGCGGCTTCAGCTTCAGCAGCTACGCCATGAAGTGGGTGAGGCA |
| | GGCCCCCGGCAAGGGCCTGGAGTGGGTGAGCACCATCAGCAGCGGGGGCAGCAGCACC |
| | AACTACGCCGACAGCGTGAAGGGCAGGTTCACCATCAGCAGGGACAACGCCAAGAAC |
| | ACCCTGTACCTGCAGCTGAACAGCCTGAAGATCGAGGACACCGCCATGTACTACTGCG |
| | CCAAGGCCATCGTGCCCACCGGCGCCACCATGGAGAGGGGCCAGGGCACCCAGGTGAC |
| | CGTCTCGAGTGGCGGCGGATCCCAGGTGCAGCTGCAGGAGAGCGGCGGCGGCAGCGTG |
| | CAGGCCGGCGGCAGCCTGAGGCTGAGCTGCGTGGCCAGCGCCAGCACCTACTGCACCT |
| | ACGACATGCACTGGTACAGGCAGGCCCCCGGCAAGGGCAGGGAGTTCGTGAGCGCCAT |
| | CGACAGCGACGGCACCACCAGGTACGCCGACAGCGTGAAGGGCAGGTTCACCATCAGC |
| | CAGGGCACCGCCAAGAACACCGTGTACCTGCAGATGAACAGCCTGCAGCCCGAGGACA |
| | CCGCCATGTACTACTGCAAGACCGTGTGCGTGGTGGGCAGCAGGTGGAGCGACTACTG |
| | GGGCCAGGGCACCCAGGTGACCGTGAGCGCTAGCCACCACCACCACCACCACCACCAC |
| 167 | CAGGTGCAGCTGCAGGAGAGCGGCGGCGGCCTGGTGCAGCCCGGCGGCAGCCTGAGGC |
| | TGAGCTGCGCCGCCAGCGGCTTCAGCTTCAGCAGCTACGCCATGAAGTGGGTGAGGCA |
| | GGCCCCCGGCAAGGGCCTGGAGTGGGTGAGCACCATCAGCAGCGGGGCAGCAGCACC |
| | AACTACGCCGACAGCGTGAAGGGCAGGTTCACCATCAGCAGGGACAACGCCAAGAAC |
| | ACCCTGTACCTGCAGCTGAACAGCCTGAAGATCGAGGACACCGCCATGTACTACTGCG |
| | CCAAGGCCATCGTGCCCACCGGCGCCACCATGGAGAGGGGCCAGGGCACCCAGGTGAC |
| | CGTCTCGAGTGGCGGCGGATCCCAGGTGCAGCTGCAGGAGAGCGGGGGGGGCAGCGTG |
| | CAGGCCGGCGGCAGCCTGACCCTGAGCTGCGCCGCCAGCGAGTACGCCTACAGCACCT |
| | GCAACATGGGCTGGTACAGGCAGGCCCCCGGCAAGGAGAGGGAGCTGGTGAGCGCCTT |
| | CATCAGCGACGGCAGCACCTACTACGCCGACAGCGTGAAGGGCAGGTTCACCATCACC |
| | AGGGACAACGCCAAGAACACCGTGTACCTGCAGATGAACAGCCTGAAGCCCGAGGAC |
| | ACCGCCATCTACTACTGCAGCGCCAACTGCTACAGGAGGCTGAGGAACTACTGGGGCC |
| | AGGGCACCCAGGTGACCGTGAGCGCTAGCCACCACCACCACCACCACCACCAC |
| 168 | CAGGTGCAGCTGCAGGAGAGCGGCGGCGGCCTGGTGCAGCCCGGCGGCAGCCTGAGGC |
| | TGAGCTGCGCCGCCAGCGGCTTCAGCTTCAGCAGCTACGCCATGAAGTGGGTGAGGCA |
| | GGCCCCCGGCAAGGGCCTGGAGTGGGTGAGCACCATCAGCAGCGGGGGCAGCAGCACC |
| | AACTACGCCGACAGCGTGAAGGGCAGGTTCACCATCAGCAGGGACAACGCCAAGAAC |
| | ACCCTGTACCTGCAGCTGAACAGCCTGAAGATCGAGGACACCGCCATGTACTACTGCG |
| | CCAAGGCCATCGTGCCCACCGGCGCCACCATGGAGAGGGGCCAGGGCACCCAGGTGAC |
| | CGTCTCGAGTGGCGGCGGATCCCAGGTGCAGCTGCAGGAGAGCGGCGGCGGCCTGGTG |
| | CAGCCCGGGGGCAGCCTGAGGCTGAGCTGCACCGCCAGCGGCCTGACCTTCGACGACA |
| | GCGTGATGGGCTGGTTCAGGCAGGCCCCCGGCAAGGGCAGGGAGGCCGTGAGCTGCAT |
| | CAGCAGCAGCGGCGCCAACGCCTTCTACGCCGACAGCGTGAAGGGCAGGTTCACCATC |
| | AGCAGGGACAACGCCAAGAACACCCTGTACCTGCAGATGAACAGCCTGAAGCCCGAGG |
| | ACACCGCCACCTACTACTGCAAGAGGGGCCACGCCTGCGCCGGCTACTACCCCATCCCC |
| | TACGACGACTACTGGGGCCAGGGCACCCAGGTGACCGTGAGCGCTAGCCACCACCACC |
| | ACCACCACCACCAC |
| 169 | CAGGTGCAGCTGCAGGAGAGCGGCGGCGGCAGCGTGCAGAGCGGCGGCAGCCTGAGG |
| | CTGAGCTGCGCCGCCAGCGGCTTCACCTACAGCACCAGCAACAGCTGGATGGCCTGGTT |
| | CAGGCAGGCCCCCGGCAAGGAGAGGGAGGGCGTGGCCGCCATCTACACCGTGGGCGG |
| | CAGCATCTTCTACGCCGACAGCGTGAGGGGCAGGTTCACCATCAGCCAGGACGCCACC |
| | AAGAACATGTTCTACCTGCAGATGAACACCCTGAAGCCCGAGGACACCGCCATGTACT |
| | ACTGCGCCGCCGCCAGCGGCAGGCTGAGGGGCAAGTGGTTCTGGCCCTACGAGTACAA |
| | CTACTGGGGCCAGGGCACCCAGGTGACCGTCTCGAGTGGCGGCGGATCCCAGGTGCAG |
| | CTGCAGGAGAGCGGCGGCGGCAGCGTGCAGGCCGGCGGCAGCCTGAGGCTGAGCTGC |
| | ACCGCCAGCGGCGCCATCGCCAGCGGCTACATCGACAGCAGGTGGTGCATGGCCTGGT |
| | TCAGGCAGGCCCCCGGCAAGGAGAGGGAGGGCGTGGCCGCCATCTGGCCCGGCGGCG |
| | GCCTGACCGTGTACGCCGACAGCGTGAAGGGCAGGTTCACCATCAGCAGGGACCACGC |
| | CAAGAACACCCTGTACCTGCAGATGAACAACCTGAAGCCCGAGGACACCGCCATGTAC |
| | TACTGCGCCGCCGGCAGCCCCAGGATGTGCCCCAGCCTGGAGTTCGGCTTCGACTACTG |
| | GGGCCAGGGCACCCAGGTGACCGTGAGCGCTAGCCACCACCACCACCACCACCACCAC |
| 170 | CAGGTGCAGCTGCAGGAGAGCGGGGCGGCAGCGTGCAGAGCGGCGGCAGCCTGAGG |
| | CTGAGCTGCGCCGCCAGCGGCTTCACCTACAGCACCAGCAACAGCTGGATGGCCTGGTT |
| | CAGGCAGGCCCCCGGCAAGGAGAGGGAGGGCGTGGCCGCCATCTACACCGTGGGGGG |
| | CAGCATCTTCTACGCCGACAGCGTGAGGGGCAGGTTCACCATCAGCCAGGACGCCACC |
| | AAGAACATGTTCTACCTGCAGATGAACACCCTGAAGCCCGAGGACACCGCCATGTACT |
| | ACTGCGCCGCCGCCAGCGGCAGGCTGAGGGGCAAGTGGTTCTGGCCCTACGAGTACAA |
| | CTACTGGGGCCAGGGCACCCAGGTGACCGTCTCGAGTGGCGGCGGATCCCAGGTGCAG |
| | CTGCAGGAGAGCGGCGGCGGCAGCGTGCAGGCCGGCGGCAGCCTGAGGCTGAGCTGC |
| | ACCGCCCCCGGCTTCACCAGCAACAGCTGCGGCATGGACTGGTACAGGCAGGCCCCCG |
| | GCAAGGAGAGGGAGTTCGTGAGCAGCATCAGCACCGACGGCACCACCGGCTACGCCGA |
| | CAGCGTGAAGGGCAGGTTCACCATCAGCAAGGACAAGGCCAAGGACACCGTGTACCTG |
| | CAGATGAACAGCCTGAAGCCCGAGGACACCGGCATGTACAGCTGCAAGACCAAGGAC |
| | GGCACCATCGCCACCATGGAGCTGTGCGACTTCGGCTACTGGGGCCAGGGCACCCAGG |
| | TGACCGTGAGCGCTAGCCACCACCACCACCACCACCACCAC |
| 171 | CAGGTGCAGCTGCAGGAGAGCGGCGGCGGCAGCGTGCAGAGCGGCGGCAGCCTGAGG |
| | CTGAGCTGCGCCGCCAGCGGCTTCACCTACAGCACCAGCAACAGCTGGATGGCCTGGTT |
| | CAGGCAGGCCCCCGGCAAGGAGAGGGAGGGCGTGGCCGCCATCTACACCGTGGGGGG |
| | CAGCATCTTCTACGCCGACAGCGTGAGGGGCAGGTTCACCATCAGCCAGGACGCCACC |
| | AAGAACATGTTCTACCTGCAGATGAACACCCTGAAGCCCGAGGACACCGCCATGTACT |
| | ACTGCGCCGCCGCCAGCGGCAGGCTGAGGGGCAAGTGGTTCTGGCCCTACGAGTACAA |
| | CTACTGGGGCCAGGGCACCCAGGTGACCGTCTCGAGTGGCGGGGGATCCCAGGTGCAG |
| | CTGCAGGAGAGCGGGGGGGGCAGCGTGCAGGCCGGCGGCAGCCTGAGGCTGAGCTGC |
| | GCCGCCAGCGGCTACCCCTACAGCAACGGCTACATGGGCTGGTTCAGGCAGGCCCCCG |
| | GCAAGGAGAGGGAGGGCGTGGCCACCATCTACACCGGCGACGGCAGGACCTACTACGC |
| | CGACAGCGTGAAGGGCAGGTTCACCATCAGCAGGGACAACGCCAAGAACACCGTGGA |
| | CCTGCAGATGAGCAGCCTGAAGCCCGAGGACACCGCCATGTACTACTGCGCCGCCAGG |
| | GCCGCCCCCCTGTACAGCAGCGGCAGCCCCCTGACCAGGGCCAGGTACAACGTGTGGG |
| | GCCAGGGCACCCAGGTGACCGTGAGCGCTAGCCACCACCACCACCACCACCACCAC |
| 172 | CAGGTGCAGCTGCAGGAGAGCGGCGGCGGCAGCGTGCAGAGCGGCGGCAGCCTGAGG |
| | CTGAGCTGCGCCGCCAGCGGCTTCACCTACAGCACCAGCAACAGCTGGATGGCCTGGTT |
| | CAGGCAGGCCCCCGGCAAGGAGAGGGAGGGCGTGGCCGCCATCTACACCGTGGGCGG |
| | CAGCATCTTCTACGCCGACAGCGTGAGGGGCAGGTTCACCATCAGCCAGGACGCCACC |
| | AAGAACATGTTCTACCTGCAGATGAACACCCTGAAGCCCGAGGACACCGCCATGTACT |
| | ACTGCGCCGCCGCCAGCGGCAGGCTGAGGGGCAAGTGGTTCTGGCCCTACGAGTACAA |
| | CTACTGGGGCCAGGGCACCCAGGTGACCGTCTCGAGTGGCGGCGGATCCCAGGTGCAG |
| | CTGCAGGAGAGCGGCGGCGGCAGCGTGCAGGCCGGCGGCAGCCTGAGGCTGAGCTGC |
| | GTGGCCAGCGCCAGCACCTACTGCACCTACGACATGCACTGGTACAGGCAGGCCCCCG |
| | GCAAGGGCAGGGAGTTCGTGAGCGCCATCGACAGCGACGGCACCACCAGGTACGCCGA |
| | CAGCGTGAAGGGCAGGTTCACCATCAGCCAGGGCACCGCCAAGAACACCGTGTACCTG |
| | CAGATGAACAGCCTGCAGCCCGAGGACACCGCCATGTACTACTGCAAGACCGTGTGCG |
| | TGGTGGGCAGCAGGTGGAGCGACTACTGGGGCCAGGGCACCCAGGTGACCGTGAGCGC |
| | TAGCCACCACCACCACCACCACCACCAC |
| 173 | CAGGTGCAGCTGCAGGAGAGCGGCGGCGGCAGCGTGCAGAGCGGCGGCAGCCTGAGG |
| | CTGAGCTGCGCCGCCAGCGGCTTCACCTACAGCACCAGCAACAGCTGGATGGCCTGGTT |
| | CAGGCAGGCCCCCGGCAAGGAGAGGGAGGGCGTGGCCGCCATCTACACCGTGGGGGG |
| | CAGCATCTTCTACGCCGACAGCGTGAGGGGCAGGTTCACCATCAGCCAGGACGCCACC |
| | AAGAACATGTTCTACCTGCAGATGAACACCCTGAAGCCCGAGGACACCGCCATGTACT |
| | ACTGCGCCGCCGCCAGCGGCAGGCTGAGGGGCAAGTGGTTCTGGCCCTACGAGTACAA |
| | CTACTGGGGCCAGGGCACCCAGGTGACCGTCTCGAGTGGCGGCGGATCCCAGGTGCAG |
| | CTGCAGGAGAGCGGCGGCGGCAGCGTGCAGGCCGGCGGCAGCCTGACCCTGAGCTGCG |
| | CCGCCAGCGAGTACGCCTACAGCACCTGCAACATGGGCTGGTACAGGCAGGCCCCCGG |
| | CAAGGAGAGGGAGCTGGTGAGCGCCTTCATCAGCGACGGCAGCACCTACTACGCCGAC |
| | AGCGTGAAGGGCAGGTTCACCATCACCAGGGACAACGCCAAGAACACCGTGTACCTGC |
| | AGATGAACAGCCTGAAGCCCGAGGACACCGCCATCTACTACTGCAGCGCCAACTGCTA |
| | CAGGAGGCTGAGGAACTACTGGGGCCAGGGCACCCAGGTGACCGTGAGCGCTAGCCAC |
| | CACCACCACCACCACCACCAC |
| 174 | CAGGTGCAGCTGCAGGAGAGCGGCGGCGGCAGCGTGCAGAGCGGCGGCAGCCTGAGG |
| | CTGAGCTGCGCCGCCAGCGGCTTCACCTACAGCACCAGCAACAGCTGGATGGCCTGGTT |
| | CAGGCAGGCCCCCGGCAAGGAGAGGGAGGGCGTGGCCGCCATCTACACCGTGGGGGG |
| | CAGCATCTTCTACGCCGACAGCGTGAGGGGCAGGTTCACCATCAGCCAGGACGCCACC |
| | AAGAACATGTTCTACCTGCAGATGAACACCCTGAAGCCCGAGGACACCGCCATGTACT |
| | ACTGCGCCGCCGCCAGCGGCAGGCTGAGGGGCAAGTGGTTCTGGCCCTACGAGTACAA |
| | CTACTGGGGCCAGGGCACCCAGGTGACCGTCTCGAGTGGCGGCGGATCCCAGGTGCAG |
| | CTGCAGGAGAGCGGGGGGGGCCTGGTGCAGCCCGGCGGCAGCCTGAGGCTGAGCTGCA |
| | CCGCCAGCGGCCTGACCTTCGACGACAGCGTGATGGGCTGGTTCAGGCAGGCCCCCGG |
| | CAAGGGCAGGGAGGCCGTGAGCTGCATCAGCAGCAGCGGCGCCAACGCCTTCTACGCC |
| | GACAGCGTGAAGGGCAGGTTCACCATCAGCAGGGACAACGCCAAGAACACCCTGTACC |
| | TGCAGATGAACAGCCTGAAGCCCGAGGACACCGCCACCTACTACTGCAAGAGGGGCCA |
| | CGCCTGCGCCGGCTACTACCCCATCCCCTACGACGACTACTGGGGCCAGGGCACCCAG |
| | GTGACCGTGAGCGCTAGCCACCACCACCACCACCACCACCAC |
| 175 | CAGGTGCAGCTGCAGGAGAGCGGCGGCGGCAGCGTGCAGGCCGGGGGCAGCCTGAGG |
| | CTGAGCTGCGTGGCCAGCGGCTACGTGAGCTGCGACTACTTCCTGCCCAGCTGGTACAG |
| | GCAGGCCCCCGGCAAGGAGAGGGAGTTCGTGAGCATCATCGACGGCACCGGCAGCACC |
| | AGCTACGCCGCCAGCGTGAAGGGCAGGTTCACCGCCAGCCAGGACAAGGGCAAGAAC |
| | ATCGCCTACCTGCAGATGAACACCCTGAAGCCCGAGGACACCGCCATGTACTACTGCA |
| | AGGCCAGCTGCGTGAGGGGCAGGGCCATCAGCGAGTACTGGGGCCAGGGCACCCAGGT |
| | GACCGTCTCGAGTGGCGGCGGATCCCAGGTGCAGCTGCAGGAGAGCGGGGGGGGCAGC |
| | GTGCAGGCCGGCGGCAGCCTGAGGCTGAGCTGCACCGCCAGCGGCGCCATCGCCAGCG |
| | GCTACATCGACAGCAGGTGGTGCATGGCCTGGTTCAGGCAGGCCCCCGGCAAGGAGAG |
| | GGAGGGCGTGGCCGCCATCTGGCCCGGCGGCGGCCTGACCGTGTACGCCGACAGCGTG |
| | AAGGGCAGGTTCACCATCAGCAGGGACCACGCCAAGAACACCCTGTACCTGCAGATGA |
| | ACAACCTGAAGCCCGAGGACACCGCCATGTACTACTGCGCCGCCGGCAGCCCCAGGAT |
| | GTGCCCCAGCCTGGAGTTCGGCTTCGACTACTGGGGCCAGGGCACCCAGGTGACCGTG |
| | AGCGCTAGCCACCACCACCACCACCACCACCAC |
| 176 | CAGGTGCAGCTGCAGGAGAGCGGCGGGGGCAGCGTGCAGGCCGGCGGCAGCCTGAGG |
| | CTGAGCTGCGTGGCCAGCGGCTACGTGAGCTGCGACTACTTCCTGCCCAGCTGGTACAG |
| | GCAGGCCCCCGGCAAGGAGAGGGAGTTCGTGAGCATCATCGACGGCACCGGCAGCACC |
| | AGCTACGCCGCCAGCGTGAAGGGCAGGTTCACCGCCAGCCAGGACAAGGGCAAGAAC |
| | ATCGCCTACCTGCAGATGAACACCCTGAAGCCCGAGGACACCGCCATGTACTACTGCA |
| | AGGCCAGCTGCGTGAGGGGCAGGGCCATCAGCGAGTACTGGGGCCAGGGCACCCAGGT |
| | GACCGTCTCGAGTGGCGGCGGATCCCAGGTGCAGCTGCAGGAGAGCGGCGGCGGCAGC |
| | GTGCAGGCCGGCGGCAGCCTGAGGCTGAGCTGCACCGCCCCCGGCTTCACCAGCAACA |
| | GCTGCGGCATGGACTGGTACAGGCAGGCCCCCGGCAAGGAGAGGGAGTTCGTGAGCAG |
| | CATCAGCACCGACGGCACCACCGGCTACGCCGACAGCGTGAAGGGCAGGTTCACCATC |
| | AGCAAGGACAAGGCCAAGGACACCGTGTACCTGCAGATGAACAGCCTGAAGCCCGAG |
| | GACACCGGCATGTACAGCTGCAAGACCAAGGACGGCACCATCGCCACCATGGAGCTGT |
| | GCGACTTCGGCTACTGGGGCCAGGGCACCCAGGTGACCGTGAGCGCTAGCCACCACCA |
| | CCACCACCACCACCAC |
| 177 | CAGGTGCAGCTGCAGGAGAGCGGCGGCGGCAGCGTGCAGGCCGGGGGCAGCCTGAGG |
| | CTGAGCTGCGTGGCCAGCGGCTACGTGAGCTGCGACTACTTCCTGCCCAGCTGGTACAG |
| | GCAGGCCCCCGGCAAGGAGAGGGAGTTCGTGAGCATCATCGACGGCACCGGCAGCACC |
| | AGCTACGCCGCCAGCGTGAAGGGCAGGTTCACCGCCAGCCAGGACAAGGGCAAGAAC |
| | ATCGCCTACCTGCAGATGAACACCCTGAAGCCCGAGGACACCGCCATGTACTACTGCA |
| | AGGCCAGCTGCGTGAGGGGCAGGGCCATCAGCGAGTACTGGGGCCAGGGCACCCAGGT |
| | GACCGTCTCGAGTGGCGGCGGATCCCAGGTGCAGCTGCAGGAGAGCGGGGGGGGCAGC |
| | GTGCAGGCCGGCGGCAGCCTGAGGCTGAGCTGCGCCGCCAGCGGCTACCCCTACAGCA |
| | ACGGCTACATGGGCTGGTTCAGGCAGGCCCCCGGCAAGGAGAGGGAGGGCGTGGCCAC |
| | CATCTACACCGGCGACGGCAGGACCTACTACGCCGACAGCGTGAAGGGCAGGTTCACC |
| | ATCAGCAGGGACAACGCCAAGAACACCGTGGACCTGCAGATGAGCAGCCTGAAGCCCG |
| | AGGACACCGCCATGTACTACTGCGCCGCCAGGGCCGCCCCCCTGTACAGCAGCGGCAG |
| | CCCCCTGACCAGGGCCAGGTACAACGTGTGGGGCCAGGGCACCCAGGTGACCGTGAGC |
| | GCTAGCCACCACCACCACCACCACCACCAC |
| 178 | CAGGTGCAGCTGCAGGAGAGCGGCGGCGGCAGCGTGCAGGCCGGGGGCAGCCTGAGG |
| | CTGAGCTGCGTGGCCAGCGGCTACGTGAGCTGCGACTACTTCCTGCCCAGCTGGTACAG |
| | GCAGGCCCCCGGCAAGGAGAGGGAGTTCGTGAGCATCATCGACGGCACCGGCAGCACC |
| | AGCTACGCCGCCAGCGTGAAGGGCAGGTTCACCGCCAGCCAGGACAAGGGCAAGAAC |
| | ATCGCCTACCTGCAGATGAACACCCTGAAGCCCGAGGACACCGCCATGTACTACTGCA |
| | AGGCCAGCTGCGTGAGGGGCAGGGCCATCAGCGAGTACTGGGGCCAGGGCACCCAGGT |
| | GACCGTCTCGAGTGGCGGCGGATCCCAGGTGCAGCTGCAGGAGAGCGGCGGCGGCAGC |
| | GTGCAGGCCGGCGGCAGCCTGAGGCTGAGCTGCGTGGCCAGCGCCAGCACCTACTGCA |
| | CCTACGACATGCACTGGTACAGGCAGGCCCCCGGCAAGGGCAGGGAGTTCGTGAGCGC |
| | CATCGACAGCGACGGCACCACCAGGTACGCCGACAGCGTGAAGGGCAGGTTCACCATC |
| | AGCCAGGGCACCGCCAAGAACACCGTGTACCTGCAGATGAACAGCCTGCAGCCCGAGG |
| | ACACCGCCATGTACTACTGCAAGACCGTGTGCGTGGTGGGCAGCAGGTGGAGCGACTA |
| | CTGGGGCCAGGGCACCCAGGTGACCGTGAGCGCTAGCCACCACCACCACCACCACCAC |
| | CAC |
| 179 | CAGGTGCAGCTGCAGGAGAGCGGCGGGGGCAGCGTGCAGGCCGGCGGCAGCCTGAGG |
| | CTGAGCTGCGTGGCCAGCGGCTACGTGAGCTGCGACTACTTCCTGCCCAGCTGGTACAG |
| | GCAGGCCCCCGGCAAGGAGAGGGAGTTCGTGAGCATCATCGACGGCACCGGCAGCACC |
| | AGCTACGCCGCCAGCGTGAAGGGCAGGTTCACCGCCAGCCAGGACAAGGGCAAGAAC |
| | ATCGCCTACCTGCAGATGAACACCCTGAAGCCCGAGGACACCGCCATGTACTACTGCA |
| | AGGCCAGCTGCGTGAGGGGCAGGGCCATCAGCGAGTACTGGGGCCAGGGCACCCAGGT |
| | GACCGTCTCGAGTGGCGGCGGATCCCAGGTGCAGCTGCAGGAGAGCGGCGGCGGCAGC |
| | GTGCAGGCCGGCGGCAGCCTGACCCTGAGCTGCGCCGCCAGCGAGTACGCCTACAGCA |
| | CCTGCAACATGGGCTGGTACAGGCAGGCCCCCGGCAAGGAGAGGGAGCTGGTGAGCGC |
| | CTTCATCAGCGACGGCAGCACCTACTACGCCGACAGCGTGAAGGGCAGGTTCACCATC |
| | ACCAGGGACAACGCCAAGAACACCGTGTACCTGCAGATGAACAGCCTGAAGCCCGAGG |
| | ACACCGCCATCTACTACTGCAGCGCCAACTGCTACAGGAGGCTGAGGAACTACTGGGG |
| | CCAGGGCACCCAGGTGACCGTGAGCGCTAGCCACCACCACCACCACCACCACCAC |
| 180 | CAGGTGCAGCTGCAGGAGAGCGGCGGGGGCAGCGTGCAGGCCGGCGGCAGCCTGAGG |
| | CTGAGCTGCGTGGCCAGCGGCTACGTGAGCTGCGACTACTTCCTGCCCAGCTGGTACAG |
| | GCAGGCCCCCGGCAAGGAGAGGGAGTTCGTGAGCATCATCGACGGCACCGGCAGCACC |
| | AGCTACGCCGCCAGCGTGAAGGGCAGGTTCACCGCCAGCCAGGACAAGGGCAAGAAC |
| | ATCGCCTACCTGCAGATGAACACCCTGAAGCCCGAGGACACCGCCATGTACTACTGCA |
| | AGGCCAGCTGCGTGAGGGGCAGGGCCATCAGCGAGTACTGGGGCCAGGGCACCCAGGT |
| | GACCGTCTCGAGTGGCGGCGGATCCCAGGTGCAGCTGCAGGAGAGCGGCGGCGGCCTG |
| | GTGCAGCCCGGCGGCAGCCTGAGGCTGAGCTGCACCGCCAGCGGCCTGACCTTCGACG |
| | ACAGCGTGATGGGCTGGTTCAGGCAGGCCCCCGGCAAGGGCAGGGAGGCCGTGAGCTG |
| | CATCAGCAGCAGCGGCGCCAACGCCTTCTACGCCGACAGCGTGAAGGGCAGGTTCACC |
| | ATCAGCAGGGACAACGCCAAGAACACCCTGTACCTGCAGATGAACAGCCTGAAGCCCG |
| | AGGACACCGCCACCTACTACTGCAAGAGGGGCCACGCCTGCGCCGGCTACTACCCCAT |
| | CCCCTACGACGACTACTGGGGCCAGGGCACCCAGGTGACCGTGAGCGCTAGCCACCAC |
| | CACCACCACCACCACCAC |
| 181 | CAGGTGCAGCTGCAGGAGAGCGGCGGCGGCAGCGTGCAGGCCGGGGGCAGCCTGAGG |
| | CTGAGCTGCAGGGCCAGCGGCAGCACCTACAGCAACTACTGCCTGGGCTGGTTCAGGC |
| | AGATCACCGGCAAGGAGAGGGAGGGCGTGGCCGTGATCAACTGGGGGGGGCATGCT |
| | GTACTTCGCCGACAGCGTGAAGGGCAGGTTCACCGTGAGCCAGGACCAGGCCAAGAAC |
| | ACCGTGTACCTGCAGATGAACAGCCTGAAGCCCGAGGACACCGCCATGTACTACTGCG |
| | CCGCCGAGAGCGTGAGCAGCTTCAGCTGCGGCGGCTGGCTGACCAGGCCCGACAGGGT |
| | GCCCTACTGGGGCCAGGGCACCCAGGTGACCGTCTCGAGTGGCGGCGGATCCCAGGTG |
| | CAGCTGCAGGAGAGCGGGGGGGGCAGCGTGCAGGCCGGCGGCAGCCTGAGGCTGAGC |
| | TGCACCGCCAGCGGCGCCATCGCCAGCGGCTACATCGACAGCAGGTGGTGCATGGCCT |
| | GGTTCAGGCAGGCCCCCGGCAAGGAGAGGGAGGGCGTGGCCGCCATCTGGCCCGGCGG |
| | CGGCCTGACCGTGTACGCCGACAGCGTGAAGGGCAGGTTCACCATCAGCAGGGACCAC |
| | GCCAAGAACACCCTGTACCTGCAGATGAACAACCTGAAGCCCGAGGACACCGCCATGT |
| | ACTACTGCGCCGCCGGCAGCCCCAGGATGTGCCCCAGCCTGGAGTTCGGCTTCGACTAC |
| | TGGGGCCAGGGCACCCAGGTGACCGTGAGCGCTAGCCACCACCACCACCACCACCACC |
| | AC |
| 182 | CAGGTGCAGCTGCAGGAGAGCGGCGGGGGCAGCGTGCAGGCCGGCGGCAGCCTGAGG |
| | CTGAGCTGCAGGGCCAGCGGCAGCACCTACAGCAACTACTGCCTGGGCTGGTTCAGGC |
| | AGATCACCGGCAAGGAGAGGGAGGGCGTGGCCGTGATCAACTGGGGGGGGCATGCT |
| | GTACTTCGCCGACAGCGTGAAGGGCAGGTTCACCGTGAGCCAGGACCAGGCCAAGAAC |
| | ACCGTGTACCTGCAGATGAACAGCCTGAAGCCCGAGGACACCGCCATGTACTACTGCG |
| | CCGCCGAGAGCGTGAGCAGCTTCAGCTGCGGCGGCTGGCTGACCAGGCCCGACAGGGT |
| | GCCCTACTGGGGCCAGGGCACCCAGGTGACCGTCTCGAGTGGCGGCGGATCCCAGGTG |
| | CAGCTGCAGGAGAGCGGGGGGGGCAGCGTGCAGGCCGGCGGCAGCCTGAGGCTGAGC |
| | TGCACCGCCCCCGGCTTCACCAGCAACAGCTGCGGCATGGACTGGTACAGGCAGGCCC |
| | CCGGCAAGGAGAGGGAGTTCGTGAGCAGCATCAGCACCGACGGCACCACCGGCTACGC |
| | CGACAGCGTGAAGGGCAGGTTCACCATCAGCAAGGACAAGGCCAAGGACACCGTGTAC |
| | CTGCAGATGAACAGCCTGAAGCCCGAGGACACCGGCATGTACAGCTGCAAGACCAAGG |
| | ACGGCACCATCGCCACCATGGAGCTGTGCGACTTCGGCTACTGGGGCCAGGGCACCCA |
| | GGTGACCGTGAGCGCTAGCCACCACCACCACCACCACCACCAC |
| 183 | CAGGTGCAGCTGCAGGAGAGCGGCGGCGGCAGCGTGCAGGCCGGCGGCAGCCTGAGG |
| | CTGAGCTGCAGGGCCAGCGGCAGCACCTACAGCAACTACTGCCTGGGCTGGTTCAGGC |
| | AGATCACCGGCAAGGAGAGGGAGGGCGTGGCCGTGATCAACTGGGTGGGCGGCATGCT |
| | GTACTTCGCCGACAGCGTGAAGGGCAGGTTCACCGTGAGCCAGGACCAGGCCAAGAAC |
| | ACCGTGTACCTGCAGATGAACAGCCTGAAGCCCGAGGACACCGCCATGTACTACTGCG |
| | CCGCCGAGAGCGTGAGCAGCTTCAGCTGCGGCGGCTGGCTGACCAGGCCCGACAGGGT |
| | GCCCTACTGGGGCCAGGGCACCCAGGTGACCGTCTCGAGTGGGGGGGGATCCCAGGTG |
| | CAGCTGCAGGAGAGCGGCGGCGGCAGCGTGCAGGCCGGCGGCAGCCTGAGGCTGAGC |
| | TGCGCCGCCAGCGGCTACCCCTACAGCAACGGCTACATGGGCTGGTTCAGGCAGGCCC |
| | CCGGCAAGGAGAGGGAGGGCGTGGCCACCATCTACACCGGCGACGGCAGGACCTACTA |
| | CGCCGACAGCGTGAAGGGCAGGTTCACCATCAGCAGGGACAACGCCAAGAACACCGTG |
| | GACCTGCAGATGAGCAGCCTGAAGCCCGAGGACACCGCCATGTACTACTGCGCCGCCA |
| | GGGCCGCCCCCCTGTACAGCAGCGGCAGCCCCCTGACCAGGGCCAGGTACAACGTGTG |
| | GGGCCAGGGCACCCAGGTGACCGTGAGCGCTAGCCACCACCACCACCACCACCACCAC |
| 184 | CAGGTGCAGCTGCAGGAGAGCGGCGGCGGCAGCGTGCAGGCCGGCGGCAGCCTGAGG |
| | CTGAGCTGCAGGGCCAGCGGCAGCACCTACAGCAACTACTGCCTGGGCTGGTTCAGGC |
| | AGATCACCGGCAAGGAGAGGGAGGGCGTGGCCGTGATCAACTGGGGGGCGGCATGCT |
| | GTACTTCGCCGACAGCGTGAAGGGCAGGTTCACCGTGAGCCAGGACCAGGCCAAGAAC |
| | ACCGTGTACCTGCAGATGAACAGCCTGAAGCCCGAGGACACCGCCATGTACTACTGCG |
| | CCGCCGAGAGCGTGAGCAGCTTCAGCTGCGGCGGCTGGCTGACCAGGCCCGACAGGGT |
| | GCCCTACTGGGGCCAGGGCACCCAGGTGACCGTCTCGAGTGGCGGCGGATCCCAGGTG |
| | CAGCTGCAGGAGAGCGGCGGCGGCAGCGTGCAGGCCGGCGGCAGCCTGAGGCTGAGC |
| | TGCGTGGCCAGCGCCAGCACCTACTGCACCTACGACATGCACTGGTACAGGCAGGCCC |
| | CCGGCAAGGGCAGGGAGTTCGTGAGCGCCATCGACAGCGACGGCACCACCAGGTACGC |
| | CGACAGCGTGAAGGGCAGGTTCACCATCAGCCAGGGCACCGCCAAGAACACCGTGTAC |
| | CTGCAGATGAACAGCCTGCAGCCCGAGGACACCGCCATGTACTACTGCAAGACCGTGT |
| | GCGTGGTGGGCAGCAGGTGGAGCGACTACTGGGGCCAGGGCACCCAGGTGACCGTGAG |
| | CGCTAGCCACCACCACCACCACCACCACCAC |
| 185 | CAGGTGCAGCTGCAGGAGAGCGGCGGCGGCAGCGTGCAGGCCGGCGGCAGCCTGAGG |
| | CTGAGCTGCAGGGCCAGGGGCAGCACCTACAGCAACTACTGCCTGGGCTGGTTCAGGC |
| | AGATCACCGGCAAGGAGAGGGAGGGCGTGGCCGTGATCAACTGGGTGGGCGGCATGCT |
| | GTACTTCGCCGACAGCGTGAAGGGCAGGTTCACCGTGAGCCAGGACCAGGCCAAGAAC |
| | ACCGTGTACCTGCAGATGAACAGCCTGAAGCCCGAGGACACCGCCATGTACTACTGCG |
| | CCGCCGAGAGCGTGAGCAGCTTCAGCTGCGGCGGCTGGCTGACCAGGCCCGACAGGGT |
| | GCCCTACTGGGGCCAGGGCACCCAGGTGACCGTCTCGAGTGGCGGCGGATCCCAGGTG |
| | CAGCTGCAGGAGAGCGGGGGGGGCAGCGTGCAGGCCGGCGGCAGCCTGACCCTGAGCT |
| | GCGCCGCCAGCGAGTACGCCTACAGCACCTGCAACATGGGCTGGTACAGGCAGGCCCC |
| | CGGCAAGGAGAGGGAGCTGGTGAGCGCCTTCATCAGCGACGGCAGCACCTACTACGCC |
| | GACAGCGTGAAGGGCAGGTTCACCATCACCAGGGACAACGCCAAGAACACCGTGTACC |
| | TGCAGATGAACAGCCTGAAGCCCGAGGACACCGCCATCTACTACTGCAGCGCCAACTG |
| | CTACAGGAGGCTGAGGAACTACTGGGGCCAGGGCACCCAGGTGACCGTGAGCGCTAGC |
| | CACCACCACCACCACCACCACCAC |
| 186 | CAGGTGCAGCTGCAGGAGAGCGGCGGCGGCAGCGTGCAGGCCGGCGGCAGCCTGAGG |
| | CTGAGCTGCAGGGCCAGGGGCAGCACCTACAGCAACTACTGCCTGGGCTGGTTCAGGC |
| | AGATCACCGGCAAGGAGAGGGAGGGCGTGGCCGTGATCAACTGGGTGGGCGGCATGCT |
| | GTACTTCGCCGACAGCGTGAAGGGCAGGTTCACCGTGAGCCAGGACCAGGCCAAGAAC |
| | ACCGTGTACCTGCAGATGAACAGCCTGAAGCCCGAGGACACCGCCATGTACTACTGCG |
| | CCGCCGAGAGCGTGAGCAGCTTCAGCTGCGGCGGCTGGCTGACCAGGCCCGACAGGGT |
| | GCCCTACTGGGGCCAGGGCACCCAGGTGACCGTCTCGAGTGGGGGGGGATCCCAGGTG |
| | CAGCTGCAGGAGAGCGGCGGCGGCCTGGTGCAGCCCGGCGGCAGCCTGAGGCTGAGCT |
| | GCACCGCCAGCGGCCTGACCTTCGACGACAGCGTGATGGGCTGGTTCAGGCAGGCCCC |
| | CGGCAAGGGCAGGGAGGCCGTGAGCTGCATCAGCAGCAGCGGCGCCAACGCCTTCTAC |
| | GCCGACAGCGTGAAGGGCAGGTTCACCATCAGCAGGGACAACGCCAAGAACACCCTGT |
| | ACCTGCAGATGAACAGCCTGAAGCCCGAGGACACCGCCACCTACTACTGCAAGAGGGG |
| | CCACGCCTGCGCCGGCTACTACCCCATCCCCTACGACGACTACTGGGGCCAGGGCACCC |
| | AGGTGACCGTGAGCGCTAGCCACCACCACCACCACCACCACCAC |
| 187 | CAGGTGCAGCTGCAGGAGAGCGGCGGCGGCCTGGTGCAGCCCGGCGGCAGCCTGAGGC |
| | TGAGCTGCGCCGCCAGCGGCTTCACCTTCAGCCTGAGCAGCATGAGCTGGGTGAGGCA |
| | GGCCCCCGGCAAGGGCCTGGAGTGGGTGAGCGCCATCAGCAGCGGCGGCGCCAGCACC |
| | TACTACACCGACAGCGTGAAGGGCAGGTTCACCATCAGCAGGGACAACGCCAAGAACA |
| | TGCTGTACCTGCAGCTGAACAGCCTGAAGACCGAGGACACCGCCATGTACTACTGCGC |
| | CAAGGGCGGCAGCGGCTACGGCGACGCCAGCAGGATGACCAGCCCCGGCAGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGGGGCAGCCTGAGGCTGAGCTGCACCGCCAGCGGCGCCA |
| | TCGCCAGCGGCTACATCGACAGCAGGTGGTGCATGGCCTGGTTCAGGCAGGCCCCCGG |
| | CAAGGAGAGGGAGGGCGTGGCCGCCATCTGGCCCGGGGGGGGCCTGACCGTGTACGCC |
| | GACAGCGTGAAGGGCAGGTTCACCATCAGCAGGGACCACGCCAAGAACACCCTGTACC |
| | TGCAGATGAACAACCTGAAGCCCGAGGACACCGCCATGTACTACTGCGCCGCCGGCAG |
| | CCCCAGGATGTGCCCCAGCCTGGAGTTCGGCTTCGACTACTGGGGCCAGGGCACCCAG |
| | GTGACCGTGAGCGCTAGCCACCACCACCACCACCACCACCAC |
| 188 | CAGGTGCAGCTGCAGGAGAGCGGCGGCGGCCTGGTGCAGCCCGGCGGCAGCCTGAGGC |
| | TGAGCTGCGCCGCCAGCGGCTTCACCTTCAGCCTGAGCAGCATGAGCTGGGTGAGGCA |
| | GGCCCCCGGCAAGGGCCTGGAGTGGGTGAGCGCCATCAGCAGCGGGGGCGCCAGCACC |
| | TACTACACCGACAGCGTGAAGGGCAGGTTCACCATCAGCAGGGACAACGCCAAGAACA |
| | TGCTGTACCTGCAGCTGAACAGCCTGAAGACCGAGGACACCGCCATGTACTACTGCGC |
| | CAAGGGCGGCAGCGGCTACGGCGACGCCAGCAGGATGACCAGCCCCGGCAGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGCGGCAGCCTGAGGCTGAGCTGCACCGCCCCCGGCTTCA |
| | CCAGCAACAGCTGCGGCATGGACTGGTACAGGCAGGCCCCCGGCAAGGAGAGGGAGTT |
| | CGTGAGCAGCATCAGCACCGACGGCACCACCGGCTACGCCGACAGCGTGAAGGGCAGG |
| | TTCACCATCAGCAAGGACAAGGCCAAGGACACCGTGTACCTGCAGATGAACAGCCTGA |
| | AGCCCGAGGACACCGGCATGTACAGCTGCAAGACCAAGGACGGCACCATCGCCACCAT |
| | GGAGCTGTGCGACTTCGGCTACTGGGGCCAGGGCACCCAGGTGACCGTGAGCGCTAGC |
| | CACCACCACCACCACCACCACCAC |
| 189 | CAGGTGCAGCTGCAGGAGAGCGGCGGCGGCCTGGTGCAGCCCGGCGGCAGCCTGAGGC |
| | TGAGCTGCGCCGCCAGCGGCTTCACCTTCAGCCTGAGCAGCATGAGCTGGGTGAGGCA |
| | GGCCCCCGGCAAGGGCCTGGAGTGGGTGAGCGCCATCAGCAGCGGCGGCGCCAGCACC |
| | TACTACACCGACAGCGTGAAGGGCAGGTTCACCATCAGCAGGGACAACGCCAAGAACA |
| | TGCTGTACCTGCAGCTGAACAGCCTGAAGACCGAGGACACCGCCATGTACTACTGCGC |
| | CAAGGGCGGCAGCGGCTACGGCGACGCCAGCAGGATGACCAGCCCCGGCAGCCAGGG |
| | CACCCAGGTGACCGTCTCGAGTGGCGGCGGATCCCAGGTGCAGCTGCAGGAGAGCGGC |
| | GGGGCAGCGTGCAGGCCGGCGGCAGCCTGAGGCTGAGCTGCGCCGCCAGCGGCTACC |
| | CCTACAGCAACGGCTACATGGGCTGGTTCAGGCAGGCCCCCGGCAAGGAGAGGGAGGG |
| | CGTGGCCACCATCTACACCGGCGACGGCAGGACCTACTACGCCGACAGCGTGAAGGGC |
| | AGGTTCACCATCAGCAGGGACAACGCCAAGAACACCGTGGACCTGCAGATGAGCAGCC |
| | TGAAGCCCGAGGACACCGCCATGTACTACTGCGCCGCCAGGGCCGCCCCCCTGTACAG |
| | CAGCGGCAGCCCCCTGACCAGGGCCAGGTACAACGTGTGGGGCCAGGGCACCCAGGTG |
| | ACCGTGAGCGCTAGCCACCACCACCACCACCACCACCAC |
| 190 | CAGGTGCAGCTGCAGGAGAGCGGCGGCGGCCTGGTGCAGCCCGGCGGCAGCCTGAGGC |
| | TGAGCTGCGCCGCCAGCGGCTTCACCTTCAGCCTGAGCAGCATGAGCTGGGTGAGGCA |
| | GGCCCCCGGCAAGGGCCTGGAGTGGGTGAGCGCCATCAGCAGCGGCGGCGCCAGCACC |
| | TACTACACCGACAGCGTGAAGGGCAGGTTCACCATCAGCAGGGACAACGCCAAGAACA |
| | TGCTGTACCTGCAGCTGAACAGCCTGAAGACCGAGGACACCGCCATGTACTACTGCGC |
| | CAAGGGCGGCAGCGGCTACGGCGACGCCAGCAGGATGACCAGCCCCGGCAGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGCGGCAGCCTGAGGCTGAGCTGCGTGGCCAGCGCCAGCA |
| | CCTACTGCACCTACGACATGCACTGGTACAGGCAGGCCCCCGGCAAGGGCAGGGAGTT |
| | CGTGAGCGCCATCGACAGCGACGGCACCACCAGGTACGCCGACAGCGTGAAGGGCAG |
| | GTTCACCATCAGCCAGGGCACCGCCAAGAACACCGTGTACCTGCAGATGAACAGCCTG |
| | CAGCCCGAGGACACCGCCATGTACTACTGCAAGACCGTGTGCGTGGTGGGCAGCAGGT |
| | GGAGCGACTACTGGGGCCAGGGCACCCAGGTGACCGTGAGCGCTAGCCACCACCACCA |
| | CCACCACCACCAC |
| 191 | CAGGTGCAGCTGCAGGAGAGCGGCGGGGGCCTGGTGCAGCCCGGCGGCAGCCTGAGGC |
| | TGAGCTGCGCCGCCAGCGGCTTCACCTTCAGCCTGAGCAGCATGAGCTGGGTGAGGCA |
| | GGCCCCCGGCAAGGGCCTGGAGTGGGTGAGCGCCATCAGCAGCGGCGGCGCCAGCACC |
| | TACTACACCGACAGCGTGAAGGGCAGGTTCACCATCAGCAGGGACAACGCCAAGAACA |
| | TGCTGTACCTGCAGCTGAACAGCCTGAAGACCGAGGACACCGCCATGTACTACTGCGC |
| | CAAGGGCGGCAGCGGCTACGGCGACGCCAGCAGGATGACCAGCCCCGGCAGCCAGGG |
| | CACCCAGGTGACCGTCTCGAGTGGCGGCGGATCCCAGGTGCAGCTGCAGGAGAGCGGC |
| | GGCGGCAGCGTGCAGGCCGGCGGCAGCCTGACCCTGAGCTGCGCCGCCAGCGAGTACG |
| | CCTACAGCACCTGCAACATGGGCTGGTACAGGCAGGCCCCCGGCAAGGAGAGGGAGCT |
| | GGTGAGCGCCTTCATCAGCGACGGCAGCACCTACTACGCCGACAGCGTGAAGGGCAGG |
| | TTCACCATCACCAGGGACAACGCCAAGAACACCGTGTACCTGCAGATGAACAGCCTGA |
| | AGCCCGAGGACACCGCCATCTACTACTGCAGCGCCAACTGCTACAGGAGGCTGAGGAA |
| | CTACTGGGGCCAGGGCACCCAGGTGACCGTGAGCGCTAGCCACCACCACCACCACCAC |
| | CACCAC |
| 192 | CAGGTGCAGCTGCAGGAGAGCGGCGGCGGCCTGGTGCAGCCCGGCGGCAGCCTGAGGC |
| | TGAGCTGCGCCGCCAGCGGCTTCACCTTCAGCCTGAGCAGCATGAGCTGGGTGAGGCA |
| | GGCCCCCGGCAAGGGCCTGGAGTGGGTGAGCGCCATCAGCAGCGGCGGCGCCAGCACC |
| | TACTACACCGACAGCGTGAAGGGCAGGTTCACCATCAGCAGGGACAACGCCAAGAACA |
| | TGCTGTACCTGCAGCTGAACAGCCTGAAGACCGAGGACACCGCCATGTACTACTGCGC |
| | CAAGGGCGGCAGCGGCTACGGCGACGCCAGCAGGATGACCAGCCCCGGCAGCCAGGG |
| | CACCCAGGTGACCGTCTCGAGTGGCGGCGGATCCCAGGTGCAGCTGCAGGAGAGCGGC |
| | GGCGGCCTGGTGCAGCCCGGCGGCAGCCTGAGGCTGAGCTGCACCGCCAGGGGCCTGA |
| | CCTTCGACGACAGCGTGATGGGCTGGTTCAGGCAGGCCCCCGGCAAGGGCAGGGAGGC |
| | CGTGAGCTGCATCAGCAGCAGCGGCGCCAACGCCTTCTACGCCGACAGCGTGAAGGGC |
| | AGGTTCACCATCAGCAGGGACAACGCCAAGAACACCCTGTACCTGCAGATGAACAGCC |
| | TGAAGCCCGAGGACACCGCCACCTACTACTGCAAGAGGGGCCACGCCTGCGCCGGCTA |
| | CTACCCCATCCCCTACGACGACTACTGGGGCCAGGGCACCCAGGTGACCGTGAGCGCT |
| | AGCCACCACCACCACCACCACCACCAC |

### Additional IL27R Binding Proteins

Further, additional IL27R binding proteins can include three CDR sequences as underlined in an anti-IL27Rα V_{H}H antibody listed in Table 4 below, and three CDR sequences as underlined in an anti-gp130 V_{H}H antibody of any one of SEQ ID NOS: 232-237. Additional IL27R binding proteins can include an anti-IL27Rα V_{H}H antibody (e.g., any one of the sequences listed in Table 4 below) and an anti-gp130 V_{H}H antibody (e.g., any one of SEQ ID NOS: 232-237. In some embodiments, the binding protein comprises the anti-IL27Rα V_{H}H antibody at the N-terminus and the anti-gp130 V_{H}H antibody at the C-terminus. In some embodiments, the binding protein comprises the anti-gp130 V_{H}H antibody at the N-terminus and the anti-IL27Rα V_{H}H antibody at the C-ferminus. In some embodiments, the binding protein comprises a linker (e.g., any one of SEQ ID NOS: 85-108 (e.g., SEQ ID NO:108)) between the anti-IL27Rα V_{H}H antibody and the anti-gp130 V_{H}H antibody. In certain embodiments, the binding protein comprises a purification tag, such as as a six-histidine peptide (His): (SEQ ID NO: 1531) (His-tag).

TABLE 4

| SEQ | |
|---|---|
| ID | |
| NO | Sequence |
| 258 | QVQLQESGGGSVQVGGSLRLSCAASGFTFSSYPMSWVRQAPGKGLEWISTISAGGDTILYA |
| | DSVKGRFTSSRDNAKNTLYLQLNSLKTEDTAIYYCAKRIDCNSGYCYRRNYWGQGTQVTV |
| | SS |
| 259 | QVQLQESGGGSVQAGGSLRLSCRASGSTYSNYCLGWFRQITGKEREGVAVINWVGGMLYF |
| | ADSVKGRFTVSQDQAKNTLYLQMNSLKPEDTAMYYCAAESVSSFSCGGWLTRPDRVPYW |
| | GQGTQVTVSS |
| 260 | QVQLQESGGGSVQAGGSLRLSCVASGYVSCDYFLPSWYRQAPGKEREFVSIIDGTGSTSYA |
| | ASVKGRFTASQDKGKNIAYLQMNSLKPEDTAMYYCKASCVRGRGISEYWGQGTQVTVSS |
| 261 | QVQLQESGGGLVQPGGSLRLSCAASGFTFSHSGMSWVRQAPGKGLEWVSTINSGGASTYY |
| | TDSVKGRFTISRDNAKNMLYLQLNSLKTEDTAMYYCAKGGSGYGDASRMTSPGSQGTQV |
| | TVSS |
| 262 | QVQLQESGGGLVQPGGSLRLSCAASGFTFSLSGMSWVRQAPGKGLEWVSAISSGGASTYY |
| | TDSVKGRFTISRDNAKNILYLQLNSLKTEDTAMYYCAKGGSGYGDASRMTSPGSQGTQVT |
| | VSS |
| 263 | QVQLQESGGGSVQAGGSLRLSCVASGYVSCDYFLPSWYRQAPGKEREFVSIIDGIGSTSYA |
| | ASVKGRFTASEDKGKNIAYLQMNSLKPEDTAMYYCKASCVRGRAVSEYWGQGTQVTVSS |
| 264 | QVQLQESGGGSVQAGGSLRLSCTASGYVSCDYFLPSWYRQAPGKEREFVSVIDGTGSTSYA |
| | ASVKGRFTASQDKGKNIAYLQMNSLKPEDTAMYYCKASCVRGRAISEYWGQGTQVTVSS |
| 265 | QVQLQESGGGLVQPGGSLRLSCAASGFTFSSYPMSWVRQAPGKGLEWISTISAGGDTTLYA |
| | DSVKGRFTSSRDNAKNTLYLQLNSLKTEDTAIYYCAKRIDCNSGYCYRRNYWGQGTQVTV |
| 266 | QVQLQESGGGSVQAGGSLRLSCRASGSTYSNYCLGWFRQSTGKEREGVAVINWVGGMLY |
| | FADSVKGRFTVSQDHAKNTVTLQMNSLKPEDTAMYYCAAESVSSFSCGGWLTRPGRYPY |
| | WGQGTQVTVSS |
| 267 | QVQLQESGGGSVQAGESLRLSCRASGSTYSNYCLGWFRQITGKEREGVAVINWVGGMLYF |
| | ADSVKGRFTVSQDQAKNTVYLEMNSLKPEDTAMYYCATESVSSFSCGGWLTRPDRVPYW |
| | GQGTQVTVSS |
| 268 | QVQLQESGGGSVQAGGSLRLSCVASGYVSCDYFLPSWYRQAPGKEREFVSIIDGTGSTSYA |
| | ASVKGRFTASQDRGKNIAYLQMNSLKPEDTAMYYCKASCVRGRTISEYWGQGTQVTVSS |
| 269 | QVQLQESGGGSVQAGGSLRLSCVASGYVSCDYFLPSWYRQAPGKEREFVSIIDGTGSTSYA |
| | ASVKGRFTASQDKGKNIAYLQMNSLKPEDTAMYYCKASCVRGRAISEYWGQGTQVTVSS |
| 270 | QVQLQESGGGSVQAGGSLRLSCRASGSTYSNYCLGWFRQITGKEREGVAVINWVGGMLYF |
| | ADSVKGRFTVSQDQAKNTVYLQMNSLKPEDTAMYYCAAESASSFSCGGWLTRPDRVPYW |
| | GQGTQVTVSS |
| 271 | QVQLQESGGGLVQPGGSLRLSCAASGFTFSLSGMSWVRQAPGKGLEWVSAISSGGASTYY |
| | TDSVKGRFTISRDNAKNMLYLQLNSLKTEDTAMYYCAKGGSGYGDASRMTSPGSQGTQV |
| | TVSS |
| 272 | QVQLQESGGGLVQPGGSLRLSCAASGFTFSSYPMSWVRQAPGKGLEWVSTISSGGDTTLYA |
| | DSVKGRFTSSRDNAKNILYLQLNSLKTEDTAMYYCAKRIDCNSGYCYKRSYWGQGTQVT |
| | VSS |
| 273 | QVQLQESGGGSVQAGGSLRLSCRASGSTYSNYCLGWFRQTTGKEREGVAVINWVGGMLY |
| | FADSVKGRFTVSQDQAKNTVYLQMNSLKPEDTAMYYCAAESVSSFSCGGWLTRPDRVPY |
| | WGQGTQVTVSS |
| 274 | QVQLQESGGGSVQAGGSLRLSCRASRSPYGNYCLGWFRQSTGKEREGVAVINWVGGMLY |
| | FADSVKGRFTVSQDHAKNTVTLQMNSLKPEDTAMYYCAAESVSSFSCGGWLTRPDRVPY |
| | WGQGTQVTVSS |

An IL27R binding protein can comprise three CDR sequences as underlined in an anti-IL27Rα V_{H}H antibody and three CDR sequences as underlined in an anti-gp130 V_{H}H antibody as described in each row of Table 5 below. In some embodiments, an IL27R binding protein comprises an anti-IL27Rα V_{H}H antibody and an anti-gp130 V_{H}H antibody as described in each row of Table 5 below. In some embodiments, the binding protein comprises the anti-IL27Rα V_{H}H antibody at the N-terminus and the anti-gp130 V_{H}H antibody at the C-terminus. In some embodiments, the binding protein comprises the anti-gp130 V_{H}H antibody at the N-terminus and the anti-IL27Rα V_{H}H antibody at the C-terminus. In some embodiments, the binding protein comprises a linker (e.g., any one of SEQ ID NOS: 85-108 (e.g., SEQ ID NO: 108)) between the anti-IL27Rα V_{H}H antibody and the anti-gp130 V_{H}H antibody. In certain embodiments, the binding protein comprises a purification tag, such as as a six-histidine peptide (His), (SEQ ID NO: 1531) (His-tag).

TABLE 5

| SEQ ID NO of | SEQ ID NO of |
|---|---|
| Anti-IL27Rα V_{H}H antibody | Anti-gp130 V_{H}H antibody |
| 258 | 232 |
| 259 | 232 |
| 260 | 232 |
| 261 | 232 |
| 262 | 232 |
| 263 | 232 |
| 264 | 232 |
| 265 | 232 |
| 266 | 232 |
| 267 | 232 |
| 268 | 232 |
| 269 | 232 |
| 270 | 232 |
| 271 | 232 |
| 272 | 232 |
| 273 | 232 |
| 274 | 232 |
| 258 | 233 |
| 259 | 233 |
| 260 | 233 |
| 261 | 233 |
| 262 | 233 |
| 263 | 233 |
| 264 | 233 |
| 265 | 233 |
| 266 | 233 |
| 267 | 233 |
| 268 | 233 |
| 269 | 233 |
| 270 | 233 |
| 271 | 233 |
| 272 | 233 |
| 273 | 233 |
| 274 | 233 |
| 258 | 234 |
| 259 | 234 |
| 260 | 234 |
| 261 | 234 |
| 262 | 234 |
| 263 | 234 |
| 264 | 234 |
| 265 | 234 |
| 266 | 234 |
| 267 | 234 |
| 268 | 234 |
| 269 | 234 |
| 270 | 234 |
| 271 | 234 |
| 272 | 234 |
| 273 | 234 |
| 274 | 234 |
| 258 | 235 |
| 259 | 235 |
| 260 | 235 |
| 261 | 235 |
| 262 | 235 |
| 263 | 235 |
| 264 | 235 |
| 265 | 235 |
| 266 | 235 |
| 267 | 235 |
| 268 | 235 |
| 269 | 235 |
| 270 | 235 |
| 271 | 235 |
| 272 | 235 |
| 273 | 235 |
| 274 | 235 |
| 258 | 236 |
| 259 | 236 |
| 260 | 236 |
| 261 | 236 |
| 262 | 236 |
| 263 | 236 |
| 264 | 236 |
| 265 | 236 |
| 266 | 236 |
| 267 | 236 |
| 268 | 236 |
| 269 | 236 |
| 270 | 236 |
| 271 | 236 |
| 272 | 236 |
| 273 | 236 |
| 274 | 236 |
| 258 | 237 |
| 259 | 237 |
| 260 | 237 |
| 261 | 237 |
| 262 | 237 |
| 263 | 237 |
| 264 | 237 |
| 265 | 237 |
| 266 | 237 |
| 267 | 237 |
| 268 | 237 |
| 269 | 237 |
| 270 | 237 |
| 271 | 237 |
| 272 | 237 |
| 273 | 237 |
| 274 | 237 |

Further, an IL27R binding protein can comprise a mouse Anti IL27Rα V_{H}H antibody and a mouse Anti gp130 V_{H}H antibody. In some instances, due to sequence or structural similarities between the extracellular domains of receptors from various mammalian species, immunization with an antigen derived from a IL27Rα or gp130 of a first mammalian species may provide antibodies which specifically bind to receptors of one or more additional mammalian species. Such antibodies are termed “cross reactive.” For example, immunization of a camelid with a human derived antigen (e.g., the hIL27Rα-ECD) may generate antibodies that are cross-reactive the murine and human receptors. Evaluation of cross-reactivity of antibody with respect to the receptors derived from other mammalian species may be readily determined by the skilled artisan, for example using the methods relating to evaluation of binding affinity and/or specific binding described elsewhere herein such as flow cytometry or SPR. Consequently, the use of the term “human IL27Rα VHH” or “hIL27Rα VHH” merely denotes that the species of the IL27Rα antigen used for immunization of the camelid from which the VHH was derived was the human IL27Rα but should not be understood as limiting with respect to the specific binding affinity of the VHH for IL27Rα molecules of other mammalian species. Similarly, the use of the term “mouse IL27Rα VHH” or “mIL27RαVHH” merely denotes that the species of the IL27Rα antigen used for immunization of the camelid from which the VHH was derived was the murine IL27Rα but should not be understood as limiting with respect to the specific binding affinity of the VHH for IL27Rα molecules of other mammalian species. In some embodiments, an IL27R binding protein can comprise three CDR sequences as underlined in a mouse anti-IL27Rα V_{H}H antibody sequence listed below and three CDR sequences as underlined in a mouse anti-gp130 V_{H}H antibody sequence listed below. In some embodiments, an IL27R binding protein comprises a mouse anti-IL27Rα V_{H}H antibody listed below and a mouse anti-gp130 V_{H}H antibody listed below. In some embodiments, the binding protein comprises the mouse anti-IL27Rα V_{H}H antibody at the N-terminus and the mouse anti-gp130 V_{H}H antibody at the C-terminus. In some embodiments, the binding protein comprises the mouse anti-gp130 V_{H}H antibody at the N-terminus and the mouse anti-IL27Rα V_{H}H antibody at the C-terminus. In some embodiments, the binding protein comprises a linker (e.g., any one of SEQ ID NOS: 85-108 (e.g., SEQ ID NO:108)) between the mouse anti-IL27Rα V_{H}H antibody and the mouse anti-gp130 V_{H}H antibody. In certain embodiments, the binding protein comprises a purification tag, such as as a six-histidine peptide (His) (SEQ ID NO: 1531) (His-tag).

### Examples of Mouse Anti-IL27Rα V_{H}H Antibody Sequences:

>mouse anti-IL27Rα V_{H}H antibody_
SEQ ID NO: 275
QVQLQESGGGSVQAGGSLRLSCAASKNSNFMGWFRQAPGKEREGVAAMMTKNNN
TYYADSVKGRFTISHDNAKNTVYLQMDSLKPEDTAVYYCAAVYRTRRLRVLEAAN
FDYWGQGTQVTVSS
>mouse anti-IL27Rα V_{H}H antibody_
SEQ ID NO: 276
QVQLQESGGGSVQAGGSLRLSCTASGYTSSRYCMGWFRQTPGKKREGVAAIYTGG
GTTFYHGSVKGRFTISQDNTTNTVYLQMHNLKPEDTAMYYCAAGPVTRACDEYNY
WGQGTQVTVSS
>mouse anti-IL27Rα V_{H}H antibody_
SEQ ID NO: 277
QVQLQESGGGSVQAGGSLRLSCAGSGYSLSNYCMGWFRQAPGQGREGVASLRFVS
GATFYADSVKGRFTIAQDNAKNTLYLQMNSLKPEDTAMYYCGIKSRGICGGRLVDV
DFGNWGQGTQVTVSS
>mouse anti-IL27Rα V_{H}H antibody_
SEQ ID NO: 278
QVQLQESGGGSVQAGGSLRLSCAASGYSINRMGWFRQAPGKEREGVAAISIGGGQT
YYADSVKGRFTISQDNAKNTVDLQMNSLKPEDTAMYYCAAGLVYGEAWLDSRHY
NKWGQGTQVTVSS
>mouse anti-IL27Rα V_{H}H antibody_
SEQ ID NO: 279
QVQLQESGGGSVQAGGSLRLSCAVSGDSTYSMGWFRQPPGKEREGVAAIAKDGITI
HADSVKGRFTISKDNAKNTLYLQMNSLKPEDTAMYYCAAHRPYGPPLNPRWYTYW
GQGTQVTVSS
>mouse anti-IL27Rα V_{H}H antibody_
SEQ ID NO: 280
QVQLQESGGGSVQAGGSLRLSCAASGYTYSSYCMAWFRQAPGKEREGVAAIDSDGS
TSYADSVKGRFTISKDNAKNTLYLQMNSLKPEDTAMYYCAAASGRCLGPGIRSLIW
GQGTQVTVSS
>mouse anti-IL27Rα V_{H}H antibody_
SEQ ID NO: 281
QVQLQESGGGSVQAGGSLRLSCAVSGDSTYSMGWFRQPPGKEREGVAAITKDITIHA
DSVKGRFTISKDNAKNTLYLQMNSLKPEDTAMYYCAAHRPYGPPLNPRWYTYWGQ
GTQVTVSS
>mouse anti-IL27Rα V_{H}H antibody_
SEQ ID NO: 282
QVQLQESGGGSVQAGGSLRLSCAVSGDSTYSMGWFRQPPGKEREGVAAIPTDGITIH
ADSVKGRFTISKDNAKNTLYLQMNSLKPEDTAMYYCAAHRPYGPPLNPRWYTYWG
QGTQVTVSS
>mouse anti-IL27Rα V_{H}H antibody_
SEQ ID NO: 283
QVQLQESGGGSVQAGGSLRLSCAVSGDSTYSMGWFRQPPGKEREGVAAIAKDGITI
HADSVKGRFTISKDNAKNTLYLQMSSLKPEDTAMYYCAAHRPYGPPLNPRWYTYW
GQGTQVTVSS
>mouse anti-IL27Rα V_{H}H antibody_
SEQ ID NO: 284
QVQLQESGGGSVQAGGSLRLSCAVSGDSTYSMGWFRQPPGKEREGVAAIGKDGITI
HADSVKGRFTISKDNAKNTLYLQMNSLKPEDTAMYYCAAHRPYGPPLNPRWYTYW
GQGTQVTVSS
>mouse anti-IL27Rα V_{H}H antibody_
SEQ ID NO: 285
QVQLQESGGGSVQAGGSLRLSCAVSGDSTYSMGWFRQPPGKEREGVAAITKDITIHA
DSVKGRFTISKDNAKNTLYLQMNSLRPEDTAMYYCAAHRPYGPPLNPRWYTYWGQ
GTQVTVSS
>mouse anti-IL27Rα V_{H}H antibody_
SEQ ID NO: 286
QVQLQESGGGSVQTGGSLRLSCAASGYSINRMAWFRQAPGKEREGVAAISIGGDRT
YYADSVKGRFTISQDNAKHTVDLQMNSLKPEDTAMYYCAAGLVYGEAWLDSRHY
NKWGQGTQVTVSS
>mouse anti-IL27Rα V_{H}H antibody_
SEQ ID NO: 287
QVQLQESGGGSVQAGGSLRLSCAASGYSINRMGWFRQAPGKEREGVAAISIGGGRT
YYADSVKGRFTISQDNAKNTVDLQMNSLKPEDTAMYYCAAGLVYGEAWLDSRHY
NKWGQGTQVTVSS
>mouse anti-IL27Rα V_{H}H antibody_
SEQ ID NO: 288
QVQLQESGGGSVQAGGSLRLSCAVSGDSTYSMGWFRQPPGKEREGVAAITKDGITIH
ADSVKGRFTISGDNAKNTLYLQMNNLKPEDTAMYYCAAHRPYGPPLNPRWYTYWG
QGTQVTVSS

### Examples of Mouse Anti-Gp130 V_{H}H Antibody Sequences:

>mouse anti-gp130 V_{H}H antibody_
SEQ ID NO: 289
QVQLQESGGGSVQAGGSLRLSCVISGFTYRQTFMGWFRQVLGKEREGVAAISTGGG
STVYADSVKGRFTISQDSSKNTVYLEMNGLKLEDTGMYYCAASTVITSVSINRGLYQ
YWGQGTQVTVSS
>mouse anti-gp130 V_{H}H antibody_
SEQ ID NO: 290
QVQLQESGGGSVQAGGSLRLSCAISGFTYRQTFMGWFRQVVGKEREGVAAISTGGG
STVYADSVKGRFTISQDSSKDTVYLEMNGLKLEDTGMYYCAASTVITSESINRNLYQ
YWGQGTQVTVSS
>mouse anti-gp130 V_{H}H antibody_
SEQ ID NO: 291
QVQLQESGGGLVQPGGSLRLSCAASGFTLSTYWIYWVRQAPGKGPEWVSTVSRSGG
TTYYADSVNGRFTISRDNAKNTVYLQMNNLKPEDAAVYYCLASVSNLGWPPVRAPS
PTGQGTQVTVSS
>mouse anti-gp130 V_{H}H antibody_
SEQ ID NO: 292
QVQLQESGGGLVQPGGSLRLSCAASGFTFSSYAMSWVRQAPGKGLEWVSAINSGGA
GTYYTDSVKGRFTISRDNAKNTLYLQLNSLKTEDTAMYYCAKHVTGDYDPSLRHGY
NVWSQGTQVTVSS
>mouse anti-gp130 V_{H}H antibody_
SEQ ID NO: 293
QVQLQESGGGSVQAGGSLRLSCVISGFTYRQTFMGWFRQVVGKEREGVAAISTGGG
STIYADSVKGRFTISQDSSKDTVYLEMNGLKLEDTGMYYCAASTVITSESINRNLYQY
WGQGTQVTVSS
>mouse anti-gp130 V_{H}H antibody_
SEQ ID NO: 294
QVQLQESGGGLVQPGGSLRLSCAASGFTLSTYWMYWVRQAPGKGPEWVSAVSRGG
FNTYYADSVKGRFTISRDNAKNTVYLQMNSLKPEDTAVYYCMSSVSFYGWPPDRVP
SPTGQGTQVTVSS
>mouse anti-gp130 V_{H}H antibody_
SEQ ID NO: 295
QVQLQESGGGLVQPGESLRLSCAASGFTFSSYAMSWVRQAPGKGLEWVSAINSGGA
GTYYTDSVKGRFTISRDNAKNTLYLQLNSLKTEDTAMYYCAKHVTGDYDPSLRYGY
NVWSQGTQVTVSS
>mouse anti-gp130 V_{H}H antibody_
SEQ ID NO: 296
QVQLQESGGGSVQAGGSLRLSCVISGFTYRPTFMGWFRQVLGKEREGVAAITTGGG
STVYADSVKGRFTISQDSSKNTVYLEMNGLKLEDTGMYYCAATTVITSVSINRNLYQ
YWGQGTQVTVSS
>mouse anti-gp130 V_{H}H antibody_
SEQ ID NO: 297
QVQLQESGGGSVQAGGSLRLSCGISGFTYRPTFMGWFRQVLGKEREGVAAISTGGGS
SVYADSVKGRFTVSQDSSKNTVYLEMNGLKLEDIGMYYCAASTVITSVSINRGLYQ
YWGQGTQVTVSS
>mouse anti-gp130 V_{H}H antibody_
SEQ ID NO: 298
QVQLQESGGGLVQPGGSLRLSCTASGFTFRNYAMSWVRQAPGKGLEWVSAINSGG
GSTYYADSVKGRFTISRDNAKNTLYLRLHSLKTEDTAMYYCAKHVTGDYDPSLRYE
YNYWSQGTQVTVSS
>mouse anti-gp130 V_{H}H antibody_
SEQ ID NO: 299
QVQLQESGGGLVQPGGSLRLSCAASGFTFRNYAMSWVRQAPGKGLEWVSAINSGG
GSTYYADSVKGRFTISRDNAKNTLYLQLNSLKTEDTAMYYCTKHVTGDYDPSLRYE
YNYWSQGTQVTVSS
>mouse anti-gp130 V_{H}H antibody_
SEQ ID NO: 300
QVQLQESGGGLVQPGGSLRLSCAASGFTFSSYAMSWVRQAPGKGLEWVSAINSGGG
STYYADSVKGRFTISRDNAKNTLYLQLNSLKTEDTAMYCCAKHVTGDYDPSLRYGY
NCWGPGTQVTVSS
>mouse anti-gp130 V_{H}H antibody_
SEQ ID NO: 301
QVQLQESGGGSVQPGGSLRLSCAASGFTFSTYDMSWVRQAPGKGLEWVSTINYSGS
STYYVDSVLGRFTIARDNAKNTLYLQMNNLQTEDTAVYYCASVKERRSNGHPIVFG
DRGQGTQVTVSS
>mouse anti-gp130 V_{H}H antibody_
SEQ ID NO: 302
QVQLQESGGGSVQAGGSLRLSCVISGFTYKQTFMGWFRQVPGKEREGVAAISTGGG
STVYADSVKGRFTISQDSSKNTVYLEMNGLKLEDTGMYYCAASTVITSVSINRGLYQ
YWGQGTQVTVSS
>mouse anti-gp130 V_{H}H antibody_
SEQ ID NO: 303
QVQLQESGGGSVQAGGSLRLSCVISGFTYRQTFMGWFRQVVGKEREGVAAISTGGG
STVYADSVKGRFTISQDSSKDTVYLEMNGLKLEDTGMYYCAASTVITSESIYRNLYQ
YWGQGTQVTVSS
>mouse anti-gp130 V_{H}H antibody_
SEQ ID NO: 304
QVQLQESGGGLVQPGGSLRLSCAASGFTFRNYAMSWVRQAPGKGLEWVSAINSGG
GSTYYADSVKGRFTISRDNAKNTLYLQMNSLKPEDTAMYYCAKHVTGDYDPSLRY
EYNYWSQGTQVTVSS
>mouse anti-gp130 V_{H}H antibody_
SEQ ID NO: 305
QVQLQESGGGLVQPGGSLRLSCAASGFTFRNYAMSWVRQAPGKGLEWVSAINSGG
GSTYYADSVKGRFTISRDNAKNTLYLQLNSLKTEDTAMYYCAKHVTGDYDPSLRYE
YAYWSQGTQVTVSS
>mouse anti-gp130 V_{H}H antibody_
SEQ ID NO: 306
QVQLQESGGGSVQAGGSLRLSCAASGFTFSSYAMSWVRQAPGKGLEWVSAINSGGG
STYYADSVKARFTISRDNAKNTLYLQLNSLKTEDTAMYYCAKHVTGDYDPSLRYDY
NYWGQGTQVTVSS
>mouse anti-gp130 V_{H}H antibody_
SEQ ID NO: 307
QVQLQESGGGSVQAGGSLRLSCGISGFTYRPTFMGWFRQVLGKEREGVAAISTGGGS
SVYADSVKGRFTVSQDSSKNTVYLEMNGLKLEDTGMYYCAASTVITSVSINRALYQ
YWGQGTQVTVSS
>mouse anti-gp130 V_{H}H antibody_
SEQ ID NO: 308
QVQLQESGGGLVQPGGSLRLSCAASGFTFSSYAVSWVRQAPGKGLEWVSTINSGGG
STYYADSVKGRFTISRDNAKNTLYLQLNSLKTEDTAMYYCTKHVTGDYDPSLRYEY
NVWSQGTQVTVSS
>mouse anti-gp130 V_{H}H antibody_
SEQ ID NO: 309
QVQLQESGGGSVQAGGSLRVSCQISGFTYRQTFMGWFRQVPGKEREGVAAISTGGG
STVYADSVKGRFTISQDSSKNTVYLEMNGLKLEDTGMYYCAASTVITSPSINRNLYQ
YWGQGTQVTVSS
>mouse anti-gp130 V_{H}H antibody_
SEQ ID NO: 310
QVQLQESGGGLVQPGGSLRLSCAVSGFTFSNYAMKWVRQAPGKGLEWVSSISGGG
GATYYADSVKGRFTISRDNTKNTLYLQMNSLKTEDTAVYYCAAQNLDYRGQGTQV
TVSS
>mouse anti-gp130 V_{H}H antibody_
SEQ ID NO: 311
QVQLQESGGGLVQPGGSLRLSCTASGFTFNSAHMKWERQPPGKGLEWVSFITPGGA
STGYADSVKGRFTISRDNAKNTLYLQMNNLKTEDTAVYYCATGGLRGQGTQVTVSS
>mouse anti-gp130 V_{H}H antibody_
SEQ ID NO: 312
QVQLQESGGGSVQAGGSLRLSCVISGFTYRPTFMGWFRQVLGKEREGVAAITTGGG
STLYADSVKGRFTISQDSSKNTVYLEMNGLKLEDTGMYYCAATTVITSVSINRNLYQ
YWGQGTQVTVSS
>mouse anti-gp130 V_{H}H antibody_
SEQ ID NO: 313
QVQLQESGGGSVQAGGSLRLSCVISGFTYKQTFMGWFRQVPGKEREGVAAISTGGG
STVYADSVKGRFTISQDSSKNTVYLEMNGLKLEDTGMYYCAASTVITSVTINRGLYQ
YWGQGTQVTVSS
>mouse anti-gp130 V_{H}H antibody_
SEQ ID NO: 314
QVQLQESGGGSVQAGGSLRLSCVISGFVYKQTFMGWFRQVPGKEREGVAAISTGGG
STVYADSVKGRFTISQDSSKNTVYLEMNGLKLEDTGMYYCAASTVITSVSINRGLYQ
YWGQGTQVTVSS
>mouse anti-gp130 V_{H}H antibody_
SEQ ID NO: 315
QVQLQESGGGSVQAGGSLRVSCVISGFTYRQTFMGWFRQVPGKEREGVAAISTGGG
STVYADSVKGRFTISQDSSKNTVYLEMNGLKLEDTGMYYCAASTVITSVSINRGLYQ
YWGQGTQVTVSS
>mouse anti-gp130 V_{H}H antibody_
SEQ ID NO: 316
QVQLQESGGGLVQPGGSLRLSCAASGFTFSSYAMSWVRQAPGKGLEWVSAINSGGG
STYYADSVKGRFTISRDNAKSTLYLQLNSLKTEDTAMYYCTKHVTGDYDPSLRYEY
NYWSQGTQVTVSS
>mouse anti-gp130 V_{H}H antibody_
SEQ ID NO: 317
QVQLQESGGGLVQPGGSLRLSCAASGFTFSSYAMSWVRQAPGKGLEWVSAINSGGA
STYYADSVKGRFTISRDNAKNILYLQLNSLKTEDTAMYCCAKHVTGDYDPSLRYEY
NCWGPGTQVTVSS
>mouse anti-gp130 V_{H}H antibody_
SEQ ID NO: 318
QVQLQESGGGLVQPGGSLRLSCAASGFTFSSYAMSWVRQAPGKGLEWVSAINSGGG
STYYADSVKGRFTISRDNAKNTLYLQLNSLKTEDTAMYYCAKHVITGDYDPSLRYEY
NYWSQGTQVTVSS
>mouse anti-gp130 V_{H}H antibody_
SEQ ID NO: 319
QVQLQESGGGSVQAGGSLRLSCVISGFTYRQTFMGWFRQVVGKEREGVAAISTGGG
STVYADSVKGRFTISQDSSKDTVYLEMNGLKLEDTGMYYCAASTVITSESINRNLYQ
YWGQGTQVTVSS
>mouse anti-gp130 V_{H}H antibody_
SEQ ID NO: 320
QVQLQESGGGSVQAGGSLRVSCVVSGFTYRQTFMGWFRQVPGKEREGVAAISTGG
GSTVYADSVKGRFTISQDSSKNTVYLEMNGLKLEDIGMYYCAASTVITSVSINRNLY
QYWGQGTQVTVSS
>mouse anti-gp130 V_{H}H antibody_
SEQ ID NO: 321
QVQLQESGGGLVQPGGSLRLSCAASGFTFSSYAMSWVRQAPGKGLEWVSAINSGGG
STYYADSVKGRFTISRDNAKNTMYLQLNSLKTEDTAMYYCAKHVAGDYDPSLRYE
WHVWGQGTQVTVSS
>mouse anti-gp130 V_{H}H antibody_
SEQ ID NO: 322
QVQLQESGGGLVQPGGSLRLSCAASGFTFRSYAMSWVRQAPGKGLEWVSAINSGGG
STYYADSVKARFTISRDNAKNTLYLQLNSLKTEDTAMYYCAKHVTGDYDPSLRYEY
NYWGQGTQVTVSS
>mouse anti-gp130 V_{H}H antibody_
SEQ ID NO: 323
QVQLQESGGGLVQPGGSLRLSCAASGFTFSSYAMSWVRQAPGKGLEWVSAINSGGG
STYYADSVKGRFTISRDNAKNTLYLQLNSLKTEDTAMYYCAKHITGDYDPSLRYEY
NYWGQGTQVTVSS
>mouse anti-gp130 V_{H}H antibody_
SEQ ID NO: 324
QVQLQESGGGSVQAGGSLRLSCVISGFTYRQIFMGWFRQVVGKEREGVAAISTGGG
SMVYADSVKGRFTISQDSSKDTVYLEMNGLKLEDTGMYYCAASTVITSESINRNLYQ
YWGQGTQVTVSS
>mouse anti-gp130 V_{H}H antibody_
SEQ ID NO: 325
QVQLQESGGGPVQAGGSLRLSCVISGFTYRQTFMGWFRQVPGKEREGVAAISTGGG
STVYADSVKGRFTISQDSSKNTVYLEMNGLKLEDTGMYYCAASTVITSESINRGLYQ
YWGQGTQVTVSS
>mouse anti-gp130 V_{H}H antibody_
SEQ ID NO: 326
QVQLQESGGGSVQAGGFLRLSCAFSGYTGCMGWFRQGPGQEREGVASINDGGSLTY
ADSVKGRFTISKDNAKKTLDLQMNTLKPEDTAMYYCAASLSYCLNPTLRVDGYNY
WGQGTQVTVSS
>mouse anti-gp130 V_{H}H antibody_
SEQ ID NO: 327
QVQLQESGGGSVQAGGSLRLSCVISGLTYKQTFMGWFRQVPGKEREGVAAISTGGG
STVYADSVKGRFTISQDNSKNTVYLEMNGLKLEDTGMYYCAASTVITSVSINRYLYQ
WWGQGTQVTVSS
>mouse anti-gp130 V_{H}H antibody_
SEQ ID NO: 328
QVQLQESGGGLVQPGGSLRLSCAASGFTFSNYAMSWVRQAPGKGVEWVSAINSGGS
VFYADSVKGRFTISRDNAKNTLYLQLSSLKTEDTAMYYCAKHVTGDYDPSLRYGYN
VWSQGTQVTVSS
>mouse anti-gp130 V_{H}H antibody_
SEQ ID NO: 329
QVQLQESGGGSVQAGGSLRLSCGISGFTYRPTFMGWFRQVLGKEREGVAAISTGGGS
TVYADSVKGRFTISQDSSKDTVYLEMNGLKLEDIGMYYCAASTVITSESINRNLYQY
WGQGTQVTVSS
>mouse anti-gp130 V_{H}H antibody_
SEQ ID NO: 330
QVQLQESGGGLVQPGGSLRLSCAASGFTFSSYAMSWVRQAPGKGLEWVSAINSGGG
STYYADSVKGRFTISRDNAKNTLYLQLNSLKTEDTAMYYCAKHVTGDYDPSLRYGY
NVWSQGTQVTVSS
>mouse anti-gp130 V_{H}H antibody_
SEQ ID NO: 331
QVQLQESGGGLVQPGGSLRLSCAASGFTFSSYAMSWVRQAPGKGLEWVSAINLGGD
TTYYTDSVKGRFTISRDNAKNTLYLQLNSLKTEDTAMYYCAKHVTGDYDPSLRYEY
GYWSQGTQVTVSS
>mouse anti-gp130 V_{H}H antibody_
SEQ ID NO: 332
QVQLQESGGGLVQPGGSLRLSCTASGFTFNSAHLKWERQPPGKGLEWVSFITNGGAS
TGYADSVKGRFTISRDDAKNTLYLQMNNLKTEDTAVYYCATGGLRGQGTQVTVSS
>mouse anti-gp130 V_{H}H antibody_
SEQ ID NO: 333
QVQLQESGGGSVQAGGSLRVSCVISGFTYRQTFMGWFRQVPGKEREGVAAISTGGG
STIYANSVKGRFTISQDSSKNTVYLEMNGLKLEDIGMYYCAASTVITSVSINRGLYQ
YWGQGTQVTVSS
>mouse anti-gp130 V_{H}H antibody_
SEQ ID NO: 334
QVQLQESGGGLVQPGGSLRLSCAASGFTFSSYAMSWVRQAPGKGLEWVSAINSGGG
STYYADSVKGRFTISRDNAKNTLYLQLNSLKTEDTAMYCCAKHITGDYDPSLRYEY
NCWGQGTQVTVSS
>mouse anti-gp130 VuHI antibody_
SEQ ID NO: 335
QVQLQESGGGSVQAGGSLRLSCVISGFTYKQTFMGWFRQVPGKEREGVAAISTGGG
NTVYADSVKGRFTISQDSSKNTVYLEMNGLKLEDTGMYYCAASTVITSVTVNRGLY
QYWGQGTQVTVSS
>mouse anti-gp130 VHHI antibody_
SEQ ID NO: 336
QVQLQESGGALVQPGGSLRLSCAASGFTFSYYAMKWVRQAPGKGLEWVSSISGGG
GATYYADSVKGRFTISRDNINDTLYLQMNSLKTEDTAVYYCAAQNLDYRGQGTQV
TVSS
>mouse anti-gp130 VHH antibody _
SEQ ID NO: 337
QVQLQESGGGSVQAGGSLRLSCVISGFTYKQTFMGWFRQVPGKEREGVAAISTGGG
STVYADSVKGRFTISQESSKNTVYLEMNGLKLEDTGMYYCAASTVITSVTINRGLYQ
YWGQGTQVTVSS

In some embodiments, a V_{H}H described herein can be humanized to contain human framework regions. Examples of human germlines that could be used to create humanized V_{H}Hs include, but are not limited to, VH3-23 (e.g., UniProt ID: P01764), VH3-74 (e.g., UniProt ID: A0A0B4J1X5), VH3-66 (e.g., UniProt ID: A0A0C4DH42), VH3-30 (e.g., UniProt ID: P01768), VH3-11 (e.g., UniProt ID: P01762), and VH3-9 (e.g., UniProt ID: P01782).

In some embodiments, the IL27R binding protein has a reduced Eₘₐₓ compared to the Eₘₐₓ caused by IL27. Eₘₐₓ reflects the maximum response level in a cell type that can be obtained by a ligand (e.g., a binding protein described herein or the native cytokine (e.g., IL27)). In some embodiments, the IL27R binding protein described herein has at least 1% (e.g., between 1% and 100%, between 10% and 100%, between 20% and 100%, between 30% and 100%, between 40% and 100%, between 50% and 100%, between 60% and 100%, between 70% and 100%, between 80% and 100%, between 90% and 100%, between 1% and 90%, between 1% and 80%, between 1% and 70%, between 1% and 60%, between 1% and 50%, between 1% and 40%, between 1% and 30%, between 1% and 20%, or between 1% and 10%) of the Eₘₐₓ caused by IL27. In other embodiments, the Eₘₐₓ of the IL27R binding protein described herein is greater (e.g., at least 1%, 5%, 10%, 15%, 20%, 25%, 30%, 35%, 40%, 45%, or 50% greater) than the E . . . of the natural ligand, IL27. In some embodiments, by varying the linker length of the IL27R binding protein, the Eₘₐₓ of the IL27R binding protein can be changed. The IL27R binding protein can cause Eₘₐₓ in the most desired cell types, and a reduced Eₘₐₓ in other cell types.

### IV. Linkers

As previously described, the binding domains of the binding proteins of the present disclosure may be joined contiguously (e.g., the C-terminal amino acid of the first V_{H}H in the binding protein to the N-terminal amino acid of the second V_{H}H in the binding protein) or the binding domains of the binding protein may optionally be joined via a linker. A linker is a linkage between two elements, e.g., protein domains. In a bispecific V_{H}H² binding protein described herein, a linker is a linkage between the two V_{H}Hs in the binding protein. A linker can be a covalent bond or a peptide linker. In some embodiments, the two V_{H}Hs in a binding protein are joined directly (i.e., via a covalent bond). The length of the linker between two V_{H}Hs in a binding protein can be used to modulate the proximity of the two V_{H}Hs of the binding protein. By varying the length of the linker, the overall size and length of the binding protein can be tailored to bind to specific cell receptors or domains or subunits thereof. For example, if the binding protein is designed to bind to two receptors or domains or subunits thereof that are located close to each other on the same cell, then a short linker can be used. In another example, if the binding protein is designed to bind to two receptors or domains or subunits there of that are located on two different cells, then a long linker can be used.

In some embodiments, the linker is a peptide linker. A peptide linker can include between 1 and 50 amino acids (e.g., between 2 and 50, between 5 and 50, between 10 and 50, between 15 and 50, between 20 and 50, between 25 and 50, between 30 and 50, between 35 and 50, between 40 and 50, between 45 and 50, between 2 and 45, between 2 and 40, between 2 and 35, between 2 and 30, between 2 and 25, between 2 and 20, between 2 and 15, between 2 and 10, between 2 and 5 amino acids). A linker can also be a chemical linker, such as a synthetic polymer, e.g., a polyethylene glycol (PEG) polymer.

In some embodiments, a linker joins the C-terminus of the first V_{H}H in the binding protein to the N-terminus of the second V_{H}H in the binding protein. In other embodiments, a linker joins the C-terminus of the second V_{H}H in the binding protein to the N-terminus of the first V_{H}H in the binding protein.

Suitable peptide linkers are known in the art, and include, for example, peptide linkers containing flexible amino acid residues such as glycine and serine. In certain embodiments, a peptide linker can contain motifs, e.g., multiple or repeating motifs, of GS, GGS, GGGGS (SEQ ID NO:85), GGGGGS (SEQ ID NO:86), GGSG (SEQ ID NO:87), or SGGG (SEQ ID NO: 88). In certain embodiments, a peptide linker can contain 2 to 12 amino acids including motifs of GS, e.g., GS, GSGS (SEQ ID NO:89), GSGSGS (SEQ ID NO:90), GSGSGSGS (SEQ ID NO:91), GSGSGSGSGS (SEQ ID NO:92), or GSGSGSGSGSGS (SEQ ID NO:93).

In certain other embodiments, a peptide linker can contain 3 to 12 amino acids including motifs of GGS, e.g., GGS, GGSGGS (SEQ ID NO:94), GGSGGSGGS (SEQ ID NO:95), and GGSGGSGGSGGS (SEQ ID NO:96). In yet other embodiments, a peptide linker can contain 4 to 20 amino acids including motifs of GGSG (SEQ ID NO:87), e.g., GGSGGGSG (SEQ ID NO: 97), GGSGGGSGGGSG (SEQ ID NO:98), GGSGGGSGGGSGGGSG (SEQ ID NO:99), or GGSGGGSGGGSGGGSGGGSG (SEQ ID NO:100). In other embodiments, a peptide linker can contain motifs of GGGGS (SEQ ID NO:85), e.g., GGGGSGGGGS (SEQ ID NO: 101) or GGGGSGGGGSGGGGS (SEQ ID NO:102).

Examples of flexible linkers include glycine polymers (G)n, glycine-alanine polymers, alanine-serine polymers, glycine-serine polymers (for example, (GmSo)n (SEQ ID NO: 1535), (GSGGS)n (SEQ ID NO: 1536), (GmSoGm)n (SEQ ID NO: 1537), (GmSoGmSoGm)n (SEQ ID NO: 1538), (GSGGSm)n (SEQ ID NO: 1539), (GSGSmG)n (SEQ ID NO: 1540), (GGS)nG (SEQ ID NO: 1541) and (GGGSm)n (SEQ ID NO: 1542), and combinations thereof, where m, n, and o are each independently selected from an integer of at least 1 to 20, e.g., 1-18, 216, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Examples of flexible linkers include, but are not limited to GGSG (SEQ ID NO:87), GGSGG (SEQ ID NO:103), GSGSG (SEQ ID NO:104), GSGGG (SEQ ID NO:105), GGGSG (SEQ ID NO:106), and GSSSG (SEQ ID NO:107).

Additional examples of flexible linkers include glycine polymers (G) n or glycine-serine polymers (e.g., (GS) n (SEQ ID NO: 1543), (GSGGS) n (SEQ ID NO: 1544), (GGGS) n (SEQ ID NO: 1545) and (GGGGS) n (SEQ ID NO: 1546), where n=1 to 50, for example, 1, 2, 3, 4, 5, 6, 7, 8, 9, 10, 10-20, 20-30, 30-50). Exemplary flexible linkers include, but are not limited to GGGS (SEQ ID NO:108), GGGGS (SEQ ID NO:85), GGSG (SEQ ID NO:87), GGSGG (SEQ ID NO:103), GSGSG (SEQ ID NO:104), GSGGG (SEQ ID NO:105), GGGSG (SEQ ID NO: 106), and GSSSG (SEQ ID NO:107).

### V. Modifications to Extend Duration of Action In Vivo

The binding proteins described herein can be modified to provide for an extended lifetime in vivo and/or extended duration of action in a subject. In some embodiments, the binding protein can be conjugated to carrier molecules to provide desired pharmacological properties such as an extended half-life. In some embodiments, the binding protein can be covalently linked to the Fc domain of IgG, albumin, or other molecules to extend its half-life, e.g., by pegylation, glycosylation, and the like as known in the art.

In some embodiments, the binding protein is conjugated to a functional domain of an Fc-fusion chimeric polypeptide molecule. Fe fusion conjugates have been shown to increase the systemic half-life of biopharmaceuticals, and thus the biopharmaceutical product can require less frequent administration. Fe binds to the neonatal Fc receptor (FeRn) in endothelial cells that line the blood vessels, and, upon binding, the Fe fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fe region of an antibody to optimize the pharmacokinetic and pharmacodynamic properties of the biopharmaceutical as compared to traditional Fc-fusion conjugates. The “Fc region” useful in the preparation of Fe fusions can be a naturally occurring or synthetic polypeptide that is homologous to an IgG C-terminal domain produced by digestion of IgG with papain. IgG Fe has a molecular weight of approximately 50 kDa. The binding protein described herein can be conjugated to the entire Fc region, or a smaller portion that retains the ability to extend the circulating half-life of a chimeric polypeptide of which it is a part. In addition, full-length or fragmented Fe regions can be variants of the wild-type molecule. In a typical presentation, each monomer of the dimeric Fe can carry a heterologous polypeptide, the heterologous polypeptides being the same or different.

In some embodiments, when the binding protein described herein is to be administered in the format of an Fe fusion, particularly in those situations when the polypeptide chains conjugated to each subunit of the Fc dimer are different, the Fe fusion may be engineered to possess a “knob-into-hole modification.” The knob-into-hole modification is more fully described in Ridgway, et al. (1996) Protein Engineering 9 (7): 617-621 and U.S. Pat. No. 5,731,168, issued Mar. 24, 1998. The knob-into-hole modification refers to a modification at the interface between two immunoglobulin heavy chains in the CH3 domain, wherein: i) in a CH3 domain of a first heavy chain, an amino acid residue is replaced with an amino acid residue having a larger side chain (e.g., tyrosine or tryptophan) creating a projection from the surface (“knob”), and ii) in the CH3 domain of a second heavy chain, an amino acid residue is replaced with an amino acid residue having a smaller side chain (e.g., alanine or threonine), thereby generating a cavity (“hole”) at interface in the second CH3 domain within which the protruding side chain of the first CH3 domain (“knob”) is received by the cavity in the second CH3 domain. In one embodiment, the “knob-into-hole modification” comprises the amino acid substitution T366W and optionally the amino acid substitution S354C in one of the antibody heavy chains, and the amino acid substitutions T366S, L368A, Y407V and optionally Y349C in the other one of the antibody heavy chains. Furthermore, the Fc domains may be modified by the introduction of cysteine residues at positions S354 and Y349 which results in a stabilizing disulfide bridge between the two antibody heavy chains in the Fe region (Carter, et al. (2001) Immunol Methods 248, 7-15). The knob-into-hole format is used to facilitate the expression of a first polypeptide on a first Fc monomer with a “knob” modification and a second polypeptide on the second Fe monomer possessing a “hole” modification to facilitate the expression of heterodimeric polypeptide conjugates.

In some embodiments, the binding protein can be conjugated to one or more water-soluble polymers. Examples of water soluble polymers useful in the practice of the present disclosure include polyethylene glycol (PEG), poly-propylene glycol (PPG), polysaccharides (polyvinylpyrrolidone, copolymers of ethylene glycol and propylene glycol, poly(oxyethylated polyol), polyolefinic alcohol), polysaccharides), poly-alpha-hydroxy acid), polyvinyl alcohol (PVA), polyphosphazene, polyoxazolines (POZ), poly(N-acryloylmorpholine), or a combination thereof.

In some embodiments, binding protein can be conjugated to one or more polyethylene glycol molecules or “PEGylated.” Although the method or site of PEG attachment to the binding protein may vary, in certain embodiments the PEGylation does not alter, or only minimally alters, the activity of the binding protein.

In some instances, when employing the VHH sequences describe herein in the preparation of th IL27 binding molecules of the present disclosure, the VHH possesses an N-terminal glutamine (“1Q”) residue. N-terminal glutamine residues have been observed to spontaneously cyclyize to form pyroglutamate (pE) at or near physiological conditions. (See e.g., Liu, et al (2011) J. Biol. Chem. 286 (13): 11211-11217). In some embodiments, the formation of pyroglutamate complicates N-terminal PEG conjugation particularly when aldehyde chemistry is used for N-terminal PEGylation. Consequently, when PEGylating the IL27R binding molecules of the present disclosure, particularly when aldehyde chemistry is to be employed, the IL27Rα binding molecules possessing an amino acid at position 1 (e.g., 1Q) are substituted at position 1 with an alternative amino acid or are deleted at position 1 (e.g., des-1Q). In some embodiments, the IL27R binding molecules of the present disclosure comprise an amino acid substitution selected from the group Q1E and Q1D.

In some embodiments, selective PEGylation of the binding protein, for example, by the incorporation of non-natural amino acids having side chains to facilitate selective PEG conjugation, may be employed. Specific PEGylation sites can be chosen such that PEGylation of the binding protein does not affect its binding to the target receptors.

In certain embodiments, the increase in half-life is greater than any decrease in biological activity. PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure.

A molecular weight of the PEG used in the present disclosure is not restricted to any particular range. The PEG component of the binding protein can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa, or from about 10 kDa to about 30 kDa. Linear or branched PEG molecules having molecular weights from about 2,000 to about 80,000 daltons, alternatively about 2,000 to about 70,000 daltons, alternatively about 5,000 to about 50,000 daltons, alternatively about 10,000 to about 50,000 daltons, alternatively about 20,000 to about 50,000 daltons, alternatively about 30,000 to about 50,000 daltons, alternatively about 20,000 to about 40,000 daltons, or alternatively about 30,000 to about 40,000 daltons. In one embodiment of the disclosure, the PEG is a 40 kD branched PEG comprising two 20 kD arms.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. Use of a PEG-aldehyde linker targets a single site on the N-terminus of a polypeptide through reductive amination.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General PEGylation strategies known in the art can be applied herein.

The PEG can be bound to a binding protein of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide.

In some embodiments, the PEGylation of the binding proteins is facilitated by the incorporation of non-natural amino acids bearing unique side chains to facilitate site specific PEGylation. The incorporation of non-natural amino acids into polypeptides to provide functional moieties to achieve site specific PEGylation of such polypeptides is known in the art. See e.g., Ptacin et al., PCT International Application No. PCT/US2018/045257 filed Aug. 3, 2018 and published Feb. 7, 2019 as International Publication Number WO 2019/028419A1.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present disclosure include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright®) ME-100AS, Sunbright® ME-100GS, Sunbright® ME-100HS, NOF), a 20 kDa linear PEG-aldehyde (e.g., Sunbright® ME-200AL, NOF), a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 KDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright®) GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright®) GL2-400AL3), a 40 kDa 2-arm branched PEG-NHS ester the 40 kDA PEG-NHS ester comprising two 20 kDA linear PEG molecules (e.g., Sunbright®) GL2-400AL3, Sunbright® GL2-400GS2, NOF), a linear 30 kDa PEG-aldehyde (e.g., Sunbright& ME-300AL) and a linear 30 kDa PEG-NHS ester.

In some embodiments, a linker can used to join the binding protein and the PEG molecule. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as I amino acid (e.g., Gly), 2, 3, 4, 5, 6, 7, 8, 9, 10, 10-20, 20-30, 30-50 or more than 50 amino acids. Examples of flexible linkers are described in Section IV. Further, a multimer (e.g., 1, 2, 3, 4, 5, 6, 7, 8, 9, 10, 10-20, 20-30, or 30-50) of these linker sequences may be linked together to provide flexible linkers that may be used to conjugate two molecules. Alternative to a polypeptide linker, the linker can be a chemical linker, e.g., a PEG-aldehyde linker. In some embodiments, the binding protein is acetylated at the N-terminus by enzymatic reaction with N-terminal acetyltransferase and, for example, acetyl COA. Alternatively, or in addition to N-terminal acetylation, the binding protein can be acetylated at one or more lysine residues, e.g., by enzymatic reaction with a lysine acetyltransferase. See, for example Choudhary et al. (2009) Science 325 (5942): 834-840.

In other embodiments, the binding protein can be modified to include an additional polypeptide sequence that functions as an antigenic tag, such as a FLAG sequence. FLAG sequences are recognized by biotinylated, highly specific, anti-FLAG antibodies, as described herein (see e.g., Blanar et al. (1992) Science 256:1014 and LeClair, et al. (1992) PNAS-USA 89:8145). In some embodiments, the binding protein further comprises a C-terminal c-myc epitope tag.

In some embodiments, the binding protein is expressed as a fusion protein with an albumin molecule (e.g., human serum albumin) which is known in the art to facilitate extended exposure in vivo.

In some embodiment, the binding proteins (including fusion proteins of the binding proteins) of the present disclosure are expressed as a fusion protein with one or more transition metal chelating polypeptide sequences. The incorporation of such a transition metal chelating domain facilitates purification immobilized metal affinity chromatography (IMAC) as described in Smith, et al. U.S. Pat. No. 4,569,794 issued Feb. 11, 1986.

Examples of transition metal chelating polypeptides useful in the practice of the present disclosure are described in Smith, et al. supra and Dobeli, et al. U.S. Pat. No. 5,320,663 issued May 10, 1995, the entire teachings of which are hereby incorporated by reference. Particular transition metal chelating polypeptides useful in the practice of the present disclosure are peptides comprising 3-6 contiguous histidine residues (SEQ ID NO: 1547) such as a six-histidine peptide (His) 6 (SEQ ID NO: 1531) and are frequently referred to in the art as “His-tags.”

The foregoing fusion proteins may be readily produced by recombinant DNA methodology by techniques known in the art by constructing a recombinant vector comprising a nucleic acid sequence comprising a nucleic acid sequence encoding the binding protein in frame with a nucleic acid sequence encoding the fusion partner either at the N-terminus or C-terminus of the binding protein, the sequence optionally further comprising a nucleic acid sequence in frame encoding a linker or spacer polypeptide.

### VI. Pharmaceutical Composition

The binding proteins of the present disclosure may be administered to a subject in a pharmaceutically acceptable dosage form. The preferred formulation depends on the intended mode of administration and therapeutic application. Pharmaceutical dosage forms of the binding proteins described herein comprise physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of polypeptides include polysaccharides such as sodium carboxymethylcellulose or methylcellulose, polyvinylpyrrolidone, polyacrylates, polyoxyethylene-polyoxypropylene-block polymers, PEG, polymeric amino acids, amino acid copolymers, and lipid aggregates (such as oil droplets or liposomes).

The pharmaceutical compositions may also comprise pharmaceutically-acceptable, non-toxic carriers, excipients, stabilizers, or diluents, which are defined as vehicles commonly used to formulate pharmaceutical compositions for animal or human administration. The diluent is selected so as not to affect the biological activity of the combination. Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trebalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Formulations to be used for in vivo administration are typically sterile. Sterilization of the compositions of the present disclosure may readily accomplished by filtration through sterile filtration membranes.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above (Langer, Science 249:1527, 1990 and Hanes, Advanced Drug Delivery Reviews 28:97-119, 1997). The agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Administration of a binding protein described herein may be achieved through any of a variety of art recognized methods including but not limited to the topical, intravascular injection (including intravenous or intraarterial infusion), intradermal injection, subcutaneous injection, intranmiscular injection, intraperitoneal injection, intracranial injection, intratumoral injection, intranodal injection, transdermal, transmucosal, iontophoretic delivery, intralymphatic injection (Senti and Kundig (2009) Current Opinions in Allergy and Clinical Immunology 9 (6): 537-543), intragastric infusion, intraprostatic injection, intravesical infusion (e.g., bladder), respiratory inhalers including nebulizers, intraocular injection, intraabdominal injection, intralesional injection, intraovarian injection, intracerebral infusion or injection, intracerebroventricular injection (ICVI), and the like. In some embodiments, administration includes the administration of the binding protein itself (e.g., parenteral), as well as the administration of a recombinant vector (e.g., viral or non-viral vector) to cause the in situ expression of the binding protein in the subject. Alternatively, a cell, such as a cell isolated from the subject, could also be recombinantly modified to express the binding protein of the present disclosure.

The dosage of the pharmaceutical compositions depends on factors including the route of administration, the disease to be treated, and physical characteristics, e.g., age, weight, general health, of the subject. Typically, the amount of a binding protein contained within a single dose may be an amount that effectively prevents, delays, or treats the disease without inducing significant toxicity. A pharmaceutical composition of the disclosure may include a dosage of a binding protein described herein ranging from 0.01 to 500 mg/kg (e.g., from 0.01 to 450 mg, from 0.01 to 400 mg, from 0.01 to 350 mg, from 0.01 to 300 mg, from 0.01 to 250 mg, from 0.01 to 200 mg, from 0.01 to 150 mg, from 0.01 to 100 mg, from 0.01 to 50 mg, from 0.01 to 10 mg, from 0.01 to 1 mg, from 0.1 to 500 mg/kg, from 1 to 500 mg/kg, from 5 to 500 mg/kg, from 10 to 500 mg/kg, from 50 to 500 mg/kg, from 100 to 500 mg/kg, from 150 to 500 mg/kg, from 200 to 500 mg/kg, from 250 to 500 mg/kg, from 300 to 500 mg/kg, from 350 to 500 mg/kg, from 400 to 500 mg/kg, or from 450 to 500 mg/kg) and, in a more specific embodiment, about 1 to about 100 mg/kg (e.g., about 1 to about 90 mg/kg, about 1 to about 80 mg/kg, about 1 to about 70 mg/kg, about 1 to about 60 mg/kg, about 1 to about 50 mg/kg, about 1 to about 40 mg/kg, about 1 to about 30 mg/kg, about 1 to about 20 mg/kg, about 1 to about 10 mg/kg, about 10 to about 100 mg/kg, about 20 to about 100 mg/kg, about 30 to about 100 mg/kg, about 40 to about 100 mg/kg, about 50 to about 100 mg/kg, about 60 to about 100 mg/kg, about 70 to about 100 mg/kg, about 80 to about 100 mg/kg, or about 90 to about 100 mg/kg). In some embodiments, a pharmaceutical composition of the disclosure may include a dosage of a binding protein described herein ranging from 0.01 to 20 mg/kg (e.g., from 0.01 to 15 mg/kg, from 0.01 to 10 mg/kg, from 0.01 to 8 mg/kg, from 0.01 to 6 mg/kg, from 0.01 to 4 mg/kg, from 0.01 to 2 mg/kg, from 0.01 to 1 mg/kg, from 0.01 to 0.1 mg/kg, from 0.01 to 0.05 mg/kg, from 0.05 to 20 mg/kg, from 0.1 to 20 mg/kg, from 1 to 20 mg/kg, from 2 to 20 mg/kg, from 4 to 20 mg/kg, from 6 to 20 mg/kg, from 8 to 20 mg/kg, from 10 to 20 mg/kg, from 15 to 20 mg/kg). The dosage may be adapted by the physician in accordance with conventional factors such as the extent of the disease and different parameters of the subject.

A pharmaceutical composition containing a binding protein described herein can be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations may decrease as the medical condition improves or increase as the health of the patient declines. A course of therapy may be a single dose or in multiple doses over a period of time. In some embodiments, a single dose is used. In some embodiments, two or more split doses administered over a period of 1, 2, 3, 4, 5, 6, 7, 8, 9, 10, 11, 12, 13, 14, 21, 28, 30, 60, 90, 120 or 180 days are used. Each dose administered in such split dosing protocols may be the same in each administration or may be different. Multi-day dosing protocols over time periods may be provided by the skilled artisan (e.g., physician) monitoring the administration, taking into account the response of the subject to the treatment including adverse effects of the treatment and their modulation as discussed above.

### VII. Indications

### Immune Diseases

The present disclosure further provides methods of treating a subject suffering from a disease disorder or condition by the administration of a therapeutically effective amount of an IL27R binding protein (or nucleic acid encoding an IL27R binding protein including recombinant viruses encoding the IL27R binding protein) of the present disclosure. Disorders amenable to treatment with IL27R binding proteins (including pharmaceutically acceptable formulations comprising IL27R binding proteins and/or the nucleic acid molecules that encode them including recombinant viruses encoding such IL27R binding proteins) of the present disclosure include inflammatory or autoimmune diseases including but not limited to, viral infections (e.g., AIDS, influenza, chronic HCV, chronic viral hepatitis B, C or D), Helicobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes including Type 1 or type 2 diabetes, inflammation, autoimmune disease, atopic diseases, paraneoplastic autoimmune diseases, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome (Seronegativity Enthesopathy Arthropathy Syndrome), juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemie onset rheumatoidarthritis, ankylosing spondylitis, enteropathie arthritis, reactive arthritis, Reiter's syndrome, SEA Syndrome (Seronegativity, Enthesopathy, Arthropathy Syndrome).

Other examples of proliferative and/or differentiative disorders amenable to treatment with IL27R binding proteins (including pharmaceutically acceptable formulations comprising IL27R binding proteins and/or the nucleic acid molecules that encode them including recombinant viruses encoding such IL27R binding proteins) of the present disclosure include, but are not limited to, skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum comeum. In other embodiments, the disorder may involve aberrant activity of a dermal cell, for example, a dermal endothelial, fibroblast, immune cell (e.g., mast cell or macrophage) found in a dermal layer, for example, the papillary layer or the reticular layer.

Examples of skin disorders include psoriasis, psoriatic arthritis, dermatitis (eczema), for example, exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

The compositions of the present disclosure (including pharmaceutically acceptable formulations comprising IL27R binding proteins and/or the nucleic acid molecules that encode them including recombinant viruses encoding such IL27R binding proteins) can also be administered to a patient who is suffering from (or may suffer from) psoriasis or psoriatic disorders. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, plaque psoriasis, moderate to severe plaque psoriasis, psoriasis vulgaris, eruptive psoriasis, psoriatic erythroderma, generalized pustular psoriasis, annular pustular psoriasis, or localized pustular psoriasis.

Combination of IL27R Binding Proteins with Additional Therapeutic Agents for Autoimmune Disease:

The present disclosure provides for the use of the IL27R binding proteins of the present disclosure in combination with one or more additional active agents (“supplementary agents”) in the treatment of autoimmune disease. As used herein, the term “supplementary agents” includes agents that can be administered or introduced separately, for example, formulated separately for separate administration (e.g., as may be provided in a kit) and/or therapies that can be administered or introduced in combination with the IL27R binding proteins.

As used herein, the term “in combination with” when used in reference to the administration of multiple agents to a subject refers to the administration of a first agent at least one additional (i.e., second, third, fourth, fifth, etc.) agent to a subject. For purposes of the present invention, one agent (e.g., IL27R binding protein) is considered to be administered in combination with a second agent (e.g. a therapeutic autoimmune antibody such as Humira®) if the biological effect resulting from the administration of the first agent persists in the subject at the time of administration of the second agent such that the therapeutic effects of the first agent and second agent overlap. For example, the therapeutic antibodies are sometimes administered by IV infusion every two weeks (e.g. adalimumab in the treatment of Crohn's disease) while the IL27R binding proteins of the present disclosure may be administered more frequently, e.g. daily, BID, or weekly. However, the administration of the first agent (e.g. entaercept) provides a therapeutic effect over an extended time and the administration of the second agent (e.g. an IL27R binding protein) provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. In one embodiment, one agent is considered to be administered in combination with a second agent if the first and second agents are administered simultaneously (within 30 minutes of each other), contemporaneously or sequentially. In some embodiments, a first agent is deemed to be administered “contemporaneously” with a second agent if first and second agents are administered within about 24 hours of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation is administered to a subject. In certain embodiments, the IL27R binding protein and the supplementary agent(s) are administered or applied sequentially, e.g., where one agent is administered prior to one or more other agents. In other embodiments, the IL27R binding protein and the supplementary agent(s) are administered simultaneously, e.g., where two or more agents are administered at or about the same time: the two or more agents may be present in two or more separate formulations or combined into a single formulation (i.e., a co-formulation). Regardless of whether the agents are administered sequentially or simultaneously, they are considered to be administered in combination for purposes of the present disclosure.

In some embodiments, the supplementary agent is one or more agents selected from the group consisting of corticosteroids (including but not limited to prednisone, budesonide, prednilisone), Janus kinase inhibitors (including but not limited to tofacitinib (Xeljanz®), calcineurin inhibitors (including but not limited to cyclosporine and tacrolimus), mTor inhibitors (including but not limited to sirolimus and everolimus), IMDH inhibitors (including but not limited to azathioprine, leflunomide and mycophenolate), biologics such as abatcept (Orencia®) or etanercept (Enbrel®), and therapeutic antibodies. Examples of therapeutic antibodies that may be administered as supplementary agents in combination with the IL27R binding proteins of the present disclosure in the treatment of autoimmune disease include but are not limited to anti-CD25 antibodies (e.g. daclizumab and basiliximab), anti-VLA-4 antibodies (e.g. natalizumab), anti-CD52 antibodies (e.g. alemtuzumab), anti-CD20 antibodies (e.g. rituximab, ocrelizumab), anti-TNF antibodies (e.g. infliximab, and adalimumab), anti-IL6R antibodies (e.g. tocilizumab), anti-TNFα antibodies (e.g. adalimumab (Humira®), golimumab, and infliximab), anti-integrin-α4β7 antibodies (e.g. vedolizumab), anti-IL17a antibodies (e.g. brodalumab or secukinumab), anti-ILARα antibodies (e.g. dupilumab), anti-RANKL antibodies, IL6R antibodies, anti-IL18 antibodies (e.g. canakinumab), anti-CD11a antibodies (e.g. efalizumab), anti-CD3 antibodies (e.g. muramonab), anti-IL5 antibodies (e.g. mepolizumab, reslizumab), anti-BLyS antibodies (e.g. belimumab); and anti-IL12/IL23 antibodies (e.g ustekinumab).

Many therapeutic antibodies have been approved for clinical use against autoimmune disease. Examples of antibodies approved by the United States Food and Drug Administration (FDA) for use in the treatment of autoimmune diseases in a subject suffering therefrom that may be administered as supplementary agents in combination with the IL27R binding proteins of the present disclosure (and optionally additional supplementary agents) for the treatment of the indicated autoimmune disease are provided in Table 4.

TABLE 4

| Name | Target | Indication |
|---|---|---|
| belimumab | BLyS | Systemic lupus erythematosus |
| efalizumab | CD11a | Psoriasis |
| ocrelizumab | CD20 | Multiple sclerosis |
| rituximab | CD20 | Multiple sclerosis |
| basiliximab | CD25 | Transplantation rejection |
| daclizumab | CD25 | Transplantation rejection |
| muromonab | CD3 | Transplantation rejection |
| alemtuzumab | CD52 | Multiple sclerosis |
| omalizumab | IgE | Asthma |
| ustekinumab | IL12/IL23 | Plaque psoriasis |
| brodalumab | IL17a | Psoriasis, psoriatic arthritis, ankylosing spondylitis |
| secukinumab | IL17a | Psoriasis, psoriatic arthritis, ankylosing spondylitis |
| ixekizumab | IL17a | Psoriasis, psoriatic arthritis, ankylosing spondylitis |
| canakinumab | IL1β | Cryopyrin-associated periodic syndrome, tumor necrosis factor |
| | | receptor associated periodic syndrome, hyperimmunoglobulin D |
| | | syndrome, mevalonate kinase deficiency, familial Mediterranean fever, |
| | | rheumatoid arthritis |
| dupilumab | IL4Rα | Asthma, dermatitis |
| mepolizumab | IL5 | Asthma |
| reslizumab | IL5 | Asthma |
| tocilizumab | IL6R | Rheumatoid arthritis |
| vedolizumab | Integrin-α4β7 | Ulcerative colitis, Crohn's disease |
| denosumab | RANKL | Osteoporosis |
| certolizumab | TNFa | Chron's disease, rheumatoid arthritis |
| golimumab | TNFa | Rheumatoid arthritis, psoriatic arthritis, ankylosing spondylitis |
| adalimumab | TNFα | Rheumatoid arthritis, juvenile idiopathic arthritis, psoriatic arthritis, |
| | | ankylosing spondylitis, Crohn's disease, plaque psoriasis |
| infliximab | TNFα | Crohn's disease, ulcerative colitis, rheumatoid arthritis, ankylosing |
| | | spondylitis, psoriatic arthritis, plaque psoriasis |
| ranibizumab | VEGF-A | Neovascular age-related macular degeneration, macular edema |
| natalizumab | VLA-4 | Multiple sclerosis, relapsing rultiple sclerosis, Crohn's disease |

### Treatment of Neoplastic Disease

The present disclosure provides methods of use of IL-27R binding molecules in the treatment of subjects suffering from a neoplastic disease disorder or condition by the administration of a therapeutically effective amount of a IL-27R binding molecule (or nucleic acid encoding a IL-27R binding molecule including recombinant vectors encoding IL-27R binding molecules, and eucaryotic and procaryotic cells modified to express a IL-27R binding molecule) as described herein.

### Neoplasms Amenable to Treatment:

The compositions and methods of the present disclosure are useful in the treatment of subject suffering from a neoplastic disease characterized by the presence neoplasmis, including benign and malignant neoplasms, and neoplastic disease.

Examples of benign neoplasms amenable to treatment using the compositions and methods of the present disclosure include but are not limited to adenomas, fibromas, hemangiomas, and lipomas. Examples of pre-malignant neoplasms amenable to treatment using the compositions and methods of the present disclosure include but are not limited to hyperplasia, atypia, metaplasia, and dysplasia. Examples of malignant neoplasms amenable to treatment using the compositions and methods of the present disclosure include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including but not limited to breast cancers; sarcomas (including but not limited to osteosarcomas and angiosarcomas and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ: intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis.

The term neoplastic disease includes carcinomas. The term “carcinoma” refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The term neoplastic disease includes adenocarcinomas. An “adenocarcinoma” refers to a carcinoma derived from glandular tissue or in which the tumor cells form recognizable glandular structures.

As used herein, the term “hematopoietic neoplastic disorders” refers to neoplastic diseases involving hyperplastic/neoplastic cells of hematopoietic origin, e.g., arising from myeloid, lymphoid or erythroid lineages, or precursor cells thereof.

Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Exemplary myeloid disorders amenable to treatment in accordance with the present disclosure include, but are not limited to, acute promyeloid leukemia (APML), acute myelogenous leukemia (AML) and chronic myelogenous leukemia (CML).

Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Exemplary lymphic disorders amenable to treatment in accordance with the present disclosure include, but are not limited to, acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

In some instances, the hematopoietic neoplastic disorder arises from poorly differentiated acute leukemias (e.g., erythroblastic leukemia and acute megakaryoblastic leukemia). As used herein, the term “hematopoietic neoplastic disorders” refers malignant lymphomas including, but are not limited to, non-Hodgkins lymphoma and variants thereof, peripheral T cell lymphomas, adult T-cell leukemia/lymphoma (ATL), cutaneous T cell lymphoma (CTCL), large granular lymphocytic leukemia (LGF), Hodgkin's disease and Reed-Stemberg disease.

The determination of whether a subject is “suffering from a neoplastic disease” refers to a determination made by a physician with respect to a subject based on the available information accepted in the field for the identification of a disease, disorder or condition including but not limited to X-ray, CT-scans, conventional laboratory diagnostic tests (e.g. blood count, etc.), genomic data, protein expression data, immunohistochemistry, that the subject requires or will benefit from treatment.

### Assessing Anti-Neoplastic Efficacy:

The determination of efficacy of the methods of the present disclosure in the treatment of cancer is generally associated with the achievement of one or more art recognized parameters such as reduction in lesions particularly reduction of metastatic lesion, reduction in metastasis, reduction in tumor volume, improvement in ECOG score, and the like. Determining response to treatment can be assessed through the measurement of biomarker that can provide reproducible information useful in any aspect of IL-27R binding molecule therapy, including the existence and extent of a subject's response to such therapy and the existence and extent of untoward effects caused by such therapy. By way of example, but not limitation, biomarkers include enhancement of IFNγ, and upregulation of granzyme A, granzyme B, and perforin; increase in CD8+ T-cell number and function; enhancement of IFNγ, an increase in ICOS expression on CD8+ T-cells, enhancement of IL-10 expressing T_{Reg} cells. The response to treatment may be characterized by improvements in conventional measures of clinical efficacy may be employed such as Complete Response (CR), Partial Response (PR), Stable Disease (SD) and with respect to target lesions, Complete Response (CR),” Incomplete Response/Stable Disease (SD) as defined by RECIST as well as immune-related Complete Response (irCR), immune-related Partial Response (irPR), and immune-related Stable Disease (irSD) as defined Immune-Related Response Criteria (irRC) are considered by those of skill in the art as evidencing efficacy in the treatment of neoplastic disease in mammalian (e.g. human) subjects.

### Maintenance of Serum Concentration:

In some embodiments of the invention the present disclosure provides methods and compositions for the treatment and/or prevention of neoplastic diseases, disorders or conditions by the administration of a therapeutically effective amount of an IL-27R binding molecules the serum concentration of the IL-27R binding molecule is maintained for a majority (i.e., greater than about 50% of the period of time, alternatively greater than about 60%, alternatively greater than about 70%, alternatively greater than about 80%, alternatively greater than about 90%) of a period of time (e.g. at least 24 hours, alternatively at least 48 hours, alternatively at least 72 hours, alternatively at least 96 hours, alternatively at least 120 hours, alternatively at least 144 hours, alternatively at least 7 days, alternatively at least 10 days, alternatively at least 12 days, alternatively at least 14 days, alternatively at least 28 days, alternatively at least 45 days, alternatively at least 60 days, or longer) at a serum concentration at or above the therapeutically effective concentration with respect to such IL-27R binding molecule.

Combination of IL-27R Binding Molecules with Supplementary Therapeutic Agents:

The present disclosure provides for the use of the IL-27R binding molecules of the present disclosure in combination with one or more additional active agents (“supplementary agents”). Such further combinations are referred to interchangeably as “supplementary combinations” or “supplementary combination therapy” and those therapeutic agents that are used in combination with IL-27R binding molecules of the present disclosure are referred to as “supplementary agents.” As used herein, the term “supplementary agents” includes agents that can be administered or introduced separately, for example, formulated separately for separate administration (e.g., as may be provided in a kit) and/or therapies that can be administered or introduced in combination with the IL-27R binding molecules.

### Chemotherapeutic Agents:

In some embodiments, the supplementary agent is a chemotherapeutic agent. In some embodiments the supplementary agent is a “cocktail” of multiple chemotherapeutic agents. IN some embodiments the chemotherapeutic agent or cocktail is administered in combination with one or more physical methods (e.g. radiation therapy). The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethyl olomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins such as bleomycin A₂., cactinomycin, calicheamicin, carabicin, caminomycin, carzinophilin, chromomycins, dactinomycin, daunorubicin and derivaties such as demethoxy-daunomycin, 11-deoxydaunorubicin, 13-deoxydaunorubicin, detorubicin, 6-diazo-5-oxo-L-norleucine, doxorubicin, epirubicin, esorubicin, idarubicin, marcellomycin, mitomycins such as mitomycin C. N-methyl mitomycin C; mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, dideazatetrahydrofolic acid, and folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; best rabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C): cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylomithine (DMFO); retinoic acid; esperamicins; capecitabine; taxanes such as paclitaxel, docetaxel, cabazitaxel; carminomycin, adriamycins such as 4′-epiadriamycin, 4-adriamycin-14-benzoate, adriamycin-14-octanoate, adriamycin-14-naphthaleneacetate; cholchicine and pharmaceutically acceptable salts, acids or derivatives of any of the above.

The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above.

In some embodiments, a supplementary agent isone or more chemical or biological agents identified in the art as useful in the treatment of neoplastic disease, including, but not limited to, a cytokines or cytokine antagonists such as IL-12, INFα, or anti-epidermal growth factor receptor, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec®) or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In some embodiments, the IL-27R binding molecule is administered in combination with BRAF/MEK inhibitors, kinase inhibitors such as sunitinib, PARP inhibitors such as olaparib, EGFR inhibitors such as osimertinib (Ahn, et al. (2016) J Thorac Oncol 11: S115), IDO inhibitors such as epacadostat, and oncolytic viruses such as talimogene laherparepvec (T-VEC).

### Anti-Tumor Antigen Antibody Therapeutics as Supplementary Agents

In some embodiments, a “supplementary agent” is a therapeutic antibody (including bi-specific and tri-specific antibodies which bind to one or more tumor associated antigens including but not limited to bispecific T cell engagers (BITEs), dual affinity retargeting (DART) constructs, and trispecific killer engager (TrikE) constructs).

In some embodiments, the therapeutic antibody is an antibody that binds to at least one tumor antigen selected from the group consisting of HER2 (e.g. trastuzumab, pertuzumab, ado-trastuzumab emtansine), nectin-4 (e.g. enfortumab), CD79 (e.g. polatuzumab vedotin), CTLA4 (e.g. ipilumumab), CD22 (e.g. moxetumomab pasudotox), CCR4 (e.g. magamuizumab), IL23p19 (e.g. tildrakizumab), PDL1 (e.g. durvalumab, avelumab, atezolizumab), IL17a (e.g. ixekizumab), CD38 (e.g. daratumumab), SLAMF7 (e.g. elotuzumab), CD20 (e.g. rituximab, tositumomab, ibritumomab and ofatumumab), CD30 (e.g. brentuximab vedotin), CD33 (e.g. gemtuzumab ozogamicin), CD52 (e.g. alemtuzumab), EpCam, CEA, fpA33, TAG-72, CAIX, PSMA, PSA, folate binding protein, GD2 (e.g. dinuntuximab), GD3, IL6 (e.g. silutxumab) GM2, Ley, VEGF (e.g. bevacizumab), VEGFR, VEGFR2 (e.g. ramucirumab), PDGFRα (e.g. olartumumab), EGFR (e.g. cetuximab, panitumumab and necitumumab), ERBB2 (e.g. trastuzumab), ERBB3, MET, IGF1R, EPHA3, TRAIL R1, TRAIL R2, RANKL RAP, tenascin, integrin αVβ3, and integrin α4β1.

Examples of antibody therapeutics which are FDA approved and may be used as supplementary agents for use in the treatment of neoplastic disease include those provided in the Table below.

TABLE
Approved Antineoplastic Disease Antibodies and Indications

| Name | Tradename(s) | Target; format | Indication |
|---|---|---|---|
| [fam]-trastuzumab | Enhertu ™ | HER2; Humanized IgG1 ADC | HER2+ breast cancer |
| deruxtecan | | | |
| Enfortumab vedotin | Padcev ™ | Nectin-4; Human IgG1 ADC | Urothelial cancer |
| Polatuzumab vedotin | Polivy ™ | CD79b; Humanized IgG1 ADC | Diffuse large B-cell lymphoma |
| Cemiplimab | Libtayo ™ | PD-1; Human mAb | Cutaneous squamous cell |
| | | | carcinoma |
| Moxetumomab pasudotox | Lumoxiti ™ | CD22; Murine | Hairy cell leukemia |
| | | IgG1 dsFv immunotoxin | |
| Mogamuizumab | Poteligeo ™ | CCR4; Humanized IgG1 | Cutaneous T cell lymphoma |
| Tildrakizumab | Ilumya ™ | IL23p19; Humanized IgG1 | Plaque psoriasis |
| Ibalizumab | Trogarzo ™ | CD4; Humanized IgG4 | HIV infection |
| Durvalumab | IMFINZI ™ | PD-L1; Human IgG1 | Bladder cancer |
| Inotuzumab | BESPONSA ™ | CD22; Humanized IgG4, ADC | Hematological malignancy |
| ozogamicin | | | |
| Avelumab | Bavencio ™ | PD-L1; Human IgG1 | Merkel cell carcinoma |
| Atezolizumab | Tecentriq ™ | PD-L1; Humanized IgG1 | Bladder cancer |
| Olaratumab | Lartruvo ™ | PDGRFα; Human IgG1 | Soft tissue sarcoma |
| Ixekizumab | Taltz ™ | IL-17a; Humanized IgG4 | Psoriasis |
| Daratumunab | Darzalex ™ | CD38; Human IgG1 | Multiple myeloma |
| Elotuzumab | Empliciti ™ | SLAMF7; Humanized IgG1 | Multiple myeloma |
| Necitumumab | Portrazza ™ | EGFR; Human IgG1 | Non-small cell lung cancer |
| Dinutuximab | Unituxin ™ | GD2; Chimeric IgG1 | Neuroblastoma |
| Nivolumab | Opdivo ™ | PD1; Human IgG4 | Melanoma, non-small cell lung |
| | | | cancer |
| Blinatumomab | Blincyto ™ | CD19, CD3; Murine bispecific | Acute lymphoblastic leukemia |
| | | tandem scFv | |
| Pembrolizumab | Keytruda ™ | PD1; Humanized IgG4 | Melanoma |
| Ramucirumab | Cyramza ™ | VEGFR2; Human IgG1 | Gastric cancer |
| Siltuximab | Sylvant ™ | IL-6; Chimeric IgG1 | Castleman disease |
| Obinutuzumab | Gazyva ™ | CD20; Humanized IgG1; | Chronic lymphocytic leukemia |
| | | Glycoengineered | |
| Ado-trastuzumab | Kadcyla ™ | HER2; Humanized IgG1, ADC | Breast cancer |
| emtansine | | | |
| Pertuzumab | Perjeta ™ | HER2; Humanized IgG1 | Breast Cancer |
| Brentuximab vedotin | Adcetris ™ | CD30; Chimeric IgG1, ADC | Hodgkin lymphoma, systemic |
| | | | anaplastic large cell lymphoma |
| Ipilimumab | Yervoy ™ | CTLA-4; Human IgG1 | Metastatic melanoma |
| Ofatumumab | Arzerra ™ | CD20; Human IgG1 | Chronic lymphocytic leukemia |
| Certolizumab pegol | Cimzia ™ | TNF; Humanized Fab, | Crohn disease |
| | | pegylated | |
| Catumaxomab | Removab ™ | EPCAM/CD3; Rat/mouse | Malignant ascites |
| | | bispecific mAb | |
| Panitumumab | Vectibix ™ | EGFR; Human IgG2 | Colorectal cancer |
| Bevacizumab | Avastin ™ | VEGF; Humanized IgG1 | Colorectal cancer |
| Cetuximab | Erbitux ™ | EGFR; Chimeric IgG1 | Colorectal cancer |
| Tositumomab-I131 | Bexxar ™ | CD20; Murine IgG2a | Non-Hodgkin lymphoma |
| Ibritumomab tiuxetan | Zevalin ™ | CD20; Murine IgG1 | Non-Hodgkin lymphoma |
| Gemtuzumab | Mylotarg ™ | CD33; Humanized IgG4, ADC | Acute myeloid leukemia |
| ozogamicin | | | |
| Trastuzumab | Herceptin ™ | HER2; Humanized IgG1 | Breast cancer |
| Infliximab | Remicade ™ | TNF; Chimeric IgG1 | Crohn disease |
| Rituximab | MabThera ™, | CD20; Chimeric IgG1 | Non-Hodgkin lymphoma |
| | Rituxan ™ | | |
| Edrecolomab | Panorex ™ | EpCAM; Murine IgG2a | Colorectal cancer |

### Physical Methods

In some embodiments, a supplementary agent is one or more non-pharmacological modalities (e.g., localized radiation therapy or total body radiation therapy or surgery). By way of example, the present disclosure contemplates treatment regimens wherein a radiation phase is preceded or followed by treatment with a treatment regimen comprising a IL-27R binding molecule and one or more supplementary agents. In some embodiments, the present disclosure further contemplates the use of a IL-27R binding molecule in combination with surgery (e.g. tumor resection). In some embodiments, the present disclosure further contemplates the use of a IL-27R binding molecule in combination with bone marrow transplantation, peripheral blood stem cell transplantation or other types of transplantation therapy.

Combination with Immune Checkpoint Modulators:

In some embodiments, a “supplementary agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

As used herein, the term “immune checkpoint pathway modulator” refers to a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. The term “immune checkpoint pathway modulators” encompasses both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up-regulating at least partially the function of a stimulatory immune checkpoint (referred to herein as an “immune checkpoint pathway effector” or “immune checkpoint pathway agonist.”).

Immune checkpoint modulators include but are not limited to immune checkpoint antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H5) receptor, LAG3 (lymphocyte activation gene 3; also known as CD233) and CTLA4 (cytotoxic T-lymphocyte associated antigen 4; also known as CD152). In some embodiments, immune checkpoint modulators are agonists that trigger the checkpoint pathway resulting stimulation of the immune response. Examples of such agonist immune checkpoint modulators include, but is not limited to, agonist that modulate the binding of ICOSL to ICOS(CD278), B7-H6 to NKp30, CD155 to CD96, OX40L to OX40, CD70 to CD27, CD40 to CD40L, and GITRL to GITR. Examples of such positive immune checkpoint agonists include but are not limited to agonist antibodies that bind T-cell activating receptors such as ICOS (such as JTX-2011, Jounce Therapeutics), OX40 (such as MEDI6383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN1876, Agenus).

Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor). The term PD1 pathway inhibitors includes monoclonal antibodies that interfere with the binding of PD1 to PDL1 and/or PDL2. Examples of commercially available PD1 pathway inhibitors useful as supplementary agents in the treatment of neoplastic disease include antibodies that interfere with the binding of PD1 to PDL1 and/or PDL2 including but not limited to nivolumab (Opdivo®, BMS-936558, MDX1106, commercially available from BristolMyers Squibb, Princeton NJ), pembrolizumab (Keytruda®) MK-3475, lambrolizumab, commercially available from Merck and Company, Kenilworth NJ), and atezolizumab (Tecentriq®), Genentech/Roche, South San Francisco CA), Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

The term PD1 pathway inhibitors are not limited to antagonist antibodies. Non-antibody biologies PD1 pathway inhibitors are also under clinical development including AMP-224, a PD-L2 IgG2a fusion protein, and AMP-514, a PDL2 fusion protein, are under clinical development by Amplimmune and Glaxo SmithKline), aptamers (Wang, et al. (2018) 145:125-130), peptide PD1 pathway inhibitors (Sasikumar, et al., U.S. Pat. No. 9,422,339 issued Aug. 23, 2016, and Sasilkumar, et al., U.S. Pat. No. 8,907,053 issued Dec. 9, 2014), small molecules (CA-170, AUPM-170, Aurigene/Curis; Sasikumar, et al., 1,2,4-oxadiazole and thiadiazole compounds as immunomodulators (PCT/IB2016/051266 filed Mar. 7, 2016, published as WO2016142833A1 Sep. 15, 2016) and Sasikumar, et al PCT/IB2016/051343 filed Mar. 9, 2016 and published as WO2016142886A2), BMS-1166 and Chupak L S and Zheng X. (2015) WO 2015/034820 A1, EP3041822 B1 granted Aug. 9, 2017; WO2015034820 A1; and Chupak, et al. 2015) WO 2015/160641 A2. WO 2015/160641 A2, Chupak, et al. Sharpe, et al. WO 2011082400 A2 published Jul. 7, 2011; U.S. Pat. No. 7,488,802 issued Feb. 10, 2009;

In some embodiments, the IL-27R binding molecule is administered in combination with an antagonist of a negative immune checkpoint pathway that inhibits the binding of CTLA4 to CD28 (“CTLA4 pathway inhibitor”). Examples of CTLA4 pathway inhibitors are well known in the art (See, e.g., U.S. Pat. No. 6,682,736 (Abgenix) issued Jan. 27, 2004; U.S. Pat. No. 6,984,720 (Medarex, Inc.) issued May 29, 2007; U.S. Pat. No. 7,605,238 (Medarex, Inc.) issued Oct. 20, 2009)

In some embodiments, the IL-27R binding molecule is administered in combination with an antagonist of a negative immune checkpoint pathway that inhibits the ability TIM3 to binding to TIM3-activating ligands (“TIM3 pathway inhibitor”). Examples of TIM3 pathway inhibitors are known in the art and with representative non-limiting examples described in PCT International Patent Publication No. WO 2016/144803 published Sep. 15, 2016; Lifke, et al. United States Patent Publication No. US20160257749 A1 published Sep. 8, 2016 (F. Hoffman-LaRoche); Karunsky, U.S. Pat. No. 9,631,026 issued Apr. 27, 2017; Karunsky, Sabatos-Peyton, et al. U.S. Pat. No. 8,841,418 issued Sep. 23, 2014; U.S. Pat. No. 9,605,070; Takayanagi, et al., U.S. Pat. No. 8,552,156 issued Oct. 8, 2013.

In some embodiments, the IL-27R binding molecule is administered in combination with an inhibitor of both LAG3 and PD1 as the blockade of LAG3 and PD1 has been suggested to synergistically reverse anergy among tumor-specific CD8+ T-cells and virus-specific CD8+ T-cells in the setting of chronic infection. IMP321 (ImmmuFact) is being evaluated in melanoma, breast cancer, and renal cell carcinoma. See generally Woo et al., (2012) Cancer Res 72:917-27; Goldberg et al., (2011) Curr. Top. Microbiol. Inmunol. 344:269-78; Pardoll (2012) Nature Rev. Cancer 12:252-64; Grosso et al., (2007) J. Clin. Invest. 117:3383-392.

In some embodiments, the IL-27R binding molecule is administered in combination with an A2aR inhibitor. A2aR inhibits T-cell responses by stimulating CD4+ T-cells towards developing into T_{Reg} cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by the administration of molecules such as antibodies that block adenosine binding or by adenosine analogs. Such agents may be used in combination with the IL-27R binding molecules for use in the treatment disorders such as cancer and Parkinson's disease.

In some embodiments, the IL-27R binding molecule is administered in combination with an inhibitor of IDO (Indoleamine 2,3-dioxygenase). IDO down-regulates the immune response mediated through oxidation of tryptophan resulting in in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

As previously described, the present invention provides for a method of treatment of neoplastic disease (e.g. cancer) in a mammalian subject by the administration of a IL-27R binding molecule in combination with an agent(s) that modulate at least one immune checkpoint pathway including immune checkpoint pathway modulators that modulate two, three or more immune checkpoint pathways.

In some embodiments the IL-27R binding molecule is administered in combination with an immune checkpoint modulator that modulates multiple immune checkpoint pathways. Multiple immune checkpoint pathways may be modulated by the administration of multi-functional molecules which act as modulators of multiple immune checkpoint pathways. Examples of such multiple immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of IL-27R binding molecules in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

In some embodiments, the IL-27R binding molecule may be administered in combination with two, three, four or more checkpoint pathway modulators. Such combinations may be advantageous in that immune checkpoint pathways may have distinct mechanisms of action, which provides the opportunity to attack the underlying disease, disorder or conditions from multiple distinct therapeutic angles.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. Therefore, a determination as to whether treatment with an immune checkpoint pathway inhibitors(s) in combination with a IL-27R binding molecule of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

### Cell Therapy Agents and Methods as Supplementary Agents:

In some embodiments, the methods of the disclosure may include the combination of the administration of a IL-27R binding molecules with supplementary agents in the form of cell therapies for the treatment of neoplastic, autoimmune or inflammatory diseases. Examples of cell therapies that are amenable to use in combination with the methods of the present disclosure include but are not limited to engineered T cell products comprising one or more activated CAR-T cells, engineered TCR cells, tumor infiltrating lymphocytes (TILs), engineered Treg cells. As engineered T-cell products are commonly activated ex vivo prior to their administration to the subject and therefore provide upregulated levels of CD25, cell products comprising such activated engineered T cells types are amenable to further support via the administration of an CD25 biased IL-27R binding molecule as described herein.

In some embodiments of the methods of the present disclosure, the supplementary agent is a “chimeric antigen receptor T-cell” and “CAR-T cell” are used interchangeably to refer to a T-cell that has been recombinantly modified to express a chimeric antigen receptor. As used herein, the terms As used herein, the terms “chimeric antigen receptor” and “CAR” are used interchangeably to refer to a chimeric polypeptide comprising multiple functional domains arranged from amino to carboxy terminus in the sequence: (a) an antigen binding domain (ABD), (b) a transmembrane domain (TD); and (c) one or more cytoplasmic signaling domains (CSDs) wherein the foregoing domains may optionally be linked by one or more spacer domains. The CAR may also further comprise a signal peptide sequence which is conventionally removed during post-translational processing and presentation of the CAR on the cell surface of a cell transformed with an expression vector comprising a nucleic acid sequence encoding the CAR. CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3 (4): 388-398; Jensen and Riddell (2015) Current Opinions in Immunology 33:9-15; Gross, et al. (1989) PNAS (USA) 86 (24): 10024-10028; Curran, et al. (2012) J Gene Med 14 (6): 405-15. Examples of commercially available CAR-T cell products that may be modified to incorporate an orthogonal of receptor the present invention include axicabtagene ciloleucel (marketed as Yescarta® commercially available from Gilead Pharmaceuticals) and tisagenlecleucel (marketed as Kymriah® commercially available from Novartis). In some embodiments, the CAR-T possesses a CAR specifically binds to a cell surface molecule associated with a tumor cell is selected from the group consisting of GD2. BCMA, CD19, CD33, CD38, CD70, GD2, IL3Rα2, CD19, mesothelin, Her2, EpCam, Muc1, ROR1, CD133, CEA, EGRFRVIII, PSCA, GPC3, Pan-ErbB and FAP

### Physical Methods:

In some embodiments, the supplementary agent is a anti-neoplastic physical methods including but not limited to radiotherapy, cryotherapy, hyperthermic therapy, surgery, laser ablation, and proton therapy.

Kits: The present disclosure also contemplates kits comprising pharmaceutical compositions IL-27R binding molecules and a pharmaceutical composition thereof. The kits are generally in the form of a physical structure housing various components, as described below, and can be utilized, for example, in practicing the methods described above. A kit may comprise a IL-27R binding molecule in the form of a pharmaceutical composition suitable for administration to a subject that is ready for use or in a form or requiring preparation for example, thawing, reconstitution or dilution prior to administration. When the IL-27R binding molecule is in a form that needs to be reconstituted by a user, the kit may also comprise a sterile container providing a reconstitution medium comprising buffers, pharmaceutically acceptable excipients, and the like. A kit of the present disclosure can be designed for conditions necessary to properly maintain the components housed therein (e.g., refrigeration or freezing). A kit may further contain a label or packaging insert including identifying information for the components therein and instructions for their use. Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial). Labels or inserts may be provided in a physical form or a computer readable medium. In some embodiments, the actual instructions are not present in the kit, but rather the kit provides a means for obtaining the instructions from a remote source, e.g., via an internet site, including by secure access by providing a password (or scannable code such as a barcode or QR code on the container of the IL-27R binding molecule or kit comprising) in compliance with governmental regulations (e.g., HIPAA) are provided.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

No admission is made that any reference cited herein constitutes prior art. The discussion of the references states what their authors assert, and the inventors reserve the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The foregoing antibodies useful as supplementary agents in the practice of the methods of the present disclosure may be administered alone or in the form of any antibody drug conjugate (ADC) comprising the antibody, linker, and one or more drugs (e.g. 1, 2, 3, 4, 5, 6, 7, or 8 drugs) or in modified form (e.g. PEGylated).

In some embodiments the supplementary agent is a vaccine. The IL27R binding proteins of the present invention may be administered to a subject in combination with vaccines as an adjuvant to enhance the immune response to the vaccine in accordance with the teaching of Doyle, et al Unite States U.S. Pat. No. 5,800,819 issued Sep. 1, 1998. Examples of vaccines that may be combined with the IL27R binding proteins of the present invention include are HSV vaccines, Bordetella pertussis, Escherichia coli vaccines, pneumococcal vaccines including multivalent pneumococcal vaccines such as Prevnar® 13, diptheria, tetanus and pertussis vaccines (including combination vaccines such as Pediatrix®) and Pentacel®), varicella vaccines, Haemophilus influenzae type B vaccines, human papilloma virus vaccines such as Garasil®, polio vaccines, Leptospirosis vaccines, combination respiratory vaccine, Moraxella vaccines, and attenuated live or killed virus vaccine products such as bovine respiratory disease vaccine (RSV), multivalent human influenza vaccines such as Fluzone® and Quadravlent Fluzone®), feline leukemia vaccine, transmissible gastroenteritis vaccine, COVID-19 vaccine, and rabies vaccine.

For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

### EXAMPLES

### Example 1

Camels were acclimated at research facility for at least 7 days before immunization. Antigen was diluted with 1×PBS (antigen total about 1 mg). The quality of the antigen was assessed by SDS-PAGE to ensure purity (e.g., >80%). For the first time, 10 mL CFA (then followed 6 times using IFA) was added into mortar, then 10 mL antigen in 1×PBS was slowly added into the mortar with the pestle grinding. The antigen and CFA/IFA were ground until the component showed milky white color and appeared hard to disperse. Camels were injected with antigen emulsified in CFA subcutaneously at at least six sites on the body, injecting about 2 mL at each site (total of 10 mL per camel). A stronger immune response was generated by injecting more sites and in larger volumes. The immunization was conducted every week (7 days), for 7 times. The needle was inserted into the subcutaneous space for 10 to 15 seconds after each injection to avoid leakage of the emulsion. Alternatively, a light pull on the syringe plunger also prevented leakage. The blood sample was collected three days later after 7^{th} immunization.

100 mL of blood was collected from the camel three days following the last injection in the immunization protocol. RNA was extracted from blood and transcribed to cDNA. The approximately 900 bp reverse transcribed sequences encoding the VH-CH1-hinge-CH2-CH3 constructs were isolated from the approximately desired 700 bp fragments encoding the VHH-hinge-CH2-CH3 species. The purified approximately 700 bp fragments were amplified by nested PCR. The amplified sequences were digested using Patland Not1. The approximately 400 bp PST1/Not1 digested fragments were inserted into a Pst1/Not1 digested pMECS phagemid vector such that the sequence encoding the VHH was in frame with a DNA sequence encoding a HA/His sequence. The PCR generated sequences and the vector of pMECS phagemid were digested with Pst I and Not I, subsequently, ligated to pMECS/Nb recombinant. After ligation, the products were transformed into Escherichia coli (E. coli) TG1 cells by electroporation. The transformants were enriched in growth medium, followed by transfer to 2YT+2% glucose agar plates.

Bio-panning of the phage library was conducted to identify VHHs that bind IL27Rα. A 96-well plate was coated with IL27Rα and the phage library was incubated in each well to allow phage-expressing IL27Rα reactive VHH to bind to the IL27Rα on the plate. Non-specifically bound phage were washed off and the specifically bound phage isolated. After the selection, the enriched phage library expressing IL27Rα reactive VHH were amplified in TG1 cells. The aforementioned bio-panning process was repeated for 2-3 rounds to enrich the library for VHH selective for IL27Rα. Once biopanning was complete, three 96-well plates of individual phage clones were isolated in order to perform periplasmic extract ELISA PE-ELISA) on antigen coated plates to identify positive VHH binders. Briefly, A 96-well plate was coated with antigen and PBS under the same conditions. Next, wells were blocked at 37° C. for 1 h. Then, 100 μl of extracted antibodies was added to each well and incubated for 1 h. Subsequently, 100 μl of anti-tag polyclonal antibody conjugated to HRP was added to each well and incubated at 37° C. for 1 h. Plates were developed with TMB substrate. The reaction was stopped by the addition of H2SO4. Absorbance at 450 mm was read on a microtiter plate reader and antibodies with absorbance of the antigen-coated well at least threefold greater than PBS-coated control were specific binding molecules and subjected to sequence analysis.

### Example 2—Recombinant Production and Purification

Codon optimized DNA inserts were cloned into modified pcDNA3.4 (Genewiz) for small scale expression in HEK293 cells in 24 well plates. The binding proteins were purified in substantial accordance with the following procedure. Using a Hamilton Star automated system, 96×4 mL of supernatants in 4×24-well blocks were re-arrayed into 4×96-well, 1 mL blocks. PhyNexus™ micropipette tips (Biotage, San Jose CA) holding 80 μL of Ni-Excel IMAC resin (Cytiva) are equilibrated wash buffer: PBS pH 7.4, 30 mM imidazole. PhyNexus™ tips were dipped and cycled through 14 cycles of 1 mL pipetting across all 4×96-well blocks. PhyNexus™ tips were washed in 2×1 mL blocks holding wash buffer. PhyNexus™ tips were eluted in 3×0.36 mL blocks holding elution buffer: PBS pH 7.4, 400 mM imidazole. PhyNexus™ tips were regenerated in 3×1 mL blocks of 0.5 M sodium hydroxide.

The purified protein eluates were quantified using a Biacore® T200 as in substantial accordance with the following procedure. 10 μL of the first 96×0.36 mL eluates were transferred to a Biacore® 96-well microplate and diluted to 60 μL in HBS-EP+ buffer (10 mM Hepes pH 7.4, 150 mM NaCl, 1 mM EDTA, 0.05% Tween™ 20). Each of the 96 samples was injected on a CM5 series S chip previously functionalized with anti-histidine capture antibody (Cytiva): injection is performed for 18 seconds at 5 μL/min. Capture levels were recorded 60 seconds after buffer wash. A standard curve of known V_{H}H concentrations (270, 90, 30, 10, 3.3, 1.1 μg/mL) was acquired in each of the 4 Biacore® chip flow cells to eliminate cell-to-cell surface variability. The 96 captures were interpolated against the standard curve using a non-linear model including specific and unspecific, one-site binding. Concentrations in the first elution block varied from 12 to 452 μg/mL corresponding to a 4-149 μg. SDS-PAGE analysis of 5 randomly picked samples was performed to ensure molecular weight of eluates corresponded to expected values (˜30 kDa).

The concentration of the proteins was normalized using the Hamilton Star automated system in substantial accordance with the following procedure. Concentration values are imported in an Excel spreadsheet where pipetting volumes were calculated to perform dilution to 50 μg/mL in 0.22 mL. The spreadsheet was imported in a Hamilton Star method dedicated to performing dilution pipetting using the first elution block and elution buffer as diluent. The final, normalized plate was sterile filtered using 0.22 μm filter plates (Corning).

### Example 3

All experiments were conducted in 10 mM Hepes, 150 mM NaCl, 0.05% (v/v) Polysorbate 20 (PS20) and 3 mM EDTA (HBS-EP+ buffer) on a Biacore® T200 instrument equipped with a Protein A chip (Cytiva). Mono-Fe VHH ligands were flowed at 5 μl/min for variable time ranging from 18 to 300 seconds, reaching the capture loads listed in the tables below.

Following ligand capture, injections of a 2-fold dilution series of his-tagged cytokine receptors typically comprising at least five concentrations between 1 μM and 1 nM were performed in either high performance or single cycle kinetics mode. Surface regeneration was achieved by flowing 10 mM glycine-HCl, pH 1.5 (60 seconds, 50 μL/min). Buffer-subtracted sensograms were processed with Biacore® T200 Evaluation Software and globally fit with a 1:1 Langmuir binding model (bulk shift set to zero) to extract kinetics and affinity constants (ka, kd, KD). RMAX<100 RU indicates surface density compatible with kinetics analysis. Calculated Rmax were generated using the equation Rmax=Load (RU)×valency of ligand×(Molecular weight of analyte/Molecular weight of ligand. Surface activity was defined as the ratio experimental/calculated Rmax. See tables below for sample information and experimental results.

Anti-hGP130 Mono-Fc VHHs (Ligand) Binding to hGP130-his (Sino Biological, Catalog #10974)

| | | | | | | Calc. | |
|---|---|---|---|---|---|---|---|
| | k_{ON} | k_{OFF} | Affinity | Rmax | Load | Rmax | Surface |
| Ligand | (1/Ms) | (1/s) | (nM) | (RU) | (RU) | (RU) | Activity |
| 14 - DR591 (SEQ ID NO. | 5.9E+04 | 1.9E−03 | 33 | 52 | 84.6 | 252 | 21% |
| 232) | | | | | | | |
| 15 - DR592 (SEQ ID NO. | 1.6E+05 | 5.9E−03 | 38 | 142.3 | 147 | 437 | 33% |
| 233) | | | | | | | |
| 16 - DR593 (SEQ ID NO. | 2.5E+05 | 2.1E−02 | 82 | 64.2 | 110 | 326 | 20% |
| 234) | | | | | | | |
| 17 - DR594 (SEQ ID NO. | 1.6E+05 | 9.5E−03 | 58 | 96.9 | 153 | 455 | 21% |
| 235) | | | | | | | |
| 18 - DR595 (SEQ ID NO. | 1.8E+05 | 7.3E−03 | 41 | 127 | 128 | 379 | 33% |
| 236) | | | | | | | |
| 19 - DR596 (SEQ ID NO. | 1.9E+05 | 8.2E−03 | 44 | 68.8 | 83.6 | 249 | 28% |
| 237) | | | | | | | |

Anti-hIL27Ra Mono-Fc VHHs (Ligand) Binding to hIL27Ra-his (Origene, Catalog #TP307012)

| | | | | | | Calc. | |
|---|---|---|---|---|---|---|---|
| | k_{ON} | k_{OFF} | Affinity | Rmax | Load | Rmax | Surface |
| Ligand | (1/Ms) | (1/s) | (nM) | (RU) | (RU) | (RU) | Activity |
| 20 - DR597 (SEQ ID NO. | 3.0E+04 | 5.7E−04 | 19.0 | 142* | 1319 | 3564 | 4% |
| 245) | | | | | | | |
| 24 - DR601 (SEQ ID NO. | 9.1E+04 | 4.1E−04 | 4.5 | 102 | 305 | 824 | 12% |
| 249) | | | | | | | |
| 23 - DR600 (SEQ ID NO. | 5.9E+04 | 1.0E−03 | 17 | 79 | 232 | 627 | 13% |
| 248) | | | | | | | |
| 25 - DR602 (SEQ ID NO. | 1.5E+05 | 7.6E−04 | 5.2 | 115* | 210 | 568 | 20% |
| 250) | | | | | | | |
| 26 - DR603 (SEQ ID NO. | 1.3E+05 | 7.7E−04 | 6.1 | 149* | 305 | 823 | 18% |
| 251) | | | | | | | |

*Both association and dissociation kinetics constants might be suppressed at Rmax >100.
If existing, this effect is likely cancelled in the kinetics ratio, i.e. affinity constant.

### Example 4

Binding for all VHH was confirmed by ELISA. One representative VHH from each clonotype was selected for further analysis by surface plasmon resonance using Biacore® T200. See below.

| | | | kON | kOFF | Affinity | kON | kOFF | Affinity |
|---|---|---|---|---|---|---|---|---|
| | hIL27Ra | hgp130 | (1/Ms) | (1/s) | (nM) | (1/Ms) | (1/s) | (nM) |
| Dimer | arm | arm | hIL27Ra | hIL27Ra | hIL27Ra | hgp130 | hgp130 | hgp130 |
| hIL27Ra_VH | VHH1 | DR591 | 3.00E+04 | 5.70E−04 | 19 | 5.90E+04 | 1.90E−03 | 33 |
| H1-DR591 | | | | | | | | |
| hIL27Ra_VH | VHH4 | DR591 | — | — | — | 5.90E+04 | 1.90E−03 | 33 |
| H4-DR591 | | | | | | | | |
| hIL27Ra_VH | VHH6 | DR591 | — | — | — | 5.90E+04 | 1.90E−03 | 33 |
| H6-DR591 | | | | | | | | |
| hIL27Ra_VH | VHH9 | DR591 | 9.10E+04 | 4.10E−04 | 4.5 | 5.90E+04 | 1.90E−03 | 33 |
| H9-DR591 | | | | | | | | |
| hIL27Ra_VH | VHH15 | DR591 | 5.90E+04 | 1.00E−03 | 17 | 5.90E+04 | 1.90E−03 | 33 |
| H15-DR591 | | | | | | | | |
| hIL27Ra_VH | VHH19 | DR591 | 1.50E+05 | 7.60E−04 | 5.2 | 5.90E+04 | 1.90E−03 | 33 |
| H19-DR591 | | | | | | | | |
| hIL27Ra_VH | VHH21 | DR591 | 1.30E+05 | 7.70E−04 | 6.1 | 5.90E+04 | 1.90E−03 | 33 |
| H21-DR591 | | | | | | | | |
| hIL27Ra_VH | VHH1 | DR592 | 3.00E+04 | 5.70E−04 | 19 | 1.60E+05 | 5.90E−03 | 38 |
| H1-DR592 | | | | | | | | |
| hIL27Ra_VH | VHH4 | DR592 | — | — | — | 1.60E+05 | 5.90E−03 | 38 |
| H4-DR592 | | | | | | | | |
| hIL27Ra_VH | VHH6 | DR592 | — | — | — | 1.60E+05 | 5.90E−03 | 38 |
| H6-DR592 | | | | | | | | |
| hIL27Ra_VH | VHH9 | DR592 | 9.10E+04 | 4.10E−04 | 4.5 | 1.60E+05 | 5.90E−03 | 38 |
| H9-DR592 | | | | | | | | |
| hIL27Ra_VH | VHH15 | DR592 | 5.90E+04 | 1.00E−03 | 17 | 1.60E+05 | 5.90E−03 | 38 |
| H15-DR592 | | | | | | | | |
| hIL27Ra_VH | VHH19 | DR592 | 1.50E+05 | 7.60E−04 | 5.2 | 1.60E+05 | 5.90E−03 | 38 |
| H19-DR592 | | | | | | | | |
| hIL27Ra_VH | VHH21 | DR592 | 1.30E+05 | 7.70E−04 | 6.1 | 1.60E+05 | 5.90E−03 | 38 |
| H21-DR592 | | | | | | | | |
| hIL27Ra_VH | VHH1 | DR593 | 3.00E+04 | 5.70E−04 | 19 | 2.50E+05 | 2.10E−02 | 82 |
| H1-DR593 | | | | | | | | |
| hIL27Ra_VH | VHH4 | DR593 | — | — | — | 2.50E+05 | 2.10E−02 | 82 |
| H4-DR593 | | | | | | | | |
| hIL27Ra_VH | VHH6 | DR593 | — | — | — | 2.50E+05 | 2.10E−02 | 82 |
| H6-DR593 | | | | | | | | |
| hIL27Ra_VH | VHH9 | DR593 | 9.10E+04 | 4.10E−04 | 4.5 | 2.50E+05 | 2.10E−02 | 92 |
| H9-DR593 | | | | | | | | |
| hIL27Ra_VH | VHH15 | DR593 | 5.90E+04 | 1.00E−03 | 17 | 2.50E+05 | 2.10E−02 | 82 |
| H15-DR593 | | | | | | | | |
| hIL27Ra_VH | VHH19 | DR593 | 1.50E+05 | 7.60E−04 | 5.2 | 2.50E+05 | 2.10E−02 | 82 |
| H19-DR593 | | | | | | | | |
| hIL27Ra_VH | VHH21 | DR593 | 1.30E+05 | 7.70E−04 | 6.1 | 2.50E+05 | 2.10E−02 | 82 |
| H21-DR593 | | | | | | | | |
| hIL27Ra_VH | VHH1 | DR594 | 3.00E+04 | 5.70E−04 | 19 | 1.60E+05 | 9.50E−03 | 58 |
| H1-DR594 | | | | | | | | |
| hIL27Ra_VH | VHH4 | DR594 | — | — | — | 1.60E+05 | 9.50E−03 | 58 |
| H4-DR594 | | | | | | | | |
| hIL27Ra_VH | VHH6 | DR594 | — | — | — | 1.60E+05 | 9.50E−03 | 58 |
| H6-DR594 | | | | | | | | |
| hIL27Ra_VH | VHH9 | DR594 | 9.10E+04 | 4.10E−04 | 4.5 | 1.60E+05 | 9.50E−03 | 58 |
| H9-DR594 | | | | | | | | |
| hIL27Ra_VH | VHH15 | DR594 | 5.90E+04 | 1.00E−03 | 17 | 1.60E+05 | 9.50E−03 | 58 |
| H15-DR594 | | | | | | | | |
| hIL27Ra_VH | VHH19 | DR594 | 1.50E+05 | 7.60E−04 | 5.2 | 1.60E+05 | 9.50E−03 | 58 |
| H19-DR594 | | | | | | | | |
| hIL27Ra_VH | VHH21 | DR594 | 1.30E+05 | 7.70E−04 | 6.1 | 1.60E+05 | 9.50E−03 | 58 |
| H21-DR594 | | | | | | | | |
| hIL27Ra_VH | VHH1 | DR595 | 3.00E+04 | 5.70E−04 | 19 | 1.80E+05 | 7.30E−03 | 41 |
| H1-DR595 | | | | | | | | |
| hIL27Ra_VH | VHH4 | DR595 | — | — | — | 1.80E+05 | 7.30E−03 | 41 |
| H4-DR595 | | | | | | | | |
| hIL27Ra_VH | VHH6 | DR595 | — | — | — | 1.80E+05 | 7.30E−03 | 41 |
| H6-DR595 | | | | | | | | |
| hIL27Ra_VH | VHH9 | DR595 | 9.10E+04 | 4.10E−04 | 4.5 | 1.80E+05 | 7.30E−03 | 41 |
| H9-DR595 | | | | | | | | |
| hIL27Ra_VH | VHH15 | DR595 | 5.90E+04 | 1.00E−03 | 17 | 1.80E+05 | 7.30E−03 | 41 |
| H15-DR595 | | | | | | | | |
| hIL27Ra_VH | VHH19 | DR595 | 1.50E+05 | 7.60E−04 | 5.2 | 1.80E+05 | 7.30E−03 | 41 |
| H19-DR595 | | | | | | | | |
| hIL27Ra_VH | VHH21 | DR595 | 1.30E+05 | 7.70E−04 | 6.1 | 1.80E+05 | 7.30E−03 | 41 |
| H21-DR595 | | | | | | | | |
| hIL27Ra_VH | VHH1 | DR596 | 3.00E+04 | 5.70E−04 | 19 | 1.90E+05 | 8.20E−03 | 44 |
| H1-DR596 | | | | | | | | |
| hIL27Ra_VH | VHH4 | DR596 | — | — | — | 1.90E+05 | 8.20E−03 | 44 |
| H4-DR596 | | | | | | | | |
| hIL27Ra_VH | VHH6 | DR596 | — | — | — | 1.90E+05 | 8.20E−03 | 44 |
| H6-DR596 | | | | | | | | |
| hIL27Ra_VH | VHH9 | DR596 | 9.10E+04 | 4.10E−04 | 4.5 | 1.90E+05 | 8.20E−03 | 44 |
| H9-DR596 | | | | | | | | |
| hIL27Ra_VH | VHH15 | DR596 | 5.90E+04 | 1.00E−03 | 17 | 1.90E+05 | 8.20E−03 | 44 |
| H15-DR596 | | | | | | | | |
| hIL27Ra_VH | VHH19 | DR596 | 1.50E+05 | 7.60E−04 | 5.2 | 1.90E+05 | 8.20E−03 | 44 |
| H19-DR596 | | | | | | | | |
| hIL27Ra_VH | VHH21 | DR596 | 1.30E+05 | 7.70E−04 | 6.1 | 1.90E+05 | 8.20E−03 | 44 |
| H21-DR596 | | | | | | | | |

LENGTHY TABLES
The patent application contains a lengthy table section. A copy of the table is available in electronic form from the USPTO web site (https://seqdata.uspto.gov/?pageRequest=docDetail&DocID=US20250179198A1). An electronic copy of the table will also be available from the USPTO upon request and payment of the fee set forth in 37 CFR 1.19(b)(3).

1. An IL27 receptor (IL27R) binding protein that specifically binds to IL27Rα subunit (IL27Rα) and glycoprotein 130 subunit (gp130),
wherein the binding protein causes the multimerization of IL27Rα and gp130 when bound to IL27Rα and gp130, and
wherein the binding protein comprises a single-domain antibody (sdAb) that specifically binds to IL27Rα (an anti-IL27Rα sdAb) and a sdAb that specifically binds to gp130 (an anti-gp130 sdAb).

2. The IL27R binding protein of claim 1, wherein the anti-IL27Rα sdAb is a V_{H}H antibody (an anti-IL27Rα V_{H}H antibody) and/or the anti-gp130 sdAb is a V_{H}H antibody (an anti-gp130 V_{H}H antibody).

3-5. (canceled)

6. The IL27R binding protein of claim 2, wherein the anti anti-gp130 V_{H}H antibody comprises a CDR1 having 0, 1, 2, or 3 amino acid changes relative to the sequence of any one of SEQ ID NOS: 193-198; a CDR2 having 0, 1, 2, or 3 amino acid changes relative to the sequence of any one of SEQ ID NOS: 199-204; and a CDR3 having 0, 1, 2, or 3 amino acid changes relative to the sequence of any one of SEQ ID NOS: 205-210; and/or
wherein the anti-IL27Rα V_{H}H antibody comprises a CDR1 having 0, 1, 2, or 3 amino acid changes relative to the sequence of any one of SEQ ID NOS: 211-217; a CDR2 having 0, 1, 2, or 3 amino acid changes relative to the sequence of any one of SEQ ID NOS: 218-224; and a CDR3 having 0, 1, 2, or 3 amino acid changes relative to the sequence of any one of SEQ ID NOS: 225-231.

7. (canceled)

8. The IL27R binding protein of claim 2, wherein the IL27R binding protein comprises:
a first V_{H}H antibody comprising a CDR1 having at least 90% sequence identity, or having 0, 1, 2, or 3 amino acid changes, optionally conservative amino acid changes, relative to the sequence of a CDR1 from a row of Table 1A; a CDR2 having at least 90% sequence identity, or having 0, 1, 2, or 3 amino acid changes, optionally conservative amino acid changes, relative to the sequence of a CDR2 from the same row of Table 1A; and a CDR3 having at least 90% sequence identity, or having 0, 1, 2, or 3 amino acid changes, optionally conservative amino acid changes, relative to the sequence of a CDR3 from the same row of Table 1A; and
a second V_{H}H antibody comprising a CDR1 having at least 90% sequence identity, or having 0, 1, 2, or 3 amino acid changes, optionally conservative amino acid changes, relative to the sequence of a CDR4 from the same row of Table 1A; a CDR2 having at least 90% sequence identity, or having 0, 1, 2, or 3 amino acid changes, optionally conservative amino acid changes, relative to the sequence of a CDR5 from the same row of Table 1A; and a CDR3 having at least 90% sequence identity, or having 0, 1, 2, or 3 amino acid changes, optionally conservative amino acid changes, relative to the sequence of a CDR6 from the same row of Table 1A.

9. The IL27R binding protein of claim 8, wherein the IL27R binding protein comprises a sequence at least 90% identical to any one of the dual V_{H}H dimer sequences shown in Table 1A.

10. (canceled)

11. (canceled)

12. The IL27R binding protein of claim 2, wherein the anti-gp130 V_{H}H antibody comprises a sequence having at least 90% sequence identity to a sequence of any one of SEQ ID NOS: 232-237; and/or wherein the anti-IL27Rα V_{H}H antibody comprises a sequence having at least 90% sequence identity to a sequence of any one of SEQ ID NOS: 238-244.

13. (canceled)

14. (canceled)

15. The IL27R binding protein of claim 1, wherein the binding protein comprises a sequence having at least 90% identity to a sequence of any one of SEQ ID NOS: 1-42.

16. (canceled)

17. The IL27R binding protein of claim 2, wherein the anti-IL27Rα V_{H}H antibody comprises a sequence having at least 90% sequence identity to a sequence of any one of SEQ ID NOS: 245-251; and/or wherein the anti-gp130 V_{H}H antibody comprises a sequence having at least 90% sequence identity to a sequence of any one of SEQ ID NOS: 252-257.

18. (canceled)

19. (canceled)

20. The IL27R binding protein of claim 1, wherein the binding protein comprises a sequence having at least 90% identity to a sequence of any one of SEQ ID NOS: 43-84 and 406, 430, 454, 478, 502, 526, 409, 433, 457, 481, 505, 529, 411, 435, 459, 483, 507, 531, 414, 438, 462, 486, 510, 534, 420, 444, 468, 492, 516, 540, 424, 448, 472, 496, 520, 544, 426, 450, 474, 498, 522, and 546.

21. An isolated nucleic acid encoding the IL27R binding protein of claim 1.

22. The isolated nucleic acid of claim 21, wherein the isolated nucleic acid comprises a sequence having at least 90% sequence identity to a sequence of any one of SEQ ID NOS: 109-192 or a sequence in Table 1B.

23. An expression vector comprising the nucleic acid of claim 21.

24. An isolated host cell comprising the vector of claim 23.

25. A pharmaceutical composition comprising the IL27R binding protein of claim 1 and a pharmaceutically acceptable carrier.

26. A method of treating an autoimmune or inflammatory disease, disorder, or condition, a neoplastic disease or a viral infection in a subject in need thereof, comprising administering to the subject a therapeutically effective amount of an IL27R binding protein of claim 1.

27. The method of claim 26, further comprising administering one or more supplementary agents selected from the group consisting of a corticosteroid, a Janus kinase inhibitor, a calcineurin inhibitor, a mTor inhibitor, an IMDH inhibitor, a biologic, a vaccine, and a therapeutic antibody.

28. (canceled)

29. The method of claim 26, wherein the disease, disorder, or condition is selected from viral infections, Helicobacter pylori infection, HTLV, organ rejection, graft versus host disease, autoimmune thyroid disease, multiple sclerosis, allergy, asthma, neurodegenerative diseases including Alzheimer's disease, systemic lupus erythramatosis (SLE), autoinflammatory diseases, inflammatory bowel disease (IBD), Crohn's disease, diabetes, cartilage inflammation, arthritis, rheumatoid arthritis, juvenile arthritis, juvenile rheumatoid arthritis, juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reiter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoidarthritis, polyarticular rheumatoidarthritis, systemic onset rheumatoidarthritis, ankylosing spondylitis, enteropathic arthritis, reactive arthritis, Reiter's syndrome, SEA Syndrome, psoriasis, psoriatic arthritis, dermatitis (eczema), exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid, urticaria, prokeratosis, rheumatoid arthritis; seborrheic dermatitis, solar dermatitis; seborrheic keratosis, senile keratosis, actinic keratosis, photo-induced keratosis, keratosis follicularis; acne vulgaris; keloids; nevi; warts including verruca, condyloma or condyloma acuminatum, and human papilloma viral (HPV) infections.

30. The IL27R binding protein of claim 20, wherein the IL27R binding protein comprises a sequence of any one of SEQ ID NOS: 406, 430, 454, 478, 502, 526, 409, 433, 457, 481, 505, 529, 411, 435, 459, 483, 507, 531, 414, 438, 462, 486, 510, 534, 420, 444, 468, 492, 516, 540, 424, 448, 472, 496, 520, 544, 426, 450, 474, 498, 522, and 546.

31. The IL27R binding protein of claim 30, wherein the IL27R binding protein comprises a sequence of SEQ ID NO: 486 or 496.

32. The IL27R binding protein of claim 30, wherein the IL27R binding protein comprises a sequence of SEQ ID NO: 486.

33. The method of claim 26, wherein the disease, disorder, or condition is inflammatory bowel disease (IBD), Crohn's disease, or ulcerative colitis.

34. One or more expression vectors comprising an anti-IL27Rα V_{H}H antibody and an anti-gp130 V_{H}H antibody of claim 2.
